SEQUENCE LISTING (165 sequences)

SEQ ID NO: 1 - protein, 377 aa, Bos taurus
MCDEDETTALVCDNGSGLVKAGFAGDDAPRAVFPSIVGRPRHQGVMVGMGQKDSYVGDEAQSKKGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 2 - protein, 377 aa, Sus scrofa
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 3 - protein, 377 aa, Gallus gallus; identical to SEQ ID NO: 2

SEQ ID NO: 4 - protein, 377 aa, Bos taurus; identical to SEQ ID NO: 2

SEQ ID NO: 5 - protein, 168 aa, Gallus gallus
MAPKKAKRRAAEGSSNVFSMFDQTQIQEFKEAFTVIDQNRDGIIDKDDLRETFAAMGRLNVKNEELDAMIKEASGPINFTVFLTMFGEKLKGADPEDVIMGAFKVLDPDGKGSIKKSFLEELLTTQCDRFTPEEIKNMWAAFPPDVAGNVDYKNICYVITHGEDKEGE

SEQ ID NO: 6 - protein, 1940 aa, Gallus gallus
MSSDAEMAIFGEAAPYLRKSEKERIEAQNKPFDAKTSVFVVHAKESYVKSTIQSKESGKVTVKTESGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGDKKKEEQPAGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDYHYVSQGEITVPSINDQEELMATDSAIDILGFTPDEKTAIYKLTGAVMHYGNLKFKQKQREEQAEPDGTEVADKAAYLMGLNSADLLKALCYPRVKVGNEFVTKGQTVQQVYNSVGALAKAVFEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNISGWLDKNKDPLYETVVGLYQKSSLKTLALLFASAGGEAESGGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQARCRGFLMRVEYRRMVERRESIFCIQYNIRSFMNVKHWPWMKLFFKIKPLLKSAESEKEMANMKGEFEKTKEELAKSGAKRKDLEGKMVSLLQEKNDLQLQVQAEADALADAEERCDQLIKTKIQLEAKIKEVTERAEDEEEINAELTAKKRKLEDECSELKKDIDDLELTLAKVEKEKHATENKVKNLTEEMAALDENIAKLTKEKKAPQEAHQQTLDDLQAEEDKVNTLTKAKTKLEQQVDDLEGSLEQEKKLRMDLERAKRKLEGDLKLAHDSIMDLENDKQQLDEKLKKKDFEISQIQSKIEDEQALGMQLQKKIKELQARTEELEEEIEAERTSRAKAEKHRADLSRELEEISERLEEAGGATAAQIDMNKKREAEFQKMRRDLEEATLQHEATAAALRKKHADSTAELGEQIDNLQRVKQKLEKEKSELKMEIDDLASNMESVSKAKASLEKTCRALEDQMSEIKTKEEEHQRMINDVNAQRARLQTESGEYSRQVEEKDALISQLSRGKQAFTQQIEELKRHLEEEIKAKNALAHGLQSARHDCDLLREQYEEEQEAKGELQRALSKANSEVAQWRTKYETDAIQRTEELEEAKKKLAQRLQDAEEHVEAVNSKCASLEKTKQRLQNEVEDLMIDVERANSACAALDKKQKNFDKILSEWKQKYEETQAELEASQKESRSLSTELFKMKNAYEESLDHLETLKRENKNLQQEISDLTEQIAEGGKAIHELEKVKKQIEQEKSELQASLEEAEASLEHEEGKILRLQLELNQVKSEIDRKIAEKDEEIDQLKRNHLRIVESMQRTLDAEVRSRNEALRLKKKMEGDLNEMEIQLNHANRMAAEAQKNLRNTQGVLKDTQIHLDDALRSQEDLKEQVAMVERRANLLQAETEELRAALEQTERSRKVAEQELLDASERVQLLHTQNTSLINTKKKLESDISQIQSEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNVLRLQDLVDKLQMKVKSYKRQAEEAEELSNVNLSKFRKIQHELEEAEERADIAESQVNKLRVKSREFHKKIEEEEI

SEQ ID NO: 7 - protein, 1937 aa, Salmo salar
MSTDAEMQVYGKAAIYLRKSEKERMEAQAMPFDSKNSCYVTDKVELYLKGLVTARADGKCTVTVTKPDGTKEEGKEFKDADIYEMNPPKYDKIEDMAMMTYLNEASVLYNLKERYAAWMIYTYSGLFCATVNPYKWLPVYDEEVVNAYRGKKRVEAPPHIFSVSDNAFQFMMIDKENQSVLITGESGAGKTVNTKRVIQYFATIAVSGGKKEADPNKMQGSLEDQIIAANPLLESYGNAKTVRNDNSSRFGKFIRIHFQAGKLAKADIETYLLEKSRVSFQLPDERGYHIFFQMMTGHKPELVELALLTTNPYDFPMCSQGQIAVASINDNEELDATDEAITILGFTNEEKLGIYKLTGAVVHHGNLKFKQKQREEQAEPDGTEVADKIAYLLGLNSAEMLKALCYPRVKVGNEYVTKGQTVAQVNNSVSALAKSIYERMFLWMVIRINEMLDTKNPRQFYIGVLDIAGFEIFDYNSMEQLCINFTNEKLQQFFNHTMFVLEQEEYKKEGIVWAFIDFGMDLAACIELIEKPLGIFSILEEECMFPKSSDTTFKDKLYAQHLGKTKAFEKPKPAKGKAEAHFSLVHYAGTVDYNITGWLEKNKDPLNDSVCQLYGKSGVKILAALYPPPPPEDKAKKGGKKKGGSMQTVSSQFRENLHKLMTNLRSTHPHFVRCLIPNESKTPGLMENFLVIHQLRCNGVLEGIRICRKGFPSRIIYADFKQRYKVLNASVIPEGQFMDNKKASEKLLGSIDVNHEDYKFGHTKVFFKAGLLGVLEEMRDEKLATLVGMVQALSRGFLMRREFSKMMERRESIYAIQYNIRSFMNVKTWPWMKLYFKIKPLLQSAETEKELANMKENYEKMKTDLAKALSTKKQMEEKLVSLTQEKNDLALQVASEGESLNDAEERCEGLIKSKIQQEAKLKETTERLEDEEEINAELTAKKRKLEDECSELKKDIDDLELTLAKVEKEKHATENKVKNLTEEMASMDESVAKLTKEKKALQEAHQQTLDDLQAEEDKVNTLTKAKTKLEQQVDDLEGSLEQEKKLRMDLERAKRKLEGDLKLAQESIMDLENDKQQADEKIKKKEFETTQLLSKIEDEQSLGAQLQKKIKELQARIEELEEEIEAERAARAKVEKQRADLSRELEEISERLEEAGGATAAQIEMNKKREAEFQKLRRDLEESTLQHEATAAALRKKQADSVAELGEQIDNLQRVKQKLEKEKSEYKMEIDDLSSNMEAVAKAKGNLEKMCRTLEDQLSELKTKNDENVRQVNDISGQRARLLTENGEFGRQLEEKEALVSQLTRGKQAFTQQVEELKRLIEEEVKAKNALAHGVQSARHDCDLLREQFEEEQEAKAELQRGMSKANSEVAQWRTKYETDAIQRTEELEEAKKKLAQRLQEAEETIEATNSKCASLEKTKQRLQGEVEDLMIDVERANALAANLDKKQRNFDKVLAEWKQKYEEGQAELEGAQKEARSMSTELFKMKNSYEEALDHLETLKRENKNLQQEISDLTEQIGETGKSIHELEKAKKTVETEKSEIQTALEEAEGTLEHEESKILRVQLELNQIKGEVDRKIAEKDEEMEQIKRNSQRVVDSMQSTLDSEVRSRNDALRVKKKMEGDLNEMEIQLSHSNRQAAEAQKQLRNVQGQLKDAQLHLDDAVRAAEDMKEQAAMVERRNGLMVAEIEELRVALEQTERGRKVAETELVDASERVGLLHSQNTSLLNTKKKLETDLVQVQGEVDDIVQEARNAEEKAKKAITDAAMMAEELKKEQDTSSHLERMKKNLEVTVKDLQHRLDEAENLAMKGGKKQLQKLESRVRELETEVEAEQRRGVDAVKGVRKYERRVKELTYQTEEDKKNVNRLQDLVDKLQMKVKAYKRQAEEAEEAANQHMSKFRKVQHELEEAEERADIAETQVNKLRAKTRDSGKGKEAAE

SEQ ID NO: 8 - protein, 832 aa, Salmo salar
MSTDAEMQIYGKAAIYLRKSEKERMEAQAAPFDSKNSCYVADKVELYLKGLITARADGKCTVTVTKPDGTKEEGKEFKDADIYEMNPPKYDKIEDMAMMTYLNEASVLYNLKERYAAWMIYTYSGLFCATVNPYKWLPVYDAEVVNAYRGKKRMEAPPHIFSVSDNAFQFMLIDKENQSVLITGESGAGKTVNTKRVIQYFATIAVSGGEKKKEVDPSKMQGSLEDQIIAANPLLEAYGNAKTVRNDNSSRFGKFIRIHFQGGKLAKADIETYLLEKSRVSFQLPDERGYHIFFQMMTGHKPEIVEMALITTNPYDFPMCSQGQIAVASIDDKEELDATDDAITILGFTNDEKIGIYKLTGAVVHHGNLKFKQKQREEQAEPDGTEVADKIGYLLGLNSAEMLKALCYPRVKVGNEYVTKGQTVPQVNNSVMALAKSIYERMFLWMVIRINEMLDTKNPRQFYIGVLDIAGFEIFDYNSMEQLCINFTNEKLQQFFNHTMFVLEQEEYKKEGIVWAFIDFGMDLAACIELIEKPLGIFSILEEECMFPKSSDTTFKDKLYSQHLGKTQAFEKPKPAKGKAEAHFSLVHYAGTVDYNITGWLEKNKDPLNDSVCQLYGKSGVKILAALYPAAPPEDTTKKGGKKKGGSMQTVSSQFRENLHKLMTNLRSTHPHFVRCLIPNESKTPGLMENFLVIHQLRCNGVLEGIRICRKGFPSRIIYADFKQRYKVLNASVIPEGQFMDNKKASEKLLGSIDVNHEDYKFGHTKVSQILYFKIKPLLQSAETEKELANMKENYEKMTADLAKALSTKKQMEEKLVALMQEKNDLALQVAS

SEQ ID NO: 9 - protein, 192 aa, Bos taurus
MAPKKDVKKPAAAAAPAPAPAPAPAPAPAPPKEEKIDLSAIKIEFSKQQQDEFKEAFLLFDRTGECKITLSQVGDVLRALGTNPTNAEVKKVLGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSN

SEQ ID NO: 10 - protein, 192 aa, Gallus gallus
MAPKKDVKKPAAAAAPAPAPAPAPAPAPAKPKEPAIDLKSIKIEFSKEQQDDFKEAFLLFDRTGDAKITLSQVGDIVRALGQNPTNAEINKILGNPSKEEMNAKKITFEEFLPMLQAAANNKDQGTFEDFVEGLRVFDKEGNGTVMGAELRHVLATLGEKMTEEEVEELMKGQEDSNGCINYEAFVKHIMSV

SEQ ID NO: 11 - protein, 150 aa, Gallus gallus
MSFSPDEINDFKEAFLLFDRTGDAKITLSQVGDIVRALGQNPTNAEINKILGNPSKEEMNAKKITFEEFLPMLQAAANNKDQGTFEDFVEGLRVFDKEGNGTVMGAELRHVLATLGEKMTEEEVEELMKGQEDSNGCINYEAFVKHIMSV

SEQ ID NO: 12 - protein, 161 aa, Salmo salar
MADAAPAEASGASAFTADQIEDFKEAFGLFDRVGDSMIGYNQVADVMRALGQNPQNKEVAAILGKPSADDMANKRLAFADFMPMMEKVDKIVKGTLDDYVEGLRVFDKEGNGTVSGAELRIVLGTLGEKMSEAEIDSLLIGQEDENGSINYEAFVKHVLSV

SEQ ID NO: 13 - protein, 1939 aa, Gallus gallus
MASPDAEMAAFGEAAPYLRKSEKERIEAQNKPFDAKSSVFVVHPKESFVKGTIQSKEGGKVTVKTEGGETLTVKEDQVFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQSGKMQGTLEDQIISANPLLEAFGNAKTVRNDNSSRFGKFIRIHFGATGKLASADIETYLLEKSRVTFQLPAERSYHIFYQIMSNKKPELIDMLLITTNPYDYHYVSQGEITVPSIDDQEELMATDSAIDILGFSADEKTAIYKLTGAVMHYGNLKFKQKQREEQAEPDGTEVADKAAYLMGLNSAELLKALCYPRVKVGNEFVTKGQTVSQVHNSVGALAKAVYEKMFLWMVIRINQQLDTKQPRQYFIGVLDIAGFEIFDFNSFEQLCINFTNEKLQQFFNHHMFVLEQEEYKKEGIEWEFIDFGMDLAACIELIEKPMGIFSILEEECMFPKATDTSFKNKLYDQHLGKSNNFQKPKPAKGKAEAHFSLVHYAGTVDYNISGWLEKNKDPLNETVIGLYQKSSVKTLALLFATYGGEAEGGGGKKGGKKKGSSFQTVSALFRENLNKLMANLRSTHPHFVRCIIPNETKTPGAMEHELVLHQLRCNGVLEGIRICRKGFPSRVLYADFKQRYRVLNASAIPEGQFMDSKKASEKLLGSIDVDHTQYRFGHTKVFFKAGLLGLLEEMRDDKLAEIITRTQARCRGFLMRVEYRRMVERRESIFCIQYNVRSFMNVKHWPWMKLFFKIKPLLKSAESEKEMANMKEEFEKTKEELAKSEAKRKELEEKMVVLLQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAVLDETIAKLTKEKKALQEAHQQTLDDLQVEEDKVNTLTKAKTKLEQQVDDLEGSLEQEKKLRMDLERAKRKLEGDLKLAHDSIMDLENDKQQLDEKLKKKDFEISQIQSKIEDEQALGMQLQKKIKELQARIEELEEEIEAERTSRAKAEKHRADLSRELEEISERLEEAGGATAAQIEMNKKREAEFQKMRRDLEEATLQHEATAAALRKKHADSTAELGEQIDNLQRVKQKLEKEKSELKMEIDDLASNMESVSKAKANLEKMCRTLEDQLSEIKTKEEQNQRMINDLNTQRARLQTETGEYSRQAEEKDALISQLSRGKQGFTQQIEELKRHLEEEIKAKNALAHALQSARHDCELLREQYEEEQEAKGELQRALSKANSEVAQWRTKYETDAIQRTEELEEAKKKLAQRLQDAEEHVEAVNAKCASLEKTKQRLQNEVEDLMVDVERSNAACAALDKKQKNFDKILAEWKQKYEETQTELEASQKESRSLSTELFKMKNAYEESLDHLETLKRENKNLQQEIADLTEQIAEGGKAVHELEKVKKHVEQEKSELQASLEEAEASLEHEEGKILRLQLELNQIKSEIDRKIAEKDEEIDQLKRNHLRIVESMQSTLDAEIRSRNEALRLKKKMEGDLNEMEIQLSHANRMAAEAQKNLRNTQGTLKDTQIHLDDALRTQEDLKEQVAMVERRANLLQAEVEELRGALEQTERSRKVAEQELLDATERVQLLHTQNTSLINTKKKLETDIVQIQSEMEDTIQEARNAEEKAKKAITDAAMMAEELKKEQDTSAHLERMKKNMDQTVKDLHVRLDEAEQLALKGGKKQLQKLEARVRELEGEVDSEQKRSAEAVKGVRKYERRVKELTYQCEEDRKNILRLQDLVDKLQMKVKSYKRQAEEAEELSNVNLSKFRKIQHELEEAEERADIAESQVNKLRVKSREIHGKKIEEEE

SEQ ID NO: 14 - protein, 947 aa, Gallus gallus
MRDLLVVPLGHVLTLAALSLAETLQKAPFISTPLETVDALVEDVPKFVCVVESYPEPEITWTRNSIPIRLFDTRYSIQRNGQLLTILSVEDSDDGVYCCTADNGVGAAAQSCGALQVKMRPKITRPPVNVEIIEGLKAVLPCTTMGNPKPSVSWIKGETVVKENARIAVLDSGNLRIHNVQREDAGQYRCVAKNSLGSAYSKPATVVVEVFARILKAPESQNITFGSMVTLRCTAAGAPVPTVTWLENGKAVSAGSIAESVKDRVVDSRLQVYVTRPGLFTCLATNKHSKTFGAAKAAATISVSEWSKLYKGDAGYCSTYRGEVCSAILSRNALVFFNSSYADPEETQELLVHTAWTELKTVSSFCQPAAESLLCNYIFQECKPSGVGPAPKPICRENCLAVKDLYCFKEWLSMEENSQRGIYKPGLMLLALPECNRLPSLHQDPSACTHIPFFDFKKENITRTCYSGNGQFYQGWANVTASGIPCQKWSDQAPHLHRRTPQVFPELSDAENYCRNPGGENERPWCYTKDPSVTWEYCSVSPCGDASLSLGTRKPNGETQNLPPPPSYSPTYSMNVIILIISSFALIVILGIITLVCCRRRKQWKNKKRESETPTLTTLPSELLLDRLHPNPMYQRMPLLLNPKLLSLEYPRNNIEYVRDIGEGAFGRVFQARAPGLLPYEPFTMVAVKMLKEEASADMQADFQREAALMAEFDNPNIVKLLGVCAVGKPMCLLFEYMAYGDLNEYLRDRSPRNLCSLVQGGLEARACLLNPLALCCTSQLCIAKQVAAGMAYLSERKFVHRDLATRNCLVGENMVVKIADFGLSRNMYSADYYKANENDAIPIRWMPPESIFYNRYTTESDVWAYGVVLWEIFSYGMQPYYGMAHEEVIYYVRDGNILSCPDNCPLELYNLMRLCWSKLPADRPSFASIHRILERMYERAVASPQV

SEQ ID NO: 15 - protein, 163 aa, Gallus gallus
MASMTDQQAEARAFLSEEMIAEFKAAFDMFDADGGGDISTKELGTVMRMLGQNPTKEELDAIIEEVDEDGSGTIDFEEFLVMMVRQMKEDAKGKSEEELANCFRIFDKNADGFIDIEELGEILRATGEHVTEEDIEDLMKDSDKNNDGRIDFDEFLKMMEGVQ

SEQ ID NO: 16 - protein, 159 aa, Sus scrofa
TDQQAEARSYLSEEMIAEFKAAFDMFDADGGGDISVKELGTVMRMLGQTPTKEELDAIIEEVDEDGSGTIDFEEFLVMMVRQMKEDAKGKSEEELAECFRIFDRNMDGYIDAEELAEIFRASGEHVTDEEIESIMKDGDKNNDGRIDFDEFLKMMEGVQ

SEQ ID NO: 17 - protein, 183 aa, Gallus gallus
MSDEEKKRRAATARRQHLKSAMLQLAVTEIEKEAAAKEVEKQNYLAEHCPPLSLPGSMQELQELCKKLHAKIDSVDEERYDTEVKLQKTNKELEDLSQKLFDLRGKFKRPPLRRVRMSADAMLRALLGSKHKVNMDLRANLKQVKKEDTEKEKDLRDVGDWRKNIEEKSGMEGRKKMFEAGES

SEQ ID NO: 18 - protein, 271 aa, Bos taurus
MSDEEVEHVEEEYEEEEEAQEEAPPPPAEVPEVHEEVHEVHEPEEVQEEEKPRPRLTAPKIPEGEKVDFDDIQKKRQNKDLMELQALIDSHFEARKKEEEELVALKERIEKRRAERAEQQRIRAEKERERQNRLAEEKARREEEDAKRRAEDDLKKKKALSSMGANYSSYLAKADQKRGKKQTAREMKKKVLAERRKPLNIDHLSEDKLRDKAKELWDTLYQLETDKFEYGEKLKRQKYDITNLRSRIDQAQKHSKKAGTAPKGKVGGRWK

SEQ ID NO: 19 - protein, 262 aa, Sus scrofa
MSDAEEQEYEEEQPEEEEAAEEEEAPEEPEPVAEREEERPKPSRPVVPPLIPPKIPEGERVDFDDIHRKRMEKDLLELQTLIDVHFEQRKKEEEELVALKERIERRRAERAEQQRFRTEKERERQAKLAEEKMRKEEEEAKKRAEDDAKKKKVLSNMGAHFGGYLVKAEQKRGKRQTGREMKQRILSERKKPLNIDHMGEDQLREKAQELSDWIHQLESEKFDLMAKLKQQKYEINVLYNRISHAQKFRKGAGKGRVGGRWK

SEQ ID NO: 20 - protein, 347 aa, Gallus gallus
MTSYRQELEKYRDIDEDKILQELSAEELEQLDTELLEMDPENVLLPAGLRQRDQTQKSPTGPLDREALLQHLEKQALEAKEREDLVPFTGEKKGKPFVPKNPTREIPREEQITLEPELEEALANATEAEMCDIAAILGMYTLMSNKQYYDAICSGTITNTEGINSVVKPDKYKPVPDEPPNPTNVEETLRQIQANDSALEDVNLNNIKDIPISTLKAICEAMKTNTHVKKLSLVATRSNDPVATAVAEMLAENKTLQSLNIESNFITSAGMMSVIKAMYQNSTLSELKVDNQCQRLGNTVEMEMATMLEQCPSVVRFGYHFTQQGPRARAAIAITRNNELRRKQKKT

SEQ ID NO: 21 - protein, 377 aa, Gallus gallus; identical to SEQ ID NO: 2

SEQ ID NO: 22 - protein, 897 aa, Gallus gallus
MNSMNQIETNMQYTYNYEEDEYMTQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHLSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHEQWAYGKEQILLQKDYESASLTEVRAMLRKHEAFESDLAAHQDRVEQIAAIAQELNELDYHDAASVNDRCQKICDQWDSLGTLTQKRREALERTEKLLETIDQLHLEFAKRAAPFNNWMEGAMEDLQDMFIVHSIEEIQSLISAHDQFKATLPEADGERQAILSIQNEVEKVIQSYSMRISASNPYSTVTVEEIRTKWEKVKQLVPQRDQSLQEELARQHANERLRRQFAAQANVIGPWIQTKMEEIARSSIEMTGPLEDQMNQLKQYEQNIINYKHNIDKLEGDHQLIQEALVFDNKHTNYTMEHIRVGWELLLTTIARTINEVETQILTRDAKGITQEQMNDFRASFNHFDRRKNGLMDHDDFRACLISMGYDLGEAEFARIMSLVDPNGQGTVTFQSFIDFMTRETADTDTAEQVIASFRILASDKPYILADELRRELPPEQAQYCIKRMPQYTGPGSVPGALDYTSFSSALYGESDL

SEQ ID NO: 23 - protein, 923 aa, Gallus gallus
MNSMNQIETNMQYTYNYEEDEYMTQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHLSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHEQWAYGKEQILLQKDYESASLTEVRAMLRKHEAFESDLAAHQDRVEQIAAIAQELNELDYHDAASVNDRCQKICDQWDSLGTLTQKRREALERTEKLLETIDQLHLEFAKRAAPFNNWMEGAMEDLQDMFIVHSIEEIQSLISAHDQFKATLPEADGERQAILSIQNEVEKVIQSYSMRISASNPYSTVTVEEIRTKWEKVKQLVPQRDQSLQEELARQHANERLRRQFAAQANVIGPWIQTKMEEIARSSIEMTGPLEDQMNQLKQYEQNIINYKHNIDKLEGDHQLIQEALVFDNKHTNYTMEHIRVGWELLLTTIARTINEVETQILTRDAKGITQEQMNDFRASFNHFDRRKNGLMDHDDFRACLISMGYDLDESDNLHSDEFKACLISLGEVGNDLQGEAEFARIMSLVDPNGQGTVTFQSFIDFMTRETADTDTAEQVIASFRILASDKPYILADELRRELPPEQAQYCIKRMPQYTGPGSVPGALDYTSFSSALYGESDL

SEQ ID NO: 24 - protein, 286 aa, Gallus gallus
MADFEDRVSDEEKVRIAAKFITHAPPGEFNEVFNDVRLLLNNDNLLREGAAHAFAQYNMDQFTPVKIEGYDDQVLITEHGDLGNGRFLDPRNKISFKFDHLRKEASDPQPEDTESALKQWRDACDSALRAYVKDHYPNGFCTVYGKSIDGQQTIIACIESHQFQPKNFWNGRWRSEWKFTITPPTAQVAAVLKIQVHYYEDGNVQLVSHKDIQDSVQVSSDVQTAKEFIKIIENAENEYQTAISENYQTMSDTTFKALRRQLPVTRTKIDWNKILSYKIGKEMQNA

SEQ ID NO: 25 - protein, 286 aa, Gallus gallus
MADLEEQLSDEEKVRIAAKFIIHAPPGEFNEVFNDVRLLLNNDNLLREGAAHAFAQYNLDQFTPVKIDGYDEQVLITEHGDLGNGKFLDPKNKISFKFDHLRKEATDPRPHEVENAIESWRNSVETAMKAYVKEHYPNGVCTVYGKTIDGQQTIIACIESHQFQAKNFWNGRWRSEWKFTISPSTTQVAGILKIQVHYYEDGNVQLVSHKDIQDSLTVSNEAQTAKEFIKIVEAAENEYQTAISENYQTMSDTTFKALRRQLPVTRTKIDWNKILSYKIGKEMQNA

SEQ ID NO: 26 - protein, 286 aa, Gallus gallus; identical to SEQ ID NO: 25

SEQ ID NO: 27 - protein, 290 aa, Gallus gallus
MSVGQGLCESEKVSLICGLMRQSPPGEFRQVVQDLCDLLQDDELVKQQAARAGARHNKNNFTPVLVNGNTVLLTQYNDLGGNRFFYPQDKFSFEFDHLSGVTSKTHLHRVMLDEGELWRGALHKGLNAYVNYHFPVGNCCVFKKSLGKRQMLVACIEAHQYQPSKHWNSLWKSDWTFSLTPVMTRVTGIFLLQLHYFRNANLHVTISKSVSESLHVIDRNQFVTDFVKFVKTEDNKIHNAILENIQALSEHTWRKNLRRRLPITRTFMNWNELLNNQHLKTGVSRKEVPP

SEQ ID NO: 28 - protein, 1940 aa, Gallus gallus
MATDADMAIFGEAAPYLRKSEKERIEAQNKPFDAKSSVFVVHAKESYVKSTIQSKESGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGDKKKEEQPAGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDYQYVSQGEITVPSINDQEELMATDSAIDILGFTPDEKTAIYKLTGAVMHYGNLKFKQKQREEQAEPGGTEVADKAAYLMGLNSADLLKALCYPRVKVGNEYVTKGQTVQQVYNSVGALAKSVFEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNITGWLEKNKDPLNETVVGLYQKSSLKTLALLFASVGGAEAESGAGGKKGGKKKGSSFQTVSALFRENLNKLMSNLRSTHPHFVRCLIPNETKTPGAMEHELVLHQLRCNGVLEGIRICRKGFPIRILYADFKQRYKVLNASAIPEGQFIDSKKASEKLLGSIDVDHTQYKFGHTKVFFKAGLLGLLEEMRDEKLAQLITRTQARCRGFLMRVEFKKMMERRESIFCIQYNVRAFMNVKHWPWMKLFFKIKPLLKSAESEKEMANMKEEFEKTKEELAKSEAKRKELEEKMVSLLQEKNDLQLQVQAEADGLADAEERCDQLIKTKIQLEAKIKELTERAEDEEEMNAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKMTQESTMDLENDKQQLDEKLKKKDFEISQIQSKIEDEQALGMQLQKKIKELQARIEELEEEIEAERTSRAKAEKHRADLSRELEEISERLEEAGGATAAQIDMNKKREAEFQKMRRDLEEATLQHEATAAALRKKHADSTADVGEQIDNLQRVKQKLEKEKSELKMEIDDLASNMESVSKAKANLEKMCRSLEDQLSEIKTKEEEQQRTINDISAQKARLQTESGEYSRQVEEKDALISQLSRGKQAFTQQIEELKRHLEEEIKAKKCPAHALQSARHDCDLLREQYEEEQEAKGELQRALSKANSEVAQWRTKYETDAIQRTEELEEAKKKLAQRLQDAEEHVEAVNSKCASLEKTKQRLQNEVEDLMIDVERSNAACAALDKKQKNFDKILSEWKQKYEETQAELEASQKESRSLSTELFKMKNAYEESLDHLETLKRENKNLQQEISDLTEQIAEGGKAIHELEKVKKQIEQEKSELQTALEEAEASLEHEEGKILRVQLELNQVKSDIDRKIAEKDEEIDQLKRNHLRVVDSMQSTLDAEIRSRNEALRLKKKMEGDLNEIEIQLSHANRQAAEAQKNLRNTQGVLKDTQIHLDDALRSQEDLKEQVAMVERRANLLQAEIEELRAALEQTERSRKVAEQELLDASERVQLLHTQNTSLINTKKKLESDISQIQSEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNVLRLQDLVDKLQMKVKSYKRQAEEAEELSNVNLSKFRKIQHELEEAEERADIAESQVNKLRAKSREIGKKAESEE

SEQ ID NO: 29 - protein, 1939 aa, Sus scrofa
MSSDQEMAIFGEAAPYLRKSEKERIEAQNRPFDAKTSVFVAEPKESFVKGTIQSREGGKVTVKTEAGATLTVKEDQVFPMNPPKFDKIEDMAMMTHLHEPGVLYNLKERYAAWMIYTYSGLFCVTVNPYKWLPVYNPEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQITSNRKPELIEMLLITTNPYDYPFISQGEISVASIDDQEELIATDSAIDILGFTNEEKVSIYKLTGAVMHYGNLKFKQKQREEQAEPDGTEVADKAAYLQSLNSADLLKALCYPRVKVGNEYVTKGQTVEQVTNAVGALAKAVYEKMFLWMVTRINQQLDTKQPRQYFIGVLDIAGFEIFDFNSLEQLCINFTNEKLQQFFNHHMFVLEQEEYKREGIEWTFIDFGMDLAACIELIEKPMGIFSILEEECMFPKATDTSFKNKLYEQHLGKSANFQKPKPAKGKVEAHFSLIHYAGTVDYNITGWLDKNKDPLNDTVVGLYQKSALKTLAFLFSGAQTGEAEAGGTKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQYIDSKKASEKLLASIDIDHTQYKFGHTKVFFKAGLLGLLEEMRDDKLAQLITRTQARCRGFLARVEYQKMVERRESIFCIQYNIRAFMNVKHWPWMKLFFKIKPLLKSAESEKEMATMKEEFQKTKDELAKSEAKRKELEEKMVTLLKEKNDLQLQVQAEAEGLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESIMDIENEKQQLDEKLKKKEFEISNLQSKIEDEQALAIQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRTLEDQLSELKSKEEEQQRLINDLTAQRGRLQTESGEFSRQLDEKEALVSQLSRGKQAYTQQIEELKRQLEEEIKAKNALAHALQSSRHDCDLLREQYEEEQESKAELQRALSKANTEVAQWRTKYETDAIQRTEELEEAKKKLAQRLQAAEEHVEAVNAKCASLEKTKQRLQNEVEDLMLDVERTNAACAALDKKQRNFDKILAEWKQKYEETHAELEASQKEARSLGTELFKMKNAYEESLDQLETLKRENKNLQQEISDLTEQIAEGGKRIHELEKIKKQVEQEKSEIQAALEEAEASLEHEEGKILRIQLELNQVKSEVDRKIAEKDEEIDQLKRNHVRVVESMQSMLDAEIRSRNDAIRLKKKMEGDLNEMEIQLNHANRMAAEALRNYRNTQGILKDTQIHLDDALRGQEDLKEQLAMVERRANLLQAEIEELRATLEQTERSRKVAEQELLDASERVQLLHTQNTSLINTKKKLETDISQMQGEMEDILQEARNAEEKAKKAITDAAMMAEELKKEQDTSAHLERMKKNMEQTVKDLQHRLDEAEQLALKGGKKQIQKLEARVRELEGEVESEQKRNAEAVKGLRKHERRVKELTYQTEEDRKNILRLQDLVDKLQAKVKSYKRQAEEAEEQSNTNLSKFRKLQHELEEAEERADIAESQVNKLRVKSREVHTKVISEE

SEQ ID NO: 30 - protein, 1937 aa, Sus scrofa
MSSDQEMAIFGEAAPYLRKSEKERIEAQNKPFDAKTSVFVAEPKESFVKGTVQSREGGKVTVKTEAGATLTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTPGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVQQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLIHYAGTVDYNITGWLDKNKDPLNETVVGLYQKSSVKTLAFLFAERQSSEEGGTKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGTLEEMRDEKLAQLITRTQAMCRGFLMRVEFRKMMERRESIFCIQYNIRAFMNVKHWPWMKLYFKIKPLLKSAETEKEMANMKEEFEKTKEDLAKSEAKRKELEEKMVALMQEKNDLQLQVQAEADGLADAEERCDQLIKTKIQLEAKIKEVTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARTEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLASNMETVSKAKGNLEKMCRTLEDQLSEVKTKEEEHQRLINELSAQKARLQTESGEFSRQLDEKEALVSQLSRGKQAFTQQIEELKRQLEEETKAKSALAHAVQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMLDVERSNAACAALDKKQRNFDKILAEWKHKYEETQAELEASQKESRSLSTELFKVKNAYEESLDQLETLKRENKNLQQEISDLTEQIAEGGKHIHELEKVKKQIEQEKSELQAALEEAEASLEHEEGKILRIQLELNQVKSEIDRKIAEKDEEIDQMKRNHIRVVESMQSTLDAEIRSRNDALRIKKKMEGDLNEMEIQLNHANRQATEAIRNLRNTQGVLKDTQLHLDDAIRGQDDLKEQLAMVERRANLMQAEIEELRASLEQTERSRRVAEQELLDASERVQLLHTQNTSLINTKKKLETDISQIQGEMEDIVQEARNAEEKAKKAITDAAMMAEELKKEQDTSAHLERMKKNMEQTVKDLQHRLDEAEQLALKGGKKQIQKLEARVRELENEVENEQKRNVEAVKGLRKHERRVKELTYQTEEDRKNVLRLQDLVDKLQSKVKAYKRQAEEAEEQSNVNLSKFRKLQHELEEAEERADIAESQVNKLRVKSREVHTKVISEE

SEQ ID NO: 31 - protein, 1941 aa, Gallus gallus
MATDADMAIFGEAAPYLRKSEKERIEAQNKPFDAKSSVFVVHAKESYVKSTIQSKESGHKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGDKKKEEQPAGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDYQYVSQGEITVPSINDQEELMATDSAIDILGFTPDEKTAIYKLTGAVMHYGNLKFKQKQREEQAEPGGTEVADKAAYLMGLNSADLLKALCYPRVKVGNEYVTKGQTVQQVYNSVGALAKSVFEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNITGWLEKNKDPLNETVVGLYQKSSLKTLALLFASVGGAEAESGAGGKKGGKKKGSSFQTVSALFRENLNKLMSNLRSTHPHFVRCLIPNETKTPGAMEHELVLHQLRCNGVLEGIRICRKGFPIRILYADFKQRYKVLNASAIPEGQFIDSKKASEKLLGSIDVDHTQYKFGHTKVFFKAGLLGLLEEMRDEKLAQLITRTQARCRGFLMRVEFKKMMERRESIFCIQYNVRAFMNVKHWPWMKLFFKIKPLLKSAESEKEMANMKEEFEKTKEELAKSEAKRKELEEKMVSLLQEKNDLQLQVQAEADGLADAEERCDQLIKTKIQLEAKIKELTERAEDEEEMNAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKMTQESTMDLENDKQQLDEKLKKKDFEISQIQSKIEDEQALGMQLQKKIKELQARIEELEEEIEAERTSRAKAEKHRADLSRELEEISERLEEAGGATAAQIDMNKKREAEFQKMRRDLEEATLQHEATAAALRKKHADSTADVGEQIDNLQRVKQKLEKEKSELKMEIDDLASNMESVSKAKANLEKMCRSLEDQLSEIKTKEEEQQRTINDISAQKARLQTESGEYSRQVEEKDALISQLSRGKQAFTQQIEELKRHLEEEIKAKKCPAHALQSARHDCDLLREQYEEEQEAKGELQRALSKANSEVAQWRTKYETDAIQRTEELEEAKKKLAQRLQDAEEHVEAVNSKCASLEKTKQRLQNEVEDLMIDVERSNAACAALDKKQKNFDKILSEWKQKYEETQAELEASQKESRSLSTELFKMKNAYEESLDHLETLKRENKNLQQEISDLTEQIAEGGKAIHELEKVKKQIEQEKSELQTALEEAEASLEHEEGKILRVQLELNQVKSDIDRKIAEKDEEIDQLKRNHLRVVDSMQSTLDAEIRSRNEALRLKKKMEGDLNEIEIQLSHANRQAAEAQKNLRNTQGVLKDTQIHLDDALRSQEDLKEQVAMVERRANLLQAEIEELRAALEQTERSRKVAEQELLDASERVQLLHTQNTSLINTKKKLESDISQIQSEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNVLRLQDLVDKLQMKVKSYKRQAEEAEELSNVNLSKFRKIQHELEEAEERADIAESQVNKLRAKSREIGKKAESEE

SEQ ID NO: 32 - protein, 1939 aa, Sus scrofa
MSSDQEMAIFGEAAPYLRKSEKERIEAQNKPFDAKTSVFVAEPKESFVKGTVQSREGGKVTVKTEAGATLTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVQQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKVEAHFSLIHYAGTVDYNITGWLDKNKDPLNETVVGLYQKSSVKTLAFLFTGAAGADAEAGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGLLEEMRDEKLAQLITRTQARCRGFLARVEYQKMVERRESIFCIQYNIRAFMNVKHWPWMKLYFKIKPLLKSAETEKEMANMKEEFEKTKESLAKAEAKRKELEEKMVALMQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNMETVSKAKGNLEKMCRTLEDQLSELKTKEEEQQRLINDLTAQRARLQTESGEYSRQLDEKDTLVSQLSRGKQAFTQQIEELKRQLEEEIKAKSALAHAVQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMIDVERSNAACAALDKKQRNFDKILAEWKQKYEETHAELEASQKESRSLSTELFKVKNAYEESLDQLETLKRENKNLQQEISDLTEQIAEGGKRIHELEKIKKQVEQEKSEIQAALEEAEASLEHEEGKILRIQLELNQVKSEVDRKIAEKDEEIDQLKRNHVRVVESMQSMLDAEIRSRNDAIRLKKKMEGDLNEMEIQLNHANRMAAEALRNYRNTQGILKDTQIHLDDALRSQEDLKEQLAMVERRANLLQAEIEELRATLEQTERSRKVAEQELLDASERVQLLHTQNTSLINTKKKLETDISQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 33 - protein, 1941 aa, Gallus gallus
MASSDAEMAAFGEAAPYHRKSEKERIEAQNKPFDAKSSVFVAHPKESFVKGTIQSRETGKVTVKTEGGETLTVKEDQVFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQSGKMQGTLEDQIISANPLLEAFGNAETVRNDNSSRFGKFIRIHFGATGKLASADIETYLLEKSRVTFQLKAERSYHIFYQIMSNKKPELIDMLLITTNPYDYHFVSQGEITVPSIDDQEELMATDSAIDILGFTADEKTAISKLTGAVMHYGNLKFKQKQREEQAEPDGTEVADKAAYLMGLNSADLLKALCYPRVKVGNEYVTKGQTVQQVHNAVGALAKAVYEKMFLWMVVRINQQLDTKQPRQYFIGVLDIAGFEIFDFNSFEQLCINFTNEKLQQFFNHHMFVLEQEEYKKEGIEWTFIDFGMDLAACIELIEKPMGIFSILEEECMFPKATDTSFKNKLYDQHLGKSSNFQKPKPAKGKAEAHFSLVHYAGTVDYNITGWLEKNKDPLNETVIGLYQKSSVKTLALLFATYGGADAEAGGGGKKGGKKKGSSFQTVSALFRENLNKLMTNLRSTHPHFVRCIIPNETKTPGAMEHELVLHQLRCNGVLEGIRICRKGFPSRVLYADFKQRYKVLNASAIPEGQFIDSKKASEKLLSSIDVDHTQYKFGHTKVFFKAGLLGLLEEMRDEKLAQLITRTQARSRGFLMRVEYQRMVERRESIFCIQYNVRSFMNVKHWPWMKLFFKIKPLLKSAESEKEMANMKEEFEKTKEELAKSEAKRKELEEKMVKLVQEKNDLQLQVQAEADSLADAEERCDQLIKTKIQLEAKIKEVTERAEDEEEINAELTAKKRKLEDECSELKKDMDDLELTLAKVEKEKHATENKVKNLTEEMAALDETIVKLTKEKKALQEAHQQTLDDLQAEEDKVNTLTKAKTKLEQQVDDLEGSLEQEKKLRMDLERAKRKLEGDLKLAHDSIMDLENDKQQLDEKLKKKDFEISQIQSKIEDEQALGMQLQKKIKELQARIEELEEEIEAERTSRAKAEKHRADLSRELEEISERLEEAGGATAAQIDMNKKREAEFQKMRRDLEEATLQHEATAAALRKKHADSTAELGEQIDNLQRVKQKLEKEKSELKMEIDDLASNMESVSKAKANLEKMCRTLEDQLSEIKTKEEEHQRMINDLNTQRARLQTEAGEYSRQVEEKDALISQLSRGKQAFTQQIEELKRHLEEEIKAKNALAHALQSARHDCDLLREQYEEEQEAKGELQRALSKANSEVAQWRTKYETDAIQRTEELEEAKKKLAQRLQDAEEHVEAVNAKCASLEKTKQRLQNEVEDLMIDVERANAACAALDKKQKNFDKILAEWKQKYEETQAELEASQKESRSLSTELFKMKNAYEESLDHLQTLKRENKNLQQEISDLTEQIAEGGKAIHELEKVKKQIEQEKSEIQAALEEAEASLEHEEGKILRLQLELNQVKSEIDRKIAEKDEEIDQLKRNHLRIVESLQSSLDAEIRSRNEALRLKKKMEGDLNEMEIQLSHANRVAAEAQKNLRNTQAVLKDTQIHLDDALRTQEVLKEQVAMVERRANLLQAEIEELRAALEQTERSRKVAEQELMDASERVQLLHTQNTSLINTKKKLETDIAQIQSEMEDTIQEARNTEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDLKNILRLQDLVDKLQMKVKSYKRQAEEAEELSNVNLSKFRKIQHELEEAEERADIAESQVNKLRVKSREFHSKKIEEEE

SEQ ID NO: 34 - protein, 1939 aa, Gallus gallus; identical to SEQ ID NO: 13

SEQ ID NO: 35 - protein, 1132 aa, Gallus gallus
MPEPSKAAPKKEAKKKEEKKEEKKEAPPPQEHKDEAPDDVHPPETPDPEGLFLSKPQNVMVESGRDVTVSARVAGAALPCAPAVKWFKGKWAELGDKSARCRLRHSVDDDKVHTFELTITKVAMGDRGDYRCEVTAKEQKDSCSFSIDVEAPRSSEGNVLQAFKRTGEGKDDTAGELDFSGLLKKREVQVEEKKKKKDEDDQFPPEIWELLKGVTKKSEYERIAFQYGITDLRGMLKRLKKVHVEPKKSEAFIRKLDPAYQVDKGNKIKLVVELSDPDLPLKWYKNGQLLKPSTKYVFENVGLKRILTIHKCSLADDAAYECRVNDEKCFTEVFVKEPPVTVVRGLEDQQVVVGDRVVLEAEVSEEGAQVMWLKDGVDVTRDDAFKYRFKKDGKKHFLIINEAELSDSAHYKIMTNGGESEAELSVEEKQLEVLQDMADLTVKASEQAVFKCEVSDEKVTGRWFRNGVEVKPSKRIHISHNGRFHKLVIDDVRPEDEGDYTFIPDGYALSLSAKLNFLEIKVEYVPKQEPPKIHLDCSGKAAENTIVVVAGNKVRLDVPISGEPAPTVTWKRGDQLFTATEGRVHIDSQADLSSFVIESAERSDEGRYCITVTNPVGEDSATLHVRVVDVPDPPQSVRVTSVGEDWAVLSWEAPPFDGGMPITGYLMERKKKGSMRWMKLNFEVFPDTTYESTKMIEGVFYEMRVFAVNAIGVSQPSLNTQPFMPIAPTSEPTHVVLEDVTDTTATIKWRPPERIGAGGVDGYLVEWCREGSNEWVAANTELVERCGLTARGLPTGERLLFRVISVNMAGKSPPATMAQPVTIREIVERPKIRLPRHLRQTYIRRVGEQVNLVIPFQGKPRPQVTWSREGGALPAEVQTRTSDVDSVFFIRSAARPLSGNYEMRVRIDNMEDCATLRLRVVERPGPPQAVRVMEVWGSNALLQWEPPKDDGNAEISGYTVQKADTRTMEWFTVLEHSRPTRCTVSELVMGNEYRFRVYSENVCGTSQEPATSHNTARIAKEGLTLKMVPYKERDLRAAPQFLTPLVDRSVVAGYTVTLNCAVRGHPKPKVTWLKNSVEIGADPKFLSRHGLGVLSLLIRRPGPFDGGTYGCRAVNEMGEATTECRLDVRVPQ

SEQ ID NO: 36 - protein, 1135 aa, Bos taurus
MPEAKPAAKKAPAGKDAAAKPAPKEATPQEAPAAPPTEAPPEDQSPTAEEPTGVFLKKPDSVSVETGKDTVIVAKLNGKELPAKPAVKWFKGKWLELGSKSGARFSFKESHDAASNVYTIELHITKVVLGDRGDYRIEVKAKDFCDSCAFNIDVEAPRQDSAGQSLESFKRSGEAKSDTAGELDFSGLLKKRQVVEEEKKKKKDDDDLGIPPEIWELLKGAKKSEYERIAFQYGITDLRGMLKRLKKAKVEVKKSAAFTKKLDPAYQVDRGNKIKLVVEISDPDLPLKWFKNGQEIKPSSKYVFENVGKKRILTINKCTLADDAAYEVVVKDEKCFTELFVKVEPPVLIVTPLEDQQVFVGDRVEMAVEVSEEGAQVMWLKDGVELTREDSFKARYRLKKDGKRHILIYSEVTMEDKGHYQVMTNGGQCEAELIVEEKQLEVLQDIADLTVKASEQAVFKCEVSDEKVTGKWYKNGVEVRPSKRITISHTGRFHKLVIDDVRPEDEGDYTFVPDGYALSLSAKLKFLEIKVEYVPKQEPPKIHLDCSGQTSENAIVVVAGNKLRMDVSITGEPRPVATWMKEDEVFTGTEGRVRIEQRGDISSFVIESAERGDEGRYTIKVTNPVGEDVASILLKVVDVPDPPEAVRVTSVGEDWAVLVWEPPKYDGGRPVTGYLLERKKKGSQRWMKLNFEVFTETTYESTNMIEGILYEMRVFAAPLIQQLKTLTPQPHAPFSPTSEPQHLTVEDVTDTTTTLKWRPPDKIGAGGIDGYLIEYCVEGSDEWIPANTEPVERCGFTVKNLPTGAKITFRVVGVNIAGRSQPATLAQPVTIREIVEQPKIRLPRHLRQTYVRKVGEHLNLVIPFQVRRAKGGAPIDTSHVHVRTSDFDTVFFVRQAARSDSGEYELTVQIENMKDTATVCIRVVEKAGPPQNVMVKEVWGTNALVEWQPPKDDGNSEITGYFVQKADKKTMEWFTVYERNRHTSCTVSDLIMGNEYYFRVYSENVCGLSDLPGVSKNTARIVKTGMTLKLPEYKEHDFRTPPKFLTPLPDRVVVAGYAAALNCAVRGHPKPKVVWMKNKMEIREDPKFLMTNQQGVLTLNIRRPSPFDSGTYSCRAVNELGEALAECKLEVRAPR

SEQ ID NO: 37 - protein, 537 aa, Gallus gallus
MTGKTAPAAAKKAPAAKKAPAPASKKAPEPAPKEKPAPTPKEGHAPTPKEEHAPPPKEEHAPPPKEEHAPAPAAETPPAPEHPPDAEQPAAPAAEHAPTPTHEAAPAHEEGPPPAAPAEAPAPEPEPEKPKEEPPSVPLSLAVEEVTENSVTLTWKAPEHTGKSSLDGYVVEICKDGSTDWTAVNKEPFLSTRYKIHDLASGEKVHVRVKAISASGTSDPATLEQPVLIREITDLPRIRLPRQLRQVYVRHVGEAVNLLIPFQGKPQPQVTWTKDNQPLDTSRVNIRNTDKDTIFFIRTAQRSDSGKYQLSVRINGAEDKAILDIRVIERPGPPQNLKLVDVWGFNVALEWSPPADNGNSEIKGYTVQKSDKKSGKWFTVLERCTRTSCTISDLIIGNTYSFRVFSENACGMSETAAVAAGVAHIKKTVYQPQKIPERDMMEPPKFTQPLTDRATTRGYSTHLFCSVRGFPQPKIIWMKNKMEIREDPKYIAMIEQGVCSLEIRKPSPFDAGVYTCKAVNPLGEASVDCKLDVKMPK

SEQ ID NO: 38 - protein, 491 aa, Bos taurus
MTEKATSEAPACGLEETTSESAHVPLTEPSGDTAAPQAPGGEQAPRGQQASDPQESARQPPDPAASAAPAGPAATDPALPREDVPSAPLLLAVEDVSDSSVTVSWEPPERLGRLGLQGYVLELRREGALDWVPVNARPMMVTQQTLRNLAVGDKFFVRVAAVSSAGAGPPAVLERLIHIQETIEAPKIRVPRHLRQTYIRQVGESINLQIPFQGNPKPQALWTHNGHALDSQRVSVRTGDQDSILFIRSAQRSDSGCYELTVQLKDLEAKAAINILVIEKPGPPRSIRLLDVWNCNATLEWTPPQDTGNTELLGYTVQKADKKTGQWFTVLERCHPTSCTVSDLIVGNSYSFRVFSENLCGLSASAAVTKELAHIVKTDIVAKPKSFVERDFSEAPSFTQPLADHTSTPGYSTQLSCSVRASPKPKIIWMKNKMDIQGDPKYRALSEQGVCTLEIRKPSPFDSGVYTCKAINVLGEASVDCRLEVKASATH

SEQ ID NO: 39 - protein, 8074 aa, Gallus gallus
MTTKAPTFTQPLQSVVALEGSAATFEAHISGFPVPEVSWYRDGQVLSAATLPGVQISFSDGRAKLVIPSVTEANSGRYTIQATNGSGQATSTAELLVTAGTAPPNFSQRLQSMTARQGSQVRLDVRVTGIPTPVVKFYRDGVEIQSSPDFQILQEGDLYSLIIAEAYPEDSGTYSVNATNNVGRATSTAELLIQGEEEAVPAKKTKTIVSTAQISQTRQARIEKKIETHFDARSLTSVEMVIEGAAAQQLPHKAPPRMPPRPTSKSPTPPVITAKAQMARQQSPSPVRQSPSPVRHVRAPTPSPVRSVSPAGRISTSPIRPVKSPSPIRKAQVVTPGAEVLPPWRQEGYSATAEAQMKETRVSTSATEIRTEERWEGRYGLQEQVTISGAAAGEVAAGAKEVRKEPEKTPVPTVIIATDKAKEQERISTAREEISARHEQVHVSHEQIEAGKRAEAVATVVAAVDQARVRSPWETEQVDETYVKKKTLEYGYKEHAVKDHEAQAEHHVATKEVKTVYVPPEKHIPAAEKKEVHVSTEIKRETEAKIEKTIHIEHPRPRTASPHFTVSKIAVPKPDHTYEVSIAGSAMATLEKELSATSAAQKITKPVKPPQLKPHEVKIKPESAPPQFPFTEAAETYKAHYDVETKKEVDVSIKGEAVREDHLLLRKESEAKVTETARVPVPAEIPVTPPTLVWGLKNKTVTEGESVTLECHISGHPQPTVTWYREDYKIESSMDFQITFKAGLARLVIREAFAEDSGRFTCTATNKAGSVSTSCHLHVKVSEETETRETISEKVVTEEKSYVETKDVVMEDVSAAAEEVSGEPVPPFFIRKPVVHKLIEGGSIIFECQVGGNPKPHVLWKKGGVPLTTGYRYKVSYKRETGECKLEISMTFADDAGEYTIVIRNKFGEASATVSLLEEADYEAYIKSQQEMMYQTQVTAYVQEPKVAEVAPPISYGDFDKEYEKEQALIRKKMAKDTVMVRTFVEDEEFHISSFEERLIKEIELRIIKTTLDELLEEDGEEMMIDISESEAIGAGFDLRLKNYRTFEGTGVTFHCKTTGYPLPKIAWYKDGKRIRHGERYHMEVLQDGSASLRLPVVLPEDEGIYTVFASNMKGNAICSAKLYVEPVAPTATPGYMPGPEVMRRYRSISPRSPSRSPARSSPSCSPARRLDETDEGQLERLYKPVFVLKPTSVKCSQGQTARFDLKVVGRPMPETYWFHNGQQVVNDYTHKIVIKEDGTQSLIIVPAMPEDSGEWAVIAQNRAGKASVSVTLSVEAKEDLVRPRFVERLRNVSVKEGSRLHMAVKATGNPNPDIVWLKNSDIIVPHKYPRIRIEGTKGAAALNIESTARQDAAWYTATAINKAGRDTTRCKVNVEVEHAEPEPERRLIIPKGTYKAKEIAAPELEPLHLRYGQEQWEEGDLYDKEKQQKPFFKKKLTSLRLKQFGPAHFECRLTPIGDPTMVVEWLHDGKPLEAANRLRMINEFGYCSLDYGVAYSRDSGVITCRATNKYGTDHTSATLIVKDEKSLVEESQLPEGRRGMQRIEELERMAHEGALPAVAVDQKEKQKPELVLVPEPARVLEGETARFRCRVTGYPLPKVNWYLNSQLIRKSKRFRLRYDGIHYLDIVDCKSYDTGEVKVTAENPEGFIEHKVKLEIQQREDFRSVLRRAPEPRHEPVVTEPGKLLFEVQKIDKPAEATTKEVVKLKRAERITHEKLSEESEELRSKFKRRTEEGYYEAITAVELKSRKKDESYEEMLKKTKEELLHWTKEIPEEEKKALPPEGKITIPTFKPEKVELSPSMEAPKIFERIQSQTVAQGTDAHFRVRVVGKPDPECQWFRNGVQIERTDRIYWYWPEDNVCELVIRDVTADDSASIMVKAVNIAGETSSHAFLLVQAKQLISFIQNLQDVVAKERDSMATFECETSEPFIKVKWFKNGIEIHSGEKYRMHSDRKAHFLSVLAVEMSDADDYSCALVEDESVKTTAKLIVEGAVVEFIKELEDVEVPESFTGELECEVSPEDIEGKWYHGDVELSSNHKYVLASRRGRRILTIKDVNKDDQGEYSFVVDGKRTHCKLKMKPRPMTILQGLTDQKVCEGDIVQLEVKVSVENVEGVWMKDGHEIQSSDRIHIVLDKQSHMLLIEDATQEDSGTYSFSIPGLELSTTGQVTVYSVEIIVPLKDVHVVEGTKAILECKVSAPDVTSSKWYLNDHQIKPDERVQAVCKGTKQRLVITRTHASDEGHYKLMVGKVETSCNVTVEEIEIIRGLHDITCTETQNVSFEVELSHSGIDVIWHFKGQEIKAGPKYKIEARGKIYKLTVVKMMKDDEGEYVFYAGGKKTSGKLIVAGGAISKPLADLTVAESQRAVFECEVANPESEGQWLKNGKPLPMTDQYRAETDGVKRRLNIPAAKMDDMGEYSYEIASSKTSAKLHVEAVKIKKTLKNLTVTETQEAVFSVELSHPDVKGALWIKNGVELESNDKYEISVKGTVHTLKIKHCVVTDESVYSFKLGKIGANARLHVETVKIIKKPKDVTALENAVVSFELSVSHDTVPVRWFHKNVELKQSDKYKMISQRKVHKLMLHNISPADAGEYTAFVGQLECKAKLFVETIHITKTMKSIEIPETKTASFQCEVSHFNVPSVWLKNGVEIEMSEKFKIVVQGKLHQLNIMNTSSEDSAEYTFVCGNDRVSATLTVKPILITSMLEDINAEEKDTITFEVTVNYEGISYKWLKNGVEIKSTDKCQIRTKKLTHSLSIRNVHFGDAAEYSFVAGKAASSATLYVEARHIEFRKHIKDIKVVEKKRAIFECEISEPDVQVQWMKDGQELQIGDRMKIQREKYVHRLIIPSTKMSDAGQYTVVAGGNTSSANLIVEGRDVRIRSIRKEIQVIERQRAEIEFEVNEDDIEPQWYKDGIEINFHYEERYSYVVERRIHRMSIFETTYSDAGEYTFVAGRNRSSVVLYVNAPEPPQIIQELQPTTVESGKPARFCAIISGKPQPKVSWYKDDQQLSPGFKCKFLHDAQEYTLLLIETFPEDSAVYTCEAKNDYGVATTSASLSVEIPEVVSPELEVPVYPPAVIVPLRDAVTSEGQSARFQCRVTGTDLKVSWYSKDREIKPSRFFRMTQFEDTYQLEIAEAYPEDEGTYTFVASNSVGQVTSTAILKLEAPEKIMYEKLEEEIEMEVKVAPILRRRLEPLEVAVNHVAKFTCEVETTPNVKFQWYKAGREIYDGDKYSIRSSNYLSTLEIPRPQVVDCGEYSCKASNQHGSVSSTAFLTVTEPPRFIKKLDSSRLVKQHDSTRYECKVGGSPEIKVTWYKGETEIHPSEKYSMSFVDSVAVLEMHNLSVEDSGDYSCEAQNPAGSASTSTSLKVKAPPAFTKKPHPVQTLKGSDVHLECELQGTPPFQISWYKDKREIRSSKKYKVMSENYLASIHILNVDTADVGEYHCKAVNDVGSDSCIGSVTLRAPPTFVKKLSDVTVVVGETIELQAAVEGAQPISVLWLKDKGEIIRESENLWISYSENVASLKIGNAEPTNAGKYICQIKNDAGFQECFAKLTVLEPAVIVEKPGPVKVTAGDSCTLECTVDGTPELTARWFKDGNELSTDHKYKISFFNKVSGLKILNAGLEDSGEYTFEVKNSVGKSSCTASLQVSDRIMPPSFTRKLKETYGQLGSSAVLECKVYGSPPILVSWFHDGQEITSGDKYQATLTDNTCSLKVNGLQESDMGTYSCTATNVAGSDECSAFLSVREPPSFVKKPEPFNVLSGENITFTSIVKGSPPLEVKWFRGSIELAPGHKCNITLQDSVAELELFDVQPLQSGDYTCQVSNEAGKISCTTHLFVKEPAKFVMKVNDLSVEKGKNLILECTYTGTPPISVTWKKNGVILKHSEKCSITTTETSAILEIPNSKLEDQGQYSCHIENDSGQDNCHGAITILEPPYFVTPLEPVQVTVGDSASLQCQVAGTPEMIVSWYKGDTKLRGTATVKMHFKNQVATLVFSQVDSDDSGEYICKVENTVGEATSSSLLTVQERKLPPSFTRKLRDVHETVGLPVTFDCGIAGSEPIEVSWFKDNVRVKEDYNVHTSFIDNVAILQILKTDKSLMGQYTCTASNAIGTASSSGKLVLTEGKTPPFFDTPITPVDGIIGESADFECHISGTQPIRVTWAKDNQEIRTGGNYQISYVENTAHLTILRVDRGDSGKYTCYASNEVGKDSCTAQLNVKERKTPPTFTRKLSEAVEETEGNELKLEGRVAGSQPLTVSWYKNNQEVHSSPHCEISFKNNTLLLHIKSVGQSDAGLYTCKVSNEAGSVLCTSSVVIREPKKPPVFDQPLQPAATEEGDTLQLSCHVRGSEPIRIQWLKAGREIRASERCSFSFANGVALLELAAVTKSDSGEYVCKASNVAGTDTCRSKVTVKEKAALVSAAKKADIEGKLYFVSEPQSIKVMEKTVATFIAKVGGDPIPNVKWMKGKWRQLNQGGRIIIQQRGDEAKLEIKDTTKTDSGLYKCVAFNQHGEIERSVNLQVEERKQEVVEEDVRGKLKRIPTKKKEDEEQTIDILELLKNVDPKEYEKYARMYGITDFRGLLQAFELLKQTREEESHRLEIELTEKAQKEDQEFEELVAFIQQRLTQTEPVTLIRDIENQTVLTDEDAIFECEIKINYPEIKLSWYKGTQKLDSSDKYKIKIEGDRHILKIKNCQLEDQGNYRIVCGPHIASARLTVIEPAVERHLHDTTFKEGNTCTLSCQFSIPNAKSQWYRNGRPIKIGGRYSTQVSDKVHKLIIKDVRTEDQGQYTCKLDNLETTADLTIEAEPIQFTKSIQNIVVSEHQSATFECEVSFDDAVVTWYKGPTELTESPKYSFRSEGRCHYMTIHNVTAEDEGVYSVIARLEPRGEARSTAELYLVTKEIKLELKPPDVPDAKVAVPPQKPAEAAPIPILLPLIPTPEEKKPAEKKVPVKKVSKKVVKKGPKEIPPPEVPEILPEKPKEVKITSMARREEIHEEKMEIYEKPKKVYEEWEEDYGEDHDYYFKEEGYDEGEEEWEETYDKREVAYEEEQIIHEEVVEVPKKPVPERKPPAITAEKKEKKEVTVHKVPDVPKKIPEEKVPITVPKKPEPTPAKEPEVHKIIEEKIKVSEPKKPEVPPAKVPEVPKKVVKEEVSVEVPKKPEPPPAKVPEVPKKVVPEEKVHVEVPKKAEPPPPKVLRKKPEEEEPKPEKEPKPEVKPVPTPVEKIKKPEVPEVPKKKTEIPVIKKEEKHVPEPPKEPKPAAISVPGPEPKPAVALKSPKPAAEPAPAITTAPVTTPVVGKKAEAKPTKEETPKGISPVKAKKTPSPADAERRKLRPGSGGEKPPEEPPFTYQLKAVPLKFVKEMKDIVLKEAESVGSSAIFEVLISPSTAITSWMKDGSNIRESPKHKFIADGKDRKLHIIDVQLSDAGEYTCVLRLGNKEKTSTAKLIVEELPVRFVKTLEEEVTVVKGQPLYLTCELNKERSVVWRRDGKIIKDKPGKFALGIIGLSHSLTITDSDDSDAGTYTVTVEDSELSCSSCVKVVEVIRDWLVKPIRDQHVKPKGTATFTCDIVKDTPNIKWFKGDEEIPAEPTDKTEILKEGNKIFLKIKNAGPADIGEYSVEVEGRRYPAKLTLGEREVELLKPLEDVTVYEKETANFDTEISEEDIPGEWKLKGEILRPSPTCEIKAEGGKRFLTLHKVKLEQAGEVLYQALNAVTTAILTVKEIELDFAVPLKDVTVPEKRQARFECVLTREANVIWSKGTDILKIGEKFDIIADGKKHILVINDSQFDDEGEYTAEVEGKKSTARLFVEGVRLKFITPLQDQTVKEGETAYFQFELSHEGMLVKWYKNDKRLHTSRTVFITSEGKVHKLEMREVTLDDISEIKAVVKDMNTQANLKVLEADPYFTVKLQDYSALEKDDITLQCELSKDVPVKWYKDGEELVASSRISIKTDGLRRILTIKKATEGDKGVYECSCGTDKTNCNIGVEPRLIKVERPLYGVEVFVGETARFEIEISEPDVHPIWKLKGETLTPSPECEIIEDGKKHILILHQGRLDMTGEVSFQAANAKSAANLKVKELPLIFITPLSDVKVFEKDEAKFECEVSREPKTFRWLKGTQEITPDERFEIISDGTKHALIIKSVAFDDEAKYMFEAEDKRTSAKLIIEGIRLKFITPLKDVTKKERETAVFTVELSHENIPVVWFKNDQRLHTSKVVSMTDDGKFHTLTIKDLTIDDTSQIRVEAMGKSSEAKLTVLEGDPYFTGKLQDYTAVEKDEVILQCEISKADAPVKWMKDGKPITPSKNVVIKADGKKRILILKKALKKDIGQYTCDCGTDQTSANLNIEDRDIEIIRPLYSVEVIETETARFDIEISEEGVHGNWKLKGEPLTESPDCEIKEEGKKHFLTLYNVRLDQAGGVDFQAANAKSGAHLRVKPRVIGLLRPLKDVTVTAGESATFDCELSYEGIPVEWYLQGKKLEPSDKVVTRAEGRAHTLILRDVKLTDAGEVSLTAKDFRTQANLFVKEPPVEFTKPLEDQTVEEEATAILECEVSRENAKVKWFKNGEEIHKTKKYDIISEGRVRKLIIHGCTLDDAKTYTCDAKDFKTSCFLNVEPLRVEFLRPLTDLEVKEKRICSVLNVKFLAQGVKVKWFKDGIEIEKAEYDIISKGAERILVISKCLFDDEAEYACEAKTARTSGLLTVIEEEAVFTKNLHDLEVNENETVRLICEISKPNAEVTWFKGDEEVPDGGRFEYISDGRKRILVIRNAHPEDAGKYTCKLPSSSTTGKLTVHELAAEFLTRPQNLEVLEGDKAEFACSVSKEAITVQWLWGDTVLEPGDKYDIISDGKKRTLVVKDSVVGDAGKYTVMVGEAKATARLTVIEKTQDYNSLKDQEVNEGQEIIFNCEVNKEGAKEKWYKNGEAIFDSAKYIIVQKDLVYTLRIRDTQLKDQATYSISLSNHRGEHAESSAALTVLEEGLRIVEPLEDIETMEKKTVTFWCKVNRLNATLKWTKNGEEITFNKRILYKIDKYKHSLIIKDCGFKDEGEYTVTAGQDKSVAELLITEAPADFIEHLQDQTVTEFDDAVFTCQLSKEKASVKWYRNGREIKEGKKYQFEKDGNLHRLIIKDCRLDDECEYSCGVDDRKSRARLFVEEIPVEIIRPPQDVYEAPGADVIFMAELNKDGVEVKWLRNNMIIIQGDKHQMMSEGKVHRLQVCEIKPRDQGEYRFIAKDKEARAKLELAAAPRIKTGDQNLVVDVGNPLTMTVPYDAYPRAEAEWLKGEESLPTTTVDTTTDCTTFKIYEAKKSDKGRYKVVLRNKHAQAEAFINVEVIDVPGPVRNLEVTEIYDGEVGLAWQEPESDGGSKIIGYVVERRDIKRKTWIVVTDHAENCEYTVTGLQKGGVEYLFRVSARNRVGTGEPVETERPVEAKSKFEVPGPPQNVEVTDVNRFGRTLTWEAPEYDGGSPITGYVIELRNRASIKWEPTMTTGADELSAVLTDVVENEEYFFRVRAQNMVGVGKPSHPTRAVKITDPIERPSGNINLDHSDQTKTSVQLTWEPPLEDGGSPILGYIIERKEEGTDKWIRCNPKLVPACAFKVTGLKAGSSYYYRVSAENAAGVSDPAEAIGPLTADDPFVDLQWPLSAFKDGLEVIVPEPIKIRVPITGYPIPTATWSFGDQVLEEGGRVTMKTTSTFAELVITPSERPDKGIYTLTLENPVSSVSGEIDVNVLARPSAPKELKVVDVSRSTVQLSWEPPEDDGGSPVIDYIIEKREVSRKTWIKVMDHVIDQEFSVPDLIQGKEYLFRVCACNKCGPGEPAYIDEPINMSAPATVPDPPENVKWRNPTSKGIFLTWEPPKYDGGARIKGYLVEKCQRGTDKWEICGEPVIETKMEVTKLKEGEWYAYRVKALNRIGASKPSKPTDDIQAIDAKEAPEIFLDVKLLAGLTVKAGTKIELPAKITGKPEPQITWTKAEKLLRPDDRITIETTPNHSTVTITDSKRSDSGTYIIEAVNSSGRATAVVEVNVLDKPGPPAAFDVSEITNESCLLTWNPPRDDGGSKITNYVLEKRATDSEIWHKLSSTI

SEQ ID NO: 40 - protein, 539 aa, Gallus gallus
EGEEEWEETYDKREVAYEEEQIIHEEVVEVPKKPVPERKPPAITAEKKEKKEVTVHKVVKKPVDEKVEITTQRVAEEKLKQAEVTKKIPSAKPTPMIIEEKVMKKEAHKEVEEEEEREVISEELETHHVEVPDVPKKIPEEKVPITVPKKPEPTPAKEPEVHKIIEEKIKVSEPKKPEVPPAKVPEVPKKVVKEEVSVEVPKKPEPPPAKVPEVPKKVVPEEKVHVEVPKKAEPPPPKVLRKKPEEEEPKPEKEPKPEVKPVPTPVEKIKKPEVPEVPKKKTEIPVIKKEEKHVPEPPKEPKPAAISVPGPEPKPAVALKSPKPAAEPAPAITTAPVTTPVVGKKAEAKPTKEETPKGISPVKAKKTPSPADAERRKLRPGSGGEKPPEEPPFTYQLKAVPLKFVKEMKDIVLKEAESVGSSAIFEVLISPSTAITSWMKDGSNIRESPKHKFIADGKDRKLHIIDVQLSDAGEYTCVLRLGNKEKTSTAKLIVEELPVRFVKTLEEEVTVVKGQPLYLTCELNKERSVVWRRDGKIIK

SEQ ID NO: 41 - protein, 989 aa, Gallus gallus
SDKYKIKIEGDRHILKIKNCQLEDQGNYRIVCGPHIASARLTVIAEPIQFTKSIQNIVVSEHQSATFECEVSFDDAVVTWYKGPTELTESPKYSFRSEGRCHYMTIHNVTAEDEGVYSVIARLEPRGEARSTAELYLVTKEIKLELKPPDVPDAKVAVPPQKPAEAAPIPILLPLIPTPEEKKPAEKKVPVKKVSKKVVKKGPKEIPPPEVPEILPEKPKEVKITSMARREEIHEEKMEIYEKPKKVYEEWEEDYGEDHDYYFKEEGYDEGEEEWEETYDKREVAYEEEQIIHEEVVEVPKKPVPERKPPAITAEKKEKKEVTVHKVPDVPKKIPEEKVPITVPKKPEPTPAKEPEVHKIIEEKIKVSEPKKPEVPPAKVPEVPKKVVKEEVSVEVPKKPEPPPAKVPEVPKKVVPEEKVHVEVPKKAEPPPPKVLRKKPEEEEPKPEKEPKPEVKPVPTPVEKIKKPEVPEVPKKKTEIPVIKKEEKHVPEPPKEPKPAAISVPGPEPKPAVALKSPKPAAEPAPAITTAPVTTPVVGKKAEAKPTKEETPKGISPVKAKKTPSPADAERRKLRPGSGGEKPPEEPPFTYQLKAVPLKFVKEMKDIVLKEAESVGSSAIFEVLISPSTAITSWMKDGSNIRESPKHKFIADGKDRKLHIIDVQLSDAGEYTCVLRLGNKEKTSTAKLIVEELPVRFVKTLEEEVTVVKGQPLYLTCELNKERSVVWRRDGKIIKDKPGKFALGIIGLSHSLTITDSDDSDAGTYTVTVEDSELSCSSCVKVVEVIRDWLVKPIRDQHVKPKGTATFTCDIVKDTPNIKWFKGDEEIPAEPTDKTEILKEGNKIFLKIKNAGPADIGEYSVEVEGRRYPAKLTLGEREVELLKPLEDVTVYEKETANFDTEISEEDIPGEWKLKGEILRPSPTCEIKAEGGKRFLTLHKVKLEQAGEVLYQALNAVTTAILTVKEIELDFAVPLKDVTVPEKRQARFEC

SEQ ID NO: 42 - protein, 1020 aa, Gallus gallus
KSRLRRRREREITEITSEEEEEIEMVQHVHREFSPPSRLLRRRRSLSPTYIELMRPVSELIRPRSRPPEESERRSPTPERTRPRSPSPVSTERSLSRFERMARFDIFSRYESMKSALKTQKTMERKYEVLTQQPFTLDHAPRITLRMRSHRVPCGHNTRFILNVQSKPTADVKWYHNGIELQESSKIHFSNTSGVLTLEILDCHIDDSGTYRAVCTNYKGECSDYATLDVTGGDYTTYSSQRRDEEVPRSILPDLTRTEAYAVSSFKKATAAEASSSVREVKSEVSATRESLLSYEHHASSEEKITASEEKSLEERTVHKAFKSTLPATILTKPRSITVSEGETARFSCDVDGEPAPTITWVRAGQPIVSSRRFQITRTQYKSTFEISLVQIADEGSYTVVVENSEGRQEAHFTLTVQRKRIPEKAITSPPRIKSPEPRVKSPEPVKSPKRVKSPEPISTPSKAKSPPGDKTAPVEKVQLPTASPPKIKEQLKAETLGDKVKLSCAVESSVLSIREVAWYKDGKKLKEDHHFKFHYAADGTYELKIHNLTESDKGEYTCEIMGEGGISKTNFQFTGQVFKNIHSQVQSVSETPKSVEKGDKVLAVSTQKKSSAATEEKAAIEEVIKKSIVTEDVKQLQAEIRASSTQMTVSEGQKVTLKANIPGASEVKWVLNGMELRNSDDYRYGISGSNHTLTIKKASNKDEGILTCEGKTDEGTIKCQYVLTFSKEPSNEPAFITQPKSQNVNEGQDVLFTCEVSGDPSPEVEWLRNNQPIAVSSHMRATRSKNTYSLEIRNAAVSDTGKYTVKAKNYHGQCSATASLTVFPLIEEPPKEVVLKTSGDASMHESFSSQSFQMAASKQEASFSSFSSSSMTETKFASMSAKSMSSMKESFVEMSSSSIMGKSSMAQLESSTSKMLKSGVRGVPPKIEALPSDISIDEGKVLTLSCAFSGEPAPEITWYCRGRKITSQDQQGRFHIETSEDLTTLIIMDVQKNDGGIYTLNLGNEFGTDSATVNINIRS

SEQ ID NO: 43 - protein, 4162 aa, Gallus gallus
MTTKAPTFTQPLQSVVALEGSAATFEAHISGFPVPEVSWYRDGQVLSAATLPGVQISFSDGRAKLVIPSVTEANSGRYTIQATNGSGQATSTAELLVTAGTAPPNFSQRLQSMTARQGSQVRLDVRVTGIPTPVVKFYRDGVEIQSSPDFQILQEGDLYSLIIAEAYPEDSGTYSVNATNNVGRATSTAELLIQGEEEAVPAKKTKTIVSTAQISQTRQARIEKKIETHFDARSLTSVEMVIEGAAAQQLPHKAPPRMPPRPTSKSPTPPVITAKAQMARQQSPSPVRQSPSPVRHVRAPTPSPVRSVSPAGRISTSPIRPVKSPSPIRKAQVVTPGAEVLPPWRQEGYSATAEAQMKETRVSTSATEIRTEERWEGRYGLQEQVTISGAAAGEVAAGAKEVRKEPEKTPVPTVIIATDKAKEQERISTAREEISARHEQVHVSHEQIEAGKRAEAVATVVAAVDQARVRSPWETEQVDETYVKKKTLEYGYKEHAVKDHEAQAEHHVATKEVKTVYVPPEKHIPAAEKKEVHVSTEIKRETEAKIEKTIHIEHPRPRTASPHFTVSKIAVPKPDHTYEVSIAGSAMATLEKELSATSAAQKITKPVKPPQLKPHEVKIKPESAPPQFPFTEAAETYKAHYDVETKKEVDVSIKGEAVREDHLLLRKESEAKVTETARVPVPAEIPVTPPTLVWGLKNKTVTEGESVTLECHISGHPQPTVTWYREDYKIESSMDFQITFKAGLARLVIREAFAEDSGRFTCTATNKAGSVSTSCHLHVKVSEETETRETISEKVVTEEKSYVETKDVVMEDVSAAAEEVSGEPVPPFFIRKPVVHKLIEGGSIIFECQVGGNPKPHVLWKKGGVPLTTGYRYKVSYKRETGECKLEISMTFADDAGEYTIVIRNKFGEASATVSLLEEADYEAYIKSQQEMMYQTQVTAYVQEPKVAEVAPPISYGDFDKEYEKEQALIRKKMAKDTVMVRTFVEDEEFHISSFEERLIKEIELRIIKTTLDELLEEDGEEMMIDISESEAIGAGFDLRLKNYRTFEGTGVTFHCKTTGYPLPKIAWYKDGKRIRHGERYHMEVLQDGSASLRLPVVLPEDEGIYTVFASNMKGNAICSAKLYVEPVAPTATPGYMPGPEVMRRYRSISPRSPSRSPARSSPSCSPARRLDETDEGQLERLYKPVFVLKPTSVKCSQGQTARFDLKVVGRPMPETYWFHNGQQVVNDYTHKIVIKEDGTQSLIIVPAMPEDSGEWAVIAQNRAGKASVSVTLSVEAKEDLVRPRFVERLRNVSVKEGSRLHMAVKATGNPNPDIVWLKNSDIIVPHKYPRIRIEGTKGAAALNIESTARQDAAWYTATAINKAGRDTTRCKVNVEVEHAEPEPERRLIIPKGTYKAKEIAAPELEPLHLRYGQEQWEEGDLYDKEKQQKPFFKKKLTSLRLKQFGPAHFECRLTPIGDPTMVVEWLHDGKPLEAANRLRMINEFGYCSLDYGVAYSRDSGVITCRATNKYGTDHTSATLIVKDEKSLVEESQLPEGRRGMQRIEELERMAHEGALPAVAVDQKEKQKPELVLVPEPARVLEGETARFRCRVTGYPLPKVNWYLNSQLIRKSKRFRLRYDGIHYLDIVDCKSYDTGEVKVTAENPEGFIEHKVKLEIQQREDFRSVLRRAPEPRHEPVVTEPGKLLFEVQKIDKPAEATTKEVVKLKRAERITHEKLSEESEELRSKFKRRTEEGYYEAITAVELKSRKKDESYEEMLKKTKEELLHWTKEIPEEEKKALPPEGKITIPTFKPEKVELSPSMEAPKIFERIQSQTVAQGTDAHFRVRVVGKPDPECQWFRNGVQIERTDRIYWYWPEDNVCELVIRDVTADDSASIMVKAVNIAGETSSHAFLLVQAKQLISFIQNLQDVVAKERDSMATFECETSEPFIKVKWFKNGIEIHSGEKYRMHSDRKAHFLSVLAVEMSDADDYSCALVEDESVKTTAKLIVEGAVVEFIKELEDVEVPESFTGELECEVSPEDIEGKWYHGDVELSSNHKYVLASRRGRRILTIKDVNKDDQGEYSFVVDGKRTHCKLKMKPRPMTILQGLTDQKVCEGDIVQLEVKVSVENVEGVWMKDGHEIQSSDRIHIVLDKQSHMLLIEDATQEDSGTYSFSIPGLELSTTGQVTVYSVEIIVPLKDVHVVEGTKAILECKVSAPDVTSSKWYLNDHQIKPDERVQAVCKGTKQRLVITRTHASDEGHYKLMVGKVETSCNVTVEEIEIIRGLHDITCTETQNVSFEVELSHSGIDVIWHFKGQEIKAGPKYKIEARGKIYKLTVVKMMKDDEGEYVFYAGGKKTSGKLIVAGGAISKPLADLTVAESQRAVFECEVANPESEGQWLKNGKPLPMTDQYRAETDGVKRRLNIPAAKMDDMGEYSYEIASSKTSAKLHVEAVKIKKTLKNLTVTETQEAVFSVELSHPDVKGALWIKNGVELESNDKYEISVKGTVHTLKIKHCVVTDESVYSFKLGKIGANARLHVETVKIIKKPKDVTALENAVVSFELSVSHDTVPVRWFHKNVELKQSDKYKMISQRKVHKLMLHNISPADAGEYTAFVGQLECKAKLFVETIHITKTMKSIEIPETKTASFQCEVSHFNVPSVWLKNGVEIEMSEKFKIVVQGKLHQLNIMNTSSEDSAEYTFVCGNDRVSATLTVKPILITSMLEDINAEEKDTITFEVTVNYEGISYKWLKNGVEIKSTDKCQIRTKKLTHSLSIRNVHFGDAAEYSFVAGKAASSATLYVEARHIEFRKHIKDIKVVEKKRAIFECEISEPDVQVQWMKDGQELQIGDRMKIQREKYVHRLIIPSTKMSDAGQYTVVAGGNTSSANLIVEGRDVRIRSIRKEIQVIERQRAEIEFEVNEDDIEPQWYKDGIEINFHYEERYSYVVERRIHRMSIFETTYSDAGEYTFVAGRNRSSVVLYVNAPEPPQIIQELQPTTVESGKPARFCAIISGKPQPKVSWYKDDQQLSPGFKCKFLHDAQEYTLLLIETFPEDSAVYTCEAKNDYGVATTSASLSVEIPEVVSPELEVPVYPPAVIVPLRDAVTSEGQSARFQCRVTGTDLKVSWYSKDREIKPSRFFRMTQFEDTYQLEIAEAYPEDEGTYTFVASNSVGQVTSTAILKLEAPEKIMYEKLEEEIEMEVKVAPILRRRLEPLEVAVNHVAKFTCEVETTPNVKFQWYKAGREIYDGDKYSIRSSNYLSTLEIPRPQVVDCGEYSCKASNQHGSVSSTAFLTVTEPPRFIKKLDSSRLVKQHDSTRYECKVGGSPEIKVTWYKGETEIHPSEKYSMSFVDSVAVLEMHNLSVEDSGDYSCEAQNPAGSASTSTSLKVKAPPAFTKKPHPVQTLKGSDVHLECELQGTPPFQISWYKDKREIRSSKKYKVMSENYLASIHILNVDTADVGEYHCKAVNDVGSDSCIGSVTLRAPPTFVKKLSDVTVVVGETIELQAAVEGAQPISVLWLKDKGEIIRESENLWISYSENVASLKIGNAEPTNAGKYICQIKNDAGFQECFAKLTVLEPAVIVEKPGPVKVTAGDSCTLECTVDGTPELTARWFKDGNELSTDHKYKISFFNKVSGLKILNAGLEDSGEYTFEVKNSVGKSSCTASLQVSDRIMPPSFTRKLKETYGQLGSSAVLECKVYGSPPILVSWFHDGQEITSGDKYQATLTDNTCSLKVNGLQESDMGTYSCTATNVAGSDECSAFLSVREPPSFVKKPEPFNVLSGENITFTSIVKGSPPLEVKWFRGSIELAPGHKCNITLQDSVAELELFDVQPLQSGDYTCQVSNEAGKISCTTHLFVKEPAKFVMKVNDLSVEKGKNLILECTYTGTPPISVTWKKNGVILKHSEKCSITTTETSAILEIPNSKLEDQGQYSCHIENDSGQDNCHGAITILEPPYFVTPLEPVQVTVGDSASLQCQVAGTPEMIVSWYKGDTKLRGTATVKMHFKNQVATLVFSQVDSDDSGEYICKVENTVGEATSSSLLTVQERKLPPSFTRKLRDVHETVGLPVTFDCGIAGSEPIEVSWFKDNVRVKEDYNVHTSFIDNVAILQILKTDKSLMGQYTCTASNAIGTASSSGKLVLTEGKTPPFFDTPITPVDGIIGESADFECHISGTQPIRVTWAKDNQ

SEQ ID NO: 44 - protein, 817 aa, Gallus gallus
RESDHRAWTPVSYTVTRQNAVVQGLTEGKAYFFRIAAENIIGMGPFTETTKELIIRDPITVPDRPEDLEVKAVTKNSVTLTWNPPKYNGGSDITMYVLESRLIGKEKFHRVTKEKMLDRKYTVEGLKEGDTYEYRVSACNIVGQGKPSFCTKPITCKDEIAPPTLDLDFRDKLIVRVGEAFSLTGRYSGKPTPKITWLRDDIVLKEDDRTKIKTTPTTLCLGILKSVREDSGKYCVTVENSTGSRKGFCQVTVVDRPSPPVGPVIFDEVHKDHMIVSWKPPLDDGGSEITNYIIEKKDTHRDLWMPVTSATVKTTCKIPKLLEGREYQVRIYAENLYGISDPLISDEMKAKDRFSVPDAPEQPVIKDVTKDSAVVVWNKPYDGGKPITNYIVEKKETMATRWVRVTKDPIFPSTQFKVPDLLEGCQYEFRVSAENEIGVGNPSPPSKPIFARDPIVKPSPPVNPEAVDKTKNSVDLTWQPPRHDGNGKIIGYLVEYQKVGDEEWKKANLTPDSCPETKYKVTGLTEGLTYKFRVMAVNAAGESEPAYVPDPVEVKDRLEPPELILDANMAREQHVRAGDTLRLSAVIKGVPFPKVSWKKEDKEVSPKADIEVTGVGSKLEIRNTVHEDGGIYSLTVENPAGSKTVSVKVLVLDKPGPPRNLQVSDVRSDSCYLTWKEPEDDGGSVITNYVVERKDVASAQWVSVSSSSKKRSHMAKYLMEGTQYLFRVAAENLFGRGPYVETLKPIKAMDPLHPPGPPKNLHHIDVDKTEVSLVWNRPDRDGGAEITGYLVEYQEDGADEWTKFQTVPMLDCVVTGL

SEQ ID NO: 45 - protein, 595 aa, Gallus gallus
VTTKQTVISRTREGIVTSQEQRHISREKVRKEPEKTPVPTVIIVTAKVKEQERISTAREEISARHEQVHVSHEQIRREDVKPSVPKVVITTDKPKAPVLISQSKEGIATKKEHVSISHEKIKKEAKKTTAVLKIIAPVTVSRTREEITAKPEQMHLAYDQIEAGKRAEAVATVVAAVDQARVRSPWETEQVDETYVKKKTLEYGYKEHAVKAHEAQAEHHVATKEVKTVYVPPEKHIPAAEKKEVHVSTEIKRETEAKIEKTNHIEHPRPRTASPHFTVSKIAVPKPDHTYEVSIAGSAMATLEKELSATSAEQKITKPVKPPQLKPHEVKIKPESAPPPFPFTEAAETYKAHYDVETKKEVDVSIKGGAVREDHLLLRKESEAKVTETARVPVPAEIPVTPTHLVWGLKNKTVTEGESVTLECHISGHPQPTVTWYRDDYKIESSMDFQITFKAGLARLVIREAFAEASGRFTCTATNKAGSVSTSCHLHVKVSEETETRETISEKVVTEEKSYVETQDVVMEDASPPPEEVSGEPVPPFFIRKPVVHTLIVGGSIIFEFHVGGNPKPHVLLKKGGVPLTTGYRYKVSYKRETG

SEQ ID NO: 46 - protein, 34340 aa, Bos taurus
MATQAPTFTQPLQSVVVLEGSTATFEAHISGFPVPEVSWFRDGQVISTSTLPGVQISFSDGRAKLTIPAVTKANSGRYSLRATNGSGQATSTAELLVTAETAPPNFTQRLQSMTVRQGSQVRLQVRVTGIPTPVVKFYRDGAEIQSSLDFQISQEGELYSLLIAEAYPEDSGTYSVNATNSVGRATSTAELLVQGEEVVPAKRTKTIVSTAQISETRQTRIEKKIEAHFDARSIAAVEMVIDGAAGQQLPHKTPPRIPPKPKSRSPTPPSIAAKAQLARQQSPSPIRHSPSPVRHVRAPTPSPVRSVSPAGRISTSPIRSVKSPLLVRKAQTPTMPPGPEVPPPWKQEGYVASSEAEMRETTVTSATQIRTEERWEGRYGIQEQVTISGAAGAAASTTFAAGAVAAGAAEVKQEADKSAAVATVVAAVDMARVREPVISAVEQTAQRTTTTAVHIQPAHEQIRKEAEKTAVTKVVVAADKAKEQEVKARTREVITTKQEQVHVTHEQIRKETEKAFVPKVVISAAKAKEKETKITGEITTKQEQKQITQETIIQKAETAAVSMVVVETAKPTTLETILGAQEEIVTQQDQMHITHEKILKETRKTVVPKVIVATPKVKEQDVVSRTREGITTKREQVQVTHEKMRKEAEKTALSTIAVATAKAKEQETILRTREEMATRQEQIQVTHGKVGVGKKAEAVATVVAAVDQARVREPREPRHVEESYAQQTTLEYGYKEHISATKVAEQPPRPASEPQVVPKAVKPGVIHAPSETHIATTDQMGMHISSQIKKTTDLTSERLVHVDKRPRTASPHFTVSKISVPKTEHGYEASIAGSAIATLQKELSATPSAQKVTKSVKAPTVKPAEARVRAEPTPSPQFPFAETPETFKSQAGIEVKKEVGVSITGVAVREERFEALRERETKVTETARVPAPVEIPVTPPTLVSGLKNVTVIEGESVTLECHISGYPSPKVTWYREDYQIESSIDFQITFQSGIARLLIREAFAEDSGRFTCTAVNEAGTVSTSCYLAVQVSEEFEKETTAVAEKITTEEKRFVESRDVVMTDTSITEEHAGPGEPAAPFFITKPVVQKLVEGGSIVFECQVGGNPKPHVYWKKSGVPLTSGYRYKVSYNKQTGECRLVISMTFADDAGEYTIVIRNKHGETSASASLLEEADYESLMKSQQEMLYQTQVTAFVQEPKVGEIAPGYVYSEYEKEYEKEQALIRKKMAKDTVMVRTFVEDQEFHISSFEERLIKEIEYRIIKTTLEELLEEDGEEKMAVDISESEAIEAGFDLRVKNYRILEGMGVTFHCKMSGYPLPKIAWYKDGKRIKHGERYHMDFLQDGRASLRIPVVLPEDEGIYTAFASNMKGNAICSGKLYVEPAAPLSAPTYIPTPEPVSRIRSISPRSVSRSPIRMSPARMSPARMSPGRRLEETDESQLERLYKPVFVLKPTSFKCVEGQTARFDLKVVGRPMPETFWFHDGQQIVNDYTHKVVVKEDGTQSLLIVPATPSDSGEWTVVAQNRAGKSSISVILTVEAVEHQVKPVFVEKLRNLNIKEGSRLEMKVRATGNPNPDIVWLKNSEIIVPHKYPKIRIEGTKGEAALKIDSTVSQDSAWYTATAINKAGRDTTRCKVNVEVEFAEPEPERRLIIPRGTYRAKEIAAPELEPLHLRYGQEQWEEGDLYDKEKQQKPFFKKKLTSLRLKRFGPAHFECRLTPIGDPTMVVEWLHDGKPLEAANRLRMINEFGYCSLDYAVAYSRDSGVITCRATNKYGTDHTSATLIVKDEKSLVEESQLPEGRKGLQRIEELERMAHEGALTGVTTDQKEKQKPDIVLFPEPVRVLEGETARFRCRVTGYPQPKVNWYLNGQLIRKSKRFRVRYDGIHYLDIVDCKSYDTGEVKVTAENPEGVIDHKVKLEIQQREDFRSVLRRAPEPKPEFHVHEPGKLQFEVQKVDRPVDTTETKEIVRLKRAERITHEKVSEESEELRSKFKRRTEEGYYEAITAVELKSRKKDESYEELLRKTKEELLHWTKELTEEEKKALAEEGKITIPTFKPDKIELSPSMEAPKIFERIQSQTVGQGSDAHFRVRVVGKPDPECEWYKNGVKIERSDRIYWYWPEDNVCELVIRDVTAEDSASIMVKAINIAGETSSHAFLLVQAKQLITFTQELQDVVAKEKDTMATFECETSEPFVKVKWYKDGVEIHTGDKYRMHSDRKVHFLSVLMIDTSDAEDYSCVLVEDENVKTTAKLIVEGAVVEFVKELQDIEVPESFSGELECIITPENIEGKWYHKGVELKSNGKYTITSRRGRQNLTVKDVTKEDQGEYSFVVDGKKTTCKLKMKPRPIAILQGLSDQKVCEGDIVQLEVKVSLENVEGVWMKDGEEVQPGDRVHIVIDKQSHMLLIEDMTKEDAGHYSFTIPSLGLSTHGRVSVYSVDVITPLKDVNVLEGTKAVLECKVSVPDVTSVKWYLNDEQIKPDDRVHAIVKGTKQRLVINRTHASDEGPYKLVVGRVETSCDLSVEKIKIIRGLRDLTCTETQNVVLEVELSHSGIDVLWNFKDKEIKPSSKYKIEAHGKIYKLTVLNMMKDDEGEYTFYAGENRTSGKLTVAGGAISKPLTDQTVAESQEAVFECEVANPDSEGEWLKDGKHLPLSKTIRSESDGRKRRLIIAATKLDDIGEYTYKVATSKTSAKLKVEAVKIKKTLKNLTVTETQDAVFTVELTHPNVKGVQWIRNGVVLESSDKYTVSVKGTVYSLRIKNCAVADESVYGFKLGKLGASARLHVETVKIIKKPKDVTALENATVSFEVSVSHDTVPVKWFHKNVEIKPSDKHRLVSERKVHKLMLQHISPSDAGEYTAVVGQLECKAKLFVETLHITKTMKNIEVPETKTASFECEVSHFNVPSMWLKNGVEIEMSEKFKIVVQGKLHQLIIMNTSTEDSAEYTFVCGNDQVSATLKVTPIMITSMLKDINAEEKDTITFEVTVNYEGISYKWLKNGVEIKSTDRCQMRTKKLTHSLNIRNVHFGDAAEYTFVAGKATSTATLYVEARHIEFRKHIKDIKVLEKKRAMFECEVSEPDITVQWMKDGQELQLVDRIKIQKEKYVHRLLIPSTRMSDAGKYTVVAGGNMSSANLFVEGRDVRIRSIKREVQVIEKQRAVVEFEVNEDDVDAHWYKDGIEINFQVQERHQYVVERRIHRMFISETRHSDAGEYTFVAGRNRSSVTLYVNAPEPPQILQELQPITVQSGKPARFCAVISGRPQPKISWYKEEQLLSTGFKCKFLHDGQEYTLLLIEAFPEDAAVYTCEARNDYGVATTSASLSVEVPEVVSPDQEMPVYPPAIVSPLQDAVTSEGRPAHFQCRVSGTDLKVSWYSKDKKIKPSRFFKMTQFEDTYQLEVAEAFPEDEGIYTFVASNAVGQVSSTATLRLEAPEAMLHEQIEMEMKEFSISLLSGKEERPQTSSWDLQLFDINETLEPLSEPSVYSPKFDSKKEGNAPVFIKEVSNAEISIGDVAKLFVTVTGIPTPQIQWFFNGAMLTPSADYKFVFDGNDHSLIILFTKLEDEGEYTCIASNEYGQAMCSAYLKINSEAEGHEEPESAEKSLEKLQGPCPPYFLKELKPVHCVQGLPAIFEYVVLGEPAPTVLWFKENKQLCTNVYYTIIHNPDGSGTFIVNDPQKEDGGLYVCKAENVWGGSTCAAELFVLLEDTDMTDATCKAAPTPEAPEGFPHTSVKDPAVETFDSEQEVVTFVKDAILKASLIAEEKQQFSYEHVVKTNELSSQVTLKAEQLQSTVILEHDMPTPESTRELLSISGTIHVQPIKELPPSLQLQIAQSQDTLPREDTLMCQEPESQVVLSDTEKIFPSFMSIEEISLSTVESLQTLLAEPEESYPQPLIKETPAHSYPTSVAEEVLLSKDKTVSAVDRGQRETSQKQESQNALFLNQNLAEGHAQSLQGPDVTVSRVSSEHTCTESEILMESVDQLDSAGQDFAARIEEGQSLRFPLACEEKQVLLKEEHSDTLALPLNQTTEYKKEPMAVNGVQEVQGSDFLSKESLLPGIPEEQRLNLKTQIRSALQAAVASEQLSLFSEWLRNIEQVEVKAIDFTQEPNCIMCTYLITSVKSLTEEVTVTIKGIDPQMADLKTELKEALCSICEEMNILTAEEPGIEKGATVVLQEDVSFSSSQKLEAITEPEAESKYLVSKEEISYFKVESQVKAVGTTTASAAVSDEKQDELPKPSEEKEKVSEGGTEEVGTVEIQEAEDEEDRKLGEDGPDVQTPLVDTIAEEGDIISLTSCITNAKEVNWYFESKLVPSDEKFKCLQDQNTYTLVIDKVNREEHQGEYTCEALNDNGKATTSAKLTVVKRAAPVIRRRIEPLEVALGHLAKFTCEIHSAPNVRFQWFKAGREIYESDKCSIRSANYVTTLEILRTQVVDCGEYTCKASNEYGSVSCTATLTVTEAYPPTFFSRPKSVTTFVGKAAKFLCTVTGTPVIETIWQKDGMALSPSPTCKISDVDNKHILELSNLTVHDKGVYSCKASNKFGADTCQAELDIIDKPHFIKELEPVQSAINKKIHLEGQVDEDRKVTITWSKNGQKLPPGKDYKIYFEDKIASLEIPLAKLKDSGTYVCTASNEAGSSSTSATVTIREPPSFVKKVDPSYLMLPGESARLHCRLKGSPVIQVTWFKNNKELTESNTIRMSFVNSEAVLDITDVKVEDSGTYSCEAVNDVGSDSCSAEIVVKEPPSFIKTLEPADIVRGTNALLQCEIAGTGPFEISWFKDKKQIRSSKKYRLFSQKSTVSLEIFSFNSADVGDYECVVANEVGKCGCVATHLLKVEPPTFVKKVDDFTALAGQTVTLQAAVRGSEPISVTWMKGQEIIKEDGKIKMSFANGVAVLIIPDVQISFGDKYTCLAENEAGSQTSVGELIVKEPAKIIERAELIQVTAGDPATLEYTVAGTPELKPKWYKDGRPLVASKKYRISFKNNVAQLKFYSAELHDSGQYTFEISNEVGSSSCETTFTVLDRDIAPFFTKPLRNVDSVVSGTCRLDCKIAGSLPMRVSWFKDGKEVTSSDRYRIAFVEGTASLEISRVDMSDAGNFTCRATNSVGSKDCSGALIVQEPPSFVTKPASKDILPGSAVFLKSTFQGSTPFTIRWFKGDKELVSGGNCYINKEALESSLELYSVKTTDSGTYTCKVSNVAGAVECSANLFVKEPATFVERLELSQLLKKGDTTQLACKVTGTPPIKITWFANDREIKESSKHKMSFVESTAVLRLTDIAVEDSGEYMCEAQNEAGSDHCSSILIVKESPYFTKEFKPIEVLKEYDVMLLAEVAGTPPFEITWFKDNTTLRSGRKYKTFIQDRLVSLQILKFVAADVGKYQCRVTNEVGSSTCSARVTLREPPTFVKKIESTSSLRGGTAAFQATLKGSLPITVTWLKDNEEITEDDNIRMTFENNVASLYLSGIEVKHDGKYVCQAKNDAGIQRCFAVLSVKEPATIIEEAVSIDVTQGDPATLQVKFSGTKEITAKWFKDGQELTLGQKYKISVTDTVSILKIISTEKRDSGEYTFEVQNDVGRSSCKASINVLDLIIPPSFTKKLKKMDSIKGSSIDLECIVAGSHPISIQWFKDDQEITASEKYKFSFHDNTAFLEISQLEGSDSGTYTCSATNKAGHNQCSGHLTVKEPPYFLEKPQSQDVNPNTRVQLKALVGGTAPMTIKWFKDNKELHSGAARSVWKDDTSTILELFSAKAADSGTYICQLSNDVGTATTKASLFVKEPPQFIKKPSPVLVLRNGQSTTFECQITGTPEIRVSWYLDGNEITAVEKHGISFIDGLATFQISGARVENSGTYVCEAQNDAGTASCSIELKVKEPPTFIRELKPVEVVKDSDVELECEVMGTSPFQVTWLRNNKEIRSSKKYTLTDRVSVFNLNINRCDPSDTGDYQCIVSNEGGSCSCSARVSLKEPPSFIKKIENITTVLKSSATFQSTVAGSPPISITWLKDDQILDEDDNVHISFVNNVATLQIRSVDNGHSGRYTCQAKNESGVERCYAFLLVQEPAQIIEKAKSVDVTERDPVTLECVVAGTPELKVKWLKDGKQIVPSRYFSMSFENNVASFRIQSVMKQDSGEYTFKVENDFGSSSCDAYLRVLDQNIPPSFTKKLTKMDKVLGSSIHMECKVSGSLPISAQWFKDGKEISTSAKYRLVCHENTVSLDVNNLELEDTANYTCRVSNVAGDAACSGILTVKEPPSFLVKPERQQAIPDSTVEFKAVLKGTPPFKIKWFKDDVELASGPKCFIGLEGSTSFLNLYSVDASKTGQYTCQVTNDVGSDSCTTTLLVTEPPKFVKKLEATKIVKAGDSARLECKITGSPDIRVVWYRNEHELPASDKYRMAFIDSVAVLQMNYLGTEDTGDFICEAQNPAGSTSCSTKVIVKEPPVFSSFPPVVETLKNAEVSIECELSGTPPFEVVWYKDKRQLRSSKKYKIASKNFHASIHILNVDTLDIGEYHCKAQNEVGSDTCICIVKLKEPPRFVSKLNSLTVVAGEPAELQASIEGTQPISVQWLKEKEEVVRESENIRITFVENVATLQFSKAEPANAGKYICQIKNDGGMRENMATLSFISEPAVIVEKAGSMTVTVGETCTLECKVAGTPELSVEWYKDGKLLTSSQKHKFSFYNKISSLKILSVEKQDAGTYTFQVQNNVGKSSCTAVVDVSDRMVPPSFTRRLKDTIGVLGTSCILECKVAGSSPISVAWFHGKTKIVSGAKYQTTFSDNVCTLQLNSLDSSDMGSYTCVAANVAGSDECRAVLAVQEPPSFVKEPEPLEVLPGKNITFTSVIRGTPPFKVGWFRGARELVKGDRCNIYFEDTVAELELFNVDISQSGEYTCVVSNNAGQTSCTTRLFVKEPAVFVKKLSDHSVEPGKSIILESTYKGTLPISVTWKKDGFNITPSEKCSIVTTEKTCILEILNSTKRDAGRYSCEIENEAGRDVCEALVSTLEPPYFVTELEPLEASVGDSVSLQCQVAGSPEITVSWYKGDTKLRPTPEYRTYFTNNVATLVFNKVNINDSGEYTCKAENSIGTASSKTVFRIQERQLPPSFARQLKDIEQTVGLPVTLTCRLNGSAPIQVSWYRDGVLLRDDENLQTSFVDNVATLKILQTDLSHSGQYSCSASNPLGTASSSARLTAREPKKSPFFDIKPVSIDIIAGESADFECHVTGAQPMRITWSKDNKEIRPGGNYTITCVGNTPHLRILKVGKGDSGQYTCQATNDVGKDMCSAQLSVKEPPKFVKKLEASKVAKQGESIHLECKISGSPEIKVSWFRNDSELHESWKYNMSFVDSVAVLTINEASAEDSGDYICEAHNGVGDASCSTALTIKAPPVFTQKPSPIGALKGSDVVFQCEISGTPPFEVVWVKDRKQVRSSKKFKITSKNFDASLHILNLEAADVGEYYCKATNEVGSDTCVCTLKFKVEPPRFVKKLSDTSTLVGDAVELRAVVEGFQPISVVWLKDKGDVIRESENTRISFVDNVATLQLGSPEASDSGKYVCQIKNDAGMRECSAILTVLEPARIIEKPEPMTVTTGNAFALECVVTGTPELSAKWFKDGREIAADSKHHITFVNKVASLKIPCAEMSDKGLYSFEVKNSVGKSTCTVSVHVSDRIVAPSFIRKLKDINAIVGASVVLECRVSGSAPISVGWFQDGNEIVSGPKCQSSFSENVCTLNLSFLEPSDTGTYTCVAANVAGSDECSAVLTIQEPPSFEQTPDSVEVLPGTSLTFTSVIRGTPPFKVKWFKGSRELVSGESCSISLEDFVTELELFEVEPLQSGDYSCLVTNDAGSASCTAHLFVKEPATFVKRLADFSVETGSPIVLEATFTGTPPISVSWMKNEFALTQSQNCSITMTEKSTILEILDSTIEDYAQYSCLIENEAGQDICDAVVSVLEPPYFIEPLEHVEAVIGEPITLQCKVDGTPEIRISWYKEHTKLRSAPAYKMQFKNNVASLVINKVDHSDVGEYTCKAENIVGAVASSSVLVIKERKLPPSFARKLKDVQETLGFPVAFECRINGSEPLQVSWYKDGVLLKDDANLQTSFVHNVATLQILQTDQSHVGQYNCSASNPLGTASSSAKLVLLEHEVPPFFDLKPVSVDLALGESGSFKCHVTGTAPMKITWAKDNREIRPGGNYKMTLVENTATLTVLKIGKGDAGQYTCYASNVAGKDSCSAHLGVQASIEPPRFIKKLEPSRIVKQDESTRYECKIGGSPEIKVLWYKGETEIQESSKFRMSFVDSVAVLEMHGLSVEDSGDYTCEACNAAGSASSTTSLKVKALEPPIFRKKPHPVETLKGADVHLECELQGTPPFQVSWHKDKRELRSGKKYKIMSENFLTSIHILSVDAADVGEYQCKATNDVGSDTCVGSITLKAPPRFVKKPSDVSTIVGEEVQLQATVEGAEPISVVWFKDKGEIVRESDNIWISYSENTATLQFSRAETANAGKYTCQIKNDAGMQECSATVSILEPAAIVEKPESITVTTGDTCTLECSVTGTPELTTKWFKDGKELTSDNKYKISFFNKVSGLKIINVAPSDSGVYSFEVQNPVGKDSCTASVQVSDSDRIVPPSFTRRLKETNGLSGSSVVMECKVYGSPPISVSWFHERNEISSGRKYQTTLTDNTCALTVNMLEESDAGNYTCIATNVAGSDECSAPLTVREPPSFVQKPDPMDVLTGTNVTFTSIVKGTPPFSVSWFKGSTELVPGDTCNVSLEDSVAELELFDVDTSQSGEYTCIVTNEAGKVSCTTHLYVKAPARFVKKLNDYSIEKGKPLILEGTYTGTPPISVTWKKNGINVTPSQRCNITTTERSAILEIPSSTVEDAGQYNCYIENTSGKDSCSAQILILEPPYFVRQLEPVKVTVGDSASLQCQLGGTPEIAVSWFKGDTKLRPTATYKMHFRNNIATLVFNQVDSNDSGEYICRAENSVGEVSSSTFLTVQEQKLPPSFSRQLRDVQETVGLPVVFECAVSGSEPISVSWFKDGRPVKDSPNIQASFLDNVATLNIFQTDRSFAGQYSCTATNPIGSASSSARLILTEGKNPPFFDIPLAPVDAVVGESADFECHVTGTQPIKVAWAKDNREIRSGGNYQISYLENSAHLTILKVDKGDSGQYTCYAVNEVGKDSCTAQLNIKERLIPPSFTKKLSETVEETEGNSFKLEGRVAGSQPISVAWYKNNIEIHPTSNCEITFKNNTLLLQIKRAGMDDAGLYTCKVSNDAGSALCTSSIVIKEPKKPPVFDQHLTPVTASEGEFVQLSCHVWGSEPIRIQWLKAGREIKPSDRCSFSFANGTAVLELKDVTKADAGDYVCKASNVAGSDTSKSKVTIKDKPAAVPAAKKAAVDGKLFFVSEPQSIRVVEKTTATFIAKVGGDPIPNVKWTKGKWRQLNQGGRILIHQKGDEAKLEIRDTTKTDSGLYRCVAFNKHGEIESNVNLQVDERKKQEKIEGDLRAMLKKTPVLKKGAGEEEEIDIMELLKNVDPKEYEKYARMYGITDFRGLLQAFELLKQTEGEETHRLEIEEIEKSEKDEKEFEELVSFIQQRLSQTEPVTLIKDIENQTVLKDNDAVFEIDIKINYPEIKLSWYKGTEKLEPSDKFEISIDGDRHTLRVKNCQLKDQGNYRLVCGPHIASAKLTVIATEPAWERHLQDVTLKEGQTCTMTCQFSVPNVRSEWFRNGRILKPQGRYKTEVEHKVHKLTIADVRAEDQGRYTCKHEDLETSAELRIEAEPIQFTKRIQNIVVSEHQSATFECEVSFDDAIVTWYKGPTELTESQKYNFRNDGRCHYMTIHNVTPDDEGVYSVIARLEPRGEARSTAELYLTTKEIKLEMKPPDIPDSRVPIPTMPIRAIPPEEIPPTAVPPIPLLLPLPEEKKPPEKRVEVTKKAVKKDAKKVVAKPKEEAPPPKVIEVPKKPPRPTALIPAEAPEIIDVSSKAEEVKITTITRKKEVQKEKEAVYERKQAVYEEKKVYIESLEEPYDELEVETYTEPYEEPYYEEPEEDYEETQVEAKREVHEEWEEDFEEGQEYYEREEGYDEGEEEWEETYQEREVVQVQKEVYEESRERKIPAKVPEKKELPPPKVVKKPVVEKIEKTSRRMEEEKVQVTKVPEVSKKIVPQKPSRTPVQEEIIEVKVPAVHTKKMVISEEKMFFAAHTEEEEEVSVTVPEVQKKTVTEEKVHVAVSKKIEPPPKVPEPPKKPEEVVPVPVPKKVEPPPAKVPEVPKKPVPEEKKPVPVPKKEPAAPPKVPEVPKKPVPEEKVPVPIAKKKEAPPAKVPEVQKRVVTEEKITIVTEREESPPPAVPEIPKKKLPEEKRPVPRKEEEVPPPKVPAVPKKPVPEEKVPVPVPVAKKAPPPRAEVSKKMVMEEKRFAAEEKLSVTVPQRVEVMRHEVSAEKEWRYSEEEERVSVSVYREEEREEEEVEVTEYEVMEEPEEYIVEEEEHFISEEVEAEPAEESFQVPEKKIIPKPKIPAKVEEPPPAKVPEAPKKIVPEKKIPAAVPKKEKVPPTKVPEEPKKPVPEKRAPPKVAKIEEPPPTKVTERHMQITQEEKVHVAVTKKVEPPRPKVPEEPKRAVPEEKFPKLKPRKEEEPPAKVTELRKRAVKEEKVSIEVAKREPPAAKEVTVTAEKEWAYTKEEEAVSVEREEEYEEYEEYDYKEFEEYEPTEEYDQYEEYEEREFEHYEEYKEHEEYVTEPEKPVPVKPIQEEPVPTKPKAPPAKVPKKVIPEEKVPVPIPKKLKPPPPKAREEAKKVVEEKIQISVIKREKEQVTEPAAKVPMKPKRVVTEEKVPVPKKEVAAPVRVPEVPKKEEPEEIAFEEEVVTHEEEYYEEEEEEEEYIHEEEEIITEEEVVPVKVFKVPEVPKKPVPEEKKPVPVPKKKEAPPAKVPEIPKKPEEKVPVPVPKKEKAPPAKVPEVPKKPVPEEKVPVPEEKVPVPVPKKVEAPPAKVPEVPKKPVPEKKVPVPAPKKVEAPPAKVPEVPKKVLPEEKKPTPIRKKVEPPPPKVPKKREPVPVPIPAALLREEKVVHKEEIVVEEEEVLPEEEEISPEEEVPLEEEEEEEEVLPEEEEVPPEEEEVPPEEEEVPPEEEEFVPEEEVVPEKEEVLPEIKPKVPVPARVPEVKKKVPEKKVIIPKKEEVPPAKVPEVPKKVEEKRIIVPEEEEVPPVEVFEEPEEPIPEEEIPEEAPIVEEVEEEAPPRVPEVVKKAVPEAPTPVPKKVEVPPAKVPKKVPEEKPPVPVQKKEAPPAKVPEVPKKVPEKKVPVPKKEVVPPAKGRAVLEEKVSVAFHKEEVVEERTELEIVEAQEEALEEEFHEVEEYFEEEEFHEVEEFLKVEKYRAEEEVHRAEEVHRVIEVLEAEEEEAYEKPKAPPKGPEISEKVIPPKKPPTKVVPRKEPPAKVPEVPKKIVVEEKVHVPEEPKVAPAKVPEAPKEVVPEKKVPAAPPKKPEVPPVTVPEAPKEVVPEKKVPVVPPKKPEVPPAKVPEVPKAAVPEKKVPEAIPPKPESPPPAVPEAPKEVVPEKKVPVMPPKKPEVPPAKVPEAPKEVVPEKKVPVMPPKKPEVPPAKVPEVPKAAVPEKKVPEAIPPKPESPPPAVYEEPEEIAPEEPPVEVVEEPEPVPAPPPKVTVPPKKPVPEKKPPAVVAKKPEPPPAKVPEVPKEVVPEKKVPPVVPKKPEVPPPKVPEVPKEVVPEKKVAVPKKPEVPPAKVPEVPKKPVLEEKPAIPIPEKVESPPPEEEEEPVPVVEEEEPVPVVEEEEPVPVVEEVEPEAPPPAVPEEPKKIVPEKKVPVIKKPEPPPPKEPEPEKKVIEKPKLKPRPPAPPPAPPKEDVKEKIFQLKAVSKKKVPEKPAVPEKVELTPLKVAGGEKKVRKLLPEPKPQPKEEVILKSVLRKRPEEEEPKVEPKKVEKIKKPAVPEPPPKAVEEVEAPPAVTKKERKIPEPTKVPEIKPPIPLPAPEPKPKPEAEVKIIKPPPVEPPPTPIAAPVTVPVVGKKAEAKAPKEEAAKPKGPIKGVAKKTPSPIEAERKKLRPGSGGEKPPDEAPFTYQLKAVPLKFVKEIKDIVLTEAESVGSSAIFECLVSPSTAVTSWMKDGSNIRESPKHRFIADGKDRKLHIIDVQLSDAGEYTCVLRLGNKEKTSTAKLIVEELPVRFVKTLEEEVTVVKGQPLYLSCELNKERDVVWRKDGKIVVEKPGKIVPGVIGLLRALTINDADDSDAGTYTVTVENANNLECSSCVKVVEVIRDWLVKPIRDQHVKPKGTAVFTCVIAKDTPNIKWYKGYDEIPWEPTDKTEIIRDGNHIHLKVKNAMPEDIDEYAVEIEGKRYPAKLTLGEREVELLKPIEDVTIYEKESASFDAEISEVDVPGQWKLKGELLRPSPTCEIKAEDGKRFLTLHNVKLDQAGEVLYQALNAVTTAILTVKEIELDFAVPLKDVTVPEKRQARFECVLTREANVIWSKGPDIIKSSDKFDIITDGKKHILVINNSQFDDEGVYTAEVEGKKTSARLFVTGIRLKFISPLEDQTVKEGETATFVCELSHEKMHVVWFKNDAKLHTSRTVLISSEGKTHKLEMREVTMDDISQIKAQVKDLSSTANLKVIEADPYFTVKLHDKTGVEKDEIVLRCEVSKDVPVKWFKDGEELLPSPKHSIKADGLRRILKIKKADLKDKGEYVCDCGTDTTKANVTVEARLIKVEKPLYGVEVFVGETARFEIELSEPDVHGQWKLKGEPLTASPDCEIIEDGKKHILVLYNCRLDMTGKVSFQAANAKSAANLKVKELPLIFITPLSDVKVFEKDEAKFECEVSREPKTYRWLKGTQEITGDDRIELIKDGTKHSLVIKSAAFEDEAKYIFEAEDQHTSGKLIIEGIRLKFLTPLKDVTAKERESAVFTVELSHDNIRVRWFKNDQRLHTSKFVSMDDEGKTHSITFRNLSIDDTSQIKVEAMGLSSEAKLTVLEGDPYFTGKLQDYTGVEKEEVILQCEISKADAPVKWFKDGQEIKPSKNAVIKADGKKRMLILKKPLKSDIGQYTCDCGTDKTSGKLNIEDREIKLVRPLYSVEVMETETARFETEISEDDIHANWKLKGEALLQTPECEIKEEGKTHFLILHNCRLDQTGGVDFQAANVKSSAHLRVKPRVIGLLRPLKDITVTAGETATFDCELSYEDIPVEWYLRGKKLEPSDKVVMRSEGRVHTLTLRDVKLEDAGEVQLVAKDFKTQAKLFVKEPPVEFTKPLEDQTVEEEATAVLECEVSRENAKVKWFKNGTEILKSKKYEIVADGRVRKLIIHGCTPEDIKTYTCDAKDFKTSCNLNVVPPHVEFLRPLTDLRVKEKEMARFECEISRENAKVHWFKDGAEIKKGKKYDIISKGAVRILVINKCLLDDEAEYSCEVRTARTSGMLTVLEEEAVFTKNLANIEVSETDTVKLVCEVSKPGAEVTWYKGDEEIIETGRYEILTDGRKRILIIQNAHLEDAGNYNCRLPSSRTDGKVKVHELAAEFISKPQNLEILEGEKAEFVCSISKENFEVQWKRDDKTLESGDKYDVIADGKKRVLVVKDATLQDMGTYVVMVGAARAAAHLTVIEKLRIIVPLKDTRVKEQQEVVFNCEVNTEGAKAKWFRNDEAIFDSSKYIILQKDLVYTLRIRNAQLDDQANYNVSLTNHRGENVKSAANLIVEEEDLRIIEPLKDIETMEKKSVTFWCKVNRLNVTLKWTKNGEEVAFDNRVLYRIDKYKHSLIIKDCGFPDEGEYVVTAGQDKSVAELLIIEAPTEFVEHLEDQTVTEFDDAVFSCQLSREKANVKWYRNGREIKEGKKYKFEKDGSIHRLIIKDCRLDDECEYACGVDDRKSRARLFVEEIPVEIIRPPQDILEAPGADVVFLAELNKDKVEVQWLKNNMIIVQGDKHQMMSEGKIHRLQICDIKPRDQGEYRFIAKDKEARAKLELAAAPKIKTADQDLVVDVGQPLTMVVPYDAFPKAEAEWFKENEPLSSKTVDTTAEQTSFRILKAKKEDKGRYKVVLQNKHGKAEGFINLKVIDVPGPVRNLEVTETFDGEVSLAWEEPLTDGGSKIIGYVVERRDIKRKTWVLATDRADSCEFTVTGLQKGGVEYLFRVSARNRVGTGEPVETDSPVEARSKYDVPGPPLNVTVTDVNRFGVSLTWEPPEYDGGAEITNYVIELRDKTSIRWETAMTVRAEDLSATVTDVVEGQEYSFRVRAQNRIGVGKPSAATPFIKVADPIERPSPPVNLNASDQTQSSVQLTWEPPLKDGGSPILGYIIERCEEGKDNWIRCNKKLVPELTYKVTGLQKGNKYLYRVSAENEAGVSDPSEILGPLTADDADAEPTMDLSAFKDGLEVIVPNPIKILVPSTGYPRPTATWSFGDKVLEAGDRVKMKTLSAYAELVISPSERPDKGIYTLKLENRAKAISGEIDVNVIARPSAPRELKFGDITKDSVHLTWEPPEDDGGSPLTGYVIEKREVSRKTWTKVIDSVTDLEFTVPDLLQGKEYLFKVCACNKCGPGEPAYVDEPVNMSAPATVPDPPENVKWRDRTAKSIFLTWDPPKHDGGSRIKGYIVEKCPRGSDRWVACGEPVADTKMEVTGLEEGQWYAYRVKALNRLGASKPSKPTEEIQAIDTQEAPEIFLDVKLLAGITVKAGTKIELPATVTGKPEPKITWTKADMLLKQDKRITIENVPKKSTVTIMDSKRSDTGRYIIEAVNVCGRATAVVEVNVLDKPGPPAAFDITDVTNESCLLTWNPPRDDGGSKITNYVVERRATDSDMWHKLSSTVKDTKFKATKLTPNKEYIFRVAAENMYGVGEPVQATPITAKFQFDPPGPPTRLEPSDITKDAVTLTWCEPDDDGGSPITGYWVERLDPESDKWVRCNKMPIKDTTYRVKGLTNKKKYRFRVLAENLAGPGKPSKSTEPILIKDAIDPPWPPGKPVVKDIGRTSLMLNWTKPEHDGGAKIDSYVIEMLKTGTEDWVRVAEGVPTTQHLLPGLMEGQEYSFRVRAVNKAGESEPSEPSDPVLCREKLYPPSPPRWLEVINITKNTADLKWTVPEKDGGSPITNYIVEKRDVRRKGWQTVDTTVKDTKCTVTPLTEGSLYVFRVAAENAIGQSDYCEVEDSVLAKDTFTTPGPPYALAVVDVTKRHVDLKWEPPKNDGGRPIQRYVIEKKEKLGTRWVKAGKTSGPDCHFRVTDVIEGTEVQFQVRAENEAGVGHPSEPTEILKIEDPTSPPSPPLDLHVTDAGRKHIAIAWKPPEKNGGSPIIGYHVEMCPVGTEKWMRVNSRPIKDLKFKVEEGVVPDKEYVLRVRAVNAVGVSEPSEISENVVAKDPDCRPTIDLETHDIVVIEGEKLSIPVPFRAVPVPTVSWHKDGKEVKASDRLTMKNDHISAHLEVPKSVRADAGIYTVTLENKLGSATASINVKVIGLPGPCRDLKASDVTKSSCKLTWEPPEYDGGSPILHYVLERREAGRRTYIPVMSGENKLSWTVKDLIPNGEYFFRVKAVNKIGGGEYIELKNPVIAQDPKQPPDPPVDVEVHNPTAEAMTITWKPPLYDGGSKIMGYIIEKIAKGEERWKRCNEHLVPVLTYTAKGLEEGKEYQFRVRAENAAGISEPSRATPPTKAVDPIDAPKVIMKTSLEVRRGDEIALDAIISGSPYPTITWLKDESIITPEEIKKRVEPLVRRRRGEVQEEQPFVLPLTQRLSIDNSQKGESQLRVRDSVRPDHGLFMIKAENDHGIAKAPCTVCVLDIPGPPINFVFEDMRKTSVLCKWEPPLDDGGSEILNYTLEKKDKTKPDSEWMVVTSTLRHCKYSVTKLIEGKEYLFRVRAENRFGPGPPCVSKPFMAKDPFEPPDAPDKPIVEDVTSNSMLVTWNEPKDNGSPILGYWLEKREVNSTHWSRVNRNLLNSLKTKVDGLLEGLTYVFRVCAENAAGPGKFSPPSDPKTAQDPISPPGPPVPRVTDTSSTTIELAWEPPAFNGGGEIVGYYVYKQLVGTNEWSRCTEKMIKPREYTVREIREGADYKLRVTAVNVAGEGPPGETEPVTVAEPQAEPPTVELDVSVKGGIQIMAGKTLRIPAVVTGRPVPTKVWTIEEGELDKDRVEIENVGTKSELIIKNALRKDHGRYVITATNSCGSKFAAARVEVFDVPGPVLDLKPVVTNRKMCLLNWSDPEDDGGSEITGFIIERKDAKMHTWRQPIETERSKCDITGLLEGQEYMFRVIAKNKFGCGPPVEIGPILAVDPLGPPTSPERLTYTERTKSTITLDWKEPRSNGGCPIQGYIIEKRRHDKPDFERVNKHLCPTTSFLVEDLDEHQMYEFRVKAVNEIGESEPSLPLNVVIQDDEVPPTIKLRLSVRGDTIKVKAGEPVNIPADVTGLPMPKIEWSKNETVIEKPTDALKITKEEVSRSEAKTELSIPKATREDKGTYTVTASNRLGSVFRNVHVEVYDRPSPPRNLAVTDIKAESCYLTWDAPLDNGGSEITHYIIDKRDASRKRAEWEEVTNSAVERRYGIWKLIPNGQYEFRVRAVNKYGISDECKSDKVVIQDPYRTPGPPGKPKVLERTKGSMLVSWTPPLDNGGSPITGYWLEKREEGGAYWSRVSRAPITKVGLKGVEFNVPRLIEGVKYQFRAMAINAAGVGPPSEPSDPEVAGDPIYPPGAPSRPEVKDKTKSSITLAWKPPAKDGGSPIKGYIVEMQEEGTTDWKSVNEPDKLLPTCECVVPNLKELKKYRFRVKAVNEAGESEPSDTTGEIPATDIQEVPEVFIDIGAQDCLICQAGTQIRIPAVIKGRPTPKSSWEFDGKAKKAMKDGVHDIPEDAQLETAENSSVIIIPECKRSHSGKYSITAKNKAGQKTANCRVKVMDVPGPPKDLKVSDITRGSCRLSWKMPDDDGGDRIKGYVIEKRTIDGKAWTKVNPNCVSTSFVVPDLIAGQEYFFRVRAENRFGVGAPAETIQRTTARDPIYPPDPPIKLKIGLVTKNTVHLSWKPPKNDGGSPVTHYIVECLAWDPTGTKKEAWRQCNKRDVEELEFTVEDLIEGGEYEFRVKAVNAAGVSKPSATVGPVIVKDQTCPPAIELKEFMEVEEGTDVNIVAKIKGVPFPTLTWFKAPPKKPDNKEPIVYDTHVNKLVVDDTCTLVIPQSRRSDTALYTITAVNNLGTASKEMRLNVLGRPGPPVGPIKFESISADQMTLSWLPPIDDGGSKITNYVIEKREANRKTWVRVSSEPKECTYTIPKLLEGHEYVFRIMAQNKYGIGEPLDSEPETARNLFSVPGAPDKPTVSSVTRNSMTVNWEEPEYDGGSPVTGYWLEMKDTTTKRWKRVNRDPIKAMTLGVSYKVTGLIEGSDYQFRVYAINAAGVGPASLPSDPATARDPIAPPGPPFPKVTDWTKSSADLEWSPPLKDGGSRVTGYIVEYKEEGKEEWEKAKDKEVRGTKLVVTGLKEGAFYKFRVRAVNIAGIGEPGEVTDAIEMKDRLELPDLQLDASVRDRIVVHAGGVIRIIAYVSGKPPPTVTWNMNERALPQEATIETTAISSSMVIKNCQRSHQGVYSLLAKNAAGERKKTIIVDVLDVPGPVGIPFLAHNLTNDSCKLTWFSPEDDGGSPITNYVIEKREADHRAWTPVTYTVTRHNATVQGLIQGKAYFFRIAAENSIGMGPFVETTDALVIRDPITVPERPEDLEVKEVTKESVTLTWNPPKYDGGSDIINYVLESRLIGTEKFHKVTSDNLMSRKYTVKGLKEGDTYEYRVSAVNIVGQGKPSFCTKPITCKDELAPPTLDLDFRDKLTIRVGEAFALTGRYSGKPKPKVSWFKDEADVLEDDRTHIKTTPTTLALEKLKAKRSDSGKYSVVVENSTGSRKGVCQVNVVDRPGPPVGPVIFDEVTKDYMVISWKPPLDDGGSEITNYIIEKKEVGKDVWMPVTSASAKTTCKVSKLLEGKDYIFRIHAENLYGISDPLVSDSMKAKDRFRVPDAPDQPIVTEVTKDSALVSWNPPHDGGKPITNYILEKRETMSKIWARVTKEPIHPYTKFRVPDLLEGCHYEFRVSAENEIGIGDPSPPSKPVFAKDPIAKPSPPVNPEAIDTTCNSVDLTWQPPRRDGGSKILGYIVEYQKVGDEEWKRANHTPESCPETNYKVTGLRDGQSYKFRVIAVNAAGESDPAHVPEPVLVKDRLEPPELILDANMAREQHIRVGDTLRLSAIIKGVPFPKVTWKKEDRAAPTKARIDVTPVGSKLEIRNAAHEDGGIYSLTVENPAGSKTVSVKVLVLDKPGPPRDLEVSEIRKDSCYLTWKEPLDDGGSVITNYVVERKDVASTEWSPLSTTSKKKSHLAKHLNEGNQYVFRVAAENQYGRGPFVETPKPIKAVDPLHPPGPPKNLHHVDVDKTEVSLVWNKPDRDGGSPITGYLVEYQEEGTPDWIKFKTVTNLACVVTGLQQGKTYRFRVKAENIVGLGLPDTTIPIECQEKLVPPSVELDVKLIEGLVVKAGTTVRFPAIIRGVPIPTAKWTTDGNEIKTDEHYTVETDNFSSVLTIKNCLRKDTGEYQITVSNAAGTKTVAVHLTVLDVPGPPTGPINILEVTPEYMTISWLPPKDDGGSPVINYIVEKKDTKKDTWGVVSSGSSKTKLKVPHLQKGYEYVFRVKAENKIGIGPPLDSVPTVAKHKFSPPSPPGKPVVTDITENAATVAWTLPKSDGGSPITGYYLERREVTGKWVRVNKTPLVDLKFRVTGLYEGNTYEFRVFAENLAGLSGPSPSSDPIKACRPIKPPGPPINPKLKDKTRESADLVWTKPLSDGGSPILGYVVECQKAGTTQWDRINKDELIRQCAFRVPGLIEGNEYRFRIKAANIVGEGEPRELAESVIAKDILHPPEVELDVTCRDVITVRVGQTIRILARVKGRPEPDITWSKEGKALVRDKRVNIIHELPRVELQIKEAVRADHGKYIISAKNSSGHAQGFAIVNVLDRPGPCQNLKVTNVTKENCTISWENPLDNGGSEITNFIVEYRKPNQKGWSIVASDVTKRLIKANLLANNEYYFRVCAENKVGVGPTIETKTPILAINPIDRPGEPENLHIADKGKTFVYLKWRRPDYDGGSPNLSYHVERRLKGAADWERVHKGSIKETHYMVDKCVENQIYEFRVQTKNEGGESDWVKTEEVVVKEDLQKPVLDLKLSGVLTVKAGDTIRLEAGVRGKPFPEVSWTKDKDATDLTRSPRVKIDTSGDSSKFSLTKAKRSDGGKYVVTATNTAGSFVAFATVNVLDKPGPIRNLKITDVCSDRCSLRWDPPEDDGGCEIQNYILEKCESKRMVWSTFSSTILTPGTTVTRLIEGNEYIFRVRAENKIGTGPPTETKPVIAKTKYDKPGRPDPPEVTKVSKEEMTVVWAPPEYDGGKSITGYYLEKKEKHSTRWVPVNKSAIPERRLKVQNLLPGHEYQFRVKAENEVGIGEPSLPSRPVVAKDPIEPPGPPTNLKVVDTTKSSITLGWGKPVYDGGAPIIGYVVEMRPKKADMSPDEGWKRCNAAAQLVRMEFTVTSLDENQEYEFRVCAQNQVGIGRPAELKDAIKPKEILEPPEIDLDASMRKLVTVRAGCPIRLFAIVRGRPAPKVTWRKVGIDNVVRKGQVDLVDTMAFLVIPNSTRDDSGKYSLTLVNPAGEKAVFVNVRVLDTPGPVSDLKVSDVTKTSCHISWAPPENDGGSQVTHYIVEKREAERKTWATVTPEVKKTSFHVTNLVPGTEYFFRVTAVNEYGPGVPTDVPKPVLATDPLSEPDPPRKLEVTEMTKNSAVLAWLPPLRDGGAKIDGYIVSYREEEQPADRWTEYSVVKDLSLVVTGLKEGKKYKFRVAARNAVGVSLPREAEGVYEAKEQLLPPKILMPEQITIKAGKKLRIEAHVYGKPNPVCKWKKGEDDVVTSSHLAVHKAENSSVLIIKDVTRKDSGYYSLTAENSSGTDTQKIKVIVMDAPGPPQPPFDISDIDADACSLSWHIPLEDGGSNITNYIVEKCDVSRGDWVTALASVTKTSCRVGKLIPGQEYIFRVRAENRFGISEPLTSPKMVAKFPFGVPSEPKNARVTKVNKDCIFVAWDRPDSDGGSPITGYLIERKERNSLLWVKANDTPVRSTEYPCAGLVEGLEYSFRIYALNKAGSSPPSKPTEYVTARTPVDPPGKPEVIDVSKSTVSLVWARPKHDGGSKIIGYFVEACKLPGDQWIRCNTSPHQIPQEEFTVTGLEENAQYQFRAIAKTAVNISQPSEPSDPVTIMAESVPPRIELSVAMKSLLTVKAGTNVCLDATVFGKPKPTVSWKKDGTLLKPSEGIKMAMKRNLCTLELFSVTRKDSGDYTITAENPSGSKSATIKLKVLDKPGPPASVKINKMYSDRAMLSWEPPLEDGGSEITNYIVDKRETSRPNWAQVSATVPITSCSVEKLIEGHEYQFRICAENKYGVGDPILTEPAIAKNPYDPPGRCDPPVISNVTKDHMTVSWKPPADDGGSPITGYLVEKRETHAVNWTKVNRKPVIERTIKATGLQEGTEYEFRVIAINKAGPGKPSDASKAVYAQDPLYPPGPPAFPKVYDTTRSSVSLTWGKPAYDGGSPIIGYLVEVKRADSDNWVRCNLPQKLQKTRFEVTGLMENTEYQFRVYAVNKIGYSDPSDVPDKHCPKDILIPPEGELDADLRKTLILRAGVTMRLYVPVKGRPPPKITWSKPNVNLRERVGLDIKSTDFDTFLRCEKVNKYDAGKYILTLENSCGKKEYTIVVKVLDTPGPPVNVTVKEISKDSAYITWDPPIIDGGSPIINYVVEKRDAERKSWSTVTTECSKTSFRVSNLEEGKSYFFRVFAENEYGIGDPGETRDAVKASETPGPVVDLKVTSVTKSSCSIGWKKPRSDGGSRIIGYVVDFLTEENKWQRVMKSLSLQYTTKDLTEGKEYTFRVSAENENGEGTPSEITAVAKDDVVAPDLDLKDLPDLCYLAKENSNFRLKIPIKGKPVPSVSWKKGEDPLATDTRVSVESSAVNTTLVVYDCRKSDAGKYTITLKNVAGTKEGTLTIKVVGKPGIPTGPIKFEEVTAEAVTLKWGPPKDDGGSEITNYILEKRDSVNNKWVTCASAVQKTTFRVMRLHEGMEYTFRVSAENKYGVGEGLKSEPIVARHPFDVPDAPPPPNIVDVRHDSVSLTWTDPRKTGGSPITGYHIEFKERNSLLWKRANKTPIRMKDFKVTGLTEGLEYEFRVMAINLAGVGKPSLPSEPVVALDPIDPPGKPEVISVTRNSVTLVWTEPKYDGGHKLTGYIVEKRELPSKSWTKANHVNVPDCAFTVTDLVEGGKYEFRVRAKNTAGAISAPSESTDTIICKDEYEAPTIVLDPTIKDGLTVKAGDTIILSAISILGKPLPKSSWSKAGKDIRPSDIVQITSTPTSSMIAVKYATRKDAGEYTITATNPFGTKSEHVKVTVLDVPGPPGPIEISNVSAEKATLTWTPPLEDGGSPIKSYVLEKRETSRLLWTVVAEDIQSCRHVATKLIQGNEYVFRVSAVNQYGKGEPVQSEPVKMVDRFGPPGPPGKPEVSNVTKNTATVSWKRPVDDGGSEITGYHVERREKKSLRWVRATKTPVSDLRCKVTGLQEGNTYEFRVSAENKAGIGPPSDASNPVLMKDVAYPPGPPSNARVTDTTKKSASLAWGKPHYDGGLEITGYVVEHQKVGDEGWIKDTTGTALRITEFVVPDLETKEKYNFRISAINDAGVGEPAVIPNVEIVEREMAPDFELDAELRRTLVVRAGLSIRIFVPIKGRPAPEVTWTKDNINLKNRANIENTESFTLLIIPECNRYDTGKFVMTIENPAGKKSGFVNVRVLDTPGPVLNLRPTDITKESVTLHWDLPLIDGGSRITNYIVEKREATRKSYSTVTTKCHKCTYKVTGLSEGCEYFFRVMAENEYGIGEPAETTEPVRASEAPSPPDSLNIMDITKSTVSLAWPKPKHDGGSKITGYVIEAQRKGSDQWTHITTVKGLECVVKNLTEGEEYTFQVMAVNSAGRSAPRESRPVIVKEQTMLPELDLRGIYQKLVIAKAGDNIKVEIPVLGRPKPTVTWKKGDQILKQTQRVNFENTATSTILNINECVRSDSGPYPLTARNIVGEVGDVITIQVHDIPGPPTGPIKFDEVSSDFVTFSWEPPENDGGVPISNYVVEMRQTDSTTWVELATTVIRTTYKATRLTTGVEYQFRVKAQNRYGVGPSITSAPVVANYPFKVPGPPGTPQVAAVTKDSITISWHEPLSDGGSPILGYHVERKERNGILWQTVSKALVPGNIFKSSGLTDGIAYEFRVIAENMAGKGKPSKPSEPILALDPIDPPGKPVPLNITRHTVTLKWAKPEYTGGFKITSYIVEKRDLPNGRWLKANFSNILENEFTVSGLTEDAAYEFRVIAKNAAGAISPPSEPSDAITCRDDIEAPRIMVDVKFKDTITLKAGEAFKLEADVSGRPPPTMEWTKDGKELENTAKLEIKIADFSTNLVNKDSLRRDGGAYTLTATNPGGFAKHIFHVKVLDRPGPPEGPLAVSEVTSEKCVLSWLPPLDDGGAKIEYYVVQKRETSRLAWTNVASEVQVTKLKVTKLLKSNEYIFRVMAVNKYGVGEPLDSEPVLAVDPYGPPDPPKNPEVTSITKDSMVVCWGHPDSDGGSEIINYIVERRDKAGQRWVKCNKKTLTDLRYKVSGLTEGHEYEFRVMAENRAGISAPSATSPFYKACDAVFKPGPPGNPRVLDTSRSSISIAWNKPIYDGGSEITGYMVEIALPEEDEWKIVTPPSGLKATSYTITNLTENQEYKIRIYAMNSEGIGEPALVPGTPKAEDRMLPPEIELDAELRKVVTIRACCTLRLFVPIKGRPAPEVKWTREHGESLDKASIESTSSYTLLIIGNVNRFDSGKYILTIENSSGSKSAFVNVRVLDTPGPPQDLKVKEVTKTSVTLTWEPPLIDGGSKINNYIVEKRESTRKAYSTVTTNCHKTSWKVDQLQEGCSYYFRVLAENEYGIGLPAETAESVKASERPLPPGKITLVDVTRNSVSLSWEKPEHDGGSRILGYIVEMQSKGSDKWVTCATVKVTEATITGLIQGEEYSFRVSAQNEKGISDPRQLSVPVIAKDLVIPPAFKLLFTTFTVLAGEDLKVDVPFTGRPTPAVTWHKDDVPLKQTTRVNAESTENNSLLTIKEACREDVGHYIVKLTNSAGEATETLNVIVLDKPGPPTGPVKMEEVTADSVTFSWGPPKYDGGSSINNYIVEKRDTSTTTWQIVSATVARTTIKACRLKTGCEYQFRIAAENRYGKSTYLTSEPVVAQYPFKVPGPPGTPFVTLSSKDSMEVHWNEPVSDGGSKVIGYHLERKERNSILWVKLNKTPIPQTKFKTTGLDEGIEYEFRVSAENIVGIGKPSKVSESYVARDPCDPPGRPEPIIVTRNSVTLQWKKPAYDGGSKITGYIVEKKELPDGRWMKASFTNVIDTQFEVTGLVEDHRYEFRVIARNAAGVFSEPSESTGAITARDEVEPPQIRMDPKYKDTIVVHAGELFKIDADIHGKPIPTTQWIKGDHELSNTARLEIKSTDFATSLSVKDAIRIDSGSYILKAKNVAGEKSVTVNVKVLDRPGPPEGPIVISGVTAEKCTLAWKPPLQDGGSDIINYIVERRETSRLVWTVVDANVQTLGCKVTKLLEGNEYIFRVMAVNKYGVGEPLESEPVTAKNPFVVPDAPKAPEITAVTKDSMIVVWERPAFDGGSEILGYVLEKRDKEGIRWTRCHKRLIGELRLRVTGLLENHNYEFRVSAENAAGLSEPSPPSAYQKACDPIYKPGPPNNPRVTDITRSSVFLSWGKPIYDGGCEIQGYIVEKCDTSVGEWTMCTPPTGINKTNIEVEKLLEKHEYNFRICAVNKAGVGEHADVPGPVIVEEKLEAPDIDLDLELRKILNIRAGGSLRLFVPIRGRPTPEVKWGKVDGEIRDAAIIDSTSSFTSLVLDNVNRYDSGKYTLTLENSSGTKSAFVTVRVLDTPSPPVNLKVTEITKDSVSITWEPPLLDGGSKIKNYIVEKREATRKSYAAVVTNCHKNSWKIDQLQEGCSYYFRVTAENEYGIGLPAHTDDPVKVAEVPQPPGKITVDDVTRNSVSLSWTKPEHDGGSKIIQYIVEMQAKHSEKWSECARVKSLEAVITNLTQGEEYLFRVVAVNEKGRSDPRSLAVPIVAKDLVIEPDVRPAFNSYSVQVGQDLKIEVPISGRPKPTITWTKDDLPLKQTTRINVTDSLDLTVLSIKETHKDDGGHYGITVANVVGQKTASIEIITLDKPDPPKGPVKFDEVSAESITLSWEPPLYTGGCQITNYVVQKRDTTTTVWEVVSATVARTTLKVTKLKTGTEYQFRIFAENRYGQSFALESEPIVAQYPYKEPGPPGTPFVTAVSKDSMVVQWHEPINNGGSPVIGYHLEKKERNSILWTKVNKTIIHDTQFKAVNLEEGIEYEFRVYAENIVGIGKASKNSECYVARDPCDPPGTPEAIIVKRHEITLQWTKPAYDGGSVITGYIVEKRDLPEGRWMKASFTNVIETQFTVSGLTEDQRYEFRVIAKNAAGAVSKPSDSTGPITARDEVELPRISMDPKFRDTIVVNAGETFRLEADVHGKPLPTIEWLRGDKEIEESARYEIKNTDFKALLIVKDAIRIDGGQYILRASNVAGSKSFPVNVKVLDRPGPPEGPVQVTGVTAEKCTLTWSPPLQDGGSDISHYVVEKRETSRLAWTVVASEVVTNSLKVTKLLEGNEYIFRIMAVNKYGAGEPLESAPVLMKNPFVLPGPPKSLEVTNIAKDSMTVCWNRPDSDGGSEIIGYIVEKRDRSGIRWIKCNKRRITDLRLRVTGLTEDHEYEFRVSAENAAGVGEPSPATLYYKACDPVFKPGPPINAHVVDTTKNSITLAWGKPIYDGGSEILGYVVEICKADEEEWQIVTPQTGLKATRFEISKLTEHQEYKVRVCALNKVGLGEATAVPGTVKPEDKLEAPELDLDSELRKGIVVRAGGSVRIHIPFKGRPTPEITWSREEGEFTDKVQIEKAANYTQLSIDNCDRNDAGKYVLKLENSSGSKSAFVTVKVLDTPGPPQNLAVKEVRKDSVLLVWDPPLIDGGAKVKNYVIDKRESTRKAYANVSNKCSKTSLKVENLTEGAIYYFRVMAENEFGIGVPVETVDAVKASEPPSPPGKVTLTDVSQTSASLMWEKPEHDGGSRILGYVVEMQPKGTEKWSVVAESKVCSAVVTGLSSGQEYHFRVKAYNEKGQSDPRVLGVPVIAKDLTIQPSFKLPFNTYSVQAGEDLKIEIPVIGRPRPEISWVKDGEPLKQTTRVNVEKTATSTILHIKESNKDDFGKYTVTATNSAGTATENLSIIILEKPGPPVGPVRFDEVSADFVVISWEPPAYTGGCQISNYIVEKRDTTTTTWHMVSATVARTTIKITKLKMGSEYQFRIFAENRYGKSAPLDSKPVIVQYPFKEPGPPGTPFVTSVSKDQMLVQWHEPVNDGGSKVIGYHLEQKEKNSILWVKLNKTPIQDTKFKTTGLDEGLEYEFRVSAENIVGIGKASKVSECFVARDPCDPPGRPEAIVITRNNVTLKWKKPAYDGGSKITGYIVEKKDLPDGRWMKASFTNVLDTEFTVSGLVEDQRYEFRVIARNAAGNFSEPSESTGAITARDEIDAPNASLDPKYKDVIVVHAGETFVLEADIRGKPIPDIVWSKDGRELEETAARMEIKSTIQRTTLVVKDCIRSDGGQYILKLSNVGGTKTIPITVKVLDRPGPPEGPLKVSGVTAEKCYLAWNPPLQDGGASISHYIIEKRETSRLSWTQVSTEVQALNYKVTKLLPGNEYIFRVMAVNKYGTGDPLESEPVIARNPYKPPGPPSPPEVSAITKDSMVVTWARPVDDGGAEIEGYILEKRDKEGVRWTKCNKKTLTDLRFRVTGLTEGHSYEFRVAAENAAGVGEPSEPSVFSRACDALYPPGPPSNPKVTDTSRSSVSLAWNKPIYDGGAPVKGYVVEVKEATADEWTTCTPPTGLQGKQFTVTELKENTEYNFRICAINSEGVGEPATIPGSVAAKERQEPPEIELDADLRKVVILRASATLRLFVTIKGRPEPEVKWEKAEGVLTDRAQIEVTSSYTMLVIDNVTRFDSGRYNLTLENNSGSKTAFVNVRVLDSPSAPVNLTVREVKKDSVILAWEPPLIDGGAKITNYIVEKRETTRKAYATVTNNCTKNSFKIENLQEGCSYYFRVLASNEYGIGLPAETTEPVKASEPPLPPGRVTLVDVTRNTATIKWEKPESDGGSKITGYVVEMQTKGSEKWSICTQVKTLEATISGLTAGEEYIFRVAAINEKGKSDPRQLGVPVIARDIEIKPSVELPFNTFNVKARDQLKIDIPFKGRPQATVSWKKDGQTLKETTRVNVSSSKTVTSLIIKEASREDVGTYELCVSNSAGSVTVPITVIVLDKPGPPGPIHIDEVSCDNITISWNPPEYDGGCQISNYIVEKRETTSTTWHVVSQAVARTSIKIVRLVTGSEYQFRVCAENRFGKSSYSESSAVVAEYPFSPPGPPGTPKVVHATKSTMLVTWQVPVNDGGSRVLGYHLEYKERSSILWSKANKTLIADTQMKVSGLDEGLMYEYHVYAENIAGIGKCSKSCEPVPARDPCDPPGQPEVTNITRKSVSLKWSKPHYDGGAKITGYIIERRELPDGRWLKCNFTNVPETYFEVTELTEDQRYEFRVFARNAADSISEPSESTGPITVKDDVEAPRIMMDVKFRDVIVVKAGEVLKINADVAGRPLPIISWAKDGVEIEERARTEIVSTDYTTLLTVKDCVRRDSGQYVLTVKNVAGTRSMAVNCKVLDKPGPPAGPLEITGLTAEKCSLSWGPPQEDGGAAIDYYIVEKRETSRLAWTICEGELRTTFCKVTKLLKGNEYIFRVTGVNKYGVGEPLESMAVKALDPFTVPSPPTSLEITSVTKEFMTLCWSRPESDGGSEISGYIVERREKNSLRWVRVNKKPVYDLRVKSTGLREGCEYEYRVYAENAAGLSLPSESSPLIRAEDPVFLPSPPSKPNVMDSGKTNITIGWVKPLFDGGAPITGYTVEFKKSDETDWQTAIQNLRGTEYTVSGLTTGAEYVFRVRSINKVGASDPSDSSDPQIAKEREEEPVFDLDTEMRKTLIVKAGASFTMTVPFRGRPVPSVSWSKPDTDLRTRAYIDSTESRTSLTIENANRNDSGKYTLMIQNVLNAASLTLVVKVLDSPGPPANITVHDVTKESAVLSWDVPENDGGAPVKNYHIEKREASKKAWVSVTNNCNRLSYKITNLQEGAIYYFRVSGENEFGVGVPAETKEGVKITEKPSPPEKLGVTSVSRDSVSLAWLKPEHDGGSRIVHYVIEALEKGQTTWMRCAVVKSTHHVVSGLRQNSEYFFRVFAENQAGLSDPRELLLPVLIKEQLEPPEIDMKNFPSHTVYVRAGSNLKVDIPISGKPLPKVTLSRDGVPLKATMRFNTEITAENLTINLKESVAADAGRYEITAANSSGTTKAFINIVVLDRPGPPTGPVVISDITEDSVTLKWEPPKYDGGSQVTNYIVLKRETSTAVWTEVSATVARTMIKVMKLTTGEEYQFRIKAENRFGISDHIDSACVVVKLPYTTPGPPSTPWVTNVTRESITVGWHEPVSNGGSPVTGYHLEMKDRNSILWQKANKMVIRTTHFKVTTISAGLIYEFRVYAENAAGIGKPSHPSEPVLAIDACEPPRNVRITDISKNSVNLSWQQPAFDGGSKITGYMVERRDLPDGRWAKASFTNVTETHFTVSGLTQNAQYEFRVFARNAVGSISNPSEVVGPITCIDSYGGPVIDLPLEYTEVVKYRAGTSVKLRAGISGKPEPTIEWYKDDKELQTNALVCVENTTDLASILIKDATRLNSGTYELKLRNALGSASATIRLQILDKPGPPGGPIEFKTVTAEKITLLWQPPADDGGAKITHYIVEKRETSRVVWSMVSENLEECIIKTTKIIKGNEYIFRVRAVNKYGIGDALESHPVIARNAFVTPGPPSVPEVTKINKNSMTVVWSRPVADGGSDISGYILEKRDKKSLGWFKVLKETIRDTRQKVTGLTEHSDYQYRVCAVNAAGQGPFSEPSDFYKAADPIDPPGPPAKIRIADSTKSSITLGWSKPVYDGGSAVTGYVVAMRQGEEEEWTVVSTKGEVRTTEYVVSNLSPGVNYYFQVSAVNCAGQGEPIAMTEPVQAKDILEEPEIDLDVAFRTSIIAKAGEDVHALVPFKGRPPPTVTWRKDEKNLGSDARYSIQNTDSSSILTIPQVTRHDTGKYILTIENGVGQPKSSTVSVKVLDTPAACQNLQVKHVSQGTVTLFWDPPLIDGGSPIINYIIEKKDATKRTWSSVSHKCSSTSFKVTDLSEKTPFFFRVFAVNEIGIGEPCETTEPVKAAEVPAPIRDLSVKDSTKTSVTLSWTKPDFDGGSVITDYIVERKGKGEQTWSHAGISKTCEIEVGKLKELSELEFRVSARNEKGLSDSVSIGPVTVKELVIPPEVDLSEIPGAQVAVRIGHNVHLELPYKGKPKPSISWLKDGLPLKESELVRLGKTENKLTLSIKNAKKENGGKYTIILDNAVCRNTYPITVITLGPPSKPKGPIRFDEIKADSVIMSWDVPDDDGGGEITCYSIEKREASQTNWKMVCSSVARTTFKVPNLVKDAEYQFRVRAENRYGVSQPLVSNIILAKHQFRIPGPPGKPVTYNVTSDGMSLTWDAPVYDGGSEVTGYHVEKKERNSILWQRINMSPISGREYRATGLMEGLDYQFRVFAENSAGLSSPSDPSKFTLAVSPVDPPGTPDYIDVTRETITLKWNPPLRDGGSKIVAYSIEKRQGSDRWTRCNFTDVSECQYTVTGLSPGDRYEFRIIARNAVGTISPPSQSSGVIMTRDENVAPTVEFGPEYFDGITIKSGESLRIKALVQGRPVPRVTWFKDGVEIEKKMNMEITDVLGSTSLFVRDATRDHRGVYRVEAKNASGSTKAEITVRVQDTPGKPVGPIRFTNITGEKMTLWWDAPHNDGCAPVSHYLIEKRETSRLAWALIEDNCEALSYTATKLITGNEYQFRISAVNKFGVSRPLDSDPVVAQIQYTIPDAPGTPEPSNVTGNSITLTWARPESDGGSEIQQYILERREKKSTRWVKVISKRPICETRFKVTGLTEGNEYEFHVMAENAAGVGPASGVSRLIKCREPVNPPGAPTVVKVTDTSKTTVSLEWSKPVFDGGLEIIGYIIEMCKADLGDWHKVNVEACVKTRYTVTDLQAGEEYKFRVSAINAAGKGDSCEVTGTIKAVDRLSAPELDIDANFKQTHVVRAGASIRLFIAYQGRPTPTAVWSKPDSNLSIRADIHTTDSFSTLTVENCNRTDAGKYTLTVENNSGSKSITFTVKVLDSPGPPGPITFKDVTRGSATLMWDAPLLDGGARIHHYVVEKREASRRSWQVVSEKCIRQILRVSDLLEGVPYYFRVSAENEYGVGEPYEMPEPMVATEQPAPPRRLDIVDTSKSSVVLAWLKPDHDGGSRITGYLLEMRQKGSDFWVEAGHTKQMTFTVERLVENTEYEFRVKAKNDAGYSEPREAFSSVIIKEPQIEPTADLTGITNQLITCKAGSTFTIDVPISGRPAPKVTWKLEEMRLKETDRVSITTTKDRTTLSVKDSMRGDSGRYFLTLENTAGVKTFTITVVVIGRPGPVTGPIEVSSISAESCVLSWAEPQDNGGTDITNYIVEKRESGTTAWQLVNSSVKRTQIKVTHLTKYMEYSFRVSSENRFGVSKPVESAPIVAEHPFVPPSAPTRPEVYYVSANAMSIRWEEPYHDGGSKVIGYWVEKKERNTILWVKENKVPCLECNYKVTGLVEGLEYQFRTYALNAAGVSKASEASRPVMAQNPVDAPGRPEVTDVTRSTVSLIWSAPVYDGGSKVVGYIIERKPVSEVGDGRWLKCNYTIVSDNFFTVTALSEGDTYEFRVLAKNAAGVISKGSESTGPITCRDEYAPPKAELDARLQGDLVTIRAGSDLVLDAAVGGKPEPKIIWTKGDKELDMCEKISLQYTGKRATAVIKFCDRSDSGKYTLTVKNASGTKAVSVLVKVLDSPGPCGKLTVSRVTEEKCTLAWSLPQEDGGAEITHYIVERRETSRLNWVIVEGECPTLSHVVTRLIKNNEYIFRVRAVNKYGPGVPVESEPIVARNSFTIPSQPGIPEEVGAGKEHIIIQWTKPESDGGNDISNYLVDKREKKSLRWTRVNKDHVVYDTRLKVTGLMEGCDYQFRVTAVNAAGNSEPSEASNFISCREPSYTPGPPSAPRVVDTTKHSISLAWTKPMYDGGTDIIGYVLEMQEKDTDQWYRVHTNATIRNNEFTVTDLKMGQKYSFRVAAVNVKGMSEYSESTAEIEPVERLEIPDLELADDLKKTVTIRAGASLRLMVSVSGRPPPVITWSKKGIDLASRAIIDTTESYSLLIVDKVNRYDAGKYTIEAENQSGKKSATVLVKVYDTPGPCPSVKVKEVSRDSVTITWEVPTIDGGAPVNNYIIEKREAAMRAFKTVTTKCSKTLYRISGLIEGAMYYFRVLPENIYGIGEPCETSDAVLVSEVPLVPTKLEVVDVTKSTVTLAWEKPLYDGGSRLTGYVLEACRAGTERWMKVVTLKPTVLEHTVISLNEGEQYLFRVRAQNEKGVSEPREIVTAVTVQDLRVLPTIDLSTMPQKTIHVPAGRPVELVIPIAGRPPPAASWFFAGSKLRESERVTVETHTKVAKLTIRETTIKDTGDYVLELKNVTGTTSETIKVIVLVDKPGPPSGPIKVDEIDATSVTISWGPPELDGGAPLSGYVVEQRDAHRPGWLPVSESVTRSTFKFTRLIEGNEYVFRVAATNRFGIGSYLQSEVIECRSSINIPGPPETLQIFDVSREGMTLTWYPPEDDGGSQVTGYIVERKEVRADRWVRVNKVPVTMTRYRSTGLIEGLEYEHRVTAVNVRGTGKPSRPSKPTVAMDPIAPPGKPQNPRVTDTTRTSVSLAWSVPEDEGGSKVTGYLIEMQKVDQHEWTKCNTTPTKIREYTLTHLPQGAEYRFRVLACNAGGPGEPAEVPGTVKVTEMLEYPDYELDERYQEGILVRQGGVIRLTIPIKGKPFPICKWTKEGQDISKRAMIATSETHTELVIKEADRDDSGTYDLILENKCGKKAVYIKVRVIGNPNTPEGPLEYDDIQARSVRVSWRPPADDGGADILGYILERREVPKSAWYTIDSRVRGTSLVVKGLKENVEYHFRVSAENQFGISKPLKSEEPVIPKTPLNPPEPPSNPPEILDVTKSSVSLSWSRPKDDGGSRVTGYYIERKETSTDKWVRHNKTQITTTMYTVTGLVPDAEYQFRIIAQNDVGLSETSPASEPVVCKDPFDKPSQPGELEILSISKDSVTLQWEKPECDGGKEILGYWVEYRQSGDSAWKKSSKDRIKDRQFTIGGLLEATEYEFRVFAENETGLSRPRRTAMSVKTKLTSGEAPGVRKEMKDVTTKLGEAAQLSCQIVGRPLPDIKWFRFGKELVQSRKYKMSSDGRTHTLTVMTEEQEDEGVYTCVATNEVGEVESSSKLLLQATPQFHPGYPLKEKYYGAVGSTLRIHVMYIGRPVPAITWFRGQKLLQNSENITIENTEHYTHLVMKNVQRKTHAGKYKVQLSNVLGTVDTMLDVEIQDKPDKPTGPIVIEALLKNSVVISWKPPADDGGSWITNYVVEKCEAKEGAEWQLVSSAISVTTCRIVNLTENAGYYFRVSAQNMFGISEPLEVSSVVIIKSPFEKPGAPGKPTITAVTKDSCVVAWKPPASDGGAKIRNYYLEKREKKQNKWIAVTTDEIRETVFSVQNLIEGLEYEFRVKCENLGGESEWSEISEPVTPKSDVPIQAPHFKEELRNLNVRYQSNATLVCKVTGHPKPIVKWYRQGKEIIADGLKYRIQEFKGGYHQLIIASVTDDDATVYQVRATNQGGSVSGTASLEVEDTVPAKIHLPKNLEGMGAVHALRGEVISIKIPFSGKPDPVITWQKGQDLIDNNGHYQVIVTRSFTSLVFPNGVERKDAGFYVVCAKNRFGIDQKTVELDVADVPDPPRGVKVSDVSRDSVNLTWTEPASDGGSKVTNYIVEKCATTAERWIRVGQARETRYTVINLFGKTSYQFRIIAENKFGLSKPSEPSEPTVTKEDKTRAMNYDEEVDETREVSMTKASHSSTKELYEKYMIAEDLGRGQFGIVHRCVETSSKKTYMAKFVKVKGTDQVLVKKEISILNIARHRNMLYLHESFESMEELVMIFEFISGLDIFERINTSAFELNEREIVSYVRQVCEALEFLHSHNIGHFDIRPDNIIYQTRRSSTIKIIEFGQARQLKPGDNFRLQFTAPEYYAPEVHQHDVVSTATDMWSLGTLVYVLLSGINPFLAETNQQIIENIMNAEYTFDEEAFQEISLEAMDFVDRLLVKERKSRMTASEALQHPWLKQKTERVSTKVIRTLKHRRYYHTLVKKDLNMVVSAARISCGGAIRSQKGVSIAKVKVASIEIGPVSGQIMHAVGEEGGYVKYVCRIENYDQSTQVTWYFGVRQLENSEKYEITYEDGVATMYVKDITKFDDGTYRCKVVNDYGEDSSYAELFVKGVREVYDYYCRRTVKKLKRRTDAMRLLERPPEFTLPLYNKTAYVGENVRFGVTITVHPEPRVTWYKSGQKIKPGDDDRKYIFESDKGLYQLTINSVTMDDDAEYTVVARNKYGEDSCKAKLTVTPHPPPSDTTLRPMFKRLLANAECQEGQSVCFEIRVSGIPPPTLKWEKDGRPLSLGPNIEIIHEGLDYYALHIRDTLPEDTGYYRVTATNTAGSSSCQAHLQVERLRYVKQEFKTKEEHERHVQRQIDKTLRMAEILSGTEAVPLTQVAQEALREAAILYKPAVSTKTVKGEYRLETEEKKEERKLRMPYEVPEPRKYKQTTIEEDQHIKQFVPMSDMKWYKKIRDQFEMPGKIDRVVQKRPKRIRLSRWEQFYVMPLPRITDQYRPKWRIPKLSQDDLEMVRPARRRTPSPDYYYYYRPRRRSLGDISDEELLLPIDDYLAMKRTEEERLRLEEELELGFSASPPSRSPPRFELSSLRYSSPQAHVKVEEARRDFRYSTYHIPTKEETSTSYAELRERHARASHRQAHQRQRIMAEREDHELLRPATTTQRLLEYKSELDHLAKEAKSRKKSRRQREVTEITEIEEEYEISKHAQRETSSSVSRLLRRRRSLSPTYIELMRPVSELIRPRPRPAEEYEDEEEEEEEDVERRSPTPERTRPRSPSPVSSERSLSRFERSARFDIFSRYESMKAALKTQKTSERKYEVLSQQPFTLDHAPRITLRMRSHRVPCGQNTRFILNVQSKPTAEVKWYHNGVELQESSKIHYTNTSGVLTLEILDCHIDDSGTYRAVCTNYKGEASDYATLDVTGGDYTTYASQRRDEQVPRSVFPELTRTEAYAVSSFKKTSEMEASSSVREMKSLMTETRESLSSYEHHASAEMKSAAIEEKSLEEKSTHRKIKTTLAARILTKPRSITVYEGESARFSCDTDGEPVPTVTWLRGGQVISTSARHQVTTSKYKSTFEISSVQASDEGNYSVVVENSDGRQEAQFTLTVQKARVTEKAVTSPPRVKSPEPRVKSPEGAKSPKRVKSPEPVTPHPKAVPPTETKPTPTEKVQQKPVAAPPKIIQSLKAEASKDIAKLTCVVESSALCAKEVTWYKDGRRLKENGHFQFHYSADGTYELKILNLAESDRGEYVCEVSGEGGTSKTNFQFVGQAFKSIHEQVSSISETNKKAAPKTAEPTETKKTEPKAPAPISTKPVIVTGLQDTTVSSDSVAKFAVKVTGEPQPTVIWTKDGKAISQGGKYKLSEDKGAFFLEIHKTDTSDSGLYTCTITNSAGSVSSSCKLTIKVVEDTEIQKVSAQKTSEITSQKKASVQEEISKKALISEEIKTSEVKSHEKLALKEEASTVLISEKVKKSEAASLEKSVVHEEITKTSQASEEIRTHAEIKAFSTQMSITAGQKVTLKANIAGATDVKWVLNGVELSNSDEYRYGISGSDQTLTIRQAGHKDEGILTCIGKTSQGIIKCQYDLTLSKELSDAPAFISQPRSQNVNEGQNVLFSCEISGEPSPEIEWFKNNLPISISSNISVSRSRNVYSLEIRNASVSDSGKYTIKAKNFHGQCSATASLTVLPLVEEPPREVVLRTSGDTSLQGSFSSQSVQMSASKQEASFSSFSSSSASSMTEMKFASMSAQSMSSMQESFVEMSSSSFMGKSSMTQLESSTSRMLKAGLRGIPPKIEALPSDISIDEGKVLTVACAFTGEPTPEITWSRGGRTIHDQEQRGRFMENTDDL

SEQ ID NO: 47 - protein, 265 aa, Gallus gallus
MSEAEEEYEEEQPEEEEEAAAAAEEEEEEEAEASKPHEEPEEERPRPRPVVPQLAPPKIPEGERVDFDDIHRKRMEKDLLELQTLIDAHFEQRRREENELVALMERIERRRAERNEQLRSRTEKERERQARLAEEKLRKEEEEAKKRAEDDAKKKKVLSNMPHFGGYLAKAEQRRGKRQTGREMKLRILAERKKPLHIEHMREDELRAKAKELHDWIQQLESEKFDLMEKLRRQKYEINVLYNRISHAQKFKKVVGKGRVGGRWK

SEQ ID NO: 48 - protein, 235 aa, Gallus gallus
AAEASKPHEEPEEERPRPRPVVPQLAPKIPEGERVDFDDIHRKRMEKDLLELQTLIDAHFEQRRREENELVALMERIERRRAERNEQLRSRTEKERERQARLAEEKLRKEEEEAKKRAEDDAKKKKVLSNMPHFGGYLAKAEQRRGKRQTGREMKLRILAERKKPLHIEHMREDELRAKAKELHDWIQQLESEKFDLMEKLRRQKYEINVLYNRISHAQKFKKVVGKGRVGGRWK

SEQ ID NO: 49 - protein, 188 aa, Gallus gallus
MPEPRERKSKITASRKLLLKSLMLAKAKEEWEQEIVDKQSEKERYLSERITPLHTSGLSLSQLQDLCRELHEKVEIVDEERYDIEAKCNHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTGQ

SEQ ID NO: 50 - protein, 166 aa, Gallus gallus
MLAKAKEEWEQEIVDKQSEKERYLSERITPLHTSGLSLSQLQDLCRELHEKVEIVDEERYDIEAKCNHNTREIKDLKLKVLDLRGKFKRPPLRRVRVSADAMLRALLGSKHKVSMDLRANLKSVKKEDTEKERPVEVGDWRKNVEAMSGMEGRKKMFDAAKSPTGQ

SEQ ID NO: 51 - protein, 223 aa, Salmo salar
MSDTEEVEAQKPQFKVPKIPDGDKVDFDDIQKKRQNKDLIELQALIDAHFEHRKKEEEELIALKERIEKRRAERAEQNRIRSEKEKERAARREEERLKREEADAKKKADEDAKKKSALSSMGSNYSSHLQKADSKRGGKKETEREKKKKILAGRRKALNIDHLNEEKLKEKAKELHEWMQTLESEKFDHIERLKRQKYEVTTLRKRVEELSKFSKKGKTVRRK

SEQ ID NO: 52 - protein, 287 aa, Gallus gallus
MSDTEEVEHGEAHEAEEVHEEAHHEEAHHEEAHHEEAHHAEAHHAEAHHEEAHAHAEEVHEPAPPPEEKPRIKLTAPKIPEGEKVDFDDIQKKRQNKDLIELQALIDSHFEARRKEEEELVALKERIEKRRAERAEQQRIRAEKEKERQARLAEEKARREEEDAKRKAEDDLKKKKALSSMGASYSSYLAKADQKRGKKQTARETKKKVLAERRKPLNIDHLNEDKLRDKAKELWDWLYQLQTEKYDFAEQIKRKKYEILTLRCRLQELSKFSKKAGAKGKVGGRWK

SEQ ID NO: 53 - protein, 603 aa, Sus scrofa
MASAPTSKYNSHSLENESIKRTSRDGVNRDMGEAVPRLPGETPITDKEVIYICPFNGPIKGRVYITNYRLYLRSLETDSALILDVPLGVISRIEKMGGATSRGENSYGLDITCKDMRNLRFALKQEGHSRRDMFEILTRYAFPLAHSLPMFAFLNEEKFNVDGWTVYNPVEEYRRQGLPNHHWRITFINKCYELCDTYPALLVVPYRAADEDLRRVATFRSRNRIPVLSWIHPENKTVIVRCSQPLVGMSGKRNKDDEKYLDVIRETNRQVNKLTIYDARPNVNAVANKATGGGYESDDAYHNAELFFLDIHNIHVMRESLKKVKDIVYPNVEESHWLSSLESTHWLEHIKLVLTGAIQVADRVSSGKSSVVVHCSDGWDRTAQLTSLAMLMLDSFYRSIEGFEILVQKEWISFGHKFASRIGHGDKNHADADRSPIFLQFIDCVWQMSKQFPTAFEFNEHFLITILDHLYSCRFGTFLYNCESARERQKVTERTVSLWSLINSNKDKFKNPFYTKEINRVLYPVASMRHLELWVNYYIRWNPRIKQQQPNPVEQRYMELLALRDEYIKRLEELQLANSAKLSEPAAAPSSPSQMVSHVQTHF

SEQ ID NO: 54 - protein, 298 aa, Sus scrofa
MPLSGTPAPNKKRKSSKLIMELTGGGQESSGLNLGKKISVPRDVMLEELSLLTNRGSKMFKLRQMRVEKFIYENHPDVFSDSSMDHFQKFLPTVGGQLGTAGQGFSYSKGSSGGQAGGSSSAGQYGSGQQHHHQGSGSGSGGAGGPGSQTGRGGDAGTTGVGETGTGDQAGGEGKHITVFKTYISPWEKAMGVDPHQKVELGIDLLAYGAKAELPQYKSFNRTAMPYGGYEKASKRMTFQMPKFDLGPLLSEPLVLYNQNLSNRPSFNRTPIPWLSSGEPVDYNVDIGIPLDGETEEL

SEQ ID NO: 55 - protein, 284 aa, Gallus gallus
MEAIKKKMQMLKLDKENAIDRAEQAEADKKQAEDRCKQLEEEQQGLQKKLKGTEDEVEKYSESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESKCGDLEEELKIVTNNLKSLEAQADKYSTKEDKYEEEIKLLGEKLKEAETRAEFAERSVAKLEKTIDDLEDEVYAQKMKYKAISEELDNALNDITSL

SEQ ID NO: 56 - protein, 434 aa, Bos taurus
MAMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNPELILPVPAFNVINGGSHAGNKLAMQEFMILPVGASSFREAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKNFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 57 - protein, 603 aa, Bos taurus
MASAPTSKYNSHSLENESIKRTSRDGVNRDVGETLPRLPGEIRITDKEVIYICPFNGPIKGRVYITNYRLYLRSLETDSALILDVPLGVISRIEKMGGATSRGENSYGLDITCKDLRNLRFALKQEGHSRRDMFEILTRYAFPLAHSLPIFAFLNEEKFNVDGWTVYNPVEEYRRQGLPNHHWRITFINKCYKLCDTYPALLVVPYRASDEDLRRVATFRSRNRIPVLSWIHPENKTVIVRCSQPLVGMSGKRNKEDERYLDVIRETNRQVNKLTIYDARPNVNAVANKATGGGYESDDVYHNAELFFLDIHNIHVMRESLKKVKDIVYPNVEESHWLSSLESTHWLEHIKLVLTGAIQVADRVSSGKSSVVVHCSDGWDRTAQLTSLAMLMLDSFYRSIEGFEILVQKEWISFGHKFASRIGHGDKNHADADRSPIFLQFIDCVWQMSKQFPTAFEFNERFLITILDHLYSCRFGTFLYNCESAREKQKVTERTVSLWSLINSNKDKFKNPFYTKEINRVLYPVASMRHLELWVNYYIRWNPRIKQQQPNPVEQRYMELLALRDEYIKRLDELQLANSAKLSDPSASPSSPSQMMPHVQTHF

SEQ ID NO: 58 - protein, 224 aa, Bos taurus
MELYETSPYFYQEPHFYDGENYLPVHLQGFEPPGYERAELSLSPEARVPLEDKGLGPAEHCPGQCLPWACKVCKRKSVSVDRRRAATLREKRRLKKVNEAFEALKRSTLLNPNQRLPKVEILRSAIQYIERLQALLSSLNQEERDLRYRGGGGPQAAVPSECSSHSASCSPQWGSALEFGPNPGDHLLPADPTDAHNLHSLTSIVDSITVEDVAAAFPDETIPN

SEQ ID NO: 59 - protein, 224 aa, Sus scrofa
MELYETSPYFYQEPHFYDGENYLPVHLQGFEPPGYERTELSLSPEARVPLEDKGLGTPEHCPGQCLPWACKVCKRKSVSVDRRRAATLREKRRLKKVNEAFEALKRSTLLNPNQRLPKVEILRSAIQYIERLQALLSSLNQEERDLRYRGGGGPQPGVPSECSSHSASCSPEWGSALEFGPNPGDHLLTADPTDAHNLHSLTSIVDSITVEDVAVAFPDETMPN

SEQ ID NO: 60 - protein, 2058 aa, Gallus gallus
MGGSGAAATLALGLALGLALGGWANCPERELQRREEEANVVLTGTVEEIMNVDPVHHTYSCKVRVWRYLKGKDIVTHEILLDGGNKVVIGGFGDPLICDNQVSTGDTRIFFVNPAPQYMWPAHRNELMLNSSLMRITLRNLEEVEHCVEEHRKLLADKPNSYFTQTPPTPRDACRGMLCGFGAVCERSPTDPSQASCVCKKTACPVVVAPVCGSDYSTYSNECELEKAQCNQQRRIKVISKGPCGSKDPCAEVTCSFGSTCVRSADGQTAGCVCPASCSGVAESIVCGSDGKDYRSECDLNKHACDKQENVFKKFDGACDPCKGILNDMNRVCRVNPRTRRVELLSRPENCPSKREPVCGDDGVTYASECVMGRTGAIRGLEIQKVRSGQCQHQDKCKDECKFNAVCLKRWHARCSCDRITCDGTYRPVCARDSRTYSNDCERQKAECHQKAAIPVKHSGPCDLGTPSPCLSVECTFGATCVVKNREPVCECQQVCQGRYDPVCGSDNRTYGNPCELNAMACVLKREIRVKHKGPCDRCGKCQFGAICEAETGRCVCPTECVPSSQPVCGTDGNTYGSECELHVRACTQQKNILVAAQGDCKSCGTTVCSFGSTCVGGQCVCPRCEQQPLAQVCGTDGLTYDNRCELRAASCQQQKSIEVAKMGPCEDECGSGGSGSGDGSECEQDRCRHYGGWWDEDAEDDRCVCDFTCLAVPRSPVCGSDDVTYANECELKKTRCEKRQNLYVTSQGACRALTTTPPPLPVVHCSQTIYGCCPDNMTLALGVGAAGCPSTCQCNPYGSYGGTCDPATGQCSCKPGVGGLKCDRCEPGFWNFRGIVTDSKSGCTPCNCDPVGSVRDDCEQMTGLCSCKTGITGMKCNQCPNGSKMGMAGCEKDPSAPKSCEEMSCEFGATCVEVNGFAHCECPSPLCSEANMTKVCGSDGVTYGDQCQLKTIACRQGQLITVKHVGQCHESITHTSHTMPPTPLPTLPLDKLIVPPPLQLTTQAPEPTELATTSLLMEASPTTRSHPTTRRVTTTRPVTTPWMTHGVLKTTVRPLSTSPVVLATTQPPYAESGSAEGSGDQEMSISGDQESSGAGSAGEEEVEESQVTPTPAIERATCYNTPLGCCSDGKTAAADAEGSNCPATKVFQGVLILEEVEGQELFYTPEMADPKSELFGETARSIESALDELFRNSDVKNDFKSIRVRDLGQSSAVRVIVESHFDPATSYTAADVQAASLKQIRASKKRTILVKKPQQEHVKFMDFDWIPRIFTTTITTTTATTMAPATTRRHTTASAATTAHILRQDTVGHPSAKLAAPASTRRPTSTLPTTARRKPTRQPPSTTKKPSRPCDSHPCLHGGTCEDDGREFTCRCPAGKGGAVCEKPIRYFIPSFGGKSYLAFKMMKAYHTVRIAMEFRATELSGLLLYNGQNRGKDFISLALVGGFVELRFNTGSGTGVITSKVRVEPGKWHQLVVNRNRRSGMLAVDGEHVSGESPTGTDGLNLDTDLFVGGAPEDQMAVVAERTAATVGLKGSIRLLDVNNQMYDLREKGSDVLYGSGVGECGNDPCHPNPCHHGASCHVKEAEMFHCECLHSYTGPTCADERNPCDPTPCHISATCLVLPEGGAMCACPMGREGEFCERVTEQDHTMPFLPEFNGFSYLELNGLQTLFLTCRQMSMEVVFLAKSPSGMIFYNGQKTDGKGDFVSLALHDGYLEYRYDLGKGAAVLRSKEPVPLNTWISVLLERSGRKGVMRINNGERVMGESPVPHAFLNLKEPFYVGGAPDFSKLARAAAISTSFYGAVQRISIKGVPLLKEQHIRSAVEISTFRAHPCTQKPNPCQNGGTCSPRLESYECACQRGFSGAHCEKVIIEKAAGDAEAIAFDGRTYMEYHNAVTKSEKALQSNHFELSIKTEATQGLILWSGKGLERSDYIALAIVDGFVQMMYDLGSKPVVLRSTVPINTNHWTHIKAYRVQREGSLQVGNEAPITGSSPLGATQLDTDGALWLGGMERLSVAHKLPKAYSTGFIGCIRDVIVDRQELHLVEDALNNPTILHCSAK

SEQ ID NO: 61 - protein, 166 aa, Gallus gallus
MASGVTVNDEVIKVFNDMKVRKSSTPEEIKKRKKAVLFCLSDDKKQIIVEEAKQILVGDIGDTVEDPYTAFVKLLPLNDCRYALYDATYETKESKKEDLVFIFWAPESAPLKSKMIYASSKDAIKKKFTGIKHEWQVNGLDDIKDRSTLGEKLGGNVVVSLEGKPL

SEQ ID NO: 62 - protein, 416 aa, Gallus gallus
MESYKVMLNGPAPWGFRLQGGKDFSMPLSISRLTPGGKAAQAGVGVGDWVLYIDGESTGTMTHIEAQNRIRACGDRLCLTLSRAQNHLGKPQKDSLPCSEPPKYNFAPSTALNKTARPFGASSPPNPRPGLVTKPVTYVPLAPACTPQHNGQVSVPDPSPGAAMKTEPGLAPRTPAATPGPTSRPPWAVDPSFAERYAPDKTSTVLSKHSQPATPTPMQNRSSIVQAAQQAPESPGRTPLCYKCNKIIRGRYLVALGHYYHPEEFTCCQCRKVLDEGGFFEEKGSIFCPKCYDTRYAPSCAKCKKKITGEVMHALKMTWHVQCFTCAACKTPIRNRAFYMEEGQPYCERDYEKMFGTKCRGCDFKIDAGDRFLEALGFSWHDTCFVCAICQTNLEGKTFYSKKDKPLCKSHAFSHV

SEQ ID NO: 63 - protein, 583 aa, Gallus gallus
MPKPINVRVTTMDAELEFAIQPNTTGKQLFDQVVKTVGLREVWFFGLQYVDSKGYSTWLKLNKKVTQQDVRKENPLQFKFRAKFFPEDVSEELIQEITQRLFFLQVKEAILNDEIYCPPETAVLLASYAVQSKYGDYNKEIHKLGYLANDRLLPQRVLEQHKLTKEQWEERIQNWHEEHRGMLREDSMMEYLKIAQDLEMYGVNYFEIKNKKGTELWLGVDALGLNIYEHDDKLTPKIGFPWSEIRNISFNDKKFVIKPIDKKAPDFVFYAPRLRINKRILALCMGNHELYMRRRKPDTIEVQQMKAQAREEKHQKQLERAQLENEKKKREIAEKEKERIEREKEELMERLRQIEEQTMKAQKELEEQTRRALELDQERKRAKEEAERLEKERRAAEEAKAALAKQAADQMKNQEQLAAELAEFTAKIALLEEAKKKKEEEASEWQHKAFAAQEDLEKTKEELKSVMSAPPPPPPPPVIPPTENEHDEHDENNAEASAELSSDGVMNHRSEEERVTETQKNERVKKQLQALSSELAQARDETKKTQNDVLHAENVKAGRDKYKTLRQIRQGNTKQRIDEFEAM

SEQ ID NO: 64 - protein, 6644 aa, Gallus gallus
MEEEEYEEVVEYYIEETIVEEGEPYEVVTEITDSTSTEFTGPTTITRTIEYEKTSGEGAATPVRKKTIRTKMDTSKFLTPYLQHSNKMKDLFSENKYKEKFNKERGKPYASTIDTPEIRRIKKVQEQLSEVKYRMAGEAARTICHVDEKAWDIEHAKKVSQQVSKVLYKQNWEENKDKYLLPPDAPELVNAIKNTAMFSKKLYTEDWEGDKTLFYPYNDSPELRRVAQAQKALSDIVYKKGHDERKSKYTSLPDPPDVEQAKKVTRQLSDIIYHDDYKNKIKGKWSQTPCYDVVIAKMNAENLSMKKYQEDFENVKDQIYFMQTETPEYEANKRVSDNVSKIKYRADYEKNKAIADYNVLPATENPLLRQLKTAGDVLSDKLYKEAYERSKGTSMNYCETPKFQTDNALKNFSDVKYKDAYQKNILGHYLGSFEDPHQIHCMKVEAMKSDKNYKADYEEEKTKCYFPQTITQEYEAIKKLEQCKDHTYKKHPDQIKFTPVTDSPVQKQAEINSKQLSDKLYRSSGEEVKHKYTLPPDVPQFIQARYNAANVSDAYYKQDYHDLIAKGNNVSLDAIPITRAKASRNIASDYKYKEAYEKAKGQQVGFKSLQDDPKLVHYMHVAKIQSDREYKKDYEKSKTNYHTPPDTFSIQAAKKSQDVASTAHYKNLIHHYTYLPDAMDVELAKNMMQIQSDNVYKQDYNSWFKGIGWSPLGSLDVEKAKKAGDALNEKKYRQHPDTIKFTSVPDSMTMVLAQHNTKQLSDVAYKQEGEKVKHKYKLDPDVPQFIQARVNAFNLSDANYKADWKKTIAKGYDLKPDAIPIIAAKASRNIASDYKYKESYEKDKGRQVGYRSLQDDPKLVHYMHVAKMQSDREYKKDYEVTKTKYHTPLDMFSVTAAKKAQEAVTNTGYKQLIHHYTLLPDSVNLELSRNMMQLQSDNMYKADFNNWLRGVGWLPIQSLEVEKAKKASEILSEKKYRQHPDKLKYSIPLDAMEQVLAKQNAKTMNKRLYTDKWNKEKTSIHVMPDTPEILQSRVNQITMSNKLYKAGWEEDKKKGYDMRPDAIPIKAAKTSQDIASDYKYKLAHEKAKGKHIGFRSLEDDPKLVHFMQVAKMQSDREYKKDYEKAKTNFHTPVDMLSVVAAKKAQEVATNANYKNLIHVYNVLPDAMSLELAKNMMQIQSNNQYRAEYDESMKGVGWMPLGSLEAEKNKKAMEILSEKKYRQHPDKLKYSVPVDSMNMALALHNAKIMDEHQYKQAWEEDKKKVHMTPDIPQFALAKANAFNISDKMYRHSFEEARKKGYDLRSDAIPIKAAKASRDIASDYKYKLGYEQDKGKLVGFRSLQDDPKLVHYMQVAKMQSDREYKKAYETSKTHYQTPSDALSIMAAKEAQDRVTNANYKRLIHHYMLLPDAMSFELYRNMNQIQSNNEYKQDYNEWFKGIGWSPAGSLDVEKSKKATEIASDQKYRQHPSIFPFTKQIDAMDMVLAKHNADIMNKHAYTQAWEKDKTQVHVMPDTPDILQAKQNKANYSQKQYKLDWQEMIKKGYDLTPEAISVKAAKASRDIASDYKYKEGYRKQQGHHIGFRSLQDDPKMMWSMQVAKMQSEREYKKDFEKWKTKFNMPVDMLGFLLAKKCQELVSDIDYKHMLHRWTCLPDQNDVTQAKRVYELQSDNLYKSDLQWLRGIGWSPLGSLESEKNKKASEILSEKKYRQHPDTIKFTSIPDAMNIILAKSNAKNRSDILYREAWDKDKTQVHIMPDTPEILLAKSNLINTSDKHYKLGYEELRRKGYDLPPDAIPLKSAKASRDIASEYQYKTAYRKQLGHHVGARNIEDDPKMMWSMHVAKIQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDNFYKSDLEWMRGIGWVPIGSLDIEKAKRAGQILSDKVYRQPPDTIKFTSVTDSLEMTLAKHNAEMMNKRLYTEAWDKDKTQIHIMPDTPEITLAKQNMHNYSEKLYKQAMEEAKKKGYDLRSDAIPIQAAKASRQIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDNFYKSDLEWMRGIGWVPIGSLDVEKAKRAGQILSDKVYRQPPDTIKFTSVTDSLEMTLAKHNAETMNKRLYTEAWNKDKTTIHVMPDTPEILLAKQNQAHYSQKMYKLALEESKKKGHDLRFDAIPIQAAKASREIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDAVYKSDLEWLKGIGWVPIGSLDVEKAKKAGEILSDRKYRQPADQIKFTSVTDSLAMMLAKHNAEIMNKRLYTEAWDADKTSIHVMPDTPTILLAKANAANVSHKHYVQAWEDAKKKGYDMRADAIPIRSAKASRDIASDYKYKEAHEKQKGHYIGCRTAKEDPKLSWAARAMLLQNDRIYRKAYNDSKAHIHMPVDAMSLQAAKECQTLVSDVDYRHYLHQWTCLPDQNDVMHARKAYDLQSDNVYKSDLEWLRGIGWLTEGSVDVIKAKKAQEILSDRLYRTQPDKMKFTSITDTPDVVQAKINAMQLSNHLYREVWDKDKTQISIPSDTPELLQSKLNALNISNKHYQKAWDEAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCLPDQNDVMHARKAYDLQSDNVYKSDLEWLRGIGWLTEGSVDVVKAKKAQEILSDRLYRTQPDKMKFTSVTDAPDVVQAKINAMQLSNRLYREAWDKDKTQISIPSDTPEMLQSKVNALNISNKHYQKAWDEAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCLPDHNDVVHARKAYDLQSDAVYKSDLEWLRGIGWLPNDSPGVQRVKHAQDLLSDKVYRTPIDSVKYTSVVDSPDILLAKMNAEQLSIPKYKEAWEKDKTMIHIMPDTPEITLARSNAHNYSQKLYKEAWDEVKMSYDLRADAIPIKAAKASREIASDYKYKLDHEKQKGHYVGVPNAKADTKIRFALGIGKVQSELEYKKHFAKWKTQCHLPVDMLSIQSAKHGQSLVSDVDYRHYLHQWICLPDQNDVIHARKAYDLQSDAVYKSDLEWLRGIGWLPNDSLGINHVKHAGDLLNERKYRTKAETLHFTPVADRVDYVTAKKSGEILSDIKYHKDWNEVKSNYTLTDTPQLDMAREAARILNQSLYKESWEKEKATGYLLPPDTVQIRHAKHSNDVQSELKYKADYVKQRGHYVGVASMRDDPKLVWFEHAGEIQNDRLYKSNYHKTKSKIHIPADIMSVVAAKECQALVSDVDYRHYLHQWTCHPDQNDCIQARKAYDLQSDNIYKSDLEWLRGCGWIPLGSVEHKKVKHAQELINKRAYTKDAIENFSKYTSVVDTPDIVLAKINSVNQSDLKYKETFNLEKGQYIGSDDTPELNHARDMSLLYSDKLYKRDWEVCKPIGYTLDAKYIPLVGAKHANYVNSELKYKEIYEKLKGHYLAGKDIGDFPSVVHSLAFQKIRSALAYRKNYEDTKTRVHIPSDMMNHVLAKKCQYILSDLEYRTYLHHWNCSPEEHDVIQARRAQEILSDVVYKDDLNWLKGIGCYVWDTPQILHAKKSYDLQSQIKYTAAGKENFQNFGVVTDTPVYVTAVQSGINASDVKYKEDYHKTKDKYTTVTETADSERVQNLKHLFSNNLYKEAWDRVKATSYIMPSDAVSLARAKELKHNASIVKYREEYDKFKALYTLPRSVEDDPNTARCLRVGKLNIDRLYKETYEKNKAKVHIIPDMVDIIAAKDAQKKISEIDYRTHLHDWICLPDLQINAHVRKVADQISDVVYKDDLNWLKGIGCFVWDTPEILHAKHAYDLRSDIKYKSDADKMKSKYTVVMDTPVYVQNILSGLNASEVIYRGDYLKKVRGKMIPTDKTVDLQRAHHANKIQSENLYRWAGLKALPTGYSLPKDTPGFQHAKHVQHIGSDLKYKEAYEHMKAKGYTLGPNDVGFENVKKVNQVINERLYRATYHKNKDKIHTTPDTPEIRQVRATQEAVSDLIYKSDFFKLQGHLISLPFTPQVLHCRYVGDITSDNKYKEDLKWLQGLGCFLYDTPDMVRARQLRKLWSNYVYTDSAKKMRDKYSVVLDTPGYRTVQELKTHLSDLVYRAAGKELKTKYTSVLNTPDFLRAKEGQRIQSQYLYVALATKERPHHHAGNQTPAFTHARHVKDMVSETKYKIQYEKMKDKYTPVLDTPILIRAKRAYLNASDLRYKETFENTKGKYHTVKDALDIVYHRKVTDDISSVKYKENYMSQLGIWRSIPDRPEHFHHRAVTDAISDVKYKEDLSWLRGIGCYAWDTPDFALAEKNKVLYSGHKYKETFEKTKSHFKYVADSPINRHFKHATQLLDANSYKSLAKMLLKQGCNEILRPDILTALYNSYLWSQVEYKKDYEKKKDKYTTVVDTPENIRTAKVNKQISDIIYKLEYNKAKPKGYTTIHDTPMLLHVRKVKDRISDLKYKELYERNKSHCNVVADSVHIKTPRHAYKLNSNLDYKKKYEAAKAHWHWIADRPDFVQAAKSSLQQSDYEYKLDREYLKGCKLSVTDDKNTVLALNNAILASDIKYKEKHNKARGTCLVVPDTPQILLAKNVSSLVSENKYKEHSKKQLPRGSYTTLPETRDTAHVKEVTKNVSDTNYKKKFVKEKGKSNYSIMLEPPEVKHAMEVAKKQSTVEYKKDAKSKLHYTPIADRPDIKKATQAAKLISDIEYKKRGEAGLGVTMLGRPDIELAKEVSKLTSQVKYKENFSKEKGKKPKYDLKEAKIYKTMKDAHNIASEVKYKADLKKLHKPVTDMSESLIMNHVLNTSQLASAVKYKEKYEKEKGKPMLDFETPTYLTAKESQLMQSEKEYRKDLEEGVKGKGLSVLEETPDMLRAKNATQILNEKEYKKALELEIKGKGLSELALETPDFVRAKNATDIASQIKYKQLAEMEKANYTSVVDTPEIIHAQQVKNLSSQKKYKEEAEKTMPYYVPVADTPEMQRVRENQKNFSTLQYQWDLQNSKGKVTVVQDTPEMLRVKENQKNFSSIKYKESIGKGTPIPDLPEVKRVKETQKHISSVLYKEHLAKGTPTPMTPEMERAKRNQENISSVLYSDSFRKQVQGKAAYVLDTPEMRRVRETQKHISTVKYHEDFEKNKGTFTPVVTDPITERVKKNMHDFSDISYRGIQRRVVEMEQKRVDQDQENLTGLRVWRTNPGSVFDYDPAEDNIQSRSLHMISVQAQRRSREHSRSASALSISGGDEKSEPSEGVNQHLSYYSSGGFFTTTATVGYKHAKTIELPQQRSASVATQQTTVSSVPSHPSTAGKTYRAMYDYTAADADEVSFKDGDTIVNVQAIDEGWMYGTVQRTGKTGMLPANYVEAV

SEQ ID NO: 65 - protein, 6754 aa, Gallus gallus
MEEEEYEEVVEYYIEETIVEEGEPYEVVTEITDSTSTEFTGPTTITRTIEYEKTSGEGAATPVRKKTIRTKMDTSKFLTPYLQHSNKMKDLFSENKYKEKFNKERGKPYASTIDTPEIRRIKKVQEQLSEVKYRMAGEAARTICHVDEKAWDIEHAKKVSQQVSKVLYKQNWEENKDKYLLPPDAPELVNAIKNTAMFSKKLYTEDWEGDKTLFYPYNDSPELRRVAQAQKALSDIVYKKGHDERKSKYTSLPDPPDVEQAKKVTRQLSDIIYHDDYKNKIKGKWSQTPCYDVVIAKMNAENLSMKKYQEDFENVKDQIYFMQTETPEYEANKRVSDNVSKIKYRADYEKNKAIADYNVLPATENPLLRQLKTAGDVLSDKLYKEAYERSKGTSMNYCETPKFQTDNALKNFSDVKYKDAYQKNILGHYLGSFEDPHQIHCMKVEAMKSDKNYKADYEEEKTKCYFPQTITQEYEAIKKLEQCKDHTYKKHPDQIKFTPVTDSPVQKQAEINSKQLSDKLYRSSGEEVKHKYTLPPDVPQFIQARYNAANVSDAYYKQDYHDLIAKGNNVSLDAIPITRAKASRNIASDYKYKEAYEKAKGQQVGFKSLQDDPKLVHYMHVAKIQSDREYKKDYEKSKTNYHTPPDTFSIQAAKKSQDVASTAHYKNLIHHYTYLPDAMDVELAKNMMQIQSDNVYKQDYNSWFKGIGWSPLGSLDVEKAKKAGDALNEKKYRQHPDTIKFTSVPDSMTMVLAQHNTKQLSDVAYKQEGEKVKHKYKLDPDVPQFIQARVNAFNLSDANYKADWKKTIAKGYDLKPDAIPIIAAKASRNIASDYKYKESYEKDKGRQVGYRSLQDDPKLVHYMHVAKMQSDREYKKDYEVTKTKYHTPLDMFSVTAAKKAQEAVTNTGYKQLIHHYTLLPDSVNLELSRNMMQLQSDNMYKADFNNWLRGVGWLPIQSLEVEKAKKASEILSEKKYRQHPDKLKYSIPLDAMEQVLAKQNAKTMNKRLYTDKWNKEKTSIHVMPDTPEILQSRVNQITMSNKLYKAGWEEDKKKGYDMRPDAIPIKAAKTSQDIASDYKYKLAHEKAKGKHIGFRSLEDDPKLVHFMQVAKMQSDREYKKDYEKAKTNFHTPVDMLSVVAAKKAQEVATNANYKNLIHVYNVLPDAMSLELAKNMMQIQSNNQYRAEYDESMKGVGWMPLGSLEAEKNKKAMEILSEKKYRQHPDKLKYSVPVDSMNMALALHNAKIMDEHQYKQAWEEDKKKVHMTPDIPQFALAKANAFNISDKMYRHSFEEARKKGYDLRSDAIPIKAAKASRDIASDYKYKLGYEQDKGKLVGFRSLQDDPKLVHYMQVAKMQSDREYKKAYETSKTHYQTPSDALSIMAAKEAQDRVTNANYKRLIHHYMLLPDAMSFELYRNMNQIQSNNEYKQDYNEWFKGIGWSPAGSLDVEKSKKATEIASDQKYRQHPSIFPFTKQIDAMDMVLAKHNADIMNKHAYTQAWEKDKTQVHVMPDTPDILQAKQNKANYSQKQYKLDWQEMIKKGYDLTPEAISVKAAKASRDIASDYKYKEGYRKQQGHHIGFRSLQDDPKMMWSMQVAKMQSEREYKKDFEKWKTKFNMPVDMLGFLLAKKCQELVSDIDYKHMLHRWTCLPDQNDVTQAKRVYELQSDNLYKSDLQWLRGIGWSPLGSLESEKNKKASEILSEKKYRQHPDTIKFTSIPDAMNIILAKSNAKNRSDILYREAWDKDKTQVHIMPDTPEILLAKSNLINTSDKHYKLGYEELRRKGYDLPPDAIPLKSAKASRDIASEYQYKTAYRKQLGHHVGARNIEDDPKMMWSMHVAKIQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKVQSDREYKKAFEKTKTHFSSPVDMLGIVLAKKCQELVSDVDYKHLLHRWTCLPDQNDVVQARKVYDLQSDNVYKSDLQWLRGIGWSPLGSLDEEKNKRASMILSDKKYRQHPDTIKFTSLPDSMPMVLAKHNSEIMNHRSYIAAWEKDKTSIHIMPDTPGILLAQQNKVNYSEKMYRLAMEEDKKKGYDLRADAIPIKAAKASRDIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDNFYKSDLEWMRGIGWVPIGSLDVEKAKRAGQILSDKVYRQPPDTIKFTSVTDSLEMTLAKHNAETMNKRLYTEAWNKDKTTIHVMPDTPEILLAKQNQAHYSQKMYKLALEESKKKGHDLRFDAIPIQAAKASREIASDYKYKEGYRKQLGHHIGARNIEDDPKMMWSMHVAKIQSDREYKKAFEKTKTHFSSPVDMLGIVLAKKCQELVSDVDYRHYLHQWICLPDQNDVIHARKAYDLQSDAVYKSDLEWLKGIGWVPIGSLDVEKAKKAGEILSDRKYRQPADQIKFTSVTDSLAMMLAKHNAEIMNKRLYTEAWDADKTSIHVMPDTPTILLAKANAANVSHKHYVQAWEDAKKKGYDMRADAIPIRSAKASRDIASDYKYKEAHEKQKGHYIGCRTAKEDPKLSWAARAMLLQNDRIYRKAYNDSKAHIHMPVDAMSLQAAKECQTLVSDVDYRHYLHQWTCLPDQNDVMHARKAYDLQSDNVYKSDLEWLRGIGWLTEGSVDVIKAKKAQEILSDRLYRTQPDKMKFTSITDTPDVVQAKINAMQLSNHLYREVWDKDKTQISIPSDTPELLQSKLNALNISNKHYQKAWDEAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCLPDQNDVMHARKAYDLQSDNVYKSDLEWLRGIGWLTEGSVDVVKAKKAQEILSDRLYRTQPDKMKFTSVTDAPDVVQAKINAMQLSNRLYREAWDKDKTQISIPSDTPEMLQSKVNALNISNKHYQKAWDEAKAKNYDLRADAIPIKHAKASRDIASEYKYKEAHEKQKGHYIGCRTAKEDPKLSWAARAMLLQNDRIYRKAYNDSKAHIHMPVDAMSLQAAKECQTLVSDVDYRHYLHQWTCLPDHNDVVHARKAYDLQSDAVYKSDLEWLRGIGWLPNDSPGVQRVKHAQDLLSDKVYRTPIDSVKYTSVVDSPDILLAKMNAEQLSIPKYKEAWEKDKTMIHIMPDTPEITLARSNAHNYSQKLYKEAWDEVKMSYDLRADAIPIKAAKASREIASDYKYKLDHEKQKGHYVGVPNAKADTKIRFALGIGKVQSELEYKKHFAKWKTQCHLPVDMLSIQSAKHGQSLVSDVDYRHYLHQWICLPDQNDVIHARKAYDLQSDAVYKSDLEWLRGIGWLPNDSLGINHVKHAGDLLNERKYRTKAETLHFTPVADRVDYVTAKKSGEILSDIKYHKDWNEVKSNYTLTDTPQLDMAREAARILNQSLYKESWEKEKATGYLLPPDTVQIRHAKHSNDVQSELKYKADYVKQRGHYVGVASMRDDPKLVWFEHAGEIQNDRLYKSNYHKTKSKIHIPADIMSVVAAKECQALVSDVDYRHYLHQWTCHPDQNDCIQARKAYDLQSDNIYKSDLEWLRGCGWIPLGSVEHKKVKHAQELINKRAYTKDAIENFSKYTSVVDTPDIVLAKINSVNQSDLKYKETFNLEKGQYIGSDDTPELNHARDMSLLYSDKLYKRDWEVCKPIGYTLDAKYIPLVGAKHANYVNSELKYKEIYEKLKGHYLAGKDIGDFPSVVHSLAFQKIRSALAYRKNYEDTKTRVHIPSDMMNHVLAKKCQYILSDLEYRTYLHHWNCSPEEHDVIQARRAQEILSDVVYKDDLNWLKGIGCYVWDTPQILHAKKSYDLQSQIKYTAAGKENFQNFGVVTDTPVYVTAVQSGINASDVKYKEDYHKTKDKYTTVTETADSERVQNLKHLFSNNLYKEAWDRVKATSYIMPSDAVSLARAKELKHNASIVKYREEYDKFKALYTLPRSVEDDPNTARCLRVGKLNIDRLYKETYEKNKAKVHIIPDMVDIIAAKDAQKKISEIDYRTHLHDWICLPDLQINAHVRKVADQISDVVYKDDLNWLKGIGCFVWDTPEILHAKHAYDLRSDIKYKSDADKMKSKYTVVMDTPVYVQNILSGLNASEVIYRGDYLKKVRGKMIPTDKTVDLQRAHHANKIQSENLYRWAGLKALPTGYSLPKDTPGFQHAKHVQHIGSDLKYKEAYEHMKAKGYTLGPNDVGFENVKKVNQVINERLYRATYHKNKDKIHTTPDTPEIRQVRATQEAVSDLIYKSDFFKLQGHLISLPFTPQVLHCRYVGDITSDNKYKEDLKWLQGLGCFLYDTPDMVRARQLRKLWSNYVYTDSAKKMRDKYSVVLDTPGYRTVQELKTHLSDLVYRAAGKELKTKYTSVLNTPDFLRAKEGQRIQSQYLYVALATKERPHHHAGNQTPAFTHARHVKDMVSETKYKIQYEKMKDKYTPVLDTPILIRAKRAYLNASDLRYKETFENTKGKYHTVKDALDIVYHRKVTDDISSVKYKENYMSQLGIWRSIPDRPEHFHHRAVTDAISDVKYKEDLSWLRGIGCYAWDTPDFALAEKNKVLYSGHKYKETFEKTKSHFKYVADSPINRHFKHATQLLDANSYKSLAKMLLKQGCNEILRPDILTALYNSYLWSQVEYKKDYEKKKDKYTTVVDTPENIRTAKVNKQISDIIYKLEYNKAKPKGYTTIHDTPMLLHVRKVKDRISDLKYKELYERNKSHCNVVADSVHIKTPRHAYKLNSNLDYKKKYEAAKAHWHWIADRPDFVQAAKSSLQQSDYEYKLDREYLKGCKLSVTDDKNTVLALNNAILASDIKYKEKHNKARGTCLVVPDTPQILLAKNVSSLVSENKYKEHSKKQLPRGSYTTLPETRDTAHVKEVTKNVSDTNYKKKFVKEKGKSNYSIMLEPPEVKHAMEVAKKQSTVEYKKDAKSKLHYTPIADRPDIKKATQAAKLISDIEYKKRGEAGLGVTMLGRPDIELAKEVSKLTSQAEYLKRHMRGHGAASYDTPWMRTFKKANMLSSHVKYKENFSKEKGKKPKYDLKEAKIYKTMKDAHNIASEVKYKADLKKLHKPVTDMSESLIMNHVLNTSQLASAYQYKKQYEKSKGHYHMVPDNLEQVHLREASELQSHVKYKEKYEKEKGKPMLDFETPTYLTAKESQLMQSEKEYKRDFEESIKGRNLTGLEITPSLLHVKYATKIASEKEYRKDLEEGVKGKGLSVLEETPDMLRAKNATQILNEKEYKKALELEIKGKGLSELALETPDFVRAKNATDIASQIKYKQLAEMEKANYTSVVDTPEIIHAQQVKNLSSQKKYKEEAEKTMPYYVPVADTPEMQRVRENQKNFSTLQYQWDLQNSKGKVTVVQDTPEMLRVKENQKNFSSILYKEDIGTGTTIEKTPEMQRVKRTQDAISSIKYKESIGKGTPIPDLPEVKRVKETQKHISSVLYKEDLGTGIPTPVTPEIERVKRNQEHISSVLYKEELGTGTPIPVTPEVERVKRNQEHISSVLYKESVGTGTPTPITPEMERVKRNQEICSSVLYKENIGKATPTPVTPEMERVKRNQEIISSVLYKENVGKVTPTPITPEMERVKRNQEIISSVLYKESLGRATPTPITPEMERVKRNQEQISSVVYKEGLGKATPTPVTPEMERVRRNQEQISSVLYKEHLAKGTPTPMTPEMERAKRNQENISSVLYSDSFRKQVQGKAAYVLDTPEMRRVRETQKHISTVKYHEDFEKNKGTFTPVVTDPITERVKKNMHDFSDISYRGIQRRVVEMEQKRVDQDQENLTGLRVWRTNPGSVFDYDPAEDNIQSRSLHMISVQAQRRSREHSRSASALSISGGDEKSEPSEGVNQHLSYYSSGGFFTTTATVGYKHAKTIELPQQRSASVATQQTTVSSVPSHPSTAGKTYRAMYDYTAADADEVSFKDGDTIVNVQAIDEGWMYGTVQRTGKTGMLPANYVEAV

SEQ ID NO: 66 - protein, 6909 aa, Bos taurus
MADDEEYEEVVEYYTEETVYEEVPGETRTRFYETTTTRTSDYEQSETSRPALAQPVPEKPVERKRVIRKKVDPSKFMTPYIAHSQKMQNLFSTNKYKENYEKAKGKPYAITTDTPELRRIKKVQDQLSEVKYRVDGDVAKTICHVDEKAKDIEHAKKVSQQVSKVLYKQNWEDTKDKYLLPPDAPELVQAIKNTAMFSKKLYTEDWEADKTMFYPYNDSPELRRVAQAQKALSDIAYKKGLAEQQTQFTSLPDPPDIEFAKKVTNQVSKQKYKEDYENKVKGKWSETPCFEIATARMNSNNISTKKYQEDFEHMKDQIYFMQTETPEYKVNKQAGVAASKVKYKQDYEKNKGKADYNVLPASENPLLRQLKAAGDALSDKLYKENYEKTKAKSINYCETPKFKLDTVLHNFSSDTKYKDSYLKDILGHYVGSYEDPYHTHCMRVSAQNSDKNYKAEYEEDRGKGFFPQTITQEYEAIKKLDQCKDHTYKVHPDKTKFTQVTDSPVLMQAQVNSKQLSDLNYKAKHENEKFKCHIPPDTPAFIQHKLNAYNLSDNIYKHDWEKTKAKKFDIKVDAIPLLAAKANTKIASDVMYKKDYEKSKGKMIGALSINDDPKMLHSLKTAKNQSDRLYKENYEKTKAKSMNYCETPKYQLDTLLKNFSEAKYKDSYVQNVLGHYIGSFEDPYQVHCLKISAQNSDKNYKAEYEEDKGKCYFPQTITQEYEAIKKLDQCKDHTYKVHPDKTKFTAVTDTPVLLQAQLNTKQLSDLNYKAKHEGEKFKCHIPADAPQFIQHRVNAYNLSDNIYKHDWEKTKAKKFDIKVDAIPLLAAKANTKIASDVMYKKDYEKSKGKMIGALSINDDPKMLHSLKTAKNQSDHEYRKDYEKSKTIYTAPLDMLPLTHAKKSQAIASDVDYKHLLHNYSYPPDSVNVDLAKKAYALQSDVEYKADYNSWMKGCGWMPFGSLEMEKAKRASDILNEKKYRQHPDTLKFTSIEDAPIIVQSKINQAQRSDVAYKAKGEEVIHKYSLPADLPQFIQAKVNAYNISENLYKADLKDLSKKGYDLRIDAIPIKAAKAARQAASDVQYKKDYEKAKGKMVGFQSLQDDPKLVHYMNVAKIQSDREYKKAYEKTKTRHNTPHDMVNIVAAKKAQDVASNVNYKHSLHHYTYLPDAMDLELSKNMMHIQSDNVYKEDYNNWMKGIGWIPIGSLEVEKVKKAGDALNEKKYRQHPDTLKFTSIVDSPVMVQAKQNTQQVSDILYKAKGEDVKHKYTMSPDLPQFLQAKCNAYNLSDVCYKRDWHDLIAKGTNVLGDAIPITAAKASRNIASDYKYKEAYEKAKGKQVGFRSLQDDPKLVHYMNVAKLQSDREYKKNYENTKTSYHTPGDMVSITAAKMAQDVATNVNYKQPIHHYTYLPDALSLEHIRNVNQIQSDNVYKDEYNHFFKGMGWIPIGSLEVEKVKKAGDALNEKKYRQHPDTIKFTSVPDSMGMVLAQHNTKQLSDLNYKVEGEKVKHKYTMDPDVPQFIQAKVNAYNMSDSHYKADWKKTLAKGYDLRPDAIPIVAAKSSRNIASDFKYKEAYEKTKGKQIGFRSLQDDPKLVHFMNVAKMQSDREYKKGYEASKTKYHTPLDMLSVTAAKKSQEVATNTNYKQPFHHYTLLPDALNVEHSRNAMQIQSDNLYKSDFTNWMKGIGWLPLESLEVEKAKKAGEILSEKKYRQHPEKLKFTYAMDTMEQALNKSNKLIMDKRLYTEKWNKDKTTIHVMPDTPDILLSRVNQITMSDKLYKAGWEEEKKKGYDLRPDAISIKAARASRDIASDYKYKQAYEQAKGKQIGFRSLEDDPKLVHFMQVAKMQSDREYKKAYEKSKTSFQTPVDMLSVVAAKKSQEVATNANYRNVIHTYNMLPDAMSLELAKNMMQIQSDNQYKADYADFMKGIGWLPLGSLEAEKNKKAMEIISEKKYRQHPDTLKYSTLMDSMNMVLAKNNAKIMNEHLYKQAWEADKTKVHIMPDIPQIILAKANAINISDKLYKLSLEEAKKKGYDLRTDAIPIKAAKASRDIASDYKYKHSYEKERGKMVGFRSLEDDPKLVHSMQVAKMQSDREYKKNYEKTKTSYHTPADMLSVTAAKDAQANITNTNYKHLIHKYILLPDAMNIQLSKNMNRIQSDNEYKQDYNEWYKGLGWSPAGSLEVEKAKKATEYASDQKYRQHPSNFQFTKLNDSMDMVLAKQNAHTMNKYLYTVDWNKDKTKIHVMPDTPDILQAKQNQTMYSQVKYKMGWPSGLERVVNLPSVLIKIFKSKQNKDLIWRFKYKQGYRKQIGHHIGFRSLQDDPKLVLSMNVAKMQSEREYKKDFEKWKTKFSSPVDMLGVVLAKKCQALVSDVDYKNYLHGWTCLPDQNDVIHAKKAYDLQSENLYKSDLEWLKGIGWSPLGSLEAEKNKRASEIISEKKYRQPPDRNKFTSIPDAMDIVLAKTNAKNRSDILYREAWDKDKTQIHIMPDTPDIILAKANLINTSDKFYRMGYEELRKKGYDLPVDAIPIKAAKASREIASEYKYKEGFRMQLGHHIGARNIKDDPKMMWSMHVAKIQSDREYKKDFEKWKTKFSSPVDMLGVVLAKKCQTLVSDIDYKNYLHQWTCLPDQSDVIHARRAYDLQSDNLYKSDLQWLRGIGWLPSGSLEDEKNKRASQILSDHVYRQHPDQFKFSSLMDSIPMVLAKNNAITMNHRLYTEAWDKDKTTVHIMPDTPEVLLAKQNQINYSEKLYKLGLDEAKRKGYDMRIDAIPIRAAKASRDIASEFKYKEGYRRQLGHHIGARAIHDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLRGIGWVPIGSLDVEKSKRASEILSDKLYRQPPDKFKFTSVTDSLEQVLAKNNAINMNKRLYTEAWDKDKTQVHIMPDTPEITLARTNKVNYSENLYKLAHEEAKKKGYDLRSDAIPIIAAGLEKDHISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLRGIGWVPIGSVDVVKCKRAAEILSDNLYRQPPDTLKFTSVPDSLEQVLAKNNAINMNKRLYTEAWDKDKTQIHIMPDTPEIMLARQNKLNYSETLYKLANEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARRAYDLQSDNIYKSDLQWLRGIGWVPIGSVDVVKCKRAAEILSDNLYRQRPDTLKFTSVPDSLEQVLAKNNAINMNKRLYTEAWDKDKTQIHMMPDTPEITLARQNKINYSENLYRQAMEEAKKEGYDLRSDAIPIVAAKASREIASDYKYKEAYRKQLGHHIGARAIHDDPKMMWSVHVAKMQSDREYKKDFEKYKTRFSSPVDMLGIVLAKKCQTLVSDVDYKHPLHEWTCLPDQNDIIHARKAYDLQSDNLYKSDLEWLKGIGWVPIGSVEVLKAKRAGEILSDNIYRQRPDTLKFTSVTDSPEQVLAKNNAINMNKRLYTEAWDNDKKTIHVMPDTPEIMLAKLNRINYSDKLYKLALEESRKEGYDLRLDAIPIQAAKASREIASDYKYKEGYRKQLGHHIGARNIKDDPKMMWSIHAGKLQSDLEYKKDFEKWKTKFSSPVDMMGLVQAKKCQILVSDIDYKNLLHEWTCLPDQNDIIQARKAYDLQSDAIYKADLEWLRGIGWVPIDSVGVEHAKRAGEILSERKYRQPAVQLKFTSITDTPEIVLAKNNALNVSKHLYTEAWDADKTSIHVMPDTPEILLAKSNSANISHKLYTKGWDESKMKDYDLRADAISIKSAKASRDIASDYKYKEAYEKHKGHHIGARSVEDDPRIMCAMNAGRIQSEREYKKEFQKWKTKFSSPVDMLGILLAKKCQTLVSDIDYRNYLHHWTCLPDQNDIIQARKAYDLQSDAIYKADLEWLRGIGWMPEGSPEVLRVKNAQHIFRDSVYRTPVVKLKYTSIVDTPEVVLAKSNAENISIPKYREVWDKDKTSIHIMPDTPEINLARTNALNVSNKLYREGWDEMKMSCDVRLDAIPIQAAKASREIASDYKYKLDHEKQKGHYVGTRTARDDNKIRWALIAGKIQNEREYRLHWAKWKSKFQSPPDMLSIEHSKNSQTLVSDIDYRHYLHQWTCMPDQNDVIQARKAYDLQSDAIYKADLEWLRGIGWMPADSVSVNHAKHMADIFNEKKYRTKIETLSFTPVDDRVDYVTAKHSGEILNDIKYRKDWNDTKSKYTLTETPQLHTAQEAARILDQYLYKESWEKQKATGYILPPDAVPFVHAHHSGDVQSELKYKAEHVKQKGHYVGVPTMRDDPKLVWFEHAGQIQNDRLYKENYHKTKAKIHIPPDMVSVLAAKEGQALASDIDYRNYLHQWICHPDQNDVIQARKAYDLQSDNIYKADLEWLRGIGWIPLDSVDHVRVTRNQEMMNQIKYKKDALANYPNFTSVVDPPEIVLAKINSVNQSDVKYKETFNKLIKGKYIFSPDTPYITHSKDMEKLYSTILYKRAWDGTKAYGYTLDERYIPIVGAKHADFVNSELKYKETYEKLKGHYLAGKEISEFPNVVHCLDFQKMRSLLNYRRHYEDTKANVHIPQDMMNHVLAKRCQYILSDLEYRHYFHQWTSLPEEPNVVRVRHAQEILSDNVYKDDLNWLKGIGCYVWDTPQILHAKKSYDLQSQILYTAAGKENLKNYNLVTDTPLYVTALQSGINASEVKYKENYHQTKDKYTTVLETVDYDRIKNLKDLFSSNLYKEAWDKVKATSYILPPNTVSLTHAKNQKYMASHIKYREEYEKFKALYTLPRSVEDDPNTARCLRVGKFNIDRLYRSVYEKNKMKIHIVPDMVEMVTAKDSQKKVSEIDYRLHLHEWICHPDLQVNSHVRKVTDQISDIVYKDDLTWLKGIGCYVWDTPEILHAKHAYDLRNDIKYKAHVLKTRNNYKLVTDTPVYVQAVKSGKQLSDAVYHYDYVHSIRGRVAPTTKTVDLDRALHAYKLQSENLYRKAGLHALPTGYRLPVDTPHFKHTKDTRYMSSYFKYKEVYEHMKAYGYTLGPNDVPFVNVRRVNNITSERLYRQLYHKLKDKIHTTPDTPEIRQVKKTQEAVSELIYKSDFFKMQGHMISLPYTPQVLHCRYVGDITSDIKYKEDLQVLRGMGCFLYDTPDMVRSRHLRKLWSHYLYTDKARKMRDKYKVVLDTPEYRKVQELKTHLSELVYRAAGRKQKSIFTSVPDTPDLTRAKRGQKLQSQYLYVELATKERPHHHAGNQTTALKHARHVKDMVSENKYKIQYEKMKDKYTPVPDTPILIRAKRAYWNASDLRYKETFQKTKGKYHTVKDALDIVYHRTVTDHISKIKYKENYMSQLGIWRSIPDRPEHFHHRAVTDAVSDVKYKQDLTWLKGIGCYAYDTPDFTLAEKNKTLYSKYKYKEVFERTKSNFKYVADCPINRHFKFATQLMNEKKYRADYEQRKDKYHLVVDEPRHLLAKIAGDQISQIKYRKNYEETKDKFTSIVDTPEHLRTTKVNKQISDILYKLEYNKAKPRGYTTIHDTPMLLHVRKVKDEVSDLKYKEVYQRTKSNCTIEPDAVHIKAAKDAYKVNTNLDYKKKYEATKAYWKWTPDRPDFIQAAKSTLQQSDFEYKLDREYLKGCKLSVTDDKDMVLALKNSIIESDLKYKEKHVKERGSCHAVPDTPQILLAKTVSSLVSENKYKSYVKKHLAQGSYTTLPETRDTIHVKEVTKNVSDTNYKKKFVKEKGKSNYSIMLEPPDVKHAMDVAKKQSNVAYKKDAKENLHYTTVADRPDIKKATQAAKQASEVEYRAKHRKEGSHGLSMLGRPDIEMAKKAAKLSSQVQYRENFNKEKGKTPKYNPKDSQLYKVMKDANTLASEVKYKADLKKLHKPVTDMKESLIMNHVLNTSHLASSYQYKKNYEKSKGHYHTIPDNLEQLHLKEATELQSIVKYKEKYEKERGKPMLDFETPTYITAKESQQMQSGKEYRKDYEESIKGRNLTGLEVTPALLHVKYATKIASEKEYRKDLEESIRGKGLSEMEDTPDMLRAKNATQILNEKEYKRDLELEVKGRGLNAMANETPDFLRARNATDIASQIKYKQSAEMEKANFTSVVDTPEIIHAQQVKNLSSQKKYKEDAEKCMSYYETVLDTPEMQRVRENQKNFSLLQYQYDLKNSKGKITVVQDTPEILRVKENQKNFSSVLYKEDVSPGTAIGKTPEMMRVKQTQDHISSVKYKEVIGQGTPIPDLPEVKRVKQTQKHISSVMYKENLGTGIPTPVTPEIERVKRNQENFSSVLYKENLGKGTPTPITPEMERVKRNQENFSSILYKENLSKGTPLPVTPEMERVKRNQENFSSVLYKENVGKGTPTPVTPEMQRVKRNQENISSVLYKENLGKATPTPFTPEMERVKRNQENFSSVLYKENMRKATPTPVTPEMERAKRNQENISSVLYSDSFRKQIQGKAAYVLDTPEMRRVRETQRHISTVKYHEDFEKHKGCFTPVVTDPITERVKKNTQDFSDICYRGIQRKVVEMEQKRNDQDQETITGLRVWRTNPGSVFDYDPAEDNIQSRSLHMINAQAQRRSREQSRSASGLSISGGEEKSEHSEAAHLSTYSDGGVFFSAASTGYKHARTTELPQQRSSSVATQQTTVSSIPSHPSTAGKIFRAMYDYMAADADEVSFKDGDAIVNVQAIDEGWMYGTVQRTGRTGMLPANYVEAI

SEQ ID NO: 67 - protein, 499 aa, Bos taurus
MFNYERPKHFIQSQNPCGSRLQPPGPEISSYSSQTKQSSITIQPRQCTEQRYSASSTVSSHITMSSSAFPASPQQLAGSNPAQRVTATYNQSPASFLSSILPSQPDYSSSKNPSTVDSNYQQPSVGQPINVKSSQNANAKLTPKTPDHEIQGSKEALIQDLERKLKCKDSLLHNGNQRLTYEEKMARRLLGPQNAAAVFQGQNDSEAQDSAQQHNIEHARLQVPTSQVRSRSSSRGDVNDQDAIQEKFYPPRFIQVPENMSIEEGRFCRMDFKVSGLPAPDVSWYLNGRPVQSDDFHKMIVSEKGFHSLIFEVVRASDAGAYACVAKNRAGEATFTVQLDVLAKEHRRAPMFIYKPQSKKVFEGESVKLECQISAVPPPKLFWKRNNEMVQFNTDRISLYHDNSGRVTLLIKDVNKKDAGWYTVSAVNEAGVTTCNTRLDVTARPNQTLPAPKQLRVRPTFSKYLALNGKGLNVKQAFNPEGEFQRLAAQSGLYESEEL

SEQ ID NO: 68 - protein, 513 aa, Gallus gallus
MSVRNLPSVSSMCQPTMFNYERPKHFIQSKNVCQGQQQPPGSSTSTESSRQIKQSSILIQPRNPSGQKFSSSSSLSSSITLSSPSCSAPKESTYPVTPASAQSPASSSSGQRLISMPNQTPAAFLCSVLPSQPDYNSQTPPMEPHYSKPMYKKQASINSMQKTSDQEIRGTKEALIQDLEKKLRCKDNLLQNGNQRLTYEERMARRLLGPENAASVLEAQSEDMQNTQNAENVRLQVPTTHVRSRPSSRGDERGHDSIQEKFFQPRFTQVPEDVIIEEGRFCRLDFKVSGLPTPDVMWYQNGRMVHQDQFHKMIVSEKGFHSFIFEAVKSSDAGTYECVAVNRAGESSFAVKVEVIAKEHHTPPTFIFKPQSKKVFEGDTARLECQISAIPTPRIYWKRNNEMVQYNTDRISLLHDNTGRICLLIHNANKKDAGWYTVSAVNGAGVATCHARLEVATHTNKPVPAPKQLRVRPTFSKYLALNGRGLDVKQAFSPEGEFQRLAEQSGLYESDEL

SEQ ID NO: 69 - protein, 499 aa, Sus scrofa
MFNYERPKHFIQSQNPCGSRLQPPGPETSSYSSQTKQSSIIIQPRQCTEQRFSASSTVSSHITMSSSAFPASPQQPAASNPGQRVTATYNQSPASFLSSILPSQPDYSSSKIPSTMDSNYQQPSVGQPVNAKPSQSLNAKPIPRTPDHEIQGSKEALIQDLERKLKCKDSLLHNGNQRLTYEEKMARRLLGPQNAAAVFQAQNDSAAQDSPQQHNSEHARLQVPTSQVRSRSSSRGDVNDQDAIQEKFYPPRFIQVPENMSVEEGRFCRMDFKVSGLPAPDVSWYLNGRPVQSDDLHKMIVSEKGFHSLIFEVVRASDAGAYACVAKNRAGEATFTVQLDVLAKEHRRAPMFIYKPQSKKVFEGDSVKLECQISAIPPPKLFWKRNNEMVQFNTDRISLYHDNSGRVTLLIKDVNKKDAGWYTVSAVNEAGVITCNTRLDVTARPNQTLPAPKQLRVRPTFSKYLALNGRGLDVKQAFNPEGEFQRLAAQSGLYESEEL

SEQ ID NO: 70 - protein, 349 aa, Sus scrofa
MAHQTGIHATEELKEFFAKARAGSVRLIKVVIEDEQLVLGASRELMGCWDQDYDRAVLPLLDAQQPCYLLYRLDTQNAQGFEWLFLAWSPDNSPVRLKMLYAATRATVKKEFGGGHIKDELFGTVKDDLSFAGYQKHLSSCAAPAPLTSAERELQQIRINEVKTEISVESKHQTLQGLAFPLQPQAQRALQQLRQKMINYIQLKLDLERETIELVHTEPTDVAQLPSRVPRDAARYHFFLYKHTHEGDPLESVVFIYSMPGYKCSIKERMLYSSCKSRLLDSVEQDFQLEIAKKIEIGDGAELTAEFLYDEVHPKQHAFKQAFAKPKGPGGKRGHKRLIRGPGENGDDS

SEQ ID NO: 71 - protein, 3679 aa, Bos taurus
MLWWEEVEDCYEREDVQKKTFTKWINAQFSKFGKQHIENLFSDLQDGRRLLDLLEGLTGQKLPKEKGSTRVHALNNVNKALQVLQKNNVDLVNIGSSDIVDGNHKLTLGLIWNIILHWQVKNVMKNIMAGLQQTNSEKILLSWVRQSTRNYPQVNVINFTTSWSDGLALNALIHSHRPDLFDWNSVVRQQSATQRLEHAFNIAKYQLGIEKLLDPEDVATTYPDKKSILMYVTSLFQVLPQQVSLEAIQEVEMLPRPSKVTREEHFQLHHQMHYSQQITVSLAQGYERTPSSPQPRFKSYAYTQAAYVSTSDPTRSPFPSQRLEAPEDKSFGSPLMETEVNLDSYQTALEEVLSWLLSAEDTLQAQGEISNDVEEVKEQFHTHEGYMMDLTSHQGRVGNVLQLGSQLIGSGKLSEDEETEVQEQMNLLNSRWECLRVASMEKQSNLHKVLMDLQNQQLKELNAWLTKTEERTRKMEKEPLGPDLEDLKRQIQQHKVLQEDLEQEQVRVNSLTHMVVVVDESSGDHATAALEEQLKVLGDRWANICRWTEDRWVLLQDVLLKWQRFTEEQCLFSTWLSDKEDALNKIPTSGFKDQSEMLSSLQKLAVLKTDLEKKKQTMDKLCSLNHDLLSTLKNTLVAQKMEAWLDNFAQRWDNLVQKLEKSSTQISQAVTTTQPSLTQTTVMETVTMVTTREQILVKHAQEELPPPPPQKKRQIIVDSEIKKRLDVDITELHSWITRSEAVLQSPEFAVYRKEGNFSDLKEKVNAIEREKAEKFRKLQDASRSAQALVEQMVNEGVNADSIKQASEQLNSRWIEFCQLLSERLNWLEYQNNIITFYNQLQQLEQMTTTAENWLKTQPTTPSEPTAVKSQLKNCKDEVNRLSALQPQIERLKIQSIALKEKGQGPMFLDADFVAFTNHFNQVFADMQAREKELQTILDTLPTVRYQETMSTILTWIQQSETKLSIPQVTVTEYEIMEQRLEELQALQSSLQEHQNDLNYLSTTVKEMSRKAPSHISQRYQSEFENIEGRWKKLSAQLNERCQKLEEQMAKLRKLQNHIKTLKKWMAEVDVFLKDEWPALGDSEILKKQLKQCRLLVSDIQTIQPSLNSVNEGGQKIKTEAEPEFASRLETELRELNTQWDYICRQVYARKEALKGGLDKTVSLQKDLSEMHEWMTQAEEEYLERDFEYKTPDELQTAVEEMKRAKEEAQQKEAKVQLLTESVNSVIAQAPPAAQEALRKELDTLTTNYQWLCTRLNGKCKTLEEVWACWHELLSYLEKANKWLNEVELKLKTTENIPGGAEEISEVLSSLENLMQHSEDNPNQIRILAQTLTDGGVMDELINEELETFNSRWRELHEEAVRRQKLLEQSIQSAQEIEKSLQLIQESLSSIDKQLAAYIADKVDAAQMPQEAQKIQSDLTSHEISLEEMKKHYQGKETAQRVLSQIEVAQKKMQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNALTEWLAATDLELTKRSAVEGMPSNLDSEVAWGKATQKETEKQKVHLKSITELGEALKTVLGKKETLVEDKLSLLNSNWIAVTSRAEEWLNLLLEYQKHMETFDQNVDHITKWIIQADALLDESEKKKPQQKEDMLKRLKAELNDIRPKVDSTRDQAANLMANRGDHCRKVIEPKISELNHRFAAISHRIKTGKASIPLKELEQFNSDIQKLLEPLEAEIQQGVNLKEEDFNKDMSEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVHRQAEGLSEDGAAMAVESTQIQLSKRWREIESKFAQFRRLNFAQIHTVHEESVMVMTEDMPLEISYVPSTYLTEITHVLQALSEVEQLLNAPDLCAKDFQDLFKQEESLKNIKDNLQQISGRIDVIHNKKAAALQSTTPPEKARLQEALSRLDFQWERVNKMYKDRQGQFDRSVEKWRRFHYDMKIFNQWLTEAEHFLKKTQIPENWEHAKYKWYLKELQDGIGQRQTVVRVLNATGEEIIQQSSKIDASILQEKLGSLNLRWQEVCKQLAERKKRLEEQKNILSEFQRDLNEFVLWLEEAGNISSIPLEPGNEQQLKEKLEEVKLLAEELPLRQGILKQLNETGGTVLVSAPISPEEQDKLENKLKQTNLQWIKVSRSLPEKQGEIEAHIKDLGQLEEQLNHLLLWLSPIRSQLEIYNQPNQTGPFDIKETEVAVQAKQPDVEGILSKGQNLYKEKPATEPVKRKLEDLSSEWKAVTHLLQELRAKWPGPVPGLTATGAPPSQTVALVTQPVVAKETAVSKLEMPSSLLLEVPALADFNRAWTELTDWLSLLDRVLKAQRVMVGDLEDINEMIIKQKATLQDLEQRRPQLEELITAAQNLKNKTSNQEARTIITDRIERIQSQWDEVQEHLQNRRQQLNEMLKDSTQWLEAKEEAEQVVGQARAKLETWKEGPYTMDAIQRKITETKQLAKDLRQWQINVDVANDLALKLLRDYSADDTRKVHMITENINASWASIHKRVSERETALEETHRLLQQFPLDLEKFLAWLTEAETTANVLQDATHKERLLEDSKGVRELMKQWQDLQGEIEAHTDIYHNLDENGQKILRSLEGSDDAVLLQRRLDNMNFKWSELRKKSLNIRSHLEASSDQWKRLHLSLQELLVWLQLKDDELSRQAPIGGDFPAVQKQNDIHRAFKRELKTKEPVIMSTLETVRIFLTEQPLEGLEKLYQEPRELPPEEKAQNVTRLLRKQAEEVNTEWEKLNLHSADWQRKIDEALERLQELQEATDELDLKLRQAEVIKGSWQPVGDLLIDSLQDHLEKVKALRGEIAPLKENVSHVSDLARQLTTLGIQLSPYNLSTLEDLNTRWKLLQVAVEDRIRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSSGSYLNDSISPNESIDDEHLLIQHYCQSLNQDSPLSQPRSPAQILISLESEERGELERILADLEEENRNLQAEYDRLKEQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSESMGEEDLLSPPQDSSTGLEEVMEQLNNSFPSSRGRNTPGKPMREDTM

SEQ ID NO: 72 - protein, 395 aa, Bos taurus
MSAADRMGARAVPGLRLALLLLMVLGTPKSGVQGEEGLDFPEYDGVDRVVNVNAKNYKNVFKKYEVLALLYHEPPEDDKASQRQFEMDELILELAAQVLEDKGVGFGMVDSEKDAAVAKKLGLTEEDSVYVFKGDEVIEYDGEFSADTLVEFLLDVLEDPVELIEGERELQAFENIEDDNKLIGYFKNKDSEHYKAYEDAAEEFHPYIPFFATFDSKVAKKLTLKLNEIDFYEAFMEEPVTIPDKPNSEEEIVSFVEAHKRSTLRKLKPESMYETWEDDLDGIHIVAFAEETDPDGYEFLETLKAVAQDNTDNPDLSIIWIDPDDFPLLVPYWEKTFNIDLSAPQIGVVNVTDADSVWMEMDDEEDLPSAEELEDWLEDVLEGEINTEDDDEEDD

SEQ ID NO: 73 - protein, 377 aa, Gallus gallus
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 74 - protein, 1937 aa, Gallus gallus
MMDMTEFGEAAPFLRKSEKELMMLQTVAFDGKKKCWVPDDKKAYVEAEITESSGGKVTVETTDGRTMTIKEDDVQSMNPPKFDMIEDMAMLTHLNEASVLYNLRKRYSNWMIYTYSGLFCVTINPYKWLPVYKSEVVAAYKGKRRSEAPPHIFSIADNAYHDMLRNRENQSMLITGESGAGKTVNTKRVIQYFATVAALGEPGKKSQPATKTGGTLEDQIIQANPALEAFGNAKTLRNDNSSRFGKFIRIHFGTTGKLSSADIEIYLLEKSRVIFQQPGERDYHIFYQILSGKKPELLDMLLVSTNPYDYHFCSQGVVTVDNLDDGEELMATDQAMDILGFVPDEKYGAYKLTGAIMHFGNMKFKQRPREEQAEADGTESADKAAYLMGINSSDLVKGLLHPRVKVGNEYVTKGQSVEQVLYAVGALSKAVYDRMFKWLVVRINKTLDTKLPRQFFIGVLDIAGFEIFDFNSFEQLCINYTNEKLQQFFNHHMFVLEQEEYKKEGIEWVFIDFGMDLQACIDLIEKPLGILSILEEECMFPKATDMTFKAKLYDNHLGKSPNLQKPRPDKKRKYEAHFELIHYAGSVPYNIIGWLEKNKDPLNETVVGIFQKSSNKLLASLFESYVGADSADQGGEKKRKKGASFQTVSSLHKENLNKLMTNLRSTAPHFVRCIIPNESKTPGEMDAFLVLHQLRCNGVLEGIRICRKGFPNRVLYADFKQRYRILNPGAIPEDKFVDSRKAAEKLLASLDIDHNQYRFGHTKVFFKAGLLGHLEEMRDERLAKILTMIQARARGRLMRIEFQKIVERRDALLVIQWNIRAFMAVKNWPWMKLFFKIKPLLKSAETEKEMANMKEEFLKLKEALEKSEARRKELEEKQVSLVQEKNDLLLQLQAEQDTLADAEERCDLLIKSKIQLEAKVKELTERVEDEEEMNSELTSKKRKLEDECSELKKDIDDLEITLAKVEKEKHATENKVKNLTEEMATLDENISKLTKEKKSLQEAHQQVLDDLQAEEDKVNTLSKAKVKLEQQVDDLEGSLEQEKKVRMDLERAKRKLEGDLKLTQESVMDLENDKLQMEEKLKKKEFEMSQLNSKIEDEQAIVMQLQKKIKELQARIEELEEELEAERAARAKVEKQRSDLARELEVLSERLEEAGGATAAQLEMNKKREAEFLKLARDLEEATLHYEATAAALRKKHADSVAEMGEQLDNLQRVKQKLEKEKSELKMEVDDLTSNMEQTVKGKANAEKLCRTYEDHLNETKTKLDEMTRLMNDLTTQKTKLQSENGEFVRQLEEKESLISQLSRGKTSFTQQIEELRRQLEEETKSKNALAHALQAARHDCDLLREQYEEEQEAKAELQRALSKGNAEVAQWRTKYETDAIQRTEELEDAKKKLAARLQEAEEAIEAANAKCSSLEKTKHRLQNELEDMMIDLEKANSAAASLDKKQRGFDKIINDWKQKYEESQAELEASQKEARSLSTELFKLKNAYEETLDHLETLKRENKNLQEEISDLTNQISEGNKNLHEIEKVKKQVEQEKSEVQLALEEAEGALEHEESKTLRFQLELSQLKADFERKLAEKDEEMENIRRNQQRTIDSLQSTLDSEARSRNEAIRLKKKMEGDLNEMEIQLSHANRHAAEATKSARGLQTQIKELQVQLDDLGHLNEDLKEQLAVSDRRNNLLQSELDELRALLDQTERARKLAEHELLEATERVNLLHTQNTSLINQKKKLEGDISQMQNEVEESIQECRNAEEKAKKAITDAAMMAEELKKEQDTSAHLERMKKNMEQTIKDLQKRLDEAEQIALKGGKKQIQKLESRVRELENELENELRRNSDAQKGARKFERRIKEVTYQSEEDKKNLARMQDLIDKLQLKVKSYKHQAEEAEAQANLYLSKYRKQQHDLDDAEERAEIAESQVNKLRSKSRDIGMKKVHEEE

SEQ ID NO: 75 - protein, 1272 aa, Gallus gallus
MPEPAKKAVSAFTKKPKTTEVAAGSTAVFEAETEKTGIKVKWQRAGTEITDSEKYAIKAEGNKHSLTISNVGKDDEVTYAVIAGTSKVKFELKVKEPEKSEPVAPAEASPAPAASELPAPPVESNQNPEVPPAETQPEEPVDPIGLFVTRPQDGEVTVGGNITFTAKVAGESLLKKPSVKWFKGKWMDLASKVGKHLQLHDNYDRNNKVYTFEMEIIEANMTFAGGYRCEVSTKDKFDSSNFNLIVNEAPVSGEMDIRAAFRRTSLAGGGRRMTSAFLSTEGLEESGELNFSALLKKRDSFLRTANRGDGKSDSQPDVDVWEILRKAPPSEYEKIAFQYGITDLRGMLKRLKRIKKEEKKSTAFLKKLDPAYQVDKGQKIKLMVEVANPDADVKWLKNGQEIQVSGSKYIFEAIGNKRILTINHCSLADDAAYECVVAEEKSFTELFVKEPPILITHPLEDQMVMVGERVEFECEVSEEGATVKWEKDGVELTREETFKYRFKKDGKKQYLIINESTKEDSGHYTVKTNGGVSVAELIVQEKKLEVYQSIADLTVKARDQAVFKCEVSDENVKGIWLKNGKEVVPDERIKISHIGRIHKLTIEDVTPGDEADYSFIPQGFAYNLSAKLQFLEVKIDFVPREEPPKIHLDCLGQSPDTIVVVAGNKLRLDVPISGDPTPTVIWQKVNKKGELVHQSNEDSLTPSENSSDLSTDSKLLFESEGRVRVEKHEDHCVFIIEGAEKEDEGVYRVIVKNPVGEDKADITVKVIDVPDPPEAPKISNIGEDYCTVQWQPPTYDGGQPVLGYILERKKKKSYRWMRLNFDLLKELTYEAKRMIEGVVYEMRIYAVNSIGMSRPSPASQPFMPIAPPSEPTHFTVEDVSDTTVALKWRPPERIGAGGLDGYIVEYCKDGSAEWTPALPGLTERTSALIKDLVTGDKLYFRVKAINLAGESGAAIIKEPVTVQEIMQRPKICVPRHLRQTLVKKVGETINIMIPFQGKPRPKISWMKDGQTLDSKDVGIRNSSTDTILFIRKAELHHSGAYEVTLQIENMTDTVAITIQIIDKPGPPQNIKLADVWGFNVALEWTPPQDDGNAQILGYTVQKADKKTMEWYTVYDHYRRTNCVVSDLIMGNEYFFRVFSENLCGLSETAATTKNPAYIQKTGTTYKPPSYKEHDFSEPPKFTHPLVNRSVIAGYNTTLSCAVRGIPKPKIFWYKNKVDLSGDAKYRMFSKQGVLTLEIRKPTPLDGGFYTCKAVNERGEAEIECRLDVRVPQ

SEQ ID NO: 76 - protein, 1271 aa, Gallus gallus
MPEPAKKAVSAFTKKPKTTEVAAGSTAVFEAETEKTGIKVKWQRAGTEITDSEKYAIKAEGNKHSLTISNVGKDDEVTYAVIAGTSKVKFELKVKEPEKSEPVAPAEASAPAASELPAPPVESNQNPEVPPAETQPEEPVDPIGLFVTRPQDGEVTVGGNITFTAKVAGESLLKKPSVKWFKGKWMDLASKVGKHLQLHDNYDRNNKVYTFEMEIIEANMTFAGGYRCEVSTKDKFDSSNFNLIVNEAPVSGEMDIRAAFRRTSLAGGGRRMTSAFLSTEGLEESGELNFSALLKKSSSFLRTANRGDGKSDSQPDVDVWEILRKAPPSEYEKIAFQYGITDLRGMLKRLKRIKKEEKKSTAFLKKLDPAYQVDKGQKIKLMVEVANPDADVKWLKNGQEIQVSGSRYIFEAIGNKRILTINHCSLADDAAYECVVAEEKSFTELFVKEPPILITHPLEDQMVMVGERVEFECEVSEEGATVKWEKDGVELTREETFKYRFKKDGKKQYLIINESTKEDSGHYTVKTNGGVSVAELIVQEKKLEVYQSIADLTVKARDQAVFKCEVSDENVKGIWLKNGKEVVPDERIKISHIGRIHKLTIEDVTPGDEADYSFIPQGFAYNLSAKLQFLEVKIDFVPREEPPKIHLDCLGQSPDTIVVVAGNKLRLDVPISGDPTPTVIWQKVNKKGELVHQSNEDSLTPSENSSDLSTDSKLLFESEGRVRVEKHEDHCVFIIEGAEKEDEGVYRVIVKNPVGEDKADITVKVIDVPDPPEAPKISNIGEDYCTVQWQPPTYDGGQPVLGYILERKKKKSYRWMRLNFDLLKELTYEAKRMIEGVVYEMRIYAVNSIGMSRPSPASQPFMPIAPPSEPTHFTVEDVSDTTVALKWRPPERIGAGGLDGYIVEYCKDGSAEWTPALPGLTERTSALIKDLVTGDKLYFRVKAINLAGESGAAIIKEPVTVQEIMQRPKIWLPRHLRQTLVKKVGETINIMIPFQGKPRPKISWMKDGQTLDSKDVGIRNSSTDTILFIRKAELHHSGAYEVTLQIENMTDTVAITIQIIDKPGPPQNIKLADVWGFNVALEWTPPQDDGNAQILGYTVQKADKKTMEWYTVYDHYRRTNCVVSDLIMGNEYFFRVFSENLCGLSETAATTKNPAYIQKTGTTYKPPSYKEHDFSEPPKFTHPLVNRSVIAGYNTTLSCAVRGIPKPKIFWYKNKVDLSGDAKYRMFSKQGVLTLEIRKPTPFDGGFYTCKAVNERGEAEIECRLDVRVPQ

SEQ ID NO: 77 - protein, 208 aa, Gallus gallus
MAEEEEPKPPPLRRKSSANYRGYAVEPHAKRQSKISASRKLQLKTLLLQRAKRELEREEQERAGEKQRHLGELCPPPELEGLGVAQLQELCRELHARIGRVDEERYDMGTRVSKNMAEMEELRRRVAGGRFVRPALRRVRLSADAMMAALLGSKHRVGTDLRAGLRQVRKDDAEKESREVGDWRKNVDALSGMEGRKKKFEAPGGGQG

SEQ ID NO: 78 - protein, 219 aa, Gallus gallus
MKQKHQEKGESKPKPKPFMPNLVPPKIPDGERLDFDDIHRKRMEKDLNELQALIEAHFESRKKEEEELISLKDRIEQRRAERAEQQRIRSEREKERQARMAEERARKEEEEARKKAEEEARKKKAFSNMLHFGGYMQKSEKKGGKKQTEREKKKKILSERRKPLNIDHLSEDKLRDKAKELWQTIRDLEAEKFDLQEKFKRQKYEINVLRNRVSDHQKV

SEQ ID NO: 79 - protein, 197 aa, Sus scrofa
MPPKKPEPKKEAAKAAPAPAPAPAPAPAPPPEPAKEPTFDPKSIKIDFTADQIEEFKEAFSLFDRTPTGEMKITYGQCGDVLRALGQNPTNAEVLRVLGKPKPEEMNAKMLDFETFLPILQHISRNKEQGTYEDFVEGLRVFDKESNGTVMGAELRHVLATLGEKMTEAEVEQLLAGQEDANGCINYEAFVKHIMSG

SEQ ID NO: 80 - protein, 1931 aa, Gallus gallus
MEALLGAAAPFLRAPEGPRPTPAGDTRGLCFVPHPQLEFIRARVTARAGNGVTVTTEMGETLTVPEADVHPQNPPKFDRIEDMAMLTFLHEPAVLYNLKERYASWMIYTYSGLFCVTVNPYKWLPVYNAEVVAAYRGKKRTEVPPHIFSISDNAYQNMLTDRENQSILITGESGAGKTVNTKRVIQYFASIAAIGHRKKEVANSSKGTLEDQIIQANPALEAFGNAKTVRNDNSSRFGKFIRIHFGATGKLASADIETYLLEKSRVIFQLKAERNYHIFYQILSNKKPELLEMLLITNNPYDYSYVSQGEVTVASIDDSEELLATDSAFDVLGFTAEEKAGVYKLTGAIMHFGNMKFKQKQREEQAEPDGTEDCDKSAYLMGLNSADLLKGLCHPRVKVGNEYVTKGQSVQQVYYSIGALAKAVYEKMFNWMVVRINNSLETKQPRQYFIGVLDIAGFEIFDFNSFEQLCINFTNEKLQQFFNHHMFVLEQEEYKKEGIEWEFIDFGMDLQACIDLIEKPMGIMSILEEECMFPKASDMTFKAKLFDNHLGKSANFGKPRNVKGKSEAHFSLIHYAGTVDYNIIGWLEKNKDPLNETVVGLYQKSALKLLASLFSNYAGADAGGDGGKGKGAKKKGSSFQTVSALHRENLNKLMANLKTTHPHFVRCLIPNERKEPGVMDNPLVMHQLRCNGVLEGIRICRKGFPNRILYGDFRQRYRIPNPTAIPEGQFIDSRKGAEKLLGSLDIDHNQYKFGHTKVFFKAGLLGLLEEMRDERLSLIITRIQAQARGQLMRIEFKKILERRDALLVIQWNIRAFMGVKNWPWMKLYFKIKPLLKSAETEKEMQTMKEEFGHLKEALEKSAARRKELEEKMVSMLQEKNDLQLQVQAEQDNLADAEERCDQLIKNKIQLEAKVKEMTERLEEEEEMNAELAAKKRKLEDECSELKKDIDDLELSLAKVEKEKHATENKVKNLTEEMAGLDENITKLTKEKKILQESHQQALDDLQAEEDKVNTLAKAKGKLEQQVDDLESSLEQEKKIRMDLERAKRKLEGDLKLAQESIMDLENDKQQLEERLKKKDFELNTLNARIEDEQAISAQLQKKLKELQARIEELEEELEAERTGRAKVEKLRSELLQELEETSERLEEAGGATSVQLELNKKREAEFQKLRRDLEEATLQHEATAATLRKKHADSVAELSEQLDNLQRVKQKLEKEKSELKLELDDVNSNTEQLIKAKTNLEKMCRTTEDQMNEHRSKLEEAQRTVTDLSTQRAKLQTENSELSRQLEEKEAFINQLTRGKLTYTQQLEDLKRQLEEEAKAKNALAHALQSAQHDCDLLREQYEEEMEAKAELQRALSKANSEVAQWRTKYETDAIQRTEELEEAKKKLAQRLQEAEEAVEAVNAKCSSLEKTKHRLQNEIEDLMADVERSNAAAAALDKKQRNFDKILSEWKQKFEESQTELEASQKEARSLSTELFKLKNAYEESLEHLETFKRENKNLQEEILDLTEQLGASQKSIHELEKVRKQLDAEKLELQAALEEAEASLEHEEGKILRAQLEFNQVKADYERKLAEKDEEIEQSKRNHLRVVDSLQTSLDAETRSRNEALRLKKKMEGDLNEMEIQLSHANRTAAEAQKQVKALQGYLKDTQLQLDDVVRANEDLKENIAIVERRNNLLQSELEELRAMVEQSERARKLAEQELIEASERVQLLHSQNTSLINQKKKMEADISQLQTEVEEAIQECRNAEEKAKKAITDAAMMAEELKKEQDTSAHLERMKKNMEQTVKDLQLRLDEAEQLALKGGKKQLQKLEVRVRELENELEAEQKRNAESIKGLRKSERRVKELSYQTEEDRKNMVRLQDLVDKLQLKVKAYKRQAEEAEEQANSNLAKFRKVQHELDEAEERADMAESQVNKLRARSRDIGAKKGLNEE

SEQ ID NO: 81 - protein, 1941 aa, Gallus gallus
MSMLDMSEFGEAAEYLRKSYTEQLKLQTIPFDGKKRAWIPDEKEAYIEVEIKESTGGKVTVETKDKQTRVVKEDELQAMNPPKFDMIEDMAMLTHLNEASVLYNLKRRYSHWMIYTYSGLFCVTINPYKWLPVYTAPVVAAYKGKRRSEAPPHIYSIADNAYNDMLRNRENQSMLITGESGAGKTVNTKRVIQYFAIVAALGDTPGKKVAALATKTGGTLEDQIIEANPAMEAFGNAKTIRNDNSSRFGKFIRIHFGPSGKLASADIDIYLLEKSRVIFQQPKERSYHIYYQILSGKKPELQDMLLLSLNPYDYHFCSQGVTTVDNLDDGEELMATDHAMDILGFSNDEKYGSYKIVGAIMHFGNMKFKQKQREEQAEADGTESADKAAYLMGISSADLIKGLLHPRVKVGNEYVTKGQNVEQVVYAVGALAKATYDRMFKWLVTRINKTLDTKLARQFFIGVLDIAGFEIFDFNSFEQLCINFTNEKLQQFFNHHMFVLEQEEYKKEGIEWVFIDFGLDLQACIDLIEKPLGILSILEEECMFPKASDMSFKAKLYDNHLGKSPNFQKPRPDKKRKYEAHFELVHYAGVVPYNIIGWLDKNKDPLNETVVAVFQKSQNKLLASLYENYVGSSSEEPHKPGSKEKRKKAASFQTVSQLHKENLNKLMTNLRSTQPHFVRCIIPNETKTPGAMDAFLVLHQLRCNGVLEGIRICRKGFPNRILYADFKQRYRILNPAAIPDDKFVDSRKATEKLLSSLELDHSQYKFGHTKVFFKAGLLGMLEEMRDERLAKILTMLQARIRGHLMRIEYQKIISRREALYTIQWNIRAFNAVKNWSWMKLFFKIKPLLKSAQTEKEMSTLKEEFQKLKEALEKSEAKRKELEEKQVSMIQEKNDLALQLQAEQDNLADAEERCDLLIKSKIQLEAKVKELTERVEDEEEMNADLTAKKRKLEDECAELKKDIDDLEITLAKVEKEKHATENKVKNLIEEMAALDEIIAKLTKEKKALQEAHQQALDDLQAEEDKVNTLTKAKVKLEQQVDDLESSLEQEKKIRMDLERAKRKLEGDLKLTQESVMDLENDKQQLEEKLKKKDFEMSQLNSRIEDQQVTEAQLQKKIKELQARIEELEEELEAERAARAKVEKQRAEVSRELEELSERLEEAGGATSAQLEMNKKREVEFLKLRRDLEEATLQHESTAAALRKKHADSVAELSEQIDNLQRVKQKLEKEKSEMKMEVDDLSSNIEYLTKNKANAEKLCRTYEDQLSEAKSKVDELQRQLTDVSTQRGRLQTENGELSRLLEEKESFINQLSRGKTSFTQTIEELKRQLEEETKSKNALAHALQASRHDCDLLREQYEEEVEAKSELQRNLSKANAEVAQWRTKYETDAIQRTEELEEAKKKLAIRLQEAEEAVEAAHAKCSSLEKTKHRLQTEIEDLSVDLERANSACAALDKKQRNFDRILAEWKQKYEETQAELEASQKESRSLSTELFKLKNAYEESLDNLETLKRENKNLQEEIADLTDQISMSGKTIHELEKLKKALENEKSDIQAALEEAEGALEHEESKTLRIQLELNQIKADVDRKLAEKDEEFENLRRNHQRAMDSMQATLDAEARAKNEAVRLRKKMEGDLNEMEIQLSHANRQAAEFQKLGRQLQAQIKDLQIELDDTQRQNDDLKEQAAALERRNNLLLAEVEELRAALEQAERSRKLAEQELLEATERVNLLHSQNTGLINQKKKLETDISQLSSEVEDAVQECRNAEEKAKKAITDAAMMAEELKKEQDTSAHLERMKKNMEQTIKDLQMRLDEAEQIALKGGKKQIQKLEARVRELEGELDMEQKKMAEAQKGIRKYERRIKELSYQTEEDRKNLTRMQDLIDKLQSKVKSYKRQFEEAEQQANSNLVKYRKVQHELDDAEERADIAETQVNKLRARTKEVITFKHE

SEQ ID NO: 82 - protein, 1935 aa, Sus scrofa
MVDAEMAAFGEAAPYLRKSEKERLEAQTRPFDLKKDVYVPDDKEEFVKAKILSREGGKVTAETEHGKTVTVKEDQVLQQNPPKFDKIEDMAMLTFLHEPAVLYNLKERYASWMIYTYSGLFCVTINPYKWLPVYNAEVVAAYRGKKRSEAPPHIFSISDNAYQYMLTDRENQSILITGESGAGKTVNTKRVIQYFAVIAAIGDRSKKEQTPGKGTLEDQIIQANPALEAFGNAKTVRNDNSSRFGKFIRIHFGATGKLASADIETYLLEKSRVIFQLKAERDYHIFYQILSNKKPELLDMLLITNNPYDYAFISQGETTVASIDDAEELMATDNAFDVLGFTSEEKNSMYKLTGAIMHFGNMKFKLKQREEQAEPDGTEEADKSAYLMGLNSADLLKGLCHPRVKVGNEYVTKGQNVQQVMYATGALAKAVYEKMFNWMVTRINTTLETKQPRQYFIGVLDIAGFEIFDFNSFEQLCINFTNEKLQQFFNHHMFVLEQEEYKKEGIEWEFIDFGMDLQACIDLIEKPMGIMSILEEECMFPKATDMTFKAKLYDNHLGKSNNFQKPRNIKGRPEAHFALIHYAGTVDYNIIGWLQKNKDPLNETVVDLYKKSSLKLLSNLFANYAGADTPVEKGKGKAKKGSSFQTVSALHRENLNKLMTNLRSTHPHFVRCIIPNETKSPGVIDNPLVMHQLRCNGVLEGIRICRKGFPNRILYGDFRQRYRILNPAAIPEGQFIDSRKGAEKLLGSLDIDHNQYKFGHTKVFFKAGLLGLLEEMRDERLSRIITRIQAQSRGVLSRMEFKKLLERRDSLLIIQWNIRAFMSVKNWPWMKLYFKIKPLLKSAETEKEMATMKEEFGRLKEALEKSEARRKELEEKMVSLLQEKNDLQLQVQAEQDNLADAEERCDQLIKNKIQLEAKVKEMTERLEDEEEMNAELTAKKRKLEDECSELKRDIDDLELTLAKVEKEKHATENKVKNLTEEMAGLDEIIAKLTKEKKALQEAHQQALDDLQAEEDKVNTLTKAKVKLEQHVDDLEGSLEQEKKVRMDLERAKRKLEGDLKLTQESIMDLENDKQQLDERLKKKDFELNALNARIEDEQALGSQLQKKLKELQARIEELEEELEAERTARAKVEKLRSDLSRELEEISERLEEAGGATSVQIEMNKKREAEFQKMRRDLEEATLQHEATAAALRKKHADSVAELGEQIDNLQRVKQKLEKEKSEFKLELDDVTSNMEQIIKAKANLEKMCRTLEDQMNEHRSKAEETQRSVNDLTSQRAKLQTENGELSRQLDEKEALISQLTRGKLTYTQQLEDLKRQLEEEVKAKNALAHALQSARHDCDLLREQYEEETEAKAELQRVLSKANSEVAQWRTKYETDAIQRTEELEEAKKKLAQRLQDAEEAVEAVNAKCSSLEKTKHRLQNEIEDLMVDVERSNAAAAALDKKQRNFDKILAEWKQKYEESQSELESSQKEARSLSTELFKLKNAYEESLEHLETFKRENKNLQEEISDLTEQLGSSGKTIHELEKVRKQLEAEKLELQSALEEAEASLEHEEGKILRAQLEFNQIKAEMERKLAEKDEEMEQAKRNHLRVVDSLQTSLDAETRSRNEALRVKKKMEGDLNEMEIQLSHANRMAAEAQKQVKSLQSLLKDTQIQLDDAVRANDDLKENIAIVERRNNLLQAELEELRAVVEQTERSRKLAEQELIETSERVQLLHSQNTSLINQKKKMEADLSQLQTEVEEAVQECRNAEEKAKKAITDAAMMAEELKKEQDTSAHLERMKKNMEQTIKDLQHRLDEAEQIALKGGKKQLQKLEARVRELENELEAEQKRNAESVKGMRKSERRIKELTYQTEEDRKNLLRLQDLVDKLQLKVKAYKRQAEEAEEQANTNLSKFRKVQHELDEAEERADIAESQVNKLRAKSRDIGTKGLNEE

SEQ ID NO: 83 - protein, 354 aa, Bos taurus
MEAATAPEAALRPTLKVKEASPADADGPQASPRRGTGSPLSQLLPPIEEHPKIWLPRALRQTYIRKVGDTVNLLIPFQGKPKPQAIWTRDGCALDTSRVSVRNGERDSILFIREAQRADSGRYQLSVQLGGLEATATIDILVIERPGPPQSIKLVDVWGSSATLEWTPPQDTGNAALLGYTVQKADTKSGLWFTVLERCRRASCTVPNLIVGNSYTFRVFAENQCGLSENAPITADLAHIQKAATVYRTEGFAQRDFSEAPKFTQPLADCTTVIGYDTQLFCCVRASPKPKIIWLKNKMDIQGNPKYRALTHLGICSLEIRKPGPFDGGIYTCKAVNPLGEASVDCRVDVKAPN

SEQ ID NO: 84 - protein, 471 aa, Sus scrofa
MSQAYSSSQRVSSYRRTFGGAPSFPLGSPLSSPVFPRAGFGTKGSSSSVTSRVYQVSRTSGGAGGLGPLRASRLGATRVPSSSYGAGELLDFSLADAVNQEFLTTRTNEKVELQELNDRFANYIEKVRFLEQQNAALAAEVNRLKGREPTRVAEIYEEELRELRRQVEVLTNQRARVDVERDNLLDDLQRLKAKLQEEIQLKEEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEASGYQDNIARLEEEIRHLKDEMARHLREYQDLLNVKMALDVEIATYRKLLEGEESRINLPIQTFSALNFRETSPEQRGSEVHTKKTVMIKTIETRDGEVVSEATQQQHEVL

SEQ ID NO: 85 - protein, 566 aa, Sus scrofa
MSEHSRNSDQEEPFDREIDEDEILANLSPEELKELQSEMDVMAPDPRLPVGMIQKDQTDKPPTGNFDHKSLVDYMYWQKASRRMLEDERVPVTFVPSEEKPQEQRKEIDKGNKNMSQYLKEKLNNEIAAHKRESKSSDNEQETNDDDEDNEDDEDDEEDDAEDEEDEGDESDEKTKGEEEGEVKEPIRNGESNCQQVPNKAFEEQKDRPEAQEKFEKKISKLDPKKLALDTSFLKVSARPSGNQTDLDGSLRRVRQNDPDMKELNLNNIENIPKEMLLDFVNAMKKNKHIKTFSLANVGADESVAFALANMLRENRSITTLNIESNFITGKGIVAIMRCLQFNETLTELRFHNQRHMLGHHAEMEISRLLKANTTLLKMGYHFELPGPRMVVTNLLTRNQDKQRQKRQEEQKQQQLKEQKKLIAMLENGLGLPPGMWEMLGGPMPDSQMQEFLQPPPPKSLHPQTAPFSRRNEVMAKPAQPPKYRTDPDSFRVVKLKRIQRKSRMPEAREPPEKTNLKDVIKTLKPVPRNRPPPLVEITPRDQLLNDIRHSNIAYLKPVQLPKELA

SEQ ID NO: 86 - protein, 284 aa, Bos taurus
MDAIKKKMQMLKLDKENALDRAEQAEADKKAAEDRSKQLEDELVSLQKKLKATEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSI

SEQ ID NO: 87 - protein, 284 aa, Gallus gallus
MEAIKKKMQMLKLDKENAIDRAEQAETDKKAAEDKCKQVEDELVALQKKLKGTEDELDKYSEALKDAQEKLEQAEKKATDAEGEVAALNRRIQLVEEELDRAQERLATALQKLEEAEKAADESERGMKVIENRAMKDEEKMEIQEMQLKEAKHIAEEADRKYEEVARKLVILEGELERAEERAEVSEVKCSDLEEELKNVTNNLKSLEAQSEKYSEKEDKYEEEIKILSDKLKEAETRAEFAERTVAKLEKSIDDLEDELYAQKLKYKAISEELDHALNDMTSL

SEQ ID NO: 88 - protein, 332 aa, Gallus gallus
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSMARRALQESSFGEMGHLPWFPEPQRVYPVSGRVDFFSGMGGLTYGLKRT

SEQ ID NO: 89 - protein, 552 aa, Gallus gallus
MSTFGYRRELSKYEDIDEDELLASLTEEELKELERELEDIEPDRNLPVGQRQKSLTEKTPTGTFSREALMAYWERETRKLLEKERLGACEKDSEQEEDNSEDIQEECFTESNSEVSEEAYTEEDDEEEEEEEEEEEDEDDSDDEDEEKQNSAASERPVNCEDGRSSSHVRHKKCSNAKNSENLFNGHDGKDTENLSFKSSAIHPCGNPTVIEDALEKVRSNDPETTEVNLNNIENITSQMLIQFSQALRDNTVVKSFSLANTHADDNVAIAIAGMLKVNQHITSLNIESNFITGKGVLAIMRALQHNKVLTELRFHNQRHIMGSQVEMDIVKLLKENTTLVKLGYHFDLAGPRMSMTSILTRNMDKQRQKRMQEQRQQEYGCDGAINPKTKVLQKGTPRSSPYTSPKSSPWSSPKLPRKSAPAKSQPPAPAPPPPPPPPPPPPPPPPPVIPDKKAPTRNIAEVIKQQESSRKALQNGQKKKKGKKGKKHENSILKEIKDSLKSVSDRKSEEGSRPSTRPSTPQRSLHDNLMEAIRASSIKQLRRVEVPEALR

SEQ ID NO: 90 - protein, 365 aa, Sus scrofa
MPQNVILPGPAPWGFRLSGGIDFNQPLIITRITPGSKAAAANLCPGDVILAIDGYGTESMTHADAQDRIKAAAHQLCLKIDRAETRLWSPQVTEDGKAHPFKINLESEPQDVNYFEHKHNIRPKPFIIPGRSSGCSTPSGIDGGSGRSTPSSVSTLSTICPGDLKVAAKMAPNIPLEMELPGVKIVHAQFNTPMQLYSDDNIMETLQGQVSTALGETPSMSEPTTASVPPQSDVYRMLHDNRNEPTQPRQSGSFRVLQELVNDGPDDRPAGTRSVRAPVTKIHGGAGGTQKMPLCDKCGSGIVGAVVKARDKYRHPECFVCADCNLNLKQKGYFFVEGELYCETHARARMRPPEGYDTVTLYPKA

SEQ ID NO: 91 - protein, 431 aa, Bos taurus
MTPSKSSKAPFTFLPSAFLGKKHFAQMKRAHLFVVGVYLLSSCRAEEGLNFPTYDGKDRVVSLTEKNFKQVLKKYDLLCLYYHEPLSSDKVVQKQFQLKEIVLELVAQVLEHKDIGFVMVDAKKEAKLAKKLGFDEEGSLYILKGDRTIEFDGEFAADVLVEFLLDLIEDPVEIINSKLEVQAFERIEDHIKLIGFFKSEESEHYKAFEEAAEHFQPYIKFFATFDKGVAKKLSLKMNEVDFYEPFMDEPIAIPDKPYTEEELVEFVKEHQRPTLRRLRPEDMFETWEDDLNGIHIVAFAERSDPDGYEFLEILKQVARDNTDNPDLSIVWIDPDDFPLLVAYWEKTFKIDLFKPQIGVVNVTDADSVWMDIPDDDDLPTAEELEDWIEDVLSGKINTEDDDNDDEEDEDDDDDDDNSDEEDNDDSDDDDE

SEQ ID NO: 92 - protein, 377 aa, Gallus gallus
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILSERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 93 - protein, 376 aa, Gallus gallus
MCEEETTALVCDNGSGLCKAGFAGDDAPRAVFPSIVGRPRHQGVMVGMGQKDSYVGDEAQSKRGILTLKYPIEHGIITNWDDMEKIWHHSFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKPEYDEAGPSIVHRKCF

SEQ ID NO: 94 - protein, 172 aa, Gallus gallus
MSSKRAKTKTTKKRPQRATSNVFAMFDQSQIQEFKEAFNMIDQNRDGFIDKEDLHDMLASLGKNPTDEYLDAMMNEAPGPINFTMFLTMFGEKLNGTDPEDVIRNAFACFDEEATGFIQEDYLRELLTTMGDRFTDEEVDELYREAPIDKKGNFNYIEFTRILKHGAKDKDD

SEQ ID NO: 95 - protein, 1979 aa, Gallus gallus
MSQKPLSDDEKFLFVDKNFVNNPLAQADWSAKKLVWVPSEKHGFEAASIKEEKGDEVTVELQENGKKVTLSKDDIQKMNPPKFSKVEDMAELTCLNEASVLHNLRERYFSGLIYTYSGLFCVVINPYKQLPIYSEKIIDMYKGKKRHEMPPHIYAIADTAYRSMLQDREDQSILCTGESGAGKTENTKKVIQYLAVVASSHKGKKDTSITQGPSFSYGELEKQLLQANPILEAFGNAKTVKNDNSSRFGKFIRINFDVTGYIVGANIETYLLEKSRAIRQAKDERTFHIFYYLIAGASEQMRNDLLLEGFNNYTFLSNGHVPIPAQQDDEMFQETLEAMTIMGFTEEEQTSILRVVSSVLQLGNIVFKKERNTDQASMPDNTAAQKVCHLMGINVTDFTRSILTPRIKVGRDVVQKAQTKEQADFAIEALAKAKFERLFRWILTRVNKALDKTKRQGASFLGILDIAGFEIFEINSFEQLCINYTNEKLQQLFNHTMFILEQEEYQREGIEWNFIDFGLDLQPCIELIERPTNPPGVLALLDEECWFPKATDTSFVEKLIQEQGNHAKFQKSKQLKDKTEFCILHYAGKVTYNASAWLTKNMDPLNDNVTSLLNQSSDKFVADLWKDVDRIVGLDQMAKMTESSLPSASKTKKGMFRTVGQLYKEQLTKLMTTLRNTNPNFVRCIIPNHEKRAGKLDAHLVLEQLRCNGVLEGIRICRQGFPNRIVFQEFRQRYEILAANAIPKGFMDGKQACILMIKALELDPNLYRIGQSKIFFRTGVLAHLEEERDLKITDVIIAFQAQCRGYLARKAFAKRQQQLTAMKVIQRNCAAYLKLRNWQWWRLFTKVKPLLQVTRQEEEMQAKDEELQRTKERQQKAEAELKELEQKHTQLCEEKNLLQEKLQAETELYAEAEEMRVRLAAKKQELEEILHEMEARIEEEEERSQQLQAEKKKMQQQMLDLEEQLEEEEAARQKLQLEKVTADGKIKKMEDDILIMEDQNNKLTKERKLLEERVSDLTTNLAEEEEKAKNLTKLKNKHESMISELEVRLKKEEKSRQELEKIKRKLEGESSDLHEQIAELQAQIAELKAQLAKKEEELQAALARLEDETSQKNNALKKIRELESHISDLQEDLESEKAARNKAEKQKRDLSEELEALKTELEDTLDTTATQQELRAKREQEVTVLKRALEEETRTHEAQVQEMRQKHTQAVEELTEQLEQFKRAKANLDKTKQTLEKDNADLANEIRSLSQAKQDVEHKKKKLEVQLQDLQSKYSDGERVRTELNEKVHKLQIEVENVTSLLNEAESKNIKLTKDVATLGSQLQDTQELLQEETRQKLNVTTKLRQLEDDKNSLQEQLDEEVEAKQNLERHISTLTIQLSDSKKKLQEFTATVETMEEGKKKLQREIESLTQQFEEKAASYDKLEKTKNRLQQELDDLVVDLDNQRQLVSNLEKKQKKFDQMLAEEKNISSKYADERDRAEAEAREKETKALSLARALEEALEAKEELERTNKMLKAEMEDLVSSKDDVGKNVHELEKSKRTLEQQVEEMKTQLEELEDELQAAEDAKLRLEVNMQAMKSQFERDLQARDEQNEEKRRQLLKQLHEHETELEDERKQRALAAAAKKKLEVDVKDLESQVDSANKAREEAIKQLRKLQAQMKDYQRDLDDARAAREEIFATARENEKKAKNLEAELIQLQEDLAAAERARKQADLEKEEMAEELASANSGRTSLQDEKRRLEARIAQLEEELDEEHSNIETMSDRMRKAVQQAEQLNNELATERATAQKNENARQQLERQNKELRSKLQEMEGAVKSKFKSTIAALEAKIASLEEQLEQEAREKQAAAKTLRQKDKKLKDALLQVEDERKQAEQYKDQAEKGNLRLKQLKRQLEEAEEESQRINANRRKLQRELDEATESNDALGREVAALKSKLRRGNEPVSFAPPRRSGGRRVIENATDGGEEEIDGRDGDFNGKASE

SEQ ID NO: 96 - protein, 151 aa, Gallus gallus
MCDFSEEQTAEFKEAFQLFDRTGDGKILYSQCGDVMRALGQNPTNAEVMKVLGNPKSDEMNLKTLKFEQFLPMMQTIAKNKDQGCFEDYVEGLRVFDKEGNGTVMGAEIRHVLVTLGEKMTEEEVEQLVAGHEDSNGCINYEELVRMVLSG

SEQ ID NO: 97 - protein, 462 aa, Gallus gallus
QSYSSSQRVSSYRRTFGGGTSPVFPRASFGSRGSGSSVTSRVYQVSRTSAVPTLSTFRTTRVTPLRTYGSAYQGAGELLDFSLADAMNQEFLQTRTNEKVELQELNDRFANYIEKVRFLEQQNALMVAEVNRLRGKQPTRVAEMYEEELRELRRQVDALTGQRARVEVERDNLLDNLQKLKQKLQEEIQLKQEAENNLAAFRADVDAATLARIDLERRIESLQEEIAFLKKVHEEEIRELQAQLQEQHIQVEMDISKPDLTAALRDIRAQYESIAAKNIAEAEEWYKSKVSDLTQAANKNNDALRQAKQEMLEYRHQIQSYTCEIDALKGTNDSLMRQMREMEERFAGEAGGYQDTIARLEEEIRHLKDEMARHLREYQDLLNVKMALDVEIATYRKLLEGEENRISIPMHQTFASALNFRETSPDQRGSEVHTKKTVMIKTIETRDGEVVSEATQQQHEVL

SEQ ID NO: 98 - protein, 284 aa, Gallus gallus
MEAIKKKMQMLKLDKENAIDRAEQAEADKKQAEDRCKQLEEEQQGLQKKLKGTEDEVEKYSESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESRVRQLEEELRTMDQSLKSLIASEEEYSTKEDKYEEEIKLLGEKLKEAETRAEFAERSVAKLEKTIDDLEESLASAKEENVGIHQVLDQTLLELNNL

SEQ ID NO: 99 - protein, 162 aa, Sus scrofa
MGLETEKADVQLFMDDDSYSRHSGVDYADPKKFVDPGTDRDPHRLNSNLKVGFEDVIAEPVSTHSFDKVWICSHALFEISKYVIYKFLTVFLAIPLAFAAGILFATLSCLHIWIIIPFVKTCLMVLPSVQTIWKSVTDVVIAPLCTSAGRSFSSVSLQLSHD

SEQ ID NO: 100 - protein, 1004 aa, Gallus gallus
MKMADAKQKRNEQLKRWIGSETDLEPPVVKRKKTKVKFDDGAVFLAACSSGDTEEVLRLLERGADINYANVDGLTALHQACIDDNVDMVKFLVENGANINQPDNEGWIPLHAAASCGYLDIAEYLISQGAHVGAVNSEGDTPLDIAEEEAMEELLQNEVNRQGVDIEAARKEEERIMLRDARQWLNSGHINDVRHAKSGGTALHVAAAKGYTEVLKLLIQARYDVNIKDYDGWTPLHAAAHWGKEEACRILVENLCDMEAVNKVGQTAFDVADEDILGYLEELQKKQNLLHSEKREKKSPLIESTANLDNNQTQKTFKNKETLIMEQEKNASSIESLEHEKADEEEEGKKDESSCSSEEEEDDDSESEAETDKAKTLANANTTSTQSASMTAPSVAGGQGTPTSPLKKFPTSTTKVSPKEEERKDESPASWRLGLRKTGSYGALAEITASKEAQKEKDSAGVIRSASSPRLSSSLDNKEKEKDGKGTRLAYVAPTIPRRLASTSDIDEKENRDSSASSIRSGSSYARRKWEEDVKKNSLNEGPTSLNTSYQRSGSFGRRQDDLVSSNVPSTASTVTSSAGLQKTLPASANTTTKSTTGSTSAGVQSSTSNRLWAEDSTEKEKDSVPTAVTVPVAPSVVNAAATTTAMTTATSGTVSSTSEVRERRRSYLTPVRDEESESQRKARSRQARQSRRSTQGVTLTDLQEAEKTIGRSRSTRTREQENEEKEKEEKEKQDKEKQEEKKESETKDDDYRQRYSRTVEEPYHRYRPTSTSTSTSSTSSLSTSTSSLSSSSQLNRPNSLIGITSAYSRSGTKESEREGGKKEEEKEEDKSQPKSIRERRRPREKRRSTGVSFWTQDSDENEQEHQSDSEEGTNKKETQSDSLSRYDTGSLSVSSGDRYDSAQGRSGSQSYLEDRKPYCSRLEKEDSTDFKKLYEQILAENEKLKAQLHDTNMELTDLKLQLEKTTQRQERFADRSLLEMEKRVSGKSQYLLGGKKSSRKKDI

SEQ ID NO: 101 - protein, 252 aa, Gallus gallus
MSNANFNRGPAYGLSAEVKNKLAQKYDPQTERQLRVWIEGATGRRIGDNFMDGLKDGVILCELINKLQPGSVQKVNDPVQNWHKLENIGNFLRAIKHYGVKPHDIFEANDLFENTNHTQVQSTLIALASQAKTKGNNVGLGVKYAEKQQRRFQPEKLREGRNIIGLQMGTNKFASQQGMTAYGTRRHLYDPKLGTDQPLDQATISLQMGTNKGASQGMTVYGLPRQVYDPKYCDAPGLLGEDGLNHSFYNSQ

SEQ ID NO: 102 - protein, 305 aa, Gallus gallus
MSSSQFNKGPSYGLSAEVKNRLAQKYDPQKEAELRTWIESVTGRQIGADFQKGLKDGVILCELMNKLQPNSVRKINRSALNWHQLENLSNFIKAMVSYGMNPVDLFEANDLFESGNLTQVQVSLLALAGMAKTKGLQSGVDIGVKYSERQQRNFDEAKMKAGQCVIGLQMGTNKCASQSGMTAYGTRRHLYDPKNQILPPMDHSTISLQMGTNKCASQVGMTAPGTRRHIYDAKMGLEKCDNSSMSLQMGSNQGANQSGQVFGLGRQIYDPKYCPQGTPGDAANAAGEPGADPPGYHYYHQEESC

SEQ ID NO: 103 - protein, 331 aa, Gallus gallus
MTHFNKGPSYGLSAEVKNKIALKYDPQIEEDLRNWIEEVTGLSIGANFQLGLKDGIILCELINKLQPGSVKKINQSKLNWHQLENIGNFIKAIQVYGMKPHDIFEANDLFENGNMTQVQTTLVALAGLAKTKGFHTTIDIGVKYAEKQARSFDAGKLKAGQSVIGLQMGTNKCASQAGMTAYGTRRHLYDPKMQTDKPFDQTTISLQMGTNKGASQAGMLAPGTRRDIYDQKHILQPVDNSTISLQMGTNKVASQKGMSVYGLGRQVYDPKYCAAPTEPVIHNGSQGTGTNGSEISDSDYQAEYPDDYHGEYQDDYQRDYHGQYSDQGIDY

SEQ ID NO: 104 - protein, 200 aa, Gallus gallus
MANKGPAYGMSRDVQSKIEKKYDDELEDRLVEWIVAQCGSSVGRPDRGRLGFQVWLKNGIVLSQLVNSLYPDGSKPVKIPDSPPTMVFKQMEQIAQFLKAAEDYGVVKTDMFQTVDLFEAKDMAAVQRTLVALGSLAVTKNDGHYHGDPNWFMKKAQEHKREFSESQLKEGKNIIGLQMGTNKGASQAGMSYGRPRQIIS

SEQ ID NO: 105 - protein, 748 aa, Gallus gallus
RLSYQRNDDDEEEAARERRRRARQERLRQKEEGDVSGEVTEKSEVNAQNSVAEEETKRSTDDEAALLERLARREERRQKRLQEALERQKEFDPTITDGSLSVPSRREVNNVEENETTGKEEKVETRQGRCEIEETETVTKSYQRNNWRQDGEEEGKKEEKDSEEEKPKEVPTEENQVDVAVEKSTDKEEVVETKTLAVNAENDTNAMLEGEQSITDAADKEKEEAEKEREKLEAEEKERLKAEEEKKAAEEKQKAEEEKKAAEERERAKAEEEKRAAEERERAKAEEERKAAEERERAKAEEERKAAEERAKAEEERKAAEERAKAEEERKAAEERAKAEKERKAAEERERAKAEEEKRAAEEKARLEAEKLKEKKKMEEKKAQEEKAQANLLRKQEEDKEAKVEAKKESLPEKLQPTSKKDQVKDNKDKEKAPKEEMKSVWDRKRGVPEQKAQNGERELTTPKLKSTENAFGRSNLKGAANAEAGSEKLKEKQQEAAVELDELKKRREERRKILEEEEQKKKQEEAERKIREEEEKKRMKEEIERRRAEAAEKRQKVPEDGVSEEKKPFKCFSPKGSSLKIEERAEFLNKSAQKSGMKPAHTTAVVSKIDSRLEQYTSAVVGNKAAKPAKPAASDLPVPAEGVRNIKSMWEKGNVFSSPGGTGTPNKETAGLKVGVSSRINEWLTKTPEGNKSPAPKPSDLRPGDVSGKRNLWEKQSVEKPAASSSKVTATGKKSETNGLRQFEKEP

SEQ ID NO: 106 - protein, 2541 aa, Gallus gallus
MVALSLKISIGNVVKTMQFEPSTMVYDACRMIRERVPEAQMGQPNDFGLFLSDEDPKKGIWLEAGKALDYYMLRNGDTMEYKKKQRPLKIRMLDGTVKTVMVDDSKTVTDMLMTICARIGITNYDEYSLVREIMEEKKEEVTGTLKKDKTLLRDEKKMEKLKQKLHTDDELNWLDHGRTLREQGIDDNETLLLRRKFFYSDQNVDSRDPVQLNLLYVQARDDILNGSHPVSFDKACEFAGYQCQIQFGPHNEQKHKPGFLELKDFLPKEYIKQKGERKIFMAHKNCGNMSEIEAKVRYVKLARSLKTYGVSFFLVKEKMKGKNKLVPRLLGITKECVMRVDEKTKEVIQEWSLTNIKRWAASPKSFTLDFGDYQDGYYSVQTTEGEQIAQLIAGYIDIILKKKKSKDHFGLEGDEESTMLEDSVSPKKSTVLQQQFNRVGKAELGSVALPAIMRTGAGGPENFQVGTMPQAQMQITSGQMHRGHMPPLTSAQQALTGTINSSMQAVNAAQATLDDFETLPPLGQDAASKAWRKNKMDESKHEIHSQVDAITAGTASVVNLTAGDPADTDYTAVGCAVTTISSNLTEMSKGVKLLAALMEDEGGNGRQLLQAAKNLASAVSDLLKTAQPASAEPRQNLLQAAGLVGQTSGELLQQIGESDTDPRFQDMLMQLAKAVASAAAALVLKAKNVAQKTEDSALQTQVIAAATQCALSTSQLVACTKVVAPTISSPVCQEQLIEAGKLVAKSAEGCVEASKAATNDDQLLKQVGVAATAVTQALNDLLQHIKQHATGGQPIGRYDQATDTILNVTENIFSSMGDAGEMVRQARILAQATSDLVNAIKADAEGETDLENSRKLLSAAKILADATAKMVEAAKGAAAHPDSEEQQQRLREAAEGLRMATNAAAQNAIKKKLVHKLEHAAKQAAASATQTIAAAQHAAASNKNPAAQQQLVQSCKVVADQIPMLVQGVRGSQSQPDSPSAQLALIAASQNFLQPGGKMVAAAKATVPTITDQASAMQLSQCAKNLAAALAELRTAAQKAQEACGPLEIDSALGLVQSLERDLKEAKAAARDGKLKPLPGETMEKCAQDLGNSTKAVTSAIAHLLGEVAQGNENYTGIAAREVAQALRSLSQAARGVAANSSDPQVQNAMLECASDVMDKANNLIEEARKAVAKPGDPDSQQRLVQVAKAVSQALNRCVNCLPGQRDVDAAIRMVGEASKRLLSDSFPPSNKTFQEAQSQLNRAAAGLNQSANELVQASRGTPQDLAKSSGKFGQDFNEFLQAGVEMASLSPTKEDQAQVVSNLKSISMSSSKLLLAAKALSADPTSPNLKSQLAAAARAVTDSINQLITMCTQQAPGQKECDNALRELETVKELLENPTQTVNDMSYFSCLDSVMENSKVLGESMAGISQNAKNSKLPEFGESISAASKALCGLTEAAAQAAYLVGVSDPNSQAGQQGLVDPTQFARANQAIQMACQNLVDPACTQSQVLSAATIVAKHTSALCNTCRLASSRTANPVAKRQFVQSAKEVANSTANLVKTIKALDGAFNEENRERCRAATAPLIEAVDNLTAFASNPEFATVPAQISPEGRRAMEPIVTSAKTMLESSAGLIQTARSLAVNPKDPPQWSVLAGHSRTVSDSIKKLITNMRDKAPGQRECDEAIDVLNRCMREVDQASLAAISQQLAPREGISQEALHNQMITAVQEINNLIEPVASAARAEASQLGHKVSQMAQYFEPLILAAIGAASKTPNHQQQMNLLDQTKTLAESALQMLYTAKEAGGNPKQAAHTQEALEEAVQMMKEAVEDLTTTLNEAASAAGVVGGMVDSITQAINQLDEGPMGEPEGTFVDYQTTMVKTAKAIAVTVQEMVTKSTTNPDELGILANQLTNDYGQLAQQAKPAALTAENEEIGSHIKRRVQELGHGCAALVTKAGALQCSPSDAYTKKELIESARKVSEKVSHVLAALQAGNRGTQACITAASAVSGIIADLDTTIMFATAGTLNRENSETFADHREGILKTAKALVEDTKVLVQNATASQEKLAQAAQSSVSTITRLAEVVKLGAASLGSEDPETQVVLINAVKDVAKALGDLIGATKAAAGKAGDDPAVYQLKNSAKVMVTNVTSLLKTVKAVEDEATKGTRALEATIEHIRQELAVFSSPVPPAQVSTPEDFIRMTKGITMATAKAVAAGNSCRQEDVIATANLSRRAIADMLRACKEAAYHPEVSADVRQRALRFGKECADGYLELLEHVLVILQKPTHELKQQLAGYSKRVASSVTELIQAAEAMKGTEWVDPEDPTVIAENELLGAAAAIEAAAKKLEQLKPRAKPKQADESLDFEEQILEAAKSIAAATSALVKAASAAQRELVAQGKVGVIPANAVDDGQWSQGLISAARMVAAATNNLCEAANAAVQGHASEEKLISSAKQVAASTAQLLVACKVKADHDSEAMKRLQAAGNAVKRASDNLVKAAQKAAAFQDHDETVVVKEKMVGGIAQIIAAQEEMLRKERELEEARKKLAMIRQQQYKFLPTELRDEEQN

SEQ ID NO: 107 - protein, 134 aa, Gallus gallus
MNCWNYYTDCILSDKYIDDVAIVGLSDNKYVWAAKPGGLLSAVSPREVDLITGQDRITFLTAGISIAGKKCIVIRDSLLVEGDNVMDIRSRGGDSRSICIGKTPKALIFLMGNRGVHGGVLNLKIHDMIAGMTL

SEQ ID NO: 108 - protein, 149 aa, Gallus gallus
MIQLQALLKESLIRTKHVENAAAIGINEREVCASTSGFYVPPENAINLIYAFYKNLLQVRKEGLYFRQKHYECVRADEHSIYLKNAEGGLIVVKTNALILIATYRVGMYPSVCVEAVEKLGKTNTCTCMAACQRFIGWLCSCKELQKCV

SEQ ID NO: 109 - protein, 423 aa, Gallus gallus
MATYSFRQTTSSVAGGPCGRSLRLGGGSFRAPSIHGGSGGRGVSVSSARFVSSGLGSGLGGGYGGAFSSSFSAGFGGGYGGGLGSGDGLLSGNEKTTMQNLNDRLASYLDKVRALEEANSDLETKIREWYLKQGPGPARDYSPYYKAIEDLRDQILAATIDNSKVVLQIDNARLAADDFKTKFETEQALRMSVEADINGLRRVLDELTLARTDLELQIENLKEELAYLKKNHEEEMSALGGQVASQVSVEVDSAPGIDLSKILADMRDQYEHMAEKNRKDAEAWFHSKTEELNRELAVNTEQLQSSKSEVTDLRRTLQGLEIELQSQLSMKGALESTLADTEGRYGAQLAQIQDMIGSIEAQLAELRADMERQNSEYKMLMDIKTRLEQEIATYRQLLEGQESQLFGSLSGSPDKRDKPADGK

SEQ ID NO: 110 - protein, 161 aa, Salmo salar
MDDVYKAAVENLTEEQKNEFKAAFDIACQGAEDGCISTKELGKVMRMLGQNPTPEELQEMIDEVDEDGSGTVDFDEFLVMMVRCMKEESKGKSEEELAELFRMFDKNGDGYIDLEELKTMLESTGEAITEDDIEELMKDGDKNNDGKIDYDEFLEFMKGVE

SEQ ID NO: 111 - protein, 161 aa, Gallus gallus
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKTEEELSDLFRMFDKNADGYIDLEELKIMLQATGETITEDDIEELMKDGDKNNDGRIDYDEFLEFMKGVE

SEQ ID NO: 112 - protein, 161 aa, Bos taurus
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKSEEELSDLFRMFDKNADGYIDLEELKIMLQATGETITEDDIEELMKDGDKNNDGRIDYDEFLEFMKGVE

SEQ ID NO: 113 - protein, 272 aa, Gallus gallus
MSDQQLDCALDLMRRLPPQQIEKNLSDLIDLVPSLCEDLLSSVDQPLKIARDKVVGKDYLLCDYNRDGDSYRSPWSNKYDPPLEDGAMPSARLRKLEVEANNAFDQYRDLYFEGGVSSVYLWDLDHGFAGVILIKKAGDGSKKIKGCWDSIHVVEVQEKSSGRTAHYKLTSTVMLWLQTNKTGSGTMNLGGSLTRQMEKDETVSDSSPHIANIGRLVEDMENKIRSTLNEIYFGKTKDIVNGLRSVQTFADKSKQEALKNDLVEALKRKQQS

SEQ ID NO: 114 - protein, 782 aa, Bos taurus
MSGAPKESLGPQGLPGLGKACLTTMDKKLNMLNEKVDKLLHFQEDVTEKLQCVYRGMGHLEQGLHRLEASRGLGPAGADRSPPSDAQAGWPEVLELVRAGRQDAAQQGARLEALFRMVMAVDKAIALVGAVLQNSKVVDFIMQGSVPWRKGSLADNKEQVEEKEAKPKHTLSTRGVQAELRGPWEESQKADLPEGTGSDLPTQTEAPPEQRDGISGPTQVRPEVEVQAPRASSKNPGIGLELSVVSERVSEAPTGQEAALSAGRGTSPSRPDPRPSVEGMRLTPAPPAQAKAAHGGGETPPRISIHVQETDTPGELLVTRGGSLRTSPPAETPAAVPPGEQDPPGPRCCPQAPGTESGKPILRGASVRKRSCDEGAKAKEKQGPGSELTMAPSRARRDKGADSGASGPQQDMNPGAGNPDPGKDCTAGGVGSAEAGSRTPPGAEASSLVLDDSPAPPAPFEHRVVSVRETSTSAGYTVCQHEVLGGGRFGQVHRCTEKATGLSLAAKIIKVKSAKDREDVKNEINIMNQLSHVNLIQLYDAFESKNSFTLVMEYVDGGELFDRITEEKYHLTELDVVLFTKQICEGVHYLHQHYVLHLDLKPENILCVNQTGHQIKIIDFGLARRYKPREKLKVNFGTPEFLAPEVVNYEFVSFPTDMWSVGVITYMLLSGLSPFLGETDAETMNFIVNCNWDFDADTFEGLSEEAKDFVSRLLVKEKSCRMSATQCLKHEWLNNLPAKASKSKVHLKSQLLLQKYMAQRKWKKHFYVVTAANRLRKFPTCP

SEQ ID NO: 115 - protein, 161 aa, Salmo salar
MNDIYKAAVEQLTDEQKNEFKAAFDIFVQDAEDGCISTKELGKVMRMLGQNPTPEELQEMIDEVDEDGSGTVDFDEFLVMMVRCMKDDSKGKTEEELADLFRMFDKNADGYIDLEELKVMLEATGEAITEDDIEELMKDGDKNNDGKIDYDEFLEFMKGVE

SEQ ID NO: 116 - protein, 167 aa, Sus scrofa
MATSELSCQVSEENCERREAFWAEWKDLTLSTRPEEGCSLHEEDAERRETYHQQGQCQALVQRSPWLVMRMGILGRGLQEYQLPYQRVLPLPIFTPAKVGAAKEEREETPIQLRELLALETALGGQCLDRQDVAEITKQLPPVVPVSKPGALRRSLSRSMSQEAQRG

SEQ ID NO: 117 - protein, 284 aa, Gallus gallus
MDAIKKKMQMLKLDKENALDRAEQAEADKKAAEERSKQLEDELVALQKKLKGTEDELDKYSESLKDAQEKLELADKKATDAESEVASLNRRIQLVEEELDRAQERLATALQKLEEAEKAADESERGMKVIENRAQKDEEKMEIQEIQLKEAKHIAEEADRKYEEVARKLVIIEGDLERAEERAELSESKCAELEEELKTVTNNLKSLEAQAEKYSQKEDKYEEEIKVLTDKLKEAETRAEFAERSVTKLEKSIDDLEDELYAQKLKYKAISEELDHALNDMTSI

SEQ ID NO: 118 - protein, 283 aa, Gallus gallus
MEAIKKKMQMLKLDKENAIDRAEQAEADKKQAEDRCKQLEEEQQGLQKKLKGTEDEVEKYSESVKEAQEKLEQAEKKATDAEAEVASLNRRIQLVEEELDRAQERLATALQKLEEAEKAADESERGMKVIENRAMKDEEKMELQEMQLKEAKHIAEEADRKYEEVARKLVVLEGELERSEERAEVAESKCGDLEEELKIVTNNLKSLEAQADKYSTKEDKYEEEIKLLGEKLKEAETRAEFAERSVAKLEKTIDDLEERSRQEAEKNRVLTNELRVILTELNN

SEQ ID NO: 119 - protein, 151 aa, Bos taurus
MMAEEHTDLEAQIVKDIHFKEIDLVNRDPKNINEDIVKVDFEDVIAEPVGTYSFDGVWKVSYTTFTVSKYWCYRLLSTLLGVPLALLWGFLFACISFCHIWAVVPCIKSYLIEIQCISHIYSLCIRTFCNPLFAALGQVCSNIKVMLRKEV

SEQ ID NO: 120 - protein, 1450 aa, Gallus gallus
MSSVAVPFYQRRHKHFDQSYRNIQTRYVLEEYAARKAASRQAAHYESTGLGKTTCRLCARRARSLAHEAMQESRKRTHEQKSHASDEKRIKFASELSSLEREIHMARHHAREQLDRLAIQRMVEENMALERHVVEEKISRAPEILVRLRSHTVWEKMSVRLCFTVQGFPSPVVQWYKNEELITPASDPAKYSVENKYGVHVLHINRADFDDSATYSAVATNIHGQASTNCAVVVRRFRESEEPHPAGIMPFHLPLSYDVCFTHFDVQFLEKFGVTFATEGETLTLKCSVLVTPELKRLRPRAEWYRDDVLIKDSKWTKLYFGEGQAALSFTHLNKDDEGLYTLRMVTKGGVNECSAFLFVRDADALIAGAPGAPMDVKCHDANRDYVIVTWKPPNTTSQNPVIGYFVDKCEVGLENWVQCNDAPVKICKYPVTGLYEGRSYIFRVRAVNSAGISRPSRVSEPVAALDPVDLERTQTVHVDEGRKIVISKDDLEGDIQIPGPPTNVHASEISKTYVVLSWDPPVPRGREPLTYFIEKSMVGSGSWQRVNAQVAVKSPRYAVFDLAEGKPYVFRVLSANKHGISDPSEITEPIQPQDIVVVPSAPGRVVATRNTKTSVVVQWDKPKHEENLYGYYIDYSVVGSNQWEPANHKPINYNRFVVHGLETGEQYIFRVKAVNAVGFSENSQESEAIKVQAALTCPSYPHGITLLNCDGHSMTLGWKAPKYSGGSPILGYYIDKREANHKNWHEVNSSVISRTIYTVEDLTEDAFYEFKIAAANVVGIGHPSDPSEHFKCKAWTMPEPGPAYDLTVCEVRNTSLVLLWKAPVYEGKSPITGYLVDYKEVDTEDWITANEKPTSHRYFKVTDLHQGHTYVFKVRAVNDAGVGKSSEISEPVFVEASPGTKEIFSGVDEEGNIYLGFECKEATDASHFLWGKSYEEIEDSDKFKIETKGDHSKLYFKHPDKSDLGTYCISVSDTDGVSSSFVLDEEELERLMTLSNEIKNPTIPLKSELAYEVLDKGEVRFWIQAESLSPNSTYRFVINDKEVENGDRHKISCDHSNGIIEMVMDKFTIDNEGTYTVQIQDGKAKNQSSLVLIGDAFKAILAESELQRKEFLRKQGPHFSEFLYWEVTEECEVLLACKIANTKKETVFKWYRNGSGIDVDEAPDLQKGECHLTVPKLSRKDEGVYKATLSDDRGHDVSTLELSGKVYNDIILALSRVSGKTASPLKILCTEEGIRLQCFLKYYNEEMKVTWSHRESKISSGEKMKIGGGEDVAWLQITEPTEKDKGNYTFEIFSDKESFKRTLDLSGQAFDDALTEFQRLKAAAFAEKNRGKVIGGLPDVVTIMDGKTLNLTCTVFGNPDPEVVWFKNDKALELNEHYLVSLEQGKYASLTIKGVTSEDSGKYSIYVKNKYGGETVDVTVSVYRHGEKIPEVNQGQLAKPRLIPPSSST

SEQ ID NO: 121 - protein, 2723 aa, Bos taurus
MMNNSGYSEAPGFGLGDEVDDMPSTEKDLAEDAPWKKIQQNTFTRWCNEHLKCVGKRLTDLQRDLSDGLRLIALLEVLSQKRMYRKFHPRPNFRQMKLENVSVALEFLEREHIKLVSIDSKAIVDGNLKLILGLIWTLILHYSISMPMWEDEDDEDARKQTPKQRLLGWIQNKVPQLPITNFNRDWQDGKALGALVDNCAPGLCPDWEAWDPNQPVENAREAMQQADDWLGVPQVIAPEEIVDPNVDEHSVMTYLSQFPKAKLKPGAPVRSKQLNPKKAIAYGPGIEPQGNTVLQPAHFTVQTVDAGIGEVLVYIEDPEGHTEEAKVVPNNDKNRTYAVSYVPKVAGLHKVTVLFAGQNIERSPFEVNVGMALGDANKVSARGPGLEPVGNVANKPTYFDIYTAGAGTGDVAVVIVDPQGRRDTVEVALEDKGDSTFRCTYRPVMEGPHTVHVAFAGAPITRSPFPVHVAEACNPNACRASGRGLQPKGVRVKEVADFKVFTKGAGSGELKVTVKGPRGTEEPVKVREAGDGVFECEYYPVVPGKYVVTITWGGYAIPRSPFEVQVSPEAGIQKVRAWGPGLETGQVGKSADFVVEAIGTEVGTLGFSIEGPSQAKIECDDKGDGSCDVRYWPTEPGEYAVHVICDDEDIRDSPFIAHIQPAPPDCFPDKVKAFGPGLEPTGCIVDKPAEFTIDARAAGKGDLKLYAQDADGCPIDIKVIPNGDGTFRCSYVPTKPIKHTIIVSWGGVNVPKSPFRVNVGEGSHPERVKVYGPGVEKTGLKANEPTYFTVDCSEAGQGDVSIGIKCAPGVVGPAEADIDFDIIKNDNDTFTVKYTPPGAGRYTIMVLFANQEIPASPFHIKVDPSHDASKVKAEGPGLNRTGVEVGKPTHFTVLTKGAGKAKLDVHFAGAAKGEAVRDFEIIDNHDYSYTVKYTAVQQGNMAVTVTYGGDPVPKSPFVVNVAPPLDLSKVKVQGLNSKVAVGQEQAFSVNTRGAGGQGQLDVRMTSPSRRPIPCKLEPGGGAETQAVRYMPPEEGPYKVDITYDGHPVPGSPFAVEGVLPPDPSKVCAYGPGLKGGLVGTPAPFSIDTKGAGTGGLGLTVEGPCEAKIECQDNGDGSCAVSYLPTEPGEYTINILFAEAHIPGSPFKATIRPVFDPSKVRASGPGLERGKAGEAATFTVDCSEAGEAELTIEILSDAGVKAEVLIHNNADGTYHITYSPAFPGTYTITIKYGGHPVPKFPTRVHVQPAVDTSGVKVSGPGVEPHGVLREVTTEFTVDARSLTATGGNHVTARVLNPSGAKTDTYVTDNGDGTYRVQYTAYEEGAHLVEVLYDDVAVPKSPFRVGVTEGCDPTRVRAFGPGLEGGLVNKANRFTVETRGAGTGGLGLAIEGPSEAKMSCKDNKDGSCTVEYIPFTPGDYDVNITFGGRPIPGSPFRVPVKDVVDPGKVKCSGPGLGAGVRARVPQTFTVDCSQAGRAPLQVAVLGPTGVAEPVEVRDNGDGTHTVHYTPATDGPYTVAVKYADQEVPRSLSSPFKIKVLPAHDASKVRASGPGLNASGIPASLPVEFTIDARDAGEGLLTVQILDPEGKPKKANIRDNGDGTYTVSYLPDMSGRYTITIKYGGDEIPYSPFRIHALPTGDASKCLVTVSIGGHGLGACLGPRIQIGEETVITVDAKAAGKGKVTCTVSTPDGAELDVDVVENHDGTFDIYYTAPEPGKYVITIRFGGEHIPNSPFHVLACEAMPRVEEPPDVPQLHRPSAYPTHWATEEPVVPAEPMESMLRPFNLVIPFTVQKGELTGEVRMPSGKTARPNITDNKDGTITVRYAPTEKGLHQMGIKYDGNHIPGSPLQFYVDAINSRHVSAYGPGLSHGMVNKPATFTIVTKDAGEGGLSLAVEGPSKAEITCKDNKDGTCTVSYLPTAPGDYSIIVRFDDKHIPGSPFTAKITGDDSMRTSQLNVGTSTDVSLKITESDLSQLTASIRAPSGNEEPCLLKRLPNRHIGISFTPKEVGEHVVSVRKSGKHVTNSPFKILVGPSEIGDASKVRVWGKGLSEGHTFQVAEFIVDTRNAGYGGLGLSIEGPSKVDINCEDMEDGTCKVTYCPTEPGTYIINIKFADKHVPGSPFTVKVTGEGRMKESITRRRQAPSIATIGSTCDLNLKIPGNWFQMVSAQERLTRTFTRSSHTYTRTERTEISKTRGGETKREVRVEESTQVGGDPFPAVFGDFLGRERLGSFGSITRQQEGEASSQDMTAQVTSPSGKTEAAEIVEGEDSAYSVRFVPQEMGPHTVTVKYRGQHVPGSPFQFTVGPLGEGGAHKVRAGGTGLERGVAGVPAEFSIWTREAGAGGLSIAVEGPSKAEIAFEDRKDGSCGVSYVVQEPGDYEVSIKFNDEHIPDSPFVVPVASLSDDARRLTVTSLQETGLKVNQPASFAVQLNGARGVIDARVHTPSGAVEECYVSELDSDKHTIRFIPHENGVHSIDVKFNGAHIPGSPFKIRVGEQSQAGDPGLVSAYGPGLEGGTTGVSSEFIVNTLNAGSGALSVTIDGPSKVQLDCRECPEGHVVTYTPMAPGNYLIAIKYGGPQHIVGSPFKAKVTGPRLSGGHSLHETSTVLVETVTKSSSSRGSSYSSIPKFSSDASKVVTRGPGLSQAFVGQKNSFTVDCSKAGTNMMMVGVHGPKTPCEEVYVKHMGNRVYNVTYTVKEKGDYILIVKWGDESVPGSPFKVNVP

SEQ ID NO: 122 - protein, 352 aa, Gallus gallus
MTLPFRKDLDKYKDLDEDDILGKLSEEELKQLETVLDDLDPENALLPAGFRQKDQTAKKASGPFDRERLLAYLEKQALEHKDREDYVPFTKEKKGKVFIPKQKPAQSYAEEKIALDPELEEALTSATDTELCDLAAILGMSNLITNNQFCDIVGSSNGVGKDSFSNIVKGEKMLPVFDEPPNPTNVEETLQRIKDNDSRLVEVNLNNIKNIPIPTLKEFAKALETNTHVKNFSLAATRSNDPVAVALADMLRVNTKLKSLNIESNFITGVGILALVDALKDNETLTEIKIDNQRQQLGTLAEVEIAKMLEENTKILKFGYHFTQQGPRARAAAAITKNNDLVRKRRVEGDGQ

SEQ ID NO: 123 - protein, 359 aa, Gallus gallus
MSYRKELEKYRDLDEDKILGALTEEELRKLENELEELDPDNALLPAGLRQRDQTQKPPTGPFKREELMAHLEQQAKDIKDREDLVPFTGEKRGKAWIPKQKPMDPVLESVTLEPELEEALANASDAELCDIAAILGMHTLMSNQQYYEALGSSTIVNKEGLNSVIKPTKYKPVPDEEPNSTDVEETLKRIQNNDPDLEEVNLNNIMNIPVPTLKALAEALKTNTYVKKFSIVGTRSNDPVAFALAEMLKVNNTLKSLNVESNFISGSGILALVEALQSNTSLIELRIDNQSQPLGNNVEMEIANMLEKNTTLLKFGYHFTQQGPRLRASNAMMNNNDLVRKRRLAELNGPIFPKCRTGV

SEQ ID NO: 124 - protein, 359 aa, Sus scrofa
MSYRRELEKYRDLDEDEILGALTEEELRTLENELDELDPDNALLPAGLRQKDQTTKAPTGPFKREELLDHLEKQAKEFKDREDLVPYTGEKRGKVWVPKQKPMDPVLETVTLEPELEEALANASDAELCDIAAILGMHTLMSNQQYYQALGSSSIVNKEGLNSVIKPTQYKPVPDEEPNATDVEETLERIKNNDPKLEEVNLNNIRNIPIPTLKAYAEALKENSYVKKFSIVGTRSNDPVAFALAEMLKVNKVLKTLNVESNFISGAGILRLVEALPYNTSLVELKIDNQSQPLGNKVEMEIVSMLEKNATLLKFGYHFTQQGPRLRASNAMMNNNDLVRKRRLADLTGPIIPKCRSGI

SEQ ID NO: 125 - protein, 471 aa, Gallus gallus
MSRRVVRQSKFRHVFGQPVKADQMYEDIRVSKVTWDSSFCAVNPKFVAIIVEAGGGGAFMVLPLAKTGRVDKNHPLVTGHTAPVLDIDWCPHNDNVIASASEDTTVMVWQIPDYVPVRSITEPVVTLEGHSKRVGIICWHPTARNVLLSAGCDNLVILWNVGTGEMLLALEDMHTDLIYNVGWNRNGSLLVTTCKDKKVRVIDPRKQTVVAEITKPHDGARPIRAIFMADGKIFTTGFSKMSERQLGLWDLKNFEEPIALQEMDTSNGVLLPFYDPDTNIVYLCGKGDSSIRYFEITDEAPYVHYLNTYSSKEPQRGMGFMPKRGLDVSKCEIARFFKLHERKCEPIVMTVPRKSDLFQDDLYPDTPGPEPALEADEWLSGKDAEPILISLRDGYVPVKNRELKVVKKNILDSKPPPGPRRSHSTSNTDISTPALDEVLEEIRVLKETVQAQEKRISALEHKLCQFTNGTD

SEQ ID NO: 126 - protein, 962 aa, Gallus gallus
MYFSFMLAGLHRRKEFSLLFPPSIKMRVSVTQEFTEDENENGEEERVFLKPVIEDRNMELARKCSEIISDVHYKEEFEKSKGKCIFVPDTPQLKHVKSVGAFISEVKYKGAAKKDLSNSLYQQMPATIDSAFAKELTQLQSKVLYKQKHDAEKGTSDYAHMKEPPDIKHAMEVNKYQSDVSYKRDVQDTHRYTEVLNRPDIKMATEITKIISDAEYKKGRGEMNKEPAVLGRPDFEHAKGVSKLLSQVKYKEQFKKEMKSHQYNPLDSASFKQAQIASTLASNVNYKKDYKESLHDPASDLPNLLYLNHALNISKMHSDVKYRENYEKSKGKSMLEFVDTPLYQVSKDVQKMQSEKIYRKDFEESLKGRPSLDLDKTPEFLHIKQVTNLLKEKEYRKDLEEWMKGKGMTVFEDTPDLIRVKNAAQILNEKQYKKDLETEIKGKGMQVGPDTPEIRRAKKASEIASTKEYKKDLENEIKGKGMEVGMDTPDIQRAKKASEIVSQKEYRKDLETEIIGKGMQVGPFTPEIQRVKRASEIASQKMYKDEAEKMLCNYSAVPDTPEMERIKSTQKNISSVFYKKVVGAGTAVKETPEIERVKKNQQNISSIKYKEETQHATPISDPPELRRIKENQKNISNVHYKEQLCRATPVSVTPEIERVKRNQENVSMVHYREQPGKATAVSITPEIERVKKNQDNISSVKYSSDQRQMKGRRSVILDTPELRHVKETQNNISMVKYHEDFEKTKGRGFTPVVDDPITERVRKNTQVVSDAAYKGVHPHIVEMDRRPGIIVDLKVWRTDPGSIFDIDPLEDNIQSRSLHMLSERASRYSKQYLHSTSLGDYKSDGSDTNPTFSYCSEITRPSDEGAPVLPGAYQQSQSQGYGYMHQTSMSSMRSVHSQPHPAGLRTYRAMYDYSAQDEDEVSFRDGDYIINVQPIDDGWMYGTVQRTGKTGMLPANYIEFVN

SEQ ID NO: 127 - protein, 1730 aa, Bos taurus
MNVQACSRCGYGVYPAEKINCLDQIWHKACFHCEVCKMMLSVNNFVSYQKKPYCHAHNPKNNTFTSVYYTPLNLNVRKPPEAICGIGGQEDGERFKSVFHWDMKSKDEAAAPNRQPQVDERAYWSGYREGDAWCPGALPDPEIVRMVEARKSLGEEYPEDYEQQRGKGSFPAMITPAYQRAKIANQLASQVEYKRGHDERISRFSTVADTPELLRAKAGGQLQSDVRYTEAYEQQRGKGSFPAMITPAYQIAKRANELASDVRYHQQYQREMKGMAGPAAGAEGPLPKEYMDQYGQGYSEEYGEHRGKGSFPAMITPAYQNAKKANELASDIKYRQDFNKMKGAAHYHSLPAQDNLVLKRAQSVNKLVSEVEYKKDLESSKGHSINYCETPQFRNVCKISKFTSDNKYKENYQNRMRGRYEGVGMDKRMLHALKVGSLASNIAYKADYKHDVVDYNYPATLTPSYQTTVKLAPLKDVNYRQSIDKLKYSSVTNTPQIVQAKINAQQLSHVNYRADYEKNKLNYTLPQDVPQLVKARTNAELFSEVKYREGWEKTKGKGFEMKLDAMSLLAAKASGELASNIKYKEEYEKAKGKVLGTTDSRLLHSLQVAKMSSEVEYKKGFEKSKTHFHLPMDMVNIRHAKKAQALASDLDYRKRLHEYTVLPEDMKTLWAKKAYGLQSELQYKADLAWMKGVRWLTEGSLNLEQAKKAGQLVSEKNYRQRVDELKFTSVADSSQMEHAKKSQELQSGVAYKAEHEQSVHQYSISKDEPLFLQARANAANLSEKLYKSSWENQKAKAFDLRLDSLAFLAAKAKRDLASEVKYKEDYEKSRGKLIGAQGAQGDSQMSHSLQMSKLQSELEYKKGFEDTKSQCHVPLDMIHLVHARKAQHLATDIGYKTASHHFTALPTDMKVEWAKKAYGLQSDNQYRADVKWMKGTGWVATGSLNVEQAKKAGELISEKKYRQHPDALKFTSIKDTPEMVQARISYTQAVDRLYREQGENLKHHYTQTTDLPEVLLAKLNAMNISETRYKESWSKLRDGGYKLRLDAIPFQAAKASGEIISDYKYKEAFEKMKGQMLGSRSLEDDISLAHSVYASSLQSQVNYKKDFEHSKAQFHLPLDMVTLVHAKKAQTLASDQDYRHPLPQYTSLAEDLRLSCAKRAHKLQSENLYRSDLNFMRGVACVIPGTLEIEGRKRASELISESKYRQHPQSLKYTAVTDTPSLTHAKLSNQITNERLYKAAGEDARHQYTMTLGLPEFVRAKTNAANLSDAKYKESWRNLHAQGYKLTIDALPFQAARVSGDIASDFLYRHDFVKERGRLIGAQSVSDDPRLQHCQRVGQLQSELQYRRQAAGSRAQCHLPMDMVPLVHARKAQALASDLDYRTQCHAFTALPEDLRMAWAKKAHALQSELRYKSDLMGMKGTGWLALSSPQIESAKKAGELISETKYREKPDTIKFTTVVDSPDLVHAKNSYMHCNERLYRSGDAESRHRYTLVPDHPDFTRARLNALNLSDKVYRHSWEQTRAGGYDFRLDAIPFQTARASREIASDFRYKEAFLRDRGLQIGYRSINDDPRTKHFLSVGRLQSDNEYKKDFAKSRSQFHSRPDQPGFLQAKRSQQLASDVHYRQPLPQPTCDPEQLGLKHAQKAHRLQSDVKYKSDLNLTRGIGWTPPGSYKVEMARRAAELANARGLGLQGAYGGPEAVEPRDDQSGFVNPDATEILHVKRRKAPLF

SEQ ID NO: 128 - protein, 1736 aa, Gallus gallus
MNVQPCARCGYGVYPAEKINCIDQTWHKACFHCEVCKMMLTVNNFVSHEKKPYCQVHNPKNNAFTSIFETPINLNAKKLSEVVSEVKYREESEQFKSAFQWDVRSRDIEAAYKAQHLSSQNAYYAAYEGGTSWYSGNMPDPQMVTVTQAQKNLNDLQYTEEYELRQGKGSFPAMITPGYQVAKRATQLSSNVEYRRGHEERVSKFTSVVDTPDILHAKAGGQLASDLKYTEDFEEQRGKGSFPAMITPAYQIAKRANELASDVKYHQTYEKEIKGKASHTAGTDVTFTRENVDQYGQDYMNEYEERRGKGSFPAMITPAYQNAKKANELASDIKYKKDLSKMKGAAHFHSLTAEDNLVLKQAQSANKLVSEVEYKKDLGNNRGYSVNYCDTPQFKNVSKISKYTSDIKYKETYQNQMKGHYMGIGMDKRMLHAMKVGNLASNIAYKSDYKHDGVDYNYPATLTPSYQTTRKLVPLKDVNYRQSIDKMKYSSVASTPEIAQAKINAQQLSDLNYRAQYEKTKTNYTLPQDIPQLVKAKANAELYSEVKYKEGWEKSKGQGFEMKLDSLPLLAAKASRDLASDIKYKEEYEKTKGKAIETKDSRLLHSLQVAKMSSEIAYKKDFEESKTHFHLPMDMVNLRHAKKAQALASDLDYRKRLHEYTVVPEDLKTKWAKKAYGLQSDLQYREDLMWMKGVGCITEGSLNIQQAKKAGDLVSEKKYRQKVDALKFTSVADSSHIKHAKKSQELQSDVAYRSGKEQFLHQYTITKDDPVFLLAKANAANISEKLYRSSWEKQKEKGFVLRLDALSFLTAKAKRDLASDIKYKEGYEKMKGKLIGVKAVEEDSKMAHSLQMSKLQSDLEYKKAFEDTKNQFQVSMDMANLVHAKKAQNLASDKGYRTALHHYTVLPTDRKVAWAKWAYGLQSDNRYRADLNWMKGAGWIATGSLNVEQAKKAGELISEKKYRQHPYALKFTSIKDTPEMIQARISYNQAVDRLYKEHGESIKHQYTLTADLPEILRAKLNTMNISEIRYKESWQKMKDGGYQLRLDAIPFQAAKASGEIISDHKYKEAFEKMKGQMIGSCGLDGDIRIAHSVHASSLQSDVKYKQGFEDTKTHFHLPLDMINLVHARKAQSLVSEQEYRQLLHQYTSLTDDLRLQCAKNAYKLQSENLYRSDMNFMRGVGCITPGALEIEGKKKASELISESKYRQQPHSFKYTSVTDSPGLLHAKFSNMIANERLYKAAGEDIQHHYTPTLGLPEFTQARINAANLSDVKYRESWHNLCAQGYKLTMEALPFQTARASREIASDYQYKHNFVMERGKHIGARSVLDDTRLLHCLHAAKLQSEQEYKKGSQGVWSQYYLPMDMVNLVHARKAQALASDQEYRKRLHEFTALPEDLKMKWAKRAHMLQSEHRYKSDLNFMKGVGWMALRSPQIESVKKAGELISETKYRQKPESLKFTAVVDSPDLIHAKNSYLQCNDRLYKAGDSEARHRYTLPPDHPDFIRARQNALNISDKVYKTSWEQTRASGYDFRIDAIPFQTAKASREIASDYKYREAFLRDRGQRIGFFSANDDAHTRHVLRVGKLQSDNLYRSGYAQNRGHFQSHLNQPGFLHAKRSQQLASNVNYKQPLHQYTCDPEQLNVKHAKQAYKLQSDVKYKSDLNWLRGIGWTPPGSYKVERARRAAELAYLREMGLQAASAQYGPEADQVGPEGSQQVTVNPDASEILQVKRRKMQLYK

SEQ ID NO: 129 - protein, 399 aa, Bos taurus
MTSYSYRQSSSTSSFGGMGGGSMRFGAGGAFRAPSIHGGSGGRGVSVSSARFVSSSSGGYGGGYGGALATSDGLLAGNEKLTMQNLNDRLASYLEKVRALEEANGDLEVKIRDWYQKQGPGPARDYSHYFKTIEDLRDQILGATIENSKIVLQIDNARLAADDFRTKFETEQALRMSVEADINGLRRVLDELTLARTDLEMQIEGLKEELAYLKKNHEEEMSVLKGQVGGQVSVEVDSAPGIDLAKILSDMRSQYEVIAEKNRKDAEAWFISQTEELNREVAGHTEQLQISKTEVTDLRRTLQGLEIELQSQLSMKAALEGTLAETEARFGAQLAQIQALISGIEAQLSDVRADTERQNQEYQHLMDIKTRLEQEIATYRNLLEGQDAYFNDLSLAKAL

SEQ ID NO: 130 - protein, 1467 aa, Bos taurus
MSLVAVPFYQKRHKHFDQSYRNIQTRYLLDEYSAKKKRASAQSSLQRSVTRKSSSQRESSRAALGRTTCRLCAKRMSSALEEEAQERKRRYQSMVAAYGEAKRERYLSELAQLEEDVHVARTHARDQLDRLALQHAVDDRLAWERHSFEERISRAPEILVRLRSHTVWERMSVKLCFTVQGFPTPVVQWYKDGSLICQAGEPGKYRIDSKYGVHTLEINRADFDDTATYSAVATNVHGQVSTNAAVVVRRFRGDEEPFHSVGLPIGLPLSSVIPYTHFDVQFIEKFGVTFRREGETLTLKCTLLVTPDLKRVQPRAEWYRDDVLLKESKWTKMFFGEGQASLSFSHLNKDDEGLYTLRIVSRGGITDHSAFLFVRDADPLVTGAPGAPMDVHCHDVNRDYVIVTWKPPNATKESPVIGYFIDRCEVGTDNWIQCNDAPVKICKYPVTGLFEGRSYIFRVRAVNSAGISRPSRVSEAVAALDPVDLRRLQAVHLEGEKDIVVYQEELEGEVQIPGPPTNVHASETSRTYVVLSWDPPEPRGKEPLMYFIEKSMVGSGSWQRVNAQTAVRSPRYAVFDLAEGKPYVFRVLSANKHGLSDPSEITAPIQAQDTIVVPSAPGRVLASRNTRTSVVVQWDRPKHEEDLLGYYVDCSVAGSNVWEPCNHKPIGYNRFVVHGLTTGEQYIFRVKAVNAVGTSENSQESDVIKVQAALTVPSHPYGITLLNCDGHSMILGWKVPKFSGGSPILGYYVDKREAHHKNWHEVNSSPLKERILTVEGLTEGSLYEFKIAAANMAGIGQPSDPSELFKCEAWTMPEPGPAYDLTFCEVRDTSLVLLWKPPVYSGSSPVSGYFVDYKEEDSGEWLTVHETATPHRYLKVCDLHQGKTYVFRVRAVNASGVGRPSDTSEPVLVEARPGTKEVSAGVDEEGNVYLSFDCPEVTDASHFTWCKSYEDIADDDRFKVETAGDHSKLYFKNLDKEDLGTYSVSVSDTDGVSSSFVLDEEELERLKALSNEIKNPTIPLKSELAYEIFDKGQVRFWLQAEHLSPDSSYRFIINDREVADSETHRIKCDKSTGIIEMVMDRFTIDNEGTYTVQIQDGKAKSQSSLVLIGDAFKAVLKEAEFQRKEFLRKQGPHFAEYLHWDVTEECEVRLVCKVANTKKETVFKWLKDDVLYETEKMPDLEKGVCELLIPKLSKKDHGEYKATLKDDRGQDVSTLEIAGKVYEDMILAMSRVCGASASPLKILCTPEGIRLQCFMKYFTEEMKVNWYHKDAKITSSEHMRIGGNEQMAWLQICEPTEKDKGKYTFEILDGKKNHQRSLDLSGQAFDEAFAEFQQLKAAAFAEKNRGKVIGGLPDVVTIMEGKTLNLTCTVFGNPDPEVVWFKNDKDIELSDHFSVKVEQGKYVSMTIKGVTSEDSGKYSIHVKNKYGGEKIDVTVSVYKHGEEIPDVVLPQQAKPKLLPAAAPSPAH

SEQ ID NO: 131 - protein, 1320 aa, Bos taurus
MQDDSLEASTSISQLLRESYLAETRHRGNNERSRAEPSSNPFHFGGSSGAAEGGGGQDDLPDLSAFLSQEELDESVNLARLAINYDPLEKADEAQARKRFSSDQTKHSSISSFDPNFCQDNSQSPTNSKESLQETKRPQYSSEAQSKKVFLNKAADFIEELSSLFKAHSSKRIRPRACKNHKSKLESQNQVMQENSSSFPDLSERRERSSVPIPIPADTRDNEVNHALEQQEAKRREAEQAAGEAASGDTTPGSSPSSLYYEEPLGQPPRFTQKLRSREVPEGTRVQLDCIVVGIPPPQVRWYCEGKELENSPDIHIIQAGNLHSLTIAEAFEEDTGRYSCFASNIYGTDSTSAEIYIEGVSSSDSEGDPNKDEMNRIQKPNEVSSPPTTSAGIPSAVPQTQHVVVQPRVSTIQQCQSPTNYLQGLDGKPIIAAPVFTKMLQNLSASEGQLVVFECRVKGAPSPKVEWYREGTLIEDSPDFRILQKKPRSMAEPEEICTLVIAEVFAEDSGCFTCTASNKYGTVSSIAQLDVRGNEDLRNNGSLHSANSTTNLALTEQQPSPPNPEPPVEQPPKPKLEGVLVNHNEPRSSSRIGLRVHFNLPEDDKGSEESSEGGVVTTHQTRPDSFQERFNGQSAKISEPSSPVKEPPPVLAKPKLDSSQLQQLHNQVLLEQHHLQNPSPSSPKEFPFNMSVLNSTTVPAVTVSSKHAKAPPSQTFSLARPKHFFPSTSTTVASVSPSSSPVFTLSSTPQAMQRTMSKESLLVTHPSAQTKSLGGASIQNEPLPTPAPTEPAQLPLTFSISSGNQFQPRCVSPAPVSPTGRIQNPVAFLSSVLPSLPAIPPTNAMGLPKSAPSMPSQGLMKKNTKSSHPVSDDYIRETKNAVILDLGKKMNFSDVRSNQQEYKISSFEQRLMNEIEFRLERTPVDESDDEIQHDEIPTGKCIAPIFDKRLKHFRVTEGSPVTFTCKIVGIPVPKVYWFKDGKQISKRNEHFKMKREGDGTCSLHINSTSSDDDGNYTIMAANPQGRISCSGHLMVQGLPIRSRLTAAGQSHRGRSRVQERDKEPLQERFFRPHFLQAPGDMVAHEGRLCRLDCKVSGLPPPELTWLLNGQPVLPDTSHKMLVRETGVHSLLIDPLTQRDAGTYTCIATNKTGQNSFSLELTVVAKEVKKAPVILEKLQNSGVPEGHPVRLECRVIGMPPPVFYWKKDNETIPFTRERISMHQDTTGYVCLLIQPAKKSDAGWYTLSAKNEAGIVSCTARLDIYAQWHHQIPPPMTVRPSGSRYGSLTSKGLDIFSAFSSMESTMVYSCSSRSVVESDEL

SEQ ID NO: 132 - protein, 264 aa, Bos taurus
MLSHNTMVKQRKQQASAIMKEIHGNDVDVMHLGKKVSIPRDIMLEELSHLSNRGARLFKMRQRRSDKYTFENFQYETKAQINHNIAMQNEKLDGINLESGSQQAPFTPPNTPDPRSPPNPENIAPGYSGPLKEIPPERFNTTAVPKYYQSPWEQAISNDPELLEALYPKFFKPEGKAELPDYRSFNRVATPFGGFEKASKMVKFKVPDFDLLLLTDPRFMAFANPLSGRRSFNRTPKGWISENIPIVITTEPTEDNTIPESEDL

SEQ ID NO: 133 - protein, 263 aa, Gallus gallus
MLSHSAMVKERKQQASAIMDEIQRNVSPSLNLGKKVSTPRDIMVEELSTLSNRGARLFKRRQRRSDKYTYENYHYVANKPRNRGEPIHSVKMDGLSVEGIPQHAPMTPPNTPDPRSPPHPDNIAPGYSGPLKEIPPEKFNTTSVPKYYQSPWIEAIRDDPELLEALYPKLFKPEAKPELPDYRSFNRVATPFGGFERASKLVKFKVPDYNLLMLNDPRFMILANPLATRRSFNRTPKGWTSENIPVVFIQPSDSNNVPETEDL

SEQ ID NO: 134 - protein, 264 aa, Sus scrofa
MLSHNTMVKQRKQQASAIMKEIHGNDVDGMDLGKKVSIPRDIMLEELSHLSNRGARLFKMRQRRSDKYTFENFQYESKAQINHNIAMQNGKLDGNNLESGSQQAPFTPPNTPDPRSPPNPENIAPGYSGPLKEIPPERFNTTAVPKYYQSPWEQAISNDPELLEALYPKLFKPEGKAELPDYRSFNRVATPFGGFEKASKIVKFKVPDFELLLLTDPRFMAFANPLSGRRSFNRTPKGWISENIPIVITTEPTEDTTIPESDDL

SEQ ID NO: 135 - protein, 479 aa, Bos taurus
MASPEPRRGGNGSAQAARATRPEVISPLQEENSASLYELGTWNPEVTLSLEGTLNLEDILYLGDTGDLDEALYVEETEPPEETLHIEETRMPDEALYLEEPVRPEAALYVEEPVKLEGVLYVEEPVKTASPEQIVHGGDRVLSEAKSKPKESLQAGPSPSTEGSLSIEDLELLEGRFQQCIEAVAQLEEERDQLIHELVLLREPALQEVQQVHRDILAAYKQHAQAELERDGLREEIRLVKQKLFKVTKECVAYQYQLECRRQDVAQFADFREALTTRAAQLSEELTQLREAYQKQKEQLRQQLEAPQSQRDGHFLQESRRLSAQFESLMAESRQGLEEEYEPQLLRLLERKEAAAKALQKTQAEIQEMKEALRPLQAEAQKLHLQNRNLEDQITLVRQKRDEEVQQYREQLEEMEERQRQLRSGVQLQQQKNKEMEQLRVSLAEELATYKAMLPKSLEQANAPTSEAGGIETPSQGAV

SEQ ID NO: 136 - protein, 323 aa, Gallus gallus
MDAEGAGTPQAPEGTERPCLSLEELGEYFQECIEAVEQLEKERDALIEELTQLREPALQDIRHAHQEIQAACRLLAKVELERDNLRDEIRQIKQKLFKVTKECVACQYQLESRRHDLSQHAAYRDELESQAGRLTGELSRLRESCEKEKEALRQRLEAPPCRQDPQYLQESRRLSAEFESLVTRSRRGLEEHYEPQLLRLLERREAGTRALQELQGEVQGMKEALRPLQGEVSRLRLQNRSLEEQIVLVKQKRDEEVGQYREQVEELEDRLKELKNSVQLQQRKNQELEELRSSLHHELSIYKGCLEIYGHLCKSEEKPDQEC

SEQ ID NO: 137 - protein, 482 aa, Sus scrofa
MASPEPRRGGDGAAQAARETRAEATSPLQEGNSESLYQLGTWNPEVTLSLEGTLNLEDILYLGDPVDLDEALYVEETEKPEETLHIEETRLPDEALYVEEPVKPEEMLYVEETVKPEEIVCVEQTMKPGETTSPEPITYGGETVPSEEKPNPEESLRAKPNPSTDGSLSLEDLELLEGRFQQCVQAVAQLEEERDQLIHELVLLREPALQEVQQVHQDILAAYKLHAQAELERDGLREEIRLVKQKLFKVTKECVAYQYQLECRRQDVAQFADFREVLTARAAQLSEELAQLRDAYQKQKEQLRQQLEAPPSQRDGHFLQESRRLSARFENLMAESRQGLEEEYEPQLLRLLERKEAAAKALQKTQAEIQEMKEALRPLQAEARQLHLQNRNLEDQITLVRQKRDEEVQQYREQLEEMEERQRQLKSGVQLQQQKNKEMEQLRISLAEELSTYKAMLPKSLERASAPTSEAGGIETQSQGAV

SEQ ID NO: 138 - protein, 388 aa, Bos taurus
MAAALIWISLLVGLLEGLGGTEAQQTTLHPLVGRVFVHTLDHESFLQRPEHVFSVSAPIPITYHAHLQGHPDLPRWLRYTQRSPYQPGFLYGTATPEDRGHQIIEVTAYNRDSFNTTQQMLVLLIGDPEGPLLPYQAEFLVRSHDVEEVLPSIPASRFLTALGGLWEPAELQLVNITSALDRGGRVPLPIEGRKEGVYIKVGSASPFSTCLKMVVSPDSHARCAQGQPPLLSCYDTLAPHFRVDWCNVSLVDKSVPEPADEAPAPGDGILEHDPFFCPPTEATNRDFLTDALVTLLVPLLVALLLTLLLAYVMCCRREGRLKRDLATSDIQMVHHRTIRGNTEELRQMASSREVPRPLSTLPMFNVHTGERLPPQVDSAQVPLILDQH

SEQ ID NO: 139 - protein, 291 aa, Bos taurus
MVREQYTTITEGTHIERPENQAVYKIGIYGWRKRCLYLFVLLLLIVLLVNFALTIWILRVMWFSPVGMGHLHVTADGLHLEGESEFLFPLYVKEIRSRVDSSLLLQSTQNVTMNARNTEGEVTGRLKVGPQMVEVQSQQFQIHSKDGKPLFTVDEEKVMVGTDKLRVTGPEGALFEHSVETPLVRPDPPQDLRLESPTRSLSMDAPKGIHIQAPAGKIEALTQMDIVLQSSDGTVVLDAETVCLPELALGSQGPAGSSQGLYEVCVCPDGKLYLSVAGMGTTCHEHSHLCL

SEQ ID NO: 140 - protein, 1135 aa, Gallus gallus
MPVFHTRTIESILEPVAQQISHLVIMHEEGEVDGKAIPDLTAPVSAVQAAVSNLVRVGKETVQTTEDQILKRDMPPAFIKVENACTKLVRAAQMLQADPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNTKSQGIEEALKNRNFTVEKMSAEINEIIRVLQLTSWDEDAWASKDTEAMKRALALIDSKMNQAKGWLRDPNAPPGDAGEQAIRQILDEAGKAGELCAGKERREILGTCKTLGQMTDQLADLRARGQGATPMAMQKAQQVSQGLDLLTAKVENAARKLEAMTNSKQAIAKKIDAAQNWLADPNGGSEGEEHIRGIMSEARKVAELCEEPKERDDILRSLGEISALTAKLSDLRRHGKGDSPEARALAKQIATSLQNLQSKTNRAVANTRPVKAAVHLEGKIEQAQRWIDNPTVDDRGVGQAAIRGLVAEGRRLANVMMGPYRQDLLAKCDRVDQLAAQLADLAARGEGESPQARAIAAQLQDSLKDLKARMQEAMTQEVSDVFSDTTTPIKLLAVAATAPSDTPNREEVFEERAANFENHAARLGATAEKAAAVGTANKTTVEGIQATVKSARELTPQVVSAARILLRNPGNQAAYEHFETMKNQWIDNVEKMTGLVDEAIDTKSLLDASEEAIKKDLDKCKVAMANMQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEHLHLTDELAPPKPPLPEGEVPPPRPPPPEEKDEEFPEQKAGEAINQPMMMAARQLHDEARKWSSKPVTVINEAAEAGVDIDEEDDADVEFSLPSDIEDDYEPELLLMPTNQPVNQPILAAAQSLHREATKWSSKGNDIIAAAKRMALLMAEMSRLVRGGSGNKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 141 - protein, 449 aa, Bos taurus
MEQKAGKSSEDGATVPPAAEAPEPAGSGGSAEEETAGPAESAARAGPATAGQQERAPAEDAVSAECQADGLGEVKAESQGEVELQQEDPGQGETAAAHGKTDRKDQTDSEPEGAEGDRETASASEEQSADEKEARLGSRETVDASREVQAEPKQASGAQEAEAGGGEAEGPQEEGGKTEEPQAEAREEAGAPAAQPDSEPGSPDEEQDQGAGAEAEDGAGGPPSSPEGWPESPTEEGGSASPEGLSPDTAASEELGPSASDSSPSDVPQSPTEPPPSEEKKKEKAPERRVSAPTRPRGPRAQNRKAIVDKFGGAAAGPTALFRNTKAAGAAVGGVRNMLLEWCRAMTRSYEHVDIQNFSSSWGSGMAFCALIHKFFPDAFDYAALDPAQRRHNFTLAFSTAEKLADCAQLLEVDDMVRLAVPDSKCVYTYIQELYRSLVQKGLVKTKKK

SEQ ID NO: 142 - protein, 315 aa, Gallus gallus
MPQNVILPGPAPWGFRLSGGIDFNQPLIITRITPGSKASTANLCPGDIIVAINGLSTENMTHNDAQERIKAAAHQLSLRIERAETKLWSPQVSEDGKANPYKINLEAEPQEFKPIGTAHNRRAQPFVAAANIDDKRQVVSSSYNSPIGLYSSGNIQDALHGQLRSLIPNASQNDPAPTAVPQSDVYRMLHSNQEEPSQPRQSGSFKVLQNLVSEEDGRPVGTRSVKAPVTKIPTGLPGAQKVPQCDKCGSGILGTVVKARDKYRHPECFVCSDCNLNLKQKGYFFVEGQLYCETHARARMRPPEGYEAVTVYPKC

SEQ ID NO: 143 - protein, 1744 aa, Gallus gallus
MDFGSVMNQAATPCSPAVNYELPSPGQSITKQVDTPDATRSPRGGQAHCKASRSMSVTAAMESSCELDLVYITERIIAVSYPSTAEEQSFRSNLREVAHMLKSKHGDNYVLFNLSERRHDISKLHPKVLDFGWPDLHTPALEKICSICKAMDTWLNAAAHNVVVLHNKGNRGRLGVVVAAYMHYSNISASADQALDRFAMKRFYEDKVVPVGQPSQKRYIHYFSGLLSGSIKMNNKPLFLHHVIMHGIPNFESKGGCRPFLKIYQAMQPVYTSGIYNVQGDSQTGICITIEPGLLLKGDILLKCYHKKFRSPTRDVIFRVQFHTCAVHDLDIVFGKEDLDEAFRDERFPEYGKVEFVFSYGPEKIQGMEHLENGPSVSVDYNTSDPLIRWDSYENFNIQREDSAEGTWAEPALPGKHLEKEVGHTQGPLDGSLYAKVKKKDSLHGSIGAVNTARLPLSAAPNHVEHTLSVSSDSGNSTASTKTDRTDEPGAPGAPTGHAVLSPEEKRDVDRLLVGFGLESAAPMHNHAPGPAPARLPAGPGRHVVPAQVHVNGAGTPLLAERETDILDDELPNQDGHSVGSLGTLSSLDGTTTASEAGFHEAPRVGSLSSLPNGPASYNGAEKMLKEGLYEAEPLSNGAYPYSNQNTLMGHHLRDPLAHLRPSRSAQEHLAGYPQRQPASASPAWLQPPVPQPYLYGYDLPSAHRSQSFPAVGTAKYEANLALPQAPARSTSSREAVQRGLNSWQQQGGSRPPSQLHDGGLESHSPSLSSCSPQPSPLQPMPPHSHSMPEFPRAPSRREIEQSIEALDVLMLDLAPSVHKSQSVPSAATRQDKPAAMLSSLSAQRLSGHYAQPTPQVVQPRSFGTSVGTDPLAKPYSPGPLVPAARSTAEPDYTVHEYRETYTPYSYQPVPEPRSYGSAPASILPLSASYSPAGSQQLLVSSPPSPTAPAQSQLPHKGLESYEDLSRSGEEPLNLEGLVAHRVAGVQSREKSPEESTVPARRRTPSDSHYEKSSPEPGSPRSPTVLSPEVVSTIAANPGGRPKEPHLHSYKEAFEEMESASPSSLTSGGVRSPPGLAKTPLSALGLKPHNPADILLHPVGELEGEAGADSEEEPRSYVESVARTATTGRAGNLPAAQPVGLEVPARNGAFGNSFTVPSPVSTSSPIHSVDGASLRSYPSEGSPHGTVTPPHAVAETAYRSPMVSQTPSAHSSYQTSSPSSFQAGTLGSPYASPDYPDGRGGFQPDPQARQQPQVSVVGVHALPGSPRTLHRTVATNTPPSPGFGRRAANPAVASVPGSPGLGRHTVSPHAPPGSPSLARHQMAAVPPGSPMYGYSSPEERRPTLSRQSSASGYQPPSTPSFPVSPAYYPGTSTPHSSSPDSAAYRQGSPTPQPALPEKRRMSAGERSNSLPNYATVNGKASSPLSSGMSSPSSGSAVAFSHTLPDFSKFSMPDISPETRANVKFVQDTSKYWYKPDISREQAIALLKDREPGAFIIRDSHSFRGAYGLAMKVASPPPTVMQQNKKGDITNELVRHFLIETSPRGVKLKGCPNEPNFGCLSALVYQHSIMPLALPCKLVIPDRDPMEEKKDAASTTNSATDLLKQGAACNVLFINSVEMESLTGPQAISKAVAETLVADPTPTATIVHFKVSAQGITLTDNQRKLFFRRHYPLNTVTFCDLDPQERKWTKTDGSGPAKLFGFVARKQGSTTDNVCHLFAELDPDQPAAAIVNFVSRVMLGSGQKR

SEQ ID NO: 144 - protein, 833 aa, Gallus gallus
MSEVGEEQNGGRGAGLGGITAALGLVLIPIPIPILVPVPIPIPIPIPPPQGRKGQSRHRALAGAGPGWGGVRAVTAPGAARCARPRAPQLRPARPERQQQPVSMVEHAEFSKAGKEPGLQIWRIEKFDLVPVPKNLYGDFFTGDSYLVLNTIRQRSGNLQYDLHFWLGDESSQDERGAAAIFTVQMDDYLQGKAVQHREVQGHESSTFLGYFKSGIKYKAGGVASGFRHVVPNEVTVQRLLQVKGRRTVRATEVPVSWESFNTGDCFILDLGSNIYQWCGSNSNRQERLKATVLAKGIRDNERNGRAKVFVSEEGAEREEMLQVLGPKPSLPQGASDDTKTDTANRKLAKLYKVSNGAGNMAVSLVADENPFSQAALNTEDCFILDHGTDGKIFVWKGRSANSDERKAALKTATDFIEKMGYPKHTQVQVLPESGETPLFKQFFKNWRDKDQTEGLGEAYISGHVAKIEKVPFDAATLHTSRAMAAQHGMEDDGSGKKQIWRIEGSEKVPVDPATYGQFYGGDSYIILYDYRHAGKQGQIIYTWQGAHSTQDEIATSAFLTVQLDEELGGSPVQKRVVQGKEPPHLMSMFGGKPLIVYKGGTSREGGQTTPAQTRLFQVRSSTSGATRAVELDPVASQLNSNDAFVLKTPSAAYLWVGRGSNSAELSGAQELLKVLGARPVQVSEGREPDNFWVALGGKAPYRTSPRLKDKKMDAHPPRLFACSNKSGRFTIEEVPGDLTQDDLATDDVMILDTWDQVFVWIGKDAQEEEKTEALKSAKRYIETDPASRDKRTPVTLVKQGLEPPTFSGWFLGWDDDYWSVDPLQRAMADVDV

SEQ ID NO: 145 - protein, 896 aa, Gallus gallus
MTVGCVPQPPFLGRTLLPVLLLAASARCHWPSEPAEVVRDWENQLEASMHSVLSDLRETVPAVVGIPDSSAVVGRFFRVSIPTDLIASNGEAVQVSEAGKESLPSWLHWNAESSSLEGLPLDTDKGVHYISVTTLQPFPNGSYVPQAANVFSVEVHQEDHSEPQSVRAAAQEAGDAAPFVCGAEEPVTILTVILDADLTKMTPKQRIELLNRMRSFSEVELHNMKLVPVVNNRLFDMSAFMAGPGNAKKVVENGALLSWKLGCSLSQNSVPNISKVEAPAKEGTMSARLGYPVVGWHIANKKPHLPKRMRRQINATPTPLTAIGPPTTAAQEPPTRIVPTPTSPAIAPPTETTAPPVREPIPLPRKPTVTIRTRGPIVQTPTLGPIQPTRLVEGTGTVSVPIRPTVPGYVEPTAVITPPTTTTKKPRVSTLKPATPSTTDSSTATTRRPTRRPKTPRPTKPPSTTRSTISKLTTASPPTRVRTTASGVPRPWEPNEPPKLTNHIDRVDAWEGTYFEVKIPSDTFYDKEDTTTDKLQLTLKLKEQQMIEENSWVQFNSTSQLMYGMPDRSHVGKHEYFMYATDKGGLFAVDAFEIHVHKRPHGDKSPVKFKARLEGDHSAVANDIHKKIMLVKKLALAFGDRNSSTITVQDIAKGSIVVEWTNNTLPLEPCPREQIRTLSKKIADDSGGPSPAFSNILQPEFKPLNVSVVGSGSCRHIQFVPVTKDGRVISEATPTLAAGKDPEKSSEDDVYLHTVIPAVVVAAILLVAGIIAMICYRKKRKGKLTIEDQATFIKKGVPIIFADELDDSKPPPSSSMPLILQEEKAPLPPPEYPNQSMPETTPLNQDTIGEYTPLRDEDPNAPPYQPPPPFTAPMEGKGSRPKNMTPYRSPPPYVPP

SEQ ID NO: 146 - protein, 1066 aa, Gallus gallus
MPVFHTRTIESILEPVAQQISHLVIMHEEGEVDGKAIPDLTAPVSAVQAAVSNLVRVGKETVQTTEDQILKRDMPPAFIKVENACTKLVRAAQMLQADPYSVPARDYLIDGSRGILSGTSDLLLTFDEAEVRKIIRVCKGILEYLTVAEVVETMEDLVTYTKNLGPGMTKMAKMIDERQQELTHQEHRVMLVNSMNTVKELLPVLISAMKIFVTTKNTKSQGIEEALKNRNFTVEKMSAEINEIIRVLQLTSWDEDAWASKDTEAMKRALALIDSKMNQAKGWLRDPNAPPGDAGEQAIRQILDEAGKAGELCAGKERREILGTCKTLGQMTDQLADLRARGQGATPMAMQKAQQVSQGLDLLTAKVENAARKLEAMTNSKQAIAKKIDAAQNWLADPNGGSEGEEHIRGIMSEARKVAELCEEPKERDDILRSLGEISALTAKLSDLRRHGKGDSPEARALAKQIATSLQNLQSKTNRAVANTRPVKAAVHLEGKIEQAQRWIDNPTVDDRGVGQAAIRGLVAEGRRLANVMMGPYRQDLLAKCDRVDQLAAQLADLAARGEGESPQARAIAAQLQDSLKDLKARMQEAMTQEVSDVFSDTTTPIKLLAVAATAPSDTPNREEVFEERAANFENHAARLGATAEKAAAVGTANKTTVEGIQATVKSARELTPQVVSAARILLRNPGNQAAYEHFETMKNQWIDNVEKMTGLVDEAIDTKSLLDASEEAIKKDLDKCKVAMANMQPQMLVAGATSIARRANRILLVAKREVENSEDPKFREAVKAASDELSKTISPMVMDAKAVAGNISDPGLQKSFLDSGYRILGAVAKVREAFQPQEPDFPPPPPDLEHLHLTDELAPPKPPLPEGEVPPPRPPPPEEKDEEFPEQKAGEAINQPMMMAARQLHDEARKWSSKGNDIIAAAKRMALLMAEMSRLVRGGSGNKRALIQCAKDIAKASDEVTRLAKEVAKQCTDKRIRTNLLQVCERIPTISTQLKILSTVKATMLGRTNISDEESEQATEMLVHNAQNLMQSVKETVREAEAASIKIRTDAGFTLRWVRKTPWYQ

SEQ ID NO: 147 - protein, 1822 aa, Gallus gallus
MKRKERIARRLEGIENDTQPMLLQNCPGSVTHRLLEEDTPRYMRATDPYSHHLGRSNEEEEASDSSVEKQPRSSRYRTETTGGNTESLYSTGNMDTHELESKAERIARYKAERRRQLAEKYGLSLDSDLDSDYSSRYSRARKDPDSVERKALRSERHDDENKDSGSLYLSRTEVKESKSVLSESREYSSREKDGIPAKEELSNEKSDKRADDDSAIRQASDPSATLDSSVSLTLSGRESSSCNEVPISPKQSPRESLSSPKRAASPIHLQNDQPLHSNIRQSESRFEMSTSGSALSAGGDRERGPRRPRRYFTPGENRKTSERFKTQPITSAERKETDRSIMCAEIPTADDEEKLDDRAKLSVAAKRLLFREMEKSFEMKNIPKPPTRSSAVERRMRRLQDRSHTQPITSEEVVIAATLQASAHQKILAKEEIRAAKEAMGQDQSDEPDSSTLSLAEKMALFNKLSQPVSRAISTRSRGDIRHRRMNTRYQTQPVTLGEVEQVQNESGKLTPLSSTVSTSVSAMASALSALFAGDMHAKSRSDGSVSAATKDELRFHASAESSDSSGKRTQKSEQWQPSMEVLESKRASKKHEEEGKRFLAHEANEIRKYSSFEDETTHPVLEKTGDYHKEAAYSFLRKGSMELFSSQPLFQPLERKEIDTIMQDHVEPTSELTSTPATRTLSQTTAAASCRLQELSEQLEGKFYKNSCEMFSAGENKIQTTEDATDSSSKTMSIKERLALLKKSGEEDWKSRLSKKQEYAKVSATDRSAQMQEVEQLLKKRIADNQESQMTIEERKHLITAREEAWKSRGKGAANDSTQFTVAGRMVKKGLASPSALTPVASPYGNRQKSTTPVTKPLEEIEARPDMQLESDMKLDKLESFLGRLNNKVAGMQETVLTVTGKSVKEVMKLDDDETFSKFYRRVDFPSSSVPLDLDEDFDAIFDPYAPKLISSVAEHKRAVRPMRRVQSSRNPLKMLAAREDILHEYTEQRLNVAFMESKRMKVEKMSANSNFSEVALAGLASKENFSNVSLRSVNLTEQNSNNSAVPYKKLMLLQIKGRRHVQTRLVEPRASSLNSGDCFLLLTPHLCFLWVGEFANVIEKAKASELATLIQTKRELGCRASYIQTIEEGINTHTHAAKDFWKLLGGQANYQSAGRPEEDEMYEAAIIETNCIYRLVEDKLIPEDDYWGKMPKCTLLQPKEVLVFDFGSEVYVWHGKEVTLAQRKVAFQLAKHLWNGTFDYSNCDINPLDPGECNPLIPRKGQGRPDWAVFGRLTEHNETILFKEKFLDWTELKKLNEKNSSESLHQKEESKSDSKPYDVMLMVPVPQTAVGTVLDGMNIGRGYGLVEGEDGRQFEIITASVDVWHILEFDYSRLPKQSIGQFHEGDTYVVKWKYMVSTTVGSRQKGEQQVRAVGKEKCVYFFWQGRHSTVSEKGTSALMTVELDEERGAQVQVLQGKELPCFLQCFQGGMIVHAGRREEEEENAQSDWRLYCVRGEVPNEGNLLEVACHCSSLRSRTSMIVLNINKALIYLWHGCKAQSHTKDVGRTAANKIKEQCPLEAGLHSSSKVTIHECDEGSEPLGFWDALGRRDRKAYDCMLQDPGKFNFTPRLFSLSSSSGEFSATEYVYPSRDPTVINSMPFLQEDLYTAPQPALFLVDNHHEVYLWQGWWPVENKITGSARIRWATDRKCAMETVLQYCKGKNVKKPPKSYLIHAGLEPLTFTNMFPSWEHREDIAEITEMDADVSNQIILVEDVLAKLCKTVYPLADLLARPLPEGVDPLKLEIYLSDEDFEVALEMTREEYNALPSWKQVNLKKAKGLF

SEQ ID NO: 148 - protein, 140 aa, Gallus gallus
MAGWQSYVDNLMCDGCCQEAAIVGYCDAKYVWAATAGGIFQSITPVEIDMIVGKDREGFFTNGLTLGAKKCSVIRDSLYVDGDCTMDIRTKSQGGEPTYNVAVGRAGRVLVFVMGKEGVHGGGLNKKAYSMAKYLRDSGF

SEQ ID NO: 149 - protein, 361 aa, Bos taurus
MEHNGSASNADKIHQNRLSNVTEDEDQDAALTIVTVLDKVAAIVDSVQASQKRIEERHRVMENAIKSVQIDLLKFSQSHSNTGYVINKLFEKTRKVSAHIKDVKARVEKQQTHVKKVEAKQEEIMKKNKFRVVIFQEEVQCPTSLSVVKDRSLTESPEEVDEIFDTPVDLSSDEEYFVEESRSARLKKSGKERIDNIKKAFSKENMQKTRQNFDKKVNRIRTRIVTPERRERLRQSGERLRQSGERLKQSGERFKKSISNAAPSREAFKMRSLRKTKDRAVAEGPEEVREMGVDIIARGEALGPISELYPEALSETDPEEASATHPPQEGGEVSTPEPLKVTFKPQVKVEDDESLLLDLKQ

SEQ ID NO: 150 - protein, 1667 aa, Mus musculus
MSLPFYQRSHQHYDLSYRNKDLRTTMSHYQQEKKRSAVYTHGSTAYSSRSLAARRQESEAFSQASATSYQQQASQTYSLGASSSSRHSQGSEVSRKTASAYDYGYSHGLTDSSLLLEDYSSKLSPQTKRAKRSLLSGEETGSLPGNYLVPIYSGRQVHISGIRDSEEERIKEAAAYIAQKTLLASEEAIAASKQSTASKQSATSKRTTSTLQREETFEKKSRNIAIREKAEELSLKKTLEETQTYHGKLNEDHLLHAPEFIIKPRSHTVWEKENVKLHCSVAGWPEPRLTWYKNQVPINVHANPGKYIIESRYGMHTLEISKCDFEDTAQYRASAMNVQGELSAYASVVVKRYKGELDESLLRGGVSMPLSFAVTPYGYASKFEIHFDDKFDVSFGREGETMSLGCRVVITPEIKHFQPEVQWYRNGAPVSPSKWVQPHWSGDRATLTFSHLNKEDEGLYTIRVRMGEYYEQYSAYVFVRDADAEIEGAPAAPLDVVSLDANKDYIIISWKQPAVDGGSPILGYFIDKCEVGTDTWSQCNDTPVKFARFPVTGLIEGRSYIFRVRAVNKTGIGLPSRVSEPVAALDPAEKARLKSHPSAPWTGQIIVTEEEPTEGVIPGPPTDLSVTEATRSYVVLSWKPPGQRGHEGIMYFVEKCDVGAENWQRVNTELPVKSPRFALFDLVEGKSYRFRVRCSNSAGVGEPSETTEVTVVGDKLDIPKAPGKIIPSRNTDTSVVVSWEESRDAKELVGYYIEASVVGSGKWEPCNNNPVKGSRFTCHGLTTAQSYIFRVRAVNAAGLSEYSQDSEAIEVKAAIGGGVSPDVWPQLSDTPGGLTDSRGGMNGASPPTSQKDALLGSNPNKPSPPSSPSSRGQKEVSTVSESVQEPLSSPPQEAAPEEEQSQSEPPKKKKDPVAVPSAPYDITCLESFRDSMVLGWKQPDTTGGAEITGYYVNYREVVGEVPGKWREANIKAVSDAAYKISNLKENTLYQFQVSAMNIAGLGAPSTVSECFKCEEWTIAVPGPPHSVKLSEVRKNSLVLQWKPPVYSGRTPVTGYFVDLKEASAKDDQWRGLNEAAIVNKYLRVQGLKEGTSYVFRVRAVNQAGVGKPSDLAGPVVAETRPGTKEVVVSVDDDGVISLNFECDQMTPKSEFVWSKDYVPTEDSPRLEVENKGDKTKMTFKDLGTDDLGTYSCDVTDTDGIASSYLIDEEEMKRLLALSQEHKFPTVPTKSELAVEILEKGQVRFWMQAEKLSSNAKVSYIFNEKEIFEGPKYKMHIDRNTGIIEMFMEKLQDEDEGTYTFQIQDGKATGHSTLVLIGDVYKKLQKEAEFQRQEWIRKQGPHFAEYLSWEVTGECNVLLKCKVANIKKETHIVWYKDEREISVDEKHDFKDGICTLLITEFSKKDAGFYEVILKDDRGKDKSRLKLVDEAFQDLMTEVCKKIALSATDLKIQSTAEGIRLYSFVCYYLDDLKVNWSHNGTGIKYTDRVKSGVTGEQIWLQINEPTPNDKGKYVMELFDGKTGHQKTVDLSGQAFDEAFAEFQRLKQAAIAEKNRARVLGGLPDVVTIQEGKALNLTCNVWGDPPPEVSWLKNEKPLTSDDHCSLKFEAGKTAFFTISGVSTADSGKYGLVVKNKYGSETSDFTVSVFIPEEELRKGAMEPPKGNQKSK

SEQ ID NO: 151 - protein, 2335 aa, Bos taurus
QPADMTSATEFENVGNQPPYSRINARWDGPDDELDNDNSSARLFERSRIKALADEREVVQKKTFTKWANSHLVHVSCRITDLYKDLRDGRMLIKLLEVLSGEMLPKPTKGKMRIHCLENVDKALQFLREQRVHLENMGSHDIVDGNHRLVLGLIWTIILRFQIQDIVVQTQEGRETRSAKDALLLWCQMKTAGYPHVNVTNFTSSWKDGLAFNALIHKHRPDLIDFDKLKDSNARHNLEHAFEVAERELGIIPLLDPEDVFTENPDEKSIITYVVAFYHYFSKMKVLAVEGKRVGKVIDHAIETEKMIEKYSGLASDLLTWIEQTITILNSRKFANSLAGVQQQLQAFSTYRTVEKPPKFQEKGNLEVLLFTIQSRMRANNQKVYTPHDGKLVSDINRAWESLEEAEYRRELALRDELIRQEKLEQLARRFDRKAAMRETWLNENQRLVAQDNFGYDLAAVEAAKKKHEAIETDTAAYEERVRALEDLAQELERENYHDQKRIMARKDNILRLWDYLQELLQARRQRLEKTLDLQKLFQDMLHSIDWMDGIKAHLLSAEFGKHLLEVEDLLQKHKLMEADIAIQGDKVKAITTATLQFTEGPGYQPCDPQVIQDRVSHLEQCFEELSNTAAGRKAQLEQSKQLCKFFWEMDEAESWIKEKEQIYSSLDYGKDLTSVLILQRKHKAFEDELRGLDAHLDQIFREAEGMVARKQFGHERIEVRIKKVSDQWNELKDLAAFCKKNLQDTENFFQFQGDADDLKAWLQDAHRLLSGEDVGHDEGATRALGKKHKDFLEELEESRGVMEHLEQQAQAFPQGFQDSPDVTNRLQALRDLYQQVVAQADMRQQRLQDALDLYTVFGETDACELWMGEKGKWLAQMEIPDTLEDLEVVQHRFDILDQEMKTLMTQIDGVNLAANSLVESNHPRSAEVKKYQDRLNTRWQDFQTMVSERREAVDSALRVHNYCVDCEETGKWIADKTKVVESTKDLGRDLAGVIAIQRKLSGLERDVAAIQARLGTLERESQQLMASHPELKEDIEQRQAYVEELWQGLMQALKSQEASLGEASQLQAFLQDLDAFQAWLSTTQKDVASKDMPESLPEAEQLLQQHAAIKDDIDRHQESYQKVRVSGEKVTHGQTDPEYLLLGQRLDGLEKGWDALSRMWESRSQALTQCLGFQEFQKDAKQAEAILSNQEYTLAHLEPPDSLEAAEAGIRKFEDFLLSMENNRDKVLSPVDSGNKLVAEGNLYSDKIKEKVQLIEDRHRKNNEKAQEASDLLKDNLELQNFLQNCQELTLWINDKLLTSQDVSYDEARNLHNKWLKHQAFVAELASHQGWLENIDAEGKQLMEEKPQFAVLVSQKLEALHQLWDELQANTQEMAQHLSAARSSDLRSQTQADLNKWIRAMEDQLQSDDLGKDLTSVNRMLAKLKRVEDQVNVRKEELQELFAQMPSLGEEAGDEALSIEKRFLDLLEPLGRRKKQLESSRAKLQISRDLEDETLWVEERLPLAQSTDYGTNLQTVQLFMKKNQTLQNEILGHTPRVEDVLHRGQQLVAAAEIDCQDVEERLGKLQGSWDTLQEAAASRLQHLREASEAQQYYLDAGEAEAWIGEQEFYVFSDENPQDEESAIVMLKRHLRQQRAVDEYGRNIKQLAGRAQGLLSAGHPEGEQIIRLQGQVDKQYAGLKDMAEERRRKLENMYHLFQLKREVDDLEQWITEKDTVASSSEMGQDFDHVTVLRDKFRDFARETGAIGQERVDNVNAIIERLIDAGHSEAATIAEWKDGLNEMWADLLELIDTRMQLLAASYDLQRYFYTSSEILGLIGEKHRELPEDVGLDASTAESFHRTHTAFERELHLLGEQVQQFQDVAARLQTAYAGEKADIIQNKEQEVSAAWQALLDACAGRRTQLVDTADKFRFFSMARDLLSWMESIVRQIETQEKPRDVSSVELLMKYHQGIQAEIETRSKNFSTCLELGESLLQRQHQASEEIREKREQVVSRREEMQEKWEARWERLRMLLEVCQFFRDASVAEAWLIAQEPYLASRDYGHTVDSVEKLIKRHEAFEKSAASWEGRFAALEKPTTLELKERQTPERTEEEPGLQEEEGETAGEAPRGPHQATTERTSPGEEERPWPQDLQPPPPPGPHEDGQEEKSSTDERPATEPLFKVLDTPLSEGDEPTTLPAQRDRGHSVQMEGFLGRKHDLEGPNKKASNRSWNNLYCVLRNSELAFYKDAKNLALGVPYHGEEPLALRHAICEIAANYKKKKHVFKLRLSNGSEWLFHGKDEEEMLSWLQGVSTAINESQSIRVKAQSLPLPPITGPEASLGKKDKEKRFSFFPKKK

SEQ ID NO: 152 - protein, 1547 aa, Bos taurus
MLSWRLHTGPEKAELQELNARLYDYVCRVRELERENLLLEEELRSRRGQEGLWAEAEARCTEEARGLRRQLDELSWATALAQGERDALRRELRELQLLGEETRAARGRLDAELDSQRRELQEELAARAALEALLGRLQAERRGLDAARERDVRELRARAAGLTLRYRGRVAGPAAPPLRLRELHEDCALLVTEAWRETVQRYEDEVRELEEALRRGRESRREAEEETRLCAQEAEALRREVLELEQLRALLEEELLRVREASELQAEERQREIAYLEDEKAALTLAMADRLRDYQDLVQVKTGLSLEVATYRALLEGESNPEILIVECIENMPQEFREKSYQYTTSVLQKENERNLFPRQKAPPASFRQSLAPRSQTPVRSDARRDVLGSGYSSFTTAWQENAYQKTASGQTNFRTFSPTPGLLRNTEAQLKTFPERPRTEGTKAPPASSAKESAAAAEPYQERRAEEAAAASEGTWSNERTVIWGKKTEAKATKEQERNRPGTIQTKREEKMFDSKEKASEERNLRWEELTKLDKEARKRESEQMREKAREKESLKEESVKGREIPIRLEASQDSTAKVAPQDLQTPLKEDAGDGTGRGVETREAGFTLGASDTTASLKGGSMTETIAENIVSSILKQFTHSPEAEASAESFPDTKVTYVDRKEVPGDRKAKTEIVVESKLTEEIDISDEAGLDYLLSKDAKEVELKGKSAERLIGDVIRLGLKGKEGRAKVVNVEIIEEPMSYVSSEKEDEFSTPFEVEEIDDVSPSSRGLVEEREEAVYGESDVTCSGDAGQEARRPQESVTHVEEVTEAGDLEGEQSYFVSTPDEHPGAPEREEGSVYGQIHIEEESTIRYSWQDEIVPGSRRRVRRDDVPGEKVVKPVDVPEVPLEGDTASAPWKEQTRSGEFHAKPIVIEKEIKIPHEFHTSIKEGSKEPRHQLVEVIEQLEENLPERMKEELSALTREGQGGPAGVSFDVKKVQSAGSGAVTLVAEVNLSQTVDADQLDLEELSKDEAGEIEKAVESVVRDSLARRRSPGPGSPEAEAGGGFRRWATQELYSSSAGEADAGLASPPGPVSATVEVTSPTGFVHAHVLEDVSQSGGRVQIASPGMWRTEQVSAEGRAAQAVEVSAEGQASWAEGSAGTSRSARLITLGARQSPVSTEVIFPGPDAACPEAGGTEEPGPAELPTEPPRFGRHSPFGSQQFYAQREVIFQAPVSGVGKAGDSSQAEESAGTQTSVKHLQLGTREGLTEQTQLTAPLSGAVELGVREASVLMEAWSGDGTSIRHVTIGPQRHQATEPIVPPPLEFSDSESSTHREGSADETLATSSYTVGRNILVTEKSTFQRAVSESPQEASAEDMSGNEVTSGVSRSFRHIQLGPAEAKTSEHIVFHGPISKTFALAGSVDSPEVSEVADSGRTLRHVALGPKETSFTFQMDVSNVEATSRWTQEARVLFPAGTEAEAPSVSEPGAWRDASSRNDLAAGVSFQGSAGDRHQAPGERGKEQAEFDKTVQLQRMVDQRSVISDEKKVALLYLDSQEEENEGHWF

SEQ ID NO: 153 - protein, 4644 aa, Bos taurus
  MOD_RES 1109: note = Any amino acid
MVAGMFMPLDQLRAIYEVLFREGVMVAKKDRRPRSLHPHVPGVTNLQVMRAMASLRARGLVRETFAWRHFYWYLTNEGIAHLRQYLHLPPEIVPASLQRVRRPVAMVMPARRTPHVQAVQGPLGCPPKRGPPPTEDPAREERCVYRRKEPEEGAPEPPVVPAATPGTLARQGLEPAPPTDERDRVQKKTFTKWVNKHLIKAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTSSWRDGRLFNAIIHRHKPMLIDMNKVYRQTNLENLDQAFSVAERDLGVTRLLDPEDVDVPQPDEKSIITYVSSLYDAMPRVPDVQDGVKANELQLRWQEYRELVLLLLQWIRAHTAAFEERRFPSSFEEIEILWCQFLKFKETELPAKEADKNRSKGIYQSLEGAVQAGQLKVPPGYHPLDVEKEWGKLHVAILEREKQLRSEFERLERLQRIVSKLQMEAGLCEEQLNQADALLQSQDVRLLAAGKAPQRAGEVERDLDKADGMIRLLFNDVQALKDGRHPQGEQMYRRVYRLHERLVAIRTEYNLRLRGTPRHPELEDSTLRYLQDLLAWVEENQRRLDGAEWGVDLPSVEAQLGSHRGLHQSVEEFRAKIERARTDEGQLSPATRGAYRDCLGRLDLQYAKLLNSSKARLRSLESLHGFVAAATKELMWLSEKEEEEVGFDWSERNSNMAAKKEAYSALMRELELKEKKIKEIQSTGDRLLREDHPARPTVESFQAALQTQWSWMLQLCCCIEAHLKENTAYFQFFSDVREAEEQLRKLQETLHRKYTCDRSITVTRLEDLLQDAQDEKDQLNEYRGHLSGLAKRAKAIVQLTPRNPTQPTRGRVPLLAVCDYKQVEATVHKGDECQMLGPAQPFHWKVLSGSGSEAAVPSVCFLVPPPNQEALEAVARLEAQHQALVTLWHQLHTDMKSLLAWQSLSRDVQLIRSWSLVTFRTLKPEEQRQALRSLELHYQAFLRDSQDAGGFGPEDRLQAEREYGSCSRHYQQLLQSLEQGKCGQCGXLDKEPARECAQRIAEQQKAQAEVEGLGKGVARLSAEAEKVLALPEPSPAAPTLRSELELTLGKLEQVRSLSAIYLEKLKTISLVIRSTQGAEEALKAHEEQLKEAQAVPAALPELEATKAAMKKLRAQAEAQQPVFDALRDELRGAQEVGERLQQRHGERDVEVERWRERVTQLLERWQAVLAQTDVRQRELEQLGRQLRYYRESADPLGAWLQDARRRQEQIQAVPLADSQAVREQLRQEKALLEEIERHAEKVEECQRFAKQYINAIKDYELQLVTYKAQLEPVASPAKKPKVQSGSESVIQEYVDLRTRYSELSTLTSQYIRFISETLRRMEEEERLAEQQRAEERERLAEVEAALEKQRQLAEAHAQAKAQAEREAQELQRRMQEEVARREEVVVDAQQQKRSIQEELQQLRQSSEAEIQAKARQVEAAERSRLRIEEEIRVVRLQLETTERQRGGAEGELQALRARAEEAEAQKRQAQEEAERLRRQVQDETQRKRQAEAELGLRVKAEAEAAREKQRALQALEELRLQAEEAERRLRQAEAERARQVQVALETAQRSAQAELQSKHASFAEKTAQLERTLEEEHVTVVQLREEATRREQQQAEAERAREEAERELERWQLKANEALRLRLQAEEVAQQKSLAQAEAEKQKEAAEREARRRGKAEEQAVRQRELAEQELERQRQLAEGTAQQRLAAEQELIRLRAETEQGEQQRQLLEEELARLQSEAAAATQKRQELEAELAKVRAEMEVLLASKARAEEESRSSSEKSKQRLEAEAGRFRELAEEAARLRALAEEAKRQRQLAEEDAARQRAEAERVLSEKLAAISEATRLKTEAEIALKEKEAENERLRRLAEDEAFQRRRLEEQAAQHKADIEERLAQLRKASESELERQKGLVEDTLRQRRQVEEEILALKASFEKAAAGKAELELELGRIRGNAEDTLRSKEQAEQEAARQRQLAAEEERRRREAEERVQKSLAAEEEAARQRKAALEEVERLKAKVEEARRLRERAEQESARQLQLAQEAAQKRLQAEEKAHAFAVQQKEQELQQTLQQEQSMLERLRGEAEAARRAAEEAEEARERAEGEAAQSRQRVEEAERLKQAAEEQAQAQAQAQAAAEKLRKEAEQEAARRAQAEQAALRQKQAADAEMEKHKKFAEQTLRQKAQVEQELTALRLKLEETDHQKSILDQELQRLKAEVTEAARQRSQVEEELFSVRVQMEELGKLKARIEAENRALILRDKDNTQRLLQEEAEKMKQVAEEAARLSVAAQEAARLRQLAEEDLAQQRALAEKMLKEKMQAVQEATRLKAEAELLQQQKELAQEQARRLQEDKEQMAQQLAQETQGFQRTLETERQRQLEMSAEAERLRLRVAEMSRAQARAEEDAQRFRKQAEEISAKLHRTELATQEKVTLVQTLETQRQQSDRDADRLREAIAELEREKDKLKKEAELLQLKSEEMQTVQQEQLLQETQALQQSFLSEKDSLLQRERFIEEEKAKLERLFQDEVAKAQKLREEQQRQQQQMQQEKQQLLASMEEARRRQREAEEGVRRKQEELQLLEQQRQQQEQLLAEENRRLRERLEHLEEEHRAALAHSEEITAAQAAATRALPNGQDATDGPAAEPEHAFEGLRQKVPAQQLQEAGILSTEEVQRLVQGHTTVAELTQREDVRRYLQGHSSIAGLLLKPANEKLTIYAALRRQLLSPGTAVILLEAQAASGFLLDPVRNRRLTVNEAVKEGVVGPELHHKLLSAERAVTGYKDPYTGEQISLFQAMKKDLIVREHGIRLLEAQIATGGVIDPVHSHRVPVDVAYQRGYFDEEMNRVLQDPSDDTKGFFDPNTHENLTYLQLLERCVEDPETGLRLLPLTDQAAKGGELVYTDSEARDVFEKATVSAPFGKFQGKTVTIWELINSEYFTAEQRRDLLRQFRTGKVTVEKIIKIVITVIEEHEQKGQLCFQGLRALVPAAELLESGVIDWDLFRQLQLGERSVQEVAEVEAVRRALRGSGVIAGVWLEEARQKLSIYEALKKELLQPEAAVALLEAQAGTGHVIDPATSARLTVDEAVRAGLVGPELHEKLLSAEKAVTGYKDPYSGQSVSLFQALKKGLIPREQGLRLLDAQLSTGGIVDPSKSHRLPLDVACARGYLDKETSTALTAPRDDAKTYYNPRTWEPATYSQLQQQCRPDPLTGLSLLPLSEEAARARQQELYSEVQAREAFQKATVEVPVGSFQGRAVTIWELINSEYFTAEQRQELLRQFRTGKVTVEKIIKIIITIVEEVETTRRERLSFSGLRAPVPASELLASGILSSSQFEQLKDGKTSVKDLSELDSVRTLLQGSGCLAGIYLEESKEKVTIYEAMRRGLLRPSTAILLLEAQAATGFLVDPVRNQRLYVHEAVKAGVVGPELHEKLLSAEKAVTGYKDPYSGSTISLFQAMKKGLVVREHGIRLLEAQIATGGIIDPVHSHRVPVDVAYQRGYFDKEMNRVLEDPSDDTKGFFDPNTRENLTYRQLLERCVEDPETGLRLLPLKGPEKAEVVETTRVYTEEETRRAFEETQIDIPGGGSHGGSTMSLWEVMQSDLIPEEQRAQLMADFQAGRVTKERMIIIIIEIIEKTEIVRQQNLASYDYVRRRLTAEDLHEARVISRESYSLLREGTRSLREVLEAESAWRYLYGTGCVAGVYLPGSRQTLTIYQALKKGLLSAEVARLLLEAQAATGFLLDPVKGDRLTVDEAVRKGLVGPELHDRLLSAERAVTGYRDPYTEQTISLFQAMKKDLIPAEEALRLLDAQLATGGIVDPRLGFHLPLEVAYQRGYLNKDTHDQLSEPSEVRSYIDPSTDERLSYTQLLRRCRRDEASGLFLLPLSDARKLTFRGLRKQITVEELVRSHVMDEATAQRLQEGLTSIEEVSKNLQKFLEGTSSIAGVLVDATKERLSVYQAMKKGIIRPGTAFELLEAQAATGYVIDPIKGLKLTVEEAVRMGIVGPEFKDKLLSAERAVTGYKDPYSGKLISLFQAMKKGLILKDHGIRLLEAQIATGGIIDPEESHRLPVDVAYQRGLFDEEMNEILTDPSDDTKGFFDPNTEENLTYLQLMERCVTDPQTGLRLLPLKEKKRERKTSSKSSVRKRRVVIVDPETGKEMSVYEAYRKGLIDHQTYLELSEQECEWEEITISSSDGVVKSMIIDRRSGRQYDIDEAIAKSLIDRSALDQYRAGTLSITEFADMLSGNAGGFRSRSSSVGSSSSYPISPAVSRTQLASWSDPTEETGPVAGILDTETLEKVSITEAMHRNLVDNITGQRLLEAQACTGGIIDPNTGERFPVTEAVNKGLVDKIMVDRINLAQKAFCGFEDPRTKTKMSAAQALKKGWLYYEAGQRFLEVQYLTGGLIEPDTPGRVPLDEALQRGTVDARTAQKLRDVSAYSKYLTCPKTKLKISYKDALDRSMVEEGTGLRLLEAATQSSKGYYSPYSVSGSGSTTGSRSGSRTGSRAGSRRGSFDATGSGFSMTFSSSSYSSSGYGRRYASGPTSSLGGPESTAA

SEQ ID NO: 154 - protein, 1116 aa, Rattus norvegicus
MARIPRCDFLGLPGICYLLSFLLAGLLLPRASAFNLDVMGAIRKEGEPGSLFGFSVALHRQLQPRPQSWLLVGAPQALALPGQQANRTGGLFACPLSLEETDCYRVDIDRGANVQKESKENQWLGVSVRSQGAGGKVVTCAHRYESRQRVDQVLETRDVIGRCFVLSQDLAIRDELDGGEWKFCEGRPQGHEQFGFCQQGTAATFSPDSHYLIFGAPGTYNWKGLLFVTNIDSSDPDQLVYKTLDPADRLTGPAGDLTLNSYLGFSIDSGKGLMRSEELSFVAGAPRANHKGAVVILRKDSASRLIPEVVLSGERLTSGFGYSLAVTDLNSDGWADLIVGAPYFFERQEELGGAVYVYMNQGGHWADISPLRLCGSPDSMFGISLAVLGDLNQDGFPDIAVGAPFDGDGKVFIYHGSSLGVVTKPSQVLEGEAVGIKSFGYSLSGGLDVDGNHYPDLLVGSLADTAALFRARPVLHVSQEIFIDPRAIDLEQPNCADGRLVCVHVKVCFSYVAVPSSYSPIVVLDYVLDGDTDRRLRGQAPRVTFPGRGPDDLKHQSSGTVSLKHQHDRVCGDTVFQLQENVKDKLRAIVVTLSYGLQTPRLRRQAPDQGLPLVAPILNAHQPSTQRTEIHFLKQGCGDDKICQSNLQLAQAQFCSRISDTEFQALPMDLDGTALFALSGQPFIGLELTVTNLPSDPARPQADGDDAHEAQLLATLPASLRYSGVRTLDSVEKPLCLSNENASHVECELGNPMKRGTQVTFYLILSTSGITIETTELKVELLLATISEQDLHPVSVRAHVFIELPLSISGVATPQQLFFSGKVKGESAMRSERDVGSKVKYEVTVSNQGQSLNTLGSAFLNIMWPHEIANGKWLLYPMRVELEGGQGPEKKGICSPRPNILHLDVDSRDRRRRELGQPEPQEPPEKVEPSTSWWPVSSAEKRNVTLDCAQGTAKCVVFSCPLYSFDRAAVLHVWGRLWNSTFLEEYMSVKSLEVIVRANITVKSSIKNLLLRDASTVIPVMVYLDPVAVVAEGVPWWVILLAVLAGLLVLALLVLLLWKCGFFRRNSPSSSFPANYHRAHLAVQPSAMEAGGPGTVGWDSSSGRSTLRPLYPSTTQ

SEQ ID NO: 155 - protein, 801 aa, Bos taurus
MNLQLIFWIGLISSVCCVFGQADENRCLKANAKSCGECIQAGPNCGWCTNSTFLQEGMPTSARCDDLEALKKKGCHPNDIENPRGSKDIKKNKNVTNRSKGTAEKLQPEDITQIQPQQLVLQLRSGEPQTFTLKFKRAEDYPIDLYYLMDLSYSMKDDLENVKSLGTDLMNEMRRITSDFRIGFGSFVEKTVMPYISTTPAKLRNPCTNEQNCTSPFSYKNVLSLTDKGEVFNELVGKQRISGNLDSPEGGFDAIMQVAVCGSLIGWRNVTRLLVFSTDAGFHFAGDGKLGGIVLPNDGQCHLENDVYTMSHYYDYPSIAHLVQKLSENNIQTIFAVTEEFQPVYKELKNLIPKSAVGTLSANSSNVIQLIIDAYNSLSSEVILENSKLPEGVTINYKSYCKNGVNGTGENGRKCSNISIGDEVQFEISITANKCPNKNSETIKIKPLGFTEEVEIILQFICECECQGEGIPGSPKCHDGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTTSCMAVNGQICNGRGVCECGACKCTDPKFQGPTCEMCQTCLGVCAEHKECVQCRAFNKGEKKDTCAQECSHFNITKVENRDKLPQPGQVDPLSHCKEKDVDDCWFYFTYSVNGNNEATVHVVETPECPTGPDIIPIVAGVVAGIVLIGLALLLIWKLLMIIHDRREFAKFEKEKMNAKWDTQENPIYKSPINNFKNPNYGRKAGL

SEQ ID NO: 156 - protein, 803 aa, Gallus gallus
MAETNLTLLTWAGILCCLIWSGSAQQGGSDCIKANAKSCGECIQAGPNCGWCKKTDFLQEGEPTSARCDDLAALKSKGCPEQDIENPRGSKRVLEDREVTNRKIGAAEKLKPEAITQIQPQKLVLQLRVGEPQTFSLKFKRAEDYPIDLYYLMDLSYSMKDDLENVKSLGTALMREMEKITSDFRIGFGSFVEKTVMPYISTTPAKLRNPCTGDQNCTSPFSYKNVLSLTSEGNKFNELVGKQHISGNLDSPEGGFDAIMQVAVCGDQIGWRNVTRLLVFSTDAGFHFAGDGKLGGIVLPNDGKCHLENNMYTMSHYYDYPSIAHLVQKLSENNIQTIFAVTEEFQAVYKELKNLIPKSAVGTLSSNSSNVIQLIIDAYNSLSSEVILENSKLPKEVTISYKSYCKNGVNDTQEDGRKCSNISIGDEVRFEINVTANECPKKGQNETIKIKPLGFTEEVEIHLQFICDCLCQSEGEPNSPACHDGNGTFECGACRCNEGRIGRLCECSTDEVNSEDMDAYCRRENSTEICSNNGECICGQCVCKKRENTNEVYSGKYCECDNFNCDRSNGLICGGNGICKCRVCECFPNFTGSACDCSLDTTPCMAGNGQICNGRGTCECGTCNCTDPKFQGPTCEMCQTCLGVCAEHKDCVQCRAFEKGEKKETCSQECMHFNMTRVESRGKLPQPVHPDPLSHCKEKDVGDCWFYFTYSVNSNGEASVHVVETPECPSGPDIIPIVAGVVAGIVLIGLALLLIWKLLMIIHDRREFAKFEKEKMNAKWDTGENPIYKSAVTTVVNPKYEGK

SEQ ID NO: 157 - protein, 798 aa, Sus scrofa
MNLQLIFWIGLISSVCYVFGQADENRCLKANAKSCGECIQAGPNCGWCTNSTFLQEGMPTSARCDDLEALRKKGCHPDDIENPRGSKNIKKNKNVTNRSKGTAEKLQPEDITQIQPQQLVLQLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTDKGEVFNELVGKQRISGNLDSPEGGFDAIMQVAVCGSLIGWRNVTRLLVFSTDAGFHFAGDGKLGGIVLPNDGHCHLENDVYTMSHYYDYPSIAHLVQKLSENNIQTIFAVTEEFQPVYKELKNLIPKSAVGTLSANSSNVIQLIIDAYNSLSSEVILENSKLPEGVTINYKSYCKNGVNGTGENGRKCSNISIGDEVQFEISITANKCPNKNSETIKIKPLGFTEEVEIILQFICECECQSEGIPSSPKCHDGNGTFECGACRCNEGRVGRHCECSTDEVNSEDMDAYCRKENSSEICTNNGECVCGQCVCRKRDNTNEIYSGKFCECDNFNCDRSNGLICGGNGVCKCRVCECNPNYTGSACDCSLDTTSCMAVNGQICNGRGVCECGVCKCTDPKFQGPTCEMCQTCLGVCAEHKECVQCRAFNKGEKKDTCAQECSHFNITKVENRDKLPQPGQVDPLSHCKEKDVDDCWFYFTYSVNGNNEATVHVVETPECPTGPDIIPIVAGVVAGIVLIGLALLLIWKLLMIIHDRREFAKFEKEKMNAKWDTGENPIYKSAVTTVVNPKYEGK

SEQ ID NO: 158 - protein, 357 aa, Bos taurus
WQKHLAGRGWGPWHIKPPGPAEAGCDGTMADSEEVQRATALIEERLAQEEENEKLRGTTHQKLPMEMLVLEDEKHHRPESPSLQKVKGQERVRKTSLDLRREIIDVGGIQNLIQLRKKRKQKKREALAASQEPPPEPEEITGPVDEETFLKAAVEGKMKVIEKFLADGGSPDTCDQFRRTALHRASLEGHMEILEKLLESGATVDFQDRLDCTAMHWACRGGHLEVVRLLQSRGADTNVRDKLLSTPLHVAVRTGQVEIVEHFLSLGLDINAKDREGDSALHDAVRLNRYKIIKLLLLHGADMMSKNLAGKTPTDLVQLWQADTRHALENPEPGSEQNGLEGSTESGRETPQPVAAE

SEQ ID NO: 159 - protein, 2043 aa, Gallus gallus
MAAPAPPAPGGGTSPPAGPPRLRQSDSNASFLRAARAGNLDKVVEYLKSGIDINTCNQNGLNALHLAAKEGHVGLVQELLERGSAVDSATKKGNTALHIASLAGQAEVVKVLVKEGANINAQSQNGFTPLYMAAQENHIEVVKYLLENGANQSTATEDGFTPLAVALQQGHNQAVAILLENDTKGKVRLPALHIAARKDDTKSAALLLQNDHNADVQSKMMVNRTTESGFTPLHIAAHYGNVNVATLLLNRGAAVDFTARNGITPLHVASKRGNTNMVKLLLDRGGQIDAKTRDGLTPLHCAARSGHDQVVELLLERGAPLLARTKNGLSPLHMAAQGDHVECVKHLLQHKAPVDDVTLDYLTALHVAAHCGHYRVTKLLLDKRANPNARALNGFTPLHIACKKNRIKVMELLVKYGASIQAITESGLTPIHVAAFMGHLNIVLLLLQNGASPDVTNIRGETALHMAARAGQVEVVRCLLRNGALVDARAREEQTPLHIASRLGKTEIVQLLLQHMAHPDAATTNGYTPLHISAREGQVDVASVLLEAGASHSMSTKKGFTPLHVAAKYGSLEVAKLLLQRRASPDSAGKNGLTPLHVAAHYDNQKVALLLLEKGASPHATAKNGYTPLHIAAKKNQMQIATTLLNYGAETNILTKQGVTPLHLASQGGHTDMVTLLLEKGSNIHVATKTGLTSLHLAAQEDKVNVAEILTKHGANQDAQTKLGYTPLIVACHYGNIKMVNFLLKQGANVNAKTKNGYTPLHQAAQQGHTHIINVLLQHGAKPNAITTNGNTALAIARRLGYISVVDTLKVVTEEITTTTTTVTEKHKLNVPETMTEVLDVSDEEAFKHSDDERFSDGEVYNGTGVISRNSWSDDTMTGDGGEYLRPEDLKELGDDSLPSSQFLDGMNYLRYSLEGGRSDSLRSFSSDRSHTLSHASYLRDSAMIDDTVVIPSQQVTTLAKEAERNSYRLSWGPENLDNVALSSSPIHSGCSSPCLDHDNSSFLVSFMVDARGGAMRGCRHNGLRIIIPPRKCTAPTRVTCRLVKRHRLATMPPMVEGEGLASRLIEVGPSGAQFLGPVIVEIPHFAALRGKERELVILRSENGDSWKEHFCEYTEDELNEILNGMDEVLDTPEELEKKRICRIITRDFPQYFAVVSRIKQDSNLIGPEGGVLSSTVVPQVQAVFPEGALTKRIRVGLQAQPMHTELIKKILGNKATFSPIVTLEPRRRKFHKPITMTIPVPKASSDGIMNGYGGDTPTLRLLCSITGGTTPAQWEDITGTTPLTFVNECVSFTTNVSARFWLIDCRQTQESVTFASQVYREIICVPYMAKFVVFAKSHDPIEARLRCFCMTDDKVDKTLEQQENFAEVARSRDVEVLEGKPIYVDCFGNLVPLTKSGQHHIFSFFAFKENRLPLFVKVRDTTQEPCGRLSFMKEPKSTRGLVHQAICNLNITLPVYTKESESDQEQEEEVDMTSEKNQQDDRERTEERLAHIADHLGFSWTELARELDFTEEQIHQIRIENPNSLQDQSHALLKYWLERDGKHATDTSLTQCLTKINRMDIVHLMETSGIDSMQVHGTRTYTEIEQTIGLDHSEGFSVLQEELYSSRHKPDERHRISKDGDPTEHPPIVSEEDVSVSYSPFQDSTPRSEAELSMAELLRQTHKEQVEAEFSGKPQDVIETTSSQHEYFVTTPGTEQRAASDTSARFSATKEEREKTSPQSPSSAQRGGSPIIQEPEELHLHQDDPSPRRTSLVIVESIDEQPEKLGSGYEEESLEKELAEELGELENSSDEDEMVTTRVVRRRVIIQADSMPEMPPETVTEEQYTDEHGHTVVKKVTRKIIRRYVSPDGTEKEDIIMQGTPQKPVTVEEGDGYSKVVKRVVLKSDSEQSEVTLSEPGVLPSASNFQSEPVEGRKVSKVIKTTVVQGERMEKHLGDASLATDLPSAKEDFEEALSYTGNQIKIQLPALVEKEIMKEDGSIIKRTTLSKASTQKRTVMKDRYGKHVHIEELDDTPEALPQDDLQHDLQQLLRHFCKEDWKQEAK

SEQ ID NO: 160 - protein, 3905 aa, Sus scrofa
EGQSQDKGSKSGSSIQSLFFFSQSDSNASFLRAARAGNLDKVVEYLKGGIDINTCNQNGLNALHLAAKEGHVGLVQELLGRGSSVDSATKKGNTALHIASLAGQAEVVKVLVKEGANINAQSQNGFTPLYMAAQENHIDVVKYLLENGANQSTATEDGFTPLAVALQQGHNQAVAILLENDTKGKVRLPALHIAARKDDTKSAALLLQNDHNADVQSKMMVNRTTESGFTPLHIAAHYGNVNVATLLLNRGAAVDFTARNGITPLHVASKRGNTNMVKLLLDRGGQIDAKTRDGLTPLHCAARSGHDQVVELLLERGAPLLARTKNGLSPLHMAAQGDHVECVKHLLQHKAPVDDVTLDYLTALHVAAHCGHYRVTKLLLDKRANPNARALNGFTPLHIACKKNRIKVMELLVKYGASIQAITESGLTPIHVAAFMGHLNIVLLLLQNGASPDVTNIRGETALHMAARAGQVEVVRCLLRNGALVDARAREEQTPLHIASRLGKTEIVQLLLQHMAHPDAATTNGYTPLHISAREGQVDVASVLLEAGAAHSLATKKGFTPLHVAAKYGSLDVAKLLLQRRAAADSAGKNGLTPLHVAAHYDNQKVALLLLEKGASPHATAKNGYTPLHIAAKKNQMQIASTLLNYGAETNIVTKQGVTPLHLASQEGHTDMVTLLLDKGANIHMSTKSGLTSLHLAAQEDKVNVADILTKHGADQDAHTKLGYTPLIVACHYGNVKMVNFLLKQGANVNAKTKNGYTPLHQAAQQGHTHIINVLLQHGAKPNATTANGNTALAIAKRLGYISVVDTLKVVTEEVTTTTTTITEKHKLNVPETMTEVLDVSDEEGDDTMTGDGGEYLRPEDLKELGDDSLPSSQFLDGMNYLRYSLEGGRSDSLRSFSSDRSHTLSHASYLRDSAMIDDTVVIPSHQVSALAKEAERNSYRLSWGTENLDNVALSSSPIHSGFLVSFMVDARGGAMRGCRHNGLRIIIPPRKCTAPTRVTCRLVKRHRLATMPPMVEGEGLASRLIEVGPSGAQFLGKLHLPTAPPPLNEGESLVSRILQLGPPGTKFLGPVIVEIPHFAALRGKERELVVLRSENGDSWKEHYCEYTEDELNEILNGMDEVLDSPEDLEKKRICRIITRDFPQYFAVVSRIKQDSNLIGPEGGVQAVFPEGALTKRIRVGLQAQPMHSELVKKILGNKATFSPIVTLEPRRRKFHKPITMTIPVPKASSDVMLNGFGGDAPTLRLLCSITGGTTPAQWEDITGTTPLTFVNECVSFTTNVSARFWLIDCRQIQESVTFASQVYREIICVPYMAKFVVFAKSHDPIEARLRCFCMTDDKVDKTLEQQENFAEVARSRDVEVLEGKPIYVDCFGNLVPLTKSGQHHIFSFFAFKENRLPLFVKVRDSTQEPCGRLSFMKEPKSTRGLVHQAICNLNITLPIYTKESESDQEQEEEVIVRHYDETESTETSVLKSHLVNEVPVLASPDLLSEVSEMKQDLIKMTAILTTDVSDRAGSLKVKELVKAAEEEPGEPFEIVERVKEDLEKVNEILRSGTCAGDEGSEPRSQPEREVVEEEWVIVSDEEIEEAKRKAPLEITEYPCVEVRLDKDTKVKVEKDSLGLVNYLTEDLNSYVPPHGEPLQMEREKQEALGPGRSSESEGKDAPSEETQSTQKQPKPSLGIKKPVRRKLKDKQKQKEDSSQASADKSELKKGSSEESLDEDTGLAPEPLPAVKATSPLIEETPIGSIKDKVKALQKRVEDEQKGRSKLPVRVKGKEDVPKKITHRTQLAASPSLKSERHALASKPERHSSLSSPAKTERHPPVSPSSKTEKHSPVSPSAKTERHSPVSSSSKTEKHSPVSPSTKPDRHSPVSSATKTERHPPVSPSGKTDKRPPVSPSGRTEKHPPVSPGRTEKRLPVSPAGRMERHSPMSTSGKTEKHLPVSPSGKTDKQPPVSPTSKTERIEETMSVRELMKAFQSGQDPSKHKTGLFEHKSAKQKQPQEKGKVRGEKEKGLTVTQKETQKTETQTIKRSQRFLVTGPQNPEEQPVVKREEGAGERGKALNHKTPEPVQSAPEEESHKVESPKEKLADEQGDMDLQISPDRKTSTDFSDVIKQELEDNDKYQEFCHNQVLTSPFNTTFPLDYMKEEFLPALSLQSGALDGSSESLKHEGAAGSPCGSLMEGTPQISSEESYKHEGLAETPETSPESLSFSPKKTEEQTEETKETTKVESPPEIHSEKEDPSTKDVTDSSAGQGAVVTGGTEPSAECLLKEATLEPSKDTCPQPEDEGLDSQGESLAKETPKGLTEGVPHGEDPLTHVSSAHEKQTDTEAQKSTASKPSDETAASPLPDAVVKTSPGTESKPQGVIRSPQGLELALPSRDSEVFSPGADESFAVSHKDSLEASPVLEDNSSHKTPDSLEPSPLKESPCRDSLESSPVEPKMKAGILPSQFPLPSAVAKTELITEVASMRSRLLRDPDGSAEDDSLEQTSLMESSGKSPLSPDTPSSEEISYEVTPKTTDAGTPKPAVIPECAEEDDLENGEKKRFTPEEEMFKMVTKIKISKQKRDYKKEPKQEDSSSSSDADVECSADLDEPKCMESVEDKSNAPVVVTAESRKASSSSESEPELTQLKKGADSGLLPEPVIRVQPPSPLPSSIDSNSSPEEVQFQPIVSKQYTFKMNEDTQEDSGKSEEEEGCESHLPEDSHTVSTSGPEMSYDDVNRDADQPKICGSYGCEAESPSSSAIPVTLGLHSSTGEDVHEQLVIHKEPLALQDTGEKDTEGEELDVSRVEAPQVGYPTESSSSSSSLPHCPASEGKELDEDVVSTSSATKMEVTKADQAFESLPDDYSTQDLPNTTQTDSSSLDVPVSDPTETDDISDPQVTSPYENVPSQSFFSSEESKTQTDTRHTSFHSSEVCSVTTTSSVEEVVVTSSSGRTVSSQESNLEDQNLECKQESPLCEVQPDGAPSSLEPAAPTTSTVVGEQISKVIITKTDVDSDSWSEIREDDEAFEARVKEEEQKIFGLMVDRQSQGTTPDTTPARTPTEEGTPTSEQNPFLFQEGKLFEMTRSGAIDMTKRSYGDESFHFFQIGQESREETLTEDMKEGGTGTELPQLETSAESLAPLESKETVNDEADLVPDDLSEEVEEVPTSDGQLNSQMGISASTETSMKEAASAGAEDLPCIQMSDTPSLSLVKQAALQLDFSTVTRSVYSDRDDESPDSSPEEQKSVIEIPTAPMENVPSTESRSKIPVRTMPTSTPAFPSMECESAPSEGFLPSLDEESQEDETKPKSKIPVKAPVPRVEQQLLHLDSSLQETVAPQGQDVTSRAPDSRSKSESDASPLDAKITCPEKTGSYTETDLESSEGTEELELESEDETTRPKIFASRLPVKSKSTTSSCRGATSPTKESKEHFFDLYRNSIEFFEEISDEASKLVDRLTQSEREQELVSDDESSSALEVSVIENLPPVETEQSIPEDIFDTRPIWDESIETLIERIPDENGHDHAEDQQDEQERIEERLAYIADHLGFSWTELARELDFTEEQIHQIRIENPNSLQDQSHALLKYWLERDGKHATDTNLIECLTKINRMDIVHLMETITEPLQERISHSYAEIEQTITLDHSEGFSVLQEELCTAQNKQKEEQAASKEGESYDHPPIVSEEDISVGYSTFQDSIPKTEGDGSVTELLPKTHEEQVQQDFSGKMQDLPEESSLEQQEYFVTTPGTEASETPKATAAPGSPSKTPEEIITPPEEERPYLQTPTASEQGDSPIVQEPEEPPEHREASLPQKTSLVIVESADDQPQTFERLDEDAAFQKGDDMPDIPPETVTEEEYVDEHGHTVVKKVTRKIIRRYVSSDGTEKEEMTMQGAPQDPISIEEGDGYSKVIKRVVLKSDTERSEVRADFVERCK

SEQ ID NO: 161 - protein, 559 aa, Gallus gallus
MDDLDALLADLESTTSHISKRPVFLTEETPYSYPTGNHTYQEIAVPPPVPPPPSSEALNGTVIDPLDQWQPSVSRYGHQQPQSQSPIYSSSAKSSSASVPRDGLSSPSPRASEEEHVYSFPNKQKSAEPSPTMTSTSLGSNLSELDRLLLELNAVQHNPPSGFSADEVSRSPSLPNVTGPHYVIPESSSSAGGKAAPPTKEKPKRNGGRGIEDVRPSVESLLDELESSVPSPVPAITVSQGEVSSPQRVNASQQQTRISASSATRELDELMASLSDFKFMAQGKAGGSSSPPSTTPKPGSQLDTMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGVFFGPEGFHEKDGKAYCRKDYFDMFAPKCGGCARAILENYISALNTLWHPECFVCRECFTPFINGSFFEHDGQPYCEVHYHERRGSLCSGCQKPITGRCITAMGKKFHPEHFVCAFCLKQLNKGTFKEQNDKPYCQNCFLKLFC

SEQ ID NO: 162 - protein, 586 aa, Bos taurus
ALLADLESTTSHISKRPVFLSEETPYSYPTGNHTYQEIAVPPVPPPPSSEALNGSVLDPLDPWPPSTSRFTHQQPQSSSPVYGSSAKTSSASNPQDGGGPPCPRAGEEDHVYSFPNKQKSAEPSPTVMSSSLGSNLSELDRLLLELNAVQHNPPGFPADEANSSPPLPGPLSTHYGVPENNSLLGGKAGALTKEKPKRNGGRGLEDLRPSVENLLDELESSVPSPVPTITVNQGEMSSPQRVTSSQQQTRISASSATRELDELMASLSDLSKIQDLEQRADGELCWAAGWPLNGRQSGPEGQDMGGFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVHGSFFEHEGQPYCEAHYHERRGSLCSGCQKPITGRCITAMAKKFHPEHFVCAFCLKQLNKGTFKEQNDKPYCQNCFLKLFC

SEQ ID NO: 163 - protein, 553 aa, Sus scrofa
DALLADLESTTSHISKRPVFLSEETPYSYPTGNHTYQEIAVPPPVPPPPSSEALNGSVLDPIDQWQPSTSRFIHQQPQAPSPVYGSSAKTSSSSNPQDGIGLPCPRASEEEHVYSFPNKQKSAEPSPTVMSSSLGSNLSELDRLLLELNAVQHNAPGFPTDEANSSPPLPGALSPHYGVLEHNSSLGGKAGPVTKEKPKRNGGRGLEDVRPSVESLLDELESSVPSPVPAITLNQGEMNSPQRVTSSQQQTRISASSATRELDELMASLSDFKFMAQGKTGSSSPPGGPPKPGSQLDSMLGSLQSDLNKLGVATVAKGVCGACKKPIAGQVVTAMGKTWHPEHFVCTHCQEEIGSRNFFERDGQPYCEKDYHNLFSPRCYYCNGPILDKVVTALDRTWHPEHFFCAQCGAFFGPEGFHEKDGKAYCRKDYFDMFAPKCGGCARAILENYISALNTLWHPECFVCRECFTPFVNGSFFEHDGQPYCEVHYHERRGSLCSGCQKPITGRCITAMAKKFHPEHFVCAFCLKQLNKGTFKEQNDKPYCQNCFLKLFC

SEQ ID NO: 164 - protein, 1748 aa, Gallus gallus
MLSMEGFVGARALGEESLQMWDLNKRLEAYLARVKFLEEENEGLRAEIQSTKENPAGDPRRARYEEELRSLRDALHRAFTEKCAAELARDNLYEEVQHVRSRCQKEQAAREEAKRQLSSSKKELEEERRAQIWLKERAVQLEKEVEALLEVHEEEKAGLDQELASFSQSLEGFRCAPVAFQPVEVEDYSKRLSEIWRGAVETYKAEVSQLERALGQAKENLWQVAEDNQQSQLQLRHLEKELVGLKVRKEMLEESLGQQWQEQHGEAEKFQLAIEALEQEKQSLQVQIAQVLEDRQQLMHLKMSLSLEVATYRTLLEAESTRLQMPPGEFKLANSLRDVKLEASSSKHRASLAAFPRPEGVAQLCRTPGDALKVLTPKSKSSSALEFQKISSVLQAPRSWEPAAPSPTVPVVSPEPGSGGAESPVHECGAGKESPMLSPLSPEQLVNHALQDALKEMQDDAEAKEVPTLSATQSTRDGDLEATMEEEEAAGTQGVGAEGETVSPPGLCFCSNEPTLLSATQSDVESQEEMWEEERSKEEMLNPLSSMESQEPGGEPWGGVTRRSRLQVGKEDMEATSTEALHISEKKEQREIWSPSREDEECEFPDEEREMQEEGSLQMEIEAACAVPVGSHPVLPTGIHLQEDFLEREQESEHQETSLGELGAAAGEEREQEVCQELKASSIEEAMPAAEGSSGSGEGTTGRESTGRARDDEGEEEDKGREALGEDDLQAGEALGAKELGKESMGLEEAEGMWEESVDLQEEHRDLQEGHGDLQVEHEDLWEEHGHIQEEHGDTQEEYGDTQEEHGDLQVEGGDLQEEHGDTQEEHGDLQEEHGDTQEEHGDLQVEHEDLQVEHGDLWEEHRDVQEEHGDTQEEYGDTQEEHGDLQVEGGDLQEEHGDTQEEHEDLQEEHGDTQEEHGDLQVEHGDLQEEHRDLQEGHGDLKEEHGDLQVEHEDLQVEHGDLQEEHGDTQEEHGDLQEEHGDLQVEHEDLQVEHGDLQVEHGDLQEEHGDTQEEHRDLQEVHGDQQEEHRDLQEGHGDLQEEHGDLQVEHGDLQEEYGDTQEEHRDLQEVHGDQQEEHRNLQEGHGDLQEGHGDLQEEHGDLWKEHGVLKEEHGDLQEGHGDLQEESGDPQEEPGEPWVQHGEQGSAGDGLEQDMVLQPGEGAWGREDNDISQKQQAQDWEGTAEDEEETGVNTITSQEPTQVDDNPHAEAAENEERDVTSPTAMEETQEGEDEGDAGSEVQSQQQPQDTPGQEAEPAPGQKEVRYGDTGEAPGDPQEPAEALEVEDEELSSEPIELERGSPDTAVMQQDLGNGAESDEPMEEDFQSEDAQLEEPKACRMELEDTLLNSTPLCAYSGEMLESDPNPPSSGGDGEAAPEMAQEEEGDLRGSDEAAVHAEPESCEELSPAPECTEEEEGYFIVSAPSQEGSSMEEAENSEEFEEIKVEAAEDRKDELTAPGVASLVPEDEGHSEPFVGEAEDVKMPLGEFEMPKEEDEEDAGGFAAELEEGLTVPVAEGLSEGHTDKTTLGDEGLGEEDVQDGDNPPATETSDTDPSNTDPPPGTMLEHGAGMEAAEYLPDVPTQLPVDIMKDSDILEIVEQALEFNQELVLGAKLAKDGQEEDGDAQPPQEEEGGSSPTSSCDEQPTVQEAVAEPERTKNGEQNGLHRQASLEDLAEFTEEGLNGITHPGEAPAAHTLPLPSKHSGAEPVPELSPLQTTSCARSRSPGPLGRSDAVGPHSPATIGRLP

SEQ ID NO: 165 - protein, 1483 aa, Danio rerio
MSESLKLQKEQEERDLETRYESSYHQSSLSSVSRQSYTAYVSSHGHKISGKKKEKKLTPLSKKEKRSYLAVDKEDEVIGYVVPVFRSSHLATQDLMETQEEVKEEGVGYVVMRNLFARESGFKMRTVKKTRETSMRESAERMALSQKLHEKEQFQKKMNPDSLTHPPEFIVKPRGQTVWEGKSVTLHCTVAGWPKPRVAWYKNNVLIDAKARPEKYFTESQYNMHSLEIKNCTFSDTAEYRISALNVKGESSAFAPVIIKRFKEEEELVERPHGYSPEYGVTFNTTIIDKFDVSFGREGETMSLGCTVIVYPTVKRYQPEIVWYRNGVALSSSKWVHMHWSGEKATLTLVHLNKEDEGLYTLRVNTKSGFDTHSAYVFVRDADVEEEGVPVAPLDVRCHDANKDYVVVTWKQPAVEGSSAITGYFIDRLEVGNHHWTQCNDTPVKYARFPVTGLIEGRSYMFRVRALNNAGVSRPSRVSDPVVAMDPSDRARLRAGPSAPWTGVIKFTEEDPTVGVIPGPPTDLAVTEATKSYVVLSWKPPVQRGHEGVMYYVEKCLVGTDAWQRVNTGIPVKSPRFALFDLAEDKSYTFRVRSCNSAGVGEPSEETGATTVGDRLDLPSAPGPVIPIRNTDSSVVVCWGASKEVKDLVGYYIEVTVDGSGVWAPCNNKPVKGTRFVCHGLNATDKCTFRVKAVNAAGYSGSSAESEACLVKASIAVPSPPTGVTTLERLRDYMVIGWQAPAKTGGADIRGYYLDYRTVKDGVTSKWHELNLKAVTSSPYKVTDLKENEFYQFQVRAFNQAGISEASIPTAPLECKEWTVAVPGPPHGLRVQEVRKDSMVVLWEPPTFNGRSPVTGYYVDFKEENGRWRCVQERSTKHTYMKVSGLQEGISYRLRIHAKNLAGVGVPSKATDAILAETRPGTNEIVVDVDDNGVISLIFECSDLKEDSQFVWSKNYEAFTDSSRLTIQTTGGKSRAIFNDPSLDDLGIYSCVVTNTDGVSASYTLTEEGLKRLLDISHDHQFPIIPFKSEMAIELQEKGRVRFWAEVGKFTSNLQVEYVFNDNVIHEGKKYTMNFNKSTGIIEMFMDLLEVTDEGTFTFNLVDGKATGRTSLVLIGEEFAELQKKSEFERAEWVRRQGPHFVEYLSFEVTPECDVHLKCKVGNIKPATEIAWFKDGIEIEEDDEDAKKIGKSDEVLTFDIGKLVIKSEKAERKKKPATEESPSKPKISKKDAGVYEVKLKDERGKDKTLLNLTDAGYQAVLNEVFRVIANSSTELKVMSTEHGIILYSFVVHYLEDLRVGWLHKESKISHSDRVQCGVTGEQLWLKINEPTEKDKGKYAIDIFDGKGSVKRVLDLSGQVWEEAFEEFKRLKAAAIAERNRARVVGGLPDVVTIQEGKSLNLTGNVWGDPAPEVSWIKNEKPLVCDEHHTLKYEHSKFASITIAAVTTTDSGKYALLVKNKYGTEAAEFTVSVYIPEDEAEKKE

(19) United States
(12) Patent Application Publication
(10) Pub. No.: US 2025/0151756 A1
(43) Pub. Date: May 15, 2025

(54) COMPOSITIONS AND METHODS FOR PRODUCING FOOD PRODUCTS WITH RECOMBINANT ANIMAL PROTEIN

(71) Applicant: Bond Pet Foods, Inc., Boulder, CO (US)

(72) Inventors: Karin Pernilla Turner Audibert, Golden, CO (US); Anthony George Day, San Francisco, CA (US); Ryan Michael Yamka, Trumbull, CT (US); Luis N. Brandao, Aurora, CO (US)

(21) Appl. No.: 19/030,061

(22) Filed: Jan. 17, 2025

Related U.S. Application Data
(63) Continuation-in-part of application No. 17/427,046, filed on Jul. 29, 2021.
(60) Provisional application No. 62/798,447, filed on Jan. 29, 2019.

(51) Int. Cl. A23K 10/16; A23K 20/147; A23K 50/42; C07K 14/47

(52) U.S. Cl. CPC A23K 10/16; A23K 20/147; A23K 50/42; C07K 14/4716

(57) ABSTRACT

The invention relates to methods and compositions for the recombinant production of animal protein for use in animal food, particularly pet food.

### CROSS REFERENCE TO RELATED APPLICATIONS

This application is a continuation-in-part of U.S. patent application Ser. No. 17/427,046 filed Jul. 29, 2021, which is a U.S. National Phase of International Patent Application No. PCT/US2020/015734 filed Jan. 29, 2020, which claims priority to U.S. Provisional Patent Application No. 62/798,447, filed Jan. 29, 2019, each of which is incorporated herein by reference in its entirety.

### SEQUENCE LISTING

The present application is being filed along with a Sequence Listing in electronic format. The Sequence Listing is provided as a file entitled 68461-702_501_SL.XML, created Jan. 17, 2025, which is 326 kilobytes in size. The information in the electronic format of the Sequence Listing is incorporated by reference in its entirety.

### BACKGROUND OF THE INVENTION

### Field of the invention

The invention relates to methods and compositions for the recombinant production of animal proteins, and also relates to the use of recombinantly produced animal protein in animal food.

### Description of the Related Art

Pets need essential amino acids in the form of protein and these amino acids are often provided by meat from different animals, most often domesticated animals like chicken, cows, and pigs. Another common protein source is fish. Animal meat also contains other nutrients like vitamins, minerals, and fatty acids that are important for the health of pets. The use of meat for feeding pets presents some problems. For instance, animal agriculture is a climate change driver releasing 14.5% of anthropogenic greenhouse gases globally [10]. It also causes biodiversity loss; only 15 mammals and bird species make up over 90% of the livestock production and 60% of mammals are made up by domesticated livestock (on a weight basis), mainly cows and pig, whereas humans account for 36% and wild mammals only 4% [11]. The biomass of poultry is about three times higher than that of wild birds [11]. Populations of vertebrate animals, such as mammals, birds, and fish, have declined by 58% between 1970 and 2012 [12]. Meat also frequently contains pathogens like Salmonella, Shigella, Listeria monocytogenes, Escherichia coli, and Staphylococcus aureus. These pose a risk to both the companion animal and the pet parent [13]. Antibiotic resistance is an immeasurable challenge for humanity and animal agriculture is one of the contributors to the trend [14]. Numerous studies have demonstrated a link between antibiotic use in livestock and increased prevalence of antibiotic-resistant organisms associated with those animals [16]. Of all antibiotics sold in the United States, approximately 80% are sold for use in animal agriculture; about 70% of these are from classes important to human medicine [17].

Although plant sources, e.g., different types of legumes, contain a fair amount of protein, they also contain anti-nutritional factors like fiber, phytate, and protease inhibitors, that limit digestion and absorption [1] [2]. Soybean, a commonly used protein source, decreases the digestibility in canine foods when present in concentrations over 15% [3]. Many plant sources also lack in one or more essential amino acids [4] and thus vegetarian pet foods often contain an inadequate concentration of essential amino acids [5]. In fact, tryptophan and lysine are scarce in corn, lysine in wheat and other cereals, and methionine in soybeans and other legumes [6]. Numerous studies have shown that vegetarian commercial diets for cats and dogs are frequently nutritionally inadequate [7], [8]. Some commercial legume-rich diets may lead to diluted cardiomyopathy in dogs [9]. For all the forgoing reasons, a need exists for improved food for animals that require protein in their diet.

### SUMMARY OF THE INVENTION

In one aspect, provided herein is a pet food or animal feed composition comprising a yeast cell or filamentous fungal cell that expresses at least one recombinant animal protein. In some cases, the composition is substantially free of antibiotics, animal growth hormones, and animal meat. In some cases, the recombinant animal protein has a maximum amino acid sequence identity of 70% to any protein naturally encoded in the genome of the yeast cell or filamentous fungal cell that expresses the recombinant animal protein. In some cases, the recombinant protein is profilin, cofilin, coronin, myozenin, troponin, radixin, myotubularin, gamma-sarcoglycan, alpha-actinin, caveolin, desmin, tropomyosin, titin, connectin, transgelin, smoothelin, actin or myosin. In some cases, the recombinant animal protein is a protein of a chicken, a pig, a turkey, a pheasant, a quail, a horse, cattle, a fish, a shrimp, a prawn, a crab, a sheep, a deer, a duck, a rabbit, an elk, a moose, a kangaroo, an alligator, a wild boar, a goat, a bison, a buffalo, a bear, or an elephant. In some cases, the recombinant animal protein is a poultry protein, even-toed ungulate protein, or fish protein. In some cases, the recombinant animal protein is a cytoskeletal protein. In some cases, the recombinant animal protein is a muscle protein. In some cases, the recombinant animal protein is a skeletal muscle protein. In some cases, the composition comprises a yeast cell or a filamentous fungal cell that expresses at least two different recombinant animal proteins. In some cases, the composition comprises at least two different yeast or filamentous fungal cells that each express at least one different recombinant animal protein. In some cases, the composition comprises about 1% to 30% of the recombinant protein based on the dry weight of the composition. In some cases, the composition comprises about 5% to 40% of the recombinant protein based on the dry weight of the composition. In some cases, the composition further comprises a fat, a carbohydrate, a non-recombinant protein, a fiber, a nutritional supplement, a palatability agent, or any combination thereof. In some cases, the composition comprises 5-50% protein, 0-50% fat, 0-75% carbohydrate, 0-40% dietary fiber, and 0-15% of other nutrients. In some cases, the composition is nutritionally balanced for a companion animal, optionally wherein the composition is a kibble.

In another aspect, provided herein is a method for producing a pet food or animal feed composition according to any one of the preceding, the method comprising culturing yeast or filamentous fungal cells that express an animal protein and processing the cells to provide the pet food or animal feed composition. In some cases, processing the cells comprises combining the cells with a fat, a carbohydrate, a non recombinant protein, a fiber, a nutritional supplement, a palatability agent, or any combination thereof; or dehydrating, baking, and/or extruding the yeast or filamentous fungal cells, optionally combined with a fat, a carbohydrate, a non recombinant protein, a fiber, a nutritional supplement, a palatability agent, or any combination thereof. In some cases, the method comprises lysing the yeast or filamentous fungal cells. In some cases, the method comprises isolating, purifying, and/or concentrating, the animal protein.

Disclosed herein are improved methods and compositions for manufacturing food for animals, particularly companion animals. In certain embodiments, the methods entail producing animal proteins recombinantly in a microbial host, as described herein. The recombinant proteins produced by the method can provide equivalent or better nutrition than conventionally harvested animal proteins or plant-derived proteins, without the associated problems described above. In certain embodiments, the recombinant animal proteins described herein can be incorporated into or serve as food for humans, wild animals and/or zoo animals. In preferred embodiments, the food composition is substantially free of antibiotics, animal growth hormones, and/or meat from farmed, caught or slaughtered animals.

One or a plurality of recombinant proteins can be produced in one organism, or one strain, thereby allowing the amino acid profile to be tailored to the particular nutritional needs of targeted pets and other animals, including humans. Alternatively, a single recombinant animal protein can be produced in one strain (or organism) and mixed with a protein or proteins produced in a different strain (or organism) to yield a final product with the desired proportions of amino acids and other nutrients. Thus, the amino acid profile (and/or the profile of other nutrients) can be customized for the targeted animal, including pets and humans.

### DETAILED DESCRIPTION OF THE INVENTION

### Definitions

Terms used in the claims and specification are defined as set forth below unless otherwise specified.

The term “ameliorating” refers to any therapeutically beneficial result in the treatment of a disease state, e.g., a nutritional deficiency disease state, including prophylaxis, lessening in the severity or progression, remission, or cure thereof.

The term “in situ” refers to processes that occur in a living cell growing separate from a living organism, e.g., growing in tissue culture.

The term “in vivo” refers to processes that occur in a living organism.

The term “mammal” as used herein includes both humans and non-humans and includes but is not limited to humans, non-human primates, canines, felines, murines, bovines, equines, and porcines.

The term percent “identity,” in the context of two or more nucleic acid or polypeptide sequences, refers to two or more sequences or subsequences having a specified percentage of nucleotides or amino acid residues that are identical, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer-based program, sub-sequence coordinates are designated, if necessary, and sequence comparison program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can be a “prophylactically effective amount” as prophylaxis can be considered therapy.

It must be noted that, as used in the specification and the appended claims, the singular forms “a,” “an” and “the” include plural referents unless the context clearly dictates otherwise.

### Compositions of the Invention

### Proteins

The recombinant proteins produced are animal proteins, e.g., proteins from any organ of an animal, including, but not limited to proteins expressed in the brain, skin, scales, feathers, eyes, shells, hair, horns, ears, liver, heart, kidney, stomach, intestines, and muscle tissue. In certain embodiments, the recombinant proteins are animal muscle proteins. In related embodiments, the animal protein is an animal cytoskeletal protein. In other related embodiments, the animal protein is an animal myosin protein or an animal actin protein.

The animal muscle proteins include those proteins normally found in animal muscle tissue (or relatives of those proteins). In addition to myosin and actin, these proteins include troponin, tropomyosin, actinin, titin, connectin, skeletal receptor, myosin-binding protein, desmin, leiomodin, tubulin, myotubularin, myozenin, telethonin, calsarcin, myotilin, nebulin, nebulin-related anchoring protein, myomesin, vinculin, paxillin, beta-enolase, myotubularin, calponin, caldesmon, transgelin, tropomodulin, supervillin, gelsolin, twinfilin, profilin, caveolin, catenin, cofilin, capping protein, leiomodin, tensin, M-protein, radixin, filamin, keratin, myopalladin, calsequestrin, caveolae-associated protein, nebulette, coronin, talin, dystrophin, dystroglycan, integrin, ankyrin, syncoilin, smoothelin-like-1, spectrin, synemin, paranemin, ponsin, plectin, skelemin, sarcoglycan, LIM protein, myoblast determination protein, myocyte-specific enhancer, and myocilin.

The recombinant animal proteins may be full-length proteins, truncated proteins, or fragments of proteins. The sequences of the recombinatly expressed proteins may be modified by replacing one or more amino acids with a different amino acid (e.g., by changing the nucleotide sequence of the recombinant gene encoding the protein). The modifications may improve the yield of protein produced by the organism that has been engineered to express the protein, e.g., by improving the efficiency of transcription and/or translation of the protein, by improving the stability of the protein, by altering the rate at which the protein is secreted by the organism, or by changing the activity of the protein so any deleterious effects of expression of the protein on the recombinant host cell are minimized.

### Protein Origin

The origin of the recombinantly expressed protein sequence (i.e., the species of animal from which the sequence to be recombinantly expressed is found in nature) can be any species within the biological kingdom of Animalia. Preferably, the origin is a vertebrate animal, which can be a fish, bird, mammal, amphibian, or reptile. The origin may be a placental mammal, monotreme mammal, or marsupial mammal (metatheria). The origin may furthermore be a bird or another vertebrate from the reptile clade.

In some embodiments the gene origin is a placental mammal, including but not limited to carnivores (including lion, bear, weasel, seal, wolf, coyote, fox), equidae (including horse and donkey), even-toed ungulates (including pig, camel, cattle, and deer), Afrotheria (including elephants, golden moles, and manatees), and Boreoeutheria (including primates, rabbits, hares, pikas, rodents, moles, whales, bats, dogs, cats, seals, hoofed mammals). In some embodiments the origin is a monotreme mammal, including but not limited to platypus and echidna. In some embodiments the origin is a marsupial mammal, including but not limited to koala, possums, tapirs, kangaroos, wallabies, and marsupial lions. In some embodiments the origin is a hoofed mammal, including but not limited to cattle, antelope, deer, reindeer, elk, sheep, goat, camels, carabao, yak, bison, buffalo, caribou, water buffalo, pig, horse, and donkey. In some embodiments the origin is an endothermic vertebrate, classified as Aves, including but not limited to chicken, turkey, duck, pigeon, penguin, ostrich, goose, pheasant, and quail. In some embodiments the gene origin is a reptile, including but not limited to alligators and crocodiles. In some embodiments the gene origin is an aquatic animal, including but not limited to shark, tuna, trout, salmon, jacks, carp, catfish, cod, flounder, bass, tilapia, sturgeon, crab, lobster, shrimp, prawns, oysters, mussels, eels, shellfish, cuttlefish, starfish, crayfish, and jellyfish. In some embodiments the gene origin is an amphibian, including but not limited to frogs, salamanders, and toads. In some embodiments, the gene origin is an insect.

### Truncated/Mutated Proteins

It is well known that expression of mammalian and avian cytoskeletal proteins, such as actin and tubulin, in microbial hosts can be toxic to those hosts, thus limiting expression levels. This is due to the biological activity of these proteins interfering with the metabolism of the host cell. The nutritional properties of these animal derived proteins will remain essentially unchanged regardless of the biological activity of the protein. Expression levels of toxic recombinant animal proteins can be increased by decreasing their host toxicity via targeted mutagenesis (to decrease the biological activity responsible for the protein's toxicity, for example). Examples are provided herein for improving expression of actin, but these examples should not be construed as limiting. Similar methodology can be used for other proteins.

Preferred protein sequences are listed in Table 1, below. They are grouped according to the tissue in which they are highly expressed (when known). If it is not known in what tissue a protein is expressed, the protein is grouped according to the tissue for which its expression is required (e.g., for normal development of the tissue). For example, it is known that myotubularin is required for normal skeletal muscle growth. Thus, it is grouped with the skeletal muscle proteins. In certain embodiments, the food compositions described herein comprise one or more of the recombinantly produced proteins set forth in Table 1. In related embodiments, the food compositions described herein additionally, or alternatively, comprise one more recombinantly expressed homologs of the recombinantly produced proteins set forth in Table 1. In other related embodiments, the food compositions described herein comprise one more recombinantly expressed proteins that are at least 50%, 60%, 70%, 80%, 85%, 90%, or 95% identical, but less than 100% identical, to the proteins set forth in Table 1 (i.e., the protein sequences are modified to alter their amino acid content, e.g., to improve nutrition, to improve digestibility, to optimize expression or to optimize secretion). In other related embodiments, the food compositions described herein comprise one or more of the recombinantly expressed skeletal muscle tissue proteins of Table 1, or one or more of the recombinantly expressed cardiac muscle tissue proteins of Table 1, or one or more of the recombinantly expressed smooth muscle tissue proteins of Table 1, or one or more of the skeletal/cardiac muscle tissue proteins of Table 1, or one or more of the skeletal/smooth muscle tissue proteins of Table 1, or one or more of the cardiac/smooth muscle tissue proteins of Table 1, or one or more of the skeletal/cardiac/smooth muscle tissue proteins of Table 1. In yet other related embodiments, the food compositions described herein comprise proteins from two or more of the above-mentioned categories of proteins described in Table 1.

### Recombinant Genes Encoding Animal Proteins

Genes encoding recombinant animal proteins can be isolated by taking a sample from an animal and extracting nucleic acids, such as mRNA, from that sample and then amplifying the gene by reverse transcription followed by PCR. The cell sample could be a tissue sample, blood sample, mucus, skin, saliva, or hair. Another option is to have the gene synthesized on demand by a company which performs such work.

The gene sequences (DNA/nucleotide sequences) or protein sequences can be obtained by searching appropriate databases (e.g., UniProtKB and NCBI). A polynucleotide can be obtained using chemical synthesis, molecular cloning or recombinant methods, DNA or gene assembly methods, artificial gene synthesis, PCR, or any combination of those.

The gene sequences are those encoding the types of protein described above. The gene sequences may have non-coding introns.

In the case that there are not sequences available, conserved regions can be used to amplify segments of the genes and the flanking regions can be sequenced in order to obtain the full-length sequence. Multiple sequence alignments of a specific protein in several different organisms will show where the conserved regions lie, and which are the most suitable stretches to use for primer design. Primers with alternative nucleotides can be used when needed.

### Expression Constructs

A genetic element is any coding or non-coding nucleic acid sequence. A genetic element can be a nucleic acid that codes for an amino acid, a peptide or a protein. Genetic elements may be operons, genes, gene fragments, promoters, exons, introns, regulatory sequences, or any combination of those. A genetic element includes an entire open reading frame of a protein, or the entire open reading frame and one or more (or all) regulatory sequences associated therewith. The genes may be codon optimized for different hosts.

The term “engineered” or “recombinant” refers to a cell into which a recombinant gene, such as a gene encoding a muscle protein, or part of a muscle protein, has been introduced. Therefore, engineered cells are distinguishable from naturally occurring cells which do not contain a recombinant gene that is introduced by transfection or transformation techniques. Recombinantly introduced genes will either be in the form of a cDNA (i.e., they will not contain introns), a copy of a cDNA gene, genomic DNA (with or without introns; for expression in prokaryotic hosts, the DNA should be without introns), or will include DNA sequences positioned next to a promoter not naturally associated with the particularly introduced gene.

Disclosed herein are expression vectors comprising a genetic element encoding an animal protein or part of an animal protein and the use thereof for the recombinant expression of the animal protein. The expression vector may further comprise a promoter. The promoter may be a constitutive promoter, an inducible promoter, or a hybrid promoter. In the expression vector, the gene construct and the method, the promoter may be a viral promoter, a prokaryotic promoter or a eukaryotic promoter. The promoter may be a synthetic promoter from a promoter library. The promoter may be any scientifically known promoter or a novel promoter. The promoter may be an engineered form of a known promoter or a hybrid promoter.

The eukaryotic promoter may be a fungi promoter, a plant promoter, or an animal promoter. The fungi promoter may be the promoter of the genes phosphoglycerate kinase (PGK, PGK1, PGK3), enolase (ENO, ENOI), glyceraldehyde-3-phosphate dehydrogenase (gpdA, GAP, GAPDH), hexokinase, pyruvate decarboxylase, phosphofructokinase, glucose-6-phosphate isomerase, 3-phosphoglycerate mutase, pyruvate kinase (pkil), triosephosphate isomerase, phosphoglucose isomerase, glucokinase, alcohol dehydrogenase promoter (ADH1, ADH2, ADH4), isocytochrome C, acidic phosphatase, galactose metabolism enzymesGAL (GAL1, GAL2, GAL3, GAL4, GAL5, GAL6, GAL7, GAL8, GAL9, GAL10), alternative oxidase (AOD), alcohol oxidase I (AOX1), alcohol oxidase 2 (AOX2), CUP1, AHSB4m, adhl+, AINV, alcA, AXDH, cellobiohydrolase I (cbhl), ccg-1, cDNAI, cellular filament polypeptide (cfp), cpc-2, ctr4+, dihydroxyacetone synthase (DAS), FMD, formate dehydrogenase (FMDH), formaldehyde dehydrogenase (FLDI), GAA, GCW14, glucoamylase (glaA, gla-1), invl, isocitrate lyase (ICLI), glycerol kinase (GUTI), acetohydroxy acid isomeroreductase (ILV5), ß-galactosidase (lac4), LEU2, melO, MET3, MET25, KAR2, KEX2, methanol oxidase (MOX), nmtl, peroxin 8 (PEX8), pcbC, PET9, PH05, PH089, PYKI, phosphatidylinositol synthase (PIS1), RPS7, TEF, translation elongation factor 1 alpha (TEFI), sorbitol dehydrogenase (SDH), SSA4, THI11, homoserine kinase, XRP2, TPI, and YPT1, PHO5, CYC1, HIS3, ADC1, TAPI, URA3, LEU2, TP1, TDH1, TDH3, FBA1, ADR1, TPI1, or any combination of those.

The plant promoter may be the promoter of the gene phol, TPI, TPS1, and any combination of these.

The animal promoter may be a heat-shock protein promoter, proactin promoter, immunoglobulin promoter, or the promoter of the gene B2, HSP82, Ser 1, triose phosphate isomerase (TPI1), or any combination of those. However, any promoters can be used if they drive the expression of recombinant proteins in a particular host cell.

The expression vector may include an auxotrophic marker. The auxotrophic marker may be selected from the group consisting of adel, arg4, his4, ura3, and met2. The expression vector may comprise a selectable marker, which may be a resistance gene. The resistance gene may confer resistance to drugs including, but not limited to, zeocin, ampicillin, blasticidin, kanamycin, nurseothricin, chloroamphenicol, tetracycline, triclosan, ganciclovir.

The prokaryotic promoter may be the T7, heat-shock protein (HSP) 70, beta- lactamase, a-amylase, alkaline phosphatase, trp operon, lac operon, tac, trc, gdhA, fic, bgl, xylA, xyIS, AraC, lacSynth, P11, P43, trnQ, sigX, groES, ftsH, and Pm.

In certain embodiments, the compositions of the invention include a host cell transformed with a vector to express one or more recombinant animal muscle proteins. The vector or required genetic segments along with genes of interest may be genomically integrated. Alternatively, or in addition, the host cell may comprise multiple copies of an expression vector. The expression vector may comprise a plasmid. Cells may be transformed by transfection, endocytosis, F-mating, PEG-mediated protoplast fusion, agrobacterium tumefaciens-mediated transformation, chemical transformation, electroporation, or biolistic transformation.

The construct may encode a signal peptide. A signal peptide, also identified as a signal sequence, targeting signal, localization signal, localization sequence, secretion signal, transit peptide, leader sequence, or leader peptide, may cause secretion of a protein. Extracellular secretion of a recombinant or heterologously expressed animal protein from a host cell simplifies protein purification. Recovery of a recombinant animal protein from a cell culture supernatant may be preferable to lysing host cells to release a complex mixture of proteins including intracellular proteins of the host cell. Secretion may reduce harmful effects that intracellular overexpression of a recombinant or heterologous animal protein may have on a host cell such as toxicity or reduced growth rate. Secretion may produce higher amounts of heterologous animal protein compared to intracellular expression. Secretion of a protein may also enable post-translational modification (e.g., glycosylations) or aid in folding the protein correctly and allow for the formation of disulfide bonds.

Food and feed compositions

The recombinant protein composition may be incorporated into food or feed at percentages (based on dry mass) of 0.1-95%, typically between 10% and 90%, more typically between 5% and 50%, including ranges of 5%- 10%, 10-20%, 20-30%, 30-40%, 40-50%, but also including 60-70%, 70-80% and 80%- 90% and combinations of these ranges (e.g., 30%-70%). The recombinant protein compositions can be used in dry or wet food. In preferred embodiments, the end users are companion animals such as dogs and cats. Other users include the animals described herein, including humans.

The recombinant animal protein is typically mixed with other ingredients. The ingredients are any ingredient suitable for consumption by the pet or person. Typical food ingredients include but are not limited to carbohydrates, fats, proteins, fibers, and nutrients such as vitamins, minerals, and trace elements. People skilled in the art can select the amount and type of food ingredients for a typical food based upon the dietary requirements of the pet or person, which can depend on the species, age, size, weight, health, and function.

The food composition can comprise 100% of any particular food ingredient or can comprise a mixture of food ingredients in various proportions. Preferably, the food composition will include, by mass, 5-50% protein, 0.01-1.5% sodium, 0.01-1.5% potassium, 0-50% fat, 0-75% carbohydrate, 0-40% dietary fiber, and 0-15% of other nutrients.

Fat and carbohydrates are obtained from a variety of sources including but not limited to animal fat, fish oil, vegetable oil, meat, meat by-products, grains, other animal or plant sources, and combinations of those. Grains include but are not limited to rice, wheat, corn, barley, buckwheat, sorghum, oats, and quinoa. Other plant sources include but are not limited to pulses (chickpeas and different beans) and edible roots (e.g. potato, sweet potato, carrot, cassava, and turnips).

The supplemental protein food ingredient can be obtained from a variety of sources including plants, animals, or microbes (unicellular and multicellular). Animal protein includes meat, meat by-products, dairy, and eggs. Meats include the flesh from poultry, fish, and animals such as cattle, swine, sheep, goats, and the like. Meat by-products include but are not limited to kidneys, lungs, livers, stomachs, and intestines. The supplemental protein food ingredient may also be free amino acids and/or peptides.

The fiber food ingredient is obtained from a variety of sources such as vegetable fiber sources, including but not limited to beans, cellulose, beet pulp, parsnips, broccoli, peanut hulls, carrots, spinach, and soy fiber.

The nutrients are obtained from a variety of sources known to people skilled in the art, e.g., vitamin and mineral supplements and food ingredients. Vitamins and minerals can be included in amounts required to avoid deficiency and maintain health. These amounts are readily available in the art. The National Research Council (NRC) provides recommended amounts of such nutrients for farm animals. See, e.g., Nutrient Requirements of Swine (11th Rev. Ed., National Academy Press, Wash. D.C., 2012), Nutrient Requirements of Poultry (9th Rev. Ed., National Academy Press, Wash. D.C., 1994), Nutrient Requirements of Horses (6th Rev. Ed., National Academy Press, Wash. D.C., 2007). The American Feed Control Officials (AAFCO) provides recommended amounts of such nutrients for dogs and cats. See American Feed Control Officials, Inc. (Official publication, 2018). Vitamins generally useful as food additives include vitamin A, vitamin B1, vitamin B2, vitamin B6, vitamin B12, vitamin D, biotin, vitamin K, folic acid, inositol, niacin, and pantothenic acid. Minerals and trace elements useful as food additives include calcium, phosphorus, sodium, chloride, potassium, magnesium, iron, copper, zinc, selenium, and iodine. In certain embodiments, the food compositions comprise taurine.

In certain embodiments, the recombinant animal protein is not isolated from the host organism recombinantly expressing the protein prior to mixing with the food composition. The host organism itself, and the recombinant protein expressed within or on its surface, can be added to the food composition.

The food compositions can be in any form useful for feeding the composition to an animal, e.g., kibbles, treats, and toys for animal food. The food compositions can be freeze-dried, dehydrated, air dried, or used as coatings. Kibbles are generally formed using an extrusion process in which the mixture of dry and wet ingredients is mechanically worked at high temperature and pressure and pushed through small openings and cut off into kibble by a rotating knife. The wet kibble is dried and can be coated with one or more topical coatings such as flavors, fats, oils, powders, etc. Kibble also can be made using a baking process when the mix is placed into a mold before dry-heat treatment. Treats may be nutritional and may contain one or more nutrients and may have a food-like composition. Non-nutritional treats are any other treats that are non-toxic. The composition or components are coated onto the treat, incorporated into the treat, or both. Treats of the present invention can be prepared by an extrusion or baking process similar to those used for dry food. Other processes may also be used to either coat the composition on the exterior or inject the composition into an existing treat form. Toys include chewable toys such as artificial bones and food compositions shaped to look like natural foods that are appealing to the animal. The food compositions can be packaged in cans, trays, tubs, pouches, bags, or any other suitable container.

### Pharmaceutical Compositions of the Invention

Methods for treatment of diseases are also encompassed by the present invention. Said methods of the invention include administering a therapeutically effective amount of the recombinant animal protein or a food composition comprising the recombinant animal protein. The recombinant animal proteins of the invention can be formulated in pharmaceutical compositions or therapeutic food compositions. These compositions can comprise, in addition to one or more of the recombinant animal proteins, a pharmaceutically acceptable excipient, carrier, buffer, stabilizer or other materials well known to those skilled in the art. Such materials should be non-toxic and should not interfere with the efficacy of the active ingredient. The precise nature of the carrier or other material can depend on the route of administration, e.g., oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

For treatment purposes, administration of the food composition comprising the recombinantly produced animal protein is preferably in a “therapeutically effective amount” or “prophylactically effective amount” (as the case can be, although prophylaxis can be considered therapy), this being sufficient to show benefit to the animal or individual. The actual amount administered, and rate and time-course of administration, will depend on the nature and severity of protein aggregation disease being treated. Prescription of treatment, e.g., decisions on dosage, etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

A food composition of the invention described herein can be administered alone or in combination with other treatments, either simultaneously or sequentially, dependent upon the condition to be treated.

### Methods of the Invention

In certain embodiments, the invention includes cultivating the host cell and expressing the recombinant animal proteins intracellularly or secreted into the culture broth.

### Harvesting and Processing

Cells expressing recombinant animal protein may be harvested by batch centrifugation, continuous flow centrifugation, filter press, flocculation, rotary drum vacuum filtration, tangential flow filtration, or ultrafiltration.

Cells may be lysed by raising temperature, autolysis, by high pressure homogenization (e.g., French press), ultrasonic cavitation, bead beating, rotor-stator processors, freeze-thaw cycles, enzymatic lysis (e.g., lysozyme, lysostaphin, zymolase, cellulose, protease or glycanase), osmotic shock methods, chemical lysis (by alkaline, detergent or organic solvent) or a combination of these methods. Secreted recombinant animal proteins can be isolated from growth media using well-known techniques.

The whole cell fraction or proteins may be dried by drum drying, spray drying, lyophilization, fluidized bed drying, or other commonly used methods.

### EXAMPLES

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W. H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pennsylvania: Mack Publishing Company, 1990); Carey and Sundberg Advanced Organic Chemistry 3^{rd} Ed. (Plenum Press) Vols A and B (1992). Also referred to below are the following references: (1) M. R. Green and J. Sambrook, Molecular Cloning: A Laboratory Manual, 4th Edition, Cold Spring Harbor, NY: Cold Spring Harbor Laboratory Press, 2012, pp. 1009-1011. (2) G. C. U. F. T. Tool, GenScript, [Online]. Available: https://www.genscript.com/tools/codon-frequency-table. [Accessed 18 12 2018].

(3) S. Wu and L. J. Geoffrey, “High efficiency transformation by electroporation of Pichia pastoris pretreated with lithium acetate and dithiothreitol,” Drug Discovery and genomic technologies, vol. 36, no. 1, pp. 152-154, 2004. (4) S. Kawai, W. Hashimoto and K. Murata, “Transformation of Saccharomyces cerevisiae and other fungi,” Bioengineered Bugs, vol. 1, no. 6, pp. 395-403, 2010. (5) P. Manivasakam and R. H. Schiestl, “High efficiency transformation of Saccharomyces cerevisiae by electroporation,” Nucleic Acids Research, vol. 21, no. 18, pp. 4414-4415, 1993.

### Example 1: Expression of Recombinant Actin Protein in a Eukaryotic host

Actin is the major component of the cytoskeleton. It exists in two different forms, a monomeric form (G-actin) and a filamentous form (F-actin). G-actin polymerizes to form F-actin, and it is primarily these filaments that participate in processes such as cell motility, transport, and cytokinesis [20]. The actin-binding domain is highly conserved amongst species. Actin-binding proteins share a common binding area on the actin surface, consistent of the cleft between actin sub domains 1 and 3 [21]. There is also a nucleotide-binding site, which is a cleft between subdomains 2 and 4. The binding of adenosine 5′-triphosphate or ATP and subsequent hydrolysis into adenosine 5′-diphosphate or ADP is known to be a critical element in controlling the association of actin with itself and with other proteins. When ATP is bound to actin it polymerizes faster and dissociates slower than ADP-actin [22].

Single and double mutants of the ATP-binding site of actin will ablate its toxicity in prokaryotic expression hosts and thus increase expression levels. The residues targeted by mutagenesis are P-72, E-74, I-77, and T-79 (numbering for pig (SEQ P68137), chicken (SEQ P68139), and cow (SEQ P68138)). Recombinant actin protein mutated at these sites will be over-expressed in a prokaryotic host organism, isolated, and incorporated into a companion animal food product.

In one embodiment, then, the invention provides a food composition comprising a recombinant actin protein, wherein said recombinant actin protein comprises one or more mutations from the group consisting of P-72, E-74, I-77 and T-79. In certain related embodiments, the recombinant actin protein is a fragment of actin protein comprising the aforementioned residues.

### Example 2: Identification of Recombinant Actin Sequences for Over-Expression in Prokaryotes

Actin is highly conserved between widely divergent species. For instance, there is 87% sequence identity (325 of 374 amino acids) between yeast and human actin. Comparing chicken, cow, pig, human, and Saccharomyces cerevisiae, there are 319 conserved residues. A library of point mutations is made at each of these conserved positions and those mutations that are permissive of high levels of expression of mutant actin are identified.

### Example 3: Engineered Animal Proteins For Over-Expression in Prokaryotic Cells

Error-prone PCR with/without shuffling will be used across the DNA coding sequence (cDNA) to create mutated DNAs encoding animal protein sequences. Prokaryotic hosts recombinantly expressing the mutant sequences will be screened for high growth and high expression of the target protein.

The genes and the proteins encoded by the genes may also be truncated in order to yield a high expression and fast cell growth. Modifications of the gene sequence (e.g., the addition or removal of certain amino acids) will, in some cases, increase cell viability and increase the rate of cell division. Proteins that are too large to overexpress efficiently will be truncated in order to increase the expression level.

The prokaryotic promoter may be the T7, heat-shock protein (HSP) 70, beta-lactamase, a-amylase, alkaline phosphatase, trp operon, lac operon, tac, trc, gdhA, fic, bgl, xylA, xylS, AraC, lacSynth, P11, P43, trnQ, sigX, groES, ftsH, and Pm.

### Example 5: Insertion of a Chicken Myosin Regulatory Light Chain 2, Skeletal Muscle Isoform, Gene into a Lactococcus lactis Strain and Intracellular Expression of the Corresponding Protein

The expression vector pNZ8152 (MoBiTec GmbH, Gottingen, Germany) is a Gram-positive broad host range vector. Taking advantage of the nisA promoter, it allows intracellular recombinant protein expression via induction with nisin. The vector pNZ8152 also contains the selectable marker alanine racemase gene alr, which restores auxotrophy to D-alanine caused by a deletion of the host cell copy of alr. The vector is linearized using HindIIII restriction enzyme (New England Biolabs, Ipswich, MA) and dephosphorylated using established molecular cloning methods [1]. Linearized vector is separated using agarose gel electrophoresis. An agarose gel section containing linearized vector is collected and the linearized plasmid is purified from the agarose using a commercially available DNA purification kit, e.g. the QIAquick Gel Extraction Kit (Qiagen, Germantown, MD).

The gene sequence for chicken myosin regulatory light chain 2, skeletal muscle isoform, (MYLPF) can be obtained from UniProt.org under accession number P02609. The double-stranded DNA is constructed through chemical gene synthesis from either ATUM (Newark, CA), Genscript (Piscataway, NJ), or IDT (Coralville, Iowa). It is supplied in a vector of choice. The DNA sequence can also be obtained via amplification of cDNA generated directly from a mRNA of a biological sample, such as a tissue or a blood sample from a chicken donor. The gene sequence is modified to aid in cloning, gene expression, or enhance production. It is “codon optimized”, i.e. triplet DNA sequences that are not commonly used in the expression host are changed to those that are commonly used.

The codon optimized myosin regulatory light chain 2, skeletal muscle isoform, gene (MYLPF), containing exons, but no introns, is ligated to the linearized and purified pNZ8152 vector via enzymatic ligation to generate a vector capable of being inserted into a host organism. The method used is known in the art and the protocol can be obtained from a molecular cloning manual [1].

The host organism in this example is Lactococcus lactis strain NZ9130 (MoBiTech). This strain contains genes nisR and nisK, which are needed for nisin-regulated gene expression, as well as a deletion of the alr gene encoding an alanine racemase, causing auxotrophy to D-alanine.

The pNZ8152 vector construct containing the codon optimized myozenin gene is transformed into host strain Lactococcus lactis NZ9130 via electroporation using instrument settings of 2.0 kV, 25 μF, and 200 Q. Electrocompetent cells are obtained via methods known in the art [3]. Chemical transformation or another method can also be used. Successful clones are selected for on agar plates prepared with M17 medium containing 0.5% (w/v) glucose grown at 30°° C. As this medium does not contain D-alanine, only host cells that contain a copy of the plasmid pNZ8152 will be able to proliferate. Colonies are picked and cultured at 30° C. in M17 medium with 0.5% (w/v) glucose for 48 hours in a non-aerated environment. A successful clone containing a copy of the chicken myosin regulatory light chain 2 gene, skeletal muscle isoform, is confirmed by sequencing for insert identity and orientation using established methods such as PCR [1]. Protein expression using the successful clone is induced by culturing in M17 medium with 0.5% (w/v) glucose at 30° C. in a non-aerated environment for 4 hours after cells have reached stationary phase by supplementing 1 ng/ml of nisin. The cells are pelleted by centrifugation and the cell pellets are lysed by methods known in the art, e.g. by sonication [1] and analyzed for protein expression by SDS-PAGE.

Other variants of Lactococcus or Lactobacillus can also be used. The codon usage table is obtained from Kazusa DNA Research Institute.

### Example 6: Insertion of a Chicken Myosin Regulatory Light Chain 2, Skeletal Muscle Isoform, Gene Into a Lactococcus lactis Strain and Extracellular Expression of The Corresponding Protein

The expression vector pNZ8121 (MoBiTec GmbH, Gottingen, Germany) is a Gram-positive broad host range vector. Taking advantage of the nisA promoter, it allows intracellular recombinant protein expression via induction with nisin. It also allows for recombinant protein secretion using the signal sequence PrtP. The vector pNZ8121 also contains the selectable marker conferring resistance to chloramphenicol. The vector is linearized using EcoRV restriction enzyme (New England Biolabs, Ipswich, MA) and dephosphorylated using established molecular scolong methods [1]. Linearized vector is separated using agarose gel electrophoresis. An agarose gel section containing linearized vector is collected and the linearized plasmid is purified from the agarose using a commercially available DNA purification kit, e.g. the QIAquick Gel Extraction Kit (Qiagen, Germantown, MD).

The gene sequence for chicken myosin regulatory light chain 2, skeletal muscle isoform, (MYLPF) can be obtained from UniProt.org under accession number P02609. The double-stranded DNA is constructed through chemical gene synthesis from either ATUM (Newark, CA), Genscript (Piscataway, NJ), or IDT (Coralville, Iowa). It is supplied in a vector of choice. The DNA sequence can also be obtained via amplification of cDNA generated directly from a mRNA of a biological sample, such as a tissue or a blood sample from a chicken donor. The gene sequence is modified to aid in cloning, gene expression, or enhance production. It is “codon optimized”, i.e. triplet DNA sequences that are not commonly used in the expression host are changed to those that are commonly used.

The codon optimized myosin regulatory light chain 2, skeletal muscle isoform, gene (MYLPF), containing exons, but no introns, is ligated to the linearized and purified pNZ8152 vector via enzymatic ligation to generate a vector capable of being inserted into a host organism. The method used is known in the art and the protocol can be obtained from a molecular cloning manual [1].

The host organism in this example is Lactococcus lactis strain NZ9100 (MoBiTech GmbH). This strain contains genes nisR and nisK, which are needed for nisin-regulated gene expression.

The pNZ8121 vector construct containing the codon optimized myozenin gene is transformed into host strain Lactococcus lactis NZ9100 via electroporation using instrument settings of 2.0 kV, 25 μF, and 200 Q. Electrocompetent cells are obtained via methods known in the art [3]. Chemical transformation or another method can also be used. Successful clones are selected for on agar plates prepared with M17 medium containing 0.5% (w/v) glucose and 10 micrograms/mL chloramphenicol grown at 30° C. Colonies are picked and cultured at 30° C. in M17 medium with 0.5% (w/v) glucose and 10 micrograms/mL chloramphenicol for 48 hours in a non-aerated environment. A successful clone containing a copy of the myosin regulatory light chain 2 gene, skeletal muscle isoform, is confirmed by sequencing for insert identity and orientation using established methods such as PCR [1]. Protein expression using the successful clone is induced by culturing in M17 medium with 0.5% (w/v) glucose at 30° C. in a non-aerated environment until stationary phase at which 1 ng/mL of nisin is supplemented for induction. The induction phase is approximately 4 hours. Cultures are clarified via centrifugation and supernatant is analyzed for protein expression by SDS-PAGE.

Other variants of Lactococcus or Lactobacillus can also be used. The codon usage table is obtained from Kazusa DNA Research Institute.

While the invention has been particularly shown and described with reference to a preferred embodiment and various alternate embodiments, it will be understood by persons skilled in the relevant art that various changes in form and details can be made therein without departing from the spirit and scope of the invention.

All references, issued patents and patent applications cited within the body of the instant specification are hereby incorporated by reference in their entirety, for all purposes.

### REFERENCES CITED

[1] H. F. Gemede and N. Ratta, “Antinutritional factors in plant foods: Potential health benefits and adverse effects,” International Journal of Nutrition and Food Sciences, vol. 3, no. 4, pp. 284-289, 2014.
[2] G. Rimbach, J. Pallauf, J. Moehring, K. Kraemer and A. M. Minihane, “Effect of dietary phytate and microbial phyatse on mineral and traceelement bioavailability—a literature review,” Current Topics in Nutraceutical Research, vol. 6, no. 3, pp. 131-144, 2008.
[3] R. M. Yamka, U. Jamikorn, A. D. True and D. L. Harmon, “Evaluation of soyabean meal as a protein source in canine foods,” Animal Feed Science and Technology, vol. 109, pp. 121-132, 2003.
[4] K. E. Michel, “Unconventional Diets for Dogs and Cats,” Veterinary Clinics: Small Animal Practice, vol. 36, no. 6, p. 1269-1281, 2006.

[5] K. Kanakubo, A. J. Fascetti and J. A. Larsen, “Assessment of protein and amino acid concentrations and labeling adequacy of commercial vegetarian diets formulated for dogs and cats,” Journal of the American Veterinary Medical Association, vol. 247, no. 4, pp. 385-392, 2015.
[6] M. Friedman, “Nutritional Value of Proteins from Different Food Sources. A Review,” Journal of Agricultural and Food Chemistry, vol. 44, pp. 6-29, 1996.
[7] C. M. Gray, R. K. Sellon and L. M. Freeman, “Nutritional adequacy of two vegan diets for cats,” Timely Topics in Nutrition, vol. 225, no. 11, pp. 1670-1675, 2004.
[8] A. Knight and M. Leitsberger, “Vegetarian versus Meat-Based Diets for Companion Animals,” Animals, vol. 6, no. 57, pp. 1-20, 2016.

[9] J. L. Kaplan, J. A. Stern, A. J. Fascetti, J. A. Larsen, H. Skolnik, G. D. Peddle, R. D. Kienle, A. C. M. Waxman, C. T. Gunther-Harrington, T. Klose, K. LaFauci and B. Lefbom, “Taurine deficiency and dilated cardiomyopathy in golden retrievers fed commercial diets,” PLOS ONE, 13 December 2018.
[10] M. M. Rojas-Downing, A. P. Nejadhashemi, T. Harrigan and S. A. Woznicki, “Climate change and livestock: Impacts, adaptation, and mitigation,” Climate Risk Management, vol. 16, pp. 145-163, 2017.
[11] Y. M. Bar-On, R. Phillips and R. Milo, “The biomass distribution on Earth,” PNAS, vol. 115, no. 25, pp. 6506-6511, 2018.
[12] “Living Planet Report 2016,” World Wide Fund For Nature, Gland, Switzerland, 2016.
[13] F. P. J. van Bree, G. C. A. M. Bokken, R. Mineur, F. Franssen, M. Opsteegh, J. W. B. van der Giessen, L. J. A. Lipman and P. A. M. Overgaauw, “Zoonotic bacteria and parasites found in raw meat-based diets for cats and dogs,” Veterinary Record, p. 10.1136/vr.104535, 2018.
[14] A. G. Mathew, R. Cissell and S. Liamthong, “Antibiotic Resistance in Bacteria Associated with Food Animals: A United States Perspective of Livestock Production,” FOODBORNE PATHOGENS AND DISEASE, vol. 4, no. 2, pp. 115-133, 2007.
[15] S. A. McEwen and P. J. Fedorka-Cray, “Antimicrobial Use and Resistance in Animals,” Clinical Infectious Diseases, vol. 34, no. Suppl 3, pp. S93-S106, 2002.
[16] W. Witte, “Selective pressure by antibiotic use in livestock,” International Journal of Antimicrobial Agents, vol. 16, pp. S19-S24, 2000.
[17] “Summary Report On Antimicrobials Sold or Distributed for Use in Food-Producing Animals,” Food and Drug Administration, 2014.
[18] J. E. Markovich, C. R. Heinze and L. M. Freeman, “Thiamine deficiency in dogs and cats,” Journal of the American Veterinary Medical Association, vol. 243, no. 5, pp. 649-656, 2013.
[19] M. Steer, “A COMPARISON OF LAND, WATER AND ENERGY USE BETWEEN CONVENTIONAL AND YEAST-DERIVED DAIRY PRODUCTS: AN INITIAL ANALYSIS,” University of the West of England, 2015.
[20] X. Zheng, K. Diraviyam and D. Sept, “Nucleotide Effects on the Structure and Dynamics of Actin,” Biophysical Journal, vol. 93, no. 4, p. 1277-1283, 2007.
[21] R. Dominguez, “A Common Binding Site for Actin-Binding Proteins on the Actin Surface,” in Actin-Monomer-Binding Proteins, New York, NY: Springer, 2007.
[22] T. D. Pollard and G. G. Borisy, “Cellular Motility Driven by Assembly and Disassembly of Actin Filaments,” Cell, vol. 112, no. 4, pp. 453-465, 2003.

TABLE 1
Skeletal muscle tissue
>tr|A4IFM8|A4IFM8_BOVIN Actin, alpha 1, skeletal muscle OS = Bos taurus OX = 9913
GN = ACTA1 PE = 2 SV = 1
MCDEDETTALVCDNGSGLVKAGFAGDDAPRAVFPSIVGRPRHQGVMVGMGQKDSYVGDEAQSKKGILTLKYPIEHGIITNWDDMEKIWHHTFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILTERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVMSGGTTMYPGIADRMQKEITALAPSTMKIKIIAPPERKYSVWIGGSILASLSTFQQMWITKQEYDEAGPSIV
HRKCF (SEQ ID NO: 1)
>sp|P68137|ACTS_PIG Actin, alpha skeletal muscle OS = Sus scrofa OX = 9823 GN = ACTA1 PE = 1
SV = 1
MCDEDETTALVCDNGSGLVKAGFAGDDAPRAVFPSIVGRPRHQGVMVGMGQKDSYVGDEAQSKRGILTLKYPIEHGIITNWDDMEKIWHHTFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILTERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVMSGGTTMYPGIADRMQKEITALAPSTMKIKIIAPPERKYSVWIGGSILASLSTFQQMWITKQEYDEAGPSIV
HRKCF (SEQ ID NO: 2)
>sp|P68139|ACTS_CHICK Actin, alpha skeletal muscle OS = Gallus gallus OX = 9031 GN = ACTA1
PE = 1 SV = 1
MCDEDETTALVCDNGSGLVKAGFAGDDAPRAVFPSIVGRPRHQGVMVGMGQKDSYVGDEAQSKRGILTLKYPIEHGIITNWDDMEKIWHHTFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILTERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVMSGGTTMYPGIADRMQKEITALAPSTMKIKIIAPPERKYSVWIGGSILASLSTFQQMWITKQEYDEAGPSIV
HRKCF (SEQ ID NO: 3)
>sp|P68138|ACTS_BOVIN Actin, alpha skeletal muscle OS = Bos taurus OX = 9913 GN = ACTA1
PE = 1 SV = 1
MCDEDETTALVCDNGSGLVKAGFAGDDAPRAVFPSIVGRPRHQGVMVGMGQKDSYVGDEAQSKRGILTLKYPIEHGIITNWDDMEKIWHHTFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILTERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVMSGGTTMYPGIADRMQKEITALAPSTMKIKIIAPPERKYSVWIGGSILASLSTFQQMWITKQEYDEAGPSIV
HRKCF (SEQ ID NO: 4)
>P02609|MLRS_CHICK Myosin regulatory light chain 2, skeletal muscle isoform
MAPKKAKRRAAEGSSNVFSMFDQTQIQEFKEAFTVIDQNRDGIIDKDDLRETFAAMGRLNVKNEELDAMIKEASGPINFTVFLTMFGEKLKGA
DPEDVIMGAFKVLDPDGKGSIKKSFLEELLTTQCDRFTPEEIKNMWAAFPPDVAGNVDYKNICYVITHGEDKEGE (SEQ ID NO: 5)
>Q9PTY2|Q9PTY2_CHICK Skeletal myosin heavy chain
MSSDAEMAIFGEAAPYLRKSEKERIEAQNKPFDAKTSVFVVHAKESYVKSTIQSKESGKVTVKTESGETLTVKEDQIFSMNPPKY
DKIEDMAMMTHLHEPAVLYNLKERYAAWMIYTYSGLFCVTVNPYKWLPVYNPEVVLAYRGKKRQEAPPHIFSISDNAYQFMLTDRENQSILIT
GESGAGKTVNTKRVIQYFATIAASGDKKKEEQPAGKMQGTLEDQIISANPLLEAFGNAKTVRNDNSSRFGKFIRIHFGATGKLASADIETYLL
EKSRVTFQLKAERSYHIFYQIMSNKKPELIEMLLITTNPYDYHYVSQGEITVPSINDQEELMATDSAIDILGFTPDEKTAIYKLTGAVMHYGN
LKFKQKQREEQAEPDGTEVADKAAYLMGLNSADLLKALCYPRVKVGNEFVTKGQTVQQVYNSVGALAKAVFEKMFLWMVVRINQQLDTKQPRQ
YFIGVLDIAGFEIFDFNSLEQLCINFTNEKLQQFFNHHMFVLEQEEYKKEGIEWEFIDFGMDLAACIELIEKPMGIFSILEEECMFPKATDTS
FKNKLYDQHLGKSNNFQKPKPGKGKAEAHFSLVHYAGTVDYNISGWLDKNKDPLYETVVGLYQKSSLKTLALLFASAGGEAESGGGGKKGGKK
KGSSFQTVSALFRENLNKLMTNLRSTHPHFVRCIIPNETKTPGAMEHELVLHQLRCNGVLEGIRICRKGFPSRILYADFKQRYKVLNASAIPE
GQFIDSKKASEKLLGSIDVDHTQYKFGHTKVFFKAGLLGLLEEMRDEKLAQLITRTQARCRGFLMRVEYRRMVERRESIFCIQYNIRSFMNVK
HWPWMKLFFKIKPLLKSAESEKEMANMKGEFEKTKEELAKSGAKRKDLEGKMVSLLQEKNDLQLQVQAEADALADAEERCDQLIKTKIQLEAK
IKEVTERAEDEEEINAELTAKKRKLEDECSELKKDIDDLELTLAKVEKEKHATENKVKNLTEEMAALDENIAKLTKEKKAPQEAHQQTLDDLQ
AEEDKVNTLTKAKTKLEQQVDDLEGSLEQEKKLRMDLERAKRKLEGDLKLAHDSIMDLENDKQQLDEKLKKKDFEISQIQSKIEDEQALGMQL
QKKIKELQARTEELEEEIEAERTSRAKAEKHRADLSRELEEISERLEEAGGATAAQIDMNKKREAEFQKMRRDLEEATLQHEATAAALRKKHA
DSTAELGEQIDNLQRVKQKLEKEKSELKMEIDDLASNMESVSKAKASLEKTCRALEDQMSEIKTKEEEHQRMINDVNAQRARLQTESGEYSRQ
VEEKDALISQLSRGKQAFTQQIEELKRHLEEEIKAKNALAHGLQSARHDCDLLREQYEEEQEAKGELQRALSKANSEVAQWRTKYETDAIQRT
EELEEAKKKLAQRLQDAEEHVEAVNSKCASLEKTKQRLQNEVEDLMIDVERANSACAALDKKQKNEDKILSEWKQKYEETQAELEASQKESRS
LSTELFKMKNAYEESLDHLETLKRENKNLQQEISDLTEQIAEGGKAIHELEKVKKQIEQEKSELQASLEEAEASLEHEEGKILRLQLELNQVK
SEIDRKIAEKDEEIDQLKRNHLRIVESMQRTLDAEVRSRNEALRLKKKMEGDLNEMEIQLNHANRMAAEAQKNLRNTQGVLKDTQIHLDDALR
SQEDLKEQVAMVERRANLLQAETEELRAALEQTERSRKVAEQELLDASERVQLLHTQNTSLINTKKKLESDISQIQSEMEDTIQEARNAEEKA
KKAITDAAMMAEELKKEQDTSAHLERMKKNLDQTVKDLQHRLDEAEQLALKGGKKQIQKLEARVRELEGEVDAEQKRSAEAVKGVRKYERRVK
ELTYQSEEDRKNVLRLQDLVDKLQMKVKSYKRQAEEAEELSNVNLSKFRKIQHELEEAEERADIAESQVNKLRVKSREFHKKIEEEEI
(SEQ ID NO: 6)
>tr|A0A1S3L3X1|A0A1S3L3X1_SALSA myosin heavy chain, fast skeletal muscle-like isoform X2
OS = Salmo salar OX = 8030 GN = LOC106564162 PE = 3 SV = 1
MSTDAEMQVYGKAAIYLRKSEKERMEAQAMPFDSKNSCYVTDKVELYLKGLVTARADGKCTVTVTKPDGTKEEGKEFKDADIYEMNPPKYDKI
EDMAMMTYLNEASVLYNLKERYAAWMIYTYSGLFCATVNPYKWLPVYDEEVVNAYRGKKRVEAPPHIFSVSDNAFQFMMIDKENQSVLITGES
GAGKTVNTKRVIQYFATIAVSGGKKEADPNKMQGSLEDQIIAANPLLESYGNAKTVRNDNSSRFGKFIRIHFQAGKLAKADIETYLLEKSRVS
FQLPDERGYHIFFQMMTGHKPELVELALLTTNPYDFPMCSQGQIAVASINDNEELDATDEAITILGFTNEEKLGIYKLTGAVVHHGNLKFKQK
QREEQAEPDGTEVADKIAYLLGLNSAEMLKALCYPRVKVGNEYVTKGQTVAQVNNSVSALAKSIYERMFLWMVIRINEMLDTKNPRQFYIGVL
DIAGFEIFDYNSMEQLCINFTNEKLQQFFNHTMFVLEQEEYKKEGIVWAFIDFGMDLAACIELIEKPLGIFSILEEECMFPKSSDTTFKDKLY
AQHLGKTKAFEKPKPAKGKAEAHFSLVHYAGTVDYNITGWLEKNKDPLNDSVCQLYGKSGVKILAALYPPPPPEDKAKKGGKKKGGSMQTVSS
QFRENLHKLMTNLRSTHPHFVRCLIPNESKTPGLMENFLVIHQLRCNGVLEGIRICRKGFPSRIIYADFKQRYKVLNASVIPEGQFMDNKKAS
EKLLGSIDVNHEDYKFGHTKVFFKAGLLGVLEEMRDEKLATLVGMVQALSRGFLMRREFSKMMERRESIYAIQYNIRSFMNVKTWPWMKLYFK
IKPLLQSAETEKELANMKENYEKMKTDLAKALSTKKQMEEKLVSLTQEKNDLALQVASEGESLNDAEERCEGLIKSKIQQEAKLKETTERLED
EEEINAELTAKKRKLEDECSELKKDIDDLELTLAKVEKEKHATENKVKNLTEEMASMDESVAKLTKEKKALQEAHQQTLDDLQAEEDKVNTLT
KAKTKLEQQVDDLEGSLEQEKKLRMDLERAKRKLEGDLKLAQESIMDLENDKQQADEKIKKKEFETTQLLSKIEDEQSLGAQLQKKIKELQAR
IEELEEEIEAERAARAKVEKQRADLSRELEEISERLEEAGGATAAQIEMNKKREAEFQKLRRDLEESTLQHEATAAALRKKQADSVAELGEQI
DNLQRVKQKLEKEKSEYKMEIDDLSSNMEAVAKAKGNLEKMCRTLEDQLSELKTKNDENVRQVNDISGQRARLLTENGEFGRQLEEKEALVSQ
LTRGKQAFTQQVEELKRLIEEEVKAKNALAHGVQSARHDCDLLREQFEEEQEAKAELQRGMSKANSEVAQWRTKYETDAIQRTEELEEAKKKL
AQRLQEAEETIEATNSKCASLEKTKQRLQGEVEDLMIDVERANALAANLDKKQRNFDKVLAEWKQKYEEGQAELEGAQKEARSMSTELFKMKN
SYEEALDHLETLKRENKNLQQEISDLTEQIGETGKSIHELEKAKKTVETEKSEIQTALEEAEGTLEHEESKILRVQLELNQIKGEVDRKIAEK
DEEMEQIKRNSQRVVDSMQSTLDSEVRSRNDALRVKKKMEGDLNEMEIQLSHSNRQAAEAQKQLRNVQGQLKDAQLHLDDAVRAAEDMKEQAA
MVERRNGLMVAEIEELRVALEQTERGRKVAETELVDASERVGLLHSQNTSLLNTKKKLETDLVQVQGEVDDIVQEARNAEEKAKKAITDAAMM
AEELKKEQDTSSHLERMKKNLEVTVKDLQHRLDEAENLAMKGGKKQLQKLESRVRELETEVEAEQRRGVDAVKGVRKYERRVKELTYQTEEDK
KNVNRLQDLVDKLQMKVKAYKRQAEEAEEAANQHMSKFRKVQHELEEAEERADIAETQVNKLRAKTRDSGKGKEAAE (SEQ ID NO: 7)
>tr|A0A1S3QIZ8|A0A1S3QIZ8_SALSA myosin heavy chain, fast skeletal muscle-like QS = Salmo
salar OX = 8030 GN = LOC106593168 PE = 3 SV = 1
MSTDAEMQIYGKAAIYLRKSEKERMEAQAAPFDSKNSCYVADKVELYLKGLITARADGKCTVTVTKPDGTKEEGKEFKDADIYEMNPPKYDKI
EDMAMMTYLNEASVLYNLKERYAAWMIYTYSGLFCATVNPYKWLPVYDAEVVNAYRGKKRMEAPPHIFSVSDNAFQFMLIDKENQSVLITGES
GAGKTVNTKRVIQYFATIAVSGGEKKKEVDPSKMQGSLEDQIIAANPLLEAYGNAKTVRNDNSSRFGKFIRIHFQGGKLAKADIETYLLEKSR
VSFQLPDERGYHIFFQMMTGHKPEIVEMALITTNPYDFPMCSQGQIAVASIDDKEELDATDDAITILGFTNDEKIGIYKLTGAVVHHGNLKFK
QKQREEQAEPDGTEVADKIGYLLGLNSAEMLKALCYPRVKVGNEYVTKGQTVPQVNNSVMALAKSIYERMFLWMVIRINEMLDTKNPRQFYIG
VLDIAGFEIFDYNSMEQLCINFTNEKLQQFFNHTMFVLEQEEYKKEGIVWAFIDFGMDLAACIELIEKPLGIFSILEEECMFPKSSDTTFKDK
LYSQHLGKTQAFEKPKPAKGKAEAHFSLVHYAGTVDYNITGWLEKNKDPLNDSVCQLYGKSGVKILAALYPAAPPEDTTKKGGKKKGGSMQTV
SSQFRENLHKLMTNLRSTHPHFVRCLIPNESKTPGLMENFLVIHQLRCNGVLEGIRICRKGFPSRIIYADFKQRYKVLNASVIPEGQFMDNKK
ASEKLLGSIDVNHEDYKFGHTKVSQILYFKIKPLLQSAETEKELANMKENYEKMTADLAKALSTKKQMEEKLVALMQEKNDLALQVAS
(SEQ ID NO: 8)
>sp|A0JNJ5|MYL1_BOVIN Myosin light chain 1/3, skeletal muscle isoform OS = Bos taurus
OX = 9913 GN = MYL1 PE = 2 SV = 1
MAPKKDVKKPAAAAAPAPAPAPAPAPAPAPPKEEKIDLSAIKIEFSKQQQDEFKEAFLLEDRTGECKITLSQVGDVLRALGINPT
NAEVKKVLGNPSNEEMNAKKIEFEQFLPMLQAISNNKDQGTYEDFVEGLRVFDKEGNGTVMGAELRHVLATLGEKMKEEEVEALMAGQEDSNG
CINYEAFVKHIMSN (SEQ ID NO: 9)
>P02604|MLE1_CHICK Myosin light chain 1, skeletal muscle isoform
MAPKKDVKKPAAAAAPAPAPAPAPAPAPAKPKEPAIDLKSIKIEFSKEQQDDFKEAFLLEDRTGDAKITLSQVGDIVRALGQNPT
NAEINKILGNPSKEEMNAKKITFEEFLPMLQAAANNKDQGTFEDFVEGLRVFDKEGNGTVMGAELRHVLATLGEKMTEEEVEELMKGQEDSNG
CINYEAFVKHIMSV (SEQ ID NO: 10)
>P02605|MLE3_CHICK Myosin light chain 3, skeletal muscle isoform
MSFSPDEINDFKEAFLLFDRTGDAKITLSQVGDIVRALGQNPTNAEINKILGNPSKEEMNAKKITFEEFLPMLQAAANNKDOGTFE
DFVEGLRVFDKEGNGTVMGAELRHVLATLGEKMTEEEVEELMKGQEDSNGCINYEAFVKHIMSV (SEQ ID NO: 11)
>tr|B5DGT2|B5DGT2_SALSA Myosin light chain 3, skeletal muscle isoform OS = Salmo salar
OX = 8030 GN = MLE3 PE = 2 SV = 1
MADAAPAEASGASAFTADQIEDFKEAFGLFDRVGDSMIGYNQVADVMRALGQNPQNKEVAAILGKPSADDMANKRLAFADFMPMMEKVDKIVK
GTLDDYVEGLRVFDKEGNGTVSGAELRIVLGTLGEKMSEAEIDSLLIGQEDENGSINYEAFVKHVLSV (SEQ ID NO: 12)
>sp|P13538|MYSS_CHICK Myosin heavy chain, skeletal muscle, adult OS = Gallus gallus OX = 9031
PE = 1 SV = 4
MASPDAEMAAFGEAAPYLRKSEKERIEAQNKPFDAKSSVFVVHPKESFVKGTIQSKEGGKVTVKTEGGETLTVKEDQVFSMNPPKYDKIEDMA
MMTHLHEPAVLYNLKERYAAWMIYTYSGLFCVTVNPYKWLPVYNPEVVLAYRGKKRQEAPPHIFSISDNAYQFMLTDRENQSILITGESGAGK
TVNTKRVIQYFATIAASGEKKKEEQSGKMQGTLEDQIISANPLLEAFGNAKTVRNDNSSRFGKFIRIHFGATGKLASADIETYLLEKSRVTFQ
LPAERSYHIFYQIMSNKKPELIDMLLITTNPYDYHYVSQGEITVPSIDDQEELMATDSAIDILGFSADEKTAIYKLTGAVMHYGNLKFKQKQR
EEQAEPDGTEVADKAAYLMGLNSAELLKALCYPRVKVGNEFVTKGQTVSQVHNSVGALAKAVYEKMFLWMVIRINQQLDTKQPRQYFIGVLDI
AGFEIFDFNSFEQLCINFTNEKLQQFFNHHMFVLEQEEYKKEGIEWEFIDFGMDLAACIELIEKPMGIFSILEEECMFPKATDTSFKNKLYDQ
HLGKSNNFQKPKPAKGKAEAHFSLVHYAGTVDYNISGWLEKNKDPLNETVIGLYQKSSVKTLALLFATYGGEAEGGGGKKGGKKKGSSFQTVS
ALFRENLNKLMANLRSTHPHFVRCIIPNETKTPGAMEHELVLHQLRCNGVLEGIRICRKGFPSRVLYADFKQRYRVLNASAIPEGQFMDSKKA
SEKLLGSIDVDHTQYRFGHTKVFFKAGLLGLLEEMRDDKLAEIITRTQARCRGFLMRVEYRRMVERRESIFCIQYNVRSFMNVKHWPWMKLFF
KIKPLLKSAESEKEMANMKEEFEKTKEELAKSEAKRKELEEKMVVLLQEKNDLQLQVQAEADSLADAEERCDQLIKTKIQLEAKIKEVTERAE
DEEEINAELTAKKRKLEDECSELKKDIDDLELTLAKVEKEKHATENKVKNLTEEMAVLDETIAKLTKEKKALQEAHQQTLDDLQVEEDKVNTL
TKAKTKLEQQVDDLEGSLEQEKKLRMDLERAKRKLEGDLKLAHDSIMDLENDKQQLDEKLKKKDFEISQIQSKIEDEQALGMQLQKKIKELQA
RIEELEEEIEAERTSRAKAEKHRADLSRELEEISERLEEAGGATAAQIEMNKKREAEFQKMRRDLEEATLQHEATAAALRKKHADSTAELGEQ
IDNLQRVKQKLEKEKSELKMEIDDLASNMESVSKAKANLEKMCRTLEDQLSEIKTKEEQNQRMINDLNTQRARLQTETGEYSRQAEEKDALIS
QLSRGKQGFTQQIEELKRHLEEEIKAKNALAHALQSARHDCELLREQYEEEQEAKGELQRALSKANSEVAQWRTKYETDAIQRTEELEEAKKK
LAQRLQDAEEHVEAVNAKCASLEKTKQRLQNEVEDLMVDVERSNAACAALDKKQKNFDKILAEWKQKYEETQTELEASQKESRSLSTELFKMK
NAYEESLDHLETLKRENKNLQQEIADLTEQIAEGGKAVHELEKVKKHVEQEKSELQASLEEAEASLEHEEGKILRLQLELNQIKSEIDRKIAE
KDEEIDQLKRNHLRIVESMQSTLDAEIRSRNEALRLKKKMEGDLNEMEIQLSHANRMAAEAQKNLRNTQGTLKDTQIHLDDALRTQEDLKEQV
AMVERRANLLQAEVEELRGALEQTERSRKVAEQELLDATERVQLLHTQNTSLINTKKKLETDIVQIQSEMEDTIQEARNAEEKAKKAITDAAM
MAEELKKEQDTSAHLERMKKNMDQTVKDLHVRLDEAEQLALKGGKKQLQKLEARVRELEGEVDSEQKRSAEAVKGVRKYERRVKELTYQCEED
RKNILRLQDLVDKLQMKVKSYKRQAEEAEELSNVNLSKFRKIQHELEEAEERADIAESQVNKLRVKSREIHGKKIEEEE (SEQ ID NO:
13)
>Q8AXY6|MUSK_CHICK Muscle, skeletal receptor tyrosine protein kinase
MRDLLVVPLGHVLTLAALSLAETLQKAPFISTPLETVDALVEDVPKFVCVVESYPEPEITWTRNSIPIRLFDTRYSIQRNGQLLT
ILSVEDSDDGVYCCTADNGVGAAAQSCGALQVKMRPKITRPPVNVEIIEGLKAVLPCTTMGNPKPSVSWIKGETVVKENARIAVLDSGNLRIH
NVQREDAGQYRCVAKNSLGSAYSKPATVVVEVFARILKAPESQNITFGSMVTLRCTAAGAPVPTVTWLENGKAVSAGSIAESVKDRVVDSRLQ
VYVTRPGLFTCLATNKHSKTFGAAKAAATISVSEWSKLYKGDAGYCSTYRGEVCSAILSRNALVFFNSSYADPEETQELLVHTAWTELKTVSS
FCQPAAESLLCNYIFQECKPSGVGPAPKPICRENCLAVKDLYCFKEWLSMEENSQRGIYKPGLMLLALPECNRLPSLHQDPSACTHIPFFDFK
KENITRTCYSGNGQFYQGWANVTASGIPCQKWSDQAPHLHRRTPQVFPELSDAENYCRNPGGENERPWCYTKDPSVTWEYCSVSPCGDASLSL
GTRKPNGETQNLPPPPSYSPTYSMNVIILIISSFALIVILGIITLVCCRRRKQWKNKKRESETPTLTTLPSELLLDRLHPNPMYQRMPLLLNP
KLLSLEYPRNNIEYVRDIGEGAFGRVFQARAPGLLPYEPFTMVAVKMLKEEASADMQADFQREAALMAEFDNPNIVKLLGVCAVGKPMCLLFE
YMAYGDLNEYLRDRSPRNLCSLVQGGLEARACLLNPLALCCTSQLCIAKQVAAGMAYLSERKFVHRDLATRNCLVGENMVVKIADFGLSRNMY
SADYYKANENDAIPIRWMPPESIFYNRYTTESDVWAYGVVLWEIFSYGMQPYYGMAHEEVIYYVRDGNILSCPDNCPLELYNLMRLCWSKLPA
DRPSFASIHRILERMYERAVASPQV (SEQ ID NO: 14)
>P02588|TNNC2_CHICK Troponin C, skeletal muscle
MASMTDQQAEARAFLSEEMIAEFKAAFDMFDADGGGDISTKELGTVMRMLGQNPTKEELDAIIEEVDEDGSGTIDFEEFLVMMVR
QMKEDAKGKSEEELANCFRIFDKNADGFIDIEELGEILRATGEHVTEEDIEDLMKDSDKNNDGRIDFDEFLKMMEGVQ (SEQ ID NO:
15)
>sp|P02587|TNNC2_PIG Troponin C, skeletal muscle OS = Sus scrofa OX = 9823 GN = TNNC2
PE = 1 SV = 2
TDQQAEARSYLSEEMIAEFKAAFDMFDADGGGDISVKELGTVMRMLGQTPTKEELDAIIEEVDEDGSGTIDFEEFLVMMVRQMKEDAKGKSEE
ELAECFRIFDRNMDGYIDAEELAEIFRASGEHVTDEEIESIMKDGDKNNDGRIDFDEFLKMMEGVQ (SEQ ID NO: 16)
>sp|P68246|TNNI2_CHICK Troponin I, fast skeletal muscle OS = Gallus gallus OX = 9031
GN = TNNI2 PE = 1 SV = 2
MSDEEKKRRAATARRQHLKSAMLQLAVTEIEKEAAAKEVEKQNYLAEHCPPLSLPGSMQELQELCKKLHAKIDSVDEERYDTEVKLQKTNKEL
EDLSQKLFDLRGKFKRPPLRRVRMSADAMLRALLGSKHKVNMDLRANLKQVKKEDTEKEKDLRDVGDWRKNIEEKSGMEGRKKMFEAGES
(SEQ ID NO: 17)
>sp|Q8MKI3|TNNT3_BOVIN Troponin T, fast skeletal muscle OS = Bos taurus OX = 9913 GN = Tnnt3
PE = 2 SV = 1
MSDEEVEHVEEEYEEEEEAQEEAPPPPAEVPEVHEEVHEVHEPEEVQEEEKPRPRLTAPKIPEGEKVDFDDIQKKRQNKDLMELQALIDSHFE
ARKKEEEELVALKERIEKRRAERAEQQRIRAEKERERQNRLAEEKARREEEDAKRRAEDDLKKKKALSSMGANYSSYLAKADQKRGKKQTARE
MKKKVLAERRKPLNIDHLSEDKLRDKAKELWDTLYQLETDKFEYGEKLKRQKYDITNLRSRIDQAQKHSKKAGTAPKGKVGGRWK (SEQ ID
NO: 18)
>sp|Q75ZZ6|TNNT1_PIG Troponin T, slow skeletal muscle OS = Sus scrofa OX = 9823 GN = TNNT1
PE = 2 SV = 3
MSDAEEQEYEEEQPEEEEAAEEEEAPEEPEPVAEREEERPKPSRPVVPPLIPPKIPEGERVDFDDIHRKRMEKDLLELQTLIDVHFEQRKKEE
EELVALKERIERRRAERAEQQRFRTEKERERQAKLAEEKMRKEEEEAKKRAEDDAKKKKVLSNMGAHFGGYLVKAEQKRGKRQTGREMKQRIL
SERKKPLNIDHMGEDQLREKAQELSDWIHQLESEKFDLMAKLKQQKYEINVLYNRISHAQKERKGAGKGRVGGRWK (SEQ ID NO: 19)
>NP_990105.1 tropomodulin-4 [Gallusgallus]
MTSYRQELEKYRDIDEDKILQELSAEELEQLDTELLEMDPENVLLPAGLRQRDQTQKSPTGPLDREALLQHLEKQALEAKEREDL
VPFTGEKKGKPFVPKNPTREIPREEQITLEPELEEALANATEAEMCDIAAILGMYTLMSNKQYYDAICSGTITNTEGINSVVKPDKYKPVPDE
PPNPTNVEETLRQIQANDSALEDVNLNNIKDIPISTLKAICEAMKTNTHVKKLSLVATRSNDPVATAVAEMLAENKTLQSLNIESNFITSAGM
MSVIKAMYQNSTLSELKVDNQCQRLGNTVEMEMATMLEQCPSVVRFGYHFTQQGPRARAAIAITRNNELRRKQKKT (SEQ ID NO: 20)
>P68139|ACTS_CHICK Alpha-actin-1
MCDEDETTALVCDNGSGLVKAGFAGDDAPRAVFPSIVGRPRHQGVMVGMGQKDSYVGDEAQSKRGILTLKYPIEHGIITNWDDME
KIWHHTFYNELRVAPEEHPTLLTEAPLNPKANREKMTQIMFETFNVPAMYVAIQAVLSLYASGRTTGIVLDSGDGVTHNVPIYEGYALPHAIM
RLDLAGRDLTDYLMKILTERGYSFVTTAEREIVRDIKEKLCYVALDFENEMATAASSSSLEKSYELPDGQVITIGNERFRCPETLFQPSFIGM
ESAGIHETTYNSIMKCDIDIRKDLYANNVMSGGTTMYPGIADRMQKEITALAPSTMKIKIIAPPERKYSVWIGGSILASLSTFQQMWITKQEY
DEAGPSIVHRKCF (SEQ ID NO: 21)
>P20111|ACTN2_CHICK Alpha-actinin-2
MNSMNQIETNMQYTYNYEEDEYMTQEEEWDRDLLLDPAWEKQQRKTFTAWCNSHLRKAGTQIENIEEDFRNGLKLMLLLEVISGE
RLPKPDRGKMRFHKIANVNKALDYIASKGVKLVSIGAEEIVDGNVKMTLGMIWTIILRFAIQDISVEETSAKEGLLLWCQRKTAPYRNVNIQN
FHLSWKDGLGLCALIHRHRPDLIDYSKLNKDDPIGNINLAMEIAEKHLDIPKMLDAEDIVNTPKPDERAIMTYVSCFYHAFAGAEQAETAANR
ICKVLAVNQENERLMEEYERLASELLEWIRRTIPWLENRTPEKTMQAMQKKLEDFRDYRRKHKPPKVQEKCQLEINFNTLQTKLRISNRPAFM
PSEGKMVSDIAGAWQRLEQAEKGYEEWLLNEIRRLERLEHLAEKFRQKASTHEQWAYGKEQILLQKDYESASLTEVRAMLRKHEAFESDLAAH
QDRVEQIAAIAQELNELDYHDAASVNDRCQKICDQWDSLGTLTQKRREALERTEKLLETIDQLHLEFAKRAAPFNNWMEGAMEDLQDMFIVHS
IEEIQSLISAHDQFKATLPEADGERQAILSIQNEVEKVIQSYSMRISASNPYSTVTVEEIRTKWEKVKQLVPQRDQSLQEELARQHANERLRR
QFAAQANVIGPWIQTKMEEIARSSIEMTGPLEDQMNQLKQYEQNIINYKHNIDKLEGDHQLIQEALVFDNKHTNYTMEHIRVGWELLLTTIAR
TINEVETQILTRDAKGITQEQMNDFRASFNHFDRRKNGLMDHDDFRACLISMGYDLGEAEFARIMSLVDPNGQGTVTFQSFIDEMTRETADTD
TAEQVIASFRILASDKPYILADELRRELPPEQAQYCIKRMPQYTGPGSVPGALDYTSFSSALYGESDL (SEQ ID NO: 22)
>sp|P20111-2|ACTN2_CHICK Isoform 2 of Alpha-actinin-2 OS = Gallus gallus OX = 9031 GN = ACTN2
MNSMNQIETNMQYTYNYEEDEYMTQEEEWDRDLLLDPAWEKQQRKTFTAWCNSHLRKAGTQIENIEEDFRNGLKLMLLLEVISGERLPKPDRG
KMRFHKIANVNKALDYIASKGVKLVSIGAEEIVDGNVKMTLGMIWTIILRFAIQDISVEETSAKEGLLLWCQRKTAPYRNVNIQNFHLSWKDG
LGLCALIHRHRPDLIDYSKLNKDDPIGNINLAMEIAEKHLDIPKMLDAEDIVNTPKPDERAIMTYVSCFYHAFAGAEQAETAANRICKVLAVN
QENERLMEEYERLASELLEWIRRTIPWLENRTPEKTMQAMQKKLEDFRDYRRKHKPPKVQEKCQLEINFNTLQTKLRISNRPAFMPSEGKMVS
DIAGAWQRLEQAEKGYEEWLLNEIRRLERLEHLAEKFRQKASTHEQWAYGKEQILLQKDYESASLTEVRAMLRKHEAFESDLAAHQDRVEQIA
AIAQELNELDYHDAASVNDRCQKICDQWDSLGTLTQKRREALERTEKLLETIDQLHLEFAKRAAPFNNWMEGAMEDLQDMFIVHSIEEIQSLI
SAHDQFKATLPEADGERQAILSIQNEVEKVIQSYSMRISASNPYSTVTVEEIRTKWEKVKQLVPQRDQSLQEELARQHANERLRRQFAAQANV
IGPWIQTKMEEIARSSIEMTGPLEDQMNQLKQYEQNIINYKHNIDKLEGDHQLIQEALVEDNKHTNYTMEHIRVGWELLLTTIARTINEVETQ
ILTRDAKGITQEQMNDFRASFNHFDRRKNGLMDHDDFRACLISMGYDLDESDNLHSDEFKACLISLGEVGNDLQGEAEFARIMSLVDPNGQGT
VTFQSFIDFMTRETADTDTAEQVIASFRILASDKPYILADELRRELPPEQAQYCIKRMPQYTGPGSVPGALDYTSFSSALYGESDL
(SEQ ID NO: 23)
>sp|P13127|CAZA1_CHICK F-actin-capping protein subunit alpha-1 OS = Gallus gallus OX = 9031
GN = CAPZA1 PE = 1 SV = 1
MADFEDRVSDEEKVRIAAKFITHAPPGEFNEVFNDVRLLLNNDNLLREGAAHAFAQYNMDQFTPVKIEGYDDQVLITEHGDLGNGRFLDPRNK
ISFKFDHLRKEASDPQPEDTESALKQWRDACDSALRAYVKDHYPNGFCTVYGKSIDGQQTIIACIESHQFQPKNFWNGRWRSEWKFTITPPTA
QVAAVLKIQVHYYEDGNVQLVSHKDIQDSVQVSSDVQTAKEFIKIIENAENEYQTAISENYQTMSDTTFKALRRQLPVTRTKIDWNKILSYKI
GKEMQNA (SEQ ID NO: 24)
>sp|P28497|CAZA2_CHICK F-actin-capping protein subunit alpha-2 OS = Gallus gallus OX = 9031
GN = CAPZA2 PE = 1 SV = 1
MADLEEQLSDEEKVRIAAKFIIHAPPGEFNEVENDVRLLLNNDNLLREGAAHAFAQYNLDQFTPVKIDGYDEQVLITEHGDLGNGKFLDPKNK
ISFKFDHLRKEATDPRPHEVENAIESWRNSVETAMKAYVKEHYPNGVCTVYGKTIDGQQTIIACIESHQFQAKNFWNGRWRSEWKFTISPSTT
QVAGILKIQVHYYEDGNVQLVSHKDIQDSLTVSNEAQTAKEFIKIVEAAENEYQTAISENYQTMSDTTFKALRRQLPVTRTKIDWNKILSYKI
GKEMQNA (SEQ ID NO: 25)
>tr|A0M8U0|A0M8U0_CHICK Capping protein (Actin filament) muscle Z-line, alpha 2 OS = Gallus
gallus OX = 9031 GN = CAPZA2 PE = 3 SV = 1
MADLEEQLSDEEKVRIAAKFIIHAPPGEFNEVENDVRLLLNNDNLLREGAAHAFAQYNLDQFTPVKIDGYDEQVLITEHGDLGNG
KFLDPKNKISFKFDHLRKEATDPRPHEVENAIESWRNSVETAMKAYVKEHYPNGVCTVYGKTIDGQQTIIACIESHQFQAKNFWNGRWRSEWK
FTISPSTTQVAGILKIQVHYYEDGNVQLVSHKDIQDSLTVSNEAQTAKEFIKIVEAAENEYQTAISENYQTMSDTTFKALRRQLPVTRTKIDW
NKILSYKIGKEMQNA (SEQ ID NO: 26)
>NP_001265047.1 F-actin-capping protein subunit alpha-3 [Gallus gallus]
MSVGQGLCESEKVSLICGLMRQSPPGEFRQVVQDLCDLLQDDELVKQQAARAGARHNKNNFTPVLVNGNTVLLTQYNDLGGNRFFYPQDKFSF
EFDHLSGVTSKTHLHRVMLDEGELWRGALHKGLNAYVNYHFPVGNCCVFKKSLGKRQMLVACIEAHQYQPSKHWNSLWKSDWTFSLTPVMTRV
TGIFLLQLHYFRNANLHVTISKSVSESLHVIDRNQFVTDFVKFVKTEDNKIHNAILENIQALSEHTWRKNLRRRLPITRTFMNWNELLNNQHL
KTGVSRKEVPP (SEQ ID NO: 27)
>P02565|MYH1B_CHICK Myosin-1B
MATDADMAIFGEAAPYLRKSEKERIEAQNKPFDAKSSVFVVHAKESYVKSTIQSKESGKVTVKTEGGETLTVKEDQIFSMNPPKY
DKIEDMAMMTHLHEPAVLYNLKERYAAWMIYTYSGLFCVTVNPYKWLPVYNPEVVLAYRGKKRQEAPPHIFSISDNAYQFMLTDRENQSILIT
GESGAGKTVNTKRVIQYFATIAASGDKKKEEQPAGKMQGTLEDQIISANPLLEAFGNAKTVRNDNSSRFGKFIRIHFGATGKLASADIETYLL
EKSRVTFQLKAERSYHIFYQIMSNKKPELIEMLLITTNPYDYQYVSQGEITVPSINDQEELMATDSAIDILGFTPDEKTAIYKLTGAVMHYGN
LKFKQKQREEQAEPGGTEVADKAAYLMGLNSADLLKALCYPRVKVGNEYVTKGQTVQQVYNSVGALAKSVFEKMFLWMVVRINQQLDTKQPRQ
YFIGVLDIAGFEIFDFNSLEQLCINFTNEKLQQFFNHHMFVLEQEEYKKEGIEWEFIDFGMDLAACIELIEKPMGIFSILEEECMFPKATDTS
FKNKLYDQHLGKSNNFQKPKPGKGKAEAHFSLVHYAGTVDYNITGWLEKNKDPLNETVVGLYQKSSLKTLALLFASVGGAEAESGAGGKKGGK
KKGSSFQTVSALFRENLNKLMSNLRSTHPHFVRCLIPNETKTPGAMEHELVLHQLRCNGVLEGIRICRKGFPIRILYADFKQRYKVLNASAIP
EGQFIDSKKASEKLLGSIDVDHTQYKFGHTKVFFKAGLLGLLEEMRDEKLAQLITRTQARCRGFLMRVEFKKMMERRESIFCIQYNVRAFMNV
KHWPWMKLFFKIKPLLKSAESEKEMANMKEEFEKTKEELAKSEAKRKELEEKMVSLLQEKNDLQLQVQAEADGLADAEERCDQLIKTKIQLEA
KIKELTERAEDEEEMNAELTAKKRKLEDECSELKKDIDDLELTLAKVEKEKHATENKVKNLTEEMAALDETIAKLTKEKKALQEAHQQTLDDL
QAEEDKVNTLTKAKTKLEQQVDDLEGSLEQEKKLRMDLERAKRKLEGDLKMTQESTMDLENDKQQLDEKLKKKDFEISQIQSKIEDEQALGMQ
LQKKIKELQARIEELEEEIEAERTSRAKAEKHRADLSRELEEISERLEEAGGATAAQIDMNKKREAEFQKMRRDLEEATLQHEATAAALRKKH
ADSTADVGEQIDNLQRVKQKLEKEKSELKMEIDDLASNMESVSKAKANLEKMCRSLEDQLSEIKTKEEEQQRTINDISAQKARLQTESGEYSR
QVEEKDALISQLSRGKQAFTQQIEELKRHLEEEIKAKKCPAHALQSARHDCDLLREQYEEEQEAKGELQRALSKANSEVAQWRTKYETDAIQR
TEELEEAKKKLAQRLQDAEEHVEAVNSKCASLEKTKQRLQNEVEDLMIDVERSNAACAALDKKQKNFDKILSEWKQKYEETQAELEASQKESR
SLSTELFKMKNAYEESLDHLETLKRENKNLQQEISDLTEQIAEGGKAIHELEKVKKQIEQEKSELQTALEEAEASLEHEEGKILRVQLELNQV
KSDIDRKIAEKDEEIDQLKRNHLRVVDSMQSTLDAEIRSRNEALRLKKKMEGDLNEIEIQLSHANRQAAEAQKNLRNTQGVLKDTQIHLDDAL
RSQEDLKEQVAMVERRANLLQAEIEELRAALEQTERSRKVAEQELLDASERVQLLHTQNTSLINTKKKLESDISQIQSEMEDTIQEARNAEEK
AKKAITDAAMMAEELKKEQDTSAHLERMKKNLDQTVKDLQHRLDEAEQLALKGGKKQIQKLEARVRELEGEVDAEQKRSAEAVKGVRKYERRV
KELTYQSEEDRKNVLRLQDLVDKLQMKVKSYKRQAEEAEELSNVNLSKFRKIQHELEEAEERADIAESQVNKLRAKSREIGKKAESEE
(SEQ ID NO: 28)
>sp|Q9TV63|MYH2_PIG Myosin-2 OS = Sus scrofa OX = 9823 GN = MYH2 PE = 2 SV = 1
MSSDQEMAIFGEAAPYLRKSEKERIEAQNRPFDAKTSVFVAEPKESFVKGTIQSREGGKVTVKTEAGATLTVKEDQVFPMNPPKFDKIEDMAM
MTHLHEPGVLYNLKERYAAWMIYTYSGLFCVTVNPYKWLPVYNPEVVTAYRGKKRQEAPPHIFSISDNAYQFMLTDRENQSILITGESGAGKT
VNTKRVIQYFATIAVTGEKKKEEPTSGKMQGTLEDQIISANPLLEAFGNAKTVRNDNSSRFGKFIRIHFGTTGKLASADIETYLLEKSRVTFQ
LKAERSYHIFYQITSNRKPELIEMLLITTNPYDYPFISQGEISVASIDDQEELIATDSAIDILGFTNEEKVSIYKLTGAVMHYGNLKFKQKQR
EEQAEPDGTEVADKAAYLQSLNSADLLKALCYPRVKVGNEYVTKGQTVEQVTNAVGALAKAVYEKMFLWMVTRINQQLDTKQPRQYFIGVLDI
AGFEIFDFNSLEQLCINFTNEKLQQFFNHHMFVLEQEEYKREGIEWTFIDFGMDLAACIELIEKPMGIFSILEEECMFPKATDTSFKNKLYEQ
HLGKSANFQKPKPAKGKVEAHFSLIHYAGTVDYNITGWLDKNKDPLNDTVVGLYQKSALKTLAFLFSGAQTGEAEAGGTKKGGKKKGSSFQTV
SALFRENLNKLMTNLRSTHPHFVRCIIPNETKTPGAMEHELVLHQLRCNGVLEGIRICRKGFPSRILYADFKQRYKVLNASAIPEGQYIDSKK
ASEKLLASIDIDHTQYKFGHTKVFFKAGLLGLLEEMRDDKLAQLITRTQARCRGFLARVEYQKMVERRESIFCIQYNIRAFMNVKHWPWMKLF
FKIKPLLKSAESEKEMATMKEEFQKTKDELAKSEAKRKELEEKMVTLLKEKNDLQLQVQAEAEGLADAEERCDQLIKTKIQLEAKIKEVTERA
EDEEEINAELTAKKRKLEDECSELKKDIDDLELTLAKVEKEKHATENKVKNLTEEMAGLDETIAKLTKEKKALQEAHQQTLDDLQAEEDKVNT
LTKAKTKLEQQVDDLEGSLEQEKKLRMDLERAKRKLEGDLKLAQESIMDIENEKQQLDEKLKKKEFEISNLQSKIEDEQALAIQLQKKIKELQ
ARIEELEEEIEAERASRAKAEKQRSDLSRELEEISERLEEAGGATSAQIEMNKKREAEFQKMRRDLEEATLQHEATAAALRKKHADSVAELGE
QIDNLQRVKQKLEKEKSEMKMEIDDLASNMETVSKAKGNLEKMCRTLEDQLSELKSKEEEQQRLINDLTAQRGRLQTESGEFSRQLDEKEALV
SQLSRGKQAYTQQIEELKRQLEEEIKAKNALAHALQSSRHDCDLLREQYEEEQESKAELQRALSKANTEVAQWRTKYETDAIQRTEELEEAKK
KLAQRLQAAEEHVEAVNAKCASLEKTKQRLQNEVEDLMLDVERTNAACAALDKKQRNFDKILAEWKQKYEETHAELEASQKEARSLGTELFKM
KNAYEESLDQLETLKRENKNLQQEISDLTEQIAEGGKRIHELEKIKKQVEQEKSEIQAALEEAEASLEHEEGKILRIQLELNQVKSEVDRKIA
EKDEEIDQLKRNHVRVVESMQSMLDAEIRSRNDAIRLKKKMEGDLNEMEIQLNHANRMAAEALRNYRNTQGILKDTQIHLDDALRGQEDLKEQ
LAMVERRANLLQAEIEELRATLEQTERSRKVAEQELLDASERVQLLHTQNTSLINTKKKLETDISQMQGEMEDILQEARNAEEKAKKAITDAA
MMAEELKKEQDTSAHLERMKKNMEQTVKDLQHRLDEAEQLALKGGKKQIQKLEARVRELEGEVESEQKRNAEAVKGLRKHERRVKELTYQTEE
DRKNILRLQDLVDKLQAKVKSYKRQAEEAEEQSNTNLSKFRKLQHELEEAEERADIAESQVNKLRVKSREVHTKVISEE (SEQ ID NO:
29)
>sp|Q9TV62|MYH4_PIG Myosin-4 OS = Sus scrofa OX = 9823 GN = MYH4 PE = 2 SV = 1
MSSDQEMAIFGEAAPYLRKSEKERIEAQNKPFDAKTSVFVAEPKESFVKGTVQSREGGKVTVKTEAGATLTVKEDQVFPMNPPKFDKIEDMAM
MTHLHEPAVLYNLKERYAAWMIYTYSGLFCVTVNPYKWLPVYNAEVVTAYRGKKRQEAPPHIFSISDNAYQFMLTDRENQSILITGESGAGKT
VNTKRVIQYFATIAVTGEKKKEEPTPGKMQGTLEDQIISANPLLEAFGNAKTVRNDNSSRFGKFIRIHFGTTGKLASADIETYLLEKSRVTFQ
LKAERSYHIFYQIMSNKKPELIEMLLITTNPYDYAFVSQGEITVPSIDDQEELMATDSAIEILGFTSDERVSIYKLTGAVMHYGNLKFKQKQR
EEQAEPDGTEVADKAAYLQGLNSADLLKALCYPRVKVGNEFVTKGQTVQQVYNAVGALAKAVYDKMFLWMVTRINQQLDTKQPRQYFIGVLDI
AGFEIFDFNSLEQLCINFTNEKLQQFFNHHMFVLEQEEYKKEGIEWEFIDFGMDLAACIELIEKPMGIFSILEEECMFPKATDTSFKNKLYEQ
HLGKSNNFQKPKPAKGKAEAHFSLIHYAGTVDYNITGWLDKNKDPLNETVVGLYQKSSVKTLAFLFAERQSSEEGGTKKGGKKKGSSFQTVSA
LFRENLNKLMTNLRSTHPHFVRCIIPNETKTPGAMEHELVLHQLRCNGVLEGIRICRKGFPSRILYADFKQRYKVLNASAIPEGQFIDSKKAS
EKLLGSIDIDHTQYKFGHTKVFFKAGLLGTLEEMRDEKLAQLITRTQAMCRGFLMRVEFRKMMERRESIFCIQYNIRAFMNVKHWPWMKLYFK
IKPLLKSAETEKEMANMKEEFEKTKEDLAKSEAKRKELEEKMVALMQEKNDLQLQVQAEADGLADAEERCDQLIKTKIQLEAKIKEVTERAED
EEEINAELTAKKRKLEDECSELKKDIDDLELTLAKVEKEKHATENKVKNLTEEMAGLDENIAKLTKEKKALQEAHQQTLDDLQAEEDKVNTLT
KAKTKLEQQVDDLEGSLEQEKKLRMDLERAKRKLEGDLKLAQESTMDIENDKQQLDEKLKKKEFEMSNLQSKIEDEQALAMQLQKKIKELQAR
TEELEEEIEAERASRAKAEKQRSDLSRELEEISERLEEAGGATSAQIEMNKKREAEFQKMRRDLEEATLQHEATAAALRKKHADSVAELGEQI
DNLQRVKQKLEKEKSELKMEIDDLASNMETVSKAKGNLEKMCRTLEDQLSEVKTKEEEHQRLINELSAQKARLQTESGEFSRQLDEKEALVSQ
LSRGKQAFTQQIEELKRQLEEETKAKSALAHAVQSSRHDCDLLREQYEEEQEAKAELQRAMSKANSEVAQWRTKYETDAIQRTEELEEAKKKL
AQRLQDAEEHVEAVNAKCASLEKTKQRLQNEVEDLMLDVERSNAACAALDKKQRNFDKILAEWKHKYEETQAELEASQKESRSLSTELFKVKN
AYEESLDQLETLKRENKNLQQEISDLTEQIAEGGKHIHELEKVKKQIEQEKSELQAALEEAEASLEHEEGKILRIQLELNQVKSEIDRKIAEK
DEEIDQMKRNHIRVVESMQSTLDAEIRSRNDALRIKKKMEGDLNEMEIQLNHANRQATEAIRNLRNTQGVLKDTQLHLDDAIRGQDDLKEQLA
MVERRANLMQAEIEELRASLEQTERSRRVAEQELLDASERVQLLHTQNTSLINTKKKLETDISQIQGEMEDIVQEARNAEEKAKKAITDAAMM
AEELKKEQDTSAHLERMKKNMEQTVKDLQHRLDEAEQLALKGGKKQIQKLEARVRELENEVENEQKRNVEAVKGLRKHERRVKELTYQTEEDR
KNVLRLQDLVDKLQSKVKAYKRQAEEAEEQSNVNLSKFRKLQHELEEAEERADIAESQVNKLRVKSREVHTKVISEE (SEQ ID NO: 30)
>sp|P02565|MYH1B_CHICK Myosin-1B OS = Gallus gallus OX = 9031 GN = MYH1B PE = 2 SV = 3
MATDADMAIFGEAAPYLRKSEKERIEAQNKPFDAKSSVFVVHAKESYVKSTIQSKESGhKVTVKTEGGETLTVKEDQIFSMNPPKYDKIEDMA
MMTHLHEPAVLYNLKERYAAWMIYTYSGLFCVTVNPYKWLPVYNPEVVLAYRGKKRQEAPPHIFSISDNAYQFMLTDRENQSILITGESGAGK
TVNTKRVIQYFATIAASGDKKKEEQPAGKMQGTLEDQIISANPLLEAFGNAKTVRNDNSSRFGKFIRIHFGATGKLASADIETYLLEKSRVTF
QLKAERSYHIFYQIMSNKKPELIEMLLITTNPYDYQYVSQGEITVPSINDQEELMATDSAIDILGFTPDEKTAIYKLTGAVMHYGNLKFKQKQ
REEQAEPGGTEVADKAAYLMGLNSADLLKALCYPRVKVGNEYVTKGQTVQQVYNSVGALAKSVFEKMFLWMVVRINQQLDTKQPRQYFIGVLD
IAGFEIFDFNSLEQLCINFTNEKLQQFFNHHMFVLEQEEYKKEGIEWEFIDFGMDLAACIELIEKPMGIFSILEEECMFPKATDTSFKNKLYD
QHLGKSNNFQKPKPGKGKAEAHFSLVHYAGTVDYNITGWLEKNKDPLNETVVGLYQKSSLKTLALLFASVGGAEAESGAGGKKGGKKKGSSFQ
TVSALFRENLNKLMSNLRSTHPHFVRCLIPNETKTPGAMEHELVLHQLRCNGVLEGIRICRKGFPIRILYADFKQRYKVLNASAIPEGQFIDS
KKASEKLLGSIDVDHTQYKFGHTKVFFKAGLLGLLEEMRDEKLAQLITRTQARCRGFLMRVEFKKMMERRESIFCIQYNVRAFMNVKHWPWMK
LFFKIKPLLKSAESEKEMANMKEEFEKTKEELAKSEAKRKELEEKMVSLLQEKNDLQLQVQAEADGLADAEERCDQLIKTKIQLEAKIKELTE
RAEDEEEMNAELTAKKRKLEDECSELKKDIDDLELTLAKVEKEKHATENKVKNLTEEMAALDETIAKLTKEKKALQEAHQQTLDDLQAEEDKV
NTLTKAKTKLEQQVDDLEGSLEQEKKLRMDLERAKRKLEGDLKMTQESTMDLENDKQQLDEKLKKKDFEISQIQSKIEDEQALGMQLQKKIKE
LQARIEELEEEIEAERTSRAKAEKHRADLSRELEEISERLEEAGGATAAQIDMNKKREAEFQKMRRDLEEATLQHEATAAALRKKHADSTADV
GEQIDNLQRVKQKLEKEKSELKMEIDDLASNMESVSKAKANLEKMCRSLEDQLSEIKTKEEEQQRTINDISAQKARLQTESGEYSRQVEEKDA
LISQLSRGKQAFTQQIEELKRHLEEEIKAKKCPAHALQSARHDCDLLREQYEEEQEAKGELQRALSKANSEVAQWRTKYETDAIQRTEELEEA
KKKLAQRLQDAEEHVEAVNSKCASLEKTKQRLQNEVEDLMIDVERSNAACAALDKKQKNFDKILSEWKQKYEETQAELEASQKESRSLSTELF
KMKNAYEESLDHLETLKRENKNLQQEISDLTEQIAEGGKAIHELEKVKKQIEQEKSELQTALEEAEASLEHEEGKILRVQLELNQVKSDIDRK
IAEKDEEIDQLKRNHLRVVDSMQSTLDAEIRSRNEALRLKKKMEGDLNEIEIQLSHANRQAAEAQKNLRNTQGVLKDTQIHLDDALRSQEDLK
EQVAMVERRANLLQAEIEELRAALEQTERSRKVAEQELLDASERVQLLHTQNTSLINTKKKLESDISQIQSEMEDTIQEARNAEEKAKKAITD
AAMMAEELKKEQDTSAHLERMKKNLDQTVKDLQHRLDEAEQLALKGGKKQIQKLEARVRELEGEVDAEQKRSAEAVKGVRKYERRVKELTYQS
EEDRKNVLRLQDLVDKLQMKVKSYKRQAEEAEELSNVNLSKFRKIQHELEEAEERADIAESQVNKLRAKSREIGKKAESEE (SEQ
ID NO: 31)
>sp|Q9TV61|MYH1_PIG Myosin-1 OS = Sus scrofa OX = 9823 GN = MYH1 PE = 2 SV = 1
MSSDQEMAIFGEAAPYLRKSEKERIEAQNKPFDAKTSVFVAEPKESFVKGTVQSREGGKVTVKTEAGATLTVKEDQVFPMNPPKEDKIEDMAM
MTHLHEPAVLYNLKERYAAWMIYTYSGLFCVTVNPYKWLPVYNAEVVTAYRGKKRQEAPPHIFSISDNAYQFMLTDRENQSILITGESGAGKT
VNTKRVIQYFATIAVTGEKKKEEPTSGKMQGTLEDQIISANPLLEAFGNAKTVRNDNSSRFGKFIRIHFGTTGKLASADIETYLLEKSRVTFQ
LKAERSYHIFYQIMSNKKPELIEMLLITTNPYDYAFVSQGEITVPSIDDQEELMATDSAIEILGFTSDERVSIYKLTGAVMHYGNLKFKQKQR
EEQAEPDGTEVADKAAYLQGLNSADLLKALCYPRVKVGNEFVTKGQTVQQVYNAVGALAKAVYDKMFLWMVTRINQQLDTKQPRQYFIGVLDI
AGFEIFDFNSLEQLCINFTNEKLQQFFNHHMFVLEQEEYKKEGIEWEFIDFGMDLAACIELIEKPMGIFSILEEECMFPKATDTSFKNKLYEQ
HLGKSNNFQKPKPAKGKVEAHFSLIHYAGTVDYNITGWLDKNKDPLNETVVGLYQKSSVKTLAFLFTGAAGADAEAGGGKKGGKKKGSSFQTV
SALFRENLNKLMTNLRSTHPHFVRCIIPNETKTPGAMEHELVLHQLRCNGVLEGIRICRKGFPSRILYADFKQRYKVLNASAIPEGQFIDSKK
ASEKLLGSIDIDHTQYKFGHTKVFFKAGLLGLLEEMRDEKLAQLITRTQARCRGFLARVEYQKMVERRESIFCIQYNIRAFMNVKHWPWMKLY
FKIKPLLKSAETEKEMANMKEEFEKTKESLAKAEAKRKELEEKMVALMQEKNDLQLQVQAEADSLADAEERCDQLIKTKIQLEAKIKEVTERA
EDEEEINAELTAKKRKLEDECSELKKDIDDLELTLAKVEKEKHATENKVKNLTEEMAGLDETIAKLTKEKKALQEAHQQTLDDLQAEEDKVNT
LTKAKTKLEQQVDDLEGSLEQEKKLRMDLERAKRKLEGDLKLAQESTMDIENDKQQLDEKLKKKEFEMSNLQSKIEDEQALAMQLQKKIKELQ
ARIEELEEEIEAERASRAKAEKQRSDLSRELEEISERLEEAGGATSAQIEMNKKREAEFQKMRRDLEEATLQHEATAATLRKKHADSVAELGE
QIDNLQRVKQKLEKEKSEMKMEIDDLASNMETVSKAKGNLEKMCRTLEDQLSELKTKEEEQQRLINDLTAQRARLQTESGEYSRQLDEKDTLV
SQLSRGKQAFTQQIEELKRQLEEEIKAKSALAHAVQSSRHDCDLLREQYEEEQEAKAELQRAMSKANSEVAQWRTKYETDAIQRTEELEEAKK
KLAQRLQDAEEHVEAVNAKCASLEKTKQRLQNEVEDLMIDVERSNAACAALDKKQRNFDKILAEWKQKYEETHAELEASQKESRSLSTELFKV
KNAYEESLDQLETLKRENKNLQQEISDLTEQIAEGGKRIHELEKIKKQVEQEKSEIQAALEEAEASLEHEEGKILRIQLELNQVKSEVDRKIA
EKDEEIDQLKRNHVRVVESMQSMLDAEIRSRNDAIRLKKKMEGDLNEMEIQLNHANRMAAEALRNYRNTQGILKDTQIHLDDALRSQEDLKEQ
LAMVERRANLLQAEIEELRATLEQTERSRKVAEQELLDASERVQLLHTQNTSLINTKKKLETDISQIQGEMEDIIQEARNAEEKAKKAITDAA
MMAEELKKEQDTSAHLERMKKNLEQTVKDLQHRLDEAEQLALKGGKKQIQKLEARVRELEGEVESEQKRNVETVKGLRKHERRVKELTYQTEE
DRKNILRLQDLVDKLQAKVKSYKRQAEEAEEQSNVNLSKFRKLQHELEEAEERADIAESQVNKLRVKSREVHTKIISEE (SEQ ID NO:
32)
>tr|Q9DGM4|Q9DGM4_CHICK Fast myosin heavy chain isoform 3 OS = Gallus gallus OX = 9031
PE = 2 SV = 1
MASSDAEMAAFGEAAPYHRKSEKERIEAQNKPFDAKSSVFVAHPKESFVKGTIQSRETGKVTVKTEGGETLTVKEDQVFSMNPPKYDKIEDMA
MMTHLHEPAVLYNLKERYAAWMIYTYSGLFCVTVNPYKWLPVYNPEVVLAYRGKKRQEAPPHIFSISDNAYQFMLTDRENQSILITGESGAGK
TVNTKRVIQYFATIAASGEKKKEEQSGKMQGTLEDQIISANPLLEAFGNAETVRNDNSSRFGKFIRIHFGATGKLASADIETYLLEKSRVTFQ
LKAERSYHIFYQIMSNKKPELIDMLLITTNPYDYHFVSQGEITVPSIDDQEELMATDSAIDILGFTADEKTAISKLTGAVMHYGNLKFKQKQR
EEQAEPDGTEVADKAAYLMGLNSADLLKALCYPRVKVGNEYVTKGQTVQQVHNAVGALAKAVYEKMFLWMVVRINQQLDTKQPRQYFIGVLDI
AGFEIFDFNSFEQLCINFTNEKLQQFFNHHMFVLEQEEYKKEGIEWTFIDFGMDLAACIELIEKPMGIFSILEEECMFPKATDTSFKNKLYDQ
HLGKSSNFQKPKPAKGKAEAHFSLVHYAGTVDYNITGWLEKNKDPLNETVIGLYQKSSVKTLALLFATYGGADAEAGGGGKKGGKKKGSSFQT
VSALFRENLNKLMTNLRSTHPHFVRCIIPNETKTPGAMEHELVLHQLRCNGVLEGIRICRKGFPSRVLYADFKQRYKVLNASAIPEGQFIDSK
KASEKLLSSIDVDHTQYKFGHTKVFFKAGLLGLLEEMRDEKLAQLITRTQARSRGFLMRVEYQRMVERRESIFCIQYNVRSFMNVKHWPWMKL
FFKIKPLLKSAESEKEMANMKEEFEKTKEELAKSEAKRKELEEKMVKLVQEKNDLQLQVQAEADSLADAEERCDQLIKTKIQLEAKIKEVTER
AEDEEEINAELTAKKRKLEDECSELKKDMDDLELTLAKVEKEKHATENKVKNLTEEMAALDETIVKLTKEKKALQEAHQQTLDDLQAEEDKVN
TLTKAKTKLEQQVDDLEGSLEQEKKLRMDLERAKRKLEGDLKLAHDSIMDLENDKQQLDEKLKKKDFEISQIQSKIEDEQALGMQLQKKIKEL
QARIEELEEEIEAERTSRAKAEKHRADLSRELEEISERLEEAGGATAAQIDMNKKREAEFQKMRRDLEEATLQHEATAAALRKKHADSTAELG
EQIDNLQRVKQKLEKEKSELKMEIDDLASNMESVSKAKANLEKMCRTLEDQLSEIKTKEEEHQRMINDLNTQRARLQTEAGEYSRQVEEKDAL
ISQLSRGKQAFTQQIEELKRHLEEEIKAKNALAHALQSARHDCDLLREQYEEEQEAKGELQRALSKANSEVAQWRTKYETDAIQRTEELEEAK
KKLAQRLQDAEEHVEAVNAKCASLEKTKQRLQNEVEDLMIDVERANAACAALDKKQKNFDKILAEWKQKYEETQAELEASQKESRSLSTELFK
MKNAYEESLDHLQTLKRENKNLQQEISDLTEQIAEGGKAIHELEKVKKQIEQEKSEIQAALEEAEASLEHEEGKILRLQLELNQVKSEIDRKI
AEKDEEIDQLKRNHLRIVESLQSSLDAEIRSRNEALRLKKKMEGDLNEMEIQLSHANRVAAEAQKNLRNTQAVLKDTQIHLDDALRTQEVLKE
QVAMVERRANLLQAEIEELRAALEQTERSRKVAEQELMDASERVQLLHTQNTSLINTKKKLETDIAQIQSEMEDTIQEARNTEEKAKKAITDA
AMMAEELKKEQDTSAHLERMKKNLDQTVKDLQHRLDEAEQLALKGGKKQIQKLEARVRELEGEVDAEQKRSAEAVKGVRKYERRVKELTYQSE
EDLKNILRLQDLVDKLQMKVKSYKRQAEEAEELSNVNLSKFRKIQHELEEAEERADIAESQVNKLRVKSREFHSKKIEEEE (SEQ ID NO:
33)
>P13538|MYSS_CHICK
MASPDAEMAAFGEAAPYLRKSEKERIEAQNKPFDAKSSVFVVHPKESFVKGTIQSKEGGKVTVKTEGGETLTVKEDQVFSMNPPK
YDKIEDMAMMTHLHEPAVLYNLKERYAAWMIYTYSGLFCVTVNPYKWLPVYNPEVVLAYRGKKRQEAPPHIFSISDNAYQFMLTDRENQSILI
TGESGAGKTVNTKRVIQYFATIAASGEKKKEEQSGKMQGTLEDQIISANPLLEAFGNAKTVRNDNSSRFGKFIRIHFGATGKLASADIETYLL
EKSRVTFQLPAERSYHIFYQIMSNKKPELIDMLLITTNPYDYHYVSQGEITVPSIDDQEELMATDSAIDILGFSADEKTAIYKLTGAVMHYGN
LKFKQKQREEQAEPDGTEVADKAAYLMGLNSAELLKALCYPRVKVGNEFVTKGQTVSQVHNSVGALAKAVYEKMFLWMVIRINQQLDTKQPRQ
YFIGVLDIAGFEIFDFNSFEQLCINFTNEKLQQFFNHHMFVLEQEEYKKEGIEWEFIDFGMDLAACIELIEKPMGIFSILEEECMFPKATDTS
FKNKLYDQHLGKSNNFQKPKPAKGKAEAHFSLVHYAGTVDYNISGWLEKNKDPLNETVIGLYQKSSVKTLALLFATYGGEAEGGGGKKGGKKK
GSSFQTVSALFRENLNKLMANLRSTHPHFVRCIIPNETKTPGAMEHELVLHQLRCNGVLEGIRICRKGFPSRVLYADFKQRYRVLNASAIPEG
QFMDSKKASEKLLGSIDVDHTQYRFGHTKVFFKAGLLGLLEEMRDDKLAEIITRTQARCRGFLMRVEYRRMVERRESIFCIQYNVRSFMNVKH
WPWMKLFFKIKPLLKSAESEKEMANMKEEFEKTKEELAKSEAKRKELEEKMVVLLQEKNDLQLQVQAEADSLADAEERCDQLIKTKIQLEAKI
KEVTERAEDEEEINAELTAKKRKLEDECSELKKDIDDLELTLAKVEKEKHATENKVKNLTEEMAVLDETIAKLTKEKKALQEAHQQTLDDLQV
EEDKVNTLTKAKTKLEQQVDDLEGSLEQEKKLRMDLERAKRKLEGDLKLAHDSIMDLENDKQQLDEKLKKKDFEISQIQSKIEDEQALGMQLQ
KKIKELQARIEELEEEIEAERTSRAKAEKHRADLSRELEEISERLEEAGGATAAQIEMNKKREAEFQKMRRDLEEATLQHEATAAALRKKHAD
STAELGEQIDNLQRVKQKLEKEKSELKMEIDDLASNMESVSKAKANLEKMCRTLEDQLSEIKTKEEQNQRMINDLNTQRARLQTETGEYSRQA
EEKDALISQLSRGKQGFTQQIEELKRHLEEEIKAKNALAHALQSARHDCELLREQYEEEQEAKGELQRALSKANSEVAQWRTKYETDAIQRTE
ELEEAKKKLAQRLQDAEEHVEAVNAKCASLEKTKQRLQNEVEDLMVDVERSNAACAALDKKQKNEDKILAEWKQKYEETQTELEASQKESRSL
STELFKMKNAYEESLDHLETLKRENKNLQQEIADLTEQIAEGGKAVHELEKVKKHVEQEKSELQASLEEAEASLEHEEGKILRLQLELNQIKS
EIDRKIAEKDEEIDQLKRNHLRIVESMQSTLDAEIRSRNEALRLKKKMEGDLNEMEIQLSHANRMAAEAQKNLRNTQGTLKDTQIHLDDALRT
QEDLKEQVAMVERRANLLQAEVEELRGALEQTERSRKVAEQELLDATERVQLLHTQNTSLINTKKKLETDIVQIQSEMEDTIQEARNAEEKAK
KAITDAAMMAEELKKEQDTSAHLERMKKNMDQTVKDLHVRLDEAEQLALKGGKKQLQKLEARVRELEGEVDSEQKRSAEAVKGVRKYERRVKE
LTYQCEEDRKNILRLQDLVDKLQMKVKSYKRQAEEAEELSNVNLSKERKIQHELEEAEERADIAESQVNKLRVKSREIHGKKIEEEE
(SEQ ID NO: 34)
>sp|P16419|MYPC2_CHICK Myosin-binding protein C, fast-type OS = Gallus gallus OX = 9031
GN = MYBPC2 PE = 1 SV = 3
MPEPSKAAPKKEAKKKEEKKEEKKEAPPPQEHKDEAPDDVHPPETPDPEGLFLSKPQNVMVESGRDVTVSARVAGAALPCAPAVK
WFKGKWAELGDKSARCRLRHSVDDDKVHTFELTITKVAMGDRGDYRCEVTAKEQKDSCSFSIDVEAPRSSEGNVLQAFKRTGEGKDDTAGELD
FSGLLKKREVQVEEKKKKKDEDDQFPPEIWELLKGVTKKSEYERIAFQYGITDLRGMLKRLKKVHVEPKKSEAFIRKLDPAYQVDKGNKIKLV
VELSDPDLPLKWYKNGQLLKPSTKYVFENVGLKRILTIHKCSLADDAAYECRVNDEKCFTEVFVKEPPVTVVRGLEDQQVVVGDRVVLEAEVS
EEGAQVMWLKDGVDVTRDDAFKYRFKKDGKKHFLIINEAELSDSAHYKIMTNGGESEAELSVEEKQLEVLQDMADLTVKASEQAVFKCEVSDE
KVTGRWFRNGVEVKPSKRIHISHNGRFHKLVIDDVRPEDEGDYTFIPDGYALSLSAKLNFLEIKVEYVPKQEPPKIHLDCSGKAAENTIVVVA
GNKVRLDVPISGEPAPTVTWKRGDQLFTATEGRVHIDSQADLSSFVIESAERSDEGRYCITVTNPVGEDSATLHVRVVDVPDPPQSVRVTSVG
EDWAVLSWEAPPFDGGMPITGYLMERKKKGSMRWMKLNFEVFPDTTYESTKMIEGVFYEMRVFAVNAIGVSQPSLNTQPFMPIAPTSEPTHVV
LEDVTDTTATIKWRPPERIGAGGVDGYLVEWCREGSNEWVAANTELVERCGLTARGLPTGERLLFRVISVNMAGKSPPATMAQPVTIREIVER
PKIRLPRHLRQTYIRRVGEQVNLVIPFQGKPRPQVTWSREGGALPAEVQTRTSDVDSVFFIRSAARPLSGNYEMRVRIDNMEDCATLRLRVVE
RPGPPQAVRVMEVWGSNALLQWEPPKDDGNAEISGYTVQKADTRTMEWFTVLEHSRPTRCTVSELVMGNEYRFRVYSENVCGTSQEPATSHNT
ARIAKEGLTLKMVPYKERDLRAAPQFLTPLVDRSVVAGYTVTLNCAVRGHPKPKVTWLKNSVEIGADPKFLSRHGLGVLSLLIRRPGPFDGGT
YGCRAVNEMGEATTECRLDVRVPQ (SEQ ID NO: 35)
>tr|E1BNV1|E1BNV1_BOVIN Myosin binding protein C, fast type OS = Bos taurus OX = 9913
GN = MYBPC2 PE = 4 SV = 2
MPEAKPAAKKAPAGKDAAAKPAPKEATPQEAPAAPPTEAPPEDQSPTAEEPTGVFLKKPDSVSVETGKDTVIVAKLNGKELPAKPAVKWFKGK
WLELGSKSGARFSFKESHDAASNVYTIELHITKVVLGDRGDYRIEVKAKDFCDSCAFNIDVEAPRQDSAGQSLESFKRSGEAKSDTAGELDES
GLLKKRQVVEEEKKKKKDDDDLGIPPEIWELLKGAKKSEYERIAFQYGITDLRGMLKRLKKAKVEVKKSAAFTKKLDPAYQVDRGNKIKLVVE
ISDPDLPLKWFKNGQEIKPSSKYVFENVGKKRILTINKCTLADDAAYEVVVKDEKCFTELFVKVEPPVLIVTPLEDQQVFVGDRVEMAVEVSE
EGAQVMWLKDGVELTREDSFKARYRLKKDGKRHILIYSEVTMEDKGHYQVMTNGGQCEAELIVEEKQLEVLQDIADLTVKASEQAVFKCEVSD
EKVTGKWYKNGVEVRPSKRITISHTGRFHKLVIDDVRPEDEGDYTFVPDGYALSLSAKLKFLEIKVEYVPKQEPPKIHLDCSGQTSENAIVVV
AGNKLRMDVSITGEPRPVATWMKEDEVFTGTEGRVRIEQRGDISSEVIESAERGDEGRYTIKVTNPVGEDVASILLKVVDVPDPPEAVRVTSV
GEDWAVLVWEPPKYDGGRPVTGYLLERKKKGSQRWMKLNFEVFTETTYESTNMIEGILYEMRVFAAPLIQQLKTLTPQPHAPFSPTSEPQHLT
VEDVTDTTTTLKWRPPDKIGAGGIDGYLIEYCVEGSDEWIPANTEPVERCGFTVKNLPTGAKITFRVVGVNIAGRSQPATLAQPVTIREIVEQ
PKIRLPRHLRQTYVRKVGEHLNLVIPFQVRRAKGGAPIDTSHVHVRTSDFDTVFFVRQAARSDSGEYELTVQIENMKDTATVCIRVVEKAGPP
QNVMVKEVWGTNALVEWQPPKDDGNSEITGYFVQKADKKTMEWFTVYERNRHTSCTVSDLIMGNEYYFRVYSENVCGLSDLPGVSKNTARIVK
TGMTLKLPEYKEHDFRTPPKFLTPLPDRVVVAGYAAALNCAVRGHPKPKVVWMKNKMEIREDPKFLMTNQQGVLTLNIRRPSPFDSGTYSCRA
VNELGEALAECKLEVRAPR (SEQ ID NO: 36)
>sp|Q05623|MYBPH_CHICK Myosin-binding protein H OS = Gallus gallus OX = 9031 GN = MYBPH
PE = 1 SV = 1
MTGKTAPAAAKKAPAAKKAPAPASKKAPEPAPKEKPAPTPKEGHAPTPKEEHAPPPKEEHAPPPKEEHAPAPAAETPPAPEHPPDAEQPAAPA
AEHAPTPTHEAAPAHEEGPPPAAPAEAPAPEPEPEKPKEEPPSVPLSLAVEEVTENSVTLTWKAPEHTGKSSLDGYVVEICKDGSTDWTAVNK
EPFLSTRYKIHDLASGEKVHVRVKAISASGTSDPATLEQPVLIREITDLPRIRLPRQLRQVYVRHVGEAVNLLIPFQGKPQPQVTWTKDNQPL
DTSRVNIRNTDKDTIFFIRTAQRSDSGKYQLSVRINGAEDKAILDIRVIERPGPPQNLKLVDVWGFNVALEWSPPADNGNSEIKGYTVQKSDK
KSGKWFTVLERCTRTSCTISDLIIGNTYSFRVFSENACGMSETAAVAAGVAHIKKTVYQPQKIPERDMMEPPKFTQPLTDRATTRGYSTHLFC
SVRGFPQPKIIWMKNKMEIREDPKYIAMIEQGVCSLEIRKPSPFDAGVYTCKAVNPLGEASVDCKLDVKMPK (SEQ ID NO: 37)
>tr|G3X6W9|G3X6W9_BOVIN Myosin binding protein H OS = Bos taurus OX = 9913 GN = MYBPH PE = 4
SV = 1
MTEKATSEAPACGLEETTSESAHVPLTEPSGDTAAPQAPGGEQAPRGQQASDPQESARQPPDPAASAAPAGPAATDPALPREDVP
SAPLLLAVEDVSDSSVTVSWEPPERLGRLGLQGYVLELRREGALDWVPVNARPMMVTQQTLRNLAVGDKFFVRVAAVSSAGAGPPAVLERLIH
IQETIEAPKIRVPRHLRQTYIRQVGESINLQIPFQGNPKPQALWTHNGHALDSQRVSVRTGDQDSILFIRSAQRSDSGCYELTVQLKDLEAKA
AINILVIEKPGPPRSIRLLDVWNCNATLEWTPPQDTGNTELLGYTVQKADKKTGQWFTVLERCHPTSCTVSDLIVGNSYSFRVFSENLCGLSA
SAAVTKELAHIVKTDIVAKPKSFVERDFSEAPSFTQPLADHTSTPGYSTQLSCSVRASPKPKIIWMKNKMDIQGDPKYRALSEQGVCTLEIRK
PSPFDSGVYTCKAINVLGEASVDCRLEVKASATH (SEQ ID NO: 38)
>tr|A6BM71|A6BM71_CHICK Connectin (Fragment) OS = Gallus gallus OX = 9031 GN = TTN PE = 2
SV = 1
MTTKAPTFTQPLQSVVALEGSAATFEAHISGFPVPEVSWYRDGQVLSAATLPGVQISFSDGRAKLVIPSVTEANSGRYTIQATNGSGQATSTA
ELLVTAGTAPPNFSQRLQSMTARQGSQVRLDVRVTGIPTPVVKFYRDGVEIQSSPDFQILQEGDLYSLIIAEAYPEDSGTYSVNATNNVGRAT
STAELLIQGEEEAVPAKKTKTIVSTAQISQTRQARIEKKIETHFDARSLTSVEMVIEGAAAQQLPHKAPPRMPPRPTSKSPTPPVITAKAQMA
RQQSPSPVRQSPSPVRHVRAPTPSPVRSVSPAGRISTSPIRPVKSPSPIRKAQVVTPGAEVLPPWRQEGYSATAEAQMKETRVSTSATEIRTE
ERWEGRYGLQEQVTISGAAAGEVAAGAKEVRKEPEKTPVPTVIIATDKAKEQERISTAREEISARHEQVHVSHEQIEAGKRAEAVATVVAAVD
QARVRSPWETEQVDETYVKKKTLEYGYKEHAVKDHEAQAEHHVATKEVKTVYVPPEKHIPAAEKKEVHVSTEIKRETEAKIEKTIHIEHPRPR
TASPHFTVSKIAVPKPDHTYEVSIAGSAMATLEKELSATSAAQKITKPVKPPQLKPHEVKIKPESAPPQFPFTEAAETYKAHYDVETKKEVDV
SIKGEAVREDHLLLRKESEAKVTETARVPVPAEIPVTPPTLVWGLKNKTVTEGESVTLECHISGHPQPTVTWYREDYKIESSMDFQITFKAGL
ARLVIREAFAEDSGRFTCTATNKAGSVSTSCHLHVKVSEETETRETISEKVVTEEKSYVETKDVVMEDVSAAAEEVSGEPVPPFFIRKPVVHK
LIEGGSIIFECQVGGNPKPHVLWKKGGVPLTTGYRYKVSYKRETGECKLEISMTFADDAGEYTIVIRNKFGEASATVSLLEEADYEAYIKSQQ
EMMYQTQVTAYVQEPKVAEVAPPISYGDFDKEYEKEQALIRKKMAKDTVMVRTFVEDEEFHISSFEERLIKEIELRIIKTTLDELLEEDGEEM
MIDISESEAIGAGFDLRLKNYRTFEGTGVTFHCKTTGYPLPKIAWYKDGKRIRHGERYHMEVLQDGSASLRLPVVLPEDEGIYTVFASNMKGN
AICSAKLYVEPVAPTATPGYMPGPEVMRRYRSISPRSPSRSPARSSPSCSPARRLDETDEGQLERLYKPVFVLKPTSVKCSQGQTARFDLKVV
GRPMPETYWFHNGQQVVNDYTHKIVIKEDGTQSLIIVPAMPEDSGEWAVIAQNRAGKASVSVTLSVEAKEDLVRPRFVERLRNVSVKEGSRLH
MAVKATGNPNPDIVWLKNSDIIVPHKYPRIRIEGTKGAAALNIESTARQDAAWYTATAINKAGRDTTRCKVNVEVEHAEPEPERRLIIPKGTY
KAKEIAAPELEPLHLRYGQEQWEEGDLYDKEKQQKPFFKKKLTSLRLKQFGPAHFECRLTPIGDPTMVVEWLHDGKPLEAANRLRMINEFGYC
SLDYGVAYSRDSGVITCRATNKYGTDHTSATLIVKDEKSLVEESQLPEGRRGMQRIEELERMAHEGALPAVAVDQKEKQKPELVLVPEPARVL
EGETARFRCRVTGYPLPKVNWYLNSQLIRKSKRFRLRYDGIHYLDIVDCKSYDTGEVKVTAENPEGFIEHKVKLEIQQREDFRSVLRRAPEPR
HEPVVTEPGKLLFEVQKIDKPAEATTKEVVKLKRAERITHEKLSEESEELRSKFKRRTEEGYYEAITAVELKSRKKDESYEEMLKKTKEELLH
WTKEIPEEEKKALPPEGKITIPTFKPEKVELSPSMEAPKIFERIQSQTVAQGTDAHFRVRVVGKPDPECQWFRNGVQIERTDRIYWYWPEDNV
CELVIRDVTADDSASIMVKAVNIAGETSSHAFLLVQAKQLISFIQNLQDVVAKERDSMATFECETSEPFIKVKWFKNGIEIHSGEKYRMHSDR
KAHFLSVLAVEMSDADDYSCALVEDESVKTTAKLIVEGAVVEFIKELEDVEVPESFTGELECEVSPEDIEGKWYHGDVELSSNHKYVLASRRG
RRILTIKDVNKDDQGEYSFVVDGKRTHCKLKMKPRPMTILQGLTDQKVCEGDIVQLEVKVSVENVEGVWMKDGHEIQSSDRIHIVLDKQSHML
LIEDATQEDSGTYSFSIPGLELSTTGQVTVYSVEIIVPLKDVHVVEGTKAILECKVSAPDVTSSKWYLNDHQIKPDERVQAVCKGTKQRLVIT
RTHASDEGHYKLMVGKVETSCNVTVEEIEIIRGLHDITCTETQNVSFEVELSHSGIDVIWHFKGQEIKAGPKYKIEARGKIYKLTVVKMMKDD
EGEYVFYAGGKKTSGKLIVAGGAISKPLADLTVAESQRAVFECEVANPESEGQWLKNGKPLPMTDQYRAETDGVKRRLNIPAAKMDDMGEYSY
EIASSKTSAKLHVEAVKIKKTLKNLTVTETQEAVFSVELSHPDVKGALWIKNGVELESNDKYEISVKGTVHTLKIKHCVVTDESVYSFKLGKI
GANARLHVETVKIIKKPKDVTALENAVVSFELSVSHDTVPVRWFHKNVELKQSDKYKMISQRKVHKLMLHNISPADAGEYTAFVGQLECKAKL
FVETIHITKTMKSIEIPETKTASFQCEVSHFNVPSVWLKNGVEIEMSEKFKIVVQGKLHQLNIMNTSSEDSAEYTFVCGNDRVSATLTVKPIL
ITSMLEDINAEEKDTITFEVTVNYEGISYKWLKNGVEIKSTDKCQIRTKKLTHSLSIRNVHFGDAAEYSFVAGKAASSATLYVEARHIEFRKH
IKDIKVVEKKRAIFECEISEPDVQVQWMKDGQELQIGDRMKIQREKYVHRLIIPSTKMSDAGQYTVVAGGNTSSANLIVEGRDVRIRSIRKEI
QVIERQRAEIEFEVNEDDIEPQWYKDGIEINFHYEERYSYVVERRIHRMSIFETTYSDAGEYTFVAGRNRSSVVLYVNAPEPPQIIQELQPTT
VESGKPARFCAIISGKPQPKVSWYKDDQQLSPGFKCKFLHDAQEYTLLLIETFPEDSAVYTCEAKNDYGVATTSASLSVEIPEVVSPELEVPV
YPPAVIVPLRDAVTSEGQSARFQCRVTGTDLKVSWYSKDREIKPSRFFRMTQFEDTYQLEIAEAYPEDEGTYTFVASNSVGQVTSTAILKLEA
PEKIMYEKLEEEIEMEVKVAPILRRRLEPLEVAVNHVAKFTCEVETTPNVKFQWYKAGREIYDGDKYSIRSSNYLSTLEIPRPQVVDCGEYSC
KASNQHGSVSSTAFLTVTEPPRFIKKLDSSRLVKQHDSTRYECKVGGSPEIKVTWYKGETEIHPSEKYSMSFVDSVAVLEMHNLSVEDSGDYS
CEAQNPAGSASTSTSLKVKAPPAFTKKPHPVQTLKGSDVHLECELQGTPPFQISWYKDKREIRSSKKYKVMSENYLASIHILNVDTADVGEYH
CKAVNDVGSDSCIGSVTLRAPPTFVKKLSDVTVVVGETIELQAAVEGAQPISVLWLKDKGEIIRESENLWISYSENVASLKIGNAEPTNAGKY
ICQIKNDAGFQECFAKLTVLEPAVIVEKPGPVKVTAGDSCTLECTVDGTPELTARWFKDGNELSTDHKYKISFFNKVSGLKILNAGLEDSGEY
TFEVKNSVGKSSCTASLQVSDRIMPPSFTRKLKETYGQLGSSAVLECKVYGSPPILVSWFHDGQEITSGDKYQATLTDNTCSLKVNGLQESDM
GTYSCTATNVAGSDECSAFLSVREPPSFVKKPEPFNVLSGENITFTSIVKGSPPLEVKWFRGSIELAPGHKCNITLQDSVAELELFDVQPLQS
GDYTCQVSNEAGKISCTTHLFVKEPAKFVMKVNDLSVEKGKNLILECTYTGTPPISVTWKKNGVILKHSEKCSITTTETSAILEIPNSKLEDQ
GQYSCHIENDSGQDNCHGAITILEPPYFVTPLEPVQVTVGDSASLQCQVAGTPEMIVSWYKGDTKLRGTATVKMHFKNQVATLVFSQVDSDDS
GEYICKVENTVGEATSSSLLTVQERKLPPSFTRKLRDVHETVGLPVTFDCGIAGSEPIEVSWFKDNVRVKEDYNVHTSFIDNVAILQILKTDK
SLMGQYTCTASNAIGTASSSGKLVLTEGKTPPFFDTPITPVDGIIGESADFECHISGTQPIRVTWAKDNQEIRTGGNYQISYVENTAHLTILR
VDRGDSGKYTCYASNEVGKDSCTAQLNVKERKTPPTFTRKLSEAVEETEGNELKLEGRVAGSQPLTVSWYKNNQEVHSSPHCEISFKNNTLLL
HIKSVGQSDAGLYTCKVSNEAGSVLCTSSVVIREPKKPPVFDQPLQPAATEEGDTLQLSCHVRGSEPIRIQWLKAGREIRASERCSFSFANGV
ALLELAAVTKSDSGEYVCKASNVAGTDTCRSKVTVKEKAALVSAAKKADIEGKLYFVSEPQSIKVMEKTVATFIAKVGGDPIPNVKWMKGKWR
QLNQGGRIIIQQRGDEAKLEIKDTTKTDSGLYKCVAFNQHGEIERSVNLQVEERKQEVVEEDVRGKLKRIPTKKKEDEEQTIDILELLKNVDP
KEYEKYARMYGITDFRGLLQAFELLKQTREEESHRLEIELTEKAQKEDQEFEELVAFIQQRLTQTEPVTLIRDIENQTVLTDEDAIFECEIKI
NYPEIKLSWYKGTQKLDSSDKYKIKIEGDRHILKIKNCQLEDQGNYRIVCGPHIASARLTVIEPAVERHLHDTTFKEGNTCTLSCQFSIPNAK
SQWYRNGRPIKIGGRYSTQVSDKVHKLIIKDVRTEDQGQYTCKLDNLETTADLTIEAEPIQFTKSIQNIVVSEHQSATFECEVSFDDAVVTWY
KGPTELTESPKYSFRSEGRCHYMTIHNVTAEDEGVYSVIARLEPRGEARSTAELYLVTKEIKLELKPPDVPDAKVAVPPQKPAEAAPIPILLP
LIPTPEEKKPAEKKVPVKKVSKKVVKKGPKEIPPPEVPEILPEKPKEVKITSMARREEIHEEKMEIYEKPKKVYEEWEEDYGEDHDYYFKEEG
YDEGEEEWEETYDKREVAYEEEQIIHEEVVEVPKKPVPERKPPAITAEKKEKKEVTVHKVPDVPKKIPEEKVPITVPKKPEPTPAKEPEVHKI
IEEKIKVSEPKKPEVPPAKVPEVPKKVVKEEVSVEVPKKPEPPPAKVPEVPKKVVPEEKVHVEVPKKAEPPPPKVLRKKPEEEEPKPEKEPKP
EVKPVPTPVEKIKKPEVPEVPKKKTEIPVIKKEEKHVPEPPKEPKPAAISVPGPEPKPAVALKSPKPAAEPAPAITTAPVTTPVVGKKAEAKP
TKEETPKGISPVKAKKTPSPADAERRKLRPGSGGEKPPEEPPFTYQLKAVPLKFVKEMKDIVLKEAESVGSSAIFEVLISPSTAITSWMKDGS
NIRESPKHKFIADGKDRKLHIIDVQLSDAGEYTCVLRLGNKEKTSTAKLIVEELPVRFVKTLEEEVTVVKGQPLYLTCELNKERSVVWRRDGK
IIKDKPGKFALGIIGLSHSLTITDSDDSDAGTYTVTVEDSELSCSSCVKVVEVIRDWLVKPIRDQHVKPKGTATFTCDIVKDTPNIKWFKGDE
EIPAEPTDKTEILKEGNKIFLKIKNAGPADIGEYSVEVEGRRYPAKLTLGEREVELLKPLEDVTVYEKETANFDTEISEEDIPGEWKLKGEIL
RPSPTCEIKAEGGKRFLTLHKVKLEQAGEVLYQALNAVTTAILTVKEIELDFAVPLKDVTVPEKRQARFECVLTREANVIWSKGTDILKIGEK
FDIIADGKKHILVINDSQFDDEGEYTAEVEGKKSTARLFVEGVRLKFITPLQDQTVKEGETAYFQFELSHEGMLVKWYKNDKRLHTSRTVFIT
SEGKVHKLEMREVTLDDISEIKAVVKDMNTQANLKVLEADPYFTVKLQDYSALEKDDITLQCELSKDVPVKWYKDGEELVASSRISIKTDGLR
RILTIKKATEGDKGVYECSCGTDKINCNIGVEPRLIKVERPLYGVEVFVGETARFEIEISEPDVHPIWKLKGETLTPSPECEIIEDGKKHILI
LHQGRLDMTGEVSFQAANAKSAANLKVKELPLIFITPLSDVKVFEKDEAKFECEVSREPKTFRWLKGTQEITPDERFEIISDGTKHALIIKSV
AFDDEAKYMFEAEDKRTSAKLIIEGIRLKFITPLKDVTKKERETAVFTVELSHENIPVVWFKNDQRLHTSKVVSMTDDGKFHTLTIKDLTIDD
TSQIRVEAMGKSSEAKLTVLEGDPYFTGKLQDYTAVEKDEVILQCEISKADAPVKWMKDGKPITPSKNVVIKADGKKRILILKKALKKDIGQY
TCDCGTDQTSANLNIEDRDIEIIRPLYSVEVIETETARFDIEISEEGVHGNWKLKGEPLTESPDCEIKEEGKKHFLTLYNVRLDQAGGVDFQA
ANAKSGAHLRVKPRVIGLLRPLKDVTVTAGESATFDCELSYEGIPVEWYLQGKKLEPSDKVVTRAEGRAHTLILRDVKLTDAGEVSLTAKDER
TQANLFVKEPPVEFTKPLEDQTVEEEATAILECEVSRENAKVKWFKNGEEIHKTKKYDIISEGRVRKLIIHGCTLDDAKTYTCDAKDFKTSCF
LNVEPLRVEFLRPLTDLEVKEKRICSVLNVKFLAQGVKVKWFKDGIEIEKAEYDIISKGAERILVISKCLEDDEAEYACEAKTARTSGLLTVI
EEEAVFTKNLHDLEVNENETVRLICEISKPNAEVTWFKGDEEVPDGGRFEYISDGRKRILVIRNAHPEDAGKYTCKLPSSSTTGKLTVHELAA
EFLTRPQNLEVLEGDKAEFACSVSKEAITVQWLWGDTVLEPGDKYDIISDGKKRTLVVKDSVVGDAGKYTVMVGEAKATARLTVIEKTQDYNS
LKDQEVNEGQEIIFNCEVNKEGAKEKWYKNGEAIFDSAKYIIVQKDLVYTLRIRDTQLKDQATYSISLSNHRGEHAESSAALTVLEEGLRIVE
PLEDIETMEKKTVTFWCKVNRLNATLKWTKNGEEITENKRILYKIDKYKHSLIIKDCGFKDEGEYTVTAGQDKSVAELLITEAPADFIEHLQD
QTVTEFDDAVFTCQLSKEKASVKWYRNGREIKEGKKYQFEKDGNLHRLIIKDCRLDDECEYSCGVDDRKSRARLFVEEIPVEIIRPPQDVYEA
PGADVIFMAELNKDGVEVKWLRNNMIIIQGDKHQMMSEGKVHRLQVCEIKPRDQGEYRFIAKDKEARAKLELAAAPRIKTGDQNLVVDVGNPL
TMTVPYDAYPRAEAEWLKGEESLPTTTVDTTTDCTTFKIYEAKKSDKGRYKVVLRNKHAQAEAFINVEVIDVPGPVRNLEVTEIYDGEVGLAW
QEPESDGGSKIIGYVVERRDIKRKTWIVVTDHAENCEYTVTGLQKGGVEYLFRVSARNRVGTGEPVETERPVEAKSKFEVPGPPQNVEVTDVN
RFGRTLTWEAPEYDGGSPITGYVIELRNRASIKWEPTMTTGADELSAVLTDVVENEEYFFRVRAQNMVGVGKPSHPTRAVKITDPIERPSGNI
NLDHSDQTKTSVQLTWEPPLEDGGSPILGYIIERKEEGTDKWIRCNPKLVPACAFKVTGLKAGSSYYYRVSAENAAGVSDPAEAIGPLTADDP
FVDLQWPLSAFKDGLEVIVPEPIKIRVPITGYPIPTATWSFGDQVLEEGGRVTMKTTSTFAELVITPSERPDKGIYTLTLENPVSSVSGEIDV
NVLARPSAPKELKVVDVSRSTVQLSWEPPEDDGGSPVIDYIIEKREVSRKTWIKVMDHVIDQEFSVPDLIQGKEYLFRVCACNKCGPGEPAYI
DEPINMSAPATVPDPPENVKWRNPTSKGIFLTWEPPKYDGGARIKGYLVEKCQRGTDKWEICGEPVIETKMEVTKLKEGEWYAYRVKALNRIG
ASKPSKPTDDIQAIDAKEAPEIFLDVKLLAGLTVKAGTKIELPAKITGKPEPQITWTKAEKLLRPDDRITIETTPNHSTVTITDSKRSDSGTY
IIEAVNSSGRATAVVEVNVLDKPGPPAAFDVSEITNESCLLTWNPPRDDGGSKITNYVLEKRATDSEIWHKLSSTI (SEQ ID NO: 39)
>A6BLM7|A6BLM7_CHICK Titin isoform Ch12 (Fragment)
EGEEEWEETYDKREVAYEEEQIIHEEVVEVPKKPVPERKPPAITAEKKEKKEVTVHKVVKKPVDEKVEITTQRVAEEKLKQAEVT
KKIPSAKPTPMIIEEKVMKKEAHKEVEEEEEREVISEELETHHVEVPDVPKKIPEEKVPITVPKKPEPTPAKEPEVHKIIEEKIKVSEPKKPE
VPPAKVPEVPKKVVKEEVSVEVPKKPEPPPAKVPEVPKKVVPEEKVHVEVPKKAEPPPPKVLRKKPEEEEPKPEKEPKPEVKPVPTPVEKIKK
PEVPEVPKKKTEIPVIKKEEKHVPEPPKEPKPAAISVPGPEPKPAVALKSPKPAAEPAPAITTAPVTTPVVGKKAEAKPTKEETPKGISPVKA
KKTPSPADAERRKLRPGSGGEKPPEEPPFTYQLKAVPLKFVKEMKDIVLKEAESVGSSAIFEVLISPSTAITSWMKDGSNIRESPKHKFIADG
KDRKLHIIDVQLSDAGEYTCVLRLGNKEKTSTAKLIVEELPVRFVKTLEEEVTVVKGQPLYLTCELNKERSVVWRRDGKIIK (SEQ ID
NO: 40)
>A6BLM8|A6BLM8_CHICK Titin isoform b11 (Fragment)
SDKYKIKIEGDRHILKIKNCQLEDQGNYRIVCGPHIASARLTVIAEPIQFTKSIQNIVVSEHQSATFECEVSFDDAVVTWYKGPT
ELTESPKYSFRSEGRCHYMTIHNVTAEDEGVYSVIARLEPRGEARSTAELYLVTKEIKLELKPPDVPDAKVAVPPQKPAEAAPIPILLPLIPT
PEEKKPAEKKVPVKKVSKKVVKKGPKEIPPPEVPEILPEKPKEVKITSMARREEIHEEKMEIYEKPKKVYEEWEEDYGEDHDYYFKEEGYDEG
EEEWEETYDKREVAYEEEQIIHEEVVEVPKKPVPERKPPAITAEKKEKKEVTVHKVPDVPKKIPEEKVPITVPKKPEPTPAKEPEVHKIIEEK
IKVSEPKKPEVPPAKVPEVPKKVVKEEVSVEVPKKPEPPPAKVPEVPKKVVPEEKVHVEVPKKAEPPPPKVLRKKPEEEEPKPEKEPKPEVKP
VPTPVEKIKKPEVPEVPKKKTEIPVIKKEEKHVPEPPKEPKPAAISVPGPEPKPAVALKSPKPAAEPAPAITTAPVTTPVVGKKAEAKPTKEE
TPKGISPVKAKKTPSPADAERRKLRPGSGGEKPPEEPPFTYQLKAVPLKFVKEMKDIVLKEAESVGSSAIFEVLISPSTAITSWMKDGSNIRE
SPKHKFIADGKDRKLHIIDVQLSDAGEYTCVLRLGNKEKTSTAKLIVEELPVRFVKTLEEEVTVVKGQPLYLTCELNKERSVVWRRDGKIIKD
KPGKFALGIIGLSHSLTITDSDDSDAGTYTVTVEDSELSCSSCVKVVEVIRDWLVKPIRDQHVKPKGTATFTCDIVKDTPNIKWFKGDEEIPA
EPTDKTEILKEGNKIFLKIKNAGPADIGEYSVEVEGRRYPAKLTLGEREVELLKPLEDVTVYEKETANFDTEISEEDIPGEWKLKGEILRPSP
TCEIKAEGGKRFLTLHKVKLEQAGEVLYQALNAVTTAILTVKEIELDFAVPLKDVTVPEKRQARFEC (SEQ ID NO: 41)
>P79757|P79757_CHICK Connectin/titin (Fragment)
KSRLRRRREREITEITSEEEEEIEMVQHVHREFSPPSRLLRRRRSLSPTYIELMRPVSELIRPRSRPPEESERRSPTPERTRPRS
PSPVSTERSLSRFERMARFDIFSRYESMKSALKTQKTMERKYEVLTQQPFTLDHAPRITLRMRSHRVPCGHNTRFILNVQSKPTADVKWYHNG
IELQESSKIHFSNTSGVLTLEILDCHIDDSGTYRAVCTNYKGECSDYATLDVTGGDYTTYSSQRRDEEVPRSILPDLTRTEAYAVSSFKKATA
AEASSSVREVKSEVSATRESLLSYEHHASSEEKITASEEKSLEERTVHKAFKSTLPATILTKPRSITVSEGETARFSCDVDGEPAPTITWVRA
GQPIVSSRRFQITRTQYKSTFEISLVQIADEGSYTVVVENSEGRQEAHFTLTVQRKRIPEKAITSPPRIKSPEPRVKSPEPVKSPKRVKSPEP
ISTPSKAKSPPGDKTAPVEKVQLPTASPPKIKEQLKAETLGDKVKLSCAVESSVLSIREVAWYKDGKKLKEDHHFKFHYAADGTYELKIHNLT
ESDKGEYTCEIMGEGGISKTNFQFTGQVFKNIHSQVQSVSETPKSVEKGDKVLAVSTQKKSSAATEEKAAIEEVIKKSIVTEDVKQLQAEIRA
SSTQMTVSEGQKVTLKANIPGASEVKWVLNGMELRNSDDYRYGISGSNHTLTIKKASNKDEGILTCEGKTDEGTIKCQYVLTFSKEPSNEPAF
ITQPKSQNVNEGQDVLFTCEVSGDPSPEVEWLRNNQPIAVSSHMRATRSKNTYSLEIRNAAVSDTGKYTVKAKNYHGQCSATASLTVFPLIEE
PPKEVVLKTSGDASMHESFSSQSFQMAASKQEASFSSFSSSSMTETKFASMSAKSMSSMKESFVEMSSSSIMGKSSMAQLESSTSKMLKSGVR
GVPPKIEALPSDISIDEGKVLTLSCAFSGEPAPEITWYCRGRKITSQDQQGRFHIETSEDLTTLIIMDVQKNDGGIYTLNLGNEFGTDSATVN
INIRS (SEQ ID NO: 42)
>BAA11908.1 connectin/titin, partial [Gallus gallus]
MTTKAPTFTQPLQSVVALEGSAATFEAHISGFPVPEVSWYRDGQVLSAATLPGVQISFSDGRAKLVIPSVTEANSGRYTIQATNGSGQATSTA
ELLVTAGTAPPNFSQRLQSMTARQGSQVRLDVRVTGIPTPVVKFYRDGVEIQSSPDFQILQEGDLYSLIIAEAYPEDSGTYSVNATNNVGRAT
STAELLIQGEEEAVPAKKTKTIVSTAQISQTRQARIEKKIETHFDARSLTSVEMVIEGAAAQQLPHKAPPRMPPRPTSKSPTPPVITAKAQMA
RQQSPSPVRQSPSPVRHVRAPTPSPVRSVSPAGRISTSPIRPVKSPSPIRKAQVVTPGAEVLPPWRQEGYSATAEAQMKETRVSTSATEIRTE
ERWEGRYGLQEQVTISGAAAGEVAAGAKEVRKEPEKTPVPTVIIATDKAKEQERISTAREEISARHEQVHVSHEQIEAGKRAEAVATVVAAVD
QARVRSPWETEQVDETYVKKKTLEYGYKEHAVKDHEAQAEHHVATKEVKTVYVPPEKHIPAAEKKEVHVSTEIKRETEAKIEKTIHIEHPRPR
TASPHFTVSKIAVPKPDHTYEVSIAGSAMATLEKELSATSAAQKITKPVKPPQLKPHEVKIKPESAPPQFPFTEAAETYKAHYDVETKKEVDV
SIKGEAVREDHLLLRKESEAKVTETARVPVPAEIPVTPPTLVWGLKNKTVTEGESVTLECHISGHPQPTVTWYREDYKIESSMDFQITFKAGL
ARLVIREAFAEDSGRFTCTATNKAGSVSTSCHLHVKVSEETETRETISEKVVTEEKSYVETKDVVMEDVSAAAEEVSGEPVPPFFIRKPVVHK
LIEGGSIIFECQVGGNPKPHVLWKKGGVPLTTGYRYKVSYKRETGECKLEISMTFADDAGEYTIVIRNKFGEASATVSLLEEADYEAYIKSQQ
EMMYQTQVTAYVQEPKVAEVAPPISYGDFDKEYEKEQALIRKKMAKDTVMVRTFVEDEEFHISSFEERLIKEIELRIIKTTLDELLEEDGEEM
MIDISESEAIGAGFDLRLKNYRTFEGTGVTFHCKTTGYPLPKIAWYKDGKRIRHGERYHMEVLQDGSASLRLPVVLPEDEGIYTVFASNMKGN
AICSAKLYVEPVAPTATPGYMPGPEVMRRYRSISPRSPSRSPARSSPSCSPARRLDETDEGQLERLYKPVFVLKPTSVKCSQGQTARFDLKVV
GRPMPETYWFHNGQQVVNDYTHKIVIKEDGTQSLIIVPAMPEDSGEWAVIAQNRAGKASVSVTLSVEAKEDLVRPREVERLRNVSVKEGSRLH
MAVKATGNPNPDIVWLKNSDIIVPHKYPRIRIEGTKGAAALNIESTARQDAAWYTATAINKAGRDTTRCKVNVEVEHAEPEPERRLIIPKGTY
KAKEIAAPELEPLHLRYGQEQWEEGDLYDKEKQQKPFFKKKLTSLRLKQFGPAHFECRLTPIGDPTMVVEWLHDGKPLEAANRLRMINEFGYC
SLDYGVAYSRDSGVITCRATNKYGTDHTSATLIVKDEKSLVEESQLPEGRRGMQRIEELERMAHEGALPAVAVDQKEKQKPELVLVPEPARVL
EGETARFRCRVTGYPLPKVNWYLNSQLIRKSKRFRLRYDGIHYLDIVDCKSYDTGEVKVTAENPEGFIEHKVKLEIQQREDFRSVLRRAPEPR
HEPVVTEPGKLLFEVQKIDKPAEATTKEVVKLKRAERITHEKLSEESEELRSKFKRRTEEGYYEAITAVELKSRKKDESYEEMLKKTKEELLH
WTKEIPEEEKKALPPEGKITIPTFKPEKVELSPSMEAPKIFERIQSQTVAQGTDAHFRVRVVGKPDPECQWFRNGVQIERTDRIYWYWPEDNV
CELVIRDVTADDSASIMVKAVNIAGETSSHAFLLVQAKQLISFIQNLQDVVAKERDSMATFECETSEPFIKVKWFKNGIEIHSGEKYRMHSDR
KAHFLSVLAVEMSDADDYSCALVEDESVKTTAKLIVEGAVVEFIKELEDVEVPESFTGELECEVSPEDIEGKWYHGDVELSSNHKYVLASRRG
RRILTIKDVNKDDQGEYSFVVDGKRTHCKLKMKPRPMTILQGLTDQKVCEGDIVQLEVKVSVENVEGVWMKDGHEIQSSDRIHIVLDKQSHML
LIEDATQEDSGTYSFSIPGLELSTTGQVTVYSVEIIVPLKDVHVVEGTKAILECKVSAPDVTSSKWYLNDHQIKPDERVQAVCKGTKQRLVIT
RTHASDEGHYKLMVGKVETSCNVTVEEIEIIRGLHDITCTETQNVSFEVELSHSGIDVIWHFKGQEIKAGPKYKIEARGKIYKLTVVKMMKDD
EGEYVFYAGGKKTSGKLIVAGGAISKPLADLTVAESQRAVFECEVANPESEGQWLKNGKPLPMTDQYRAETDGVKRRLNIPAAKMDDMGEYSY
EIASSKTSAKLHVEAVKIKKTLKNLTVTETQEAVFSVELSHPDVKGALWIKNGVELESNDKYEISVKGTVHTLKIKHCVVTDESVYSFKLGKI
GANARLHVETVKIIKKPKDVTALENAVVSFELSVSHDTVPVRWFHKNVELKQSDKYKMISQRKVHKLMLHNISPADAGEYTAFVGQLECKAKL
FVETIHITKTMKSIEIPETKTASFQCEVSHFNVPSVWLKNGVEIEMSEKFKIVVQGKLHQLNIMNTSSEDSAEYTFVCGNDRVSATLTVKPIL
ITSMLEDINAEEKDTITFEVTVNYEGISYKWLKNGVEIKSTDKCQIRTKKLTHSLSIRNVHFGDAAEYSFVAGKAASSATLYVEARHIEFRKH
IKDIKVVEKKRAIFECEISEPDVQVQWMKDGQELQIGDRMKIQREKYVHRLIIPSTKMSDAGQYTVVAGGNTSSANLIVEGRDVRIRSIRKEI
QVIERQRAEIEFEVNEDDIEPQWYKDGIEINFHYEERYSYVVERRIHRMSIFETTYSDAGEYTFVAGRNRSSVVLYVNAPEPPQIIQELQPTT
VESGKPARFCAIISGKPQPKVSWYKDDQQLSPGFKCKFLHDAQEYTLLLIETFPEDSAVYTCEAKNDYGVATTSASLSVEIPEVVSPELEVPV
YPPAVIVPLRDAVTSEGQSARFQCRVTGTDLKVSWYSKDREIKPSRFFRMTQFEDTYQLEIAEAYPEDEGTYTFVASNSVGQVTSTAILKLEA
PEKIMYEKLEEEIEMEVKVAPILRRRLEPLEVAVNHVAKFTCEVETTPNVKFQWYKAGREIYDGDKYSIRSSNYLSTLEIPRPQVVDCGEYSC
KASNQHGSVSSTAFLTVTEPPRFIKKLDSSRLVKQHDSTRYECKVGGSPEIKVTWYKGETEIHPSEKYSMSFVDSVAVLEMHNLSVEDSGDYS
CEAQNPAGSASTSTSLKVKAPPAFTKKPHPVQTLKGSDVHLECELQGTPPFQISWYKDKREIRSSKKYKVMSENYLASIHILNVDTADVGEYH
CKAVNDVGSDSCIGSVTLRAPPTFVKKLSDVTVVVGETIELQAAVEGAQPISVLWLKDKGEIIRESENLWISYSENVASLKIGNAEPTNAGKY
ICQIKNDAGFQECFAKLTVLEPAVIVEKPGPVKVTAGDSCTLECTVDGTPELTARWFKDGNELSTDHKYKISFFNKVSGLKILNAGLEDSGEY
TFEVKNSVGKSSCTASLQVSDRIMPPSFTRKLKETYGQLGSSAVLECKVYGSPPILVSWFHDGQEITSGDKYQATLTDNTCSLKVNGLQESDM
GTYSCTATNVAGSDECSAFLSVREPPSFVKKPEPFNVLSGENITFTSIVKGSPPLEVKWFRGSIELAPGHKCNITLQDSVAELELFDVQPLQS
GDYTCQVSNEAGKISCTTHLFVKEPAKFVMKVNDLSVEKGKNLILECTYTGTPPISVTWKKNGVILKHSEKCSITTTETSAILEIPNSKLEDQ
GQYSCHIENDSGQDNCHGAITILEPPYFVTPLEPVQVTVGDSASLQCQVAGTPEMIVSWYKGDTKLRGTATVKMHFKNQVATLVFSQVDSDDS
GEYICKVENTVGEATSSSLLTVQERKLPPSFTRKLRDVHETVGLPVTFDCGIAGSEPIEVSWFKDNVRVKEDYNVHTSFIDNVAILQILKTDK
SLMGQYTCTASNAIGTASSSGKLVLTEGKTPPFFDTPITPVDGIIGESADFECHISGTQPIRVTWAKDNQ (SEQ ID NO: 43)
>tr|Q07784|Q07784_CHICK Titin (Fragment) OS = Gallus gallus OX = 9031 PE = 2 SV = 1
RESDHRAWTPVSYTVTRQNAVVQGLTEGKAYFFRIAAENIIGMGPFTETTKELIIRDPITVPDRPEDLEVKAVTKNSVTLTWNPPKYNGGSDI
TMYVLESRLIGKEKFHRVTKEKMLDRKYTVEGLKEGDTYEYRVSACNIVGQGKPSFCTKPITCKDEIAPPTLDLDFRDKLIVRVGEAFSLTGR
YSGKPTPKITWLRDDIVLKEDDRTKIKTTPTTLCLGILKSVREDSGKYCVTVENSTGSRKGFCQVTVVDRPSPPVGPVIFDEVHKDHMIVSWK
PPLDDGGSEITNYIIEKKDTHRDLWMPVTSATVKTTCKIPKLLEGREYQVRIYAENLYGISDPLISDEMKAKDRFSVPDAPEQPVIKDVTKDS
AVVVWNKPYDGGKPITNYIVEKKETMATRWVRVTKDPIFPSTQFKVPDLLEGCQYEFRVSAENEIGVGNPSPPSKPIFARDPIVKPSPPVNPE
AVDKTKNSVDLTWQPPRHDGNGKIIGYLVEYQKVGDEEWKKANLTPDSCPETKYKVTGLTEGLTYKFRVMAVNAAGESEPAYVPDPVEVKDRL
EPPELILDANMAREQHVRAGDTLRLSAVIKGVPFPKVSWKKEDKEVSPKADIEVTGVGSKLEIRNTVHEDGGIYSLTVENPAGSKTVSVKVLV
LDKPGPPRNLQVSDVRSDSCYLTWKEPEDDGGSVITNYVVERKDVASAQWVSVSSSSKKRSHMAKYLMEGTQYLFRVAAENLFGRGPYVETLK
PIKAMDPLHPPGPPKNLHHIDVDKTEVSLVWNRPDRDGGAEITGYLVEYQEDGADEWTKFQTVPMLDCVVTGL (SEQ ID NO: 44)
>Q90720|Q90720_CHICK Titin (Fragment)
VTTKQTVISRTREGIVTSQEQRHISREKVRKEPEKTPVPTVIIVTAKVKEQERISTAREEISARHEQVHVSHEQIRREDVKPSVP
KVVITTDKPKAPVLISQSKEGIATKKEHVSISHEKIKKEAKKTTAVLKIIAPVTVSRTREEITAKPEQMHLAYDQIEAGKRAEAVATVVAAVD
QARVRSPWETEQVDETYVKKKTLEYGYKEHAVKAHEAQAEHHVATKEVKTVYVPPEKHIPAAEKKEVHVSTEIKRETEAKIEKTNHIEHPRPR
TASPHFTVSKIAVPKPDHTYEVSIAGSAMATLEKELSATSAEQKITKPVKPPQLKPHEVKIKPESAPPPFPFTEAAETYKAHYDVETKKEVDV
SIKGGAVREDHLLLRKESEAKVTETARVPVPAEIPVTPTHLVWGLKNKTVTEGESVTLECHISGHPQPTVTWYRDDYKIESSMDFQITFKAGL
ARLVIREAFAEASGRFTCTATNKAGSVSTSCHLHVKVSEETETRETISEKVVTEEKSYVETQDVVMEDASPPPEEVSGEPVPPFFIRKPVVHT
LIVGGSIIFEFHVGGNPKPHVLLKKGGVPLTTGYRYKVSYKRETG (SEQ ID NO: 45)
>tr|F1N757|F1N757_BOVIN Titin OS = Bos taurus OX = 9913 GN = TTN PE = 4 SV = 2
MATQAPTFTQPLQSVVVLEGSTATFEAHISGFPVPEVSWFRDGQVISTSTLPGVQISFSDGRAKLTIPAVTKANSGRYSLRATNGSGQATSTA
ELLVTAETAPPNFTQRLQSMTVRQGSQVRLQVRVTGIPTPVVKFYRDGAEIQSSLDFQISQEGELYSLLIAEAYPEDSGTYSVNATNSVGRAT
STAELLVQGEEVVPAKRTKTIVSTAQISETRQTRIEKKIEAHFDARSIAAVEMVIDGAAGQQLPHKTPPRIPPKPKSRSPTPPSIAAKAQLAR
QQSPSPIRHSPSPVRHVRAPTPSPVRSVSPAGRISTSPIRSVKSPLLVRKAQTPTMPPGPEVPPPWKQEGYVASSEAEMRETTVTSATQIRTE
ERWEGRYGIQEQVTISGAAGAAASTTFAAGAVAAGAAEVKQEADKSAAVATVVAAVDMARVREPVISAVEQTAQRTTTTAVHIQPAHEQIRKE
AEKTAVTKVVVAADKAKEQEVKARTREVITTKQEQVHVTHEQIRKETEKAFVPKVVISAAKAKEKETKITGEITTKQEQKQITQETIIQKAET
AAVSMVVVETAKPTTLETILGAQEEIVTQQDQMHITHEKILKETRKTVVPKVIVATPKVKEQDVVSRTREGITTKREQVQVTHEKMRKEAEKT
ALSTIAVATAKAKEQETILRTREEMATRQEQIQVTHGKVGVGKKAEAVATVVAAVDQARVREPREPRHVEESYAQQTTLEYGYKEHISATKVA
EQPPRPASEPQVVPKAVKPGVIHAPSETHIATTDQMGMHISSQIKKTTDLTSERLVHVDKRPRTASPHFTVSKISVPKTEHGYEASIAGSAIA
TLQKELSATPSAQKVTKSVKAPTVKPAEARVRAEPTPSPQFPFAETPETFKSQAGIEVKKEVGVSITGVAVREERFEALRERETKVTETARVP
APVEIPVTPPTLVSGLKNVTVIEGESVTLECHISGYPSPKVTWYREDYQIESSIDFQITFQSGIARLLIREAFAEDSGRFTCTAVNEAGTVST
SCYLAVQVSEEFEKETTAVAEKITTEEKRFVESRDVVMTDTSITEEHAGPGEPAAPFFITKPVVQKLVEGGSIVFECQVGGNPKPHVYWKKSG
VPLTSGYRYKVSYNKQTGECRLVISMTFADDAGEYTIVIRNKHGETSASASLLEEADYESLMKSQQEMLYQTQVTAFVQEPKVGEIAPGYVYS
EYEKEYEKEQALIRKKMAKDTVMVRTFVEDQEFHISSFEERLIKEIEYRIIKTTLEELLEEDGEEKMAVDISESEAIEAGFDLRVKNYRILEG
MGVTFHCKMSGYPLPKIAWYKDGKRIKHGERYHMDFLQDGRASLRIPVVLPEDEGIYTAFASNMKGNAICSGKLYVEPAAPLSAPTYIPTPEP
VSRIRSISPRSVSRSPIRMSPARMSPARMSPGRRLEETDESQLERLYKPVFVLKPTSFKCVEGQTARFDLKVVGRPMPETFWFHDGQQIVNDY
THKVVVKEDGTQSLLIVPATPSDSGEWTVVAQNRAGKSSISVILTVEAVEHQVKPVFVEKLRNLNIKEGSRLEMKVRATGNPNPDIVWLKNSE
IIVPHKYPKIRIEGTKGEAALKIDSTVSQDSAWYTATAINKAGRDTTRCKVNVEVEFAEPEPERRLIIPRGTYRAKEIAAPELEPLHLRYGQE
QWEEGDLYDKEKQQKPFFKKKLTSLRLKRFGPAHFECRLTPIGDPTMVVEWLHDGKPLEAANRLRMINEFGYCSLDYAVAYSRDSGVITCRAT
NKYGTDHTSATLIVKDEKSLVEESQLPEGRKGLQRIEELERMAHEGALTGVTTDQKEKQKPDIVLFPEPVRVLEGETARFRCRVTGYPQPKVN
WYLNGQLIRKSKRFRVRYDGIHYLDIVDCKSYDTGEVKVTAENPEGVIDHKVKLEIQQREDFRSVLRRAPEPKPEFHVHEPGKLQFEVQKVDR
PVDTTETKEIVRLKRAERITHEKVSEESEELRSKFKRRTEEGYYEAITAVELKSRKKDESYEELLRKTKEELLHWTKELTEEEKKALAEEGKI
TIPTFKPDKIELSPSMEAPKIFERIQSQTVGQGSDAHFRVRVVGKPDPECEWYKNGVKIERSDRIYWYWPEDNVCELVIRDVTAEDSASIMVK
AINIAGETSSHAFLLVQAKQLITFTQELQDVVAKEKDTMATFECETSEPFVKVKWYKDGVEIHTGDKYRMHSDRKVHFLSVLMIDTSDAEDYS
CVLVEDENVKTTAKLIVEGAVVEFVKELQDIEVPESFSGELECIITPENIEGKWYHKGVELKSNGKYTITSRRGRQNLTVKDVTKEDQGEYSF
VVDGKKTTCKLKMKPRPIAILQGLSDQKVCEGDIVQLEVKVSLENVEGVWMKDGEEVQPGDRVHIVIDKQSHMLLIEDMTKEDAGHYSFTIPS
LGLSTHGRVSVYSVDVITPLKDVNVLEGTKAVLECKVSVPDVTSVKWYLNDEQIKPDDRVHAIVKGTKQRLVINRTHASDEGPYKLVVGRVET
SCDLSVEKIKIIRGLRDLTCTETQNVVLEVELSHSGIDVLWNFKDKEIKPSSKYKIEAHGKIYKLTVLNMMKDDEGEYTFYAGENRTSGKLTV
AGGAISKPLTDQTVAESQEAVFECEVANPDSEGEWLKDGKHLPLSKTIRSESDGRKRRLIIAATKLDDIGEYTYKVATSKTSAKLKVEAVKIK
KTLKNLTVTETQDAVFTVELTHPNVKGVQWIRNGVVLESSDKYTVSVKGTVYSLRIKNCAVADESVYGFKLGKLGASARLHVETVKIIKKPKD
VTALENATVSFEVSVSHDTVPVKWFHKNVEIKPSDKHRLVSERKVHKLMLQHISPSDAGEYTAVVGQLECKAKLFVETLHITKTMKNIEVPET
KTASFECEVSHFNVPSMWLKNGVEIEMSEKFKIVVQGKLHQLIIMNTSTEDSAEYTFVCGNDQVSATLKVTPIMITSMLKDINAEEKDTITFE
VTVNYEGISYKWLKNGVEIKSTDRCQMRTKKLTHSLNIRNVHFGDAAEYTFVAGKATSTATLYVEARHIEFRKHIKDIKVLEKKRAMFECEVS
EPDITVQWMKDGQELQLVDRIKIQKEKYVHRLLIPSTRMSDAGKYTVVAGGNMSSANLFVEGRDVRIRSIKREVQVIEKQRAVVEFEVNEDDV
DAHWYKDGIEINFQVQERHQYVVERRIHRMFISETRHSDAGEYTFVAGRNRSSVTLYVNAPEPPQILQELQPITVQSGKPARFCAVISGRPQP
KISWYKEEQLLSTGFKCKFLHDGQEYTLLLIEAFPEDAAVYTCEARNDYGVATTSASLSVEVPEVVSPDQEMPVYPPAIVSPLQDAVTSEGRP
AHFQCRVSGTDLKVSWYSKDKKIKPSRFFKMTQFEDTYQLEVAEAFPEDEGIYTFVASNAVGQVSSTATLRLEAPEAMLHEQIEMEMKEFSIS
LLSGKEERPQTSSWDLQLFDINETLEPLSEPSVYSPKFDSKKEGNAPVFIKEVSNAEISIGDVAKLFVTVTGIPTPQIQWFFNGAMLTPSADY
KFVFDGNDHSLIILFTKLEDEGEYTCIASNEYGQAMCSAYLKINSEAEGHEEPESAEKSLEKLQGPCPPYFLKELKPVHCVQGLPAIFEYVVL
GEPAPTVLWFKENKQLCTNVYYTIIHNPDGSGTFIVNDPQKEDGGLYVCKAENVWGGSTCAAELFVLLEDTDMTDATCKAAPTPEAPEGFPHT
SVKDPAVETFDSEQEVVTFVKDAILKASLIAEEKQQFSYEHVVKTNELSSQVILKAEQLQSTVILEHDMPTPESTRELLSISGTIHVQPIKEL
PPSLQLQIAQSQDTLPREDTLMCQEPESQVVLSDTEKIFPSFMSIEEISLSTVESLQTLLAEPEESYPQPLIKETPAHSYPTSVAEEVLLSKD
KTVSAVDRGQRETSQKQESQNALFLNQNLAEGHAQSLQGPDVTVSRVSSEHTCTESEILMESVDQLDSAGQDFAARIEEGQSLRFPLACEEKQ
VLLKEEHSDTLALPLNQTTEYKKEPMAVNGVQEVQGSDFLSKESLLPGIPEEQRLNLKTQIRSALQAAVASEQLSLFSEWLRNIEQVEVKAID
FTQEPNCIMCTYLITSVKSLTEEVTVTIKGIDPQMADLKTELKEALCSICEEMNILTAEEPGIEKGATVVLQEDVSFSSSQKLEAITEPEAES
KYLVSKEEISYFKVESQVKAVGTTTASAAVSDEKQDELPKPSEEKEKVSEGGTEEVGTVEIQEAEDEEDRKLGEDGPDVQTPLVDTIAEEGDI
ISLTSCITNAKEVNWYFESKLVPSDEKFKCLQDQNTYTLVIDKVNREEHQGEYTCEALNDNGKATTSAKLTVVKRAAPVIRRRIEPLEVALGH
LAKFTCEIHSAPNVRFQWFKAGREIYESDKCSIRSANYVTTLEILRTQVVDCGEYTCKASNEYGSVSCTATLTVTEAYPPTFFSRPKSVTTFV
GKAAKFLCTVTGTPVIETIWQKDGMALSPSPTCKISDVDNKHILELSNLTVHDKGVYSCKASNKFGADTCQAELDIIDKPHFIKELEPVQSAI
NKKIHLEGQVDEDRKVTITWSKNGQKLPPGKDYKIYFEDKIASLEIPLAKLKDSGTYVCTASNEAGSSSTSATVTIREPPSFVKKVDPSYLML
PGESARLHCRLKGSPVIQVTWFKNNKELTESNTIRMSFVNSEAVLDITDVKVEDSGTYSCEAVNDVGSDSCSAEIVVKEPPSFIKTLEPADIV
RGTNALLQCEIAGTGPFEISWFKDKKQIRSSKKYRLFSQKSTVSLEIFSFNSADVGDYECVVANEVGKCGCVATHLLKVEPPTFVKKVDDFTA
LAGQTVTLQAAVRGSEPISVTWMKGQEIIKEDGKIKMSFANGVAVLIIPDVQISFGDKYTCLAENEAGSQTSVGELIVKEPAKIIERAELIQV
TAGDPATLEYTVAGTPELKPKWYKDGRPLVASKKYRISFKNNVAQLKFYSAELHDSGQYTFEISNEVGSSSCETTFTVLDRDIAPFFTKPLRN
VDSVVSGTCRLDCKIAGSLPMRVSWFKDGKEVTSSDRYRIAFVEGTASLEISRVDMSDAGNFTCRATNSVGSKDCSGALIVQEPPSFVTKPAS
KDILPGSAVFLKSTFQGSTPFTIRWFKGDKELVSGGNCYINKEALESSLELYSVKTTDSGTYTCKVSNVAGAVECSANLFVKEPATFVERLEL
SQLLKKGDTTQLACKVTGTPPIKITWFANDREIKESSKHKMSFVESTAVLRLTDIAVEDSGEYMCEAQNEAGSDHCSSILIVKESPYFTKEFK
PIEVLKEYDVMLLAEVAGTPPFEITWFKDNTTLRSGRKYKTFIQDRLVSLQILKFVAADVGKYQCRVTNEVGSSTCSARVTLREPPTFVKKIE
STSSLRGGTAAFQATLKGSLPITVTWLKDNEEITEDDNIRMTFENNVASLYLSGIEVKHDGKYVCQAKNDAGIQRCFAVLSVKEPATIIEEAV
SIDVTQGDPATLQVKFSGTKEITAKWFKDGQELTLGQKYKISVTDTVSILKIISTEKRDSGEYTFEVQNDVGRSSCKASINVLDLIIPPSFTK
KLKKMDSIKGSSIDLECIVAGSHPISIQWFKDDQEITASEKYKFSFHDNTAFLEISQLEGSDSGTYTCSATNKAGHNQCSGHLTVKEPPYFLE
KPQSQDVNPNTRVQLKALVGGTAPMTIKWFKDNKELHSGAARSVWKDDTSTILELFSAKAADSGTYICQLSNDVGTATTKASLFVKEPPQFIK
KPSPVLVLRNGQSTTFECQITGTPEIRVSWYLDGNEITAVEKHGISFIDGLATFQISGARVENSGTYVCEAQNDAGTASCSIELKVKEPPTFI
RELKPVEVVKDSDVELECEVMGTSPFQVTWLRNNKEIRSSKKYTLTDRVSVENININRCDPSDTGDYQCIVSNEGGSCSCSARVSLKEPPSFI
KKIENITTVLKSSATFQSTVAGSPPISITWLKDDQILDEDDNVHISFVNNVATLQIRSVDNGHSGRYTCQAKNESGVERCYAFLLVQEPAQII
EKAKSVDVTERDPVTLECVVAGTPELKVKWLKDGKQIVPSRYFSMSFENNVASFRIQSVMKQDSGEYTFKVENDFGSSSCDAYLRVLDQNIPP
SFTKKLTKMDKVLGSSIHMECKVSGSLPISAQWFKDGKEISTSAKYRLVCHENTVSLDVNNLELEDTANYTCRVSNVAGDAACSGILTVKEPP
SFLVKPERQQAIPDSTVEFKAVLKGTPPFKIKWFKDDVELASGPKCFIGLEGSTSFLNLYSVDASKTGQYTCQVINDVGSDSCTTTLLVTEPP
KFVKKLEATKIVKAGDSARLECKITGSPDIRVVWYRNEHELPASDKYRMAFIDSVAVLQMNYLGTEDTGDFICEAQNPAGSTSCSTKVIVKEP
PVFSSFPPVVETLKNAEVSIECELSGTPPFEVVWYKDKRQLRSSKKYKIASKNFHASIHILNVDTLDIGEYHCKAQNEVGSDTCICIVKLKEP
PRFVSKLNSLTVVAGEPAELQASIEGTQPISVQWLKEKEEVVRESENIRITFVENVATLQFSKAEPANAGKYICQIKNDGGMRENMATLSFIS
EPAVIVEKAGSMTVTVGETCTLECKVAGTPELSVEWYKDGKLLTSSQKHKFSFYNKISSLKILSVEKQDAGTYTFQVQNNVGKSSCTAVVDVS
DRMVPPSFTRRLKDTIGVLGTSCILECKVAGSSPISVAWFHGKTKIVSGAKYQTTFSDNVCTLQLNSLDSSDMGSYTCVAANVAGSDECRAVL
AVQEPPSFVKEPEPLEVLPGKNITFTSVIRGTPPFKVGWFRGARELVKGDRCNIYFEDTVAELELFNVDISQSGEYTCVVSNNAGQTSCTTRL
FVKEPAVFVKKLSDHSVEPGKSIILESTYKGTLPISVTWKKDGFNITPSEKCSIVTTEKTCILEILNSTKRDAGRYSCEIENEAGRDVCEALV
STLEPPYFVTELEPLEASVGDSVSLQCQVAGSPEITVSWYKGDTKLRPTPEYRTYFINNVATLVENKVNINDSGEYTCKAENSIGTASSKTVF
RIQERQLPPSFARQLKDIEQTVGLPVTLTCRLNGSAPIQVSWYRDGVLLRDDENLQTSFVDNVATLKILQTDLSHSGQYSCSASNPLGTASSS
ARLTAREPKKSPFFDIKPVSIDIIAGESADFECHVTGAQPMRITWSKDNKEIRPGGNYTITCVGNTPHLRILKVGKGDSGQYTCQATNDVGKD
MCSAQLSVKEPPKFVKKLEASKVAKQGESIHLECKISGSPEIKVSWFRNDSELHESWKYNMSFVDSVAVLTINEASAEDSGDYICEAHNGVGD
ASCSTALTIKAPPVFTQKPSPIGALKGSDVVFQCEISGTPPFEVVWVKDRKQVRSSKKFKITSKNFDASLHILNLEAADVGEYYCKATNEVGS
DTCVCTLKFKVEPPRFVKKLSDTSTLVGDAVELRAVVEGFQPISVVWLKDKGDVIRESENTRISFVDNVATLQLGSPEASDSGKYVCQIKNDA
GMRECSAILTVLEPARIIEKPEPMTVTTGNAFALECVVTGTPELSAKWFKDGREIAADSKHHITFVNKVASLKIPCAEMSDKGLYSFEVKNSV
GKSTCTVSVHVSDRIVAPSFIRKLKDINAIVGASVVLECRVSGSAPISVGWFQDGNEIVSGPKCQSSFSENVCTLNLSFLEPSDTGTYTCVAA
NVAGSDECSAVLTIQEPPSFEQTPDSVEVLPGTSLTFTSVIRGTPPFKVKWFKGSRELVSGESCSISLEDFVTELELFEVEPLQSGDYSCLVT
NDAGSASCTAHLFVKEPATFVKRLADFSVETGSPIVLEATFTGTPPISVSWMKNEFALTQSQNCSITMTEKSTILEILDSTIEDYAQYSCLIE
NEAGQDICDAVVSVLEPPYFIEPLEHVEAVIGEPITLQCKVDGTPEIRISWYKEHTKLRSAPAYKMQFKNNVASLVINKVDHSDVGEYTCKAE
NIVGAVASSSVLVIKERKLPPSFARKLKDVQETLGFPVAFECRINGSEPLQVSWYKDGVLLKDDANLQTSFVHNVATLQILQTDQSHVGQYNC
SASNPLGTASSSAKLVLLEHEVPPFFDLKPVSVDLALGESGSFKCHVTGTAPMKITWAKDNREIRPGGNYKMTLVENTATLTVLKIGKGDAGQ
YTCYASNVAGKDSCSAHLGVQASIEPPRFIKKLEPSRIVKQDESTRYECKIGGSPEIKVLWYKGETEIQESSKFRMSFVDSVAVLEMHGLSVE
DSGDYTCEACNAAGSASSTTSLKVKALEPPIFRKKPHPVETLKGADVHLECELQGTPPFQVSWHKDKRELRSGKKYKIMSENFLTSIHILSVD
AADVGEYQCKATNDVGSDTCVGSITLKAPPRFVKKPSDVSTIVGEEVQLQATVEGAEPISVVWFKDKGEIVRESDNIWISYSENTATLQFSRA
ETANAGKYTCQIKNDAGMQECSATVSILEPAAIVEKPESITVTTGDTCTLECSVTGTPELTTKWFKDGKELTSDNKYKISFFNKVSGLKIINV
APSDSGVYSFEVQNPVGKDSCTASVQVSDSDRIVPPSFTRRLKETNGLSGSSVVMECKVYGSPPISVSWFHERNEISSGRKYQTTLTDNTCAL
TVNMLEESDAGNYTCIATNVAGSDECSAPLTVREPPSFVQKPDPMDVLTGTNVTFTSIVKGTPPFSVSWFKGSTELVPGDTCNVSLEDSVAEL
ELFDVDTSQSGEYTCIVTNEAGKVSCTTHLYVKAPARFVKKLNDYSIEKGKPLILEGTYTGTPPISVTWKKNGINVTPSQRCNITTTERSAIL
EIPSSTVEDAGQYNCYIENTSGKDSCSAQILILEPPYFVRQLEPVKVTVGDSASLQCQLGGTPEIAVSWFKGDTKLRPTATYKMHFRNNIATL
VFNQVDSNDSGEYICRAENSVGEVSSSTFLTVQEQKLPPSFSRQLRDVQETVGLPVVFECAVSGSEPISVSWFKDGRPVKDSPNIQASFLDNV
ATLNIFQTDRSFAGQYSCTATNPIGSASSSARLILTEGKNPPFFDIPLAPVDAVVGESADFECHVTGTQPIKVAWAKDNREIRSGGNYQISYL
ENSAHLTILKVDKGDSGQYTCYAVNEVGKDSCTAQLNIKERLIPPSFTKKLSETVEETEGNSFKLEGRVAGSQPISVAWYKNNIEIHPTSNCE
ITFKNNTLLLQIKRAGMDDAGLYTCKVSNDAGSALCTSSIVIKEPKKPPVFDQHLTPVTASEGEFVQLSCHVWGSEPIRIQWLKAGREIKPSD
RCSFSFANGTAVLELKDVTKADAGDYVCKASNVAGSDTSKSKVTIKDKPAAVPAAKKAAVDGKLFFVSEPQSIRVVEKTTATFIAKVGGDPIP
NVKWTKGKWRQLNQGGRILIHQKGDEAKLEIRDTTKTDSGLYRCVAFNKHGEIESNVNLQVDERKKQEKIEGDLRAMLKKTPVLKKGAGEEEE
IDIMELLKNVDPKEYEKYARMYGITDFRGLLQAFELLKQTEGEETHRLEIEEIEKSEKDEKEFEELVSFIQQRLSQTEPVTLIKDIENQTVLK
DNDAVFEIDIKINYPEIKLSWYKGTEKLEPSDKFEISIDGDRHTLRVKNCQLKDQGNYRLVCGPHIASAKLTVIATEPAWERHLQDVTLKEGQ
TCTMTCQFSVPNVRSEWFRNGRILKPQGRYKTEVEHKVHKLTIADVRAEDQGRYTCKHEDLETSAELRIEAEPIQFTKRIQNIVVSEHQSATF
ECEVSFDDAIVTWYKGPTELTESQKYNFRNDGRCHYMTIHNVTPDDEGVYSVIARLEPRGEARSTAELYLTTKEIKLEMKPPDIPDSRVPIPT
MPIRAIPPEEIPPTAVPPIPLLLPLPEEKKPPEKRVEVTKKAVKKDAKKVVAKPKEEAPPPKVIEVPKKPPRPTALIPAEAPEIIDVSSKAEE
VKITTITRKKEVQKEKEAVYERKQAVYEEKKVYIESLEEPYDELEVETYTEPYEEPYYEEPEEDYEETQVEAKREVHEEWEEDFEEGQEYYER
EEGYDEGEEEWEETYQEREVVQVQKEVYEESRERKIPAKVPEKKELPPPKVVKKPVVEKIEKTSRRMEEEKVQVTKVPEVSKKIVPQKPSRTP
VQEEIIEVKVPAVHTKKMVISEEKMFFAAHTEEEEEVSVTVPEVQKKTVTEEKVHVAVSKKIEPPPKVPEPPKKPEEVVPVPVPKKVEPPPAK
VPEVPKKPVPEEKKPVPVPKKEPAAPPKVPEVPKKPVPEEKVPVPIAKKKEAPPAKVPEVQKRVVTEEKITIVTEREESPPPAVPEIPKKKLP
EEKRPVPRKEEEVPPPKVPAVPKKPVPEEKVPVPVPVAKKAPPPRAEVSKKMVMEEKRFAAEEKLSVTVPQRVEVMRHEVSAEKEWRYSEEEE
RVSVSVYREEEREEEEVEVTEYEVMEEPEEYIVEEEEHFISEEVEAEPAEESFQVPEKKIIPKPKIPAKVEEPPPAKVPEAPKKIVPEKKIPA
AVPKKEKVPPTKVPEEPKKPVPEKRAPPKVAKIEEPPPTKVTERHMQITQEEKVHVAVTKKVEPPRPKVPEEPKRAVPEEKFPKLKPRKEEEP
PAKVTELRKRAVKEEKVSIEVAKREPPAAKEVTVTAEKEWAYTKEEEAVSVEREEEYEEYEEYDYKEFEEYEPTEEYDQYEEYEEREFEHYEE
YKEHEEYVTEPEKPVPVKPIQEEPVPTKPKAPPAKVPKKVIPEEKVPVPIPKKLKPPPPKAREEAKKVVEEKIQISVIKREKEQVTEPAAKVP
MKPKRVVTEEKVPVPKKEVAAPVRVPEVPKKEEPEEIAFEEEVVTHEEEYYEEEEEEEEYIHEEEEIITEEEVVPVKVFKVPEVPKKPVPEEK
KPVPVPKKKEAPPAKVPEIPKKPEEKVPVPVPKKEKAPPAKVPEVPKKPVPEEKVPVPEEKVPVPVPKKVEAPPAKVPEVPKKPVPEKKVPVP
APKKVEAPPAKVPEVPKKVLPEEKKPTPIRKKVEPPPPKVPKKREPVPVPIPAALLREEKVVHKEEIVVEEEEVLPEEEEISPEEEVPLEEEE
EEEEVLPEEEEVPPEEEEVPPEEEEVPPEEEEFVPEEEVVPEKEEVLPEIKPKVPVPARVPEVKKKVPEKKVIIPKKEEVPPAKVPEVPKKVE
EKRIIVPEEEEVPPVEVFEEPEEPIPEEEIPEEAPIVEEVEEEAPPRVPEVVKKAVPEAPTPVPKKVEVPPAKVPKKVPEEKPPVPVQKKEAP
PAKVPEVPKKVPEKKVPVPKKEVVPPAKGRAVLEEKVSVAFHKEEVVEERTELEIVEAQEEALEEEFHEVEEYFEEEEFHEVEEFLKVEKYRA
EEEVHRAEEVHRVIEVLEAEEEEAYEKPKAPPKGPEISEKVIPPKKPPTKVVPRKEPPAKVPEVPKKIVVEEKVHVPEEPKVAPAKVPEAPKE
VVPEKKVPAAPPKKPEVPPVTVPEAPKEVVPEKKVPVVPPKKPEVPPAKVPEVPKAAVPEKKVPEAIPPKPESPPPAVPEAPKEVVPEKKVPV
MPPKKPEVPPAKVPEAPKEVVPEKKVPVMPPKKPEVPPAKVPEVPKAAVPEKKVPEAIPPKPESPPPAVYEEPEEIAPEEPPVEVVEEPEPVP
APPPKVTVPPKKPVPEKKPPAVVAKKPEPPPAKVPEVPKEVVPEKKVPPVVPKKPEVPPPKVPEVPKEVVPEKKVAVPKKPEVPPAKVPEVPK
KPVLEEKPAIPIPEKVESPPPEEEEEPVPVVEEEEPVPVVEEEEPVPVVEEVEPEAPPPAVPEEPKKIVPEKKVPVIKKPEPPPPKEPEPEKK
VIEKPKLKPRPPAPPPAPPKEDVKEKIFQLKAVSKKKVPEKPAVPEKVELTPLKVAGGEKKVRKLLPEPKPQPKEEVILKSVLRKRPEEEEPK
VEPKKVEKIKKPAVPEPPPKAVEEVEAPPAVTKKERKIPEPTKVPEIKPPIPLPAPEPKPKPEAEVKIIKPPPVEPPPTPIAAPVTVPVVGKK
AEAKAPKEEAAKPKGPIKGVAKKTPSPIEAERKKLRPGSGGEKPPDEAPFTYQLKAVPLKFVKEIKDIVLTEAESVGSSAIFECLVSPSTAVT
SWMKDGSNIRESPKHRFIADGKDRKLHIIDVQLSDAGEYTCVLRLGNKEKTSTAKLIVEELPVRFVKTLEEEVTVVKGQPLYLSCELNKERDV
VWRKDGKIVVEKPGKIVPGVIGLLRALTINDADDSDAGTYTVTVENANNLECSSCVKVVEVIRDWLVKPIRDQHVKPKGTAVFTCVIAKDTPN
IKWYKGYDEIPWEPTDKTEIIRDGNHIHLKVKNAMPEDIDEYAVEIEGKRYPAKLTLGEREVELLKPIEDVTIYEKESASFDAEISEVDVPGQ
WKLKGELLRPSPTCEIKAEDGKRFLTLHNVKLDQAGEVLYQALNAVTTAILTVKEIELDFAVPLKDVTVPEKRQARFECVLTREANVIWSKGP
DIIKSSDKFDIITDGKKHILVINNSQFDDEGVYTAEVEGKKTSARLFVTGIRLKFISPLEDQTVKEGETATFVCELSHEKMHVVWFKNDAKLH
TSRTVLISSEGKTHKLEMREVTMDDISQIKAQVKDLSSTANLKVIEADPYFTVKLHDKTGVEKDEIVLRCEVSKDVPVKWFKDGEELLPSPKH
SIKADGLRRILKIKKADLKDKGEYVCDCGTDTTKANVTVEARLIKVEKPLYGVEVFVGETARFEIELSEPDVHGQWKLKGEPLTASPDCEIIE
DGKKHILVLYNCRLDMTGKVSFQAANAKSAANLKVKELPLIFITPLSDVKVFEKDEAKFECEVSREPKTYRWLKGTQEITGDDRIELIKDGTK
HSLVIKSAAFEDEAKYIFEAEDQHTSGKLIIEGIRLKFLTPLKDVTAKERESAVFTVELSHDNIRVRWFKNDQRLHTSKFVSMDDEGKTHSIT
FRNLSIDDTSQIKVEAMGLSSEAKLTVLEGDPYFTGKLQDYTGVEKEEVILQCEISKADAPVKWFKDGQEIKPSKNAVIKADGKKRMLILKKP
LKSDIGQYTCDCGTDKTSGKLNIEDREIKLVRPLYSVEVMETETARFETEISEDDIHANWKLKGEALLQTPECEIKEEGKTHFLILHNCRLDQ
TGGVDFQAANVKSSAHLRVKPRVIGLLRPLKDITVTAGETATFDCELSYEDIPVEWYLRGKKLEPSDKVVMRSEGRVHTLTLRDVKLEDAGEV
QLVAKDFKTQAKLFVKEPPVEFTKPLEDQTVEEEATAVLECEVSRENAKVKWFKNGTEILKSKKYEIVADGRVRKLIIHGCTPEDIKTYTCDA
KDFKTSCNLNVVPPHVEFLRPLTDLRVKEKEMARFECEISRENAKVHWFKDGAEIKKGKKYDIISKGAVRILVINKCLLDDEAEYSCEVRTAR
TSGMLTVLEEEAVFTKNLANIEVSETDTVKLVCEVSKPGAEVTWYKGDEEIIETGRYEILTDGRKRILIIQNAHLEDAGNYNCRLPSSRTDGK
VKVHELAAEFISKPQNLEILEGEKAEFVCSISKENFEVQWKRDDKTLESGDKYDVIADGKKRVLVVKDATLQDMGTYVVMVGAARAAAHLTVI
EKLRIIVPLKDTRVKEQQEVVFNCEVNTEGAKAKWFRNDEAIFDSSKYIILQKDLVYTLRIRNAQLDDQANYNVSLTNHRGENVKSAANLIVE
EEDLRIIEPLKDIETMEKKSVTFWCKVNRLNVTLKWTKNGEEVAFDNRVLYRIDKYKHSLIIKDCGFPDEGEYVVTAGQDKSVAELLIIEAPT
EFVEHLEDQTVTEFDDAVFSCQLSREKANVKWYRNGREIKEGKKYKFEKDGSIHRLIIKDCRLDDECEYACGVDDRKSRARLFVEEIPVEIIR
PPQDILEAPGADVVFLAELNKDKVEVQWLKNNMIIVQGDKHQMMSEGKIHRLQICDIKPRDQGEYRFIAKDKEARAKLELAAAPKIKTADQDL
VVDVGQPLTMVVPYDAFPKAEAEWFKENEPLSSKTVDTTAEQTSFRILKAKKEDKGRYKVVLQNKHGKAEGFINLKVIDVPGPVRNLEVTETF
DGEVSLAWEEPLTDGGSKIIGYVVERRDIKRKTWVLATDRADSCEFTVTGLQKGGVEYLFRVSARNRVGTGEPVETDSPVEARSKYDVPGPPL
NVTVTDVNRFGVSLTWEPPEYDGGAEITNYVIELRDKTSIRWETAMTVRAEDLSATVTDVVEGQEYSFRVRAQNRIGVGKPSAATPFIKVADP
IERPSPPVNLNASDQTQSSVQLTWEPPLKDGGSPILGYIIERCEEGKDNWIRCNKKLVPELTYKVTGLQKGNKYLYRVSAENEAGVSDPSEIL
GPLTADDADAEPTMDLSAFKDGLEVIVPNPIKILVPSTGYPRPTATWSFGDKVLEAGDRVKMKTLSAYAELVISPSERPDKGIYTLKLENRAK
AISGEIDVNVIARPSAPRELKFGDITKDSVHLTWEPPEDDGGSPLTGYVIEKREVSRKTWTKVIDSVTDLEFTVPDLLQGKEYLFKVCACNKC
GPGEPAYVDEPVNMSAPATVPDPPENVKWRDRTAKSIFLTWDPPKHDGGSRIKGYIVEKCPRGSDRWVACGEPVADTKMEVTGLEEGQWYAYR
VKALNRLGASKPSKPTEEIQAIDTQEAPEIFLDVKLLAGITVKAGTKIELPATVTGKPEPKITWTKADMLLKQDKRITIENVPKKSTVTIMDS
KRSDTGRYIIEAVNVCGRATAVVEVNVLDKPGPPAAFDITDVTNESCLLTWNPPRDDGGSKITNYVVERRATDSDMWHKLSSTVKDTKFKATK
LTPNKEYIFRVAAENMYGVGEPVQATPITAKFQFDPPGPPTRLEPSDITKDAVTLTWCEPDDDGGSPITGYWVERLDPESDKWVRCNKMPIKD
TTYRVKGLTNKKKYRFRVLAENLAGPGKPSKSTEPILIKDAIDPPWPPGKPVVKDIGRTSLMLNWTKPEHDGGAKIDSYVIEMLKTGTEDWVR
VAEGVPTTQHLLPGLMEGQEYSFRVRAVNKAGESEPSEPSDPVLCREKLYPPSPPRWLEVINITKNTADLKWTVPEKDGGSPITNYIVEKRDV
RRKGWQTVDTTVKDTKCTVTPLTEGSLYVFRVAAENAIGQSDYCEVEDSVLAKDTFTTPGPPYALAVVDVTKRHVDLKWEPPKNDGGRPIQRY
VIEKKEKLGTRWVKAGKTSGPDCHFRVTDVIEGTEVQFQVRAENEAGVGHPSEPTEILKIEDPTSPPSPPLDLHVTDAGRKHIAIAWKPPEKN
GGSPIIGYHVEMCPVGTEKWMRVNSRPIKDLKFKVEEGVVPDKEYVLRVRAVNAVGVSEPSEISENVVAKDPDCRPTIDLETHDIVVIEGEKL
SIPVPFRAVPVPTVSWHKDGKEVKASDRLTMKNDHISAHLEVPKSVRADAGIYTVTLENKLGSATASINVKVIGLPGPCRDLKASDVTKSSCK
LTWEPPEYDGGSPILHYVLERREAGRRTYIPVMSGENKLSWTVKDLIPNGEYFFRVKAVNKIGGGEYIELKNPVIAQDPKQPPDPPVDVEVHN
PTAEAMTITWKPPLYDGGSKIMGYIIEKIAKGEERWKRCNEHLVPVLTYTAKGLEEGKEYQFRVRAENAAGISEPSRATPPTKAVDPIDAPKV
IMKTSLEVRRGDEIALDAIISGSPYPTITWLKDESIITPEEIKKRVEPLVRRRRGEVQEEQPFVLPLTQRLSIDNSQKGESQLRVRDSVRPDH
GLFMIKAENDHGIAKAPCTVCVLDIPGPPINFVFEDMRKTSVLCKWEPPLDDGGSEILNYTLEKKDKTKPDSEWMVVTSTLRHCKYSVTKLIE
GKEYLFRVRAENRFGPGPPCVSKPFMAKDPFEPPDAPDKPIVEDVTSNSMLVTWNEPKDNGSPILGYWLEKREVNSTHWSRVNRNLLNSLKTK
VDGLLEGLTYVFRVCAENAAGPGKFSPPSDPKTAQDPISPPGPPVPRVTDTSSTTIELAWEPPAFNGGGEIVGYYVYKQLVGTNEWSRCTEKM
IKPREYTVREIREGADYKLRVTAVNVAGEGPPGETEPVTVAEPQAEPPTVELDVSVKGGIQIMAGKTLRIPAVVTGRPVPTKVWTIEEGELDK
DRVEIENVGTKSELIIKNALRKDHGRYVITATNSCGSKFAAARVEVFDVPGPVLDLKPVVTNRKMCLLNWSDPEDDGGSEITGFIIERKDAKM
HTWRQPIETERSKCDITGLLEGQEYMFRVIAKNKFGCGPPVEIGPILAVDPLGPPTSPERLTYTERTKSTITLDWKEPRSNGGCPIQGYIIEK
RRHDKPDFERVNKHLCPTTSFLVEDLDEHQMYEFRVKAVNEIGESEPSLPLNVVIQDDEVPPTIKLRLSVRGDTIKVKAGEPVNIPADVTGLP
MPKIEWSKNETVIEKPTDALKITKEEVSRSEAKTELSIPKATREDKGTYTVTASNRLGSVFRNVHVEVYDRPSPPRNLAVTDIKAESCYLTWD
APLDNGGSEITHYIIDKRDASRKRAEWEEVTNSAVERRYGIWKLIPNGQYEFRVRAVNKYGISDECKSDKVVIQDPYRTPGPPGKPKVLERTK
GSMLVSWTPPLDNGGSPITGYWLEKREEGGAYWSRVSRAPITKVGLKGVEFNVPRLIEGVKYQFRAMAINAAGVGPPSEPSDPEVAGDPIYPP
GAPSRPEVKDKTKSSITLAWKPPAKDGGSPIKGYIVEMQEEGTTDWKSVNEPDKLLPTCECVVPNLKELKKYRFRVKAVNEAGESEPSDTTGE
IPATDIQEVPEVFIDIGAQDCLICQAGTQIRIPAVIKGRPTPKSSWEFDGKAKKAMKDGVHDIPEDAQLETAENSSVIIIPECKRSHSGKYSI
TAKNKAGQKTANCRVKVMDVPGPPKDLKVSDITRGSCRLSWKMPDDDGGDRIKGYVIEKRTIDGKAWTKVNPNCVSTSFVVPDLIAGQEYFFR
VRAENRFGVGAPAETIQRTTARDPIYPPDPPIKLKIGLVTKNTVHLSWKPPKNDGGSPVTHYIVECLAWDPTGTKKEAWRQCNKRDVEELEFT
VEDLIEGGEYEFRVKAVNAAGVSKPSATVGPVIVKDQTCPPAIELKEFMEVEEGTDVNIVAKIKGVPFPTLTWFKAPPKKPDNKEPIVYDTHV
NKLVVDDTCTLVIPQSRRSDTALYTITAVNNLGTASKEMRLNVLGRPGPPVGPIKFESISADQMTLSWLPPIDDGGSKITNYVIEKREANRKT
WVRVSSEPKECTYTIPKLLEGHEYVFRIMAQNKYGIGEPLDSEPETARNLFSVPGAPDKPTVSSVTRNSMTVNWEEPEYDGGSPVTGYWLEMK
DTTTKRWKRVNRDPIKAMTLGVSYKVTGLIEGSDYQFRVYAINAAGVGPASLPSDPATARDPIAPPGPPFPKVTDWTKSSADLEWSPPLKDGG
SRVTGYIVEYKEEGKEEWEKAKDKEVRGTKLVVTGLKEGAFYKFRVRAVNIAGIGEPGEVTDAIEMKDRLELPDLQLDASVRDRIVVHAGGVI
RIIAYVSGKPPPTVTWNMNERALPQEATIETTAISSSMVIKNCQRSHQGVYSLLAKNAAGERKKTIIVDVLDVPGPVGIPFLAHNLINDSCKL
TWFSPEDDGGSPITNYVIEKREADHRAWTPVTYTVTRHNATVQGLIQGKAYFFRIAAENSIGMGPFVETTDALVIRDPITVPERPEDLEVKEV
TKESVTLTWNPPKYDGGSDIINYVLESRLIGTEKFHKVTSDNLMSRKYTVKGLKEGDTYEYRVSAVNIVGQGKPSFCTKPITCKDELAPPTLD
LDFRDKLTIRVGEAFALTGRYSGKPKPKVSWFKDEADVLEDDRTHIKTTPTTLALEKLKAKRSDSGKYSVVVENSTGSRKGVCQVNVVDRPGP
PVGPVIFDEVTKDYMVISWKPPLDDGGSEITNYIIEKKEVGKDVWMPVTSASAKTTCKVSKLLEGKDYIFRIHAENLYGISDPLVSDSMKAKD
RFRVPDAPDQPIVTEVTKDSALVSWNPPHDGGKPITNYILEKRETMSKIWARVTKEPIHPYTKFRVPDLLEGCHYEFRVSAENEIGIGDPSPP
SKPVFAKDPIAKPSPPVNPEAIDTTCNSVDLTWQPPRRDGGSKILGYIVEYQKVGDEEWKRANHTPESCPETNYKVTGLRDGQSYKFRVIAVN
AAGESDPAHVPEPVLVKDRLEPPELILDANMAREQHIRVGDTLRLSAIIKGVPFPKVTWKKEDRAAPTKARIDVTPVGSKLEIRNAAHEDGGI
YSLTVENPAGSKTVSVKVLVLDKPGPPRDLEVSEIRKDSCYLTWKEPLDDGGSVITNYVVERKDVASTEWSPLSTTSKKKSHLAKHLNEGNQY
VFRVAAENQYGRGPFVETPKPIKAVDPLHPPGPPKNLHHVDVDKTEVSLVWNKPDRDGGSPITGYLVEYQEEGTPDWIKFKTVTNLACVVTGL
QQGKTYRFRVKAENIVGLGLPDTTIPIECQEKLVPPSVELDVKLIEGLVVKAGTTVRFPAIIRGVPIPTAKWTTDGNEIKTDEHYTVETDNES
SVLTIKNCLRKDTGEYQITVSNAAGTKTVAVHLTVLDVPGPPTGPINILEVTPEYMTISWLPPKDDGGSPVINYIVEKKDTKKDTWGVVSSGS
SKTKLKVPHLQKGYEYVFRVKAENKIGIGPPLDSVPTVAKHKFSPPSPPGKPVVTDITENAATVAWTLPKSDGGSPITGYYLERREVTGKWVR
VNKTPLVDLKFRVTGLYEGNTYEFRVFAENLAGLSGPSPSSDPIKACRPIKPPGPPINPKLKDKTRESADLVWTKPLSDGGSPILGYVVECQK
AGTTQWDRINKDELIRQCAFRVPGLIEGNEYRFRIKAANIVGEGEPRELAESVIAKDILHPPEVELDVTCRDVITVRVGQTIRILARVKGRPE
PDITWSKEGKALVRDKRVNIIHELPRVELQIKEAVRADHGKYIISAKNSSGHAQGFAIVNVLDRPGPCQNLKVTNVTKENCTISWENPLDNGG
SEITNFIVEYRKPNQKGWSIVASDVTKRLIKANLLANNEYYFRVCAENKVGVGPTIETKTPILAINPIDRPGEPENLHIADKGKTFVYLKWRR
PDYDGGSPNLSYHVERRLKGAADWERVHKGSIKETHYMVDKCVENQIYEFRVQTKNEGGESDWVKTEEVVVKEDLQKPVLDLKLSGVLTVKAG
DTIRLEAGVRGKPFPEVSWTKDKDATDLTRSPRVKIDTSGDSSKFSLTKAKRSDGGKYVVTATNTAGSFVAFATVNVLDKPGPIRNLKITDVC
SDRCSLRWDPPEDDGGCEIQNYILEKCESKRMVWSTFSSTILTPGTTVTRLIEGNEYIFRVRAENKIGTGPPTETKPVIAKTKYDKPGRPDPP
EVTKVSKEEMTVVWAPPEYDGGKSITGYYLEKKEKHSTRWVPVNKSAIPERRLKVQNLLPGHEYQFRVKAENEVGIGEPSLPSRPVVAKDPIE
PPGPPTNLKVVDTTKSSITLGWGKPVYDGGAPIIGYVVEMRPKKADMSPDEGWKRCNAAAQLVRMEFTVTSLDENQEYEFRVCAQNQVGIGRP
AELKDAIKPKEILEPPEIDLDASMRKLVTVRAGCPIRLFAIVRGRPAPKVTWRKVGIDNVVRKGQVDLVDTMAFLVIPNSTRDDSGKYSLTLV
NPAGEKAVFVNVRVLDTPGPVSDLKVSDVTKTSCHISWAPPENDGGSQVTHYIVEKREAERKTWATVTPEVKKTSFHVTNLVPGTEYFFRVTA
VNEYGPGVPTDVPKPVLATDPLSEPDPPRKLEVTEMTKNSAVLAWLPPLRDGGAKIDGYIVSYREEEQPADRWTEYSVVKDLSLVVTGLKEGK
KYKFRVAARNAVGVSLPREAEGVYEAKEQLLPPKILMPEQITIKAGKKLRIEAHVYGKPNPVCKWKKGEDDVVTSSHLAVHKAENSSVLIIKD
VTRKDSGYYSLTAENSSGTDTQKIKVIVMDAPGPPQPPFDISDIDADACSLSWHIPLEDGGSNITNYIVEKCDVSRGDWVTALASVTKTSCRV
GKLIPGQEYIFRVRAENRFGISEPLTSPKMVAKFPFGVPSEPKNARVTKVNKDCIFVAWDRPDSDGGSPITGYLIERKERNSLLWVKANDTPV
RSTEYPCAGLVEGLEYSFRIYALNKAGSSPPSKPTEYVTARTPVDPPGKPEVIDVSKSTVSLVWARPKHDGGSKIIGYFVEACKLPGDQWIRC
NTSPHQIPQEEFTVTGLEENAQYQFRAIAKTAVNISQPSEPSDPVTIMAESVPPRIELSVAMKSLLTVKAGTNVCLDATVFGKPKPTVSWKKD
GTLLKPSEGIKMAMKRNLCTLELFSVTRKDSGDYTITAENPSGSKSATIKLKVLDKPGPPASVKINKMYSDRAMLSWEPPLEDGGSEITNYIV
DKRETSRPNWAQVSATVPITSCSVEKLIEGHEYQFRICAENKYGVGDPILTEPAIAKNPYDPPGRCDPPVISNVTKDHMTVSWKPPADDGGSP
ITGYLVEKRETHAVNWTKVNRKPVIERTIKATGLQEGTEYEFRVIAINKAGPGKPSDASKAVYAQDPLYPPGPPAFPKVYDTTRSSVSLTWGK
PAYDGGSPIIGYLVEVKRADSDNWVRCNLPQKLQKTRFEVTGLMENTEYQFRVYAVNKIGYSDPSDVPDKHCPKDILIPPEGELDADLRKTLI
LRAGVTMRLYVPVKGRPPPKITWSKPNVNLRERVGLDIKSTDFDTFLRCEKVNKYDAGKYILTLENSCGKKEYTIVVKVLDTPGPPVNVTVKE
ISKDSAYITWDPPIIDGGSPIINYVVEKRDAERKSWSTVTTECSKTSFRVSNLEEGKSYFFRVFAENEYGIGDPGETRDAVKASETPGPVVDL
KVTSVTKSSCSIGWKKPRSDGGSRIIGYVVDFLTEENKWQRVMKSLSLQYTTKDLTEGKEYTFRVSAENENGEGTPSEITAVAKDDVVAPDLD
LKDLPDLCYLAKENSNFRLKIPIKGKPVPSVSWKKGEDPLATDTRVSVESSAVNTTLVVYDCRKSDAGKYTITLKNVAGTKEGTLTIKVVGKP
GIPTGPIKFEEVTAEAVTLKWGPPKDDGGSEITNYILEKRDSVNNKWVTCASAVQKTTFRVMRLHEGMEYTFRVSAENKYGVGEGLKSEPIVA
RHPFDVPDAPPPPNIVDVRHDSVSLTWTDPRKTGGSPITGYHIEFKERNSLLWKRANKTPIRMKDFKVTGLTEGLEYEFRVMAINLAGVGKPS
LPSEPVVALDPIDPPGKPEVISVTRNSVTLVWTEPKYDGGHKLTGYIVEKRELPSKSWTKANHVNVPDCAFTVTDLVEGGKYEFRVRAKNTAG
AISAPSESTDTIICKDEYEAPTIVLDPTIKDGLTVKAGDTIILSAISILGKPLPKSSWSKAGKDIRPSDIVQITSTPTSSMIAVKYATRKDAG
EYTITATNPFGTKSEHVKVTVLDVPGPPGPIEISNVSAEKATLTWTPPLEDGGSPIKSYVLEKRETSRLLWTVVAEDIQSCRHVATKLIQGNE
YVFRVSAVNQYGKGEPVQSEPVKMVDRFGPPGPPGKPEVSNVTKNTATVSWKRPVDDGGSEITGYHVERREKKSLRWVRATKTPVSDLRCKVT
GLQEGNTYEFRVSAENKAGIGPPSDASNPVLMKDVAYPPGPPSNARVTDTTKKSASLAWGKPHYDGGLEITGYVVEHQKVGDEGWIKDTTGTA
LRITEFVVPDLETKEKYNFRISAINDAGVGEPAVIPNVEIVEREMAPDFELDAELRRTLVVRAGLSIRIFVPIKGRPAPEVTWTKDNINLKNR
ANIENTESFTLLIIPECNRYDTGKFVMTIENPAGKKSGFVNVRVLDTPGPVLNLRPTDITKESVTLHWDLPLIDGGSRITNYIVEKREATRKS
YSTVTTKCHKCTYKVTGLSEGCEYFFRVMAENEYGIGEPAETTEPVRASEAPSPPDSLNIMDITKSTVSLAWPKPKHDGGSKITGYVIEAQRK
GSDQWTHITTVKGLECVVKNLTEGEEYTFQVMAVNSAGRSAPRESRPVIVKEQTMLPELDLRGIYQKLVIAKAGDNIKVEIPVLGRPKPTVTW
KKGDQILKQTQRVNFENTATSTILNINECVRSDSGPYPLTARNIVGEVGDVITIQVHDIPGPPTGPIKFDEVSSDFVTFSWEPPENDGGVPIS
NYVVEMRQTDSTTWVELATTVIRTTYKATRLTTGVEYQFRVKAQNRYGVGPSITSAPVVANYPFKVPGPPGTPQVAAVTKDSITISWHEPLSD
GGSPILGYHVERKERNGILWQTVSKALVPGNIFKSSGLTDGIAYEFRVIAENMAGKGKPSKPSEPILALDPIDPPGKPVPLNITRHTVTLKWA
KPEYTGGFKITSYIVEKRDLPNGRWLKANFSNILENEFTVSGLTEDAAYEFRVIAKNAAGAISPPSEPSDAITCRDDIEAPRIMVDVKFKDTI
TLKAGEAFKLEADVSGRPPPTMEWTKDGKELENTAKLEIKIADFSTNLVNKDSLRRDGGAYTLTATNPGGFAKHIFHVKVLDRPGPPEGPLAV
SEVTSEKCVLSWLPPLDDGGAKIEYYVVQKRETSRLAWTNVASEVQVTKLKVTKLLKSNEYIFRVMAVNKYGVGEPLDSEPVLAVDPYGPPDP
PKNPEVTSITKDSMVVCWGHPDSDGGSEIINYIVERRDKAGQRWVKCNKKTLTDLRYKVSGLTEGHEYEFRVMAENRAGISAPSATSPFYKAC
DAVFKPGPPGNPRVLDTSRSSISIAWNKPIYDGGSEITGYMVEIALPEEDEWKIVTPPSGLKATSYTITNLTENQEYKIRIYAMNSEGIGEPA
LVPGTPKAEDRMLPPEIELDAELRKVVTIRACCTLRLFVPIKGRPAPEVKWTREHGESLDKASIESTSSYTLLIIGNVNRFDSGKYILTIENS
SGSKSAFVNVRVLDTPGPPQDLKVKEVTKTSVTLTWEPPLIDGGSKINNYIVEKRESTRKAYSTVTTNCHKTSWKVDQLQEGCSYYFRVLAEN
EYGIGLPAETAESVKASERPLPPGKITLVDVTRNSVSLSWEKPEHDGGSRILGYIVEMQSKGSDKWVTCATVKVTEATITGLIQGEEYSFRVS
AQNEKGISDPRQLSVPVIAKDLVIPPAFKLLFTTFTVLAGEDLKVDVPFTGRPTPAVTWHKDDVPLKQTTRVNAESTENNSLLTIKEACREDV
GHYIVKLTNSAGEATETLNVIVLDKPGPPTGPVKMEEVTADSVTFSWGPPKYDGGSSINNYIVEKRDTSTTTWQIVSATVARTTIKACRLKTG
CEYQFRIAAENRYGKSTYLTSEPVVAQYPFKVPGPPGTPFVTLSSKDSMEVHWNEPVSDGGSKVIGYHLERKERNSILWVKLNKTPIPQTKFK
TTGLDEGIEYEFRVSAENIVGIGKPSKVSESYVARDPCDPPGRPEPIIVTRNSVTLQWKKPAYDGGSKITGYIVEKKELPDGRWMKASFTNVI
DTQFEVTGLVEDHRYEFRVIARNAAGVFSEPSESTGAITARDEVEPPQIRMDPKYKDTIVVHAGELFKIDADIHGKPIPTTQWIKGDHELSNT
ARLEIKSTDFATSLSVKDAIRIDSGSYILKAKNVAGEKSVTVNVKVLDRPGPPEGPIVISGVTAEKCTLAWKPPLQDGGSDIINYIVERRETS
RLVWTVVDANVQTLGCKVTKLLEGNEYIFRVMAVNKYGVGEPLESEPVTAKNPFVVPDAPKAPEITAVTKDSMIVVWERPAFDGGSEILGYVL
EKRDKEGIRWTRCHKRLIGELRLRVTGLLENHNYEFRVSAENAAGLSEPSPPSAYQKACDPIYKPGPPNNPRVTDITRSSVFLSWGKPIYDGG
CEIQGYIVEKCDTSVGEWTMCTPPTGINKTNIEVEKLLEKHEYNFRICAVNKAGVGEHADVPGPVIVEEKLEAPDIDLDLELRKILNIRAGGS
LRLFVPIRGRPTPEVKWGKVDGEIRDAAIIDSTSSFTSLVLDNVNRYDSGKYTLTLENSSGTKSAFVTVRVLDTPSPPVNLKVTEITKDSVSI
TWEPPLLDGGSKIKNYIVEKREATRKSYAAVVTNCHKNSWKIDQLQEGCSYYFRVTAENEYGIGLPAHTDDPVKVAEVPQPPGKITVDDVTRN
SVSLSWTKPEHDGGSKIIQYIVEMQAKHSEKWSECARVKSLEAVITNLTQGEEYLFRVVAVNEKGRSDPRSLAVPIVAKDLVIEPDVRPAFNS
YSVQVGQDLKIEVPISGRPKPTITWTKDDLPLKQTTRINVTDSLDLTVLSIKETHKDDGGHYGITVANVVGQKTASIEIITLDKPDPPKGPVK
FDEVSAESITLSWEPPLYTGGCQITNYVVQKRDTTTTVWEVVSATVARTTLKVTKLKTGTEYQFRIFAENRYGQSFALESEPIVAQYPYKEPG
PPGTPFVTAVSKDSMVVQWHEPINNGGSPVIGYHLEKKERNSILWTKVNKTIIHDTQFKAVNLEEGIEYEFRVYAENIVGIGKASKNSECYVA
RDPCDPPGTPEAIIVKRHEITLQWTKPAYDGGSVITGYIVEKRDLPEGRWMKASFTNVIETQFTVSGLTEDQRYEFRVIAKNAAGAVSKPSDS
TGPITARDEVELPRISMDPKFRDTIVVNAGETFRLEADVHGKPLPTIEWLRGDKEIEESARYEIKNTDFKALLIVKDAIRIDGGQYILRASNV
AGSKSFPVNVKVLDRPGPPEGPVQVTGVTAEKCTLTWSPPLQDGGSDISHYVVEKRETSRLAWTVVASEVVTNSLKVTKLLEGNEYIFRIMAV
NKYGAGEPLESAPVLMKNPFVLPGPPKSLEVTNIAKDSMTVCWNRPDSDGGSEIIGYIVEKRDRSGIRWIKCNKRRITDLRLRVTGLTEDHEY
EFRVSAENAAGVGEPSPATLYYKACDPVFKPGPPINAHVVDTTKNSITLAWGKPIYDGGSEILGYVVEICKADEEEWQIVTPQTGLKATRFEI
SKLTEHQEYKVRVCALNKVGLGEATAVPGTVKPEDKLEAPELDLDSELRKGIVVRAGGSVRIHIPFKGRPTPEITWSREEGEFTDKVQIEKAA
NYTQLSIDNCDRNDAGKYVLKLENSSGSKSAFVTVKVLDTPGPPQNLAVKEVRKDSVLLVWDPPLIDGGAKVKNYVIDKRESTRKAYANVSNK
CSKTSLKVENLTEGAIYYFRVMAENEFGIGVPVETVDAVKASEPPSPPGKVTLTDVSQTSASLMWEKPEHDGGSRILGYVVEMQPKGTEKWSV
VAESKVCSAVVTGLSSGQEYHFRVKAYNEKGQSDPRVLGVPVIAKDLTIQPSFKLPFNTYSVQAGEDLKIEIPVIGRPRPEISWVKDGEPLKQ
TTRVNVEKTATSTILHIKESNKDDFGKYTVTATNSAGTATENLSIIILEKPGPPVGPVRFDEVSADFVVISWEPPAYTGGCQISNYIVEKRDT
TTTTWHMVSATVARTTIKITKLKMGSEYQFRIFAENRYGKSAPLDSKPVIVQYPFKEPGPPGTPFVTSVSKDQMLVQWHEPVNDGGSKVIGYH
LEQKEKNSILWVKLNKTPIQDTKFKTTGLDEGLEYEFRVSAENIVGIGKASKVSECFVARDPCDPPGRPEAIVITRNNVTLKWKKPAYDGGSK
ITGYIVEKKDLPDGRWMKASFTNVLDTEFTVSGLVEDQRYEFRVIARNAAGNESEPSESTGAITARDEIDAPNASLDPKYKDVIVVHAGETFV
LEADIRGKPIPDIVWSKDGRELEETAARMEIKSTIQRTTLVVKDCIRSDGGQYILKLSNYGGTKTIPITVKVLDRPGPPEGPLKVSGVTAEKC
YLAWNPPLQDGGASISHYIIEKRETSRLSWTQVSTEVQALNYKVTKLLPGNEYIFRVMAVNKYGTGDPLESEPVIARNPYKPPGPPSPPEVSA
ITKDSMVVTWARPVDDGGAEIEGYILEKRDKEGVRWTKCNKKTLTDLRFRVTGLTEGHSYEFRVAAENAAGVGEPSEPSVFSRACDALYPPGP
PSNPKVTDTSRSSVSLAWNKPIYDGGAPVKGYVVEVKEATADEWTTCTPPTGLQGKQFTVTELKENTEYNFRICAINSEGVGEPATIPGSVAA
KERQEPPEIELDADLRKVVILRASATLRLFVTIKGRPEPEVKWEKAEGVLTDRAQIEVTSSYTMLVIDNVTRFDSGRYNLTLENNSGSKTAFV
NVRVLDSPSAPVNLTVREVKKDSVILAWEPPLIDGGAKITNYIVEKRETTRKAYATVINNCTKNSFKIENLQEGCSYYFRVLASNEYGIGLPA
ETTEPVKASEPPLPPGRVTLVDVTRNTATIKWEKPESDGGSKITGYVVEMQTKGSEKWSICTQVKTLEATISGLTAGEEYIFRVAAINEKGKS
DPRQLGVPVIARDIEIKPSVELPFNTFNVKARDQLKIDIPFKGRPQATVSWKKDGQTLKETTRVNVSSSKTVTSLIIKEASREDVGTYELCVS
NSAGSVTVPITVIVLDKPGPPGPIHIDEVSCDNITISWNPPEYDGGCQISNYIVEKRETTSTTWHVVSQAVARTSIKIVRLVTGSEYQFRVCA
ENRFGKSSYSESSAVVAEYPFSPPGPPGTPKVVHATKSTMLVTWQVPVNDGGSRVLGYHLEYKERSSILWSKANKTLIADTQMKVSGLDEGLM
YEYHVYAENIAGIGKCSKSCEPVPARDPCDPPGQPEVTNITRKSVSLKWSKPHYDGGAKITGYIIERRELPDGRWLKCNFTNVPETYFEVTEL
TEDQRYEFRVFARNAADSISEPSESTGPITVKDDVEAPRIMMDVKFRDVIVVKAGEVLKINADVAGRPLPIISWAKDGVEIEERARTEIVSTD
YTTLLTVKDCVRRDSGQYVLTVKNVAGTRSMAVNCKVLDKPGPPAGPLEITGLTAEKCSLSWGPPQEDGGAAIDYYIVEKRETSRLAWTICEG
ELRTTFCKVTKLLKGNEYIFRVTGVNKYGVGEPLESMAVKALDPFTVPSPPTSLEITSVTKEFMTLCWSRPESDGGSEISGYIVERREKNSLR
WVRVNKKPVYDLRVKSTGLREGCEYEYRVYAENAAGLSLPSESSPLIRAEDPVFLPSPPSKPNVMDSGKTNITIGWVKPLFDGGAPITGYTVE
FKKSDETDWQTAIQNLRGTEYTVSGLTTGAEYVFRVRSINKVGASDPSDSSDPQIAKEREEEPVFDLDTEMRKTLIVKAGASFTMTVPFRGRP
VPSVSWSKPDTDLRTRAYIDSTESRTSLTIENANRNDSGKYTLMIQNVLNAASLTLVVKVLDSPGPPANITVHDVTKESAVLSWDVPENDGGA
PVKNYHIEKREASKKAWVSVTNNCNRLSYKITNLQEGAIYYFRVSGENEFGVGVPAETKEGVKITEKPSPPEKLGVTSVSRDSVSLAWLKPEH
DGGSRIVHYVIEALEKGQTTWMRCAVVKSTHHVVSGLRQNSEYFFRVFAENQAGLSDPRELLLPVLIKEQLEPPEIDMKNFPSHTVYVRAGSN
LKVDIPISGKPLPKVTLSRDGVPLKATMRENTEITAENLTINLKESVAADAGRYEITAANSSGTTKAFINIVVLDRPGPPTGPVVISDITEDS
VTLKWEPPKYDGGSQVTNYIVLKRETSTAVWTEVSATVARTMIKVMKLTTGEEYQFRIKAENRFGISDHIDSACVVVKLPYTTPGPPSTPWVT
NVTRESITVGWHEPVSNGGSPVTGYHLEMKDRNSILWQKANKMVIRTTHFKVTTISAGLIYEFRVYAENAAGIGKPSHPSEPVLAIDACEPPR
NVRITDISKNSVNLSWQQPAFDGGSKITGYMVERRDLPDGRWAKASFTNVTETHFTVSGLTQNAQYEFRVFARNAVGSISNPSEVVGPITCID
SYGGPVIDLPLEYTEVVKYRAGTSVKLRAGISGKPEPTIEWYKDDKELQTNALVCVENTTDLASILIKDATRLNSGTYELKLRNALGSASATI
RLQILDKPGPPGGPIEFKTVTAEKITLLWQPPADDGGAKITHYIVEKRETSRVVWSMVSENLEECIIKTTKIIKGNEYIFRVRAVNKYGIGDA
LESHPVIARNAFVTPGPPSVPEVTKINKNSMTVVWSRPVADGGSDISGYILEKRDKKSLGWFKVLKETIRDTRQKVTGLTEHSDYQYRVCAVN
AAGQGPFSEPSDFYKAADPIDPPGPPAKIRIADSTKSSITLGWSKPVYDGGSAVTGYVVAMRQGEEEEWTVVSTKGEVRTTEYVVSNLSPGVN
YYFQVSAVNCAGQGEPIAMTEPVQAKDILEEPEIDLDVAFRTSIIAKAGEDVHALVPFKGRPPPTVTWRKDEKNLGSDARYSIQNTDSSSILT
IPQVTRHDTGKYILTIENGVGQPKSSTVSVKVLDTPAACQNLQVKHVSQGTVTLFWDPPLIDGGSPIINYIIEKKDATKRTWSSVSHKCSSTS
FKVTDLSEKTPFFFRVFAVNEIGIGEPCETTEPVKAAEVPAPIRDLSVKDSTKTSVTLSWTKPDFDGGSVITDYIVERKGKGEQTWSHAGISK
TCEIEVGKLKELSELEFRVSARNEKGLSDSVSIGPVTVKELVIPPEVDLSEIPGAQVAVRIGHNVHLELPYKGKPKPSISWLKDGLPLKESEL
VRLGKTENKLTLSIKNAKKENGGKYTIILDNAVCRNTYPITVITLGPPSKPKGPIRFDEIKADSVIMSWDVPDDDGGGEITCYSIEKREASQT
NWKMVCSSVARTTFKVPNLVKDAEYQFRVRAENRYGVSQPLVSNIILAKHQFRIPGPPGKPVTYNVTSDGMSLTWDAPVYDGGSEVTGYHVEK
KERNSILWQRINMSPISGREYRATGLMEGLDYQFRVFAENSAGLSSPSDPSKFTLAVSPVDPPGTPDYIDVTRETITLKWNPPLRDGGSKIVA
YSIEKRQGSDRWTRCNFTDVSECQYTVTGLSPGDRYEFRIIARNAVGTISPPSQSSGVIMTRDENVAPTVEFGPEYFDGITIKSGESLRIKAL
VQGRPVPRVTWFKDGVEIEKKMNMEITDVLGSTSLFVRDATRDHRGVYRVEAKNASGSTKAEITVRVQDTPGKPVGPIRFTNITGEKMTLWWD
APHNDGCAPVSHYLIEKRETSRLAWALIEDNCEALSYTATKLITGNEYQFRISAVNKFGVSRPLDSDPVVAQIQYTIPDAPGTPEPSNVTGNS
ITLTWARPESDGGSEIQQYILERREKKSTRWVKVISKRPICETRFKVTGLTEGNEYEFHVMAENAAGVGPASGVSRLIKCREPVNPPGAPTVV
KVTDTSKTTVSLEWSKPVFDGGLEIIGYIIEMCKADLGDWHKVNVEACVKTRYTVTDLQAGEEYKFRVSAINAAGKGDSCEVTGTIKAVDRLS
APELDIDANFKQTHVVRAGASIRLFIAYQGRPTPTAVWSKPDSNLSIRADIHTTDSFSTLTVENCNRTDAGKYTLTVENNSGSKSITFTVKVL
DSPGPPGPITFKDVTRGSATLMWDAPLLDGGARIHHYVVEKREASRRSWQVVSEKCIRQILRVSDLLEGVPYYFRVSAENEYGVGEPYEMPEP
MVATEQPAPPRRLDIVDTSKSSVVLAWLKPDHDGGSRITGYLLEMRQKGSDFWVEAGHTKQMTFTVERLVENTEYEFRVKAKNDAGYSEPREA
FSSVIIKEPQIEPTADLTGITNQLITCKAGSTFTIDVPISGRPAPKVTWKLEEMRLKETDRVSITTTKDRTTLSVKDSMRGDSGRYFLTLENT
AGVKTFTITVVVIGRPGPVTGPIEVSSISAESCVLSWAEPQDNGGTDITNYIVEKRESGTTAWQLVNSSVKRTQIKVTHLTKYMEYSFRVSSE
NRFGVSKPVESAPIVAEHPFVPPSAPTRPEVYYVSANAMSIRWEEPYHDGGSKVIGYWVEKKERNTILWVKENKVPCLECNYKVTGLVEGLEY
QFRTYALNAAGVSKASEASRPVMAQNPVDAPGRPEVTDVTRSTVSLIWSAPVYDGGSKVVGYIIERKPVSEVGDGRWLKCNYTIVSDNFFTVT
ALSEGDTYEFRVLAKNAAGVISKGSESTGPITCRDEYAPPKAELDARLQGDLVTIRAGSDLVLDAAVGGKPEPKIIWTKGDKELDMCEKISLQ
YTGKRATAVIKFCDRSDSGKYTLTVKNASGTKAVSVLVKVLDSPGPCGKLTVSRVTEEKCTLAWSLPQEDGGAEITHYIVERRETSRLNWVIV
EGECPTLSHVVTRLIKNNEYIFRVRAVNKYGPGVPVESEPIVARNSFTIPSQPGIPEEVGAGKEHIIIQWTKPESDGGNDISNYLVDKREKKS
LRWTRVNKDHVVYDTRLKVTGLMEGCDYQFRVTAVNAAGNSEPSEASNFISCREPSYTPGPPSAPRVVDTTKHSISLAWTKPMYDGGTDIIGY
VLEMQEKDTDQWYRVHTNATIRNNEFTVTDLKMGQKYSFRVAAVNVKGMSEYSESTAEIEPVERLEIPDLELADDLKKTVTIRAGASLRLMVS
VSGRPPPVITWSKKGIDLASRAIIDTTESYSLLIVDKVNRYDAGKYTIEAENQSGKKSATVLVKVYDTPGPCPSVKVKEVSRDSVTITWEVPT
IDGGAPVNNYIIEKREAAMRAFKTVTTKCSKTLYRISGLIEGAMYYFRVLPENIYGIGEPCETSDAVLVSEVPLVPTKLEVVDVTKSTVTLAW
EKPLYDGGSRLTGYVLEACRAGTERWMKVVTLKPTVLEHTVISLNEGEQYLFRVRAQNEKGVSEPREIVTAVTVQDLRVLPTIDLSTMPQKTI
HVPAGRPVELVIPIAGRPPPAASWFFAGSKLRESERVTVETHTKVAKLTIRETTIKDTGDYVLELKNVTGTTSETIKVIVLVDKPGPPSGPIK
VDEIDATSVTISWGPPELDGGAPLSGYVVEQRDAHRPGWLPVSESVTRSTFKFTRLIEGNEYVFRVAATNRFGIGSYLQSEVIECRSSINIPG
PPETLQIFDVSREGMTLTWYPPEDDGGSQVTGYIVERKEVRADRWVRVNKVPVTMTRYRSTGLIEGLEYEHRVTAVNVRGTGKPSRPSKPTVA
MDPIAPPGKPQNPRVTDTTRTSVSLAWSVPEDEGGSKVTGYLIEMQKVDQHEWTKCNTTPTKIREYTLTHLPQGAEYRFRVLACNAGGPGEPA
EVPGTVKVTEMLEYPDYELDERYQEGILVRQGGVIRLTIPIKGKPFPICKWTKEGQDISKRAMIATSETHTELVIKEADRDDSGTYDLILENK
CGKKAVYIKVRVIGNPNTPEGPLEYDDIQARSVRVSWRPPADDGGADILGYILERREVPKSAWYTIDSRVRGTSLVVKGLKENVEYHFRVSAE
NQFGISKPLKSEEPVIPKTPLNPPEPPSNPPEILDVTKSSVSLSWSRPKDDGGSRVTGYYIERKETSTDKWVRHNKTQITTTMYTVTGLVPDA
EYQFRIIAQNDVGLSETSPASEPVVCKDPFDKPSQPGELEILSISKDSVTLQWEKPECDGGKEILGYWVEYRQSGDSAWKKSSKDRIKDRQFT
IGGLLEATEYEFRVFAENETGLSRPRRTAMSVKTKLTSGEAPGVRKEMKDVTTKLGEAAQLSCQIVGRPLPDIKWFRFGKELVQSRKYKMSSD
GRTHTLTVMTEEQEDEGVYTCVATNEVGEVESSSKLLLQATPQFHPGYPLKEKYYGAVGSTLRIHVMYIGRPVPAITWFRGQKLLQNSENITI
ENTEHYTHLVMKNVQRKTHAGKYKVQLSNVLGTVDTMLDVEIQDKPDKPTGPIVIEALLKNSVVISWKPPADDGGSWITNYVVEKCEAKEGAE
WQLVSSAISVTTCRIVNLTENAGYYFRVSAQNMFGISEPLEVSSVVIIKSPFEKPGAPGKPTITAVTKDSCVVAWKPPASDGGAKIRNYYLEK
REKKQNKWIAVTTDEIRETVFSVQNLIEGLEYEFRVKCENLGGESEWSEISEPVTPKSDVPIQAPHFKEELRNLNVRYQSNATLVCKVTGHPK
PIVKWYRQGKEIIADGLKYRIQEFKGGYHQLIIASVTDDDATVYQVRATNQGGSVSGTASLEVEDTVPAKIHLPKNLEGMGAVHALRGEVISI
KIPFSGKPDPVITWQKGQDLIDNNGHYQVIVTRSFTSLVFPNGVERKDAGFYVVCAKNRFGIDQKTVELDVADVPDPPRGVKVSDVSRDSVNL
TWTEPASDGGSKVTNYIVEKCATTAERWIRVGQARETRYTVINLFGKTSYQFRIIAENKFGLSKPSEPSEPTVTKEDKTRAMNYDEEVDETRE
VSMTKASHSSTKELYEKYMIAEDLGRGQFGIVHRCVETSSKKTYMAKFVKVKGTDQVLVKKEISILNIARHRNMLYLHESFESMEELVMIFEF
ISGLDIFERINTSAFELNEREIVSYVRQVCEALEFLHSHNIGHFDIRPDNIIYQTRRSSTIKIIEFGQARQLKPGDNFRLQFTAPEYYAPEVH
QHDVVSTATDMWSLGTLVYVLLSGINPFLAETNQQIIENIMNAEYTFDEEAFQEISLEAMDFVDRLLVKERKSRMTASEALQHPWLKQKTERV
STKVIRTLKHRRYYHTLVKKDLNMVVSAARISCGGAIRSQKGVSIAKVKVASIEIGPVSGQIMHAVGEEGGYVKYVCRIENYDQSTQVTWYFG
VRQLENSEKYEITYEDGVATMYVKDITKFDDGTYRCKVVNDYGEDSSYAELFVKGVREVYDYYCRRTVKKLKRRTDAMRLLERPPEFTLPLYN
KTAYVGENVRFGVTITVHPEPRVTWYKSGQKIKPGDDDRKYIFESDKGLYQLTINSVTMDDDAEYTVVARNKYGEDSCKAKLTVTPHPPPSDT
TLRPMFKRLLANAECQEGQSVCFEIRVSGIPPPTLKWEKDGRPLSLGPNIEIIHEGLDYYALHIRDTLPEDTGYYRVTATNTAGSSSCQAHLQ
VERLRYVKQEFKTKEEHERHVQRQIDKTLRMAEILSGTEAVPLTQVAQEALREAAILYKPAVSTKTVKGEYRLETEEKKEERKLRMPYEVPEP
RKYKQTTIEEDQHIKQFVPMSDMKWYKKIRDQFEMPGKIDRVVQKRPKRIRLSRWEQFYVMPLPRITDQYRPKWRIPKLSQDDLEMVRPARRR
TPSPDYYYYYRPRRRSLGDISDEELLLPIDDYLAMKRTEEERLRLEEELELGFSASPPSRSPPRFELSSLRYSSPQAHVKVEEARRDFRYSTY
HIPTKEETSTSYAELRERHARASHRQAHQRQRIMAEREDHELLRPATTTQRLLEYKSELDHLAKEAKSRKKSRRQREVTEITEIEEEYEISKH
AQRETSSSVSRLLRRRRSLSPTYIELMRPVSELIRPRPRPAEEYEDEEEEEEEDVERRSPTPERTRPRSPSPVSSERSLSRFERSARFDIFSR
YESMKAALKTQKTSERKYEVLSQQPFTLDHAPRITLRMRSHRVPCGQNTRFILNVQSKPTAEVKWYHNGVELQESSKIHYTNTSGVLTLEILD
CHIDDSGTYRAVCTNYKGEASDYATLDVTGGDYTTYASQRRDEQVPRSVFPELTRTEAYAVSSFKKTSEMEASSSVREMKSLMTETRESLSSY
EHHASAEMKSAAIEEKSLEEKSTHRKIKTTLAARILTKPRSITVYEGESARFSCDTDGEPVPTVTWLRGGQVISTSARHQVTTSKYKSTFEIS
SVQASDEGNYSVVVENSDGRQEAQFTLTVQKARVTEKAVTSPPRVKSPEPRVKSPEGAKSPKRVKSPEPVTPHPKAVPPTETKPTPTEKVQQK
PVAAPPKIIQSLKAEASKDIAKLTCVVESSALCAKEVTWYKDGRRLKENGHFQFHYSADGTYELKILNLAESDRGEYVCEVSGEGGTSKTNFQ
FVGQAFKSIHEQVSSISETNKKAAPKTAEPTETKKTEPKAPAPISTKPVIVTGLQDTTVSSDSVAKFAVKVTGEPQPTVIWTKDGKAISQGGK
YKLSEDKGAFFLEIHKTDTSDSGLYTCTITNSAGSVSSSCKLTIKVVEDTEIQKVSAQKTSEITSQKKASVQEEISKKALISEEIKTSEVKSH
EKLALKEEASTVLISEKVKKSEAASLEKSVVHEEITKTSQASEEIRTHAEIKAFSTQMSITAGQKVTLKANIAGATDVKWVLNGVELSNSDEY
RYGISGSDQTLTIRQAGHKDEGILTCIGKTSQGIIKCQYDLTLSKELSDAPAFISQPRSQNVNEGQNVLFSCEISGEPSPEIEWFKNNLPISI
SSNISVSRSRNVYSLEIRNASVSDSGKYTIKAKNFHGQCSATASLTVLPLVEEPPREVVLRTSGDTSLQGSFSSQSVQMSASKQEASFSSFSS
SSASSMTEMKFASMSAQSMSSMQESFVEMSSSSFMGKSSMTQLESSTSRMLKAGLRGIPPKIEALPSDISIDEGKVLTVACAFTGEPTPEITW
SRGGRTIHDQEQRGREMENTDDL (SEQ ID NO: 46)
>NP_990445.1 troponin T, slow skeletal muscle [Gallus gallus]
MSEAEEEYEEEQPEEEEEAAAAAEEEEEEEAEASKPHEEPEEERPRPRPVVPQLAPPKIPEGERVDFDDIHRKRMEKDLLELQTL
IDAHFEQRRREENELVALMERIERRRAERNEQLRSRTEKERERQARLAEEKLRKEEEEAKKRAEDDAKKKKVLSNMPHFGGYLAKAEQRRGKR
QTGREMKLRILAERKKPLHIEHMREDELRAKAKELHDWIQQLESEKFDLMEKLRRQKYEINVLYNRISHAQKFKKVVGKGRVGGRWK (SEQ
ID NO: 47)
>tr|A0A1D5PLV5|A0A1D5PLV5_CHICK Uncharacterized protein OS = Gallus gallus OX = 9031
GN = TNNT1 PE = 4 SV = 1
AAEASKPHEEPEEERPRPRPVVPQLAPKIPEGERVDFDDIHRKRMEKDLLELQTLIDAHFEQRRREENELVALMERIERRRAERNEQLRSRTE
KERERQARLAEEKLRKEEEEAKKRAEDDAKKKKVLSNMPHFGGYLAKAEQRRGKRQTGREMKLRILAERKKPLHIEHMREDELRAKAKELHDW
IQQLESEKFDLMEKLRRQKYEINVLYNRISHAQKFKKVVGKGRVGGRWK (SEQ ID NO: 48)
>XP_419242.3 troponin I, slow skeletal muscle [Gallus gallus]
MPEPRERKSKITASRKLLLKSLMLAKAKEEWEQEIVDKQSEKERYLSERITPLHTSGLSLSQLQDLCRELHEKVEIVDEERYDIEAKCNHNTR
EIKDLKLKVLDLRGKFKRPPLRRVRVSADAMLRALLGSKHKVSMDLRANLKSVKKEDTEKERPVEVGDWRKNVEAMSGMEGRKKMFDAAKSPT
GQ (SEQ ID NO: 49)
>tr|F1NUT9|F1NUT9_CHICK Uncharacterized protein OS = Gallus gallus OX = 9031 GN = TNNI1
PE = 4 SV = 3
MLAKAKEEWEQEIVDKQSEKERYLSERITPLHTSGLSLSQLQDLCRELHEKVEIVDEERYDIEAKCNHNTREIKDLKLKVLDLRGKFKRPPLR
RVRVSADAMLRALLGSKHKVSMDLRANLKSVKKEDTEKERPVEVGDWRKNVEAMSGMEGRKKMFDAAKSPTGQ (SEQ ID NO: 50)
>tr|B5DH00|B5DH00_SALSA Fast myotomal muscle troponin-T-1 OS = Salmo salar OX = 8030
GN = TNNT3 PE = 2 SV = 1
MSDTEEVEAQKPQFKVPKIPDGDKVDFDDIQKKRQNKDLIELQALIDAHFEHRKKEEEELIALKERIEKRRAERAEQNRIRSEKEKERAARRE
EERLKREEADAKKKADEDAKKKSALSSMGSNYSSHLQKADSKRGGKKETEREKKKKILAGRRKALNIDHLNEEKLKEKAKELHEWMQTLESEK
FDHIERLKRQKYEVTTLRKRVEELSKFSKKGKTVRRK (SEQ ID NO: 51)
>tr|O57559|O57559_CHICK Troponin T variant TnTx7-e16 OS = Gallus gallus OX = 9031 GN = TNNT3
PE = 2 SV = 1
MSDTEEVEHGEAHEAEEVHEEAHHEEAHHEEAHHEEAHHAEAHHAEAHHEEAHAHAEEVHEPAPPPEEKPRIKLTAPKIPEGEKVDFDDIQKK
RQNKDLIELQALIDSHFEARRKEEEELVALKERIEKRRAERAEQQRIRAEKEKERQARLAEEKARREEEDAKRKAEDDLKKKKALSSMGASYS
SYLAKADQKRGKKQTARETKKKVLAERRKPLNIDHLNEDKLRDKAKELWDWLYQLQTEKYDFAEQIKRKKYEILTLRCRLQELSKESKKAGAK
GKVGGRWK (SEQ ID NO: 52)
>tr|A0A287BD71|A0A287BD71_PIG Myotubularin 1 OS = Sus scrofa OX = 9823 GN = MTM1 PE = 3
SV = 1
MASAPTSKYNSHSLENESIKRTSRDGVNRDMGEAVPRLPGETPITDKEVIYICPFNGPIKGRVYITNYRLYLRSLETDSALILDVPLGVISRI
EKMGGATSRGENSYGLDITCKDMRNLRFALKQEGHSRRDMFEILTRYAFPLAHSLPMFAFLNEEKFNVDGWTVYNPVEEYRRQGLPNHHWRIT
FINKCYELCDTYPALLVVPYRAADEDLRRVATFRSRNRIPVLSWIHPENKTVIVRCSQPLVGMSGKRNKDDEKYLDVIRETNRQVNKLTIYDA
RPNVNAVANKATGGGYESDDAYHNAELFFLDIHNIHVMRESLKKVKDIVYPNVEESHWLSSLESTHWLEHIKLVLTGAIQVADRVSSGKSSVV
VHCSDGWDRTAQLTSLAMLMLDSFYRSIEGFEILVQKEWISFGHKFASRIGHGDKNHADADRSPIFLQFIDCVWQMSKQFPTAFEFNEHFLIT
ILDHLYSCRFGTFLYNCESARERQKVTERTVSLWSLINSNKDKFKNPFYTKEINRVLYPVASMRHLELWVNYYIRWNPRIKQQQPNPVEQRYM
ELLALRDEYIKRLEELQLANSAKLSEPAAAPSSPSQMVSHVQTHF (SEQ ID NO: 53)
>sp|Q4PS85|MYOZ1_PIG Myozenin-1 OS = Sus scrofa OX = 9823 GN = MYOZ1 PE = 2 SV = 1
MPLSGTPAPNKKRKSSKLIMELTGGGQESSGLNLGKKISVPRDVMLEELSLLTNRGSKMFKLRQMRVEKFIYENHPDVFSDSSMDHFQKFLPT
VGGQLGTAGQGFSYSKGSSGGQAGGSSSAGQYGSGQQHHHQGSGSGSGGAGGPGSQTGRGGDAGTTGVGETGTGDQAGGEGKHITVFKTYISP
WEKAMGVDPHQKVELGIDLLAYGAKAELPQYKSFNRTAMPYGGYEKASKRMTFQMPKFDLGPLLSEPLVLYNQNLSNRPSFNRTPIPWLSSGE
PVDYNVDIGIPLDGETEEL (SEQ ID NO: 54)
>sp|P19352|TPM2_CHICK Tropomyosin beta chain OS = Gallus gallus OX = 9031 GN = TPM2 PE = 1
SV = 1
MEAIKKKMQMLKLDKENAIDRAEQAEADKKQAEDRCKQLEEEQQGLQKKLKGTEDEVEKYSESVKEAQEKLEQAEKKATDAEAEVASLNRRIQ
LVEEELDRAQERLATALQKLEEAEKAADESERGMKVIENRAMKDEEKMELQEMQLKEAKHIAEEADRKYEEVARKLVVLEGELERSEERAEVA
ESKCGDLEEELKIVTNNLKSLEAQADKYSTKEDKYEEEIKLLGEKLKEAETRAEFAERSVAKLEKTIDDLEDEVYAQKMKYKAISEELDNALN
DITSL (SEQ ID NO: 55)
>sp|Q3ZC09|ENOB_BOVIN Beta-enolase OS = Bos taurus OX = 9913 GN = ENO3 PE = 2 SV = 1
MAMQKIFAREILDSRGNPTVEVDLHTAKGRFRAAVPSGASTGIYEALELRDGDKSRYLGKGVLKAVEHINKTLGPALLEKKLSVVDQEKVDKF
MIELDGTENKSKFGANAILGVSLAVCKAGAAEKGVPLYRHIADLAGNPELILPVPAFNVINGGSHAGNKLAMQEFMILPVGASSFREAMRIGA
EVYHHLKGVIKAKYGKDATNVGDEGGFAPNILENNEALELLKTAIQAAGYPDKVVIGMDVAASEFYRNGKYDLDFKSPDDPARHISGEKLGEL
YKNFIKNYPVVSIEDPFDQDDWATWTSFLSGVNIQIVGDDLTVTNPKRIAQAVEKKACNCLLLKVNQIGSVTESIQACKLAQSNGWGVMVSHR
SGETEDTFIADLVVGLCTGQIKTGAPCRSERLAKYNQLMRIEEALGDKAVFAGRKFRNPKAK (SEQ ID NO: 56)
>sp|A6QLT4|MTM1_BOVIN Myotubularin OS = Bos taurus OX = 9913 GN = MTM1 PE = 2 SV = 2
MASAPTSKYNSHSLENESIKRTSRDGVNRDVGETLPRLPGEIRITDKEVIYICPFNGPIKGRVYITNYRLYLRSLETDSALILDVPLGVISRI
EKMGGATSRGENSYGLDITCKDLRNLRFALKQEGHSRRDMFEILTRYAFPLAHSLPIFAFLNEEKFNVDGWTVYNPVEEYRRQGLPNHHWRIT
FINKCYKLCDTYPALLVVPYRASDEDLRRVATFRSRNRIPVLSWIHPENKTVIVRCSQPLVGMSGKRNKEDERYLDVIRETNRQVNKLTIYDA
RPNVNAVANKATGGGYESDDVYHNAELFFLDIHNIHVMRESLKKVKDIVYPNVEESHWLSSLESTHWLEHIKLVLTGAIQVADRVSSGKSSVV
VHCSDGWDRTAQLTSLAMLMLDSFYRSIEGFEILVQKEWISFGHKFASRIGHGDKNHADADRSPIFLQFIDCVWQMSKQFPTAFEFNERFLIT
ILDHLYSCRFGTFLYNCESAREKQKVTERTVSLWSLINSNKDKFKNPFYTKEINRVLYPVASMRHLELWVNYYIRWNPRIKQQQPNPVEQRYM
ELLALRDEYIKRLDELQLANSAKLSDPSASPSSPSQMMPHVQTHF (SEQ ID NO: 57)
>sp|Q7YS81|MYOG_BOVIN Myogenin OS = Bos taurus OX = 9913 GN = MYOG PE = 2 SV = 2
MELYETSPYFYQEPHFYDGENYLPVHLQGFEPPGYERAELSLSPEARVPLEDKGLGPAEHCPGQCLPWACKVCKRKSVSVDRRRAATLREKRR
LKKVNEAFEALKRSTLLNPNQRLPKVEILRSAIQYIERLQALLSSLNQEERDLRYRGGGGPQAAVPSECSSHSASCSPQWGSALEFGPNPGDH
LLPADPTDAHNLHSLTSIVDSITVEDVAAAFPDETIPN (SEQ ID NO: 58)
>sp|P49812|MYOG_PIG Myogenin OS = Sus scrofa OX = 9823 GN = MYOG PE = 3 SV = 1
MELYETSPYFYQEPHFYDGENYLPVHLQGFEPPGYERTELSLSPEARVPLEDKGLGTPEHCPGQCLPWACKVCKRKSVSVDRRRAATLREKRR
LKKVNEAFEALKRSTLLNPNQRLPKVEILRSAIQYIERLQALLSSLNQEERDLRYRGGGGPQPGVPSECSSHSASCSPEWGSALEFGPNPGDH
LLTADPTDAHNLHSLTSIVDSITVEDVAVAFPDETMPN (SEQ ID NO: 59)
>sp|P31696-5|AGRIN_CHICK Isoform 5 of Agrin OS = Gallus gallus OX = 9031 GN = AGRN
MGGSGAAATLALGLALGLALGGWANCPERELQRREEEANVVLIGTVEEIMNVDPVHHTYSCKVRVWRYLKGKDIVTHEILLDGGN
KVVIGGFGDPLICDNQVSTGDTRIFFVNPAPQYMWPAHRNELMLNSSLMRITLRNLEEVEHCVEEHRKLLADKPNSYFTQTPPTPRDACRGML
CGFGAVCERSPTDPSQASCVCKKTACPVVVAPVCGSDYSTYSNECELEKAQCNQQRRIKVISKGPCGSKDPCAEVTCSFGSTCVRSADGQTAG
CVCPASCSGVAESIVCGSDGKDYRSECDLNKHACDKQENVFKKFDGACDPCKGILNDMNRVCRVNPRTRRVELLSRPENCPSKREPVCGDDGV
TYASECVMGRTGAIRGLEIQKVRSGQCQHQDKCKDECKFNAVCLKRWHARCSCDRITCDGTYRPVCARDSRTYSNDCERQKAECHQKAAIPVK
HSGPCDLGTPSPCLSVECTFGATCVVKNREPVCECQQVCQGRYDPVCGSDNRTYGNPCELNAMACVLKREIRVKHKGPCDRCGKCQFGAICEA
ETGRCVCPTECVPSSQPVCGTDGNTYGSECELHVRACTQQKNILVAAQGDCKSCGTTVCSFGSTCVGGQCVCPRCEQQPLAQVCGTDGLTYDN
RCELRAASCQQQKSIEVAKMGPCEDECGSGGSGSGDGSECEQDRCRHYGGWWDEDAEDDRCVCDFTCLAVPRSPVCGSDDVTYANECELKKTR
CEKRQNLYVTSQGACRALTTTPPPLPVVHCSQTIYGCCPDNMTLALGVGAAGCPSTCQCNPYGSYGGTCDPATGQCSCKPGVGGLKCDRCEPG
FWNFRGIVTDSKSGCTPCNCDPVGSVRDDCEQMTGLCSCKTGITGMKCNQCPNGSKMGMAGCEKDPSAPKSCEEMSCEFGATCVEVNGFAHCE
CPSPLCSEANMTKVCGSDGVTYGDQCQLKTIACRQGQLITVKHVGQCHESITHTSHTMPPTPLPTLPLDKLIVPPPLQLTTQAPEPTELATTS
LLMEASPTTRSHPTTRRVTTTRPVTTPWMTHGVLKTTVRPLSTSPVVLATTQPPYAESGSAEGSGDQEMSISGDQESSGAGSAGEEEVEESQV
TPTPAIERATCYNTPLGCCSDGKTAAADAEGSNCPATKVFQGVLILEEVEGQELFYTPEMADPKSELFGETARSIESALDELFRNSDVKNDFK
SIRVRDLGQSSAVRVIVESHFDPATSYTAADVQAASLKQIRASKKRTILVKKPQQEHVKFMDEDWIPRIFTTTITTTTATTMAPATTRRHTTA
SAATTAHILRQDTVGHPSAKLAAPASTRRPTSTLPTTARRKPTRQPPSTTKKPSRPCDSHPCLHGGTCEDDGREFTCRCPAGKGGAVCEKPIR
YFIPSFGGKSYLAFKMMKAYHTVRIAMEFRATELSGLLLYNGQNRGKDFISLALVGGFVELRENTGSGTGVITSKVRVEPGKWHQLVVNRNRR
SGMLAVDGEHVSGESPTGTDGLNLDTDLFVGGAPEDQMAVVAERTAATVGLKGSIRLLDVNNQMYDLREKGSDVLYGSGVGECGNDPCHPNPC
HHGASCHVKEAEMFHCECLHSYTGPTCADERNPCDPTPCHISATCLVLPEGGAMCACPMGREGEFCERVTEQDHTMPFLPEFNGFSYLELNGL
QTLFLTCRQMSMEVVFLAKSPSGMIFYNGQKTDGKGDFVSLALHDGYLEYRYDLGKGAAVLRSKEPVPLNTWISVLLERSGRKGVMRINNGER
VMGESPVPHAFLNLKEPFYVGGAPDFSKLARAAAISTSFYGAVQRISIKGVPLLKEQHIRSAVEISTFRAHPCTQKPNPCQNGGTCSPRLESY
ECACQRGFSGAHCEKVIIEKAAGDAEAIAFDGRTYMEYHNAVTKSEKALQSNHFELSIKTEATQGLILWSGKGLERSDYIALAIVDGFVQMMY
DLGSKPVVLRSTVPINTNHWTHIKAYRVQREGSLQVGNEAPITGSSPLGATQLDTDGALWLGGMERLSVAHKLPKAYSTGFIGCIRDVIVDRQ
ELHLVEDALNNPTILHCSAK (SEQ ID NO: 60)
>sp|P21566|COF2_CHICK Cofilin-2 OS = Gallus gallus OX = 9031 GN = CFL2 PE = 1 SV = 2
MASGVTVNDEVIKVFNDMKVRKSSTPEEIKKRKKAVLFCLSDDKKQIIVEEAKQILVGDIGDTVEDPYTAFVKLLPLNDCRYALYDATYETKE
SKKEDLVFIFWAPESAPLKSKMIYASSKDAIKKKFTGIKHEWQVNGLDDIKDRSTLGEKLGGNVVVSLEGKPL (SEQ ID NO: 61)
>sp|Q679P3|PDLI7_CHICK PDZ and LIM domain protein 7 OS = Gallus gallus OX = 9031 GN = PDLIM7
PE = 1 SV = 1
MESYKVMLNGPAPWGFRLQGGKDFSMPLSISRLTPGGKAAQAGVGVGDWVLYIDGESTGTMTHIEAQNRIRACGDRLCLTLSRAQNHLGKPQK
DSLPCSEPPKYNFAPSTALNKTARPFGASSPPNPRPGLVTKPVTYVPLAPACTPQHNGQVSVPDPSPGAAMKTEPGLAPRTPAATPGPTSRPP
WAVDPSFAERYAPDKTSTVLSKHSQPATPTPMQNRSSIVQAAQQAPESPGRTPLCYKCNKIIRGRYLVALGHYYHPEEFTCCQCRKVLDEGGF
FEEKGSIFCPKCYDTRYAPSCAKCKKKITGEVMHALKMTWHVQCFTCAACKTPIRNRAFYMEEGQPYCERDYEKMFGTKCRGCDFKIDAGDRF
LEALGFSWHDTCFVCAICQTNLEGKTFYSKKDKPLCKSHAFSHV (SEQ ID NO: 62)
>tr|F1NQD9|F1NQD9_CHICK Radixin OS = Gallus gallus OX = 9031 GN = RDX PE = 4 SV = 3
MPKPINVRVTTMDAELEFAIQPNTTGKQLFDQVVKTVGLREVWFFGLQYVDSKGYSTWLKLNKKVTQQDVRKENPLQFKFRAKFFPEDVSEEL
IQEITQRLFFLQVKEAILNDEIYCPPETAVLLASYAVQSKYGDYNKEIHKLGYLANDRLLPQRVLEQHKLTKEQWEERIQNWHEEHRGMLRED
SMMEYLKIAQDLEMYGVNYFEIKNKKGTELWLGVDALGLNIYEHDDKLTPKIGFPWSEIRNISFNDKKFVIKPIDKKAPDFVFYAPRLRINKR
ILALCMGNHELYMRRRKPDTIEVQQMKAQAREEKHQKQLERAQLENEKKKREIAEKEKERIEREKEELMERLRQIEEQTMKAQKELEEQTRRA
LELDQERKRAKEEAERLEKERRAAEEAKAALAKQAADQMKNQEQLAAELAEFTAKIALLEEAKKKKEEEASEWQHKAFAAQEDLEKTKEELKS
VMSAPPPPPPPPVIPPTENEHDEHDENNAEASAELSSDGVMNHRSEEERVTETQKNERVKKQLQALSSELAQARDETKKTQNDVLHAENVKAG
RDKYKTLRQIRQGNTKQRIDEFEAM (SEQ ID NO: 63)
>XP_025008315.1 nebulin isoform X22 [Gallus gallus]
MEEEEYEEVVEYYIEETIVEEGEPYEVVTEITDSTSTEFTGPTTITRTIEYEKTSGEGAATPVRKKTIRTKMDTSKFLTPYLQHSNKMKDLES
ENKYKEKFNKERGKPYASTIDTPEIRRIKKVQEQLSEVKYRMAGEAARTICHVDEKAWDIEHAKKVSQQVSKVLYKQNWEENKDKYLLPPDAP
ELVNAIKNTAMFSKKLYTEDWEGDKTLFYPYNDSPELRRVAQAQKALSDIVYKKGHDERKSKYTSLPDPPDVEQAKKVTRQLSDIIYHDDYKN
KIKGKWSQTPCYDVVIAKMNAENLSMKKYQEDFENVKDQIYFMQTETPEYEANKRVSDNVSKIKYRADYEKNKAIADYNVLPATENPLLRQLK
TAGDVLSDKLYKEAYERSKGTSMNYCETPKFQTDNALKNFSDVKYKDAYQKNILGHYLGSFEDPHQIHCMKVEAMKSDKNYKADYEEEKTKCY
FPQTITQEYEAIKKLEQCKDHTYKKHPDQIKFTPVTDSPVQKQAEINSKQLSDKLYRSSGEEVKHKYTLPPDVPQFIQARYNAANVSDAYYKQ
DYHDLIAKGNNVSLDAIPITRAKASRNIASDYKYKEAYEKAKGQQVGFKSLQDDPKLVHYMHVAKIQSDREYKKDYEKSKTNYHTPPDTFSIQ
AAKKSQDVASTAHYKNLIHHYTYLPDAMDVELAKNMMQIQSDNVYKQDYNSWFKGIGWSPLGSLDVEKAKKAGDALNEKKYRQHPDTIKFTSV
PDSMTMVLAQHNTKQLSDVAYKQEGEKVKHKYKLDPDVPQFIQARVNAFNLSDANYKADWKKTIAKGYDLKPDAIPIIAAKASRNIASDYKYK
ESYEKDKGRQVGYRSLQDDPKLVHYMHVAKMQSDREYKKDYEVTKTKYHTPLDMFSVTAAKKAQEAVTNTGYKQLIHHYTLLPDSVNLELSRN
MMQLQSDNMYKADFNNWLRGVGWLPIQSLEVEKAKKASEILSEKKYRQHPDKLKYSIPLDAMEQVLAKQNAKTMNKRLYTDKWNKEKTSIHVM
PDTPEILQSRVNQITMSNKLYKAGWEEDKKKGYDMRPDAIPIKAAKTSQDIASDYKYKLAHEKAKGKHIGFRSLEDDPKLVHFMQVAKMQSDR
EYKKDYEKAKTNFHTPVDMLSVVAAKKAQEVATNANYKNLIHVYNVLPDAMSLELAKNMMQIQSNNQYRAEYDESMKGVGWMPLGSLEAEKNK
KAMEILSEKKYRQHPDKLKYSVPVDSMNMALALHNAKIMDEHQYKQAWEEDKKKVHMTPDIPQFALAKANAFNISDKMYRHSFEEARKKGYDL
RSDAIPIKAAKASRDIASDYKYKLGYEQDKGKLVGFRSLQDDPKLVHYMQVAKMQSDREYKKAYETSKTHYQTPSDALSIMAAKEAQDRVTNA
NYKRLIHHYMLLPDAMSFELYRNMNQIQSNNEYKQDYNEWFKGIGWSPAGSLDVEKSKKATEIASDQKYRQHPSIFPFTKQIDAMDMVLAKHN
ADIMNKHAYTQAWEKDKTQVHVMPDTPDILQAKQNKANYSQKQYKLDWQEMIKKGYDLTPEAISVKAAKASRDIASDYKYKEGYRKQQGHHIG
FRSLQDDPKMMWSMQVAKMQSEREYKKDFEKWKTKFNMPVDMLGFLLAKKCQELVSDIDYKHMLHRWTCLPDQNDVTQAKRVYELQSDNLYKS
DLQWLRGIGWSPLGSLESEKNKKASEILSEKKYRQHPDTIKFTSIPDAMNIILAKSNAKNRSDILYREAWDKDKTQVHIMPDTPEILLAKSNL
INTSDKHYKLGYEELRRKGYDLPPDAIPLKSAKASRDIASEYQYKTAYRKQLGHHVGARNIEDDPKMMWSMHVAKIQSDREYKKAFEKTKTHF
SSPVDMLGIVLAKKCQELVSDVDYKHLLHRWTCLPDQNDVVQARKVYDLQSDNVYKSDLQWLRGIGWSPLGSLDEEKNKRASMILSDKKYRQH
PDTIKFTSLPDSMPMVLAKHNSEIMNHRSYIAAWEKDKTSIHIMPDTPGILLAQQNKVNYSEKMYRLAMEEDKKKGYDLRADAIPIKAAKASR
DIASDYKYKEGYRKQLGHHIGARNIEDDPKMMWSMHVAKVQSDREYKKAFEKTKTHFSSPVDMLGIVLAKKCQELVSDVDYKHLLHRWTCLPD
QNDVVQARKVYDLQSDNVYKSDLQWLRGIGWSPLGSLDEEKNKRASMILSDKKYRQHPDTIKFTSLPDSMPMVLAKHNSEIMNHRSYIAAWEK
DKTSIHIMPDTPGILLAQQNKVNYSEKMYRLAMEEDKKKGYDLRADAIPIKAAKASRDIASDYKYKEGYRKQLGHHIGARNIEDDPKMMWSMH
VAKVQSDREYKKAFEKTKTHFSSPVDMLGIVLAKKCQELVSDVDYKHLLHRWTCLPDQNDVVQARKVYDLQSDNVYKSDLQWLRGIGWSPLGS
LDEEKNKRASMILSDKKYRQHPDTIKFTSLPDSMPMVLAKHNSEIMNHRSYIAAWEKDKTSIHIMPDTPGILLAQQNKVNYSEKMYRLAMEED
KKKGYDLRADAIPIKAAKASRDIASDYKYKEGYRKQLGHHIGARNIEDDPKMMWSMHVAKVQSDREYKKAFEKTKTHFSSPVDMLGIVLAKKC
QELVSDVDYKHLLHRWTCLPDQNDVVQARKVYDLQSDNVYKSDLQWLRGIGWSPLGSLDEEKNKRASMILSDKKYRQHPDTIKFTSLPDSMPM
VLAKHNSEIMNHRSYIAAWEKDKTSIHIMPDTPGILLAQQNKVNYSEKMYRLAMEEDKKKGYDLRADAIPIKAAKASRDIASDYKYKEGYRKQ
LGHHIGARNIEDDPKMMWSMHVAKIQSDREYKKAFEKTKTHFSSPVDMLGIVLAKKCQELVSDVDYRHYLHQWICLPDQNDVIHARKAYDLQS
DNFYKSDLEWMRGIGWVPIGSLDIEKAKRAGQILSDKVYRQPPDTIKFTSVTDSLEMTLAKHNAEMMNKRLYTEAWDKDKTQIHIMPDTPEIT
LAKQNMHNYSEKLYKQAMEEAKKKGYDLRSDAIPIQAAKASRQIASDYKYKEGYRKQLGHHIGARNIEDDPKMMWSMHVAKIQSDREYKKAFE
KTKTHFSSPVDMLGIVLAKKCQELVSDVDYRHYLHQWICLPDQNDVIHARKAYDLQSDNFYKSDLEWMRGIGWVPIGSLDVEKAKRAGQILSD
KVYRQPPDTIKFTSVTDSLEMTLAKHNAETMNKRLYTEAWNKDKTTIHVMPDTPEILLAKQNQAHYSQKMYKLALEESKKKGHDLRFDAIPIQ
AAKASREIASDYKYKEGYRKQLGHHIGARNIEDDPKMMWSMHVAKIQSDREYKKAFEKTKTHFSSPVDMLGIVLAKKCQELVSDVDYRHYLHQ
WICLPDQNDVIHARKAYDLQSDAVYKSDLEWLKGIGWVPIGSLDVEKAKKAGEILSDRKYRQPADQIKFTSVTDSLAMMLAKHNAEIMNKRLY
TEAWDADKTSIHVMPDTPTILLAKANAANVSHKHYVQAWEDAKKKGYDMRADAIPIRSAKASRDIASDYKYKEAHEKQKGHYIGCRTAKEDPK
LSWAARAMLLQNDRIYRKAYNDSKAHIHMPVDAMSLQAAKECQTLVSDVDYRHYLHQWTCLPDQNDVMHARKAYDLQSDNVYKSDLEWLRGIG
WLTEGSVDVIKAKKAQEILSDRLYRTQPDKMKFTSITDTPDVVQAKINAMQLSNHLYREVWDKDKTQISIPSDTPELLQSKLNALNISNKHYQ
KAWDEAKAKNYDLRADAIPIKHAKASRDIASEYKYKEAHEKQKGHYIGCRTAKEDPKLSWAARAMLLQNDRIYRKAYNDSKAHIHMPVDAMSL
QAAKECQTLVSDVDYRHYLHQWTCLPDQNDVMHARKAYDLQSDNVYKSDLEWLRGIGWLTEGSVDVVKAKKAQEILSDRLYRTQPDKMKFTSV
TDAPDVVQAKINAMQLSNRLYREAWDKDKTQISIPSDTPEMLQSKVNALNISNKHYQKAWDEAKAKNYDLRADAIPIKHAKASRDIASEYKYK
EAHEKQKGHYIGCRTAKEDPKLSWAARAMLLQNDRIYRKAYNDSKAHIHMPVDAMSLQAAKECQTLVSDVDYRHYLHQWTCLPDHNDVVHARK
AYDLQSDAVYKSDLEWLRGIGWLPNDSPGVQRVKHAQDLLSDKVYRTPIDSVKYTSVVDSPDILLAKMNAEQLSIPKYKEAWEKDKTMIHIMP
DTPEITLARSNAHNYSQKLYKEAWDEVKMSYDLRADAIPIKAAKASREIASDYKYKLDHEKQKGHYVGVPNAKADTKIRFALGIGKVQSELEY
KKHFAKWKTQCHLPVDMLSIQSAKHGQSLVSDVDYRHYLHQWICLPDQNDVIHARKAYDLQSDAVYKSDLEWLRGIGWLPNDSLGINHVKHAG
DLLNERKYRTKAETLHFTPVADRVDYVTAKKSGEILSDIKYHKDWNEVKSNYTLTDTPQLDMAREAARILNQSLYKESWEKEKATGYLLPPDT
VQIRHAKHSNDVQSELKYKADYVKQRGHYVGVASMRDDPKLVWFEHAGEIQNDRLYKSNYHKTKSKIHIPADIMSVVAAKECQALVSDVDYRH
YLHQWTCHPDQNDCIQARKAYDLQSDNIYKSDLEWLRGCGWIPLGSVEHKKVKHAQELINKRAYTKDAIENFSKYTSVVDTPDIVLAKINSVN
QSDLKYKETFNLEKGQYIGSDDTPELNHARDMSLLYSDKLYKRDWEVCKPIGYTLDAKYIPLVGAKHANYVNSELKYKEIYEKLKGHYLAGKD
IGDFPSVVHSLAFQKIRSALAYRKNYEDTKTRVHIPSDMMNHVLAKKCQYILSDLEYRTYLHHWNCSPEEHDVIQARRAQEILSDVVYKDDLN
WLKGIGCYVWDTPQILHAKKSYDLQSQIKYTAAGKENFQNFGVVTDTPVYVTAVQSGINASDVKYKEDYHKTKDKYTTVTETADSERVQNLKH
LFSNNLYKEAWDRVKATSYIMPSDAVSLARAKELKHNASIVKYREEYDKFKALYTLPRSVEDDPNTARCLRVGKLNIDRLYKETYEKNKAKVH
IIPDMVDIIAAKDAQKKISEIDYRTHLHDWICLPDLQINAHVRKVADQISDVVYKDDLNWLKGIGCFVWDTPEILHAKHAYDLRSDIKYKSDA
DKMKSKYTVVMDTPVYVQNILSGLNASEVIYRGDYLKKVRGKMIPTDKTVDLQRAHHANKIQSENLYRWAGLKALPTGYSLPKDTPGFQHAKH
VQHIGSDLKYKEAYEHMKAKGYTLGPNDVGFENVKKVNQVINERLYRATYHKNKDKIHTTPDTPEIRQVRATQEAVSDLIYKSDFFKLQGHLI
SLPFTPQVLHCRYVGDITSDNKYKEDLKWLQGLGCFLYDTPDMVRARQLRKLWSNYVYTDSAKKMRDKYSVVLDTPGYRTVQELKTHLSDLVY
RAAGKELKTKYTSVLNTPDFLRAKEGQRIQSQYLYVALATKERPHHHAGNQTPAFTHARHVKDMVSETKYKIQYEKMKDKYTPVLDTPILIRA
KRAYLNASDLRYKETFENTKGKYHTVKDALDIVYHRKVTDDISSVKYKENYMSQLGIWRSIPDRPEHFHHRAVTDAISDVKYKEDLSWLRGIG
CYAWDTPDFALAEKNKVLYSGHKYKETFEKTKSHFKYVADSPINRHFKHATQLLDANSYKSLAKMLLKQGCNEILRPDILTALYNSYLWSQVE
YKKDYEKKKDKYTTVVDTPENIRTAKVNKQISDIIYKLEYNKAKPKGYTTIHDTPMLLHVRKVKDRISDLKYKELYERNKSHCNVVADSVHIK
TPRHAYKLNSNLDYKKKYEAAKAHWHWIADRPDFVQAAKSSLQQSDYEYKLDREYLKGCKLSVTDDKNTVLALNNAILASDIKYKEKHNKARG
TCLVVPDTPQILLAKNVSSLVSENKYKEHSKKQLPRGSYTTLPETRDTAHVKEVTKNVSDTNYKKKFVKEKGKSNYSIMLEPPEVKHAMEVAK
KQSTVEYKKDAKSKLHYTPIADRPDIKKATQAAKLISDIEYKKRGEAGLGVTMLGRPDIELAKEVSKLTSQVKYKENFSKEKGKKPKYDLKEA
KIYKTMKDAHNIASEVKYKADLKKLHKPVTDMSESLIMNHVLNTSQLASAVKYKEKYEKEKGKPMLDFETPTYLTAKESQLMQSEKEYRKDLE
EGVKGKGLSVLEETPDMLRAKNATQILNEKEYKKALELEIKGKGLSELALETPDFVRAKNATDIASQIKYKQLAEMEKANYTSVVDTPEIIHA
QQVKNLSSQKKYKEEAEKTMPYYVPVADTPEMQRVRENQKNFSTLQYQWDLQNSKGKVTVVQDTPEMLRVKENQKNFSSIKYKESIGKGTPIP
DLPEVKRVKETQKHISSVLYKEHLAKGTPTPMTPEMERAKRNQENISSVLYSDSFRKQVQGKAAYVLDTPEMRRVRETQKHISTVKYHEDFEK
NKGTFTPVVTDPITERVKKNMHDFSDISYRGIQRRVVEMEQKRVDQDQENLTGLRVWRTNPGSVFDYDPAEDNIQSRSLHMISVQAQRRSREH
SRSASALSISGGDEKSEPSEGVNQHLSYYSSGGFFTTTATVGYKHAKTIELPQQRSASVATQQTTVSSVPSHPSTAGKTYRAMYDYTAADADE
VSFKDGDTIVNVQAIDEGWMYGTVQRTGKTGMLPANYVEAV (SEQ ID NO: 64)
>XP_025008310.1 nebulin isoform X17 [Gallus gallus]
MEEEEYEEVVEYYIEETIVEEGEPYEVVTEITDSTSTEFTGPTTITRTIEYEKTSGEGAATPVRKKTIRTKMDTSKELTPYLQHS
NKMKDLFSENKYKEKFNKERGKPYASTIDTPEIRRIKKVQEQLSEVKYRMAGEAARTICHVDEKAWDIEHAKKVSQQVSKVLYKQNWEENKDK
YLLPPDAPELVNAIKNTAMFSKKLYTEDWEGDKTLFYPYNDSPELRRVAQAQKALSDIVYKKGHDERKSKYTSLPDPPDVEQAKKVTRQLSDI
IYHDDYKNKIKGKWSQTPCYDVVIAKMNAENLSMKKYQEDFENVKDQIYFMQTETPEYEANKRVSDNVSKIKYRADYEKNKAIADYNVLPATE
NPLLRQLKTAGDVLSDKLYKEAYERSKGTSMNYCETPKFQTDNALKNFSDVKYKDAYQKNILGHYLGSFEDPHQIHCMKVEAMKSDKNYKADY
EEEKTKCYFPQTITQEYEAIKKLEQCKDHTYKKHPDQIKFTPVTDSPVQKQAEINSKQLSDKLYRSSGEEVKHKYTLPPDVPQFIQARYNAAN
VSDAYYKQDYHDLIAKGNNVSLDAIPITRAKASRNIASDYKYKEAYEKAKGQQVGFKSLQDDPKLVHYMHVAKIQSDREYKKDYEKSKTNYHT
PPDTFSIQAAKKSQDVASTAHYKNLIHHYTYLPDAMDVELAKNMMQIQSDNVYKQDYNSWFKGIGWSPLGSLDVEKAKKAGDALNEKKYRQHP
DTIKFTSVPDSMTMVLAQHNTKQLSDVAYKQEGEKVKHKYKLDPDVPQFIQARVNAFNLSDANYKADWKKTIAKGYDLKPDAIPIIAAKASRN
IASDYKYKESYEKDKGRQVGYRSLQDDPKLVHYMHVAKMQSDREYKKDYEVTKTKYHTPLDMFSVTAAKKAQEAVTNTGYKQLIHHYTLLPDS
VNLELSRNMMQLQSDNMYKADFNNWLRGVGWLPIQSLEVEKAKKASEILSEKKYRQHPDKLKYSIPLDAMEQVLAKQNAKTMNKRLYTDKWNK
EKTSIHVMPDTPEILQSRVNQITMSNKLYKAGWEEDKKKGYDMRPDAIPIKAAKTSQDIASDYKYKLAHEKAKGKHIGFRSLEDDPKLVHFMQ
VAKMQSDREYKKDYEKAKTNFHTPVDMLSVVAAKKAQEVATNANYKNLIHVYNVLPDAMSLELAKNMMQIQSNNQYRAEYDESMKGVGWMPLG
SLEAEKNKKAMEILSEKKYRQHPDKLKYSVPVDSMNMALALHNAKIMDEHQYKQAWEEDKKKVHMTPDIPQFALAKANAFNISDKMYRHSFEE
ARKKGYDLRSDAIPIKAAKASRDIASDYKYKLGYEQDKGKLVGFRSLQDDPKLVHYMQVAKMQSDREYKKAYETSKTHYQTPSDALSIMAAKE
AQDRVTNANYKRLIHHYMLLPDAMSFELYRNMNQIQSNNEYKQDYNEWFKGIGWSPAGSLDVEKSKKATEIASDQKYRQHPSIFPFTKQIDAM
DMVLAKHNADIMNKHAYTQAWEKDKTQVHVMPDTPDILQAKQNKANYSQKQYKLDWQEMIKKGYDLTPEAISVKAAKASRDIASDYKYKEGYR
KQQGHHIGFRSLQDDPKMMWSMQVAKMQSEREYKKDFEKWKTKFNMPVDMLGFLLAKKCQELVSDIDYKHMLHRWTCLPDQNDVTQAKRVYEL
QSDNLYKSDLQWLRGIGWSPLGSLESEKNKKASEILSEKKYRQHPDTIKFTSIPDAMNIILAKSNAKNRSDILYREAWDKDKTQVHIMPDTPE
ILLAKSNLINTSDKHYKLGYEELRRKGYDLPPDAIPLKSAKASRDIASEYQYKTAYRKQLGHHVGARNIEDDPKMMWSMHVAKIQSDREYKKA
FEKTKTHFSSPVDMLGIVLAKKCQELVSDVDYKHLLHRWTCLPDQNDVVQARKVYDLQSDNVYKSDLQWLRGIGWSPLGSLDEEKNKRASMIL
SDKKYRQHPDTIKFTSLPDSMPMVLAKHNSEIMNHRSYIAAWEKDKTSIHIMPDTPGILLAQQNKVNYSEKMYRLAMEEDKKKGYDLRADAIP
IKAAKASRDIASDYKYKEGYRKQLGHHIGARNIEDDPKMMWSMHVAKVQSDREYKKAFEKTKTHFSSPVDMLGIVLAKKCQELVSDVDYKHLL
HRWTCLPDQNDVVQARKVYDLQSDNVYKSDLQWLRGIGWSPLGSLDEEKNKRASMILSDKKYRQHPDTIKFTSLPDSMPMVLAKHNSEIMNHR
SYIAAWEKDKTSIHIMPDTPGILLAQQNKVNYSEKMYRLAMEEDKKKGYDLRADAIPIKAAKASRDIASDYKYKEGYRKQLGHHIGARNIEDD
PKMMWSMHVAKVQSDREYKKAFEKTKTHFSSPVDMLGIVLAKKCQELVSDVDYKHLLHRWTCLPDQNDVVQARKVYDLQSDNVYKSDLQWLRG
IGWSPLGSLDEEKNKRASMILSDKKYRQHPDTIKFTSLPDSMPMVLAKHNSEIMNHRSYIAAWEKDKTSIHIMPDTPGILLAQQNKVNYSEKM
YRLAMEEDKKKGYDLRADAIPIKAAKASRDIASDYKYKEGYRKQLGHHIGARNIEDDPKMMWSMHVAKVQSDREYKKAFEKTKTHFSSPVDML
GIVLAKKCQELVSDVDYKHLLHRWTCLPDQNDVVQARKVYDLQSDNVYKSDLQWLRGIGWSPLGSLDEEKNKRASMILSDKKYRQHPDTIKFT
SLPDSMPMVLAKHNSEIMNHRSYIAAWEKDKTSIHIMPDTPGILLAQQNKVNYSEKMYRLAMEEDKKKGYDLRADAIPIKAAKASRDIASDYK
YKEGYRKQLGHHIGARNIEDDPKMMWSMHVAKIQSDREYKKAFEKTKTHFSSPVDMLGIVLAKKCQELVSDVDYRHYLHQWICLPDQNDVIHA
RKAYDLQSDNFYKSDLEWMRGIGWVPIGSLDVEKAKRAGQILSDKVYRQPPDTIKFTSVTDSLEMTLAKHNAETMNKRLYTEAWNKDKTTIHV
MPDTPEILLAKQNQAHYSQKMYKLALEESKKKGHDLRFDAIPIQAAKASREIASDYKYKEGYRKQLGHHIGARNIEDDPKMMWSMHVAKIQSD
REYKKAFEKTKTHFSSPVDMLGIVLAKKCQELVSDVDYRHYLHQWICLPDQNDVIHARKAYDLQSDAVYKSDLEWLKGIGWVPIGSLDVEKAK
KAGEILSDRKYRQPADQIKFTSVTDSLAMMLAKHNAEIMNKRLYTEAWDADKTSIHVMPDTPTILLAKANAANVSHKHYVQAWEDAKKKGYDM
RADAIPIRSAKASRDIASDYKYKEAHEKQKGHYIGCRTAKEDPKLSWAARAMLLQNDRIYRKAYNDSKAHIHMPVDAMSLQAAKECQTLVSDV
DYRHYLHQWTCLPDQNDVMHARKAYDLQSDNVYKSDLEWLRGIGWLTEGSVDVIKAKKAQEILSDRLYRTQPDKMKFTSITDTPDVVQAKINA
MQLSNHLYREVWDKDKTQISIPSDTPELLQSKLNALNISNKHYQKAWDEAKAKNYDLRADAIPIKHAKASRDIASEYKYKEAHEKQKGHYIGC
RTAKEDPKLSWAARAMLLQNDRIYRKAYNDSKAHIHMPVDAMSLQAAKECQTLVSDVDYRHYLHQWTCLPDQNDVMHARKAYDLQSDNVYKSD
LEWLRGIGWLTEGSVDVVKAKKAQEILSDRLYRTQPDKMKFTSVTDAPDVVQAKINAMQLSNRLYREAWDKDKTQISIPSDTPEMLQSKVNAL
NISNKHYQKAWDEAKAKNYDLRADAIPIKHAKASRDIASEYKYKEAHEKQKGHYIGCRTAKEDPKLSWAARAMLLQNDRIYRKAYNDSKAHIH
MPVDAMSLQAAKECQTLVSDVDYRHYLHQWTCLPDHNDVVHARKAYDLQSDAVYKSDLEWLRGIGWLPNDSPGVQRVKHAQDLLSDKVYRTPI
DSVKYTSVVDSPDILLAKMNAEQLSIPKYKEAWEKDKTMIHIMPDTPEITLARSNAHNYSQKLYKEAWDEVKMSYDLRADAIPIKAAKASREI
ASDYKYKLDHEKQKGHYVGVPNAKADTKIRFALGIGKVQSELEYKKHFAKWKTQCHLPVDMLSIQSAKHGQSLVSDVDYRHYLHQWICLPDQN
DVIHARKAYDLQSDAVYKSDLEWLRGIGWLPNDSLGINHVKHAGDLLNERKYRTKAETLHFTPVADRVDYVTAKKSGEILSDIKYHKDWNEVK
SNYTLTDTPQLDMAREAARILNQSLYKESWEKEKATGYLLPPDTVQIRHAKHSNDVQSELKYKADYVKQRGHYVGVASMRDDPKLVWFEHAGE
IQNDRLYKSNYHKTKSKIHIPADIMSVVAAKECQALVSDVDYRHYLHQWTCHPDQNDCIQARKAYDLQSDNIYKSDLEWLRGCGWIPLGSVEH
KKVKHAQELINKRAYTKDAIENFSKYTSVVDTPDIVLAKINSVNQSDLKYKETFNLEKGQYIGSDDTPELNHARDMSLLYSDKLYKRDWEVCK
PIGYTLDAKYIPLVGAKHANYVNSELKYKEIYEKLKGHYLAGKDIGDFPSVVHSLAFQKIRSALAYRKNYEDTKTRVHIPSDMMNHVLAKKCQ
YILSDLEYRTYLHHWNCSPEEHDVIQARRAQEILSDVVYKDDLNWLKGIGCYVWDTPQILHAKKSYDLQSQIKYTAAGKENFQNFGVVTDTPV
YVTAVQSGINASDVKYKEDYHKTKDKYTTVTETADSERVQNLKHLFSNNLYKEAWDRVKATSYIMPSDAVSLARAKELKHNASIVKYREEYDK
FKALYTLPRSVEDDPNTARCLRVGKLNIDRLYKETYEKNKAKVHIIPDMVDIIAAKDAQKKISEIDYRTHLHDWICLPDLQINAHVRKVADQI
SDVVYKDDLNWLKGIGCFVWDTPEILHAKHAYDLRSDIKYKSDADKMKSKYTVVMDTPVYVQNILSGLNASEVIYRGDYLKKVRGKMIPTDKT
VDLQRAHHANKIQSENLYRWAGLKALPTGYSLPKDTPGFQHAKHVQHIGSDLKYKEAYEHMKAKGYTLGPNDVGFENVKKVNQVINERLYRAT
YHKNKDKIHTTPDTPEIRQVRATQEAVSDLIYKSDFFKLQGHLISLPFTPQVLHCRYVGDITSDNKYKEDLKWLQGLGCFLYDTPDMVRARQL
RKLWSNYVYTDSAKKMRDKYSVVLDTPGYRTVQELKTHLSDLVYRAAGKELKTKYTSVLNTPDFLRAKEGQRIQSQYLYVALATKERPHHHAG
NQTPAFTHARHVKDMVSETKYKIQYEKMKDKYTPVLDTPILIRAKRAYLNASDLRYKETFENTKGKYHTVKDALDIVYHRKVTDDISSVKYKE
NYMSQLGIWRSIPDRPEHFHHRAVTDAISDVKYKEDLSWLRGIGCYAWDTPDFALAEKNKVLYSGHKYKETFEKTKSHFKYVADSPINRHFKH
ATQLLDANSYKSLAKMLLKQGCNEILRPDILTALYNSYLWSQVEYKKDYEKKKDKYTTVVDTPENIRTAKVNKQISDIIYKLEYNKAKPKGYT
TIHDTPMLLHVRKVKDRISDLKYKELYERNKSHCNVVADSVHIKTPRHAYKLNSNLDYKKKYEAAKAHWHWIADRPDFVQAAKSSLQQSDYEY
KLDREYLKGCKLSVTDDKNTVLALNNAILASDIKYKEKHNKARGTCLVVPDTPQILLAKNVSSLVSENKYKEHSKKQLPRGSYTTLPETRDTA
HVKEVTKNVSDTNYKKKFVKEKGKSNYSIMLEPPEVKHAMEVAKKQSTVEYKKDAKSKLHYTPIADRPDIKKATQAAKLISDIEYKKRGEAGL
GVTMLGRPDIELAKEVSKLTSQAEYLKRHMRGHGAASYDTPWMRTFKKANMLSSHVKYKENFSKEKGKKPKYDLKEAKIYKTMKDAHNIASEV
KYKADLKKLHKPVTDMSESLIMNHVLNTSQLASAYQYKKQYEKSKGHYHMVPDNLEQVHLREASELQSHVKYKEKYEKEKGKPMLDFETPTYL
TAKESQLMQSEKEYKRDFEESIKGRNLTGLEITPSLLHVKYATKIASEKEYRKDLEEGVKGKGLSVLEETPDMLRAKNATQILNEKEYKKALE
LEIKGKGLSELALETPDFVRAKNATDIASQIKYKQLAEMEKANYTSVVDTPEIIHAQQVKNLSSQKKYKEEAEKTMPYYVPVADTPEMQRVRE
NQKNFSTLQYQWDLQNSKGKVTVVQDTPEMLRVKENQKNFSSILYKEDIGTGTTIEKTPEMQRVKRTQDAISSIKYKESIGKGTPIPDLPEVK
RVKETQKHISSVLYKEDLGTGIPTPVTPEIERVKRNQEHISSVLYKEELGTGTPIPVTPEVERVKRNQEHISSVLYKESVGTGTPTPITPEME
RVKRNQEICSSVLYKENIGKATPTPVTPEMERVKRNQEIISSVLYKENVGKVTPTPITPEMERVKRNQEIISSVLYKESLGRATPTPITPEME
RVKRNQEQISSVVYKEGLGKATPTPVTPEMERVRRNQEQISSVLYKEHLAKGTPTPMTPEMERAKRNQENISSVLYSDSFRKQVQGKAAYVLD
TPEMRRVRETQKHISTVKYHEDFEKNKGTFTPVVTDPITERVKKNMHDFSDISYRGIQRRVVEMEQKRVDQDQENLTGLRVWRTNPGSVFDYD
PAEDNIQSRSLHMISVQAQRRSREHSRSASALSISGGDEKSEPSEGVNQHLSYYSSGGFFTTTATVGYKHAKTIELPQQRSASVATQQTTVSS
VPSHPSTAGKTYRAMYDYTAADADEVSFKDGDTIVNVQAIDEGWMYGTVQRTGKTGMLPANYVEAV (SEQ ID NO: 65)
>tr|F1MQI3|F1MQI3_BOVIN Nebulin OS = Bos taurus OX = 9913 GN = NEB PE = 4 SV = 2
MADDEEYEEVVEYYTEETVYEEVPGETRTRFYETTTTRTSDYEQSETSRPALAQPVPEKPVERKRVIRKKVDPSKFMTPYIAHSQ
KMQNLFSTNKYKENYEKAKGKPYAITTDTPELRRIKKVQDQLSEVKYRVDGDVAKTICHVDEKAKDIEHAKKVSQQVSKVLYKQNWEDTKDKY
LLPPDAPELVQAIKNTAMFSKKLYTEDWEADKTMFYPYNDSPELRRVAQAQKALSDIAYKKGLAEQQTQFTSLPDPPDIEFAKKVTNQVSKQK
YKEDYENKVKGKWSETPCFEIATARMNSNNISTKKYQEDFEHMKDQIYFMQTETPEYKVNKQAGVAASKVKYKQDYEKNKGKADYNVLPASEN
PLLRQLKAAGDALSDKLYKENYEKTKAKSINYCETPKFKLDTVLHNFSSDTKYKDSYLKDILGHYVGSYEDPYHTHCMRVSAQNSDKNYKAEY
EEDRGKGFFPQTITQEYEAIKKLDQCKDHTYKVHPDKTKFTQVTDSPVLMQAQVNSKQLSDLNYKAKHENEKFKCHIPPDTPAFIQHKLNAYN
LSDNIYKHDWEKTKAKKEDIKVDAIPLLAAKANTKIASDVMYKKDYEKSKGKMIGALSINDDPKMLHSLKTAKNQSDRLYKENYEKTKAKSMN
YCETPKYQLDTLLKNFSEAKYKDSYVQNVLGHYIGSFEDPYQVHCLKISAQNSDKNYKAEYEEDKGKCYFPQTITQEYEAIKKLDQCKDHTYK
VHPDKTKFTAVTDTPVLLQAQLNTKQLSDLNYKAKHEGEKFKCHIPADAPQFIQHRVNAYNLSDNIYKHDWEKTKAKKFDIKVDAIPLLAAKA
NTKIASDVMYKKDYEKSKGKMIGALSINDDPKMLHSLKTAKNQSDHEYRKDYEKSKTIYTAPLDMLPLTHAKKSQAIASDVDYKHLLHNYSYP
PDSVNVDLAKKAYALQSDVEYKADYNSWMKGCGWMPFGSLEMEKAKRASDILNEKKYRQHPDTLKFTSIEDAPIIVQSKINQAQRSDVAYKAK
GEEVIHKYSLPADLPQFIQAKVNAYNISENLYKADLKDLSKKGYDLRIDAIPIKAAKAARQAASDVQYKKDYEKAKGKMVGFQSLQDDPKLVH
YMNVAKIQSDREYKKAYEKTKTRHNTPHDMVNIVAAKKAQDVASNVNYKHSLHHYTYLPDAMDLELSKNMMHIQSDNVYKEDYNNWMKGIGWI
PIGSLEVEKVKKAGDALNEKKYRQHPDTLKFTSIVDSPVMVQAKQNTQQVSDILYKAKGEDVKHKYTMSPDLPQFLQAKCNAYNLSDVCYKRD
WHDLIAKGTNVLGDAIPITAAKASRNIASDYKYKEAYEKAKGKQVGFRSLQDDPKLVHYMNVAKLQSDREYKKNYENTKTSYHTPGDMVSITA
AKMAQDVATNVNYKQPIHHYTYLPDALSLEHIRNVNQIQSDNVYKDEYNHFFKGMGWIPIGSLEVEKVKKAGDALNEKKYRQHPDTIKFTSVP
DSMGMVLAQHNTKQLSDLNYKVEGEKVKHKYTMDPDVPQFIQAKVNAYNMSDSHYKADWKKTLAKGYDLRPDAIPIVAAKSSRNIASDFKYKE
AYEKTKGKQIGFRSLQDDPKLVHFMNVAKMQSDREYKKGYEASKTKYHTPLDMLSVTAAKKSQEVATNTNYKQPFHHYTLLPDALNVEHSRNA
MQIQSDNLYKSDFTNWMKGIGWLPLESLEVEKAKKAGEILSEKKYRQHPEKLKFTYAMDTMEQALNKSNKLIMDKRLYTEKWNKDKTTIHVMP
DTPDILLSRVNQITMSDKLYKAGWEEEKKKGYDLRPDAISIKAARASRDIASDYKYKQAYEQAKGKQIGFRSLEDDPKLVHFMQVAKMQSDRE
YKKAYEKSKTSFQTPVDMLSVVAAKKSQEVATNANYRNVIHTYNMLPDAMSLELAKNMMQIQSDNQYKADYADFMKGIGWLPLGSLEAEKNKK
AMEIISEKKYRQHPDTLKYSTLMDSMNMVLAKNNAKIMNEHLYKQAWEADKTKVHIMPDIPQIILAKANAINISDKLYKLSLEEAKKKGYDLR
TDAIPIKAAKASRDIASDYKYKHSYEKERGKMVGFRSLEDDPKLVHSMQVAKMQSDREYKKNYEKTKTSYHTPADMLSVTAAKDAQANITNTN
YKHLIHKYILLPDAMNIQLSKNMNRIQSDNEYKQDYNEWYKGLGWSPAGSLEVEKAKKATEYASDQKYRQHPSNFQFTKLNDSMDMVLAKQNA
HTMNKYLYTVDWNKDKTKIHVMPDTPDILQAKQNQTMYSQVKYKMGWPSGLERVVNLPSVLIKIFKSKQNKDLIWRFKYKQGYRKQIGHHIGF
RSLQDDPKLVLSMNVAKMQSEREYKKDFEKWKTKFSSPVDMLGVVLAKKCQALVSDVDYKNYLHGWTCLPDQNDVIHAKKAYDLQSENLYKSD
LEWLKGIGWSPLGSLEAEKNKRASEIISEKKYRQPPDRNKFTSIPDAMDIVLAKTNAKNRSDILYREAWDKDKTQIHIMPDTPDIILAKANLI
NTSDKFYRMGYEELRKKGYDLPVDAIPIKAAKASREIASEYKYKEGFRMQLGHHIGARNIKDDPKMMWSMHVAKIQSDREYKKDFEKWKTKFS
SPVDMLGVVLAKKCQTLVSDIDYKNYLHQWTCLPDQSDVIHARRAYDLQSDNLYKSDLQWLRGIGWLPSGSLEDEKNKRASQILSDHVYRQHP
DQFKFSSLMDSIPMVLAKNNAITMNHRLYTEAWDKDKTTVHIMPDTPEVLLAKQNQINYSEKLYKLGLDEAKRKGYDMRIDAIPIRAAKASRD
IASEFKYKEGYRRQLGHHIGARAIHDDPKMMWSMHVAKIQSDREYKKDFEKWKTKFSSPVDMLGVVLAKKCQTLVSDIDYKNYLHQWTCLPDQ
SDVIHARQAYDLQSDNVYKSDLQWLRGIGWVPIGSLDVEKSKRASEILSDKLYRQPPDKFKFTSVTDSLEQVLAKNNAINMNKRLYTEAWDKD
KTQVHIMPDTPEITLARTNKVNYSENLYKLAHEEAKKKGYDLRSDAIPIIAAGLEKDHISDYKYKDGYRKQLGHHIGARDIKDDPKMMWSMHV
AKIQSDREYKKDFEKWKTKFSSPVDMLGVVLAKKCQTLVSDIDYKNYLHQWTCLPDQSDVIHARQAYDLQSDNVYKSDLQWLRGIGWVPIGSV
DVVKCKRAAEILSDNLYRQPPDTLKFTSVPDSLEQVLAKNNAINMNKRLYTEAWDKDKTQIHIMPDTPEIMLARQNKLNYSETLYKLANEEAK
KKGYDLRSDAIPIVAAKASRDIISDYKYKDGYRKQLGHHIGARDIKDDPKMMWSMHVAKIQSDREYKKDFEKWKTKFSSPVDMLGVVLAKKCQ
TLVSDIDYKNYLHQWTCLPDQSDVIHARRAYDLQSDNIYKSDLQWLRGIGWVPIGSVDVVKCKRAAEILSDNLYRQRPDTLKFTSVPDSLEQV
LAKNNAINMNKRLYTEAWDKDKTQIHMMPDTPEITLARQNKINYSENLYRQAMEEAKKEGYDLRSDAIPIVAAKASREIASDYKYKEAYRKQL
GHHIGARAIHDDPKMMWSVHVAKMQSDREYKKDFEKYKTRESSPVDMLGIVLAKKCQTLVSDVDYKHPLHEWTCLPDQNDIIHARKAYDLQSD
NLYKSDLEWLKGIGWVPIGSVEVLKAKRAGEILSDNIYRQRPDTLKFTSVTDSPEQVLAKNNAINMNKRLYTEAWDNDKKTIHVMPDTPEIML
AKLNRINYSDKLYKLALEESRKEGYDLRLDAIPIQAAKASREIASDYKYKEGYRKQLGHHIGARNIKDDPKMMWSIHAGKLQSDLEYKKDFEK
WKTKFSSPVDMMGLVQAKKCQILVSDIDYKNLLHEWTCLPDQNDIIQARKAYDLQSDAIYKADLEWLRGIGWVPIDSVGVEHAKRAGEILSER
KYRQPAVQLKFTSITDTPEIVLAKNNALNVSKHLYTEAWDADKTSIHVMPDTPEILLAKSNSANISHKLYTKGWDESKMKDYDLRADAISIKS
AKASRDIASDYKYKEAYEKHKGHHIGARSVEDDPRIMCAMNAGRIQSEREYKKEFQKWKTKFSSPVDMLGILLAKKCQTLVSDIDYRNYLHHW
TCLPDQNDIIQARKAYDLQSDAIYKADLEWLRGIGWMPEGSPEVLRVKNAQHIFRDSVYRTPVVKLKYTSIVDTPEVVLAKSNAENISIPKYR
EVWDKDKTSIHIMPDTPEINLARTNALNVSNKLYREGWDEMKMSCDVRLDAIPIQAAKASREIASDYKYKLDHEKQKGHYVGTRTARDDNKIR
WALIAGKIQNEREYRLHWAKWKSKFQSPPDMLSIEHSKNSQTLVSDIDYRHYLHQWTCMPDQNDVIQARKAYDLQSDAIYKADLEWLRGIGWM
PADSVSVNHAKHMADIFNEKKYRTKIETLSFTPVDDRVDYVTAKHSGEILNDIKYRKDWNDTKSKYTLTETPQLHTAQEAARILDQYLYKESW
EKQKATGYILPPDAVPFVHAHHSGDVQSELKYKAEHVKQKGHYVGVPTMRDDPKLVWFEHAGQIQNDRLYKENYHKTKAKIHIPPDMVSVLAA
KEGQALASDIDYRNYLHQWICHPDQNDVIQARKAYDLQSDNIYKADLEWLRGIGWIPLDSVDHVRVTRNQEMMNQIKYKKDALANYPNFTSVV
DPPEIVLAKINSVNQSDVKYKETFNKLIKGKYIFSPDTPYITHSKDMEKLYSTILYKRAWDGTKAYGYTLDERYIPIVGAKHADFVNSELKYK
ETYEKLKGHYLAGKEISEFPNVVHCLDFQKMRSLLNYRRHYEDTKANVHIPQDMMNHVLAKRCQYILSDLEYRHYFHQWTSLPEEPNVVRVRH
AQEILSDNVYKDDLNWLKGIGCYVWDTPQILHAKKSYDLQSQILYTAAGKENLKNYNLVTDTPLYVTALQSGINASEVKYKENYHQTKDKYTT
VLETVDYDRIKNLKDLFSSNLYKEAWDKVKATSYILPPNTVSLTHAKNQKYMASHIKYREEYEKFKALYTLPRSVEDDPNTARCLRVGKFNID
RLYRSVYEKNKMKIHIVPDMVEMVTAKDSQKKVSEIDYRLHLHEWICHPDLQVNSHVRKVTDQISDIVYKDDLTWLKGIGCYVWDTPEILHAK
HAYDLRNDIKYKAHVLKTRNNYKLVTDTPVYVQAVKSGKQLSDAVYHYDYVHSIRGRVAPTTKTVDLDRALHAYKLQSENLYRKAGLHALPTG
YRLPVDTPHFKHTKDTRYMSSYFKYKEVYEHMKAYGYTLGPNDVPFVNVRRVNNITSERLYRQLYHKLKDKIHTTPDTPEIRQVKKTQEAVSE
LIYKSDFFKMQGHMISLPYTPQVLHCRYVGDITSDIKYKEDLQVLRGMGCFLYDTPDMVRSRHLRKLWSHYLYTDKARKMRDKYKVVLDTPEY
RKVQELKTHLSELVYRAAGRKQKSIFTSVPDTPDLTRAKRGQKLQSQYLYVELATKERPHHHAGNQTTALKHARHVKDMVSENKYKIQYEKMK
DKYTPVPDTPILIRAKRAYWNASDLRYKETFQKTKGKYHTVKDALDIVYHRTVTDHISKIKYKENYMSQLGIWRSIPDRPEHFHHRAVTDAVS
DVKYKQDLTWLKGIGCYAYDTPDFTLAEKNKTLYSKYKYKEVFERTKSNFKYVADCPINRHFKFATQLMNEKKYRADYEQRKDKYHLVVDEPR
HLLAKIAGDQISQIKYRKNYEETKDKFTSIVDTPEHLRTTKVNKQISDILYKLEYNKAKPRGYTTIHDTPMLLHVRKVKDEVSDLKYKEVYQR
TKSNCTIEPDAVHIKAAKDAYKVNTNLDYKKKYEATKAYWKWTPDRPDFIQAAKSTLQQSDFEYKLDREYLKGCKLSVTDDKDMVLALKNSII
ESDLKYKEKHVKERGSCHAVPDTPQILLAKTVSSLVSENKYKSYVKKHLAQGSYTTLPETRDTIHVKEVTKNVSDTNYKKKFVKEKGKSNYSI
MLEPPDVKHAMDVAKKQSNVAYKKDAKENLHYTTVADRPDIKKATQAAKQASEVEYRAKHRKEGSHGLSMLGRPDIEMAKKAAKLSSQVQYRE
NFNKEKGKTPKYNPKDSQLYKVMKDANTLASEVKYKADLKKLHKPVTDMKESLIMNHVLNTSHLASSYQYKKNYEKSKGHYHTIPDNLEQLHL
KEATELQSIVKYKEKYEKERGKPMLDFETPTYITAKESQQMQSGKEYRKDYEESIKGRNLTGLEVTPALLHVKYATKIASEKEYRKDLEESIR
GKGLSEMEDTPDMLRAKNATQILNEKEYKRDLELEVKGRGLNAMANETPDFLRARNATDIASQIKYKQSAEMEKANFTSVVDTPEIIHAQQVK
NLSSQKKYKEDAEKCMSYYETVLDTPEMQRVRENQKNFSLLQYQYDLKNSKGKITVVQDTPEILRVKENQKNFSSVLYKEDVSPGTAIGKTPE
MMRVKQTQDHISSVKYKEVIGQGTPIPDLPEVKRVKQTQKHISSVMYKENLGTGIPTPVTPEIERVKRNQENFSSVLYKENLGKGTPTPITPE
MERVKRNQENFSSILYKENLSKGTPLPVTPEMERVKRNQENFSSVLYKENVGKGTPTPVTPEMQRVKRNQENISSVLYKENLGKATPTPFTPE
MERVKRNQENFSSVLYKENMRKATPTPVTPEMERAKRNQENISSVLYSDSFRKQIQGKAAYVLDTPEMRRVRETQRHISTVKYHEDFEKHKGC
FTPVVTDPITERVKKNTQDFSDICYRGIQRKVVEMEQKRNDQDQETITGLRVWRINPGSVFDYDPAEDNIQSRSLHMINAQAQRRSREQSRSA
SGLSISGGEEKSEHSEAAHLSTYSDGGVFFSAASTGYKHARTTELPQQRSSSVATQQTTVSSIPSHPSTAGKIFRAMYDYMAADADEVSFKDG
DAIVNVQAIDEGWMYGTVQRTGRTGMLPANYVEAI (SEQ ID NO: 66)
>tr|F1MPU4|F1MPU4_BOVIN Myotilin OS = Bos taurus OX = 9913 GN = MYOT PE = 4 SV = 1
MFNYERPKHFIQSQNPCGSRLQPPGPEISSYSSQTKQSSITIQPRQCTEQRYSASSTVSSHITMSSSAFPASPQQLAGSNPAQRV
TATYNQSPASFLSSILPSQPDYSSSKNPSTVDSNYQQPSVGQPINVKSSQNANAKLTPKTPDHEIQGSKEALIQDLERKLKCKDSLLHNGNQR
LTYEEKMARRLLGPQNAAAVFQGQNDSEAQDSAQQHNIEHARLQVPTSQVRSRSSSRGDVNDQDAIQEKFYPPRFIQVPENMSIEEGRFCRMD
FKVSGLPAPDVSWYLNGRPVQSDDFHKMIVSEKGFHSLIFEVVRASDAGAYACVAKNRAGEATFTVQLDVLAKEHRRAPMFIYKPQSKKVFEG
ESVKLECQISAVPPPKLFWKRNNEMVQFNTDRISLYHDNSGRVTLLIKDVNKKDAGWYTVSAVNEAGVTTCNTRLDVTARPNQTLPAPKQLRV
RPTFSKYLALNGKGLNVKQAFNPEGEFQRLAAQSGLYESEEL (SEQ ID NO: 67)
>tr|A0A1D5NT92|A0A1D5NT92_CHICK Myotilin OS = Gallus gallus OX = 9031 GN = MYOT PE = 4 SV = 1
MSVRNLPSVSSMCQPTMFNYERPKHFIQSKNVCQGQQQPPGSSTSTESSRQIKQSSILIQPRNPSGQKFSSSSSLSSSITLSSPS
CSAPKESTYPVTPASAQSPASSSSGQRLISMPNQTPAAFLCSVLPSQPDYNSQTPPMEPHYSKPMYKKQASINSMQKTSDQEIRGTKEALIQD
LEKKLRCKDNLLQNGNQRLTYEERMARRLLGPENAASVLEAQSEDMQNTQNAENVRLQVPTTHVRSRPSSRGDERGHDSIQEKFFQPRFTQVP
EDVIIEEGRFCRLDFKVSGLPTPDVMWYQNGRMVHQDQFHKMIVSEKGFHSFIFEAVKSSDAGTYECVAVNRAGESSFAVKVEVIAKEHHTPP
TFIFKPQSKKVFEGDTARLECQISAIPTPRIYWKRNNEMVQYNTDRISLLHDNTGRICLLIHNANKKDAGWYTVSAVNGAGVATCHARLEVAT
HTNKPVPAPKQLRVRPTFSKYLALNGRGLDVKQAFSPEGEFQRLAEQSGLYESDEL (SEQ ID NO: 68)
>tr|F1RH92|F1RH92_PIG Myotilin OS = Sus scrofa OX = 9823 GN = MYOT PE = 4 SV = 1
MFNYERPKHFIQSQNPCGSRLQPPGPETSSYSSQTKQSSIIIQPRQCTEQRFSASSTVSSHITMSSSAFPASPQQPAASNPGQRV
TATYNQSPASFLSSILPSQPDYSSSKIPSTMDSNYQQPSVGQPVNAKPSQSLNAKPIPRTPDHEIQGSKEALIQDLERKLKCKDSLLHNGNQR
LTYEEKMARRLLGPQNAAAVFQAQNDSAAQDSPQQHNSEHARLQVPTSQVRSRSSSRGDVNDQDAIQEKFYPPRFIQVPENMSVEEGRFCRMD
FKVSGLPAPDVSWYLNGRPVQSDDLHKMIVSEKGFHSLIFEVVRASDAGAYACVAKNRAGEATFTVQLDVLAKEHRRAPMFIYKPQSKKVFEG
DSVKLECQISAIPPPKLFWKRNNEMVQFNTDRISLYHDNSGRVTLLIKDVNKKDAGWYTVSAVNEAGVITCNTRLDVTARPNQTLPAPKQLRV
RPTFSKYLALNGRGLDVKQAFNPEGEFORLAAQSGLYESEEL (SEQ ID NO: 69)
>XP_013837221.1 twinfilin-2 [Sus scrofa]
MAHQTGIHATEELKEFFAKARAGSVRLIKVVIEDEQLVLGASRELMGCWDQDYDRAVLPLLDAQQPCYLLYRLDTQNAQGFEWLF
LAWSPDNSPVRLKMLYAATRATVKKEFGGGHIKDELFGTVKDDLSFAGYQKHLSSCAAPAPLTSAERELQQIRINEVKTEISVESKHQTLQGL
AFPLQPQAQRALQQLRQKMINYIQLKLDLERETIELVHTEPTDVAQLPSRVPRDAARYHFFLYKHTHEGDPLESVVFIYSMPGYKCSIKERML
YSSCKSRLLDSVEQDFQLEIAKKIEIGDGAELTAEFLYDEVHPKQHAFKQAFAKPKGPGGKRGHKRLIRGPGENGDDS (SEQ ID NO:
70)
>XP_024844129.1 dystrophin isoform X4 [Bos taurus]
MLWWEEVEDCYEREDVQKKTFTKWINAQFSKFGKQHIENLFSDLQDGRRLLDLLEGLTGQKLPKEKGSTRVHALNNVNKALQVLQ
KNNVDLVNIGSSDIVDGNHKLTLGLIWNIILHWQVKNVMKNIMAGLQQTNSEKILLSWVRQSTRNYPQVNVINFTTSWSDGLALNALIHSHRP
DLFDWNSVVRQQSATQRLEHAFNIAKYQLGIEKLLDPEDVATTYPDKKSILMYVTSLFQVLPQQVSLEAIQEVEMLPRPSKVTREEHFQLHHQ
MHYSQQITVSLAQGYERTPSSPQPRFKSYAYTQAAYVSTSDPTRSPFPSQRLEAPEDKSFGSPLMETEVNLDSYQTALEEVLSWLLSAEDTLQ
AQGEISNDVEEVKEQFHTHEGYMMDLTSHQGRVGNVLQLGSQLIGSGKLSEDEETEVQEQMNLLNSRWECLRVASMEKQSNLHKVLMDLQNQQ
LKELNAWLTKTEERTRKMEKEPLGPDLEDLKRQIQQHKVLQEDLEQEQVRVNSLTHMVVVVDESSGDHATAALEEQLKVLGDRWANICRWTED
RWVLLQDVLLKWQRFTEEQCLFSTWLSDKEDALNKIPTSGFKDQSEMLSSLQKLAVLKTDLEKKKQTMDKLCSLNHDLLSTLKNTLVAQKMEA
WLDNFAQRWDNLVQKLEKSSTQISQAVTTTQPSLTQTTVMETVTMVTTREQILVKHAQEELPPPPPQKKRQIIVDSEIKKRLDVDITELHSWI
TRSEAVLQSPEFAVYRKEGNFSDLKEKVNAIEREKAEKFRKLQDASRSAQALVEQMVNEGVNADSIKQASEQLNSRWIEFCQLLSERLNWLEY
QNNIITFYNQLQQLEQMTTTAENWLKTQPTTPSEPTAVKSQLKNCKDEVNRLSALQPQIERLKIQSIALKEKGQGPMFLDADFVAFTNHFNQV
FADMQAREKELQTILDTLPTVRYQETMSTILTWIQQSETKLSIPQVTVTEYEIMEQRLEELQALQSSLQEHQNDLNYLSTTVKEMSRKAPSHI
SQRYQSEFENIEGRWKKLSAQLNERCQKLEEQMAKLRKLQNHIKTLKKWMAEVDVFLKDEWPALGDSEILKKQLKQCRLLVSDIQTIQPSLNS
VNEGGQKIKTEAEPEFASRLETELRELNTQWDYICRQVYARKEALKGGLDKTVSLQKDLSEMHEWMTQAEEEYLERDFEYKTPDELQTAVEEM
KRAKEEAQQKEAKVQLLTESVNSVIAQAPPAAQEALRKELDTLTTNYQWLCTRLNGKCKTLEEVWACWHELLSYLEKANKWLNEVELKLKTTE
NIPGGAEEISEVLSSLENLMQHSEDNPNQIRILAQTLTDGGVMDELINEELETFNSRWRELHEEAVRRQKLLEQSIQSAQEIEKSLQLIQESL
SSIDKQLAAYIADKVDAAQMPQEAQKIQSDLTSHEISLEEMKKHYQGKETAQRVLSQIEVAQKKMQDVSMKFRLFQKPANFEQRLQESKMILD
EVKMHLPALETKSVEQEVVQSQLNHCVNLYKSLSEVKSEVEMVIKTGRQIVQKKQTENPKELDERVTALKLHYNELGAKVTERKQQLEKCLKL
SRKMRKEMNALTEWLAATDLELTKRSAVEGMPSNLDSEVAWGKATQKETEKQKVHLKSITELGEALKTVLGKKETLVEDKLSLLNSNWIAVTS
RAEEWLNLLLEYQKHMETFDQNVDHITKWIIQADALLDESEKKKPQQKEDMLKRLKAELNDIRPKVDSTRDQAANLMANRGDHCRKVIEPKIS
ELNHRFAAISHRIKTGKASIPLKELEQFNSDIQKLLEPLEAEIQQGVNLKEEDFNKDMSEDNEGTVKELLQRGDNLQQRITDERKREEIKIKQ
QLLQTKHNALKDLRSQRRKKALEISHQWYQYKRQADDLLKCLDDIEKKLASLPEPRDERKIKEIDRELQKKKEELNAVHRQAEGLSEDGAAMA
VESTQIQLSKRWREIESKFAQFRRLNFAQIHTVHEESVMVMTEDMPLEISYVPSTYLTEITHVLQALSEVEQLLNAPDLCAKDFQDLFKQEES
LKNIKDNLQQISGRIDVIHNKKAAALQSTTPPEKARLQEALSRLDFQWERVNKMYKDRQGQFDRSVEKWRRFHYDMKIFNQWLTEAEHFLKKT
QIPENWEHAKYKWYLKELQDGIGQRQTVVRVLNATGEEIIQQSSKIDASILQEKLGSLNLRWQEVCKQLAERKKRLEEQKNILSEFQRDLNEF
VLWLEEAGNISSIPLEPGNEQQLKEKLEEVKLLAEELPLRQGILKQLNETGGTVLVSAPISPEEQDKLENKLKQTNLQWIKVSRSLPEKQGEI
EAHIKDLGQLEEQLNHLLLWLSPIRSQLEIYNQPNQTGPFDIKETEVAVQAKQPDVEGILSKGQNLYKEKPATEPVKRKLEDLSSEWKAVTHL
LQELRAKWPGPVPGLTATGAPPSQTVALVTQPVVAKETAVSKLEMPSSLLLEVPALADFNRAWTELTDWLSLLDRVLKAQRVMVGDLEDINEM
IIKQKATLQDLEQRRPQLEELITAAQNLKNKTSNQEARTIITDRIERIQSQWDEVQEHLQNRRQQLNEMLKDSTQWLEAKEEAEQVVGQARAK
LETWKEGPYTMDAIQRKITETKQLAKDLRQWQINVDVANDLALKLLRDYSADDTRKVHMITENINASWASIHKRVSERETALEETHRLLQQFP
LDLEKFLAWLTEAETTANVLQDATHKERLLEDSKGVRELMKQWQDLQGEIEAHTDIYHNLDENGQKILRSLEGSDDAVLLQRRLDNMNFKWSE
LRKKSLNIRSHLEASSDQWKRLHLSLQELLVWLQLKDDELSRQAPIGGDFPAVQKQNDIHRAFKRELKTKEPVIMSTLETVRIFLTEQPLEGL
EKLYQEPRELPPEEKAQNVTRLLRKQAEEVNTEWEKLNLHSADWQRKIDEALERLQELQEATDELDLKLRQAEVIKGSWQPVGDLLIDSLQDH
LEKVKALRGEIAPLKENVSHVSDLARQLTTLGIQLSPYNLSTLEDLNTRWKLLQVAVEDRIRQLHEAHRDFGPASQHELSTSVQGPWERAISP
NKVPYYINHETQTTCWDHPKMTELYQSLADLNNVRFSAYRTAMKLRRLQKALCLDLLSLSAACDALDQHNLKQNDQPMDILQIINCLTTIYDR
LEQEHNNLVNVPLCVDMCLNWLLNVYDTGRTGRIRVLSFKTGIISLCKAHLEDKYRYLFKQVASSTGFCDQRRLGLLLHDSIQIPRQLGEVAS
FGGSNIEPSVRSCFQFANNKPEIEAALFLDWMRLEPQSMVWLPVLHRVAAAETAKHQAKCNICKECPIIGFRYRSLKHFNYDICQSCFFSGRV
AKGHKMHYPMVEYCTPTTSGEDVRDFAKVLKNKFRTKRYFAKHPRMGYLPVQTVLEGDNMETPVTLINFWPVDSAPASSPQLSHDDTHSRIEH
YASRLAEMENSSGSYLNDSISPNESIDDEHLLIQHYCQSLNQDSPLSQPRSPAQILISLESEERGELERILADLEEENRNLQAEYDRLKEQHE
HKGLSPLPSPPEMMPTSPQSPRDAELIAEAKLLRQHKGRLEARMQILEDHNKQLESQLHRLRQLLEQPQAEAKVNGTTVSSPSTSLQRSDSSQ
PMLLRVVGSQTSESMGEEDLLSPPQDSSTGLEEVMEQLNNSFPSSRGRNTPGKPMREDTM (SEQ ID NO: 71)
>tr|Q05JF3|Q05JF3_BOVIN Calsequestrin OS = Bos taurus OX = 9913 GN = CASQ1 PE = 1 SV = 1
MSAADRMGARAVPGLRLALLLLMVLGTPKSGVQGEEGLDFPEYDGVDRVVNVNAKNYKNVFKKYEVLALLYHEPPEDDKASQRQFEMDELILE
LAAQVLEDKGVGFGMVDSEKDAAVAKKLGLTEEDSVYVFKGDEVIEYDGEFSADTLVEFLLDVLEDPVELIEGERELQAFENIEDDNKLIGYF
KNKDSEHYKAYEDAAEEFHPYIPFFATFDSKVAKKLTLKLNEIDFYEAFMEEPVTIPDKPNSEEEIVSFVEAHKRSTLRKLKPESMYETWEDD
LDGIHIVAFAEETDPDGYEFLETLKAVAQDNTDNPDLSIIWIDPDDFPLLVPYWEKTFNIDLSAPQIGVVNVTDADSVWMEMDDEEDLPSAEE
LEDWLEDVLEGEINTEDDDEEDD (SEQ ID NO: 72)
Cardiac muscle tissue
>sp|P68034|ACTC_CHICK Actin, alpha cardiac muscle 1 OS = Gallus gallus OX = 9031 GN = ACTC1
PE = 3 SV = 1
MCDDEETTALVCDNGSGLVKAGFAGDDAPRAVFPSIVGRPRHQGVMVGMGQKDSYVGDEAQSKRGILTLKYPIEHGIITNWDDMEKIWHHTFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILTERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVLSGGTTMYPGIADRMQKEITALAPSTMKIKIIAPPERKYSVWIGGSILASLSTFQQMWISKQEYDEAGPSIV
HRKCF (SEQ ID NO: 73)
>NP_990097.1 myosin heavy chain, cardiac muscle isoform [Gallus gallus]
MMDMTEFGEAAPFLRKSEKELMMLQTVAFDGKKKCWVPDDKKAYVEAEITESSGGKVTVETTDGRTMTIKEDDVQSMNPPKEDMIEDMAMLTH
LNEASVLYNLRKRYSNWMIYTYSGLFCVTINPYKWLPVYKSEVVAAYKGKRRSEAPPHIFSIADNAYHDMLRNRENQSMLITGESGAGKTVNT
KRVIQYFATVAALGEPGKKSQPATKTGGTLEDQIIQANPALEAFGNAKTLRNDNSSRFGKFIRIHFGTTGKLSSADIEIYLLEKSRVIFQQPG
ERDYHIFYQILSGKKPELLDMLLVSTNPYDYHFCSQGVVTVDNLDDGEELMATDQAMDILGFVPDEKYGAYKLTGAIMHFGNMKFKQRPREEQ
AEADGTESADKAAYLMGINSSDLVKGLLHPRVKVGNEYVTKGQSVEQVLYAVGALSKAVYDRMFKWLVVRINKTLDTKLPRQFFIGVLDIAGF
EIFDFNSFEQLCINYTNEKLQQFFNHHMFVLEQEEYKKEGIEWVFIDFGMDLQACIDLIEKPLGILSILEEECMFPKATDMTFKAKLYDNHLG
KSPNLQKPRPDKKRKYEAHFELIHYAGSVPYNIIGWLEKNKDPLNETVVGIFQKSSNKLLASLFESYVGADSADQGGEKKRKKGASFQTVSSL
HKENLNKLMTNLRSTAPHFVRCIIPNESKTPGEMDAFLVLHQLRCNGVLEGIRICRKGFPNRVLYADFKQRYRILNPGAIPEDKFVDSRKAAE
KLLASLDIDHNQYRFGHTKVFFKAGLLGHLEEMRDERLAKILTMIQARARGRLMRIEFQKIVERRDALLVIQWNIRAFMAVKNWPWMKLFFKI
KPLLKSAETEKEMANMKEEFLKLKEALEKSEARRKELEEKQVSLVQEKNDLLLQLQAEQDTLADAEERCDLLIKSKIQLEAKVKELTERVEDE
EEMNSELTSKKRKLEDECSELKKDIDDLEITLAKVEKEKHATENKVKNLTEEMATLDENISKLTKEKKSLQEAHQQVLDDLQAEEDKVNTLSK
AKVKLEQQVDDLEGSLEQEKKVRMDLERAKRKLEGDLKLTQESVMDLENDKLQMEEKLKKKEFEMSQLNSKIEDEQAIVMQLQKKIKELQARI
EELEEELEAERAARAKVEKQRSDLARELEVLSERLEEAGGATAAQLEMNKKREAEFLKLARDLEEATLHYEATAAALRKKHADSVAEMGEQLD
NLQRVKQKLEKEKSELKMEVDDLTSNMEQTVKGKANAEKLCRTYEDHLNETKTKLDEMTRLMNDLTTQKTKLQSENGEFVRQLEEKESLISQL
SRGKTSFTQQIEELRRQLEEETKSKNALAHALQAARHDCDLLREQYEEEQEAKAELQRALSKGNAEVAQWRTKYETDAIQRTEELEDAKKKLA
ARLQEAEEAIEAANAKCSSLEKTKHRLQNELEDMMIDLEKANSAAASLDKKQRGFDKIINDWKQKYEESQAELEASQKEARSLSTELFKLKNA
YEETLDHLETLKRENKNLQEEISDLINQISEGNKNLHEIEKVKKQVEQEKSEVQLALEEAEGALEHEESKTLRFQLELSQLKADFERKLAEKD
EEMENIRRNQQRTIDSLQSTLDSEARSRNEAIRLKKKMEGDLNEMEIQLSHANRHAAEATKSARGLQTQIKELQVQLDDLGHLNEDLKEQLAV
SDRRNNLLQSELDELRALLDQTERARKLAEHELLEATERVNLLHTQNTSLINQKKKLEGDISQMQNEVEESIQECRNAEEKAKKAITDAAMMA
EELKKEQDTSAHLERMKKNMEQTIKDLQKRLDEAEQIALKGGKKQIQKLESRVRELENELENELRRNSDAQKGARKFERRIKEVTYQSEEDKK
NLARMQDLIDKLQLKVKSYKHQAEEAEAQANLYLSKYRKQQHDLDDAEERAEIAESQVNKLRSKSRDIGMKKVHEEE(SEQIDNQ:74)
>sp|Q90688|MYPC3_CHICK Myosin-binding protein C, cardiac-type OS = Gallus gallus OX = 9031
GN = MYBPC3 PE = 1 SV = 3
MPEPAKKAVSAFTKKPKTTEVAAGSTAVFEAETEKTGIKVKWQRAGTEITDSEKYAIKAEGNKHSLTISNVGKDDEVTYAVIAGTSKVKFELK
VKEPEKSEPVAPAEASPAPAASELPAPPVESNQNPEVPPAETQPEEPVDPIGLFVTRPQDGEVTVGGNITFTAKVAGESLLKKPSVKWFKGKW
MDLASKVGKHLQLHDNYDRNNKVYTFEMEIIEANMTFAGGYRCEVSTKDKFDSSNFNLIVNEAPVSGEMDIRAAFRRTSLAGGGRRMTSAFLS
TEGLEESGELNFSALLKKRDSFLRTANRGDGKSDSQPDVDVWEILRKAPPSEYEKIAFQYGITDLRGMLKRLKRIKKEEKKSTAFLKKLDPAY
QVDKGQKIKLMVEVANPDADVKWLKNGQEIQVSGSKYIFEAIGNKRILTINHCSLADDAAYECVVAEEKSFTELFVKEPPILITHPLEDQMVM
VGERVEFECEVSEEGATVKWEKDGVELTREETFKYRFKKDGKKQYLIINESTKEDSGHYTVKTNGGVSVAELIVQEKKLEVYQSIADLTVKAR
DQAVFKCEVSDENVKGIWLKNGKEVVPDERIKISHIGRIHKLTIEDVTPGDEADYSFIPQGFAYNLSAKLQFLEVKIDFVPREEPPKIHLDCL
GQSPDTIVVVAGNKLRLDVPISGDPTPTVIWQKVNKKGELVHQSNEDSLTPSENSSDLSTDSKLLFESEGRVRVEKHEDHCVFIIEGAEKEDE
GVYRVIVKNPVGEDKADITVKVIDVPDPPEAPKISNIGEDYCTVQWQPPTYDGGQPVLGYILERKKKKSYRWMRLNFDLLKELTYEAKRMIEG
VVYEMRIYAVNSIGMSRPSPASQPFMPIAPPSEPTHFTVEDVSDTTVALKWRPPERIGAGGLDGYIVEYCKDGSAEWTPALPGLTERTSALIK
DLVTGDKLYFRVKAINLAGESGAAIIKEPVTVQEIMQRPKICVPRHLRQTLVKKVGETINIMIPFQGKPRPKISWMKDGQTLDSKDVGIRNSS
TDTILFIRKAELHHSGAYEVTLQIENMTDTVAITIQIIDKPGPPQNIKLADVWGFNVALEWTPPQDDGNAQILGYTVQKADKKTMEWYTVYDH
YRRTNCVVSDLIMGNEYFFRVFSENLCGLSETAATTKNPAYIQKTGTTYKPPSYKEHDFSEPPKFTHPLVNRSVIAGYNTTLSCAVRGIPKPK
IFWYKNKVDLSGDAKYRMFSKQGVLTLEIRKPTPLDGGFYTCKAVNERGEAEIECRLDVRVPQ (SEQ ID NO: 75)
>tr|F1NBZ9|F1NBZ9_CHICK Myosin-binding protein C, cardiac-type OS = Gallus gallus OX = 9031
GN = MYBPC3 PE = 4 SV = 3
MPEPAKKAVSAFTKKPKTTEVAAGSTAVFEAETEKTGIKVKWQRAGTEITDSEKYAIKAEGNKHSLTISNVGKDDEVTYAVIAGTSKVKFELK
VKEPEKSEPVAPAEASAPAASELPAPPVESNQNPEVPPAETQPEEPVDPIGLFVTRPQDGEVTVGGNITFTAKVAGESLLKKPSVKWFKGKWM
DLASKVGKHLQLHDNYDRNNKVYTFEMEIIEANMTFAGGYRCEVSTKDKFDSSNFNLIVNEAPVSGEMDIRAAFRRTSLAGGGRRMTSAFLST
EGLEESGELNFSALLKKSSSFLRTANRGDGKSDSQPDVDVWEILRKAPPSEYEKIAFQYGITDLRGMLKRLKRIKKEEKKSTAFLKKLDPAYQ
VDKGQKIKLMVEVANPDADVKWLKNGQEIQVSGSRYIFEAIGNKRILTINHCSLADDAAYECVVAEEKSFTELFVKEPPILITHPLEDQMVMV
GERVEFECEVSEEGATVKWEKDGVELTREETFKYRFKKDGKKQYLIINESTKEDSGHYTVKINGGVSVAELIVQEKKLEVYQSIADLTVKARD
QAVFKCEVSDENVKGIWLKNGKEVVPDERIKISHIGRIHKLTIEDVTPGDEADYSFIPQGFAYNLSAKLQFLEVKIDFVPREEPPKIHLDCLG
QSPDTIVVVAGNKLRLDVPISGDPTPTVIWQKVNKKGELVHQSNEDSLTPSENSSDLSTDSKLLFESEGRVRVEKHEDHCVFIIEGAEKEDEG
VYRVIVKNPVGEDKADITVKVIDVPDPPEAPKISNIGEDYCTVQWQPPTYDGGQPVLGYILERKKKKSYRWMRLNFDLLKELTYEAKRMIEGV
VYEMRIYAVNSIGMSRPSPASQPFMPIAPPSEPTHFTVEDVSDTTVALKWRPPERIGAGGLDGYIVEYCKDGSAEWTPALPGLTERTSALIKD
LVTGDKLYFRVKAINLAGESGAAIIKEPVTVQEIMQRPKIWLPRHLRQTLVKKVGETINIMIPFQGKPRPKISWMKDGQTLDSKDVGIRNSST
DTILFIRKAELHHSGAYEVTLQIENMIDTVAITIQIIDKPGPPQNIKLADVWGENVALEWTPPQDDGNAQILGYTVQKADKKTMEWYTVYDHY
RRTNCVVSDLIMGNEYFFRVFSENLCGLSETAATTKNPAYIQKTGTTYKPPSYKEHDFSEPPKFTHPLVNRSVIAGYNTTLSCAVRGIPKPKI
FWYKNKVDLSGDAKYRMFSKOGVLTLEIRKPTPFDGGFYTCKAVNERGEAEIECRLDVRVPQ (SEQ ID NO: 76)
>NP_998735.1 troponin I, cardiac muscle [Gallus gallus]
MAEEEEPKPPPLRRKSSANYRGYAVEPHAKRQSKISASRKLQLKTLLLQRAKRELEREEQERAGEKQRHLGELCPPPELEGLGVAQLQELCRE
LHARIGRVDEERYDMGTRVSKNMAEMEELRRRVAGGRFVRPALRRVRLSADAMMAALLGSKHRVGTDLRAGLRQVRKDDAEKESREVGDWRKN
VDALSGMEGRKKKFEAPGGGQG (SEQ ID NO: 77)
>tr|A0A1D5PF28|A0A1D5PF28_CHICK Troponin T, cardiac muscle isoforms OS = Gallus gallus
OX = 9031 GN = TNNT2 PE = 4 SV = 1
MKQKHQEKGESKPKPKPFMPNLVPPKIPDGERLDFDDIHRKRMEKDLNELQALIEAHFESRKKEEEELISLKDRIEQRRAERAEQQRIRSERE
KERQARMAEERARKEEEEARKKAEEEARKKKAFSNMLHFGGYMQKSEKKGGKKQTEREKKKKILSERRKPLNIDHLSEDKLRDKAKELWQTIR
DLEAEKFDLQEKFKRQKYEINVLRNRVSDHQKV (SEQ ID NO: 78)
>sp|F1RRT2|MYL4_PIG Myosin light chain 4 OS = Sus scrofa OX = 9823 GN = MYL4 PE = 1 SV = 3
MPPKKPEPKKEAAKAAPAPAPAPAPAPAPPPEPAKEPTFDPKSIKIDFTADQIEEFKEAFSLFDRTPTGEMKITYGQCGDVLRALGQNPTNAE
VLRVLGKPKPEEMNAKMLDFETFLPILQHISRNKEQGTYEDFVEGLRVFDKESNGTVMGAELRHVLATLGEKMTEAEVEQLLAGQEDANGCIN
YEAFVKHIMSG (SEQ ID NO: 79)
>tr|Q910C5|Q910C5_CHICK Chick atrial myosin heavy chain OS = Gallus gallus OX = 9031 PE = 2
SV = 1
MEALLGAAAPFLRAPEGPRPTPAGDTRGLCFVPHPQLEFIRARVTARAGNGVTVTTEMGETLTVPEADVHPQNPPKFDRIEDMAMLTFLHEPA
VLYNLKERYASWMIYTYSGLFCVTVNPYKWLPVYNAEVVAAYRGKKRTEVPPHIFSISDNAYQNMLTDRENQSILITGESGAGKTVNTKRVIQ
YFASIAAIGHRKKEVANSSKGTLEDQIIQANPALEAFGNAKTVRNDNSSRFGKFIRIHFGATGKLASADIETYLLEKSRVIFQLKAERNYHIF
YQILSNKKPELLEMLLITNNPYDYSYVSQGEVTVASIDDSEELLATDSAFDVLGFTAEEKAGVYKLTGAIMHFGNMKFKQKQREEQAEPDGTE
DCDKSAYLMGLNSADLLKGLCHPRVKVGNEYVTKGQSVQQVYYSIGALAKAVYEKMFNWMVVRINNSLETKQPRQYFIGVLDIAGFEIFDENS
FEQLCINFTNEKLQQFFNHHMFVLEQEEYKKEGIEWEFIDFGMDLQACIDLIEKPMGIMSILEEECMFPKASDMTFKAKLFDNHLGKSANFGK
PRNVKGKSEAHFSLIHYAGTVDYNIIGWLEKNKDPLNETVVGLYQKSALKLLASLFSNYAGADAGGDGGKGKGAKKKGSSFQTVSALHRENLN
KLMANLKTTHPHFVRCLIPNERKEPGVMDNPLVMHQLRCNGVLEGIRICRKGFPNRILYGDFRQRYRIPNPTAIPEGQFIDSRKGAEKLLGSL
DIDHNQYKFGHTKVFFKAGLLGLLEEMRDERLSLIITRIQAQARGQLMRIEFKKILERRDALLVIQWNIRAFMGVKNWPWMKLYFKIKPLLKS
AETEKEMQTMKEEFGHLKEALEKSAARRKELEEKMVSMLQEKNDLQLQVQAEQDNLADAEERCDQLIKNKIQLEAKVKEMTERLEEEEEMNAE
LAAKKRKLEDECSELKKDIDDLELSLAKVEKEKHATENKVKNLTEEMAGLDENITKLTKEKKILQESHQQALDDLQAEEDKVNTLAKAKGKLE
QQVDDLESSLEQEKKIRMDLERAKRKLEGDLKLAQESIMDLENDKQQLEERLKKKDFELNTLNARIEDEQAISAQLQKKLKELQARIEELEEE
LEAERTGRAKVEKLRSELLQELEETSERLEEAGGATSVQLELNKKREAEFQKLRRDLEEATLQHEATAATLRKKHADSVAELSEQLDNLQRVK
QKLEKEKSELKLELDDVNSNTEQLIKAKTNLEKMCRTTEDQMNEHRSKLEEAQRTVTDLSTQRAKLQTENSELSRQLEEKEAFINQLTRGKLT
YTQQLEDLKRQLEEEAKAKNALAHALQSAQHDCDLLREQYEEEMEAKAELQRALSKANSEVAQWRTKYETDAIQRTEELEEAKKKLAQRLQEA
EEAVEAVNAKCSSLEKTKHRLQNEIEDLMADVERSNAAAAALDKKQRNFDKILSEWKQKFEESQTELEASQKEARSLSTELFKLKNAYEESLE
HLETFKRENKNLQEEILDLTEQLGASQKSIHELEKVRKQLDAEKLELQAALEEAEASLEHEEGKILRAQLEFNQVKADYERKLAEKDEEIEQS
KRNHLRVVDSLQTSLDAETRSRNEALRLKKKMEGDLNEMEIQLSHANRTAAEAQKQVKALQGYLKDTQLQLDDVVRANEDLKENIAIVERRNN
LLQSELEELRAMVEQSERARKLAEQELIEASERVQLLHSQNTSLINQKKKMEADISQLQTEVEEAIQECRNAEEKAKKAITDAAMMAEELKKE
QDTSAHLERMKKNMEQTVKDLQLRLDEAEQLALKGGKKQLQKLEVRVRELENELEAEQKRNAESIKGLRKSERRVKELSYQTEEDRKNMVRLQ
DLVDKLQLKVKAYKRQAEEAEEQANSNLAKFRKVQHELDEAEERADMAESQVNKLRARSRDIGAKKGLNEE (SEQ ID NO: 80)
>tr|Q8UWA0|Q8UWA0_CHICK Myosin heavy chain OS = Gallus gallus OX = 9031 GN = SSMHC PE = 2
SV = 1
MSMLDMSEFGEAAEYLRKSYTEQLKLQTIPFDGKKRAWIPDEKEAYIEVEIKESTGGKVTVETKDKQTRVVKEDELQAMNPPKEDMIEDMAML
THLNEASVLYNLKRRYSHWMIYTYSGLFCVTINPYKWLPVYTAPVVAAYKGKRRSEAPPHIYSIADNAYNDMLRNRENQSMLITGESGAGKTV
NTKRVIQYFAIVAALGDTPGKKVAALATKTGGTLEDQIIEANPAMEAFGNAKTIRNDNSSRFGKFIRIHFGPSGKLASADIDIYLLEKSRVIF
QQPKERSYHIYYQILSGKKPELQDMLLLSLNPYDYHFCSQGVTTVDNLDDGEELMATDHAMDILGFSNDEKYGSYKIVGAIMHFGNMKFKQKQ
REEQAEADGTESADKAAYLMGISSADLIKGLLHPRVKVGNEYVTKGQNVEQVVYAVGALAKATYDRMFKWLVTRINKTLDTKLARQFFIGVLD
IAGFEIFDFNSFEQLCINFTNEKLQQFFNHHMFVLEQEEYKKEGIEWVFIDFGLDLQACIDLIEKPLGILSILEEECMFPKASDMSFKAKLYD
NHLGKSPNFQKPRPDKKRKYEAHFELVHYAGVVPYNIIGWLDKNKDPLNETVVAVFQKSQNKLLASLYENYVGSSSEEPHKPGSKEKRKKAAS
FQTVSQLHKENLNKLMTNLRSTQPHFVRCIIPNETKTPGAMDAFLVLHQLRCNGVLEGIRICRKGFPNRILYADFKQRYRILNPAAIPDDKFV
DSRKATEKLLSSLELDHSQYKFGHTKVFFKAGLLGMLEEMRDERLAKILTMLQARIRGHLMRIEYQKIISRREALYTIQWNIRAFNAVKNWSW
MKLFFKIKPLLKSAQTEKEMSTLKEEFQKLKEALEKSEAKRKELEEKQVSMIQEKNDLALQLQAEQDNLADAEERCDLLIKSKIQLEAKVKEL
TERVEDEEEMNADLTAKKRKLEDECAELKKDIDDLEITLAKVEKEKHATENKVKNLIEEMAALDEIIAKLTKEKKALQEAHQQALDDLQAEED
KVNTLTKAKVKLEQQVDDLESSLEQEKKIRMDLERAKRKLEGDLKLTQESVMDLENDKQQLEEKLKKKDFEMSQLNSRIEDQQVTEAQLQKKI
KELQARIEELEEELEAERAARAKVEKQRAEVSRELEELSERLEEAGGATSAQLEMNKKREVEFLKLRRDLEEATLQHESTAAALRKKHADSVA
ELSEQIDNLQRVKQKLEKEKSEMKMEVDDLSSNIEYLTKNKANAEKLCRTYEDQLSEAKSKVDELQRQLTDVSTQRGRLQTENGELSRLLEEK
ESFINQLSRGKTSFTQTIEELKRQLEEETKSKNALAHALQASRHDCDLLREQYEEEVEAKSELQRNLSKANAEVAQWRTKYETDAIQRTEELE
EAKKKLAIRLQEAEEAVEAAHAKCSSLEKTKHRLQTEIEDLSVDLERANSACAALDKKQRNFDRILAEWKQKYEETQAELEASQKESRSLSTE
LFKLKNAYEESLDNLETLKRENKNLQEEIADLTDQISMSGKTIHELEKLKKALENEKSDIQAALEEAEGALEHEESKTLRIQLELNQIKADVD
RKLAEKDEEFENLRRNHQRAMDSMQATLDAEARAKNEAVRLRKKMEGDLNEMEIQLSHANRQAAEFQKLGRQLQAQIKDLQIELDDTQRQNDD
LKEQAAALERRNNLLLAEVEELRAALEQAERSRKLAEQELLEATERVNLLHSQNTGLINQKKKLETDISQLSSEVEDAVQECRNAEEKAKKAI
TDAAMMAEELKKEQDTSAHLERMKKNMEQTIKDLQMRLDEAEQIALKGGKKQIQKLEARVRELEGELDMEQKKMAEAQKGIRKYERRIKELSY
QTEEDRKNLTRMQDLIDKLQSKVKSYKRQFEEAEQQANSNLVKYRKVQHELDDAEERADIAETQVNKLRARTKEVITFKHE (SEQ ID NO:
81)
>sp|P79293|MYH7_PIG Myosin-7 OS = Sus scrofa OX = 9823 GN = MYH7 PE = 2 SV = 2
MVDAEMAAFGEAAPYLRKSEKERLEAQTRPFDLKKDVYVPDDKEEFVKAKILSREGGKVTAETEHGKTVTVKEDQVLQQNPPKFDKIEDMAML
TFLHEPAVLYNLKERYASWMIYTYSGLFCVTINPYKWLPVYNAEVVAAYRGKKRSEAPPHIFSISDNAYQYMLTDRENQSILITGESGAGKTV
NTKRVIQYFAVIAAIGDRSKKEQTPGKGTLEDQIIQANPALEAFGNAKTVRNDNSSRFGKFIRIHFGATGKLASADIETYLLEKSRVIFQLKA
ERDYHIFYQILSNKKPELLDMLLITNNPYDYAFISQGETTVASIDDAEELMATDNAFDVLGFTSEEKNSMYKLTGAIMHFGNMKFKLKQREEQ
AEPDGTEEADKSAYLMGLNSADLLKGLCHPRVKVGNEYVTKGQNVQQVMYATGALAKAVYEKMFNWMVTRINTTLETKQPRQYFIGVLDIAGF
EIFDFNSFEQLCINFTNEKLQQFFNHHMFVLEQEEYKKEGIEWEFIDFGMDLQACIDLIEKPMGIMSILEEECMFPKATDMTFKAKLYDNHLG
KSNNFQKPRNIKGRPEAHFALIHYAGTVDYNIIGWLQKNKDPLNETVVDLYKKSSLKLLSNLFANYAGADTPVEKGKGKAKKGSSFQTVSALH
RENLNKLMTNLRSTHPHFVRCIIPNETKSPGVIDNPLVMHQLRCNGVLEGIRICRKGFPNRILYGDFRQRYRILNPAAIPEGQFIDSRKGAEK
LLGSLDIDHNQYKFGHTKVFFKAGLLGLLEEMRDERLSRIITRIQAQSRGVLSRMEFKKLLERRDSLLIIQWNIRAFMSVKNWPWMKLYFKIK
PLLKSAETEKEMATMKEEFGRLKEALEKSEARRKELEEKMVSLLQEKNDLQLQVQAEQDNLADAEERCDQLIKNKIQLEAKVKEMTERLEDEE
EMNAELTAKKRKLEDECSELKRDIDDLELTLAKVEKEKHATENKVKNLTEEMAGLDEIIAKLTKEKKALQEAHQQALDDLQAEEDKVNTLTKA
KVKLEQHVDDLEGSLEQEKKVRMDLERAKRKLEGDLKLTQESIMDLENDKQQLDERLKKKDFELNALNARIEDEQALGSQLQKKLKELQARIE
ELEEELEAERTARAKVEKLRSDLSRELEEISERLEEAGGATSVQIEMNKKREAEFQKMRRDLEEATLQHEATAAALRKKHADSVAELGEQIDN
LQRVKQKLEKEKSEFKLELDDVTSNMEQIIKAKANLEKMCRTLEDQMNEHRSKAEETQRSVNDLTSQRAKLQTENGELSRQLDEKEALISQLT
RGKLTYTQQLEDLKRQLEEEVKAKNALAHALQSARHDCDLLREQYEEETEAKAELQRVLSKANSEVAQWRTKYETDAIQRTEELEEAKKKLAQ
RLQDAEEAVEAVNAKCSSLEKTKHRLQNEIEDLMVDVERSNAAAAALDKKQRNFDKILAEWKQKYEESQSELESSQKEARSLSTELFKLKNAY
EESLEHLETFKRENKNLQEEISDLTEQLGSSGKTIHELEKVRKQLEAEKLELQSALEEAEASLEHEEGKILRAQLEFNQIKAEMERKLAEKDE
EMEQAKRNHLRVVDSLQTSLDAETRSRNEALRVKKKMEGDLNEMEIQLSHANRMAAEAQKQVKSLQSLLKDTQIQLDDAVRANDDLKENIAIV
ERRNNLLQAELEELRAVVEQTERSRKLAEQELIETSERVQLLHSQNTSLINQKKKMEADLSQLQTEVEEAVQECRNAEEKAKKAITDAAMMAE
ELKKEQDTSAHLERMKKNMEQTIKDLQHRLDEAEQIALKGGKKQLQKLEARVRELENELEAEQKRNAESVKGMRKSERRIKELTYQTEEDRKN
LLRLQDLVDKLQLKVKAYKRQAEEAEEQANTNLSKFRKVQHELDEAEERADIAESQVNKLRAKSRDIGTKGLNEE (SEQ ID NO: 82)
>tr|E1BJ10|E1BJ10_BOVIN Myosin binding protein H like OS = Bos taurus OX = 9913 GN = MYBPHL
PE = 4 SV = 1
MEAATAPEAALRPTLKVKEASPADADGPQASPRRGTGSPLSQLLPPIEEHPKIWLPRALRQTYIRKVGDTVNLLIPFQGKPKPQAIWTRDGCA
LDTSRVSVRNGERDSILFIREAQRADSGRYQLSVQLGGLEATATIDILVIERPGPPQSIKLVDVWGSSATLEWTPPQDTGNAALLGYTVQKAD
TKSGLWFTVLERCRRASCTVPNLIVGNSYTFRVFAENQCGLSENAPITADLAHIQKAATVYRTEGFAQRDFSEAPKFTQPLADCTTVIGYDTQ
LFCCVRASPKPKIIWLKNKMDIQGNPKYRALTHLGICSLEIRKPGPFDGGIYTCKAVNPLGEASVDCRVDVKAPN (SEQ ID NO: 83)
>sp|P02540|DESM_PIG Desmin OS = Sus scrofa OX = 9823 GN = DES PE = 1 SV = 4
MSQAYSSSQRVSSYRRTFGGAPSFPLGSPLSSPVFPRAGFGTKGSSSSVTSRVYQVSRTSGGAGGLGPLRASRLGATRVPSSSYGAGELLDES
LADAVNQEFLTTRTNEKVELQELNDRFANYIEKVRFLEQQNAALAAEVNRLKGREPTRVAEIYEEELRELRRQVEVLTNQRARVDVERDNLLD
DLQRLKAKLQEEIQLKEEAENNLAAFRADVDAATLARIDLERRIESLNEEIAFLKKVHEEEIRELQAQLQEQQVQVEMDMSKPDLTAALRDIR
AQYETIAAKNISEAEEWYKSKVSDLTQAANKNNDALRQAKQEMMEYRHQIQSYTCEIDALKGTNDSLMRQMRELEDRFASEASGYQDNIARLE
EEIRHLKDEMARHLREYQDLLNVKMALDVEIATYRKLLEGEESRINLPIQTFSALNFRETSPEQRGSEVHTKKTVMIKTIETRDGEVVSEATQ
QQHEVL (SEQ ID NO: 84)
>tr|F1SFP9|F1SFP9_PIG Leiomodin 3 OS = Sus scrofa OX = 9823 GN = LMOD3 PE = 4 SV = 3
MSEHSRNSDQEEPFDREIDEDEILANLSPEELKELQSEMDVMAPDPRLPVGMIQKDQTDKPPTGNFDHKSLVDYMYWQKASRRMLEDERVPVT
FVPSEEKPQEQRKEIDKGNKNMSQYLKEKLNNEIAAHKRESKSSDNEQETNDDDEDNEDDEDDEEDDAEDEEDEGDESDEKTKGEEEGEVKEP
IRNGESNCQQVPNKAFEEQKDRPEAQEKFEKKISKLDPKKLALDTSFLKVSARPSGNQTDLDGSLRRVRQNDPDMKELNLNNIENIPKEMLLD
FVNAMKKNKHIKTFSLANVGADESVAFALANMLRENRSITTLNIESNFITGKGIVAIMRCLQFNETLTELRFHNQRHMLGHHAEMEISRLLKA
NTTLLKMGYHFELPGPRMVVTNLLTRNQDKQRQKRQEEQKQQQLKEQKKLIAMLENGLGLPPGMWEMLGGPMPDSQMQEFLQPPPPKSLHPQT
APFSRRNEVMAKPAQPPKYRTDPDSFRVVKLKRIQRKSRMPEAREPPEKTNLKDVIKTLKPVPRNRPPPLVEITPRDQLLNDIRHSNIAYLKP
VQLPKELA (SEQ ID NO: 85)
>sp|Q5KR49|TPM1_BOVIN Tropomyosin alpha-1 chain OS = Bos taurus OX = 9913 GN = TPM1 PE = 2
SV = 1
MDAIKKKMQMLKLDKENALDRAEQAEADKKAAEDRSKQLEDELVSLQKKLKATEDELDKYSEALKDAQEKLELAEKKATDAEADVASLNRRIQ
LVEEELDRAQERLATALQKLEEAEKAADESERGMKVIESRAQKDEEKMEIQEIQLKEAKHIAEDADRKYEEVARKLVIIESDLERAEERAELS
EGKCAELEEELKTVTNNLKSLEAQAEKYSQKEDKYEEEIKVLSDKLKEAETRAEFAERSVTKLEKSIDDLEDELYAQKLKYKAISEELDHALN
DMTSI (SEQ ID NO: 86)
>tr|F1NK75|F1NK75_CHICK Uncharacterized protein OS = Gallus gallus OX = 9031 GN = TPM4
PE = 3 SV = 2
MEAIKKKMQMLKLDKENAIDRAEQAETDKKAAEDKCKQVEDELVALQKKLKGTEDELDKYSEALKDAQEKLEQAEKKATDAEGEVAALNRRIQ
LVEEELDRAQERLATALQKLEEAEKAADESERGMKVIENRAMKDEEKMEIQEMQLKEAKHIAEEADRKYEEVARKLVILEGELERAEERAEVS
EVKCSDLEEELKNVTNNLKSLEAQSEKYSEKEDKYEEEIKILSDKLKEAETRAEFAERTVAKLEKSIDDLEDELYAQKLKYKAISEELDHALN
DMTSL (SEQ ID NO: 87)
>tr|H9L074|H9L074_CHICK Uncharacterized protein OS = Gallus gallus OX = 9031 GN = TPM3
PE = 3 SV = 3
MEAIKKKMQMLKLDKENALDRAEQAEAEQKQAEERSKQLEDELAAMQKKLKGTEDELDKYSEALKDAQEKLELAEKKAADAEAEVASLNRRIQ
LVEEELDRAQERLATALQKLEEAEKAADESERGMKVIENRALKDEEKMELQEIQLKEAKHIAEEADRKYEEVARKLVIIEGDLERTEERAELA
ESKCSELEEELKNVTNNLKSLEAQAEKYSQKEDKYEEEIKILTDKLKEAETRAEFAERSVAKLEKTIDDLEDELYAQKLKYKAISEELDHALN
DMTSMARRALQESSFGEMGHLPWFPEPQRVYPVSGRVDFFSGMGGLTYGLKRT (SEQ ID NO: 88)
>sp|E1BTG2|LMOD2_CHICK Leiomodin-2 OS = Gallus gallus OX = 9031 GN = LMOD2 PE = 1 SV = 2
MSTFGYRRELSKYEDIDEDELLASLTEEELKELERELEDIEPDRNLPVGQRQKSLTEKTPTGTFSREALMAYWERETRKLLEKERLGACEKDS
EQEEDNSEDIQEECFTESNSEVSEEAYTEEDDEEEEEEEEEEEDEDDSDDEDEEKQNSAASERPVNCEDGRSSSHVRHKKCSNAKNSENLENG
HDGKDTENLSFKSSAIHPCGNPTVIEDALEKVRSNDPETTEVNLNNIENITSQMLIQFSQALRDNTVVKSFSLANTHADDNVAIAIAGMLKVN
QHITSLNIESNFITGKGVLAIMRALQHNKVLTELRFHNQRHIMGSQVEMDIVKLLKENTTLVKLGYHFDLAGPRMSMTSILTRNMDKQRQKRM
QEQRQQEYGCDGAINPKTKVLQKGTPRSSPYTSPKSSPWSSPKLPRKSAPAKSQPPAPAPPPPPPPPPPPPPPPPPVIPDKKAPTRNIAEVIK
QQESSRKALONGQKKKKGKKGKKHENSILKEIKDSLKSVSDRKSEEGSRPSTRPSTPQRSLHDNLMEAIRASSIKQLRRVEVPEALR (SEQ
ID NO: 89)
>sp|Q6QGC0|PDLI3_PIG PDZ and LIM domain protein 3 OS = Sus scrofa OX = 9823 GN = PDLIM3
PE = 2 SV = 1
MPQNVILPGPAPWGFRLSGGIDFNQPLIITRITPGSKAAAANLCPGDVILAIDGYGTESMTHADAQDRIKAAAHQLCLKIDRAET
RLWSPQVTEDGKAHPFKINLESEPQDVNYFEHKHNIRPKPFIIPGRSSGCSTPSGIDGGSGRSTPSSVSTLSTICPGDLKVAAKMAPNIPLEM
ELPGVKIVHAQFNTPMQLYSDDNIMETLQGQVSTALGETPSMSEPTTASVPPQSDVYRMLHDNRNEPTQPRQSGSFRVLQELVNDGPDDRPAG
TRSVRAPVTKIHGGAGGTQKMPLCDKCGSGIVGAVVKARDKYRHPECFVCADCNLNLKQKGYFFVEGELYCETHARARMRPPEGYDTVTLYPK
A (SEQ ID NO: 90)
>tr|Q3MHM1|Q3MHM1_BOVIN Calsequestrin OS = Bos taurus OX = 9913 GN = CASQ2 PE = 2 SV = 1
MTPSKSSKAPFTFLPSAFLGKKHFAQMKRAHLFVVGVYLLSSCRAEEGLNFPTYDGKDRVVSLTEKNFKQVLKKYDLLCLYYHEPLSSDKVVQ
KQFQLKEIVLELVAQVLEHKDIGFVMVDAKKEAKLAKKLGFDEEGSLYILKGDRTIEFDGEFAADVLVEFLLDLIEDPVEIINSKLEVQAFER
IEDHIKLIGFFKSEESEHYKAFEEAAEHFQPYIKFFATFDKGVAKKLSLKMNEVDFYEPFMDEPIAIPDKPYTEEELVEFVKEHQRPTLRRLR
PEDMFETWEDDINGIHIVAFAERSDPDGYEFLEILKQVARDNTDNPDLSIVWIDPDDFPLLVAYWEKTFKIDLFKPQIGVVNVTDADSVWMDI
PDDDDLPTAEELEDWIEDVLSGKINTEDDDNDDEEDEDDDDDDDNSDEEDNDDSDDDDE (SEQ ID NO: 91)
Smooth muscle tissue
>sp|P08023|ACTA_CHICK Actin, aortic smooth muscle OS = Gallus gallus OX = 9031 GN = ACTA2
PE = 3 SV = 2
MCEEEDSTALVCDNGSGLCKAGFAGDDAPRAVFPSIVGRPRHQGVMVGMGQKDSYVGDEAQSKRGILTLKYPIEHGIITNWDDMEKIWHHSFY
NELRVAPEEHPTLLTEAPLNPKANREKMTQIMFETFNVPAMYVAIQAVLSLYASGRTTGIVLDSGDGVTHNVPIYEGYALPHAIMRLDLAGRD
LTDYLMKILSERGYSFVTTAEREIVRDIKEKLCYVALDFENEMATAASSSSLEKSYELPDGQVITIGNERFRCPETLFQPSFIGMESAGIHET
TYNSIMKCDIDIRKDLYANNVLSGGTTMYPGIADRMQKEITALAPSTMKIKIIAPPERKYSVWIGGSILASLSTFQQMWISKQEYDEAGPSIV
HRKCF (SEQ ID NO: 92)
>sp|P63270|ACTH_CHICK Actin, gamma-enteric smooth muscle OS = Gallus gallus OX = 9031
GN = ACTG2 PE = 1 SV = 1
MCEEETTALVCDNGSGLCKAGFAGDDAPRAVFPSIVGRPRHQGVMVGMGQKDSYVGDEAQSKRGILTLKYPIEHGIITNWDDMEKIWHHSFYN
ELRVAPEEHPTLLTEAPLNPKANREKMTQIMFETFNVPAMYVAIQAVLSLYASGRTTGIVLDSGDGVTHNVPIYEGYALPHAIMRLDLAGRDL
TDYLMKILTERGYSFVTTAEREIVRDIKEKLCYVALDFENEMATAASSSSLEKSYELPDGQVITIGNERFRCPETLFQPSFIGMESAGIHETT
YNSIMKCDIDIRKDLYANNVLSGGTTMYPGIADRMQKEITALAPSTMKIKIIAPPERKYSVWIGGSILASLSTFQQMWISKPEYDEAGPSIVH
RKCF (SEQ ID NO: 93)
>sp|P24032|MLRN_CHICK Myosin regulatory light chain 2, smooth muscle minor isoform OS =
Gallus gallus OX = 9031 PE = 2 SV = 2
MSSKRAKTKTTKKRPQRATSNVFAMFDQSQIQEFKEAFNMIDQNRDGFIDKEDLHDMLASLGKNPTDEYLDAMMNEAPGPINFTMFLTMFGEK
LNGTDPEDVIRNAFACEDEEATGFIQEDYLRELLTTMGDRFTDEEVDELYREAPIDKKGNFNYIEFTRILKHGAKDKDD (SEQ ID NO:
94)
>sp|P10587|MYH11_CHICK Myosin-11 OS = Gallus gallus OX = 9031 GN = MYH11 PE = 1 SV = 4
MSQKPLSDDEKFLFVDKNFVNNPLAQADWSAKKLVWVPSEKHGFEAASIKEEKGDEVTVELQENGKKVTLSKDDIQKMNPPKFSKVEDMAELT
CLNEASVLHNLRERYFSGLIYTYSGLFCVVINPYKQLPIYSEKIIDMYKGKKRHEMPPHIYAIADTAYRSMLQDREDQSILCTGESGAGKTEN
TKKVIQYLAVVASSHKGKKDTSITQGPSFSYGELEKQLLQANPILEAFGNAKTVKNDNSSRFGKFIRINFDVTGYIVGANIETYLLEKSRAIR
QAKDERTFHIFYYLIAGASEQMRNDLLLEGFNNYTFLSNGHVPIPAQQDDEMFQETLEAMTIMGFTEEEQTSILRVVSSVLQLGNIVEKKERN
TDQASMPDNTAAQKVCHLMGINVTDFTRSILTPRIKVGRDVVQKAQTKEQADFAIEALAKAKFERLFRWILTRVNKALDKTKRQGASFLGILD
IAGFEIFEINSFEQLCINYTNEKLQQLFNHTMFILEQEEYQREGIEWNFIDFGLDLQPCIELIERPTNPPGVLALLDEECWFPKATDTSFVEK
LIQEQGNHAKFQKSKQLKDKTEFCILHYAGKVTYNASAWLTKNMDPLNDNVTSLLNQSSDKFVADLWKDVDRIVGLDQMAKMTESSLPSASKT
KKGMFRTVGQLYKEQLTKLMTTLRNTNPNFVRCIIPNHEKRAGKLDAHLVLEQLRCNGVLEGIRICRQGFPNRIVFQEFRQRYEILAANAIPK
GFMDGKQACILMIKALELDPNLYRIGQSKIFFRTGVLAHLEEERDLKITDVIIAFQAQCRGYLARKAFAKRQQQLTAMKVIQRNCAAYLKLRN
WQWWRLFTKVKPLLQVTRQEEEMQAKDEELQRTKERQQKAEAELKELEQKHTQLCEEKNLLQEKLQAETELYAEAEEMRVRLAAKKQELEEIL
HEMEARIEEEEERSQQLQAEKKKMQQQMLDLEEQLEEEEAARQKLQLEKVTADGKIKKMEDDILIMEDQNNKLTKERKLLEERVSDLTTNLAE
EEEKAKNLTKLKNKHESMISELEVRLKKEEKSRQELEKIKRKLEGESSDLHEQIAELQAQIAELKAQLAKKEEELQAALARLEDETSQKNNAL
KKIRELESHISDLQEDLESEKAARNKAEKQKRDLSEELEALKTELEDTLDTTATQQELRAKREQEVTVLKRALEEETRTHEAQVQEMRQKHTQ
AVEELTEQLEQFKRAKANLDKTKQTLEKDNADLANEIRSLSQAKQDVEHKKKKLEVQLQDLQSKYSDGERVRTELNEKVHKLQIEVENVTSLL
NEAESKNIKLTKDVATLGSQLQDTQELLQEETRQKLNVTTKLRQLEDDKNSLQEQLDEEVEAKQNLERHISTLTIQLSDSKKKLQEFTATVET
MEEGKKKLQREIESLTQQFEEKAASYDKLEKTKNRLQQELDDLVVDLDNQRQLVSNLEKKQKKFDQMLAEEKNISSKYADERDRAEAEAREKE
TKALSLARALEEALEAKEELERTNKMLKAEMEDLVSSKDDVGKNVHELEKSKRTLEQQVEEMKTQLEELEDELQAAEDAKLRLEVNMQAMKSQ
FERDLQARDEQNEEKRRQLLKQLHEHETELEDERKQRALAAAAKKKLEVDVKDLESQVDSANKAREEAIKQLRKLQAQMKDYQRDLDDARAAR
EEIFATARENEKKAKNLEAELIQLQEDLAAAERARKQADLEKEEMAEELASANSGRTSLQDEKRRLEARIAQLEEELDEEHSNIETMSDRMRK
AVQQAEQLNNELATERATAQKNENARQQLERQNKELRSKLQEMEGAVKSKFKSTIAALEAKIASLEEQLEQEAREKQAAAKTLRQKDKKLKDA
LLQVEDERKQAEQYKDQAEKGNLRLKQLKRQLEEAEEESQRINANRRKLQRELDEATESNDALGREVAALKSKLRRGNEPVSFAPPRRSGGRR
VIENATDGGEEEIDGRDGDENGKASE (SEQ ID NO: 95)
>sp|P02607|MYL6_CHICK Myosin light polypeptide 6 OS = Gallus gallus OX = 9031 GN = MYL6
PE = 1 SV = 3
MCDFSEEQTAEFKEAFQLFDRTGDGKILYSQCGDVMRALGQNPTNAEVMKVLGNPKSDEMNLKTLKFEQFLPMMQTIAKNKDQGCFEDYVEGL
RVFDKEGNGTVMGAEIRHVLVTLGEKMTEEEVEQLVAGHEDSNGCINYEELVRMVLSG (SEQ ID NO: 96)
>sp|P02542|DESM_CHICK Desmin OS = Gallus gallus OX = 9031 GN = DES PE = 1 SV = 1
QSYSSSQRVSSYRRTFGGGTSPVFPRASFGSRGSGSSVTSRVYQVSRTSAVPTLSTFRTTRVTPLRTYGSAYQGAGELLDFSLAD
AMNQEFLQTRTNEKVELQELNDRFANYIEKVRFLEQQNALMVAEVNRLRGKQPTRVAEMYEEELRELRRQVDALTGQRARVEVERDNLLDNLQ
KLKQKLQEEIQLKQEAENNLAAFRADVDAATLARIDLERRIESLQEEIAFLKKVHEEEIRELQAQLQEQHIQVEMDISKPDLTAALRDIRAQY
ESIAAKNIAEAEEWYKSKVSDLTQAANKNNDALRQAKQEMLEYRHQIQSYTCEIDALKGTNDSLMRQMREMEERFAGEAGGYQDTIARLEEEI
RHLKDEMARHLREYQDLLNVKMALDVEIATYRKLLEGEENRISIPMHQTFASALNFRETSPDQRGSEVHTKKTVMIKTIETRDGEVVSEATQQ
QHEVL (SEQ ID NO: 97)
>sp|P19352-2|TPM2_CHICK Isoform 2 of Tropomyosin beta chain OS = Gallus gallus OX = 9031
GN = TPM2
MEAIKKKMQMLKLDKENAIDRAEQAEADKKQAEDRCKQLEEEQQGLQKKLKGTEDEVEKYSESVKEAQEKLEQAEKKATDAEAEVASLNRRIQ
LVEEELDRAQERLATALQKLEEAEKAADESERGMKVIENRAMKDEEKMELQEMQLKEAKHIAEEADRKYEEVARKLVVLEGELERSEERAEVA
ESRVRQLEEELRTMDQSLKSLIASEEEYSTKEDKYEEEIKLLGEKLKEAETRAEFAERSVAKLEKTIDDLEESLASAKEENVGIHQVLDQTLL
ELNNL (SEQ ID NO: 98)
>sp|Q2QLE2|CAV2_PIG Caveolin-2 OS = Sus scrofa OX = 9823 GN = CAV2 PE = 3 SV = 1
MGLETEKADVQLFMDDDSYSRHSGVDYADPKKFVDPGTDRDPHRLNSNLKVGFEDVIAEPVSTHSFDKVWICSHALFEISKYVIYKFLTVFLA
IPLAFAAGILFATLSCLHIWIII PFVKTCLMVLPSVQTIWKSVTDVVIAPLCTSAGRSFSSVSLOLSHD (SEQ ID NO: 99)
>sp|Q90623|MYPT1_CHICK Protein phosphatase 1 regulatory subunit 12A OS = Gallus gallus OX =
9031 GN = PPP1R12A PE = 1 SV = 1
MKMADAKQKRNEQLKRWIGSETDLEPPVVKRKKTKVKFDDGAVFLAACSSGDTEEVLRLLERGADINYANVDGLTALHQACIDDNVDMVKFLV
ENGANINQPDNEGWIPLHAAASCGYLDIAEYLISQGAHVGAVNSEGDTPLDIAEEEAMEELLQNEVNRQGVDIEAARKEEERIMLRDARQWLN
SGHINDVRHAKSGGTALHVAAAKGYTEVLKLLIQARYDVNIKDYDGWTPLHAAAHWGKEEACRILVENLCDMEAVNKVGQTAFDVADEDILGY
LEELQKKQNLLHSEKREKKSPLIESTANLDNNQTQKTFKNKETLIMEQEKNASSIESLEHEKADEEEEGKKDESSCSSEEEEDDDSESEAETD
KAKTLANANTTSTQSASMTAPSVAGGQGTPTSPLKKFPTSTTKVSPKEEERKDESPASWRLGLRKTGSYGALAEITASKEAQKEKDSAGVIRS
ASSPRLSSSLDNKEKEKDGKGTRLAYVAPTIPRRLASTSDIDEKENRDSSASSIRSGSSYARRKWEEDVKKNSLNEGPTSLNTSYQRSGSFGR
RQDDLVSSNVPSTASTVTSSAGLQKTLPASANTTTKSTTGSTSAGVQSSTSNRLWAEDSTEKEKDSVPTAVTVPVAPSVVNAAATTTAMTTAT
SGTVSSTSEVRERRRSYLTPVRDEESESQRKARSRQARQSRRSTQGVTLTDLQEAEKTIGRSRSTRTREQENEEKEKEEKEKQDKEKQEEKKE
SETKDDDYRQRYSRTVEEPYHRYRPTSTSTSTSSTSSLSTSTSSLSSSSQLNRPNSLIGITSAYSRSGTKESEREGGKKEEEKEEDKSQPKSI
RERRRPREKRRSTGVSFWTQDSDENEQEHQSDSEEGTNKKETQSDSLSRYDTGSLSVSSGDRYDSAQGRSGSQSYLEDRKPYCSRLEKEDSTD
FKKLYEQILAENEKLKAQLHDTNMELTDLKLQLEKTTQRQERFADRSLLEMEKRVSGKSQYLLGGKKSSRKKDI (SEQ ID NO: 100)
>sp|P26932-2|CNN1_CHICK Isoform Beta of Calponin-1 OS = Gallus gallus OX = 9031 GN = CNN1
MSNANFNRGPAYGLSAEVKNKLAQKYDPQTERQLRVWIEGATGRRIGDNFMDGLKDGVILCELINKLQPGSVQKVNDPVQNWHKLENIGNFLR
AIKHYGVKPHDIFEANDLFENTNHTQVQSTLIALASQAKTKGNNVGLGVKYAEKQQRRFQPEKLREGRNIIGLQMGTNKFASQQGMTAYGTRR
HLYDPKLGTDQPLDQATISLQMGTNKGASQGMTVYGLPRQVYDPKYCDAPGLLGEDGLNHSFYNSQ (SEQ ID NO: 101)
>tr|Q5ZKU6|Q5ZKU6_CHICK Calponin OS = Gallus gallus OX = 9031 GN = CNN2 PE = 2 SV = 1
MSSSQFNKGPSYGLSAEVKNRLAQKYDPQKEAELRTWIESVTGRQIGADFQKGLKDGVILCELMNKLQPNSVRKINRSALNWHQLENLSNFIK
AMVSYGMNPVDLFEANDLFESGNLTQVQVSLLALAGMAKTKGLQSGVDIGVKYSERQQRNFDEAKMKAGQCVIGLQMGTNKCASQSGMTAYGT
RRHLYDPKNQILPPMDHSTISLQMGTNKCASQVGMTAPGTRRHIYDAKMGLEKCDNSSMSLQMGSNQGANQSGQVFGLGRQIYDPKYCPQGTP
GDAANAAGEPGADPPGYHYYHQEESC (SEQ ID NO: 102)
>tr|E1BSX2|E1BSX2_CHICK Calponin OS = Gallus gallus OX = 9031 GN = CNN3 PE = 3 SV = 3
MTHFNKGPSYGLSAEVKNKIALKYDPQIEEDLRNWIEEVTGLSIGANFQLGLKDGIILCELINKLQPGSVKKINQSKLNWHQLENIGNFIKAI
QVYGMKPHDIFEANDLFENGNMTQVQTTLVALAGLAKTKGFHTTIDIGVKYAEKQARSFDAGKLKAGQSVIGLQMGTNKCASQAGMTAYGTRR
HLYDPKMQTDKPFDQTTISLQMGTNKGASQAGMLAPGTRRDIYDQKHILQPVDNSTISLQMGTNKVASQKGMSVYGLGRQVYDPKYCAAPTEP
VIHNGSQGTGTNGSEISDSDYQAEYPDDYHGEYQDDYQRDYHGQYSDQGIDY (SEQ ID NO: 103)
>sp|P19966|TAGL_CHICK Transgelin OS = Gallus gallus OX = 9031 GN = TAGLN PE = 1 SV = 3
MANKGPAYGMSRDVQSKIEKKYDDELEDRIVEWIVAQCGSSVGRPDRGRLGFQVWLKNGIVLSQLVNSLYPDGSKPVKIPDSPPTMVFKQMEQ
IAQFLKAAEDYGVVKTDMFQTVDLFEAKDMAAVQRTLVALGSLAVTKNDGHYHGDPNWFMKKAQEHKREFSESQLKEGKNIIGLQMGTNKGAS
QAGMSYGRPRQIIS (SEQ ID NO: 104)
>tr|A0A1L1RTM1|A0A1L1RTM1_CHICK Caldesmon OS = Gallus gallus OX = 9031 GN = CALD1 PE = 4
SV = 1
RLSYQRNDDDEEEAARERRRRARQERLRQKEEGDVSGEVTEKSEVNAQNSVAEEETKRSTDDEAALLERLARREERRQKRLQEALERQKEFDP
TITDGSLSVPSRREVNNVEENETTGKEEKVETRQGRCEIEETETVTKSYQRNNWRQDGEEEGKKEEKDSEEEKPKEVPTEENQVDVAVEKSTD
KEEVVETKTLAVNAENDTNAMLEGEQSITDAADKEKEEAEKEREKLEAEEKERLKAEEEKKAAEEKQKAEEEKKAAEERERAKAEEEKRAAEE
RERAKAEEERKAAEERERAKAEEERKAAEERAKAEEERKAAEERAKAEEERKAAEERAKAEKERKAAEERERAKAEEEKRAAEEKARLEAEKL
KEKKKMEEKKAQEEKAQANLLRKQEEDKEAKVEAKKESLPEKLQPTSKKDQVKDNKDKEKAPKEEMKSVWDRKRGVPEQKAQNGERELTTPKL
KSTENAFGRSNLKGAANAEAGSEKLKEKQQEAAVELDELKKRREERRKILEEEEQKKKQEEAERKIREEEEKKRMKEEIERRRAEAAEKRQKV
PEDGVSEEKKPFKCFSPKGSSLKIEERAEFLNKSAQKSGMKPAHTTAVVSKIDSRLEQYTSAVVGNKAAKPAKPAASDLPVPAEGVRNIKSMW
EKGNVFSSPGGTGTPNKETAGLKVGVSSRINEWLTKTPEGNKSPAPKPSDLRPGDVSGKRNLWEKQSVEKPAASSSKVTATGKKSETNGLRQF
EKEP (SEQ ID NO: 105)
>tr|E1C2S1|E1C2S1_CHICK Talin-1 OS = Gallus gallus OX = 9031 GN = TLN1 PE = 4 SV = 2
MVALSLKISIGNVVKTMQFEPSTMVYDACRMIRERVPEAQMGQPNDFGLFLSDEDPKKGIWLEAGKALDYYMLRNGDTMEYKKKQRPLKIRML
DGTVKTVMVDDSKTVTDMLMTICARIGITNYDEYSLVREIMEEKKEEVTGTLKKDKTLLRDEKKMEKLKQKLHTDDELNWLDHGRTLREQGID
DNETLLLRRKFFYSDQNVDSRDPVQLNLLYVQARDDILNGSHPVSFDKACEFAGYQCQIQFGPHNEQKHKPGFLELKDELPKEYIKQKGERKI
FMAHKNCGNMSEIEAKVRYVKLARSLKTYGVSFFLVKEKMKGKNKLVPRLLGITKECVMRVDEKTKEVIQEWSLTNIKRWAASPKSFTLDFGD
YQDGYYSVQTTEGEQIAQLIAGYIDIILKKKKSKDHFGLEGDEESTMLEDSVSPKKSTVLQQQFNRVGKAELGSVALPAIMRTGAGGPENFQV
GTMPQAQMQITSGQMHRGHMPPLTSAQQALTGTINSSMQAVNAAQATLDDFETLPPLGQDAASKAWRKNKMDESKHEIHSQVDAITAGTASVV
NLTAGDPADTDYTAVGCAVTTISSNLTEMSKGVKLLAALMEDEGGNGRQLLQAAKNLASAVSDLLKTAQPASAEPRQNLLQAAGLVGQTSGEL
LQQIGESDTDPRFQDMLMQLAKAVASAAAALVLKAKNVAQKTEDSALQTQVIAAATQCALSTSQLVACTKVVAPTISSPVCQEQLIEAGKLVA
KSAEGCVEASKAATNDDQLLKQVGVAATAVTQALNDLLQHIKQHATGGQPIGRYDQATDTILNVTENIFSSMGDAGEMVRQARILAQATSDLV
NAIKADAEGETDLENSRKLLSAAKILADATAKMVEAAKGAAAHPDSEEQQQRLREAAEGLRMATNAAAQNAIKKKLVHKLEHAAKQAAASATQ
TIAAAQHAAASNKNPAAQQQLVQSCKVVADQIPMLVQGVRGSQSQPDSPSAQLALIAASQNFLQPGGKMVAAAKATVPTITDQASAMQLSQCA
KNLAAALAELRTAAQKAQEACGPLEIDSALGLVQSLERDLKEAKAAARDGKLKPLPGETMEKCAQDLGNSTKAVTSAIAHLLGEVAQGNENYT
GIAAREVAQALRSLSQAARGVAANSSDPQVQNAMLECASDVMDKANNLIEEARKAVAKPGDPDSQQRLVQVAKAVSQALNRCVNCLPGQRDVD
AAIRMVGEASKRLLSDSFPPSNKTFQEAQSQLNRAAAGLNQSANELVQASRGTPQDLAKSSGKFGQDFNEFLQAGVEMASLSPTKEDQAQVVS
NLKSISMSSSKLLLAAKALSADPTSPNLKSQLAAAARAVTDSINQLITMCTQQAPGQKECDNALRELETVKELLENPTQTVNDMSYFSCLDSV
MENSKVLGESMAGISQNAKNSKLPEFGESISAASKALCGLTEAAAQAAYLVGVSDPNSQAGQQGLVDPTQFARANQAIQMACQNLVDPACTQS
QVLSAATIVAKHTSALCNTCRLASSRTANPVAKRQFVQSAKEVANSTANLVKTIKALDGAFNEENRERCRAATAPLIEAVDNLTAFASNPEFA
TVPAQISPEGRRAMEPIVTSAKTMLESSAGLIQTARSLAVNPKDPPQWSVLAGHSRTVSDSIKKLITNMRDKAPGQRECDEAIDVLNRCMREV
DQASLAAISQQLAPREGISQEALHNQMITAVQEINNLIEPVASAARAEASQLGHKVSQMAQYFEPLILAAIGAASKTPNHQQQMNLLDQTKTL
AESALQMLYTAKEAGGNPKQAAHTQEALEEAVQMMKEAVEDLTTTLNEAASAAGVVGGMVDSITQAINQLDEGPMGEPEGTFVDYQTTMVKTA
KAIAVTVQEMVTKSTTNPDELGILANQLTNDYGQLAQQAKPAALTAENEEIGSHIKRRVQELGHGCAALVTKAGALQCSPSDAYTKKELIESA
RKVSEKVSHVLAALQAGNRGTQACITAASAVSGIIADLDTTIMFATAGTLNRENSETFADHREGILKTAKALVEDTKVLVQNATASQEKLAQA
AQSSVSTITRLAEVVKLGAASLGSEDPETQVVLINAVKDVAKALGDLIGATKAAAGKAGDDPAVYQLKNSAKVMVTNVTSLLKTVKAVEDEAT
KGTRALEATIEHIRQELAVFSSPVPPAQVSTPEDFIRMTKGITMATAKAVAAGNSCRQEDVIATANLSRRAIADMLRACKEAAYHPEVSADVR
QRALRFGKECADGYLELLEHVLVILQKPTHELKQQLAGYSKRVASSVTELIQAAEAMKGTEWVDPEDPTVIAENELLGAAAAIEAAAKKLEQL
KPRAKPKQADESLDFEEQILEAAKSIAAATSALVKAASAAQRELVAQGKVGVIPANAVDDGQWSQGLISAARMVAAATNNLCEAANAAVQGHA
SEEKLISSAKQVAASTAQLLVACKVKADHDSEAMKRLQAAGNAVKRASDNLVKAAQKAAAFQDHDETVVVKEKMVGGIAQIIAAQEEMLRKER
ELEEARKKLAMIRQQQYKFLPTELRDEEQN (SEQ ID NO: 106)
>tr|E1BXG1|E1BXG1_CHICK Profilin OS = Gallus gallus OX = 9031 GN = PFN3 PE = 3 SV = 2
MNCWNYYTDCILSDKYIDDVAIVGLSDNKYVWAAKPGGLLSAVSPREVDLITGQDRITFLTAGISIAGKKCIVIRDSLLVEGDNVMDIRSRGG
DSRSICIGKTPKALIFLMGNRGVHGGVLNLKIHDMIAGMTL (SEQ ID NO: 107)
>tr|E1C9A2|E1C9A2_CHICK Profilin OS = Gallus gallus OX = 9031 GN = PFN4 PE = 3 SV = 2
MIQLQALLKESLIRTKHVENAAAIGINEREVCASTSGFYVPPENAINLIYAFYKNLLQVRKEGLYFRQKHYECVRADEHSIYLKNAEGGLIVV
KTNALILIATYRVGMYPSVCVEAVEKLGKTNTCTCMAACQRFIGWLCSCKELQKCV (SEQ ID NO: 108)
>sp|O93256|K1C19_CHICK Keratin, type I cytoskeletal 19 OS = Gallus gallus OX = 9031
GN = KRT19 PE = 2 SV = 1
MATYSFRQTTSSVAGGPCGRSLRLGGGSFRAPSIHGGSGGRGVSVSSARFVSSGLGSGLGGGYGGAFSSSFSAGFGGGYGGGLGSGDGLLSGN
EKTTMQNLNDRLASYLDKVRALEEANSDLETKIREWYLKQGPGPARDYSPYYKAIEDLRDQILAATIDNSKVVLQIDNARLAADDFKTKFETE
QALRMSVEADINGLRRVLDELTLARTDLELQIENLKEELAYLKKNHEEEMSALGGQVASQVSVEVDSAPGIDLSKILADMRDQYEHMAEKNRK
DAEAWFHSKTEELNRELAVNTEQLQSSKSEVTDLRRTLQGLEIELQSQLSMKGALESTLADTEGRYGAQLAQIQDMIGSIEAQLAELRADMER
QNSEYKMLMDIKTRLEQEIATYRQLLEGQESQLFGSLSGSPDKRDKPADGK (SEQ ID NO: 109)
Skeletal and cardiac muscle tissue
>tr|B5X7T1|B5X7T1_SALSA Troponin C, slow skeletal and cardiac muscles OS = Salmo salar
OX = 8030 GN = TNNC1 PE = 2 SV = 1
MDDVYKAAVENLTEEQKNEFKAAFDIACQGAEDGCISTKELGKVMRMLGQNPTPEELQEMIDEVDEDGSGTVDFDEFLVMMVRCMKEESKGKS
EEELAELFRMFDKNGDGYIDLEELKTMLESTGEAITEDDIEELMKDGDKNNDGKIDYDEFLEFMKGVE (SEQ ID NO: 110)
>sp|P09860|TNNC1_CHICK Troponin C, slow skeletal and cardiac muscles OS = Gallus gallus
OX = 9031 GN = TNNC1 PE = 1 SV = 1
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKT
EEELSDLFRMFDKNADGYIDLEELKIMLQATGETITEDDIEELMKDGDKNNDGRIDYDEFLEFMKGVE (SEQ ID NO: 111)
>sp|P63315|TNNC1_BOVIN Troponin C, slow skeletal and cardiac muscles OS = Bos taurus
OX = 9913 GN = TNNC1 PE = 1 SV = 1
MDDIYKAAVEQLTEEQKNEFKAAFDIFVLGAEDGCISTKELGKVMRMLGQNPTPEELQEMIDEVDEDGSGTVDFDEFLVMMVRCMKDDSKGKS
EEELSDLFRMFDKNADGYIDLEELKIMLQATGETITEDDIEELMKDGDKNNDGRIDYDEFLEFMKGVE (SEQ ID NO: 112)
>sp|P14315-2|CAPZB_CHICK Isoform 2 of F-actin-capping protein subunit beta isoforms 1 and 2
OS = Gallus gallus OX = 9031 GN = CAPZB
MSDQQLDCALDLMRRLPPQQIEKNLSDLIDLVPSLCEDLLSSVDQPLKIARDKVVGKDYLLCDYNRDGDSYRSPWSNKYDPPLEDGAMPSARL
RKLEVEANNAFDQYRDLYFEGGVSSVYLWDLDHGFAGVILIKKAGDGSKKIKGCWDSIHVVEVQEKSSGRTAHYKLTSTVMLWLQTNKTGSGT
MNLGGSLTRQMEKDETVSDSSPHIANIGRLVEDMENKIRSTINEIYFGKTKDIVNGLRSVQTFADKSKQEALKNDLVEALKRKQQS (SEQ
ID NO: 113)
>tr|E1BMP3|E1BMP3_BOVIN Myosin light chain kinase 3 OS = Bos taurus OX = 9913 GN = MYLK3
PE = 4 SV = 2
MSGAPKESLGPQGLPGLGKACLTTMDKKLNMLNEKVDKLLHFQEDVTEKLQCVYRGMGHLEQGLHRLEASRGLGPAGADRSPPSDAQAGWPEV
LELVRAGRQDAAQQGARLEALFRMVMAVDKAIALVGAVLQNSKVVDFIMQGSVPWRKGSLADNKEQVEEKEAKPKHTLSTRGVQAELRGPWEE
SQKADLPEGTGSDLPTQTEAPPEQRDGISGPTQVRPEVEVQAPRASSKNPGIGLELSVVSERVSEAPTGQEAALSAGRGTSPSRPDPRPSVEG
MRLTPAPPAQAKAAHGGGETPPRISIHVQETDTPGELLVTRGGSLRTSPPAETPAAVPPGEQDPPGPRCCPQAPGTESGKPILRGASVRKRSC
DEGAKAKEKQGPGSELTMAPSRARRDKGADSGASGPQQDMNPGAGNPDPGKDCTAGGVGSAEAGSRTPPGAEASSLVLDDSPAPPAPFEHRVV
SVRETSTSAGYTVCQHEVLGGGRFGQVHRCTEKATGLSLAAKIIKVKSAKDREDVKNEINIMNQLSHVNLIQLYDAFESKNSFTLVMEYVDGG
ELFDRITEEKYHLTELDVVLFTKQICEGVHYLHQHYVLHLDLKPENILCVNQTGHQIKIIDFGLARRYKPREKLKVNFGTPEFLAPEVVNYEF
VSFPTDMWSVGVITYMLLSGLSPFLGETDAETMNFIVNCNWDFDADTFEGLSEEAKDFVSRLLVKEKSCRMSATQCLKHEWLNNLPAKASKSK
VHLKSQLLLQKYMAQRKWKKHFYVVTAANRLRKFPTCP (SEQ ID NO: 114)
>tr|B5X8Q3|B5X8Q3_SALSA Troponin C OS = Salmo salar OX = 8030 GN = TNNC1 PE = 2 SV = 1
MNDIYKAAVEQLTDEQKNEFKAAFDIFVQDAEDGCISTKELGKVMRMLGQNPTPEELQEMIDEVDEDGSGTVDFDEFLVMMVRCMKDDSKGKT
EEELADLFRMFDKNADGYIDLEELKVMLEATGEAITEDDIEELMKDGDKNNDGKIDYDEFLEFMKGVE (SEQ ID NO: 115)
>tr|A4GR69|A4GR69_PIG Telethonin OS = Sus scrofa OX = 9823 GN = TCAP PE = 4 SV = 1
MATSELSCQVSEENCERREAFWAEWKDLTLSTRPEEGCSLHEEDAERRETYHQQGQCQALVQRSPWLVMRMGILGRGLQEYQLPYQRVLPLPI
FTPAKVGAAKEEREETPIQLRELLALETALGGQCLDRQDVAEITKQLPPVVPVSKPGALRRSLSRSMSQEAQRG (SEQ ID NO: 116)
>sp|P04268|TPM1_CHICK Tropomyosin alpha-1 chain
MDAIKKKMQMLKLDKENALDRAEQAEADKKAAEERSKQLEDELVALQKKLKGTEDELDKYSESLKDAQEKLELADKKATDAESEV
ASLNRRIQLVEEELDRAQERLATALQKLEEAEKAADESERGMKVIENRAQKDEEKMEIQEIQLKEAKHIAEEADRKYEEVARKLVIIEGDLER
AEERAELSESKCAELEEELKTVTNNLKSLEAQAEKYSQKEDKYEEEIKVLTDKLKEAETRAEFAERSVTKLEKSIDDLEDELYAQKLKYKAIS
EELDHALNDMTSI (SEQ ID NO: 117)
>tr|Q05706|Q05706_CHICK Beta-tropomyosin OS = Gallus gallus OX = 9031 GN = BRT-1 PE = 2
SV = 1
MEAIKKKMQMLKLDKENAIDRAEQAEADKKQAEDRCKQLEEEQQGLQKKLKGTEDEVEKYSESVKEAQEKLEQAEKKATDAEAEVASLNRRIQ
LVEEELDRAQERLATALQKLEEAEKAADESERGMKVIENRAMKDEEKMELQEMQLKEAKHIAEEADRKYEEVARKLVVLEGELERSEERAEVA
ESKCGDLEEELKIVTNNLKSLEAQADKYSTKEDKYEEEIKLLGEKLKEAETRAEFAERSVAKLEKTIDDLEERSRQEAEKNRVLTNELRVILT
ELNN (SEQ ID NO: 118)
>sp|Q2KI43|CAV3_BOVIN Caveolin-3 OS = Bos taurus OX = 9913 GN = CAV3 PE = 2 SV = 1
MMAEEHTDLEAQIVKDIHFKEIDLVNRDPKNINEDIVKVDFEDVIAEPVGTYSFDGVWKVSYTTFTVSKYWCYRLLSTLLGVPLALLWGFLFA
CISFCHIWAVVPCIKSYLIEIQCISHIYSLCIRTFCNPLFAALGQVCSNIKVMLRKEV (SEQ ID NO: 119)
>sp|Q02173|MPSF_CHICK M-protein, striated muscle OS = Gallus gallus OX = 9031 PE = 2 SV = 1
MSSVAVPFYQRRHKHFDQSYRNIQTRYVLEEYAARKAASRQAAHYESTGLGKTTCRLCARRARSLAHEAMQESRKRTHEQKSHASDEKRIKFA
SELSSLEREIHMARHHAREQLDRLAIQRMVEENMALERHVVEEKISRAPEILVRLRSHTVWEKMSVRLCFTVQGFPSPVVQWYKNEELITPAS
DPAKYSVENKYGVHVLHINRADFDDSATYSAVATNIHGQASTNCAVVVRRFRESEEPHPAGIMPFHLPLSYDVCFTHFDVQFLEKFGVTFATE
GETLTLKCSVLVTPELKRLRPRAEWYRDDVLIKDSKWTKLYFGEGQAALSFTHINKDDEGLYTLRMVTKGGVNECSAFLFVRDADALIAGAPG
APMDVKCHDANRDYVIVTWKPPNTTSQNPVIGYFVDKCEVGLENWVQCNDAPVKICKYPVTGLYEGRSYIFRVRAVNSAGISRPSRVSEPVAA
LDPVDLERTQTVHVDEGRKIVISKDDLEGDIQIPGPPTNVHASEISKTYVVLSWDPPVPRGREPLTYFIEKSMVGSGSWQRVNAQVAVKSPRY
AVFDLAEGKPYVFRVLSANKHGISDPSEITEPIQPQDIVVVPSAPGRVVATRNTKTSVVVQWDKPKHEENLYGYYIDYSVVGSNQWEPANHKP
INYNRFVVHGLETGEQYIFRVKAVNAVGFSENSQESEAIKVQAALTCPSYPHGITLLNCDGHSMTLGWKAPKYSGGSPILGYYIDKREANHKN
WHEVNSSVISRTIYTVEDLTEDAFYEFKIAAANVVGIGHPSDPSEHFKCKAWTMPEPGPAYDLTVCEVRNTSLVLLWKAPVYEGKSPITGYLV
DYKEVDTEDWITANEKPTSHRYFKVTDLHQGHTYVFKVRAVNDAGVGKSSEISEPVFVEASPGTKEIFSGVDEEGNIYLGFECKEATDASHFL
WGKSYEEIEDSDKFKIETKGDHSKLYFKHPDKSDLGTYCISVSDTDGVSSSFVLDEEELERLMTLSNEIKNPTIPLKSELAYEVLDKGEVREW
IQAESLSPNSTYRFVINDKEVENGDRHKISCDHSNGIIEMVMDKFTIDNEGTYTVQIQDGKAKNQSSLVLIGDAFKAILAESELQRKEFLRKQ
GPHFSEFLYWEVTEECEVLLACKIANTKKETVFKWYRNGSGIDVDEAPDLQKGECHLTVPKLSRKDEGVYKATLSDDRGHDVSTLELSGKVYN
DIILALSRVSGKTASPLKILCTEEGIRLQCFLKYYNEEMKVTWSHRESKISSGEKMKIGGGEDVAWLQITEPTEKDKGNYTFEIFSDKESFKR
TLDLSGQAFDDALTEFQRLKAAAFAEKNRGKVIGGLPDVVTIMDGKTLNLTCTVFGNPDPEVVWFKNDKALELNEHYLVSLEQGKYASLTIKG
VTSEDSGKYSIYVKNKYGGETVDVTVSVYRHGEKIPEVNQGQLAKPRLIPPSSST (SEQ ID NO: 120)
>tr|E1BE25|E1BE25_BOVIN Filamin C OS = Bos taurus OX = 9913 GN = FLNC PE = 1 SV = 1
MMNNSGYSEAPGFGLGDEVDDMPSTEKDLAEDAPWKKIQQNTFTRWCNEHLKCVGKRLTDLQRDLSDGLRLIALLEVLSQKRMYR
KFHPRPNFRQMKLENVSVALEFLEREHIKLVSIDSKAIVDGNLKLILGLIWTLILHYSISMPMWEDEDDEDARKQTPKQRLLGWIQNKVPQLP
ITNFNRDWQDGKALGALVDNCAPGLCPDWEAWDPNQPVENAREAMQQADDWLGVPQVIAPEEIVDPNVDEHSVMTYLSQFPKAKLKPGAPVRS
KQLNPKKAIAYGPGIEPQGNTVLQPAHFTVQTVDAGIGEVLVYIEDPEGHTEEAKVVPNNDKNRTYAVSYVPKVAGLHKVTVLFAGQNIERSP
FEVNVGMALGDANKVSARGPGLEPVGNVANKPTYFDIYTAGAGTGDVAVVIVDPQGRRDTVEVALEDKGDSTFRCTYRPVMEGPHTVHVAFAG
APITRSPFPVHVAEACNPNACRASGRGLQPKGVRVKEVADFKVFTKGAGSGELKVTVKGPRGTEEPVKVREAGDGVFECEYYPVVPGKYVVTI
TWGGYAIPRSPFEVQVSPEAGIQKVRAWGPGLETGQVGKSADFVVEAIGTEVGTLGFSIEGPSQAKIECDDKGDGSCDVRYWPTEPGEYAVHV
ICDDEDIRDSPFIAHIQPAPPDCFPDKVKAFGPGLEPTGCIVDKPAEFTIDARAAGKGDLKLYAQDADGCPIDIKVIPNGDGTFRCSYVPTKP
IKHTIIVSWGGVNVPKSPFRVNVGEGSHPERVKVYGPGVEKTGLKANEPTYFTVDCSEAGQGDVSIGIKCAPGVVGPAEADIDFDIIKNDNDT
FTVKYTPPGAGRYTIMVLFANQEIPASPFHIKVDPSHDASKVKAEGPGLNRTGVEVGKPTHFTVLTKGAGKAKLDVHFAGAAKGEAVRDFEII
DNHDYSYTVKYTAVQQGNMAVTVTYGGDPVPKSPFVVNVAPPLDLSKVKVQGLNSKVAVGQEQAFSVNTRGAGGQGQLDVRMTSPSRRPIPCK
LEPGGGAETQAVRYMPPEEGPYKVDITYDGHPVPGSPFAVEGVLPPDPSKVCAYGPGLKGGLVGTPAPFSIDTKGAGTGGLGLTVEGPCEAKI
ECQDNGDGSCAVSYLPTEPGEYTINILFAEAHIPGSPFKATIRPVFDPSKVRASGPGLERGKAGEAATFTVDCSEAGEAELTIEILSDAGVKA
EVLIHNNADGTYHITYSPAFPGTYTITIKYGGHPVPKFPTRVHVQPAVDTSGVKVSGPGVEPHGVLREVTTEFTVDARSLTATGGNHVTARVL
NPSGAKTDTYVTDNGDGTYRVQYTAYEEGAHLVEVLYDDVAVPKSPFRVGVTEGCDPTRVRAFGPGLEGGLVNKANRFTVETRGAGTGGLGLA
IEGPSEAKMSCKDNKDGSCTVEYIPFTPGDYDVNITFGGRPIPGSPFRVPVKDVVDPGKVKCSGPGLGAGVRARVPQTFTVDCSQAGRAPLQV
AVLGPTGVAEPVEVRDNGDGTHTVHYTPATDGPYTVAVKYADQEVPRSLSSPFKIKVLPAHDASKVRASGPGLNASGIPASLPVEFTIDARDA
GEGLLTVQILDPEGKPKKANIRDNGDGTYTVSYLPDMSGRYTITIKYGGDEIPYSPFRIHALPTGDASKCLVTVSIGGHGLGACLGPRIQIGE
ETVITVDAKAAGKGKVTCTVSTPDGAELDVDVVENHDGTFDIYYTAPEPGKYVITIRFGGEHIPNSPFHVLACEAMPRVEEPPDVPQLHRPSA
YPTHWATEEPVVPAEPMESMLRPFNLVIPFTVQKGELTGEVRMPSGKTARPNITDNKDGTITVRYAPTEKGLHQMGIKYDGNHIPGSPLQFYV
DAINSRHVSAYGPGLSHGMVNKPATFTIVTKDAGEGGLSLAVEGPSKAEITCKDNKDGTCTVSYLPTAPGDYSIIVREDDKHIPGSPFTAKIT
GDDSMRTSQLNVGTSTDVSLKITESDLSQLTASIRAPSGNEEPCLLKRLPNRHIGISFTPKEVGEHVVSVRKSGKHVTNSPFKILVGPSEIGD
ASKVRVWGKGLSEGHTFQVAEFIVDTRNAGYGGLGLSIEGPSKVDINCEDMEDGTCKVTYCPTEPGTYIINIKFADKHVPGSPFTVKVTGEGR
MKESITRRRQAPSIATIGSTCDLNLKIPGNWFQMVSAQERLTRTFTRSSHTYTRTERTEISKTRGGETKREVRVEESTQVGGDPFPAVFGDEL
GRERLGSFGSITRQQEGEASSQDMTAQVTSPSGKTEAAEIVEGEDSAYSVRFVPQEMGPHTVTVKYRGQHVPGSPFQFTVGPLGEGGAHKVRA
GGTGLERGVAGVPAEFSIWTREAGAGGLSIAVEGPSKAEIAFEDRKDGSCGVSYVVQEPGDYEVSIKFNDEHIPDSPFVVPVASLSDDARRLT
VTSLQETGLKVNQPASFAVQLNGARGVIDARVHTPSGAVEECYVSELDSDKHTIRFIPHENGVHSIDVKENGAHIPGSPFKIRVGEQSQAGDP
GLVSAYGPGLEGGTTGVSSEFIVNTLNAGSGALSVTIDGPSKVQLDCRECPEGHVVTYTPMAPGNYLIAIKYGGPQHIVGSPFKAKVTGPRLS
GGHSLHETSTVLVETVTKSSSSRGSSYSSIPKFSSDASKVVTRGPGLSQAFVGQKNSFTVDCSKAGTNMMMVGVHGPKTPCEEVYVKHMGNRV
YNVTYTVKEKGDYILIVKWGDESVPGSPFKVNVP (SEQ ID NO: 121)
>tr|Q5ZLY3|Q5ZLY3_CHICK Tropomodulin 3 OS = Gallus gallus OX = 9031 GN = TMOD3 PE = 2 SV = 1
MTLPFRKDLDKYKDLDEDDILGKLSEEELKQLETVLDDLDPENALLPAGFRQKDQTAKKASGPFDRERLLAYLEKQALEHKDREDYVPFTKEK
KGKVFIPKQKPAQSYAEEKIALDPELEEALTSATDTELCDLAAILGMSNLITNNQFCDIVGSSNGVGKDSFSNIVKGEKMLPVFDEPPNPTNV
EETLQRIKDNDSRLVEVNLNNIKNIPIPTLKEFAKALETNTHVKNFSLAATRSNDPVAVALADMLRVNTKLKSLNIESNFITGVGILALVDAL
KDNETLTEIKIDNQRQQLGTLAEVEIAKMLEENTKILKFGYHFTQQGPRARAAAAITKNNDLVRKRRVEGDGQ (SEQ ID NO: 122)
>AAC14459.1 tropomodulin [Gallus gallus]
MSYRKELEKYRDLDEDKILGALTEEELRKLENELEELDPDNALLPAGLRQRDQTQKPPTGPFKREELMAHLEQQAKDIKDREDLVPFTGEKRG
KAWIPKQKPMDPVLESVTLEPELEEALANASDAELCDIAAILGMHTLMSNQQYYEALGSSTIVNKEGLNSVIKPTKYKPVPDEEPNSTDVEET
LKRIQNNDPDLEEVNLNNIMNIPVPTLKALAEALKTNTYVKKFSIVGTRSNDPVAFALAEMLKVNNTLKSLNVESNFISGSGILALVEALQSN
TSLIELRIDNQSQPLGNNVEMEIANMLEKNTTLLKFGYHFTQQGPRLRASNAMMNNNDLVRKRRLAELNGPIFPKCRTGV (SEQ ID NO:
123)
>tr|A0A287BJ41|A0A287BJ41_PIG Tropomodulin 1 OS = Sus scrofa OX = 9823 GN = TMOD1 PE = 1
SV = 1
MSYRRELEKYRDLDEDEILGALTEEELRTLENELDELDPDNALLPAGLRQKDQTTKAPTGPFKREELLDHLEKQAKEFKDREDLVPYTGEKRG
KVWVPKQKPMDPVLETVTLEPELEEALANASDAELCDIAAILGMHTLMSNQQYYQALGSSSIVNKEGLNSVIKPTQYKPVPDEEPNATDVEET
LERIKNNDPKLEEVNLNNIRNIPIPTLKAYAEALKENSYVKKFSIVGTRSNDPVAFALAEMLKVNKVLKTLNVESNFISGAGILRLVEALPYN
TSLVELKIDNQSQPLGNKVEMEIVSMLEKNATLLKFGYHFTQQGPRLRASNAMMNNNDLVRKRRLADLTGPIIPKCRSGI (SEQ ID NO:
124)
>tr|F1NXA5|F1NXA5_CHICK Coronin OS = Gallus gallus OX = 9031 GN = CORO6 PE = 3 SV = 3
MSRRVVRQSKFRHVFGQPVKADQMYEDIRVSKVTWDSSFCAVNPKFVAIIVEAGGGGAFMVLPLAKTGRVDKNHPLVTGHTAPVLDIDWCPHN
DNVIASASEDTTVMVWQIPDYVPVRSITEPVVTLEGHSKRVGIICWHPTARNVLLSAGCDNLVILWNVGTGEMLLALEDMHTDLIYNVGWNRN
GSLLVTTCKDKKVRVIDPRKQTVVAEITKPHDGARPIRAIFMADGKIFTTGFSKMSERQLGLWDLKNFEEPIALQEMDTSNGVLLPFYDPDTN
IVYLCGKGDSSIRYFEITDEAPYVHYLNTYSSKEPQRGMGFMPKRGLDVSKCEIARFFKLHERKCEPIVMTVPRKSDLFQDDLYPDTPGPEPA
LEADEWLSGKDAEPILISLRDGYVPVKNRELKVVKKNILDSKPPPGPRRSHSTSNTDISTPALDEVLEEIRVLKETVQAQEKRISALEHKLCQ
FTNGTD (SEQ ID NO: 125)
>XP_015136760.1 nebulette isoform X1 [Gallus gallus]
MYFSFMLAGLHRRKEFSLLFPPSIKMRVSVTQEFTEDENENGEEERVFLKPVIEDRNMELARKCSEIISDVHYKEEFEKSKGKCIFVPDTPQL
KHVKSVGAFISEVKYKGAAKKDLSNSLYQQMPATIDSAFAKELTQLQSKVLYKQKHDAEKGTSDYAHMKEPPDIKHAMEVNKYQSDVSYKRDV
QDTHRYTEVLNRPDIKMATEITKIISDAEYKKGRGEMNKEPAVLGRPDFEHAKGVSKLLSQVKYKEQFKKEMKSHQYNPLDSASFKQAQIAST
LASNVNYKKDYKESLHDPASDLPNLLYLNHALNISKMHSDVKYRENYEKSKGKSMLEFVDTPLYQVSKDVQKMQSEKIYRKDFEESLKGRPSL
DLDKTPEFLHIKQVTNLLKEKEYRKDLEEWMKGKGMTVFEDTPDLIRVKNAAQILNEKQYKKDLETEIKGKGMQVGPDTPEIRRAKKASEIAS
TKEYKKDLENEIKGKGMEVGMDTPDIQRAKKASEIVSQKEYRKDLETEIIGKGMQVGPFTPEIQRVKRASEIASQKMYKDEAEKMLCNYSAVP
DTPEMERIKSTQKNISSVFYKKVVGAGTAVKETPEIERVKKNQQNISSIKYKEETQHATPISDPPELRRIKENQKNISNVHYKEQLCRATPVS
VTPEIERVKRNQENVSMVHYREQPGKATAVSITPEIERVKKNQDNISSVKYSSDQRQMKGRRSVILDTPELRHVKETQNNISMVKYHEDFEKT
KGRGFTPVVDDPITERVRKNTQVVSDAAYKGVHPHIVEMDRRPGIIVDLKVWRTDPGSIFDIDPLEDNIQSRSLHMLSERASRYSKQYLHSTS
LGDYKSDGSDTNPTFSYCSEITRPSDEGAPVLPGAYQQSQSQGYGYMHQTSMSSMRSVHSQPHPAGLRTYRAMYDYSAQDEDEVSFRDGDYII
NVQPIDDGWMYGTVQRTGKTGMLPANYIEFVN (SEQ ID NO: 126)
>tr|F1MMX2|F1MMX2_BOVIN Nebulin-related-anchoring protein OS = Bos taurus OX = 9913
GN = NRAP PE = 4 SV = 1
MNVQACSRCGYGVYPAEKINCLDQIWHKACFHCEVCKMMLSVNNFVSYQKKPYCHAHNPKNNTFTSVYYTPLNLNVRKPPEAICG
IGGQEDGERFKSVFHWDMKSKDEAAAPNRQPQVDERAYWSGYREGDAWCPGALPDPEIVRMVEARKSLGEEYPEDYEQQRGKGSFPAMITPAY
QRAKIANQLASQVEYKRGHDERISRFSTVADTPELLRAKAGGQLQSDVRYTEAYEQQRGKGSFPAMITPAYQIAKRANELASDVRYHQQYQRE
MKGMAGPAAGAEGPLPKEYMDQYGQGYSEEYGEHRGKGSFPAMITPAYQNAKKANELASDIKYRQDFNKMKGAAHYHSLPAQDNLVLKRAQSV
NKLVSEVEYKKDLESSKGHSINYCETPQFRNVCKISKFTSDNKYKENYQNRMRGRYEGVGMDKRMLHALKVGSLASNIAYKADYKHDVVDYNY
PATLTPSYQTTVKLAPLKDVNYRQSIDKLKYSSVTNTPQIVQAKINAQQLSHVNYRADYEKNKLNYTLPQDVPQLVKARTNAELFSEVKYREG
WEKTKGKGFEMKLDAMSLLAAKASGELASNIKYKEEYEKAKGKVLGTTDSRLLHSLQVAKMSSEVEYKKGFEKSKTHFHLPMDMVNIRHAKKA
QALASDLDYRKRLHEYTVLPEDMKTLWAKKAYGLQSELQYKADLAWMKGVRWLTEGSLNLEQAKKAGQLVSEKNYRQRVDELKFTSVADSSQM
EHAKKSQELQSGVAYKAEHEQSVHQYSISKDEPLFLQARANAANLSEKLYKSSWENQKAKAFDLRLDSLAFLAAKAKRDLASEVKYKEDYEKS
RGKLIGAQGAQGDSQMSHSLQMSKLQSELEYKKGFEDTKSQCHVPLDMIHLVHARKAQHLATDIGYKTASHHFTALPTDMKVEWAKKAYGLQS
DNQYRADVKWMKGTGWVATGSLNVEQAKKAGELISEKKYRQHPDALKFTSIKDTPEMVQARISYTQAVDRLYREQGENLKHHYTQTTDLPEVL
LAKLNAMNISETRYKESWSKLRDGGYKLRLDAIPFQAAKASGEIISDYKYKEAFEKMKGQMLGSRSLEDDISLAHSVYASSLQSQVNYKKDFE
HSKAQFHLPLDMVTLVHAKKAQTLASDQDYRHPLPQYTSLAEDLRLSCAKRAHKLQSENLYRSDLNFMRGVACVIPGTLEIEGRKRASELISE
SKYRQHPQSLKYTAVTDTPSLTHAKLSNQITNERLYKAAGEDARHQYTMTLGLPEFVRAKTNAANLSDAKYKESWRNLHAQGYKLTIDALPFQ
AARVSGDIASDFLYRHDFVKERGRLIGAQSVSDDPRLQHCQRVGQLQSELQYRRQAAGSRAQCHLPMDMVPLVHARKAQALASDLDYRTQCHA
FTALPEDLRMAWAKKAHALQSELRYKSDLMGMKGTGWLALSSPQIESAKKAGELISETKYREKPDTIKFTTVVDSPDLVHAKNSYMHCNERLY
RSGDAESRHRYTLVPDHPDFTRARLNALNLSDKVYRHSWEQTRAGGYDFRLDAIPFQTARASREIASDFRYKEAFLRDRGLQIGYRSINDDPR
TKHFLSVGRLQSDNEYKKDFAKSRSQFHSRPDQPGFLQAKRSQQLASDVHYRQPLPQPTCDPEQLGLKHAQKAHRLQSDVKYKSDLNLTRGIG
WTPPGSYKVEMARRAAELANARGLGLQGAYGGPEAVEPRDDQSGFVNPDATEILHVKRRKAPLF (SEQ ID NO: 127)
>XP_003641574.1 nebulin-related-anchoring protein isoform X1 [Gallus gallus]
MNVQPCARCGYGVYPAEKINCIDQTWHKACFHCEVCKMMLTVNNFVSHEKKPYCQVHNPKNNAFTSIFETPINLNAKKLSEVVSE
VKYREESEQFKSAFQWDVRSRDIEAAYKAQHLSSQNAYYAAYEGGTSWYSGNMPDPQMVTVTQAQKNLNDLQYTEEYELRQGKGSFPAMITPG
YQVAKRATQLSSNVEYRRGHEERVSKFTSVVDTPDILHAKAGGQLASDLKYTEDFEEQRGKGSFPAMITPAYQIAKRANELASDVKYHQTYEK
EIKGKASHTAGTDVTFTRENVDQYGQDYMNEYEERRGKGSFPAMITPAYQNAKKANELASDIKYKKDLSKMKGAAHFHSLTAEDNLVLKQAQS
ANKLVSEVEYKKDLGNNRGYSVNYCDTPQFKNVSKISKYTSDIKYKETYQNQMKGHYMGIGMDKRMLHAMKVGNLASNIAYKSDYKHDGVDYN
YPATLTPSYQTTRKLVPLKDVNYRQSIDKMKYSSVASTPEIAQAKINAQQLSDLNYRAQYEKTKTNYTLPQDIPQLVKAKANAELYSEVKYKE
GWEKSKGQGFEMKLDSLPLLAAKASRDLASDIKYKEEYEKTKGKAIETKDSRLLHSLQVAKMSSEIAYKKDFEESKTHFHLPMDMVNLRHAKK
AQALASDLDYRKRLHEYTVVPEDLKTKWAKKAYGLQSDLQYREDLMWMKGVGCITEGSLNIQQAKKAGDLVSEKKYRQKVDALKFTSVADSSH
IKHAKKSQELQSDVAYRSGKEQFLHQYTITKDDPVFLLAKANAANISEKLYRSSWEKQKEKGFVLRLDALSFLTAKAKRDLASDIKYKEGYEK
MKGKLIGVKAVEEDSKMAHSLQMSKLQSDLEYKKAFEDTKNQFQVSMDMANLVHAKKAQNLASDKGYRTALHHYTVLPTDRKVAWAKWAYGLQ
SDNRYRADLNWMKGAGWIATGSLNVEQAKKAGELISEKKYRQHPYALKFTSIKDTPEMIQARISYNQAVDRLYKEHGESIKHQYTLTADLPEI
LRAKLNTMNISEIRYKESWQKMKDGGYQLRLDAIPFQAAKASGEIISDHKYKEAFEKMKGQMIGSCGLDGDIRIAHSVHASSLQSDVKYKQGF
EDTKTHFHLPLDMINLVHARKAQSLVSEQEYRQLLHQYTSLTDDLRLQCAKNAYKLQSENLYRSDMNFMRGVGCITPGALEIEGKKKASELIS
ESKYRQQPHSFKYTSVTDSPGLLHAKFSNMIANERLYKAAGEDIQHHYTPTLGLPEFTQARINAANLSDVKYRESWHNLCAQGYKLTMEALPF
QTARASREIASDYQYKHNFVMERGKHIGARSVLDDTRLLHCLHAAKLQSEQEYKKGSQGVWSQYYLPMDMVNLVHARKAQALASDQEYRKRLH
EFTALPEDLKMKWAKRAHMLQSEHRYKSDLNFMKGVGWMALRSPQIESVKKAGELISETKYRQKPESLKFTAVVDSPDLIHAKNSYLQCNDRL
YKAGDSEARHRYTLPPDHPDFIRARQNALNISDKVYKTSWEQTRASGYDFRIDAIPFQTAKASREIASDYKYREAFLRDRGQRIGFFSANDDA
HTRHVLRVGKLQSDNLYRSGYAQNRGHFQSHLNQPGFLHAKRSQQLASNVNYKQPLHQYTCDPEQLNVKHAKQAYKLQSDVKYKSDLNWLRGI
GWTPPGSYKVERARRAAELAYLREMGLQAASAQYGPEADQVGPEGSQQVTVNPDASEILQVKRRKMQLYK (SEQ ID NO: 128)
>sp|P08728|K1C19_BOVIN Keratin, type I cytoskeletal 19 OS = Bos taurus OX = 9913 GN = KRT19
PE = 2 SV = 1
MTSYSYRQSSSTSSFGGMGGGSMRFGAGGAFRAPSIHGGSGGRGVSVSSARFVSSSSGGYGGGYGGALATSDGLLAGNEKLTMQNLNDRLASY
LEKVRALEEANGDLEVKIRDWYQKQGPGPARDYSHYFKTIEDLRDQILGATIENSKIVLQIDNARLAADDFRTKFETEQALRMSVEADINGLR
RVLDELTLARTDLEMQIEGLKEELAYLKKNHEEEMSVLKGQVGGQVSVEVDSAPGIDLAKILSDMRSQYEVIAEKNRKDAEAWFISQTEELNR
EVAGHTEQLQISKTEVTDLRRTLQGLEIELQSQLSMKAALEGTLAETEARFGAQLAQIQALISGIEAQLSDVRADTERQNQEYQHLMDIKTRL
EQEIATYRNLLEGQDAYFNDLSLAKAL (SEQ ID NO: 129)
>tr|E1BF23|E1BF23_BOVIN Myomesin 2 OS = Bos taurus OX = 9913 GN = MYOM2 PE = 4 SV = 2
MSLVAVPFYQKRHKHFDQSYRNIQTRYLLDEYSAKKKRASAQSSLQRSVTRKSSSQRESSRAALGRTTCRLCAKRMSSALEEEAQ
ERKRRYQSMVAAYGEAKRERYLSELAQLEEDVHVARTHARDQLDRLALQHAVDDRLAWERHSFEERISRAPEILVRLRSHTVWERMSVKLCFT
VQGFPTPVVQWYKDGSLICQAGEPGKYRIDSKYGVHTLEINRADFDDTATYSAVATNVHGQVSTNAAVVVRRFRGDEEPFHSVGLPIGLPLSS
VIPYTHFDVQFIEKFGVTFRREGETLTLKCTLLVTPDLKRVQPRAEWYRDDVLLKESKWTKMFFGEGQASLSFSHLNKDDEGLYTLRIVSRGG
ITDHSAFLFVRDADPLVTGAPGAPMDVHCHDVNRDYVIVTWKPPNATKESPVIGYFIDRCEVGTDNWIQCNDAPVKICKYPVTGLFEGRSYIF
RVRAVNSAGISRPSRVSEAVAALDPVDLRRLQAVHLEGEKDIVVYQEELEGEVQIPGPPTNVHASETSRTYVVLSWDPPEPRGKEPLMYFIEK
SMVGSGSWQRVNAQTAVRSPRYAVFDLAEGKPYVFRVLSANKHGLSDPSEITAPIQAQDTIVVPSAPGRVLASRNTRTSVVVQWDRPKHEEDL
LGYYVDCSVAGSNVWEPCNHKPIGYNRFVVHGLTTGEQYIFRVKAVNAVGTSENSQESDVIKVQAALTVPSHPYGITLLNCDGHSMILGWKVP
KFSGGSPILGYYVDKREAHHKNWHEVNSSPLKERILTVEGLTEGSLYEFKIAAANMAGIGQPSDPSELFKCEAWTMPEPGPAYDLTFCEVRDT
SLVLLWKPPVYSGSSPVSGYFVDYKEEDSGEWLTVHETATPHRYLKVCDLHQGKTYVFRVRAVNASGVGRPSDTSEPVLVEARPGTKEVSAGV
DEEGNVYLSFDCPEVTDASHFTWCKSYEDIADDDRFKVETAGDHSKLYFKNLDKEDLGTYSVSVSDTDGVSSSFVLDEEELERLKALSNEIKN
PTIPLKSELAYEIFDKGQVRFWLQAEHLSPDSSYRFIINDREVADSETHRIKCDKSTGIIEMVMDRFTIDNEGTYTVQIQDGKAKSQSSLVLI
GDAFKAVLKEAEFQRKEFLRKQGPHFAEYLHWDVTEECEVRLVCKVANTKKETVFKWLKDDVLYETEKMPDLEKGVCELLIPKLSKKDHGEYK
ATLKDDRGQDVSTLEIAGKVYEDMILAMSRVCGASASPLKILCTPEGIRLQCFMKYFTEEMKVNWYHKDAKITSSEHMRIGGNEQMAWLQICE
PTEKDKGKYTFEILDGKKNHQRSLDLSGQAFDEAFAEFQQLKAAAFAEKNRGKVIGGLPDVVTIMEGKTLNLTCTVFGNPDPEVVWFKNDKDI
ELSDHFSVKVEQGKYVSMTIKGVTSEDSGKYSIHVKNKYGGEKIDVTVSVYKHGEEIPDVVLPQQAKPKLLPAAAPSPAH (SEQ ID NO:
130)
>tr|F1N0L9|F1N0L9_BOVIN Myopalladin OS = Bos taurus OX = 9913 GN = MYPN PE = 4 SV = 2
MQDDSLEASTSISQLLRESYLAETRHRGNNERSRAEPSSNPFHFGGSSGAAEGGGGQDDLPDLSAFLSQEELDESVNLARLAINY
DPLEKADEAQARKRFSSDQTKHSSISSFDPNFCQDNSQSPTNSKESLQETKRPQYSSEAQSKKVFLNKAADFIEELSSLFKAHSSKRIRPRAC
KNHKSKLESQNQVMQENSSSFPDLSERRERSSVPIPIPADTRDNEVNHALEQQEAKRREAEQAAGEAASGDTTPGSSPSSLYYEEPLGQPPRF
TQKLRSREVPEGTRVQLDCIVVGIPPPQVRWYCEGKELENSPDIHIIQAGNLHSLTIAEAFEEDTGRYSCFASNIYGTDSTSAEIYIEGVSSS
DSEGDPNKDEMNRIQKPNEVSSPPTTSAGIPSAVPQTQHVVVQPRVSTIQQCQSPTNYLQGLDGKPIIAAPVFTKMLQNLSASEGQLVVFECR
VKGAPSPKVEWYREGTLIEDSPDFRILQKKPRSMAEPEEICTLVIAEVFAEDSGCFTCTASNKYGTVSSIAQLDVRGNEDLRNNGSLHSANST
TNLALTEQQPSPPNPEPPVEQPPKPKLEGVLVNHNEPRSSSRIGLRVHFNLPEDDKGSEESSEGGVVTTHQTRPDSFQERFNGQSAKISEPSS
PVKEPPPVLAKPKLDSSQLQQLHNQVLLEQHHLQNPSPSSPKEFPFNMSVLNSTTVPAVTVSSKHAKAPPSQTFSLARPKHFFPSTSTTVASV
SPSSSPVFTLSSTPQAMQRTMSKESLLVTHPSAQTKSLGGASIQNEPLPTPAPTEPAQLPLTFSISSGNQFQPRCVSPAPVSPTGRIQNPVAF
LSSVLPSLPAIPPTNAMGLPKSAPSMPSQGLMKKNTKSSHPVSDDYIRETKNAVILDLGKKMNFSDVRSNQQEYKISSFEQRLMNEIEFRLER
TPVDESDDEIQHDEIPTGKCIAPIFDKRLKHFRVTEGSPVTFTCKIVGIPVPKVYWFKDGKQISKRNEHFKMKREGDGTCSLHINSTSSDDDG
NYTIMAANPQGRISCSGHLMVQGLPIRSRLTAAGQSHRGRSRVQERDKEPLQERFFRPHFLQAPGDMVAHEGRLCRLDCKVSGLPPPELTWLL
NGQPVLPDTSHKMLVRETGVHSLLIDPLTQRDAGTYTCIATNKTGQNSFSLELTVVAKEVKKAPVILEKLQNSGVPEGHPVRLECRVIGMPPP
VFYWKKDNETIPFTRERISMHQDTTGYVCLLIQPAKKSDAGWYTLSAKNEAGIVSCTARLDIYAQWHHQIPPPMTVRPSGSRYGSLTSKGLDI
FSAFSSMESTMVYSCSSRSVVESDEL (SEQ ID NO: 131)
>sp|Q5E9V3|MYOZ2_BOVIN Myozenin-2 OS = Bos taurus OX = 9913 GN = MYOZ2 PE = 2 SV = 1
MLSHNTMVKQRKQQASAIMKEIHGNDVDVMHLGKKVSIPRDIMLEELSHLSNRGARLFKMRQRRSDKYTFENFQYETKAQINHNIAMQNEKLD
GINLESGSQQAPFTPPNTPDPRSPPNPENIAPGYSGPLKEIPPERFNTTAVPKYYQSPWEQAISNDPELLEALYPKFFKPEGKAELPDYRSEN
RVATPFGGFEKASKMVKFKVPDFDLLLLTDPRFMAFANPLSGRRSFNRTPKGWISENIPIVITTEPTEDNTIPESEDL (SEQ ID NO:
132)
>tr|A0A1L1RJB3|A0A1L1RJB3_CHICK Uncharacterized protein OS = Gallus gallus OX = 9031
GN = MYOZ2 PE = 4 SV = 1
MLSHSAMVKERKQQASAIMDEIQRNVSPSLNLGKKVSTPRDIMVEELSTLSNRGARLFKRRQRRSDKYTYENYHYVANKPRNRGEPIHSVKMD
GLSVEGIPQHAPMTPPNTPDPRSPPHPDNIAPGYSGPLKEIPPEKFNTTSVPKYYQSPWIEAIRDDPELLEALYPKLFKPEAKPELPDYRSEN
RVATPFGGFERASKLVKFKVPDYNLLMLNDPRFMILANPLATRRSFNRTPKGWTSENIPVVFIQPSDSNNVPETEDL (SEQ ID NO:
133)
>tr|Q1AG08|Q1AG08_PIG Calsarcin 1 OS = Sus scrofa OX = 9823 GN = MYOZ2 PE = 2 SV = 1
MLSHNTMVKQRKQQASAIMKEIHGNDVDGMDLGKKVSIPRDIMLEELSHLSNRGARLFKMRQRRSDKYTFENFQYESKAQINHNIAMQNGKLD
GNNLESGSQQAPFTPPNTPDPRSPPNPENIAPGYSGPLKEIPPERFNTTAVPKYYQSPWEQAISNDPELLEALYPKLFKPEGKAELPDYRSEN
RVATPFGGFEKASKIVKFKVPDFELLLLTDPREMAFANPLSGRRSFNRTPKGWISENIPIVITTEPTEDTTIPESDDL (SEQ ID NO:
134)
>tr|A8E4L9|A8E4L9_BOVIN SYNC protein OS = Bos taurus OX = 9913 GN = SYNC PE = 2 SV = 1
MASPEPRRGGNGSAQAARATRPEVISPLQEENSASLYELGTWNPEVTLSLEGTLNLEDILYLGDTGDLDEALYVEETEPPEETLHIEETRMPD
EALYLEEPVRPEAALYVEEPVKLEGVLYVEEPVKTASPEQIVHGGDRVLSEAKSKPKESLQAGPSPSTEGSLSIEDLELLEGRFQQCIEAVAQ
LEEERDQLIHELVLLREPALQEVQQVHRDILAAYKQHAQAELERDGLREEIRLVKQKLFKVTKECVAYQYQLECRRQDVAQFADFREALTTRA
AQLSEELTQLREAYQKQKEQLRQQLEAPQSQRDGHFLQESRRLSAQFESLMAESRQGLEEEYEPQLLRLLERKEAAAKALQKTQAEIQEMKEA
LRPLQAEAQKLHLQNRNLEDQITLVRQKRDEEVQQYREQLEEMEERQRQLRSGVQLQQQKNKEMEQLRVSLAEELATYKAMLPKSLEQANAPT
SEAGGIETPSQGAV (SEQ ID NO: 135)
>tr|F1NM11|F1NM11_CHICK Syncoilin, intermediate filament protein OS = Gallus gallus OX = 9031
GN = SYNC PE = 4 SV = 4
MDAEGAGTPQAPEGTERPCLSLEELGEYFQECIEAVEQLEKERDALIEELTQLREPALQDIRHAHQEIQAACRLLAKVELERDNLRDEIRQIK
QKLFKVTKECVACQYQLESRRHDLSQHAAYRDELESQAGRLTGELSRLRESCEKEKEALRQRLEAPPCRQDPQYLQESRRLSAEFESLVTRSR
RGLEEHYEPQLLRLLERREAGTRALQELQGEVQGMKEALRPLQGEVSRLRLQNRSLEEQIVLVKQKRDEEVGQYREQVEELEDRLKELKNSVQ
LQQRKNQELEELRSSLHHELSIYKGCLEIYGHLCKSEEKPDQEC (SEQ ID NO: 136)
>tr|A0A287BKP7|A0A287BKP7_PIG Syncoilin, intermediate filament protein OS = Sus scrofa
OX = 9823 GN = SYNC PE = 3 SV = 1
MASPEPRRGGDGAAQAARETRAEATSPLQEGNSESLYQLGTWNPEVTLSLEGTLNLEDILYLGDPVDLDEALYVEETEKPEETLHIEETRLPD
EALYVEEPVKPEEMLYVEETVKPEEIVCVEQTMKPGETTSPEPITYGGETVPSEEKPNPEESLRAKPNPSTDGSLSLEDLELLEGRFQQCVQA
VAQLEEERDQLIHELVLLREPALQEVQQVHQDILAAYKLHAQAELERDGLREEIRLVKQKLFKVTKECVAYQYQLECRRQDVAQFADFREVLT
ARAAQLSEELAQLRDAYQKQKEQLRQQLEAPPSQRDGHFLQESRRLSARFENLMAESRQGLEEEYEPQLLRLLERKEAAAKALQKTQAEIQEM
KEALRPLQAEARQLHLQNRNLEDQITLVRQKRDEEVQQYREQLEEMEERQRQLKSGVQLQQQKNKEMEQLRISLAEELSTYKAMLPKSLERAS
APTSEAGGIETQSQGAV (SEQ ID NO: 137)
>tr|A4FV66|A4FV66_BOVIN Sarcoglycan alpha OS = Bos taurus OX = 9913 GN = SGCA PE = 2 SV = 1
MAAALIWISLLVGLLEGLGGTEAQQTTLHPLVGRVFVHTLDHESFLQRPEHVFSVSAPIPITYHAHLQGHPDLPRWLRYTQRSPYQPGFLYGT
ATPEDRGHQIIEVTAYNRDSFNTTQQMLVLLIGDPEGPLLPYQAEFLVRSHDVEEVLPSIPASRFLTALGGLWEPAELQLVNITSALDRGGRV
PLPIEGRKEGVYIKVGSASPFSTCLKMVVSPDSHARCAQGQPPLLSCYDTLAPHFRVDWCNVSLVDKSVPEPADEAPAPGDGILEHDPFFCPP
TEATNRDFLTDALVTLLVPLLVALLLTLLLAYVMCCRREGRLKRDLATSDIQMVHHRTIRGNTEELRQMASSREVPRPLSTLPMENVHTGERL
PPQVDSAQVPLILDQH (SEQ ID NO: 138)
>sp|Q0VCU7|SGCG_BOVIN Gamma-sarcoglycan OS = Bos taurus OX = 9913 GN = SGCG PE = 2 SV = 1
MVREQYTTITEGTHIERPENQAVYKIGIYGWRKRCLYLFVLLLLIVLLVNFALTIWILRVMWFSPVGMGHLHVTADGLHLEGESEFLFPLYVK
EIRSRVDSSLLLQSTQNVTMNARNTEGEVTGRLKVGPQMVEVQSQQFQIHSKDGKPLFTVDEEKVMVGTDKLRVTGPEGALFEHSVETPLVRP
DPPQDLRLESPTRSLSMDAPKGIHIQAPAGKIEALTQMDIVLQSSDGTVVLDAETVCLPELALGSQGPAGSSQGLYEVCVCPDGKLYLSVAGM
GTTCHEHSHLCL (SEQ ID NO: 139)
Skeletal and smooth muscle tissue
>sp|P12003|VINC_CHICK Metavinculin OS = Gallus gallus OX = 9031 GN = VCL PE = 1 SV = 4
MPVFHTRTIESILEPVAQQISHLVIMHEEGEVDGKAIPDLTAPVSAVQAAVSNLVRVGKETVQTTEDQILKRDMPPAFIKVENAC
TKLVRAAQMLQADPYSVPARDYLIDGSRGILSGTSDLLLTFDEAEVRKIIRVCKGILEYLTVAEVVETMEDLVTYTKNLGPGMTKMAKMIDER
QQELTHQEHRVMLVNSMNTVKELLPVLISAMKIFVTTKNTKSQGIEEALKNRNFTVEKMSAEINEIIRVLQLTSWDEDAWASKDTEAMKRALA
LIDSKMNQAKGWLRDPNAPPGDAGEQAIRQILDEAGKAGELCAGKERREILGTCKTLGQMTDQLADLRARGQGATPMAMQKAQQVSQGLDLLT
AKVENAARKLEAMTNSKQAIAKKIDAAQNWLADPNGGSEGEEHIRGIMSEARKVAELCEEPKERDDILRSLGEISALTAKLSDLRRHGKGDSP
EARALAKQIATSLQNLQSKTNRAVANTRPVKAAVHLEGKIEQAQRWIDNPTVDDRGVGQAAIRGLVAEGRRLANVMMGPYRQDLLAKCDRVDQ
LAAQLADLAARGEGESPQARAIAAQLQDSLKDLKARMQEAMTQEVSDVFSDTTTPIKLLAVAATAPSDTPNREEVFEERAANFENHAARLGAT
AEKAAAVGTANKTTVEGIQATVKSARELTPQVVSAARILLRNPGNQAAYEHFETMKNQWIDNVEKMTGLVDEAIDTKSLLDASEEAIKKDLDK
CKVAMANMQPQMLVAGATSIARRANRILLVAKREVENSEDPKFREAVKAASDELSKTISPMVMDAKAVAGNISDPGLQKSFLDSGYRILGAVA
KVREAFQPQEPDFPPPPPDLEHLHLTDELAPPKPPLPEGEVPPPRPPPPEEKDEEFPEQKAGEAINQPMMMAARQLHDEARKWSSKPVTVINE
AAEAGVDIDEEDDADVEFSLPSDIEDDYEPELLLMPTNQPVNQPILAAAQSLHREATKWSSKGNDIIAAAKRMALLMAEMSRLVRGGSGNKRA
LIQCAKDIAKASDEVTRLAKEVAKQCTDKRIRTNLLQVCERIPTISTQLKILSTVKATMLGRTNISDEESEQATEMLVHNAQNLMQSVKETVR
EAEAASIKIRTDAGFTLRWVRKTPWYQ (SEQ ID NO: 140)
>tr|E1BPV6|E1BPV6_BOVIN Smoothelin like 1 OS = Bos taurus OX = 9913 GN = SMTNL1 PE = 4 SV = 2
MEQKAGKSSEDGATVPPAAEAPEPAGSGGSAEEETAGPAESAARAGPATAGQQERAPAEDAVSAECQADGLGEVKAESQGEVELQQEDPGQGE
TAAAHGKTDRKDQTDSEPEGAEGDRETASASEEQSADEKEARLGSRETVDASREVQAEPKQASGAQEAEAGGGEAEGPQEEGGKTEEPQAEAR
EEAGAPAAQPDSEPGSPDEEQDQGAGAEAEDGAGGPPSSPEGWPESPTEEGGSASPEGLSPDTAASEELGPSASDSSPSDVPQSPTEPPPSEE
KKKEKAPERRVSAPTRPRGPRAQNRKAIVDKFGGAAAGPTALFRNTKAAGAAVGGVRNMLLEWCRAMTRSYEHVDIQNFSSSWGSGMAFCALI
HKFFPDAFDYAALDPAQRRHNFTLAFSTAEKLADCAQLLEVDDMVRLAVPDSKCVYTYIQELYRSLVQKGLVKTKKK (SEQ ID NO:
141)
Cardiac and smooth muscle tissue
>tr|F1NHA9|F1NHA9_CHICK PDZ and LIM domain protein 3 OS = Gallus gallus OX = 9031 GN = PDLIM3
PE = 4 SV = 1
MPQNVILPGPAPWGFRLSGGIDENQPLIITRITPGSKASTANLCPGDIIVAINGLSTENMTHNDAQERIKAAAHQLSLRIERAETKLWSPQVS
EDGKANPYKINLEAEPQEFKPIGTAHNRRAQPFVAAANIDDKRQVVSSSYNSPIGLYSSGNIQDALHGQLRSLIPNASQNDPAPTAVPQSDVY
RMLHSNQEEPSQPRQSGSFKVLQNLVSEEDGRPVGTRSVKAPVTKIPTGLPGAQKVPQCDKCGSGILGTVVKARDKYRHPECFVCSDCNLNLK
QKGYFFVEGQLYCETHARARMRPPEGYEAVTVYPKC (SEQ ID NO: 142)
Skeletal, cardiac, and smooth muscle tissue
>sp|Q04205|TENS_CHICK Tensin OS = Gallus gallus OX = 9031 GN = TNS PE = 1 SV = 2
MDFGSVMNQAATPCSPAVNYELPSPGQSITKQVDTPDATRSPRGGQAHCKASRSMSVTAAMESSCELDLVYITERIIAVSYPSTAEEQSFRSN
LREVAHMLKSKHGDNYVLFNLSERRHDISKLHPKVLDFGWPDLHTPALEKICSICKAMDTWLNAAAHNVVVLHNKGNRGRLGVVVAAYMHYSN
ISASADQALDREAMKRFYEDKVVPVGQPSQKRYIHYFSGLLSGSIKMNNKPLFLHHVIMHGIPNFESKGGCRPFLKIYQAMQPVYTSGIYNVQ
GDSQTGICITIEPGLLLKGDILLKCYHKKFRSPTRDVIFRVQFHTCAVHDLDIVFGKEDLDEAFRDERFPEYGKVEFVFSYGPEKIQGMEHLE
NGPSVSVDYNTSDPLIRWDSYENFNIQREDSAEGTWAEPALPGKHLEKEVGHTQGPLDGSLYAKVKKKDSLHGSIGAVNTARLPLSAAPNHVE
HTLSVSSDSGNSTASTKTDRTDEPGAPGAPTGHAVLSPEEKRDVDRLLVGFGLESAAPMHNHAPGPAPARLPAGPGRHVVPAQVHVNGAGTPL
LAERETDILDDELPNQDGHSVGSLGTLSSLDGTTTASEAGFHEAPRVGSLSSLPNGPASYNGAEKMLKEGLYEAEPLSNGAYPYSNQNTLMGH
HLRDPLAHLRPSRSAQEHLAGYPQRQPASASPAWLQPPVPQPYLYGYDLPSAHRSQSFPAVGTAKYEANLALPQAPARSTSSREAVQRGLNSW
QQQGGSRPPSQLHDGGLESHSPSLSSCSPQPSPLQPMPPHSHSMPEFPRAPSRREIEQSIEALDVLMLDLAPSVHKSQSVPSAATRQDKPAAM
LSSLSAQRLSGHYAQPTPQVVQPRSFGTSVGTDPLAKPYSPGPLVPAARSTAEPDYTVHEYRETYTPYSYQPVPEPRSYGSAPASILPLSASY
SPAGSQQLLVSSPPSPTAPAQSQLPHKGLESYEDLSRSGEEPLNLEGLVAHRVAGVQSREKSPEESTVPARRRTPSDSHYEKSSPEPGSPRSP
TVLSPEVVSTIAANPGGRPKEPHLHSYKEAFEEMESASPSSLTSGGVRSPPGLAKTPLSALGLKPHNPADILLHPVGELEGEAGADSEEEPRS
YVESVARTATTGRAGNLPAAQPVGLEVPARNGAFGNSFTVPSPVSTSSPIHSVDGASLRSYPSEGSPHGTVTPPHAVAETAYRSPMVSQTPSA
HSSYQTSSPSSFQAGTLGSPYASPDYPDGRGGFQPDPQARQQPQVSVVGVHALPGSPRTLHRTVATNTPPSPGFGRRAANPAVASVPGSPGLG
RHTVSPHAPPGSPSLARHQMAAVPPGSPMYGYSSPEERRPTLSRQSSASGYQPPSTPSFPVSPAYYPGTSTPHSSSPDSAAYRQGSPTPQPAL
PEKRRMSAGERSNSLPNYATVNGKASSPLSSGMSSPSSGSAVAFSHTLPDFSKFSMPDISPETRANVKFVQDTSKYWYKPDISREQAIALLKD
REPGAFIIRDSHSFRGAYGLAMKVASPPPTVMQQNKKGDITNELVRHFLIETSPRGVKLKGCPNEPNFGCLSALVYQHSIMPLALPCKLVIPD
RDPMEEKKDAASTTNSATDLLKQGAACNVLFINSVEMESLTGPQAISKAVAETLVADPTPTATIVHFKVSAQGITLTDNQRKLFFRRHYPLNT
VTFCDLDPQERKWTKTDGSGPAKLFGFVARKQGSTTDNVCHLFAELDPDQPAAAIVNFVSRVMLGSGQKR
(SEQ ID NO: 143)
>tr|A0A1D5PNS4|A0A1D5PNS4_CHICK Gelsolin OS = Gallus gallus OX = 9031 GN = GSN PE = 4 SV = 1
MSEVGEEQNGGRGAGLGGITAALGLVLIPIPIPILVPVPIPIPIPIPPPQGRKGQSRHRALAGAGPGWGGVRAVTAPGAARCARPRAPQLRPA
RPERQQQPVSMVEHAEFSKAGKEPGLQIWRIEKFDLVPVPKNLYGDFFTGDSYLVLNTIRQRSGNLQYDLHFWLGDESSQDERGAAAIFTVQM
DDYLQGKAVQHREVQGHESSTFLGYFKSGIKYKAGGVASGFRHVVPNEVTVQRLLQVKGRRTVRATEVPVSWESENTGDCFILDLGSNIYQWC
GSNSNRQERLKATVLAKGIRDNERNGRAKVFVSEEGAEREEMLQVLGPKPSLPQGASDDTKTDTANRKLAKLYKVSNGAGNMAVSLVADENPF
SQAALNTEDCFILDHGTDGKIFVWKGRSANSDERKAALKTATDFIEKMGYPKHTQVQVLPESGETPLFKQFFKNWRDKDQTEGLGEAYISGHV
AKIEKVPFDAATLHTSRAMAAQHGMEDDGSGKKQIWRIEGSEKVPVDPATYGQFYGGDSYIILYDYRHAGKQGQIIYTWQGAHSTQDEIATSA
FLTVQLDEELGGSPVQKRVVQGKEPPHLMSMFGGKPLIVYKGGTSREGGQTTPAQTRLFQVRSSTSGATRAVELDPVASQLNSNDAFVLKTPS
AAYLWVGRGSNSAELSGAQELLKVLGARPVQVSEGREPDNFWVALGGKAPYRTSPRLKDKKMDAHPPRLFACSNKSGRFTIEEVPGDLTQDDL
ATDDVMILDTWDQVFVWIGKDAQEEEKTEALKSAKRYIETDPASRDKRTPVTLVKQGLEPPTFSGWFLGWDDDYWSVDPLQRAMADVDV
(SEQ ID NO: 144)
>XP_025010159.1 dystroglycan isoform X1 [Gallus gallus]
MTVGCVPQPPFLGRTLLPVLLLAASARCHWPSEPAEVVRDWENQLEASMHSVLSDLRETVPAVVGIPDSSAVVGRFFRVSIPTDL
IASNGEAVQVSEAGKESLPSWLHWNAESSSLEGLPLDTDKGVHYISVTTLQPFPNGSYVPQAANVFSVEVHQEDHSEPQSVRAAAQEAGDAAP
FVCGAEEPVTILTVILDADLTKMTPKQRIELLNRMRSFSEVELHNMKLVPVVNNRLFDMSAFMAGPGNAKKVVENGALLSWKLGCSLSQNSVP
NISKVEAPAKEGTMSARLGYPVVGWHIANKKPHLPKRMRRQINATPTPLTAIGPPTTAAQEPPTRIVPTPTSPAIAPPTETTAPPVREPIPLP
RKPTVTIRTRGPIVQTPTLGPIQPTRLVEGTGTVSVPIRPTVPGYVEPTAVITPPTTTTKKPRVSTLKPATPSTTDSSTATTRRPTRRPKTPR
PTKPPSTTRSTISKLTTASPPTRVRTTASGVPRPWEPNEPPKLINHIDRVDAWEGTYFEVKIPSDTFYDKEDTTTDKLQLTLKLKEQQMIEEN
SWVQFNSTSQLMYGMPDRSHVGKHEYFMYATDKGGLFAVDAFEIHVHKRPHGDKSPVKFKARLEGDHSAVANDIHKKIMLVKKLALAFGDRNS
STITVQDIAKGSIVVEWINNTLPLEPCPREQIRTLSKKIADDSGGPSPAFSNILQPEFKPLNVSVVGSGSCRHIQFVPVTKDGRVISEATPTL
AAGKDPEKSSEDDVYLHTVIPAVVVAAILLVAGIIAMICYRKKRKGKLTIEDQATFIKKGVPIIFADELDDSKPPPSSSMPLILQEEKAPLPP
PEYPNQSMPETTPLNQDTIGEYTPLRDEDPNAPPYQPPPPFTAPMEGKGSRPKNMTPYRSPPPYVPP (SEQ ID NO: 145)
>sp|P12003-1|VINC_CHICK Isoform 1 of Vinculin OS = Gallus gallus OX = 9031 GN = VCL
MPVFHTRTIESILEPVAQQISHLVIMHEEGEVDGKAIPDLTAPVSAVQAAVSNLVRVGKETVQTTEDQILKRDMPPAFIKVENACTKLVRAAQ
MLQADPYSVPARDYLIDGSRGILSGTSDLLLTFDEAEVRKIIRVCKGILEYLTVAEVVETMEDLVTYTKNLGPGMTKMAKMIDERQQELTHQE
HRVMLVNSMNTVKELLPVLISAMKIFVTTKNTKSQGIEEALKNRNFTVEKMSAEINEIIRVLQLTSWDEDAWASKDTEAMKRALALIDSKMNQ
AKGWLRDPNAPPGDAGEQAIRQILDEAGKAGELCAGKERREILGTCKTLGQMTDQLADLRARGQGATPMAMQKAQQVSQGLDLLTAKVENAAR
KLEAMTNSKQAIAKKIDAAQNWLADPNGGSEGEEHIRGIMSEARKVAELCEEPKERDDILRSLGEISALTAKLSDLRRHGKGDSPEARALAKQ
IATSLQNLQSKTNRAVANTRPVKAAVHLEGKIEQAQRWIDNPTVDDRGVGQAAIRGLVAEGRRLANVMMGPYRQDLLAKCDRVDQLAAQLADL
AARGEGESPQARAIAAQLQDSLKDLKARMQEAMTQEVSDVFSDTTTPIKLLAVAATAPSDTPNREEVFEERAANFENHAARLGATAEKAAAVG
TANKTTVEGIQATVKSARELTPQVVSAARILLRNPGNQAAYEHFETMKNQWIDNVEKMTGLVDEAIDTKSLLDASEEAIKKDLDKCKVAMANM
QPQMLVAGATSIARRANRILLVAKREVENSEDPKFREAVKAASDELSKTISPMVMDAKAVAGNISDPGLQKSFLDSGYRILGAVAKVREAFQP
QEPDFPPPPPDLEHLHLTDELAPPKPPLPEGEVPPPRPPPPEEKDEEFPEQKAGEAINQPMMMAARQLHDEARKWSSKGNDIIAAAKRMALLM
AEMSRLVRGGSGNKRALIQCAKDIAKASDEVTRLAKEVAKQCTDKRIRTNLLQVCERIPTISTQLKILSTVKATMLGRTNISDEESEQATEML
VHNAQNLMQSVKETVREAEAASIKIRTDAGFTLRWVRKTPWYQ (SEQ ID NO: 146)
>tr|E1C8N4|E1C8N4_CHICK Supervillin OS = Gallus gallus OX = 9031 GN = SVIL PE = 4 SV = 3
MKRKERIARRLEGIENDTQPMLLQNCPGSVTHRLLEEDTPRYMRATDPYSHHLGRSNEEEEASDSSVEKQPRSSRYRTETTGGNTESLYSTGN
MDTHELESKAERIARYKAERRRQLAEKYGLSLDSDLDSDYSSRYSRARKDPDSVERKALRSERHDDENKDSGSLYLSRTEVKESKSVLSESRE
YSSREKDGIPAKEELSNEKSDKRADDDSAIRQASDPSATLDSSVSLTLSGRESSSCNEVPISPKQSPRESLSSPKRAASPIHLQNDQPLHSNI
RQSESRFEMSTSGSALSAGGDRERGPRRPRRYFTPGENRKTSERFKTQPITSAERKETDRSIMCAEIPTADDEEKLDDRAKLSVAAKRLLFRE
MEKSFEMKNIPKPPTRSSAVERRMRRLQDRSHTQPITSEEVVIAATLQASAHQKILAKEEIRAAKEAMGQDQSDEPDSSTLSLAEKMALENKL
SQPVSRAISTRSRGDIRHRRMNTRYQTQPVTLGEVEQVQNESGKLTPLSSTVSTSVSAMASALSALFAGDMHAKSRSDGSVSAATKDELRFHA
SAESSDSSGKRTQKSEQWQPSMEVLESKRASKKHEEEGKRFLAHEANEIRKYSSFEDETTHPVLEKTGDYHKEAAYSFLRKGSMELFSSQPLF
QPLERKEIDTIMQDHVEPTSELTSTPATRTLSQTTAAASCRLQELSEQLEGKFYKNSCEMFSAGENKIQTTEDATDSSSKTMSIKERLALLKK
SGEEDWKSRLSKKQEYAKVSATDRSAQMQEVEQLLKKRIADNQESQMTIEERKHLITAREEAWKSRGKGAANDSTQFTVAGRMVKKGLASPSA
LTPVASPYGNRQKSTTPVTKPLEEIEARPDMQLESDMKLDKLESFLGRLNNKVAGMQETVLTVTGKSVKEVMKLDDDETFSKFYRRVDFPSSS
VPLDLDEDFDAIFDPYAPKLISSVAEHKRAVRPMRRVQSSRNPLKMLAAREDILHEYTEQRLNVAFMESKRMKVEKMSANSNFSEVALAGLAS
KENFSNVSLRSVNLTEQNSNNSAVPYKKLMLLQIKGRRHVQTRLVEPRASSLNSGDCFLLLTPHLCFLWVGEFANVIEKAKASELATLIQTKR
ELGCRASYIQTIEEGINTHTHAAKDFWKLLGGQANYQSAGRPEEDEMYEAAIIETNCIYRLVEDKLIPEDDYWGKMPKCTLLQPKEVLVEDFG
SEVYVWHGKEVTLAQRKVAFQLAKHLWNGTFDYSNCDINPLDPGECNPLIPRKGQGRPDWAVFGRLTEHNETILFKEKFLDWTELKKLNEKNS
SESLHQKEESKSDSKPYDVMLMVPVPQTAVGTVLDGMNIGRGYGLVEGEDGRQFEIITASVDVWHILEFDYSRLPKQSIGQFHEGDTYVVKWK
YMVSTTVGSRQKGEQQVRAVGKEKCVYFFWQGRHSTVSEKGTSALMTVELDEERGAQVQVLQGKELPCFLQCFQGGMIVHAGRREEEEENAQS
DWRLYCVRGEVPNEGNLLEVACHCSSLRSRTSMIVLNINKALIYLWHGCKAQSHTKDVGRTAANKIKEQCPLEAGLHSSSKVTIHECDEGSEP
LGFWDALGRRDRKAYDCMLQDPGKFNFTPRLFSLSSSSGEFSATEYVYPSRDPTVINSMPFLQEDLYTAPQPALFLVDNHHEVYLWQGWWPVE
NKITGSARIRWATDRKCAMETVLQYCKGKNVKKPPKSYLIHAGLEPLTFTNMFPSWEHREDIAEITEMDADVSNQIILVEDVLAKLCKTVYPL
ADLLARPLPEGVDPLKLEIYLSDEDFEVALEMTREEYNALPSWKQVNLKKAKGLF (SEQ ID NO: 147)
>tr|Q5ZL50|Q5ZL50_CHICK Profilin OS = Gallus gallus OX = 9031 GN = PFN2 PE = 2 SV = 1
MAGWQSYVDNLMCDGCCQEAAIVGYCDAKYVWAATAGGIFQSITPVEIDMIVGKDREGFFTNGLTLGAKKCSVIRDSLYVDGDCT
MDIRTKSQGGEPTYNVAVGRAGRVLVFVMGKEGVHGGGLNKKAYSMAKYLRDSGF (SEQ ID NO: 148)
>sp|A5PJI6|CAVN4_BOVIN Caveolae-associated protein 4 OS = Bos taurus OX = 9913 GN = CAVIN4
PE = 2 SV = 2
MEHNGSASNADKIHQNRLSNVTEDEDQDAALTIVTVLDKVAAIVDSVQASQKRIEERHRVMENAIKSVQIDLLKFSQSHSNTGYVINKLFEKT
RKVSAHIKDVKARVEKQQTHVKKVEAKQEEIMKKNKFRVVIFQEEVQCPTSLSVVKDRSLTESPEEVDEIFDTPVDLSSDEEYFVEESRSARL
KKSGKERIDNIKKAFSKENMQKTRQNFDKKVNRIRTRIVTPERRERLRQSGERLRQSGERLKQSGERFKKSISNAAPSREAFKMRSLRKTKDR
AVAEGPEEVREMGVDIIARGEALGPISELYPEALSETDPEEASATHPPQEGGEVSTPEPLKVTFKPQVKVEDDESLLLDLKQ (SEQ ID
NO: 149)
>sp|Q62234|MYOM1 MOUSE Myomesin-1 OS = Mus musculus OX = 10090 GN = Myom1 PE = 1 SV = 2
MSLPFYQRSHQHYDLSYRNKDLRTTMSHYQQEKKRSAVYTHGSTAYSSRSLAARRQESEAFSQASATSYQQQASQTYSLGASSSSRHSQGSEV
SRKTASAYDYGYSHGLTDSSLLLEDYSSKLSPQTKRAKRSLLSGEETGSLPGNYLVPIYSGRQVHISGIRDSEEERIKEAAAYIAQKTLLASE
EAIAASKQSTASKQSATSKRTTSTLQREETFEKKSRNIAIREKAEELSLKKTLEETQTYHGKLNEDHLLHAPEFIIKPRSHTVWEKENVKLHC
SVAGWPEPRLTWYKNQVPINVHANPGKYIIESRYGMHTLEISKCDFEDTAQYRASAMNVQGELSAYASVVVKRYKGELDESLLRGGVSMPLSF
AVTPYGYASKFEIHFDDKFDVSFGREGETMSLGCRVVITPEIKHFQPEVQWYRNGAPVSPSKWVQPHWSGDRATLTFSHLNKEDEGLYTIRVR
MGEYYEQYSAYVFVRDADAEIEGAPAAPLDVVSLDANKDYIIISWKQPAVDGGSPILGYFIDKCEVGTDTWSQCNDTPVKFARFPVTGLIEGR
SYIFRVRAVNKTGIGLPSRVSEPVAALDPAEKARLKSHPSAPWTGQIIVTEEEPTEGVIPGPPTDLSVTEATRSYVVLSWKPPGQRGHEGIMY
FVEKCDVGAENWQRVNTELPVKSPRFALFDLVEGKSYRFRVRCSNSAGVGEPSETTEVTVVGDKLDIPKAPGKIIPSRNTDTSVVVSWEESRD
AKELVGYYIEASVVGSGKWEPCNNNPVKGSRFTCHGLTTAQSYIFRVRAVNAAGLSEYSQDSEAIEVKAAIGGGVSPDVWPQLSDTPGGLTDS
RGGMNGASPPTSQKDALLGSNPNKPSPPSSPSSRGQKEVSTVSESVQEPLSSPPQEAAPEEEQSQSEPPKKKKDPVAVPSAPYDITCLESFRD
SMVLGWKQPDTTGGAEITGYYVNYREVVGEVPGKWREANIKAVSDAAYKISNLKENTLYQFQVSAMNIAGLGAPSTVSECFKCEEWTIAVPGP
PHSVKLSEVRKNSLVLQWKPPVYSGRTPVTGYFVDLKEASAKDDQWRGLNEAAIVNKYLRVQGLKEGTSYVFRVRAVNQAGVGKPSDLAGPVV
AETRPGTKEVVVSVDDDGVISLNFECDQMTPKSEFVWSKDYVPTEDSPRLEVENKGDKTKMTFKDLGTDDLGTYSCDVTDTDGIASSYLIDEE
EMKRLLALSQEHKFPTVPTKSELAVEILEKGQVRFWMQAEKLSSNAKVSYIFNEKEIFEGPKYKMHIDRNTGIIEMFMEKLQDEDEGTYTFQI
QDGKATGHSTLVLIGDVYKKLQKEAEFQRQEWIRKQGPHFAEYLSWEVTGECNVLLKCKVANIKKETHIVWYKDEREISVDEKHDFKDGICTL
LITEFSKKDAGFYEVILKDDRGKDKSRLKLVDEAFQDLMTEVCKKIALSATDLKIQSTAEGIRLYSFVCYYLDDLKVNWSHNGTGIKYTDRVK
SGVTGEQIWLQINEPTPNDKGKYVMELFDGKTGHQKTVDLSGQAFDEAFAEFQRLKQAAIAEKNRARVLGGLPDVVTIQEGKALNLTCNVWGD
PPPEVSWLKNEKPLTSDDHCSLKFEAGKTAFFTISGVSTADSGKYGLVVKNKYGSETSDFTVSVFIPEEELRKGAMEPPKGNQKSK (SEQ
ID NO: 150)
>tr|F1MKE9|F1MKE9_BOVIN Spectrin beta chain OS = Bos taurus OX = 9913 GN = SPTB PE = 3
SV = 2
QPADMTSATEFENVGNQPPYSRINARWDGPDDELDNDNSSARLFERSRIKALADEREVVQKKTFTKWANSHLVHVSCRITDLYKDLRDGRMLI
KLLEVLSGEMLPKPTKGKMRIHCLENVDKALQFLREQRVHLENMGSHDIVDGNHRLVLGLIWTIILRFQIQDIVVQTQEGRETRSAKDALLLW
CQMKTAGYPHVNVTNFTSSWKDGLAFNALIHKHRPDLIDFDKLKDSNARHNLEHAFEVAERELGIIPLLDPEDVFTENPDEKSIITYVVAFYH
YFSKMKVLAVEGKRVGKVIDHAIETEKMIEKYSGLASDLLTWIEQTITILNSRKFANSLAGVQQQLQAFSTYRTVEKPPKFQEKGNLEVLLFT
IQSRMRANNQKVYTPHDGKLVSDINRAWESLEEAEYRRELALRDELIRQEKLEQLARRFDRKAAMRETWLNENQRLVAQDNFGYDLAAVEAAK
KKHEAIETDTAAYEERVRALEDLAQELERENYHDQKRIMARKDNILRLWDYLQELLQARRQRLEKTLDLQKLFQDMLHSIDWMDGIKAHLLSA
EFGKHLLEVEDLLQKHKLMEADIAIQGDKVKAITTATLQFTEGPGYQPCDPQVIQDRVSHLEQCFEELSNTAAGRKAQLEQSKQLCKFFWEMD
EAESWIKEKEQIYSSLDYGKDLTSVLILQRKHKAFEDELRGLDAHLDQIFREAEGMVARKQFGHERIEVRIKKVSDQWNELKDLAAFCKKNLQ
DTENFFQFQGDADDLKAWLQDAHRLLSGEDVGHDEGATRALGKKHKDFLEELEESRGVMEHLEQQAQAFPQGFQDSPDVINRLQALRDLYQQV
VAQADMRQQRLQDALDLYTVFGETDACELWMGEKGKWLAQMEIPDTLEDLEVVQHRFDILDQEMKTLMTQIDGVNLAANSLVESNHPRSAEVK
KYQDRLNTRWQDFQTMVSERREAVDSALRVHNYCVDCEETGKWIADKTKVVESTKDLGRDLAGVIAIQRKLSGLERDVAAIQARLGTLERESQ
QLMASHPELKEDIEQRQAYVEELWQGLMQALKSQEASLGEASQLQAFLQDLDAFQAWLSTTQKDVASKDMPESLPEAEQLLQQHAAIKDDIDR
HQESYQKVRVSGEKVTHGQTDPEYLLLGQRLDGLEKGWDALSRMWESRSQALTQCLGFQEFQKDAKQAEAILSNQEYTLAHLEPPDSLEAAEA
GIRKFEDFLLSMENNRDKVLSPVDSGNKLVAEGNLYSDKIKEKVQLIEDRHRKNNEKAQEASDLLKDNLELQNFLQNCQELTLWINDKLLTSQ
DVSYDEARNLHNKWLKHQAFVAELASHQGWLENIDAEGKQLMEEKPQFAVLVSQKLEALHQLWDELQANTQEMAQHLSAARSSDLRSQTQADL
NKWIRAMEDQLQSDDLGKDLTSVNRMLAKLKRVEDQVNVRKEELQELFAQMPSLGEEAGDEALSIEKRFLDLLEPLGRRKKQLESSRAKLQIS
RDLEDETLWVEERLPLAQSTDYGTNLQTVQLFMKKNQTLQNEILGHTPRVEDVLHRGQQLVAAAEIDCQDVEERLGKLQGSWDTLQEAAASRL
QHLREASEAQQYYLDAGEAEAWIGEQEFYVESDENPQDEESAIVMLKRHLRQQRAVDEYGRNIKQLAGRAQGLLSAGHPEGEQIIRLQGQVDK
QYAGLKDMAEERRRKLENMYHLFQLKREVDDLEQWITEKDTVASSSEMGQDFDHVTVLRDKFRDFARETGAIGQERVDNVNAIIERLIDAGHS
EAATIAEWKDGLNEMWADLLELIDTRMQLLAASYDLQRYFYTSSEILGLIGEKHRELPEDVGLDASTAESFHRTHTAFERELHLLGEQVQQFQ
DVAARLQTAYAGEKADIIQNKEQEVSAAWQALLDACAGRRTQLVDTADKFRFFSMARDLLSWMESIVRQIETQEKPRDVSSVELLMKYHQGIQ
AEIETRSKNFSTCLELGESLLQRQHQASEEIREKREQVVSRREEMQEKWEARWERLRMLLEVCQFFRDASVAEAWLIAQEPYLASRDYGHTVD
SVEKLIKRHEAFEKSAASWEGRFAALEKPTTLELKERQTPERTEEEPGLQEEEGETAGEAPRGPHQATTERTSPGEEERPWPQDLQPPPPPGP
HEDGQEEKSSTDERPATEPLFKVLDTPLSEGDEPTTLPAQRDRGHSVQMEGFLGRKHDLEGPNKKASNRSWNNLYCVLRNSELAFYKDAKNLA
LGVPYHGEEPLALRHAICEIAANYKKKKHVFKLRLSNGSEWLFHGKDEEEMLSWLQGVSTAINESQSIRVKAQSLPLPPITGPEASLGKKDKE
KRFSFFPKKK (SEQ ID NO: 151)
>tr|E1BIS6|E1BIS6_BOVIN Synemin OS = Bos taurus OX = 9913 GN = SYNM PE = 1 SV = 2
MLSWRLHTGPEKAELQELNARLYDYVCRVRELERENLLLEEELRSRRGQEGLWAEAEARCTEEARGLRRQLDELSWATALAQGERDALRRELR
ELQLLGEETRAARGRLDAELDSQRRELQEELAARAALEALLGRLQAERRGLDAARERDVRELRARAAGLTLRYRGRVAGPAAPPLRLRELHED
CALLVTEAWRETVQRYEDEVRELEEALRRGRESRREAEEETRLCAQEAEALRREVLELEQLRALLEEELLRVREASELQAEERQREIAYLEDE
KAALTLAMADRLRDYQDLVQVKTGLSLEVATYRALLEGESNPEILIVECIENMPQEFREKSYQYTTSVLQKENERNLFPRQKAPPASFRQSLA
PRSQTPVRSDARRDVLGSGYSSFTTAWQENAYQKTASGQTNFRTFSPTPGLLRNTEAQLKTFPERPRTEGTKAPPASSAKESAAAAEPYQERR
AEEAAAASEGTWSNERTVIWGKKTEAKATKEQERNRPGTIQTKREEKMFDSKEKASEERNLRWEELTKLDKEARKRESEQMREKAREKESLKE
ESVKGREIPIRLEASQDSTAKVAPQDLQTPLKEDAGDGTGRGVETREAGFTLGASDTTASLKGGSMTETIAENIVSSILKQFTHSPEAEASAE
SFPDTKVTYVDRKEVPGDRKAKTEIVVESKLTEEIDISDEAGLDYLLSKDAKEVELKGKSAERLIGDVIRLGLKGKEGRAKVVNVEIIEEPMS
YVSSEKEDEFSTPFEVEEIDDVSPSSRGLVEEREEAVYGESDVTCSGDAGQEARRPQESVTHVEEVTEAGDLEGEQSYFVSTPDEHPGAPERE
EGSVYGQIHIEEESTIRYSWQDEIVPGSRRRVRRDDVPGEKVVKPVDVPEVPLEGDTASAPWKEQTRSGEFHAKPIVIEKEIKIPHEFHTSIK
EGSKEPRHQLVEVIEQLEENLPERMKEELSALTREGQGGPAGVSFDVKKVQSAGSGAVTLVAEVNLSQTVDADQLDLEELSKDEAGEIEKAVE
SVVRDSLARRRSPGPGSPEAEAGGGFRRWATQELYSSSAGEADAGLASPPGPVSATVEVTSPTGFVHAHVLEDVSQSGGRVQIASPGMWRTEQ
VSAEGRAAQAVEVSAEGQASWAEGSAGTSRSARLITLGARQSPVSTEVIFPGPDAACPEAGGTEEPGPAELPTEPPRFGRHSPFGSQQFYAQR
EVIFQAPVSGVGKAGDSSQAEESAGTQTSVKHLQLGTREGLTEQTQLTAPLSGAVELGVREASVLMEAWSGDGTSIRHVTIGPQRHQATEPIV
PPPLEFSDSESSTHREGSADETLATSSYTVGRNILVTEKSTFQRAVSESPQEASAEDMSGNEVTSGVSRSFRHIQLGPAEAKTSEHIVFHGPI
SKTFALAGSVDSPEVSEVADSGRTLRHVALGPKETSFTFQMDVSNVEATSRWTQEARVLFPAGTEAEAPSVSEPGAWRDASSRNDLAAGVSFQ
GSAGDRHQAPGERGKEQAEFDKTVQLQRMVDQRSVISDEKKVALLYLDSQEEENEGHWF (SEQ ID NO: 152)
>tr|E1BF59|E1BF59_BOVIN Plectin OS = Bos taurus OX = 9913 GN = PLEC PE = 1 SV = 2
MVAGMFMPLDQLRAIYEVLFREGVMVAKKDRRPRSLHPHVPGVTNLQVMRAMASLRARGLVRETFAWRHFYWYLTNEGIAHLRQYLHLPPEIV
PASLQRVRRPVAMVMPARRTPHVQAVQGPLGCPPKRGPPPTEDPAREERCVYRRKEPEEGAPEPPVVPAATPGTLARQGLEPAPPTDERDRVQ
KKTFTKWVNKHLIKAQRHISDLYEDLRDGHNLISLLEVLSGDSLPREKGRMRFHKLQNVQIALDYLRHRQVKLVNIRNDDIADGNPKLTLGLI
WTIILHFQISDIQVSGQSEDMTAKEKLLLWSQRMVEGYQGLRCDNFTSSWRDGRLFNAIIHRHKPMLIDMNKVYRQTNLENLDQAFSVAERDL
GVTRLLDPEDVDVPQPDEKSIITYVSSLYDAMPRVPDVQDGVKANELQLRWQEYRELVLLLLQWIRAHTAAFEERRFPSSFEEIEILWCQFLK
FKETELPAKEADKNRSKGIYQSLEGAVQAGQLKVPPGYHPLDVEKEWGKLHVAILEREKQLRSEFERLERLQRIVSKLQMEAGLCEEQLNQAD
ALLQSQDVRLLAAGKAPQRAGEVERDLDKADGMIRLLENDVQALKDGRHPQGEQMYRRVYRLHERLVAIRTEYNLRLRGTPRHPELEDSTLRY
LQDLLAWVEENQRRLDGAEWGVDLPSVEAQLGSHRGLHQSVEEFRAKIERARTDEGQLSPATRGAYRDCLGRLDLQYAKLLNSSKARLRSLES
LHGFVAAATKELMWLSEKEEEEVGFDWSERNSNMAAKKEAYSALMRELELKEKKIKEIQSTGDRLLREDHPARPTVESFQAALQTQWSWMLQL
CCCIEAHLKENTAYFQFFSDVREAEEQLRKLQETLHRKYTCDRSITVTRLEDLLQDAQDEKDQLNEYRGHLSGLAKRAKAIVQLTPRNPTQPT
RGRVPLLAVCDYKQVEATVHKGDECQMLGPAQPFHWKVLSGSGSEAAVPSVCFLVPPPNQEALEAVARLEAQHQALVTLWHQLHTDMKSLLAW
QSLSRDVQLIRSWSLVTFRTLKPEEQRQALRSLELHYQAFLRDSQDAGGFGPEDRLQAEREYGSCSRHYQQLLQSLEQGKCGQCGXLDKEPAR
ECAQRIAEQQKAQAEVEGLGKGVARLSAEAEKVLALPEPSPAAPTLRSELELTLGKLEQVRSLSAIYLEKLKTISLVIRSTQGAEEALKAHEE
QLKEAQAVPAALPELEATKAAMKKLRAQAEAQQPVFDALRDELRGAQEVGERLQQRHGERDVEVERWRERVTQLLERWQAVLAQTDVRQRELE
QLGRQLRYYRESADPLGAWLQDARRRQEQIQAVPLADSQAVREQLRQEKALLEEIERHAEKVEECQRFAKQYINAIKDYELQLVTYKAQLEPV
ASPAKKPKVQSGSESVIQEYVDLRTRYSELSTLTSQYIRFISETLRRMEEEERLAEQQRAEERERLAEVEAALEKQRQLAEAHAQAKAQAERE
AQELQRRMQEEVARREEVVVDAQQQKRSIQEELQQLRQSSEAEIQAKARQVEAAERSRLRIEEEIRVVRLQLETTERQRGGAEGELQALRARA
EEAEAQKRQAQEEAERLRRQVQDETQRKRQAEAELGLRVKAEAEAAREKQRALQALEELRLQAEEAERRLRQAEAERARQVQVALETAQRSAQ
AELQSKHASFAEKTAQLERTLEEEHVTVVQLREEATRREQQQAEAERAREEAERELERWQLKANEALRLRLQAEEVAQQKSLAQAEAEKQKEA
AEREARRRGKAEEQAVRQRELAEQELERQRQLAEGTAQQRLAAEQELIRLRAETEQGEQQRQLLEEELARLQSEAAAATQKRQELEAELAKVR
AEMEVLLASKARAEEESRSSSEKSKQRLEAEAGRFRELAEEAARLRALAEEAKRQRQLAEEDAARQRAEAERVLSEKLAAISEATRLKTEAEI
ALKEKEAENERLRRLAEDEAFQRRRLEEQAAQHKADIEERLAQLRKASESELERQKGLVEDTLRQRRQVEEEILALKASFEKAAAGKAELELE
LGRIRGNAEDTLRSKEQAEQEAARQRQLAAEEERRRREAEERVQKSLAAEEEAARQRKAALEEVERLKAKVEEARRLRERAEQESARQLQLAQ
EAAQKRLQAEEKAHAFAVQQKEQELQQTLQQEQSMLERLRGEAEAARRAAEEAEEARERAEGEAAQSRQRVEEAERLKQAAEEQAQAQAQAQA
AAEKLRKEAEQEAARRAQAEQAALRQKQAADAEMEKHKKFAEQTLRQKAQVEQELTALRLKLEETDHQKSILDQELQRLKAEVTEAARQRSQV
EEELFSVRVQMEELGKLKARIEAENRALILRDKDNTQRLLQEEAEKMKQVAEEAARLSVAAQEAARLRQLAEEDLAQQRALAEKMLKEKMQAV
QEATRLKAEAELLQQQKELAQEQARRLQEDKEQMAQQLAQETQGFQRTLETERQRQLEMSAEAERLRLRVAEMSRAQARAEEDAQRFRKQAEE
ISAKLHRTELATQEKVTLVQTLETQRQQSDRDADRLREAIAELEREKDKLKKEAELLQLKSEEMQTVQQEQLLQETQALQQSFLSEKDSLLQR
ERFIEEEKAKLERLFQDEVAKAQKLREEQQRQQQQMQQEKQQLLASMEEARRRQREAEEGVRRKQEELQLLEQQRQQQEQLLAEENRRLRERL
EHLEEEHRAALAHSEEITAAQAAATRALPNGQDATDGPAAEPEHAFEGLRQKVPAQQLQEAGILSTEEVQRLVQGHTTVAELTQREDVRRYLQ
GHSSIAGLLLKPANEKLTIYAALRRQLLSPGTAVILLEAQAASGFLLDPVRNRRLTVNEAVKEGVVGPELHHKLLSAERAVTGYKDPYTGEQI
SLFQAMKKDLIVREHGIRLLEAQIATGGVIDPVHSHRVPVDVAYQRGYFDEEMNRVLQDPSDDTKGFFDPNTHENLTYLQLLERCVEDPETGL
RLLPLTDQAAKGGELVYTDSEARDVFEKATVSAPFGKFQGKTVTIWELINSEYFTAEQRRDLLRQFRTGKVTVEKIIKIVITVIEEHEQKGQL
CFQGLRALVPAAELLESGVIDWDLFRQLQLGERSVQEVAEVEAVRRALRGSGVIAGVWLEEARQKLSIYEALKKELLQPEAAVALLEAQAGTG
HVIDPATSARLTVDEAVRAGLVGPELHEKLLSAEKAVTGYKDPYSGQSVSLFQALKKGLIPREQGLRLLDAQLSTGGIVDPSKSHRLPLDVAC
ARGYLDKETSTALTAPRDDAKTYYNPRTWEPATYSQLQQQCRPDPLTGLSLLPLSEEAARARQQELYSEVQAREAFQKATVEVPVGSFQGRAV
TIWELINSEYFTAEQRQELLRQFRTGKVTVEKIIKIIITIVEEVETTRRERLSFSGLRAPVPASELLASGILSSSQFEQLKDGKTSVKDLSEL
DSVRTLLQGSGCLAGIYLEESKEKVTIYEAMRRGLLRPSTAILLLEAQAATGFLVDPVRNQRLYVHEAVKAGVVGPELHEKLLSAEKAVTGYK
DPYSGSTISLFQAMKKGLVVREHGIRLLEAQIATGGIIDPVHSHRVPVDVAYQRGYFDKEMNRVLEDPSDDTKGFFDPNTRENLTYRQLLERC
VEDPETGLRLLPLKGPEKAEVVETTRVYTEEETRRAFEETQIDIPGGGSHGGSTMSLWEVMQSDLIPEEQRAQLMADFQAGRVTKERMIIIII
EIIEKTEIVRQQNLASYDYVRRRLTAEDLHEARVISRESYSLLREGTRSLREVLEAESAWRYLYGTGCVAGVYLPGSRQTLTIYQALKKGLLS
AEVARLLLEAQAATGFLLDPVKGDRLTVDEAVRKGLVGPELHDRLLSAERAVTGYRDPYTEQTISLFQAMKKDLIPAEEALRLLDAQLATGGI
VDPRLGFHLPLEVAYQRGYLNKDTHDQLSEPSEVRSYIDPSTDERLSYTQLLRRCRRDEASGLFLLPLSDARKLTFRGLRKQITVEELVRSHV
MDEATAQRLQEGLTSIEEVSKNLQKFLEGTSSIAGVLVDATKERLSVYQAMKKGIIRPGTAFELLEAQAATGYVIDPIKGLKLTVEEAVRMGI
VGPEFKDKLLSAERAVTGYKDPYSGKLISLFQAMKKGLILKDHGIRLLEAQIATGGIIDPEESHRLPVDVAYQRGLFDEEMNEILTDPSDDTK
GFFDPNTEENLTYLQLMERCVTDPQTGLRLLPLKEKKRERKTSSKSSVRKRRVVIVDPETGKEMSVYEAYRKGLIDHQTYLELSEQECEWEEI
TISSSDGVVKSMIIDRRSGRQYDIDEAIAKSLIDRSALDQYRAGTLSITEFADMLSGNAGGFRSRSSSVGSSSSYPISPAVSRTQLASWSDPT
EETGPVAGILDTETLEKVSITEAMHRNLVDNITGQRLLEAQACTGGIIDPNTGERFPVTEAVNKGLVDKIMVDRINLAQKAFCGFEDPRTKTK
MSAAQALKKGWLYYEAGQRFLEVQYLTGGLIEPDTPGRVPLDEALQRGTVDARTAQKLRDVSAYSKYLTCPKTKLKISYKDALDRSMVEEGTG
LRLLEAATQSSKGYYSPYSVSGSGSTTGSRSGSRTGSRAGSRRGSFDATGSGFSMTFSSSSYSSSGYGRRYASGPTSSLGGPESTAA (SEQ
ID NO: 153)
>sp|Q63258-2|ITA7_RAT Isoform Alpha-7X1A of Integrin alpha-7 OS = Rattus norvegicus
OX = 10116 GN = Itga7
MARIPRCDFLGLPGICYLLSFLLAGLLLPRASAFNLDVMGAIRKEGEPGSLFGFSVALHRQLQPRPQSWLLVGAPQALALPGQQANRTGGLFA
CPLSLEETDCYRVDIDRGANVQKESKENQWLGVSVRSQGAGGKVVTCAHRYESRQRVDQVLETRDVIGRCFVLSQDLAIRDELDGGEWKFCEG
RPQGHEQFGFCQQGTAATFSPDSHYLIFGAPGTYNWKGLLFVTNIDSSDPDQLVYKTLDPADRLTGPAGDLTLNSYLGFSIDSGKGLMRSEEL
SFVAGAPRANHKGAVVILRKDSASRLIPEVVLSGERLTSGFGYSLAVTDLNSDGWADLIVGAPYFFERQEELGGAVYVYMNQGGHWADISPLR
LCGSPDSMFGISLAVLGDLNQDGFPDIAVGAPFDGDGKVFIYHGSSLGVVTKPSQVLEGEAVGIKSFGYSLSGGLDVDGNHYPDLLVGSLADT
AALFRARPVLHVSQEIFIDPRAIDLEQPNCADGRLVCVHVKVCFSYVAVPSSYSPIVVLDYVLDGDTDRRLRGQAPRVTFPGRGPDDLKHQSS
GTVSLKHQHDRVCGDTVFQLQENVKDKLRAIVVTLSYGLQTPRLRRQAPDQGLPLVAPILNAHQPSTQRTEIHFLKQGCGDDKICQSNLQLAQ
AQFCSRISDTEFQALPMDLDGTALFALSGQPFIGLELTVTNLPSDPARPQADGDDAHEAQLLATLPASLRYSGVRTLDSVEKPLCLSNENASH
VECELGNPMKRGTQVTFYLILSTSGITIETTELKVELLLATISEQDLHPVSVRAHVFIELPLSISGVATPQQLFFSGKVKGESAMRSERDVGS
KVKYEVTVSNQGQSLNTLGSAFLNIMWPHEIANGKWLLYPMRVELEGGQGPEKKGICSPRPNILHLDVDSRDRRRRELGQPEPQEPPEKVEPS
TSWWPVSSAEKRNVTLDCAQGTAKCVVFSCPLYSFDRAAVLHVWGRLWNSTFLEEYMSVKSLEVIVRANITVKSSIKNLLLRDASTVIPVMVY
LDPVAVVAEGVPWWVILLAVLAGLLVLALLVLLLWKCGFFRRNSPSSSFPANYHRAHLAVQPSAMEAGGPGTVGWDSSSGRSTLRPLYPSTTQ
(SEQ ID NO: 154)
>tr|A0A140T8D2|A0A140T8D2_BOVIN Integrin beta OS = Bos taurus OX = 9913 GN = ITGB1 PE = 1
SV = 1
MNLQLIFWIGLISSVCCVFGQADENRCLKANAKSCGECIQAGPNCGWCTNSTFLQEGMPTSARCDDLEALKKKGCHPNDIENPRGSKDIKKNK
NVTNRSKGTAEKLQPEDITQIQPQQLVLQLRSGEPQTFTLKFKRAEDYPIDLYYLMDLSYSMKDDLENVKSLGTDLMNEMRRITSDFRIGFGS
FVEKTVMPYISTTPAKLRNPCTNEQNCTSPFSYKNVLSLTDKGEVENELVGKQRISGNLDSPEGGFDAIMQVAVCGSLIGWRNVTRLLVFSTD
AGFHFAGDGKLGGIVLPNDGQCHLENDVYTMSHYYDYPSIAHLVQKLSENNIQTIFAVTEEFQPVYKELKNLIPKSAVGTLSANSSNVIQLII
DAYNSLSSEVILENSKLPEGVTINYKSYCKNGVNGTGENGRKCSNISIGDEVQFEISITANKCPNKNSETIKIKPLGFTEEVEIILQFICECE
CQGEGIPGSPKCHDGNGTFECGACRCNEGRVGRHCECSTDEVNSEDMDAYCRKENSSEICSNNGECVCGQCVCRKRDNTNEIYSGKFCECDNF
NCDRSNGLICGGNGVCKCRVCECNPNYTGSACDCSLDTTSCMAVNGQICNGRGVCECGACKCTDPKFQGPTCEMCQTCLGVCAEHKECVQCRA
FNKGEKKDTCAQECSHFNITKVENRDKLPQPGQVDPLSHCKEKDVDDCWFYFTYSVNGNNEATVHVVETPECPTGPDIIPIVAGVVAGIVLIG
LALLLIWKLLMIIHDRREFAKFEKEKMNAKWDTQENPIYKSPINNFKNPNYGRKAGL (SEQ ID NO: 155)
>sp|P07228|ITB1_CHICK Integrin beta-1 OS = Gallus gallus OX = 9031 GN = ITGB1 PE = 1 SV = 1
MAETNLTLLTWAGILCCLIWSGSAQQGGSDCIKANAKSCGECIQAGPNCGWCKKTDFLQEGEPTSARCDDLAALKSKGCPEQDIENPRGSKRV
LEDREVTNRKIGAAEKLKPEAITQIQPQKLVLQLRVGEPQTFSLKFKRAEDYPIDLYYLMDLSYSMKDDLENVKSLGTALMREMEKITSDFRI
GFGSFVEKTVMPYISTTPAKLRNPCTGDQNCTSPFSYKNVLSLTSEGNKFNELVGKQHISGNLDSPEGGFDAIMQVAVCGDQIGWRNVTRLLV
FSTDAGFHFAGDGKLGGIVLPNDGKCHLENNMYTMSHYYDYPSIAHLVQKLSENNIQTIFAVTEEFQAVYKELKNLIPKSAVGTLSSNSSNVI
QLIIDAYNSLSSEVILENSKLPKEVTISYKSYCKNGVNDTQEDGRKCSNISIGDEVRFEINVTANECPKKGQNETIKIKPLGFTEEVEIHLQF
ICDCLCQSEGEPNSPACHDGNGTFECGACRCNEGRIGRLCECSTDEVNSEDMDAYCRRENSTEICSNNGECICGQCVCKKRENTNEVYSGKYC
ECDNFNCDRSNGLICGGNGICKCRVCECFPNFTGSACDCSLDTTPCMAGNGQICNGRGTCECGTCNCTDPKFQGPTCEMCQTCLGVCAEHKDC
VQCRAFEKGEKKETCSQECMHFNMTRVESRGKLPQPVHPDPLSHCKEKDVGDCWFYFTYSVNSNGEASVHVVETPECPSGPDIIPIVAGVVAG
IVLIGLALLLIWKLLMIIHDRREFAKFEKEKMNAKWDTGENPIYKSAVTTVVNPKYEGK (SEQ ID NO: 156)
Sus scrofa (Pig)
>sp|Q9GLP0|ITB1_PIG Integrin beta-1 OS = Sus scrofa OX = 9823 GN = ITGB1 PE = 2 SV = 1
MNLQLIFWIGLISSVCYVFGQADENRCLKANAKSCGECIQAGPNCGWCTNSTFLQEGMPTSARCDDLEALRKKGCHPDDIENPRGSKNI
KKNKNVTNRSKGTAEKLQPEDITQIQPQQLVLQLRSGEPQTFTLKFKRAEDYPIDLYYLMDLSYSMKDDLENVKSLGTDLMNEMRRITSDFRI
GFGSFVEKTVMPYISTTPAKLRNPCTSEQNCTSPFSYKNVLSLTDKGEVENELVGKQRISGNLDSPEGGFDAIMQVAVCGSLIGWRNVTRLLV
FSTDAGFHFAGDGKLGGIVLPNDGHCHLENDVYTMSHYYDYPSIAHLVQKLSENNIQTIFAVTEEFQPVYKELKNLIPKSAVGTLSANSSNVI
QLIIDAYNSLSSEVILENSKLPEGVTINYKSYCKNGVNGTGENGRKCSNISIGDEVQFEISITANKCPNKNSETIKIKPLGFTEEVEIILQFI
CECECQSEGIPSSPKCHDGNGTFECGACRCNEGRVGRHCECSTDEVNSEDMDAYCRKENSSEICTNNGECVCGQCVCRKRDNTNEIYSGKFCE
CDNFNCDRSNGLICGGNGVCKCRVCECNPNYTGSACDCSLDTTSCMAVNGQICNGRGVCECGVCKCTDPKFQGPTCEMCQTCLGVCAEHKECV
QCRAFNKGEKKDTCAQECSHFNITKVENRDKLPQPGQVDPLSHCKEKDVDDCWFYFTYSVNGNNEATVHVVETPECPTGPDIIPIVAGVVAGI
VLIGLALLLIWKLLMIIHDRREFAKFEKEKMNAKWDTGENPIYKSAVTTVVNPKYEGK (SEQ ID NO: 157)
>tr|F1MX12|F1MX12_BOVIN Ankyrin repeat domain 2 OS = Bos taurus OX = 9913 GN = ANKRD2 PE = 4
SV = 2
WQKHLAGRGWGPWHIKPPGPAEAGCDGTMADSEEVQRATALIEERLAQEEENEKLRGTTHQKLPMEMLVLEDEKHHRPESPSLQK
VKGQERVRKTSLDLRREIIDVGGIQNLIQLRKKRKQKKREALAASQEPPPEPEEITGPVDEETFLKAAVEGKMKVIEKFLADGGSPDTCDQFR
RTALHRASLEGHMEILEKLLESGATVDFQDRLDCTAMHWACRGGHLEVVRLLQSRGADTNVRDKLLSTPLHVAVRTGQVEIVEHFLSLGLDIN
AKDREGDSALHDAVRLNRYKIIKLLLLHGADMMSKNLAGKTPTDLVQLWQADTRHALENPEPGSEQNGLEGSTESGRETPQPVAAE (SEQ
ID NO: 158)
>tr|F1NG08|F1NG08_CHICK Ankyrin 2 OS = Gallus gallus OX = 9031 GN = ANK2 PE = 4 SV = 3
MAAPAPPAPGGGTSPPAGPPRLRQSDSNASFLRAARAGNLDKVVEYLKSGIDINTCNQNGLNALHLAAKEGHVGLVQELLERGSA
VDSATKKGNTALHIASLAGQAEVVKVLVKEGANINAQSQNGFTPLYMAAQENHIEVVKYLLENGANQSTATEDGFTPLAVALQQGHNQAVAIL
LENDTKGKVRLPALHIAARKDDTKSAALLLQNDHNADVQSKMMVNRTTESGFTPLHIAAHYGNVNVATLLLNRGAAVDFTARNGITPLHVASK
RGNTNMVKLLLDRGGQIDAKTRDGLTPLHCAARSGHDQVVELLLERGAPLLARTKNGLSPLHMAAQGDHVECVKHLLQHKAPVDDVTLDYLTA
LHVAAHCGHYRVTKLLLDKRANPNARALNGFTPLHIACKKNRIKVMELLVKYGASIQAITESGLTPIHVAAFMGHLNIVLLLLQNGASPDVTN
IRGETALHMAARAGQVEVVRCLLRNGALVDARAREEQTPLHIASRLGKTEIVQLLLQHMAHPDAATTNGYTPLHISAREGQVDVASVLLEAGA
SHSMSTKKGFTPLHVAAKYGSLEVAKLLLQRRASPDSAGKNGLTPLHVAAHYDNQKVALLLLEKGASPHATAKNGYTPLHIAAKKNQMQIATT
LLNYGAETNILTKQGVTPLHLASQGGHTDMVTLLLEKGSNIHVATKTGLTSLHLAAQEDKVNVAEILTKHGANQDAQTKLGYTPLIVACHYGN
IKMVNFLLKQGANVNAKTKNGYTPLHQAAQQGHTHIINVLLQHGAKPNAITTNGNTALAIARRLGYISVVDTLKVVTEEITTTTTTVTEKHKL
NVPETMTEVLDVSDEEAFKHSDDERFSDGEVYNGTGVISRNSWSDDTMTGDGGEYLRPEDLKELGDDSLPSSQFLDGMNYLRYSLEGGRSDSL
RSFSSDRSHTLSHASYLRDSAMIDDTVVIPSQQVTTLAKEAERNSYRLSWGPENLDNVALSSSPIHSGCSSPCLDHDNSSFLVSFMVDARGGA
MRGCRHNGLRIIIPPRKCTAPTRVTCRLVKRHRLATMPPMVEGEGLASRLIEVGPSGAQFLGPVIVEIPHFAALRGKERELVILRSENGDSWK
EHFCEYTEDELNEILNGMDEVLDTPEELEKKRICRIITRDFPQYFAVVSRIKQDSNLIGPEGGVLSSTVVPQVQAVFPEGALTKRIRVGLQAQ
PMHTELIKKILGNKATFSPIVTLEPRRRKFHKPITMTIPVPKASSDGIMNGYGGDTPTLRLLCSITGGTTPAQWEDITGTTPLTFVNECVSFT
TNVSARFWLIDCRQTQESVTFASQVYREIICVPYMAKFVVFAKSHDPIEARLRCFCMTDDKVDKTLEQQENFAEVARSRDVEVLEGKPIYVDC
FGNLVPLTKSGQHHIFSFFAFKENRLPLFVKVRDTTQEPCGRLSFMKEPKSTRGLVHQAICNLNITLPVYTKESESDQEQEEEVDMTSEKNQQ
DDRERTEERLAHIADHLGFSWTELARELDFTEEQIHQIRIENPNSLQDQSHALLKYWLERDGKHATDTSLTQCLTKINRMDIVHLMETSGIDS
MQVHGTRTYTEIEQTIGLDHSEGFSVLQEELYSSRHKPDERHRISKDGDPTEHPPIVSEEDVSVSYSPFQDSTPRSEAELSMAELLRQTHKEQ
VEAEFSGKPQDVIETTSSQHEYFVTTPGTEQRAASDTSARFSATKEEREKTSPQSPSSAQRGGSPIIQEPEELHLHQDDPSPRRTSLVIVESI
DEQPEKLGSGYEEESLEKELAEELGELENSSDEDEMVTTRVVRRRVIIQADSMPEMPPETVTEEQYTDEHGHTVVKKVTRKIIRRYVSPDGTE
KEDIIMQGTPQKPVTVEEGDGYSKVVKRVVLKSDSEQSEVTLSEPGVLPSASNFQSEPVEGRKVSKVIKTTVVQGERMEKHLGDASLATDLPS
AKEDFEEALSYTGNQIKIQLPALVEKEIMKEDGSIIKRTTLSKASTQKRTVMKDRYGKHVHIEELDDTPEALPQDDLQHDLQQLLRHFCKEDW
KOEAK (SEQ ID NO: 159)
>tr|A0A287AUI5|A0A287AUI5_PIG Ankyrin 2 OS = Sus scrofa OX = 9823 GN = ANK2 PE = 1 SV = 1
EGQSQDKGSKSGSSIQSLFFFSQSDSNASFLRAARAGNLDKVVEYLKGGIDINTCNQNGLNALHLAAKEGHVGLVQELLGRGSSV
DSATKKGNTALHIASLAGQAEVVKVLVKEGANINAQSQNGFTPLYMAAQENHIDVVKYLLENGANQSTATEDGFTPLAVALQQGHNQAVAILL
ENDTKGKVRLPALHIAARKDDTKSAALLLQNDHNADVQSKMMVNRTTESGFTPLHIAAHYGNVNVATLLLNRGAAVDFTARNGITPLHVASKR
GNTNMVKLLLDRGGQIDAKTRDGLTPLHCAARSGHDQVVELLLERGAPLLARTKNGLSPLHMAAQGDHVECVKHLLQHKAPVDDVTLDYLTAL
HVAAHCGHYRVTKLLLDKRANPNARALNGFTPLHIACKKNRIKVMELLVKYGASIQAITESGLIPIHVAAFMGHLNIVLLLLQNGASPDVTNI
RGETALHMAARAGQVEVVRCLLRNGALVDARAREEQTPLHIASRLGKTEIVQLLLQHMAHPDAATTNGYTPLHISAREGQVDVASVLLEAGAA
HSLATKKGFTPLHVAAKYGSLDVAKLLLQRRAAADSAGKNGLTPLHVAAHYDNQKVALLLLEKGASPHATAKNGYTPLHIAAKKNQMQIASTL
LNYGAETNIVTKQGVTPLHLASQEGHTDMVTLLLDKGANIHMSTKSGLTSLHLAAQEDKVNVADILTKHGADQDAHTKLGYTPLIVACHYGNV
KMVNFLLKQGANVNAKTKNGYTPLHQAAQQGHTHIINVLLQHGAKPNATTANGNTALAIAKRLGYISVVDTLKVVTEEVTTTTTTITEKHKLN
VPETMTEVLDVSDEEGDDTMTGDGGEYLRPEDLKELGDDSLPSSQFLDGMNYLRYSLEGGRSDSLRSFSSDRSHTLSHASYLRDSAMIDDTVV
IPSHQVSALAKEAERNSYRLSWGTENLDNVALSSSPIHSGFLVSFMVDARGGAMRGCRHNGLRIIIPPRKCTAPTRVTCRLVKRHRLATMPPM
VEGEGLASRLIEVGPSGAQFLGKLHLPTAPPPLNEGESLVSRILQLGPPGTKFLGPVIVEIPHFAALRGKERELVVLRSENGDSWKEHYCEYT
EDELNEILNGMDEVLDSPEDLEKKRICRIITRDFPQYFAVVSRIKQDSNLIGPEGGVQAVFPEGALTKRIRVGLQAQPMHSELVKKILGNKAT
FSPIVTLEPRRRKFHKPITMTIPVPKASSDVMLNGFGGDAPTLRLLCSITGGTTPAQWEDITGTTPLTFVNECVSFTTNVSARFWLIDCRQIQ
ESVTFASQVYREIICVPYMAKFVVFAKSHDPIEARLRCFCMTDDKVDKTLEQQENFAEVARSRDVEVLEGKPIYVDCFGNLVPLTKSGQHHIF
SFFAFKENRLPLFVKVRDSTQEPCGRLSFMKEPKSTRGLVHQAICNLNITLPIYTKESESDQEQEEEVIVRHYDETESTETSVLKSHLVNEVP
VLASPDLLSEVSEMKQDLIKMTAILTTDVSDRAGSLKVKELVKAAEEEPGEPFEIVERVKEDLEKVNEILRSGTCAGDEGSEPRSQPEREVVE
EEWVIVSDEEIEEAKRKAPLEITEYPCVEVRLDKDTKVKVEKDSLGLVNYLTEDLNSYVPPHGEPLQMEREKQEALGPGRSSESEGKDAPSEE
TQSTQKQPKPSLGIKKPVRRKLKDKQKQKEDSSQASADKSELKKGSSEESLDEDTGLAPEPLPAVKATSPLIEETPIGSIKDKVKALQKRVED
EQKGRSKLPVRVKGKEDVPKKITHRTQLAASPSLKSERHALASKPERHSSLSSPAKTERHPPVSPSSKTEKHSPVSPSAKTERHSPVSSSSKT
EKHSPVSPSTKPDRHSPVSSATKTERHPPVSPSGKTDKRPPVSPSGRTEKHPPVSPGRTEKRLPVSPAGRMERHSPMSTSGKTEKHLPVSPSG
KTDKQPPVSPTSKTERIEETMSVRELMKAFQSGQDPSKHKTGLFEHKSAKQKQPQEKGKVRGEKEKGLTVTQKETQKTETQTIKRSQRFLVTG
PQNPEEQPVVKREEGAGERGKALNHKTPEPVQSAPEEESHKVESPKEKLADEQGDMDLQISPDRKTSTDFSDVIKQELEDNDKYQEFCHNQVL
TSPFNTTFPLDYMKEEFLPALSLQSGALDGSSESLKHEGAAGSPCGSLMEGTPQISSEESYKHEGLAETPETSPESLSFSPKKTEEQTEETKE
TTKVESPPEIHSEKEDPSTKDVTDSSAGQGAVVTGGTEPSAECLLKEATLEPSKDTCPQPEDEGLDSQGESLAKETPKGLTEGVPHGEDPLTH
VSSAHEKQTDTEAQKSTASKPSDETAASPLPDAVVKTSPGTESKPQGVIRSPQGLELALPSRDSEVFSPGADESFAVSHKDSLEASPVLEDNS
SHKTPDSLEPSPLKESPCRDSLESSPVEPKMKAGILPSQFPLPSAVAKTELITEVASMRSRLLRDPDGSAEDDSLEQTSLMESSGKSPLSPDT
PSSEEISYEVTPKTTDAGTPKPAVIPECAEEDDLENGEKKRFTPEEEMFKMVTKIKISKQKRDYKKEPKQEDSSSSSDADVECSADLDEPKCM
ESVEDKSNAPVVVTAESRKASSSSESEPELTQLKKGADSGLLPEPVIRVQPPSPLPSSIDSNSSPEEVQFQPIVSKQYTFKMNEDTQEDSGKS
EEEEGCESHLPEDSHTVSTSGPEMSYDDVNRDADQPKICGSYGCEAESPSSSAIPVTLGLHSSTGEDVHEQLVIHKEPLALQDTGEKDTEGEE
LDVSRVEAPQVGYPTESSSSSSSLPHCPASEGKELDEDVVSTSSATKMEVTKADQAFESLPDDYSTQDLPNTTQTDSSSLDVPVSDPTETDDI
SDPQVTSPYENVPSQSFFSSEESKTQTDTRHTSFHSSEVCSVTTTSSVEEVVVTSSSGRTVSSQESNLEDQNLECKQESPLCEVQPDGAPSSL
EPAAPTTSTVVGEQISKVIITKTDVDSDSWSEIREDDEAFEARVKEEEQKIFGLMVDRQSQGTTPDTTPARTPTEEGTPTSEQNPFLFQEGKL
FEMTRSGAIDMTKRSYGDESFHFFQIGQESREETLTEDMKEGGTGTELPQLETSAESLAPLESKETVNDEADLVPDDLSEEVEEVPTSDGQLN
SQMGISASTETSMKEAASAGAEDLPCIQMSDTPSLSLVKQAALQLDFSTVTRSVYSDRDDESPDSSPEEQKSVIEIPTAPMENVPSTESRSKI
PVRTMPTSTPAFPSMECESAPSEGFLPSLDEESQEDETKPKSKIPVKAPVPRVEQQLLHLDSSLQETVAPQGQDVTSRAPDSRSKSESDASPL
DAKITCPEKTGSYTETDLESSEGTEELELESEDETTRPKIFASRLPVKSKSTTSSCRGATSPTKESKEHFFDLYRNSIEFFEEISDEASKLVD
RLTQSEREQELVSDDESSSALEVSVIENLPPVETEQSIPEDIFDTRPIWDESIETLIERIPDENGHDHAEDQQDEQERIEERLAYIADHLGES
WTELARELDFTEEQIHQIRIENPNSLQDQSHALLKYWLERDGKHATDINLIECLTKINRMDIVHLMETITEPLQERISHSYAEIEQTITLDHS
EGFSVLQEELCTAQNKQKEEQAASKEGESYDHPPIVSEEDISVGYSTFQDSIPKTEGDGSVTELLPKTHEEQVQQDFSGKMQDLPEESSLEQQ
EYFVTTPGTEASETPKATAAPGSPSKTPEEIITPPEEERPYLQTPTASEQGDSPIVQEPEEPPEHREASLPQKTSLVIVESADDQPQTFERLD
EDAAFQKGDDMPDIPPETVTEEEYVDEHGHTVVKKVTRKIIRRYVSSDGTEKEEMTMQGAPQDPISIEEGDGYSKVIKRVVLKSDTERSEVRA
DEVERCK (SEQ ID NO: 160)
>sp|P49024|PAXI_CHICK Paxillin OS = Gallus gallus OX = 9031 GN = PXN PE = 1 SV = 1
MDDLDALLADLESTTSHISKRPVFLTEETPYSYPTGNHTYQEIAVPPPVPPPPSSEALNGTVIDPLDQWQPSVSRYGHQQPQSQS
PIYSSSAKSSSASVPRDGLSSPSPRASEEEHVYSFPNKQKSAEPSPTMTSTSLGSNLSELDRLLLELNAVQHNPPSGFSADEVSRSPSLPNVT
GPHYVIPESSSSAGGKAAPPTKEKPKRNGGRGIEDVRPSVESLLDELESSVPSPVPAITVSQGEVSSPQRVNASQQQTRISASSATRELDELM
ASLSDFKFMAQGKAGGSSSPPSTTPKPGSQLDTMLGSLQSDLNKLGVATVAKGVCGACKKPIAGQVVTAMGKTWHPEHFVCTHCQEEIGSRNF
FERDGQPYCEKDYHNLFSPRCYYCNGPILDKVVTALDRTWHPEHFFCAQCGVFFGPEGFHEKDGKAYCRKDYFDMFAPKCGGCARAILENYIS
ALNTLWHPECFVCRECFTPFINGSFFEHDGQPYCEVHYHERRGSLCSGCQKPITGRCITAMGKKFHPEHFVCAFCLKQLNKGTFKEQNDKPYC
QNCFLKLFC (SEQ ID NO: 161)
>tr|F1MFD1|F1MFD1_BOVIN Paxillin OS = Bos taurus OX = 9913 GN = PXN PE = 1 SV = 2
ALLADLESTTSHISKRPVFLSEETPYSYPTGNHTYQEIAVPPVPPPPSSEALNGSVLDPLDPWPPSTSRFTHQQPQSSSPVYGSS
AKTSSASNPQDGGGPPCPRAGEEDHVYSFPNKQKSAEPSPTVMSSSLGSNLSELDRLLLELNAVQHNPPGFPADEANSSPPLPGPLSTHYGVP
ENNSLLGGKAGALTKEKPKRNGGRGLEDLRPSVENLLDELESSVPSPVPTITVNQGEMSSPQRVTSSQQQTRISASSATRELDELMASLSDLS
KIQDLEQRADGELCWAAGWPLNGRQSGPEGQDMGGFMAQGKTGSSSPPGGPPKPGSQLDSMLGSLQSDLNKLGVATVAKGVCGACKKPIAGQV
VTAMGKTWHPEHFVCTHCQEEIGSRNFFERDGQPYCEKDYHNLFSPRCYYCNGPILDKVVTALDRTWHPEHFFCAQCGAFFGPEGFHEKDGKA
YCRKDYFDMFAPKCGGCARAILENYISALNTLWHPECFVCRECFTPFVHGSFFEHEGQPYCEAHYHERRGSLCSGCQKPITGRCITAMAKKFH
PEHFVCAFCLKQLNKGTFKEQNDKPYCQNCFLKLFC (SEQ ID NO: 162)
>tr|A0A287BTJ4|A0A287BTJ4_PIG Paxillin OS = Sus scrofa OX = 9823 GN = PXN PE = 4 SV = 1
DALLADLESTTSHISKRPVFLSEETPYSYPTGNHTYQEIAVPPPVPPPPSSEALNGSVLDPIDQWQPSTSRFIHQQPQAPSPVYG
SSAKTSSSSNPQDGIGLPCPRASEEEHVYSFPNKQKSAEPSPTVMSSSLGSNLSELDRLLLELNAVQHNAPGFPTDEANSSPPLPGALSPHYG
VLEHNSSLGGKAGPVTKEKPKRNGGRGLEDVRPSVESLLDELESSVPSPVPAITLNQGEMNSPQRVTSSQQQTRISASSATRELDELMASLSD
FKFMAQGKTGSSSPPGGPPKPGSQLDSMLGSLQSDLNKLGVATVAKGVCGACKKPIAGQVVTAMGKTWHPEHFVCTHCQEEIGSRNFFERDGQ
PYCEKDYHNLFSPRCYYCNGPILDKVVTALDRTWHPEHFFCAQCGAFFGPEGFHEKDGKAYCRKDYFDMFAPKCGGCARAILENYISALNTLW
HPECFVCRECFTPFVNGSFFEHDGQPYCEVHYHERRGSLCSGCQKPITGRCITAMAKKFHPEHFVCAFCLKQLNKGTFKEQNDKPYCQNCELK
LFC (SEQ ID NO: 163)
>tr|O57613|O57613_CHICK Paranemin OS = Gallus gallus OX = 9031 PE = 2 SV = 2
MLSMEGFVGARALGEESLQMWDLNKRLEAYLARVKFLEEENEGLRAEIQSTKENPAGDPRRARYEEELRSLRDALHRAFTEKCAA
ELARDNLYEEVQHVRSRCQKEQAAREEAKRQLSSSKKELEEERRAQIWLKERAVQLEKEVEALLEVHEEEKAGLDQELASFSQSLEGFRCAPV
AFQPVEVEDYSKRLSEIWRGAVETYKAEVSQLERALGQAKENLWQVAEDNQQSQLQLRHLEKELVGLKVRKEMLEESLGQQWQEQHGEAEKFQ
LAIEALEQEKQSLQVQIAQVLEDRQQLMHLKMSLSLEVATYRTLLEAESTRLQMPPGEFKLANSLRDVKLEASSSKHRASLAAFPRPEGVAQL
CRTPGDALKVLTPKSKSSSALEFQKISSVLQAPRSWEPAAPSPTVPVVSPEPGSGGAESPVHECGAGKESPMLSPLSPEQLVNHALQDALKEM
QDDAEAKEVPTLSATQSTRDGDLEATMEEEEAAGTQGVGAEGETVSPPGLCFCSNEPTLLSATQSDVESQEEMWEEERSKEEMLNPLSSMESQ
EPGGEPWGGVTRRSRLQVGKEDMEATSTEALHISEKKEQREIWSPSREDEECEFPDEEREMQEEGSLQMEIEAACAVPVGSHPVLPTGIHLQE
DFLEREQESEHQETSLGELGAAAGEEREQEVCQELKASSIEEAMPAAEGSSGSGEGTTGRESTGRARDDEGEEEDKGREALGEDDLQAGEALG
AKELGKESMGLEEAEGMWEESVDLQEEHRDLQEGHGDLQVEHEDLWEEHGHIQEEHGDTQEEYGDTQEEHGDLQVEGGDLQEEHGDTQEEHGD
LQEEHGDTQEEHGDLQVEHEDLQVEHGDLWEEHRDVQEEHGDTQEEYGDTQEEHGDLQVEGGDLQEEHGDTQEEHEDLQEEHGDTQEEHGDLQ
VEHGDLQEEHRDLQEGHGDLKEEHGDLQVEHEDLQVEHGDLQEEHGDTQEEHGDLQEEHGDLQVEHEDLQVEHGDLQVEHGDLQEEHGDTQEE
HRDLQEVHGDQQEEHRDLQEGHGDLQEEHGDLQVEHGDLQEEYGDTQEEHRDLQEVHGDQQEEHRNLQEGHGDLQEGHGDLQEEHGDLWKEHG
VLKEEHGDLQEGHGDLQEESGDPQEEPGEPWVQHGEQGSAGDGLEQDMVLQPGEGAWGREDNDISQKQQAQDWEGTAEDEEETGVNTITSQEP
TQVDDNPHAEAAENEERDVTSPTAMEETQEGEDEGDAGSEVQSQQQPQDTPGQEAEPAPGQKEVRYGDTGEAPGDPQEPAEALEVEDEELSSE
PIELERGSPDTAVMQQDLGNGAESDEPMEEDFQSEDAQLEEPKACRMELEDTLLNSTPLCAYSGEMLESDPNPPSSGGDGEAAPEMAQEEEGD
LRGSDEAAVHAEPESCEELSPAPECTEEEEGYFIVSAPSQEGSSMEEAENSEEFEEIKVEAAEDRKDELTAPGVASLVPEDEGHSEPFVGEAE
DVKMPLGEFEMPKEEDEEDAGGFAAELEEGLTVPVAEGLSEGHTDKTTLGDEGLGEEDVQDGDNPPATETSDTDPSNTDPPPGTMLEHGAGME
AAEYLPDVPTQLPVDIMKDSDILEIVEQALEFNQELVLGAKLAKDGQEEDGDAQPPQEEEGGSSPTSSCDEQPTVQEAVAEPERTKNGEQNGL
HRQASLEDLAEFTEEGLNGITHPGEAPAAHTLPLPSKHSGAEPVPELSPLQTTSCARSRSPGPLGRSDAVGPHSPATIGRLP (SEQ ID
NO: 164)
>tr|E7EYD0|E7EYD0_DANRE Myomesin 1a (skelemin) OS = Danio rerio OX = 7955 GN = myomla PE = 4
SV = 1
MSESLKLQKEQEERDLETRYESSYHQSSLSSVSRQSYTAYVSSHGHKISGKKKEKKLTPLSKKEKRSYLAVDKEDEVIGYVVPVF
RSSHLATQDLMETQEEVKEEGVGYVVMRNLFARESGFKMRTVKKTRETSMRESAERMALSQKLHEKEQFQKKMNPDSLTHPPEFIVKPRGQTV
WEGKSVTLHCTVAGWPKPRVAWYKNNVLIDAKARPEKYFTESQYNMHSLEIKNCTFSDTAEYRISALNVKGESSAFAPVIIKRFKEEEELVER
PHGYSPEYGVTFNTTIIDKFDVSFGREGETMSLGCTVIVYPTVKRYQPEIVWYRNGVALSSSKWVHMHWSGEKATLTLVHLNKEDEGLYTLRV
NTKSGFDTHSAYVFVRDADVEEEGVPVAPLDVRCHDANKDYVVVTWKQPAVEGSSAITGYFIDRLEVGNHHWTQCNDTPVKYARFPVTGLIEG
RSYMFRVRALNNAGVSRPSRVSDPVVAMDPSDRARLRAGPSAPWTGVIKFTEEDPTVGVIPGPPTDLAVTEATKSYVVLSWKPPVQRGHEGVM
YYVEKCLVGTDAWQRVNTGIPVKSPRFALFDLAEDKSYTFRVRSCNSAGVGEPSEETGATTVGDRLDLPSAPGPVIPIRNTDSSVVVCWGASK
EVKDLVGYYIEVTVDGSGVWAPCNNKPVKGTRFVCHGLNATDKCTFRVKAVNAAGYSGSSAESEACLVKASIAVPSPPTGVTTLERLRDYMVI
GWQAPAKTGGADIRGYYLDYRTVKDGVTSKWHELNLKAVTSSPYKVTDLKENEFYQFQVRAFNQAGISEASIPTAPLECKEWTVAVPGPPHGL
RVQEVRKDSMVVLWEPPTFNGRSPVTGYYVDFKEENGRWRCVQERSTKHTYMKVSGLQEGISYRLRIHAKNLAGVGVPSKATDAILAETRPGT
NEIVVDVDDNGVISLIFECSDLKEDSQFVWSKNYEAFTDSSRLTIQTTGGKSRAIFNDPSLDDLGIYSCVVTNTDGVSASYTLTEEGLKRLLD
ISHDHQFPIIPFKSEMAIELQEKGRVRFWAEVGKFTSNLQVEYVENDNVIHEGKKYTMNFNKSTGIIEMFMDLLEVTDEGTFTFNLVDGKATG
RTSLVLIGEEFAELQKKSEFERAEWVRRQGPHFVEYLSFEVTPECDVHLKCKVGNIKPATEIAWFKDGIEIEEDDEDAKKIGKSDEVLTFDIG
KLVIKSEKAERKKKPATEESPSKPKISKKDAGVYEVKLKDERGKDKTLLNLTDAGYQAVLNEVERVIANSSTELKVMSTEHGIILYSFVVHYL
EDLRVGWLHKESKISHSDRVQCGVTGEQLWLKINEPTEKDKGKYAIDIFDGKGSVKRVLDLSGQVWEEAFEEFKRLKAAAIAERNRARVVGGL
PDVVTIQEGKSLNLTGNVWGDPAPEVSWIKNEKPLVCDEHHTLKYEHSKFASITIAAVTTTDSGKYALLVKNKYGTEAAEFTVSVYIPEDEAE
KKE (SEQ ID NO: 165)

1-20. (canceled)

21. A composition comprising:
a host Saccharomyces cerevisiae cell transformed with an expression vector comprising genes encoding at least two different recombinant vertebrate animal muscle proteins, wherein expression of the at least two different recombinant vertebrate animal muscle proteins is driven by fungi promoters,
wherein:
(a) multiple copies of the expression vector are integrated into the genome of the host Saccharomyces cerevisiae cell;
(b) the at least two different recombinant vertebrate animal muscle proteins are not isolated from the host Saccharomyces cerevisiae cell;
(c) the composition is substantially free of antibiotics, animal growth hormones, and processed animal meat; and
(d) the composition is formulated for a cat or a dog in a dry kibble form.

22. The composition of claim 21, wherein the fungi promoters are selected from the group consisting of: promoters of the genes phosphoglycerate kinase (PGK, PGK1, PGK3), enolase (ENO, ENOI), glyceraldehyde-3-phosphate dehydrogenase (gpdA, GAP, GAPDH), hexokinase, pyruvate decarboxylase, phosphofructokinase, glucose-6-phosphate isomerase, 3-phosphoglycerate mutase, pyruvate kinase (pki1), triosephosphate isomerase, phosphoglucose isomerase, glucokinase, alcohol dehydrogenase promoter (ADH1, ADH2, ADH4), isocytochrome C, acidic phosphatase, galactose metabolism enzymes GAL (GALI, GAL2, GAL3, GAL4, GAL5, GAL6, GAL7, GAL8, GAL9, GAL10), alternative oxidase (AOD), alcohol oxidase I (AOX1), alcohol oxidase 2 (AOX2), CUP1, AHSB4m, adhl+, AINV, alcA, AXDH, cellobiohydrolase I (cbhl), ccg-1, cDNA1, cellular filament polypeptide (cfp), cpc-2, ctr4+, dihydroxyacetone synthase (DAS), FMD, formate dehydrogenase (FMDH), formaldehyde dehydrogenase (FLD1), GAA, GCW14, glucoamylase (glaA, gla-1), inv1, isocitrate lyase (ICL1), glycerol kinase (GUT1), acetohydroxy acid isomeroreductase (ILV5), β-galactosidase (lac4), LEU2, melO, MET3, MET25, KAR2, KEX2, methanol oxidase (MOX), nmtl, peroxin 8 (PEX8), pcbC, PET9, PH05, PH089, PYK1, phosphatidylinositol synthase (PIS1), RPS7, TEF, translation elongation factor 1 alpha (TEF1), sorbitol dehydrogenase (SDH), SSA4, THI11, homoserine kinase, XRP2, TPI, and YPT1, PHO5, CYC1, HISS, ADC1, TAP1, URA3, LEU2, TP1, TDH1, TDH3, FBA1, ADR1, TPI1, and any combination therein.

23. The composition of claim 21, wherein the origin of each of the at least two different recombinant vertebrate animal muscle proteins is selected from the group consisting of lion, bear, weasel, seal, wolf, coyote, fox, horse, donkey, pig, camel, cattle, deer, elephant, golden mole, manatee, primate, rabbit, hare, pika, rodent, mole, whale, bat, seal, hoofed mammal, platypus, echidna, koala, possum, tapir, kangaroo, wallaby, marsupial lion, antelope, reindeer, elk, sheep, goat, camel, carabao, yak, bison, buffalo, caribou, water buffalo, pig, horse, donkey, chicken, turkey, duck, pigeon, penguin, ostrich, goose, pheasant, quail, alligator, crocodile, shark, tuna, trout, salmon, jacks, carp, catfish, cod, flounder, bass, tilapia, sturgeon, frog, salamander, and toad.

24. The composition of claim 21, wherein each of the at least two different recombinant vertebrate animal muscle proteins comprise an amino acid sequence at least about 80% identical to a sequence in Table 1.

25. The composition of claim 21, wherein each of the at least two different recombinant vertebrate animal muscle proteins is a truncated form of a sequence in Table 1.

26. The composition of claim 21, wherein the composition consists of 5% to 40% recombinant animal protein, on a mass percentage basis.

27. The composition of claim 21, wherein the composition consists of 1% to 30% recombinant animal protein.
